SEQUENCE LISTING (60 sequences)

SEQ ID NO: 1 - protein, 211 aa, Homo sapiens [sequence omitted]

SEQ ID NO: 2 - DNA, 1308 bp, Homo sapiens
tccgcaaatatgcagaattaccggccgggtcgctcctgaagccagcgcggggaggcagcgcggcggcggccagcaccgggaacgcaccgaggaagaagcccagcccccgccctccgccccttccgtccccacccccatcccggcggcccaggaggctccccgcgctggcgcgcactccctgtttctcctcctcctggctggcgctgcctgcctctccgcactcactgctcgccgggcgccgtccgccagctccgtgctccccgcgccaccctcctccgggccgcgctccctaagggatggtactgattttcgccgccacaggagaccggctggagcgccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaaagcagccaaccagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagaaaagaaaaaaaaaggaattc

SEQ ID NO: 3 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 4 - DNA, 2137 bp, Homo sapiens
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 5 - protein, 155 aa, Homo sapiens [sequence omitted]

SEQ ID NO: 6 - DNA, 3877 bp, Homo sapiens
gccagattagcggacgcgtgcccgcggttgcaacgggatcccgggcgctgcagcttgggaggcggctctccccaggcggcgtccgcggagacaaccatccgtgaaccccaggtcccggcgcgccggctcgccgcgcaccaggggccggcggacagaagagcggccgagcggctcgaggctgggggacccggcgcggccgcgcgctgccgggcgggaggctggggggccggggcggggccgtgccccggagcgggtcggaggccggggccggggccgggggacggcggctccccgcgcggctccagcggctcggggatcccggccgggccccgcaggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtaaagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaccttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctgtttttcccaaaaggtaaaaatatagattgaaaagttaaaacattttgcatggctgcagttcctttgtttcttgagataagattccaaagaacttagatttatttcttcaacaccgaaatgctggaggtgtttgatcagttttcaagaaacttggaatataaataattttataattcaacaaaggttttcacattttataaggttgatttttcaattaaatgcaaatttatgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctgaaagcttcacaattgtcacagacaaagatttttgttccaatactcgttttgcctctattttacttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcgtctgccatttttcaattacatgctgacttcccttacaattgagatttgcccataggttaaacatggttagaaacaactgaaagcataaaagaaaaatctaggccgggtgcagtggctcatgcccatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttacagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagacataggaaaaactaagaggttttatttttgtttttgctgatgaagagatatgtttaaatactgttgtattgttttgtttagttacaggacaataatgaaatggagtttatatttgttatttctattttgttatatttaataatagaattagattgaaataaaatataatgggaaat

SEQ ID NO: 7 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 8 - DNA, 2312 bp, Homo sapiens
tccagtgacggagccgcccggccgacagccccgagacgacagcccggcgcgtcccggtccccacctccgaccaccgccagcgctccaggccccgcgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcttccccagacactggtttgaagaatgttaagacttgacagtggaactacattagtacacagcaccagaatgtatattaaggtgtggctttaggagcagtgggagggtaccagcagaaaggttagtatcatcagatagctcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctctcagatagaatgacagtccgtcaaaacagattgtttgcaaaggggaggcatcagtgtccttggcaggctgatttctaggtaggaaatgtggtagctcacgctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtatattttttctaaaaaaaaaaaaaaacgacagcaacggaattc

SEQ ID NO: 9 - protein, 250 aa, Homo sapiens
MRGTPKTHLLAFSLLCLLSKVRTQLCPTPCTCPWPPPRCPLGVPLVLDGCGCCRVCARRLGEPCDQLHVCDASQGLVCQPGAGPGGRGALCLLAEDDSSCEVNGRLYREGETFQPHCSIRCRCEDGGFTCVPLCSEDVRLPSWDCPHPRRVEVLGKCCPEWVCGQGGGLGTQPLPAQGPQFSGLVSSLPPGVPCPEWSTAWGPCSTTCGLGMATRVSNQNRFCRLETQRRLCLSRPCPPSRGRSPQNSAF

SEQ ID NO: 10 - DNA, 1309 bp, Homo sapiens
  misc_feature (1061): n is a or g or c or t/u
ggggacatgagaggcacaccgaagacccacctcctggccttctccctcctctgcctcctctcaaaggtgcgtacccagctgtgcccgacaccatgtacctgcccctggccacctccccgatgcccgctgggagtacccctggtgctggatggctgtggctgctgccgggtatgtgcacggcggctgggggagccctgcgaccaactccacgtctgcgacgccagccagggcctggtctgccagcccggggcaggacccggtggccggggggccctgtgcctcttggcagaggacgacagcagctgtgaggtgaacggccgcctgtatcgggaaggggagaccttccagccccactgcagcatccgctgccgctgcgaggacggcggcttcacctgcgtgccgctgtgcagcgaggatgtgcggctgcccagctgggactgcccccaccccaggagggtcgaggtcctgggcaagtgctgccctgagtgggtgtgcggccaaggagggggactggggacccagccccttccagcccaaggaccccagttttctggccttgtctcttccctgccccctggtgtcccctgcccagaatggagcacggcctggggaccctgctcgaccacctgtgggctgggcatggccacccgggtgtccaaccagaaccgcttctgccgactggagacccagcgccgcctgtgcctgtccaggccctgcccaccctccaggggtcgcagtccacaaaacagtgccttctagagccgggctgggaatggggacacggtgtccaccatccccagctggtggccctgtgcctgggccctgggctgatggaagatggtccgtgcccaggcccttggctgcaggcaacactttagcttgggtccaccatgcagaacaccaatattaacacgctgcctggtctgtctggatcccgagtatggcagaggtgcaagacctagtcctctttcctctaactcactgcctaggaggctggccaaggtgtccagggtcctctagcccactccctgcctacacacacagcctatatcaaacatgcnccccggcgagctttctctccgacttcccctgggcaagagatgggacaagcagtcccttaatattgaggctgcagcaggtgctgggctggactggccatttttctgggggtaggatgaagagaaggcacacagagattctggatctcctgctgccttttctggagtttgtaaaattgttcctgaatacaagcctatgcgtgaaaaaaaaaaaaaaaaaaaaaaaaaaaaaaaaaaaaaaaaaaaaaaaa

SEQ ID NO: 11 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 12 - DNA, 627 bp, Homo sapiens
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 13 - protein, 390 aa, Homo sapiens
MPPSGLRLLPLLLPLLWLLVLTPGPPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEELSNMIVRSCKCS

SEQ ID NO: 14 - DNA, 2745 bp, Homo sapiens
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggccaggcggggtgctcacgcctgtaatcctagcacttttgggaggcagagatgggaggatacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 15 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 16 - DNA, 789 bp, Homo sapiens
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggcacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatagcttctaaaaaaaaaaa

SEQ ID NO: 17 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 18 - DNA, 990 bp, Homo sapiens
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 19 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 20 - DNA, 4877 bp, Homo sapiens
actgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgccaaaatgagttgtgatgaatcaatgaaaaatgtaatttagaaactgatttcttcagaattagatggccttattttttaaaatatttgaatgaaaacattttatttttaaaatattacacaggaggccttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacat

SEQ ID NO: 21 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 22 - DNA, 1964 bp, Homo sapiens
aagcttcgggccagggtccacctgtccccgcagcgccgtcgcgccctcctgccgcaggccaccgaggccgccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaatgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagaagcttttggggagcagagacactaacgacttcagggcagggctctgatattccatgaatgtatcaggaaatatatatgtgtgtgtatgtttgcacacttgttgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctga

SEQ ID NO: 23 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 24 - DNA, 1970 bp, Homo sapiens
atattggagtagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcatgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgtttt

SEQ ID NO: 25 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 26 - DNA, 2733 bp, Homo sapiens
cctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcatcggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgatactggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgacgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacactgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccttggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggc

SEQ ID NO: 27 - protein, 477 aa, Homo sapiens [sequence omitted]

SEQ ID NO: 28 - DNA, 1434 bp, Homo sapiens
atgaagagtcttccaatcctactgttgctgtgcgtggcagtttgctcagcctatccattggatggagctgcaaggggtgaggacaccagcatgaaccttgttcagaaatatctagaaaactactacgacctcgaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgacgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggccatcagaggaaatgaggtacgagctggatacccaagaggcatccacaccctaggtttccctccaaccgtgaggaaaatcgatgcagccatttcggataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttga

SEQ ID NO: 29 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 30 - DNA, 1078 bp, Homo sapiens
aagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttattggttaaactcctttatggtgtgactgtgtcttattccatctatgagctttgtcagtgcgcgtagatgtcaataaatgttacatacacaaata

SEQ ID NO: 31 - protein, 467 aa, Homo sapiens [sequence omitted]

SEQ ID NO: 32 - DNA, 2223 bp, Homo sapiens
gctcgccagggaagggccctacccagaggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcatgcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagagcatagccacctaggggaggaggccgtgtgaagacagaggcagagattggagtgacgcatctccaagccaa

SEQ ID NO: 33 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 34 - DNA, 2334 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 35 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 36 - DNA, 1743 bp, Homo sapiens
aaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctctttcgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttcctt

SEQ ID NO: 37 - protein, 488 aa, Homo sapiens
MAPAAWLRSAAARALLPPMLLLLLQPPPLLARALPPDVHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 38 - DNA, 2247 bp, Homo sapiens
ccggggcggatggctccggccgcctggctccgcagcgcggccgcgcgcgccctcctgcccccgatgctgctgctgctgctccagccgccgccgctgctggcccgggctctgccgccggacgtccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctgcagggggatggggtggggtacaaccaccatgacaactgccgggagggccacgcaggtcgtggtcacctgccagcgactgtctcagactgggcagggaggctttggcatgacttaagaggaagggcagtcttgggacccgctatgcaggtcctggcaaacctggctgccctgtctcatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttcttttttttttttaaactgaggattgtcattaaacacagttgttttct

SEQ ID NO: 39 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDLHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 40 - DNA, 1778 bp, Homo sapiens
tagaagtttacaatgaagtttcttctaatactgctcctgcaggccactgcttctggagctcttcccctgaacagctctacaagcctggaaaaaaataatgtgctatttggtgagagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatctccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggctgtaatgttccccacctacaaatatgtcgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaaattaagtatatatattttggctcaaataaaattg

SEQ ID NO: 41 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 42 - DNA, 2698 bp, Homo sapiens
caagatgcatccaggggtcctggctgccttcctcttcttgagctggactcattgtcgggccctgccccttcccagtggtggtgatgaagatgatttgtctgaggaagacctccagtttgcagagcgctacctgagatcatactaccatcctacaaatctcgcgggaatcctgaaggagaatgcagcaagctccatgactgagaggctccgagaaatgcagtctttcttcggcttagaggtgactggcaaacttgacgataacaccttagatgtcatgaaaaagccaagatgcggggttcctgatgtgggtgaatacaatgttttccctcgaactcttaaatggtccaaaatgaatttaacctacagaattgtgaattacacccctgatatgactcattctgaagtcgaaaaggcattcaaaaaagccttcaaagtttggtccgatgtaactcctctgaattttaccagacttcacgatggcattgctgacatcatgatctcttttggaattaaggagcatggcgacttctacccatttgatgggccctctggcctgctggctcatgcttttcctcctgggccaaattatggaggagatgcccattttgatgatgatgaaacctggacaagtagttccaaaggctacaacttgtttcttgttgctgcgcatgagttcggccactccttaggtcttgaccactccaaggaccctggagcactcatgtttcctatctacacctacaccggcaaaagccactttatgcttcctgatgacgatgtacaagggatccagtctctctatggtccaggagatgaagaccccaaccctaaacatccaaaaacgccagacaaatgtgacccttccttatcccttgatgccattaccagtctccgaggagaaacaatgatctttaaagacagattcttctggcgcctgcatcctcagcaggttgatgcggagctgtttttaacgaaatcattttggccagaacttcccaaccgtattgatgctgcatatgagcacccttctcatgacctcatcttcatcttcagaggtagaaaattttgggctcttaatggttatgacattctggaaggttatcccaaaaaaatatctgaactgggtcttccaaaagaagttaagaagataagtgcagctgttcactttgaggatacaggcaagactctcctgttctcaggaaaccaggtctggagatatgatgatactaaccatattatggataaagactatccgagactaatagaagaagacttcccaggaattggtgataaagtagatgctgtctatgagaaaaatggttatatctattttttcaacggacccatacagtttgaatacagcatctggagtaaccgtattgttcgcgtcatgccagcaaattccattttgtggtgttaagtgtctttttaaaaattgttatttaaatcctgaagagcatttggggtaatacttccagaagtgcggggtaggggaagaagagctatcaggagaaagcttggttctgtgaacaagcttcagtaagttatctttgaatatgtagtatctatatgactatgcgtggctggaaccacattgaagaatgttagagtaatgaaatggaggatctctaaagagcatctgattcttgttgctgtacaaaagcaatggttgatgatacttcccacaccacaaatgggacacatggtctgtcaatgagagcataatttaaaaatatatttataaggaaattttacaagggcataaagtaaatacatgcatataatgaataaatcattcttactaaaaagtataaaatagtatgaaaatggaaatttgggagagccatacataaaagaaataaaccaaaggaaaatgtctgtaataatagactgtaacttccaaataaataattttcattttgcactgaggatattcagatgtatgtgcccttcttcacacagacactaacgaaatatcaaagtcattaaagacaggagacaaaagagcagtggtaagaatagtagatgtggcctttgaattctgtttaattttcacttttggcaatgactcaaagtctgctctcatataagacaaatattcctttgcatattataaaggataaagaaggatgatgtctttttattaaaatatttcaggttcttcagaagtcacacattacaaagttaaaattgttatcaaaatagtctaaggccatggcatccctttttcataaattatttgattatttaagactaaaagttgcattttaaccctattttacctagctaattatttaattgtccggtttgtcttggatatataggctattttctaaagacttgtatagcatgaaataaaatatatcttataaagtggaagtatgtatattaaaaaagagacatccaaatttttttttaaagcagtctactagattgtgatcccttgagatatggaaggatgcctttttttctctgcatttaaaaaaatcccccagcacttcccacagtgcctattgatacttggggagggtgcttggcacttattgaatatatgatcggccatcaagggaagaactattgtgctcagagacactgttgataaaaactcaggcaaagaaaatgaaatgcatatttgcaaagtgtattaggaagtgtttatgttgtttataataaaaatatattttcaacagaaaaaaaa

SEQ ID NO: 43 - protein, 582 aa, Homo sapiens [sequence omitted]

SEQ ID NO: 44 - DNA, 3403 bp, Homo sapiens
agttcagtgcctaccgaagacaaaggcgccccgagggagtggcggtgcgaccccagggcgtgggcccggccgcggagccacactgcccggctgacccggtggtctcggaccatgtctcccgccccaagaccctcccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaattactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccagggcccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgcttcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaagaagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccatccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctggggaactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatcttcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagccttaaggccacagagaaagaaccttgcccaaactcaggcagctggggctgaggcccaaaggcagaacagccagagggggcaggaggggaccaaaaaggaaaatgaggacgtgcagcagcattggaaggctggggcccggcagccaggttaaagctaacagggggccatcagggtgggcttgtggagctctcaggaagggccctgaggaaggcacacttgctcctgttggtccctgtccttgctgcccaggcagggtggaggggaagggtagggcagccagagaaaggagcagagaaggcacacaaacgaggaatgaggggcttcacgagaggccacagggcctggctggccacgctgtcccggcctgctcaccatctcagtgagggacaggagctggggctgcttaggctgggtccacgcttccctggtgccagcacccctcaagcctgtctcaccagtggcctgccctctcgctcccccacccagcccacccattgaagtctccttgggtcccaaaggtgggcatggtaccggggacttgggagagtgagacccagtggagggagcaagaggagagggatgtgggggggtggggcacgggtaggggaaatggggtgaacggtgctggcagttcggctagatttctgtcttgtttgtttttttgttttgtttaatgtatatttttattataattattatatat

SEQ ID NO: 45 - protein, 669 aa, Homo sapiens
MGSDPSAPGRPGWTGSLLGDREEAARPRLLPLLLVLLGCLGLGVAAEDAEVHAENWLRLYGYLPQPSRHMSTMRSAQILASALAEMQRFYGIPVTGVLDEETKEWMKRPRCGVPDQFGVRVKANLRRRRKRYALTGRKWNNHHLTFSIQNYTEKLGWYHSMEAVRRAFRVWEQATPLVFQEVPYEDIRLRRQKEADIMVLFASGFHGDSSPFDGTGGFLAHAYFPGPGLGGDTHFDADEPWTFSSTDLHGNNLFLVAVHELGHALGLEHSSNPNAIMAPFYQWKDVDNFKLPEDDLRGIQQLYGTPDGQPQPTQPLPTVTPRRPGRPDHRPPRPPQPPPPGGKPERPPKPGPPVQPRATERPDQYGPNICDGDFDTVAMLRGEMFVFKGRWFWRVRHNRVLDNYPMPIGHFWRGLPGDISAAYERQDGRFVFFKGDRYWLFREANLEPGYPQPLTSYGLGIPYDRIDTAIWWEPTGHTFFFQEDRYWRFNEETQRGDPGYPKPISVWQGIPASPKGAFLSNDAAYTYFYKGTKYWKFDNERLRMEPGYPKSILRDFMGCQEHVEPGPRWPDVARPPFNPHGGAEPGADSAEGDVGDGDGDFGAGVNKDGGSRVVVQMEEVARTVNVVMVLVPLLLLLCVLGLTYALVQMQRKGAPRVLLYCKRSLQEWV

SEQ ID NO: 46 - DNA, 3530 bp, Homo sapiens
gcgaggatccggcgtgcagtgttccgagctgggctgggcgccgagagcatgggcagcgacccgagcgcgcccggacggccgggctggacgggcagcctcctcggcgaccgggaggaggcggcgcggccgcgactgctgccgctgctcctggtgcttctgggctgcctgggccttggcgtagcggccgaagacgcggaggtccatgccgagaactggctgcggctttatggctacctgcctcagcccagccgccatatgtccaccatgcgttccgcccagatcttggcctcggcccttgcagagatgcagcgcttctacgggatcccagtcaccggtgtgctcgacgaagagaccaaggagtggatgaagcggccccgctgtggggtgccagaccagttcggggtacgagtgaaagccaacctgcggcggcgtcggaagcgctacgccctcaccgggaggaagtggaacaaccaccatctgacctttagcatccagaactacacggagaagttgggctggtaccactcgatggaggcggtgcgcagggccttccgcgtgtgggagcaggccacgcccctggtcttccaggaggtgccctatgaggacatccggctgcggcgacagaaggaggccgacatcatggtactctttgcctctggcttccacggcgacagctcgccgtttgatggcaccggtggctttctggcccacgcctatttccctggccccggcctaggcggggacacccattttgacgcagatgagccctggaccttctccagcactgacctgcatggaaacaacctcttcctggtggcagtgcatgagctgggccacgcgctggggctggagcactccagcaaccccaatgccatcatggcgccgttctaccagtggaaggacgttgacaacttcaagctgcccgaggacgatctccgtggcatccagcagctctacggtaccccagacggtcagccacagcctacccagcctctccccactgtgacgccacggcggccaggccggcctgaccaccggccgccccggcctccccagccaccacccccaggtgggaagccagagcggcccccaaagccgggccccccagtccagccccgagccacagagcggcccgaccagtatggccccaacatctgcgacggggactttgacacagtggccatgcttcgcggggagatgttcgtgttcaagggccgctggttctggcgagtccggcacaaccgcgtcctggacaactatcccatgcccatcgggcacttctggcgtggtctgcccggtgacatcagtgctgcctacgagcgccaagacggtcgttttgtctttttcaaaggtgaccgctactggctctttcgagaagcgaacctggagcccggctacccacagccgctgaccagctatggcctgggcatcccctatgaccgcattgacacggccatctggtgggagcccacaggccacaccttcttcttccaagaggacaggtactggcgcttcaacgaggagacacagcgtggagaccctgggtaccccaagcccatcagtgtctggcaggggatccctgcctcccctaaaggggccttcctgagcaatgacgcagcctacacctacttctacaagggcaccaaatactggaaattcgacaatgagcgcctgcggatggagcccggctaccccaagtccatcctgcgggacttcatgggctgccaggagcacgtggagccaggcccccgatggcccgacgtggcccggccgcccttcaacccccacgggggtgcagagcccggggcggacagcgcagagggcgacgtgggggatggggatggggactttggggccggggtcaacaaggacgggggcagccgcgtggtggtgcagatggaggaggtggcacggacggtgaacgtggtgatggtgctggtgccactgctgctgctgctctgcgtcctgggcctcacctacgcgctggtgcagatgcagcgcaagggtgcgccacgtgtcctgctttactgcaagcgctcgctgcaggagtgggtctgaccacccagcgctcctgctaacggtgctcagggggcgcctgtggttctgagatggctcccaggggctccctccgcccccaggtaggggcccctctcagccctcacacaccctgtctgccccgccctcattatttatgtccaggtgtttgttttgttttgtttttggcaccttacttgaccatttgtttctgtttccccgactggggcagggtgtttagaattttctaaatgtagttctgctccagacagggaattaggcccccatcatcctctggcttggccacagccaggggagcagaggggcagaggcccacattggaagagcagcacctcctcagcctgaaccccagggctgtaactgccaggctctctttgcccagttggagactgtctggcccccctggtcccctccttcccaagtgagtctctctgggccttaggaagagccttccacccaggggcagccccaggccaaaggggacctggaagggaggtgggccgtggcccttgagtccccattgaggcttggttccttcccaatccagtggacttcgcagtccacttctgacagcctcagtgaccctggctccttgtgccagagaacccagcccacccccggcagcagcccccagctcccacctccccttgggcccacaccttcttccctctctggagaaagggccctgggcctgcctcaccacggaccaaagggagtctgccagggcccctctccccagggaagcagcagcctcgcccctggcagagatgcctccctgagctagaaccctctgttccttccctgtgcctcctccctccctcccgactcacaccactagcctcaggggtctgagctccagctcctttgggcttcagctgccagtgtcctgagccccagggagagggggctggtgggtgcctaggcctgggcagtggatggccgtgaatgggtgcccacagtgtcaggcactgggcatgaggggttcctcccctccagctccctgtgcccccagggtcctgggaggagagacactggtggggataggccagccgcgcatcagactgtgaaccccacgaaggagcccattgtggcctaagaggctgccctcctgtgctcagccctgaggacagatgcctccttcctcttttccttcccaaagcaagcaagaggccgtggctgctgtgggaaatggtactgtacagctggctctacttccccatggccctgagcgagtggagtctgccacccaggatccccaaggcacttgagggggaaggattctgctggcctctgcgagtggtttcttgtgcactggcaccaagtgcgggtccggcagcttctgccccctgcagaaccggagagccagctaaggggtggggctgcgggggttccgtgtccacccccatacatttatttctgtaaataatgtgcactgaataaattgtacagccggcaaaaaaaaaaaaaaaaaaaaa

SEQ ID NO: 47 - protein, 607 aa, Homo sapiens
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGPTDRVKEGHSPPDDVDIVIKLDNTASTVKAIAIVIPCILALCLLVLVYTVFQFKRKGTPRHILYCKRSMQEWV

SEQ ID NO: 48 - DNA, 2052 bp, Homo sapiens
ggggagctcgtccatccattgaagcacagttcactatgatcttactcacattcagcactggaagacggttggatttcgtgcatcattcgggggtgtttttcttgcaaaccttgctttggattttatgtgctacagtctgcggaacggagcagtatttcaatgtggaggtttggttacaaaagtacggctaccttccaccgactgaccccagaatgtcagtgctgcgctctgcagagaccatgcagtctgccctagctgccatgcagcagttctatggcattaacatgacaggaaaagtggacagaaacacaattgactggatgaagaagccccgatgcggtgtacctgaccagacaagaggtagctccaaatttcatattcgtcgaaagcgatatgcattgacaggacagaaatggcagcacaagcacatcacttacagtataaagaacgtaactccaaaagtaggagaccctgagactcgtaaagctattcgccgtgcctttgatgtgtggcagaatgtaactcctctgacatttgaagaagttccctacagtgaattagaaaatggcaaacgtgatgtggatataaccattatttttgcatctggtttccatggggacagctctccctttgatggagagggaggatttttggcacatgcctacttccctggaccaggaattggaggagatacccattttgactcagatgagccatggacactaggaaatcctaatcatgatggaaatgacttatttcttgtagcagtccatgaactgggacatgctctgggattggagcattccaatgaccccactgccatcatggctccattttaccagtacatggaaacagacaacttcaaactacctaatgatgatttacagggcatccagaagatatatggtccacctgacaagattcctccacctacaagacctctaccgacagtgcccccacaccgctctattcctccggctgacccaaggaaaaatgacaggccaaaacctcctcggcctccaaccggcagaccctcctatcccggagccaaacccaacatctgtgatgggaactttaacactctagctattcttcgtcgtgagatgtttgttttcaaggaccagtggttttggcgagtgagaaacaacagggtgatggatggatacccaatgcaaattacttacttctggcggggcttgcctcctagtatcgatgcagtttatgaaaatagcgacgggaattttgtgttctttaaaggtaacaaatattgggtgttcaaggatacaactcttcaacctggttaccctcatgacttgataacccttggaagtggaattccccctcatggtattgattcagccatttggtgggaggacgtcgggaaaacctatttcttcaagggagacagatattggagatatagtgaagaaatgaaaacaatggaccctggctatcccaagccaatcacagtctggaaagggatccctgaatctcctcagggagcatttgtacacaaagaaaatggctttacgtatttctacaaaggaaaggagtattggaaattcaacaaccagatactcaaggtagaacctggatatccaagatccatcctcaaggattttatgggctgtgatggaccaacagacagagttaaagaaggacacagcccaccagatgatgtagacattgtcatcaaactggacaacacagccagcactgtgaaagccatagctattgtcattccctgcatcttggccttatgcctccttgtattggtttacactgtgttccagttcaagaggaaaggaacaccccgccacatactgtactgtaaacgctctatgcaagagtgggtgtgatgtagggttttttcttctttctttcttttgcaggagtttgtggtaacttgagattcaagacaagagctgttatgctgtttcctagctaggagcaggcttgtggcagcctgattcggggctgacctttcaaacccagagggttgctggtcctgcacatgagtggaaatacactcatggggaagcttccatgatg

SEQ ID NO: 49 - protein, 519 aa, Homo sapiens
MQQFGGLEATGILDEATLALMKTPRCSLPDLPVLTQARRRRQAPAPTKWNKRNLSWRVRTFPRDSPLGHDTVRALMYYALKVWSDIAPLNFHEVAGSTADIQIDFSKADHNDGYPFDGPGGTVAHAFFPGHHHTAGDTHFDDDEAWTFRSSDAHGMDLFAVAVHEFGHAIGLSHVAAAHSIMRPYYQGPVGDPLRYGLPYEDKVRVWQLYGVRESVSPTAQPEEPPLLPEPPDNRSSAPPRKDVPHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDSQADGSVAAGVDAAEGPRAPPGQHDQSRSEDGYEVCSCTSGASSPPGAPGPLVAATMLLLLPPLSPGALWTAAQALTL

SEQ ID NO: 50 - DNA, 2306 bp, Homo sapiens
aagagacaagaggtgccttgtgggcagatagggggctgggagggggcctgcccggaagcagtggtggcccgtggcaggcttctcactgggtaggaccgggccctctgttgcaccccctcaccctgctctctgccctcaggagtggctaagcaggttcggttacctgcccccggctgaccccacaacagggcagctgcagacgcaagaggagctgtctaaggccatcacagccatgcagcagtttggtggcctggaggccaccggcatcctggacgaggccaccctggccctgatgaaaaccccacgctgctccctgccagacctccctgtcctgacccaggctcgcaggagacgccaggctccagcccccaccaagtggaacaagaggaacctgtcgtggagggtccggacgttcccacgggactcaccactggggcacgacacggtgcgtgcactcatgtactacgccctcaaggtctggagcgacattgcgcccctgaacttccacgaggtggcgggcagcaccgccgacatccagatcgacttctccaaggccgaccataacgacggctaccccttcgacggccccggcggcaccgtggcccacgccttcttccccggccaccaccacaccgccggggacacccactttgacgatgacgaggcctggaccttccgctcctcggatgcccacgggatggacctgtttgcagtggctgtccacgagtttggccacgccattgggttaagccatgtggccgctgcacactccatcatgcggccgtactaccagggcccggtgggtgacccgctgcgctacgggctcccctacgaggacaaggtgcgcgtctggcagctgtacggtgtgcgggagtctgtgtctcccacggcgcagcccgaggagcctcccctgctgccggagcccccagacaaccggtccagcgccccgcccaggaaggacgtgccccacagatgcagcactcactttgacgcggtggcccagatccggggtgaagctttcttcttcaaaggcaagtacttctggcggctgacgcgggaccggcacctggtgtccctgcagccggcacagatgcaccgcttctggcggggcctgccgctgcacctggacagcgtggacgccgtgtacgagcgcaccagcgaccacaagatcgtcttctttaaaggagacaggtactgggtgttcaaggacaataacgtagaggaaggatacccgcgccccgtctccgacttcagcctcccgcctggcggcatcgacgctgccttctcctgggcccacaatgacaggacttatttctttaaggaccagctgtactggcgctacgatgaccacacgaggcacatggaccccggctaccccgcccagagccccctgtggaggggtgtccccagcacgctggacgacgccatgcgctggtccgacggtgcctcctacttcttccgtggccaggagtactggaaagtgctggatggcgagctggaggtggcacccgggtacccacagtccacggcccgggactggctggtgtgtggagactcacaggccgatggatctgtggctgcgggcgtggacgcggcagaggggccccgcgcccctccaggacaacatgaccagagccgctcggaggacggttacgaggtctgctcatgcacctctggggcatcctctcccccgggggccccaggcccactggtggctgccaccatgctgctgctgctgccgccactgtcaccaggcgccctgtggacagcggcccaggccctgacgctatgacacacagcgcgagcccatgagaggacagaggcggtgggacagcctggccacagagggcaaggactgtgccggagtccctgggggaggtgctggcgcgggatgaggacgggccaccctggcaccggaaggccagcagagggcacggcccgccagggctgggcaggctcaggtggcaaggacggagctgtcccctagtgagggactgtgttgactgacgagccgaggggtggccgctccagaagggtgcccagtcaggccgcaccgccgccagcctcctccggccctggagggagcatctcgggctgggggcccacccctctctgtgccggcgccaccaaccccacccacactgctgcctggtgctcccgccggcccacagggcctccgtccccaggtccccagtggggcagccctccccacagacgagccccccacatggtgccgcggcacgtcccccctgtgacgcgttccagaccaacatgacctctccctgctttgtagcggcccggaattc

SEQ ID NO: 51 - protein, 508 aa, Homo sapiens
MNCQQLWLGFLLPMTVSGRVLGLAEVAPVDYLSQYGYLQKPLEGSNNFKPEDITEALRAFQEASELPVSGQLDDATRARMRQPRCGLEDPFNQKTLKYLLLGRWRKKHLTFRILNLPSTLPPHTARAALRQAFQDWSNVAPLTFQEVQAGAADIRLSFHGRQSSYCSNTFDGPGRVLAHADIPELGSVHFDEDEFWTEGTYRGVNLRIIAAHEVGHALGLGHSRYSQALMAPVYEGYRPHFKLHPDDVAGIQALYGKKSPVIRDEEEEETELPTVPPVPTEPSPMPDPCSSELDAMMLGPRGKTYAFKGDYVWTVSDSGPGPLFRVSALWEGLPGNLDAAVYSPRTQWIHFFKGDKVWRYINFKMSPGFPKKLNRVEPNLDAALYWPLNQKVFLFKGSGYWQWDELARTDFSSYPKPIKGLFTGVPNQPSAAMSWQDGRVYFFKGKVYWRLNQQLRVEKGYPRNISHNWMHCRPRTIDTTPSGGNTTPSGTGITLDTTLSATETTFEY

SEQ ID NO: 52 - DNA, 1811 bp, Homo sapiens
gaattccgggagcccctctgcctagcactgctcccccaaggctcccagaaatctcaggtcagaggcacggacagcctctggagctctcgtctggtgggaccatgaactgccagcagctgtggctgggcttcctactccccatgacagtctcaggccgggtcctggggcttgcagaggtggcgcccgtggactacctgtcacaatatgggtacctacagaagcctctagaaggatctaataacttcaagccagaagatatcaccgaggctctgagagcttttcaggaagcatctgaacttccagtctcaggtcagctggatgatgccacaagggcccgcatgaggcagcctcgttgtggcctagaggatcccttcaaccagaagacccttaaatacctgttgctgggccgctggagaaagaagcacctgactttccgcatcttgaacctgccctccacccttccaccccacacagcccgggcagccctgcgtcaagccttccaggactggagcaatgtggctcccttgaccttccaagaggtgcaggctggtgcggctgacatccgcctctccttccatggccgccaaagctcgtactgttccaatacttttgatgggcctgggagagtcctggcccatgccgacatcccagagctgggcagtgtgcacttcgacgaagacgagttctggactgaggggacctaccgtggggtgaacctgcgcatcattgcagcccatgaagtgggccatgctctggggcttgggcactcccgatattcccaggccctcatggccccagtctacgagggctaccggccccactttaagctgcacccagatgatgtggcagggatccaggctctctatggcaagaagagtccagtgataagggatgaggaagaagaagagacagagctgcccactgtgcccccagtgcccacagaacccagtcccatgccagacccttgcagtagtgaactggatgccatgatgctggggccccgtgggaagacctatgctttcaagggggactatgtgtggactgtatcagattcaggaccgggccccttgttccgagtgtctgccctttgggaggggctccccggaaacctggatgctgctgtctactcgcctcgaacacaatggattcacttctttaagggagacaaggtgtggcgctacattaatttcaagatgtctcctggcttccccaagaagctgaatagggtagaacctaacctggatgcagctctctattggcctctcaaccaaaaggtgttcctctttaagggctccgggtactggcagtgggacgagctagcccgaactgacttcagcagctaccccaaaccaatcaagggtttgtttacgggagtgccaaaccagccctcggctgctatgagttggcaagatggccgagtctacttcttcaagggcaaagtctactggcgcctcaaccagcagcttcgagtagagaaaggctatcccagaaatatttcccacaactggatgcactgtcgtccccggactatagacactaccccatcaggtgggaataccactccctcaggtacgggcataaccttggataccactctctcagccacagaaaccacgtttgaatactgactgctcacccacagacacaatcttggacattaacccctgaggctccaccacccaccctttcatttcccccccagaagcctaaggcctaatagctgaatgaaatacctgtctgctcagtagaaccttgcaggtgctgtagcaggcgcaagaccgtagatctcaggcctctaacacttccaactc

SEQ ID NO: 53 - protein, 483 aa, Homo sapiens [sequence omitted]

SEQ ID NO: 54 - DNA, 1674 bp, Homo sapiens
ctactgtgaggggatgaaggtgctccctgcatctggccttgctgtcttcctcatcatggctttgaagttttccactgcagccccctccctagttgcagcctcccccaggacctggaggaacaactaccgcctcgcacaggcgtatcttgacaaatattacacaaataaagaaggacaccagattggtgagatggttgcaagaggaagcaattccatgataaggaagattaaggagctacaagcgttctttggcctccaagtcaccgggaagttagaccagaccacaatgaacgtgatcaagaagcctcgctgtggagttcctgatgtggccaattatcgcctcttccctggtgaacccaaatggaaaaaaaatactttgacatacagaatatctaaatacacaccttccatgagttctgtcgaggtggacaaagcagtggagatggccttgcaggcctggagtagcgccgtccctctgagctttgtcagaataaactcaggagaagcggatattatgatatcttttgaaaatggagatcacggggattcctatccattcgatgggcctcgggggactctagcccatgcatttgctcctggagaaggcctgggaggagatacacatttcgacaatcctgagaagtggactatgggaacgaatggttttaatttgtttaccgttgctgctcatgaatttggccatgccctgggcctggcccattccacagacccatcagcactgatgtacccaacttataagtacaagaatccctatggattccacctccccaaagatgatgtgaaagggatccaggcattatacggacctcggaaagtattcctggggaagcccactctgccccatgccccccatcacaagccatccatccctgacctctgtgactccagctcatcctttgacgctgtgacaatgctggggaaggagctcctgctcttcaaggaccggattttctggagacggcaggttcacttgcggacaggaattcggcccagcactattaccagctccttcccccagctcatgtccaatgtggatgcagcttacgaagtggctgagaggggcactgcttacttcttcaaaggtccccactactggataacaagaggattccaaatgcaaggtcctcctcggactatttatgactttggatttccaaggcacgtgcagcaaatagatgctgctgtctacctcagggagccacagaagacccttttctttgtgggagatgaatactacagctacgacgaaaggaaaaggaaaatggaaaaagactatccaaagaatactgaagaagaattttcaggagtaaatggccaaatcgatgctgctgtagaattaaatggctacatttacttcttttcaggaccaaaaacatacaagtatgacacagagaaggaagatgtggttagtgtggtgaaatctagttcctggattggttgctaaatagaaaagcctagtcttctcaagcaatgaggatgactacaagcagcctctaactggatcttaaggactaaagcagaatgtaggagagggattcttccaaaggccttcaaatcaaattagaattcactgagaataataatacttccaatttttttcatagttgtataatcagaatttcaatccacattagaaaagtttttatatgggca

SEQ ID NO: 55 - protein, 390 aa, Homo sapiens
MGRGARVPSEAPGAGVERRWLGAALVALCLLPALVLLARLGAPAVPAWSAAQGDVAALGLSAVPPTRVPGPLAPRRRRYTLTPARLRWDHFNLTYRILSFPRNLLSPRETRRALAAAFRMWSDVSPFSFREVAPEQPSDLRIGFYPINHTDCLVSALHHCFDGPTGELAHAFFPPHGGIHFDDSEYWVLGPTRYSWKKGVWLTDLVHVAAHEIGHALGLMHSQHGRALMHLNATLRGWKALSQDELWGLHRLYGCLDRLFVCASWARRGFCDARRRLMKRLCPSSCDFCYEFPFPTVATTPPPPRTKTRLVPEGRNVTFRCGQKILHKKGKVYWYKDQEPLEFSYPGYLALGEAHLSIIANAVNEGTYTCVVRRQQRVLTTYSWRVRVRG

SEQ ID NO: 56 - DNA, 1212 bp, Homo sapiens
agccctgagccccatagcaagtctgccatgggccgcggggcccgtgtcccctcggaggccccgggggcaggcgtcgagcgccgctggcttggagccgcgctggtcgccctgtgcctcctccccgcgctggtgctgctggcccggctgggggccccggcggtgccggcctggagcgcagcgcagggagacgtcgctgcgctgggcctctcggcggtcccccccacccgggtcccgggcccactggccccccgcagacgccgctacacgctgactccagccaggctgcgctgggaccacttcaacctcacctacaggatcctctccttcccgcggaacctgctgagcccgcgggagacgcggcgggccctagctgccgccttccgcatgtggagcgacgtgtcccccttcagcttccgcgaggtggcccccgagcagcccagcgacctccggataggcttctacccgatcaaccacacggactgcctggtctccgcgctgcaccactgcttcgacggccccacgggggagctggcccacgccttcttccccccgcacggcggcatccacttcgacgacagcgagtactgggtcctgggccccacgcgctacagctggaagaaaggcgtgtggctcacggacctggtgcacgtggcggcccacgagatcggccacgcgctgggcctgatgcactcacaacacggccgggcgctcatgcacctgaacgccacgctgcgcggctggaaggcgttgtcccaggacgagctgtgggggctgcaccggctctacggatgcctcgacaggctgttcgtgtgcgcgtcctgggcgcggaggggcttctgcgacgctcgccggcggctcatgaagaggctctgccccagcagctgcgacttctgctacgaattccccttccccacggtggccaccaccccaccgccccccaggaccaaaaccaggctggtgcccgagggcaggaacgtgaccttccgctgcggccagaagatcctccacaagaaagggaaagtgtactggtacaaggaccaggagcccctggagttctcctaccccggctacctggccctgggcgaggcgcacctgagcatcatcgccaacgccgtcaatgagggcacctacacctgcgtggtgcgccgccagcagcgcgtgctgaccacctactcctggcgagtccgtgtgcggggctgagcccggctgata

SEQ ID NO: 57 - protein, 645 aa, Homo sapiens
MPRSRGGRAAPGPPPPPPPPGQAPRWSRWRVPGRLLLLLLPALCCLPGAARAAAAAAGAGNRAAVAVAVARADEAEAPFAGQNWLKSYGYLLPYDSRASALHSAKALQSAVSTMQQFYGIPVTGVLDQTTIEWMKKPRCGVPDHPHLSRRRRNKRYALTGQKWRQKHITYSIHNYTPKVGELDTRKAIRQAFDVWQKVTPLTFEEVPYHEIKSDRKEADIMIFFASGFHGDSSPFDGEGGFLAHAYFPGPGIGGDTHFDSDEPWTLGNANHDGNDLFLVAVHELGHALGLEHSSDPSAIMAPFYQYMETHNFKLPQDDLQGIQKIYGPPAEPLEPTRPLPTLPVRRIHSPSERKHERQPRPPRPPLGDRPSTPGTKPNICDGNFNTVALFRGEMFVFKDRWFWRLRNNRVQEGYPMQIEQFWKGLPARIDAAYERADGRFVFFKGDKYWVFKEVTVEPGYPHSLGELGSCLPREGIDTALRWEPVGKTYFFKGERYWRYSEERRATDPGYPKPITVWKGIPQAPQGAFISKEGYYTYFYKGRDYWKFDNQKLSVEPGYPRNILRDWMGCNQKEVERRKERRLPQDDVDIMVTINDVPGSVNAVAVVIPCILSLCILVLVYTIFQFKNKTGPQPVTYYKRPVQEWV

SEQ ID NO: 58 - DNA, 2118 bp, Homo sapiens
atgccgaggagccggggcggccgcgccgcgccggggccgccgccgccgccgccgccgccgggccaggccccgcgctggagccgctggcgggtccctgggcggctgctgctgctgctgctgcccgcgctctgctgcctcccgggcgccgcgcgggcggcggcggcggcggcgggggcagggaaccgggcagcggtggcggtggcggtggcgcgggcggacgaggcggaggcgcccttcgccgggcagaactggttaaagtcctatggctatctgcttccctatgactcacgggcatctgcgctgcactcagcgaaggccttgcagtcggcagtctccactatgcagcagttttacgggatcccggtcaccggtgtgttggatcagacaacgatcgagtggatgaagaaaccccgatgtggtgtccctgatcacccccacttaagccgtaggcggagaaacaagcgctatgccctgactggacagaagtggaggcaaaaacacatcacctacagcattcacaactataccccaaaagtgggtgagctagacacgcggaaagctattcgccaggctttcgatgtgtggcagaaggtgaccccactgacctttgaagaggtgccataccatgagatcaaaagtgaccggaaggaggcagacatcatgatcttttttgcttctggtttccatggcgacagctccccatttgatggagaagggggattcctggcccatgcctacttccctggcccagggattggaggagacacccactttgactccgatgagccatggacgctaggaaatgccaaccatgacgggaacgacctcttcctggtggctgtgcatgagctgggccacgcgctgggactggagcactccagcgaccccagcgccatcatggcgcccttctaccagtacatggagacgcacaacttcaagctgccccaggacgatctccagggcatccagaagatctatggacccccagccgagcctctggagcccacaaggccactccctacactccccgtccgcaggatccactcaccatcggagaggaaacacgagcgccagcccaggccccctcggccgcccctcggggaccggccatccacaccaggcaccaaacccaacatctgtgacggcaacttcaacacagtggccctcttccggggcgagatgtttgtctttaaggatcgctggttctggcgtctgcgcaataaccgagtgcaggagggctaccccatgcagatcgagcagttctggaagggcctgcctgcccgcatcgacgcagcctatgaaagggccgatgggagatttgtcttcttcaaaggtgacaagtattgggtgtttaaggaggtgacggtggagcctgggtacccccacagcctgggggagctgggcagctgtttgccccgtgaaggcattgacacagctctgcgctgggaacctgtgggcaagacctactttttcaaaggcgagcggtactggcgctacagcgaggagcggcgggccacggaccctggctaccctaagcccatcaccgtgtggaagggcattccacaggctccccaaggagccttcatcagcaaggaaggatattacacctatttctacaagggccgggactactggaagtttgacaaccagaaactgagcgtggagccaggctacccgcgcaacatcctgcgtgactggatgggctgcaaccagaaggaggtggagcggcggaaggagcggcggctgccccaggacgacgtggacatcatggtgaccatcaacgatgtgccgggctccgtgaacgccgtggccgtggtcatcccctgcatcctgtccctctgcatcctggtgctggtctacaccatcttccagttcaagaacaagacaggccctcagcctgtcacctactataagcggccagtccaggaatgggtgtgagcagcccagagccctctctatccacttggtctggccagccaggcccttcctcaccagggtctgaggggcagctctggccagtgctcaccagggccagcagggctaggctggggtcgtacagctgaagtggtgggtgcattggcctaggctgagcgtggggcagggaattatgggggctgt

SEQ ID NO: 59 - protein, 440 aa, Homo sapiens
MDPGTVATMRKPRCSLPDVLGVAGLVRRRRRYALSGSVWKKRTLTWRVRSFPQSSQLSQETVRVLMSYALMAWGMESGLTFHEVDSPQGQEPDILIDFARAFHQDSYPFDGLGGTLAHAFFPGEHPISGDTHFDDEETWTFGSKADGEGTDLFAVAVHEFGHALGLGHSSAPNSIMRPFYQGPVGDPDKYRLSQDDRDGLQQLPCLGENKPPSLLTSPFLPSPSFPIPDRCEGNFDAIANIRGETFFFKGGPWFWRLQPSGQLVSPRPARLHRFWEGLPAQVRVVQAAYARHRDGRILLFSGPQFWVFQDRQLEGGARPLTELGLPPGEEVDAVFSWPQNGKTYLVRGRQYWRYDEAAARPDPGYPRDLSLWEGAPPSPDDVTVSNAGGERGDLRVTGPGGGERDVGNGDMEATLRGWGSLGIRERRGGEGPGLKLCSSR

SEQ ID NO: 60 - DNA, 1323 bp, Homo sapiens
atggacccagggacagtggccaccatgcgtaagccccgctgctccctgcctgacgtgctgggggtggcggggctggtcaggcggcgtcgccggtacgctctgagcggcagcgtgtggaagaagcgaaccctgacatggagggtacgttccttcccccagagctcccagctgagccaggagaccgtgcgggtcctcatgagctatgccctgatggcctggggcatggagtcaggcctcacatttcatgaggtggattccccccagggccaggagcccgacatcctcatcgactttgcccgcgccttccaccaggacagctaccccttcgacgggttggggggcaccctagcccatgccttcttccctggggagcaccccatctccggggacactcactttgacgatgaggagacctggacttttgggtcaaaagcagacggcgaggggaccgacctgtttgccgtggctgtccatgagtttggccacgccctgggcctgggccactcctcagcccccaactccattatgaggcccttctaccagggtccggtgggcgaccctgacaagtaccgcctgtctcaggatgaccgcgatggcctgcagcaactcccctgccttggagaaaacaaacccccctctctactcacctctcctttcctccccagcccatccttccccatccctgatcgatgtgagggcaattttgacgccatcgccaacatccgaggggaaactttcttcttcaaaggaggcccctggttctggcgcctccagccctccggacagctggtgtccccgcgacccgcacggctgcaccgcttctgggaggggctgcccgcccaagtgagggtggtgcaggccgcctatgctcggcaccgagacggccgaatcctcctctttagcgggccccagttctgggtgttccaggaccggcagctggagggcggggcgcggccgctcacggagctggggctgcccccgggagaggaggtggacgccgtgttctcgtggccacagaacgggaagacctacctggtccgcggccggcagtactggcgctacgacgaggcggcggcgcgcccggaccccggctaccctcgcgacctgagcctctgggaaggcgcgcccccctcccctgacgatgtcaccgtcagcaacgcaggtggggagcgcggtgacctgcgggttactgggcctgggggtggggagagggatgtggggaatggggacatggaggccaccctgcggggatgggggtccttgggcatcagggagcggcggggcggggagggaccgggactcaagctctgctcctccaggtga

(19) United States
(12) Patent Application Publication
(10) Pub. No.: US 2005/0026836 A1
(43) Pub. Date: Feb. 3, 2005

(54) Composition for the treatment of damaged tissue

(76) Inventors: Kevin Neil Dack, County of Kent (GB); Michael John Davies, County of Kent (GB); Paul Vincent Fish, County of Kent (GB); Jonathan Paul Huggins, Sandwich (GB); Fraser Stuart McIntosh, County of Kent (GB); Nicholas Laurence Occleston, County of Kent (GB)

Correspondence Address: WARNER-LAMBERT COMPANY, 2800 PLYMOUTH RD, ANN ARBOR, MI, 48105, US

(21) Appl. No.: 10/901,417

(22) Filed: Jul. 28, 2004

Related U.S. Application Data
(63) Continuation of application No. 10/131,985, filed on Apr. 25, 2002.
Application No. 10/131,985 is a continuation of application No. 09/725,295, filed on Nov. 29, 2000, now abandoned.
(60) Provisional application No. 60/186,426, filed on Mar. 2, 2000.

(30) Foreign Application Priority Data
Dec. 29, 1999 (GB) 9930768.8

(51) Int. Cl. A61K 38/18

(52) U.S. Cl. 514/12

(57) ABSTRACT

A pharmaceutical for use in damaged tissue, such as wound, treatment (e.g. healing) is described. The pharmaceutical comprising a composition which comprises: (a) a growth factor; and (b) an inhibitor agent; and optionally (c) a pharmaceutically acceptable carrier, diluent or excipient; wherein the inhibitor agent can inhibit the action of at least one specific adverse protein (e.g. a specific protease) that is upregulated in a damaged tissue, such as a wound, environment.

### FIELD OF INVENTION

The present invention relates to a composition, in particular a pharmaceutical composition. The present invention also relates to uses of that composition—in particular in the treatment of damaged tissue.

### BACKGROUND ART

It is desirable to be able to treat damaged tissue, such as in wounds, more in particular in chronic wounds. Examples of chronic wounds include chronic dermal ulceration.

Chronic dermal ulcers are a major cause of morbidity in the ageing population, and represent a significant economic burden on healthcare systems. Recent figures for chronic dermal ulcers, including pressure sores, diabetic and venous ulcers, indicate a total of about 3.75 million and 12 million patients in the US and world-wide, respectively (Wound Healing Technological Innovations and Market Overview (1998) Technology Catalysts International Corporation, VA, USA). Of these patients, approximately 70% are classified as moderate to severe. Despite recent advances in their treatment, the healing of these ulcers remains slow (typically 16 weeks for a venous ulcer with best care) and agents which are efficacious in reducing the time to closure will bring medical and commercial benefit.

The present invention seeks to overcome these problems.

### SUMMARY ASPECTS OF THE PRESENT INVENTION

In accordance with the present invention, damaged tissue, such as wounds (in particular chronic wounds), can be treated more effectively if a combination of a growth factor and an inhibitor agent is used. The inhibitor agent used is, or is derivable from or is based on, a protease inhibitor. In more detail, the inhibitor agent inhibits the action of specific proteins that are upregulated in a wound environment wherein those proteins have an adverse effect in the wound environment. Here, typically the adverse effect is a deleterious effect on wound healing. Typically these adverse proteins are adverse proteases that are upregulated in a wound environment. Hence, the inhibitor agent is a specific inhibitor agent.

Thus, one aspect of the present invention concerns a composition for use in or as a pharmaceutical (otherwise called a medicament), wherein said composition comprises an inhibitor agent that inhibits the action of at least one specific protease protein that is upregulated in a wound environment.

In one preferred aspect, the present invention concerns a composition for use in or as a pharmaceutical (otherwise called a medicament), wherein said composition comprises an inhibitor agent that inhibits the action of a specific protease protein that is upregulated in a wound environment.

The combination of the protease inhibitor and the growth factor results in a beneficial additive effect, which in some cases is synergistic.

We believe that, in use, the protease inhibitor agent of the present invention protects the growth factor in the damaged tissue environment and to such an extent that the degradation of the growth factor is hindered, delayed, reduced or even eliminated.

The use of an inhibitor agent that inhibits the action of one or more specific adverse proteins—in particular one or more specific proteases—that are upregulated in a wound environment is in direct contrast to the teachings of workers who have used non-selective inhibitors. By way of example, reference may be made to Kiyohara Yoshifumi et al (Database Biosis Database Accession No. PREV199497178695 XP002139251 reporting on Biological & Pharmaceutical Bulletin 1993 vol 16 pages 1146-1149); Wlaschek et al (British Journal of Dermatology 1997 137(4) page 646); Witte et al (Surgery (St Louis) 1998 vol 124 (2) pages 464-470); Ryou et al (Arch Pharmacal Res 1997 vol 20 (1) pages 34-38); Singer et al (New England Journal of Medicine Sep. 2, 1999 vol 341 (10) pages 738-746); Chen Chin et al (Wound Repair and Regeneration vol 7 (6) pages 486-494); and U.S. Pat. No. 5,290,762.

### DETAILED ASPECTS OF THE PRESENT INVENTION

According to one aspect, the present invention provides a pharmaceutical for use (or when in use) in the treatment (e.g. healing) of damaged tissue (such as damaged tissue in a wound); the pharmaceutical comprising a composition, which composition comprises: (a) a growth factor; and (b) an inhibitor agent; and optionally (c) a pharmaceutically acceptable carrier, diluent or excipient; wherein the inhibitor agent can inhibit the action of at least one specific adverse protein (e.g. a specific protease) that is upregulated in a damaged tissue environment.

In accordance with the present invention, the growth factor is sometimes referred to as “component (a); the inhibitor agent is sometimes referred to as “component (b)”; and the pharmaceutically acceptable carrier, diluent or excipient is sometimes referred to as “component (c)”.

Typically, for topical mixtures or locally injected mixtures, the relative ratio of inhibitor agent to growth factor may be between 1000:1 and 1:1 (on a mg:mg or a %:% basis).

Typically, for a systemically administered inhibitor agent with a topical or locally injected growth factor, the relative ratio of inhibitor agent to growth factor may be between 10,000:1 and 10:1 (on a mg:mg basis).

According to another aspect, the present invention provides a composition according to the present invention for use in medicine.

According to another aspect, the present invention provides the use of a composition according to the present invention in the manufacture of a pharmaceutical to treat damaged tissue, such as wounds.

According to another aspect, the present invention provides the use of a composition according to the present invention in the manufacture of a pharmaceutical to treat chronic damaged tissue, such as chronic wounds.

According to another aspect, the present invention provides the use of a composition according to the present invention in the manufacture of a pharmaceutical to treat a chronic dermal ulcer.

According to another aspect, the present invention provides a method of therapy, said method comprising administering to a subject a composition according to the present invention and in an amount to treat (e.g. heal) damaged tissue, such as a wound.

According to another aspect, the present invention provides a process for preparing a composition according to the present invention; said process comprising the steps of admixing one or more of said agent(s) according to the present invention with a growth factor and optionally a pharmaceutically acceptable carrier, diluent or excipient.

According to another aspect, the present invention provides a process; said process comprising the steps of: (a) admixing one or more of said agent(s) according to the present invention with a growth factor and optionally a pharmaceutically acceptable carrier, diluent or excipient; (ii) administering said composition to a subject in need of same.

According to another aspect, the present invention provides performing an assay to identify one or more agents that are capable of acting as an inhibitor agent according to the present invention.

According to another aspect, the present invention provides a process for preparing a composition according to the present invention; said process comprising the steps of: (i) performing an assay to identify one or more agents that are capable of acting as an inhibitor agent according to the present invention; (ii) admixing one or more of said agent(s) with a growth factor and optionally a pharmaceutically acceptable carrier, diluent or excipient.

According to another aspect, the present invention provides a process; said process comprising the steps of: (i) performing an assay to identify one or more agents that are capable of acting as an inhibitor agent according to the present invention; (ii) admixing one or more of said agent(s) with a growth factor and optionally a pharmaceutically acceptable carrier, diluent or excipient; (iii) administering said composition to a subject in need of same.

It is to be understood that components (a) and (b) may be present in the same admixture for administration to a subject or they may be administered to a subject sequentially or simultaneously, and in doing so they may be applied by similar or different techniques. Thus, the components may be administered together, such as in the same admixture. In the alternative, one of the components may be administered orally, systemically, topically or by injection and the other of the components may be taken by a similar route (e.g. one of orally, systemically, topically, or by injection) or by a different route (e.g. a different one of orally, systemically, topically or by injection). In one preferred embodiment of the present invention, one component is applied topically and the other component is applied systemically. In another preferred embodiment of the present invention, one component is applied topically and the other component is applied topically.

Thus, according to one aspect, the present invention provides a pack for use in the treatment (e.g. healing) of damaged tissue, such as a wound; the pack comprising at least two compartments; wherein first of said compartments houses a growth factor; and wherein second of said compartments houses an inhibitor agent, wherein the inhibitor agent can inhibit the action of at least one specific adverse protein (e.g. a specific protease) that is upregulated in a damaged tissue, such as a wound, environment. In the pack of the present invention, the growth factor and/or the inhibitor agent may be admixed with a pharmaceutically acceptable carrier, diluent or excipient. In addition, or in the alternative, the pack of the present invention comprises a third compartment, which third compartment houses a pharmaceutically acceptable carrier, diluent or excipient.

With the present invention, such as the pack of the present invention, the growth factor and the inhibitor agent may be in different forms. By way of example, one may be a solution or tablet and the other may be a cream. In one preferred embodiment of the present invention, one component of the pack is to be applied topically and the other component of the pack is to be applied systemically. It is to be understood that the pack could contain extra compartments.

According to one aspect of the present invention, there is provided a process for preparing a pharmaceutical for use in damaged tissue, such as wound, treatment (e.g. healing); the process comprising forming a composition by admixing (a) a growth factor with (b) an inhibitor agent; and optionally with (c) a pharmaceutically acceptable carrier, diluent or excipient; wherein the inhibitor agent can inhibit the action of at least one specific adverse protein (e.g. a specific protease) that is upregulated in a damaged tissue, such as a wound, environment.

According to one aspect of the present invention, there is provided the use of a growth factor according to the present invention in the manufacture of a pharmaceutical to treat a subject that is being treated with an inhibitor agent according to the present invention.

According to one aspect of the present invention, there is provided the use of an inhibitor agent according to the present invention in the manufacture of a pharmaceutical to treat a subject that is being treated with a growth factor according to the present invention.

According to one aspect of the present invention, there is provided a method of therapy, said method comprising administering to a subject a composition according to the present invention and in an amount to treat (e.g. heal) damaged tissue, such as a wound. Here, all or some (preferably all) of said growth factor according to the present invention may be administered by a different route than all or some (preferably all) of said inhibitor agent according to the present invention. However, preferably at least the inhibitor and/or the growth factor is applied topically. In one preferred aspect, both the inhibitor and the growth factor are applied topically. In another preferred aspect, the inhibitor is applied orally and the growth factor is applied topically.

According to one aspect of the present invention, there is provided the use of a composition according to the present invention in the manufacture of a pharmaceutical to treat chronic damaged tissue, such as chronic damaged wounds. Here, all or some (preferably all) of said growth factor according to the present invention may be administered by a different route than all or some (preferably all) of said inhibitor agent according to the present invention. However, preferably at least the inhibitor and/or the growth factor is applied topically. In a preferred aspect, both the inhibitor and the growth factor are applied topically. In another preferred aspect, the inhibitor is applied orally and the growth factor is applied topically.

According to one aspect of the present invention, there is provided the use of a growth factor according to the present invention in the manufacture of a pharmaceutical to treat a subject that is being treated with an inhibitor agent according to the present invention. Here, all or some (preferably all) of said growth factor according to the present invention may be administered by a different route than all or some (preferably all) of said inhibitor agent according to the present invention. However, preferably at least the inhibitor and/or the growth factor is applied topically. In a preferred aspect, both the inhibitor and the growth factor are applied topically. In another preferred aspect, the inhibitor is applied orally and the growth factor is applied topically.

According to one aspect of the present invention, there is provided the use of an inhibitor agent according to the present invention in the manufacture of a pharmaceutical to treat a subject that is being treated with a growth factor according to the present invention. Here, all or some (preferably all) of said growth factor according to the present invention may be administered by a different route than all or some (preferably all) of said inhibitor agent according to the present invention. However, preferably at least the inhibitor and/or the growth factor is applied topically. In a preferred aspect, both the inhibitor and the growth factor are applied topically. In another preferred aspect, the inhibitor is applied orally and the growth factor is applied topically.

According to one aspect of the present invention there is provided a pharmaceutical comprising:
(a) a growth factor;
(b) an i:UPA and/or an iMMP; and optionally
(c) a pharmaceutically acceptable carrier, diluent or excipient;
   wherein the iUPA and/or the iMMP can inhibit the action of at least one specific adverse protein (e.g. a specific protease) that is upregulated in a damaged tissue, such as a wound, environment.

With this embodiment, the growth factor may be endogeneous growth factor.

Here, all or some (preferably all) of said growth factor according to the present invention may be administered by a different route than all or some (preferably all) of said inhibitor agent according to the present invention. However, preferably at least the inhibitor and/or the growth factor is applied topically. In a preferred aspect, both the inhibitor and the growth factor are applied topically. In another preferred aspect, the inhibitor is applied orally and the growth factor is applied topically.

According to one aspect of the present invention there is provided the use of a pharmaceutical comprising:
(a) a growth factor;
(b) an i:UPA and/or an iMMP; and optionally
(c) a pharmaceutically acceptable carrier, diluent or excipient;
   wherein the iUPA and/or the iMMP can inhibit the action of at least one specific adverse protein (e.g. a specific protease) that is upregulated in a damaged tissue, such as a wound, environment to treat damaged tissue, such as wound.

With this embodiment, the growth factor may be endogeneous growth factor.

Here, all or some (preferably all) of said growth factor according to the present invention may be administered by a different route than all or some (preferably all) of said inhibitor agent according to the present invention. However, preferably at least the inhibitor and/or the growth factor is applied topically. In a preferred aspect, both the inhibitor and the growth factor are applied topically. In another preferred aspect, the inhibitor is applied orally and the growth factor is applied topically.

According to one aspect of the present invention there is provided a pharmaceutical composition comprising:
(i) an i:UPA
(ii) an iMMP; and optionally
(iii) a pharmaceutically acceptable carrier, diluent or excipient;
   wherein the iUPA and/or the iMMP can inhibit the action of at least one specific adverse protein (e.g. a specific protease) that is upregulated in a damaged tissue, such as a wound, environment.

For ease of reference, these and further aspects of the present invention are now discussed under appropriate section headings. However, the teachings under each section are not necessarily limited to each particular section.

Preferable Aspects

Preferably said growth factor is selected from one or more of: PDGF (platelet derived growth factor), FGF (fibroblast growth factor), CTGF (connective tissue derived growth factor), KGF (keratinocyte-derived growth factor), TGF (transforming growth factor), CSF (colony stimulating factor), VEGF (vascular endothelial growth factor), EGF (epidermal growth factor), Chrysalin, or active variants, homologues, derivatives or fragments of any thereof.

Preferably said growth factor is selected from one or more of VEGF, EGF, PDGF, FGF, CTGF-like, KGF-2, TGF-β, GM-CSF (granulocyte/macrophage stimulating factor), Chrysalin, or active variants, homologues, derivatives or fragments thereof.

Preferably said growth factor is at least PDGF, or an active variant, homologue, derivative or fragment thereof. Examples of fragments include the PDGF A-chain and the PDGF B-chain.

Typically, the protein that is upregulated in a damaged tissue, such as a wound environment, is a protease.

Preferably said inhibitor agent is an inhibitor of urokinase-type plasminogen activator (otherwise referred to as an I:uPA—sometimes written as i:UPA or as I:UPA) and/or an inhibitor of a matrix metalloproteinase (otherwise referred to as an I:MMP—sometimes written as i:MMP).

Preferably said damaged tissue is a wound.

Preferably said wound is a chronic wound.

Preferably said wound is a dermal ulcer.

Preferably said route(s) of administration is(are) selected from at least one or more of: oral administration, injection (such as direct injection), topically, inhalation, parenteral administration, mucosal administration, intramuscular administration, intravenous administration, subcutaneous administration, intraocular administration or transdermal administration.

Preferably said route(s) of administration is(are) oral administration and/or topical administration.

Preferably at least a part (preferably all) of said inhibitor is administered (delivered) by topical administration and so is formulated for such an administration route.

Preferably at least a part (preferably all) of said growth factor is administered topically and so is formulated for such an administration route.

Preferably, the inhibitor is at least an i:UPA. In an alternative embodiment, or in addition, preferably the inhibitor is at least an i:MMP; wherein said MMP is MMP 3 and/or MMP 13.

Inhibit the Action of at Least One Specific Adverse Protein (e.g. a Specific Protease) that is Upregulated in a Damaged Tissue

The term “inhibit the action of at least one specific adverse protein (e.g. a specific protease) that is upregulated in a damaged tissue” means that the inhibitor agent of the present invention does not have an activity profile over a broad number of proteins Instead, the inhibitor agent is capable of substantially selectively acting on a specific adverse protein (e.g. a specific protease) that is upregulated in a damaged tissue. In some circumstances, the inhibitor agent may act on a few specific proteins that are upregulated in a damaged tissue. However, preferably, the inhibitor agent is capable of selectively acting on one specific adverse protein (e.g. a specific protease) that is upregulated in a damaged tissue. Alternatively expressed in a highly preferred aspect, the inhibitor agent of the present invention is an agent that limits the specific proteolytic degradation effect(s) of at least one specific adverse protease that has a deleterious effect on wound healing.

Preferably, the inhibitor agent is selective—for example being at least about 50-fold, more preferably at least about 75-fold, more preferably at least about 100-fold, in terms of relative Ki measured using purified enzymes—over other proteases found in the damaged tissue, such as wound, environment. Depending on the selection of inhibitor agent, examples of other protease proteins may include one or more of: MMPs, tPA, plasmin and neutrophil elastase, some of which have a beneficial effect on would healing.

For some applications, preferably the agent has a Kᵢ value against a particular desired protein target of less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the agent has at least about a 100 fold selectivity to a particular desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target, preferably at least about a 400 fold selectivity to the desired target, preferably at least about a 450 fold selectivity to the desired target, preferably at least about a 500 fold selectivity to the desired target, preferably at least about a 600 fold selectivity to the desired target, preferably at least about a 700 fold selectivity to the desired target, preferably at least about an 800 fold selectivity to the desired target, preferably at least about a 900 fold selectivity to the desired target, preferably at least about a 1000 fold selectivity to the desired target.

For some applications, preferably the inhibitor agent of the present invention has a K₁ value of less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some embodiments of the present invention, preferably the agents of the present invention have a log D of −2 to +4, more preferably −1 to +2. The log D can be determined by standard procedures known in the art such as described in J. Pharm. Pharmacol. 1990, 42:144.

In addition, or in the alternative, for some embodiments preferably the agents of the present invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci 79, 7, p595-600 (1990), and Pharm. Res. vol 14, no. 6 (1997).

Treatment

It is to be appreciated that all references herein to treatment include one or more of curative, palliative and prophylactic treatment. Preferably, the term treatment includes at least curative treatment and/or palliative treatment.

The treatment may be of one or more of chronic dermal ulceration, diabetic ulcers, decubitus ulcers (or pressure sores), venous insufficiency ulcers, venous stasis ulcers, burns, corneal ulceration or melts.

The treatment may be for treating conditions associated with impaired damaged tissue, such as wound, healing, where impairment is due to diabetes, age, cancer or its treatment (including radiotherapy), neuropathy, nutritional deficiency or chronic disease.

Amino Acid Sequence

Aspects of the present invention concern the use of amino acid sequences. These amino acid sequences may be a component of the composition of the present invention—such as the growth factor component. In another embodiment, the amino acid sequences may be used as a target to identify suitable inhibitor agents for use in the composition of the present invention. In another embodiment, the amino acid sequences may be used as a target to verify that an agent may be used as an inhibitor agent in the composition of the present invention.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”. In some instances, the term protein is a protease.

The amino acid sequence may be prepared isolated from a suitable source, or it may be made synthetically or it may be prepared by use of recombinant DNA techniques.

In one aspect, the present invention provides an amino acid sequence that is used as a component of the composition of the present invention.

In another aspect, the present invention provides an amino acid sequence that is capable of acting as a target in an assay for the identification of one or more agents and/or derivatives thereof capable of acting as an inhibitor of said amino acid.

Nucleotide Sequence

Aspects of the present invention concern the use of nucleotide sequences. These nucleotide sequences may be used to express amino acid sequences that may be used as a component of the composition of the present invention—such as the growth factor component. In another embodiment, the nucleotide sequences may be used as a target to identify suitable inhibitor agents for use in the composition of the present invention. In another embodiment, the nucleotide sequences may be used as a target to verify that an agent may be used as an inhibitor agent in the composition of the present invention.

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form.

In one aspect, the present invention provides a nucleotide sequence encoding a substance capable of acting as a target in an assay (such as a yeast two hybrid assay) for the identification of one or more agents and/or derivatives thereof capable of acting as an inhibitor of said nucleotide sequence (or the amino acid encoded thereby).

Variants/Homologues/Derivatives

In addition to the specific amino acid sequences and nucleotide sequences mentioned herein, the present invention also encompasses the use of variants, homologues and derivatives of any thereof. Here, the term “homologue” means an entity having a certain homology with the subject amino acid sequences and the subject nucleotide sequences. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned. with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acids^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell DC, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used a probe to identify similar coding sequences in other organisms etc.

Hybridisation

The present invention also encompasses the use of nucleotide sequences that are capable of hybridising to the sequences presented herein, or any derivative, fragment or derivative thereof—such as if the agent is an anti-sense sequence.

The term “hybridization” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hydridising to the nucleotide sequences presented herein.

Preferably, the term “variant” encompasses sequences that are complementary to sequences that are capable of hydridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hydridising under high stringent conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

Regulatory Sequences

In some applications, the polynucleotide for use in the present invention is operably linked to a regulatory sequence which is capable of providing for the expression of the coding sequence, such as by the chosen host cell. By way of example, the present invention covers a vector comprising the polynucleotide of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the polynucleotide encoding the polypeptide of the present invention may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of the polypeptide of the present invention

Preferably, the nucleotide sequence of the present invention may be operably linked to at least a promoter.

Aside from the promoter native to the gene encoding the polypeptide of the present invention, other promoters may be used to direct expression of the polypeptide of the present invention. The promoter may be selected for its efficiency in directing the expression of the polypeptide of the present invention in the desired expression host.

In another embodiment, a constitutive promoter may be selected to direct the expression of the desired polypeptide of the present invention. Such an expression construct may provide additional advantages since it circumvents the need to culture the expression hosts on a medium containing an inducing substrate.

Examples of strong constitutive and/or inducible promoters which are preferred for use in fungal expression hosts are those which are obtainable from the fungal genes for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA gene), acetamidase (amdS) and glyceraldehyde-3-phosphate dehydrogenase (gpd) promoters.

Examples of strong yeast promoters are those obtainable from the genes for alcohol dehydrogenase, lactase, 3-phosphoglycerate kinase and triosephosphate isomerase.

Examples of strong bacterial promoters are the α-amylase and SP02 promoters as well as promoters from extracellular protease genes.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the nucleotide sequence of the present invention. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat Gene 217 [1987] 217-225; and Dawson Plant Mol. Biol. 23 [1993] 97).

Secretion

Often, it is desirable for a polypeptide for use in the present invention to be secreted from the expression host into the culture medium from where the polypeptide of the present invention may be more easily recovered. According to the present invention, the secretion leader sequence may be selected on the basis of the desired expression host. Hybrid signal sequences may also be used with the context of the present invention.

Typical examples of heterologous secretion leader sequences are those originating from the fungal amyloglucosidase (AG) gene (glaA—both 18 and 24 amino acid versions e.g. from Aspergillus), the a-factor gene (yeasts e.g. Saccharomyces and Kluyveromyces) or the α-amylase gene (Bacillus).

Constructs

The term “construct”—which is synonymous with terms such as “conjugate”, “cassette” and “hybrid”—includes a nucleotide sequence for use according to the present invention directly or indirectly attached to a promoter. An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment In some cases, the terms do not cover the natural combination of the nucleotide sequence coding for the protein ordinarily associated with the wild type gene promoter and when they are both in their natural environment.

The construct may even contain or express a marker which allows for the selection of the genetic construct in, for example, a bacterium, preferably of the genus Bacillus, such as Bacillus subtilis, or plants into which it has been transferred. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

For some applications, preferably the construct of the present invention comprises at least the nucleotide sequence of the present invention operably linked to a promoter.

Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an E. coli plasmid to a bacterium, such as of the genus Bacillus, then the transformation vector is sometimes called a “shuttle vector”. It may even be a construct capable of being transferred from an E. coli plasmid to an Agrobacterium to a plant.

The vectors of the present invention may be transformed into a suitable host cell as described below to provide for expression of a polypeptide of the present invention. Thus, in a further aspect the invention provides a process for preparing polypeptides for use according to the present invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter.

The vectors of the present invention may contain one or more selectable marker genes. The most suitable selection systems for industrial micro-organisms are those formed by the group of selection markers which do not require a mutation in the host organism. Examples of fungal selection markers are the genes for acetamidase (amdS), ATP synthetase, subunit 9 (oliC), orotidine-5′-phosphate-decarboxylase (pvrA), phleomycin and benomyl resistance (benA). Examples of non-fungal selection markers are the bacterial G418 resistance gene (this may also be used in yeast, but not in filamentous fungi), the ampicillin resistance gene (E. coli), the neomycin resistance gene (Bacillus) and the E. coli uidA gene, coding for β-glucuronidase (GUS).

Vectors may be used in vitro, for example for the production of RNA or used to transfect or transform a host cell.

Thus, polynucleotides for use according to the present invention can be incorporated into a recombinant vector (typically a replicable vector), for example a cloning or expression vector. The vector may be used to replicate the nucleic acid in a compatible host cell. Thus in a further embodiment, the invention provides a method of making polynucleotides of the present invention by introducing a polynucleotide of the present invention into a replicable vector, introducing the vector into a compatible host cell, and growing the host cell under conditions which bring about replication of the vector. The vector may be recovered from the host cell. Suitable host cells are described below in connection with expression vectors.

The present invention also relates to the use of genetically engineered host cells expressing an amino acid sequence (or variant, homologue, fragment or derivative thereof) according to the present invention in screening methods for the identification of inhibitors and antagonists of said amino acid sequence. Such genetically engineered host cells could be used to screen peptide libraries or organic molecules. Antagonists and inhibitors of said amino acid sequence, such as antibodies, peptides or small organic molecules will provide the basis for pharmaceutical compositions for the treatment of damaged tissue, such as wounds. Such inhibitors or antagonists can be administered alone or in combination with other therapeutics for the treatment of such diseases.

The present invention also relates to expression vectors and host cells comprising a polynucleotide sequences encoding said amino acid sequence, or variant, homologue, fragment or derivative thereof for to screen for agents that can inhibit or antagonise said amino acid sequence.

Expression Vectors

The nucleotide sequence for use in the present invention can be incorporated into a recombinant replicable vector. The vector may be used to replicate and express the nucleotide sequence in and/or from a compatible host cell. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The protein produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

Fusion Proteins

The amino acid sequence of the present invention may be produced as a fusion protein, for example to aid in extraction and purification. Examples of fusion protein partners include glutathione-S-transferase (GST), 6×His, GAL4 (DNA binding and/or transcriptional activation domains) and (β-galactosidase. It may also be convenient to include a proteolytic cleavage site between the fusion protein partner and the protein sequence of interest to allow removal of fusion protein sequences. Preferably the fusion protein will not hinder the activity of the protein sequence.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

Growth Factor

An essential component of the composition of the present invention is the presence and/or use of one or more growth factor(s). The growth factor may be an endogeneous growth factor and/or an exogeneously applied growth factor, which exogeneously applied growth factor may be the same as or similar to an endogeneous growth factor.

In accordance with the present invention, the growth factor may be one or more growth factor(s) that is(are) capable of being efficacious in enhancing damaged tissue, such as wound, healing.

As used herein, the term “growth factor” means a substance (typically a peptidic or proteinacious substance) which stimulates the growth and/or migration of cells that are involved in the damaged tissue, such as wound, healing process, including fibroblasts, keratinocytes and/or endothelial cells. Such a substance may be (or be homologous to or derived from) a protein or peptide produced by cells within the body, in which case it is an endogenous growth factor. In the alternative, it may be have been discovered from libraries of peptidic or proteinacious substances foreign to the human body.

By way of background information, growth factors are discussed in Molecular Biology of The Cell (2^{nd} ed., 1989; Alberts, B., Bray, D., Lewis, J., Raff, M., Roberts, K. & Watson, J. D., eds.), wherein it is stated:

“The conditions that must be satisfied before a cell will grow and divide are considerably more complex for an animal cell than for yeast. If vertebrate cells in a standard artificial culture medium are completely deprived of serum, they normally will not pass the restriction point, even though all the obvious nutrients are present; and they will halt their growth as well as their progress through the chromosome cycle. Painstaking analyses have revealed that the essential components of serum are highly specific proteins, mostly present in very low concentrations (in the order of 10⁻⁹ to 10⁻¹¹ M). Different types of cells require different sets of these proteins. Some of these proteins in serum are directly and specifically involved in stimulating cell division and are called growth factors. One example is platelet-derived growth factor, or PDGF.”

Growth factors are also discussed in WO-A-99/59614.

In cell biology experiments, many growth factors enhance the proliferation and/or motility of the major cell types involved in dermal wound healing, principally keratinocytes and dermal fibroblasts (Singer, A. J. & Clark, R. A. F. (1999) New Engl. J. Med. 341, 738-746). Pharmaceutical preparations of many growth factors have been examined for their efficacy in chronic dermal ulcers. For example, platelet derived growth factor (PDGF), fibroblast growth factor (FGF), transforming growth factor β3 (TGFβ3), keratinocyte-derived growth factor-2 (KGF-2), epidermal growth factor (EGF) and granulocyte macrophage colony stimulating factor (GM-CSF) have all been taken to the clinic to evaluate their efficacy as wound healing agents for chronic dermal ulceration. Whilst these agents have given some encouraging results in animal models of wound healing, only recombinant PDGF (Regranex) has so far demonstrated sufficient efficacy in the clinic to justify its use in the therapy of chronic dermal ulceration.

The reason for the failure of these growth factors to provide pronounced clinical efficacy has been open to much speculation. For example, it has been suggested that the complexity of the wound healing system, involving multiple interacting cell types, and growth factors having actions at distinct temporal phases during the wound healing process, explains why growth factor therapy has not revolutionised wound healing therapy (Borel, J. P. & Maquart, F. X. (1998) Ann. Biol. Clin. (Paris) 56, 11-19). In addition, the half life of growth factors in the wound environment is known to be short, limiting the time available for pharmacological effect. For example, the half life of TGFβ3 after injection into venous ulcers was reported to be approximately 30 minutes.

One hypothesis which explains the short half life of growth factors in chronic dermal ulcers, and their limited clinical efficacy, is that chronic dermal ulcers represent a protease rich environment and that these proteases degrade both growth factors and/or their receptors.

Many proteases have been shown to be over-expressed and/or over-activated in chronic dermal ulcers compared to normal, acute healing wounds. For example, using a variety of biochemical and histological techniques (such as fluid phase protease assays, immunohistochemistry, gel and in situ zymography and ELISAs) matrix metalloproteinases (MMPs), including MMP-13 and MMP-3 (Saarialho-Kere U.K. (1998) Arch. Dermatol. Res. 290, S47-54), neutrophil elastase (Herrick, S., Ashcroft, G., Ireland, G., Horan, M., McCollum, C. & Ferguson, M. (1998) Lab. Invest. 77, 281-8), uPA (Rogers, A. A., Burnett, S., Lindholm, C., Bjellerup, M., Christensen, O. B., Zederfeldt, B., Peschen, M. & Chen, W. Y. (1999) Vasa 28, 101-5) and plasmin (Palolahti, M, Lauharanta, J, Stephens, R W, Kuusela, P, Vaheri. (1993) Exp. Dermatol.2, 29-37), have all been shown to be present in high quantities in either wound fluid from chronic dermal ulcers, or in sections of wound tissue from the same. In addition, it has been shown that when growth factors are added to wound fluid from chronic dermal ulcers, they are proteolytically degraded in vitro (Lauer, G., Flamme, 1., Kreig, T., Sollberg, S. & Eming, S. (1998) J. Invest. Dermatol. 110, 528, abstract 338), and when wound fluid is added to cells in culture, they lose their responsiveness to growth factors.

It is also to be noted that up until now no one had identified which protease(s) is/are responsible for this degradation. This was largely attributable to the fact that up until now accurate modelling of the effects of protease inhibitors on growth factors and their receptors had been impossible to perform. In this regard, many proteases—which are from divergent structural and mechanistic classes and which are over-expressed and over-active in chronic dermal ulceration—activate one another via a network of interacting and circular pathways. Also, some proteases are essential for cell migration and collagen deposition, critical components of normal wound healing, which indicates that unless appropriate selectivity is achieved in protease inhibitors, wound healing would be expected to be impaired (Pilcher, B. K., Wang, M., Qin, X. J., Parks, W. C., Senior, R. M., Welgus, H. G. (1999) Ann. N.Y. Acad. Sci. 878, 12-24). In addition, the level of endogenous inhibitors of proteases (such as Tissue Inhibitors of Metalloproteinases [TIMPs] and plasminogen activator inhibitors [PAIs]) is also altered in chronic dermal ulcers, which adds to the complexity and unpredictability of the pathology (Itoh, Y. & Nagase, H. (1995) J. Biol. Chem. 270, 16518-16521; Knauper, V., Lopez-Otin, C., Smith, B., Knight, G. & Murphy, G. (1996) J. Biol. Chem. 271, 1544-1550). Hence, overall, the effects of specific inhibition of particular proteases on growth factor preservation and function in chronic dermal ulceration up until now were unknown.

In accordance with the present invention, we believe that limiting specific proteolytic degradation affects the efficacy of a variety of growth factors (both endogenous and therapeutically applied) in chronic dermal ulcers. The composition of the present invention therefore concerns specific protease inhibitors, which are used in combination with one or more growth factors. The composition of the present invention overcomes the problem(s) associated with the prior art therapies.

If the inhibitor agent is a protein, then it may be applied topically or orally or intraveneously as that protein (in any formulation). In addition, or in the alterative, the DNA encoding that protein may be applied to the damaged tissue, such as a wound, such as when incorporated into a suitable vector, such as by using a device, such as by way of example a gene gun (e.g. Lu, B., Scott, G. & Goldsmith, L. A. (1996) Proc. Assoc. Am. Physicians 108, 168-172).

The growth factor of the present invention may be applied topically as a protein (in any formulation). In addition, or in the alternative, the DNA encoding the growth factor may be applied to the damaged tissue, such as a wound, such as when incorporated into a suitable vector, such as by using a device, such as by way of example a gene gun (e.g. Lu, B., Scott, G. & Goldsmith, L. A. (1996) Proc. Assoc. Am. Physicians 108, 168-172).

Examples of growth factors for use in the present invention include one or more of PDGF, FGF, CTGF (in particular CTGF-like), KGF (in particular KGF-2), TGF (in particular TGF-β), CSF (in particular GM-CSF), VEGF, EGF, Chrysalin. Details on these growth factors are presented below.

VEGF

A growth factor for use in the composition of the present invention may be VEGF.

Background teachings on this growth factor have been presented by Victor A. McKusick et al on http://www.ncbi.nlm.nih.gov/Omim. For ease of reference, the following information has been extracted from that source.

Many polypeptide mitogens such as basic fibroblast growth factor and platelet-derived growth factors are active on a wide range of different cell types. In contrast, vascular endothelial growth factor is a mitogen primarily for vascular endothelial cells. It is, however, structurally related to platelet-derived growth factor. Tischer et al. (1991) demonstrated that VEGF, also called vascular permeability factor (VPF), is produced by cultured vascular smooth muscle cells. By analysis of transcripts from these cells by PCR and cDNA cloning, they demonstrated 3 different forms of the VEGF coding region. These cDNAs had predicted products of 189, 165, and 121 amino acids. They found that the VEGF gene is split among 8 exons and that the various VEGF coding region forms arise through alternative splicing: the 165-amino acid form is missing the residues encoded by exon 6, whereas the 121-amino acid form is missing the residues encoded by exons 6 and 7. VEGF, a homodimeric glycoprotein of relative molecular mass 45,000, is the only mitogen that specifically acts on endothelial cells. It may be a major regulator of tumor angiogenesis in vivo. Millauer et al. (1994) observed in mouse that its expression was upregulated by hypoxia and its cell-surface receptor, Flk1 is exclusively expressed in endothelial cells. Folkman (1995) noted the importance of VEGF and its receptor system in tumor growth and suggested that intervention in this system may provide promising approaches to cancer therapy. VEGF and placental growth factor constitute a family of regulatory peptides capable of controlling blood vessel formation and permeability by interacting with 2 endothelial tyrosine kinase receptors, FLT1 and KDR(FLK1. See also VEGFB. A third member of this family may be the ligand of the related FLT4 receptor involved in lymphatic vessel development. Soker et al. (1998) described the purification and the expression cloning from tumor cells of a VEGF receptor that binds VEGF165 but not VEGF121. This isoform-specific VEGF receptor (VEGF165R) is identical to human neuropilin-1 a receptor for the collapsin/semaphorin family that mediates neuronal cell guidance. When coexpressed in cells with KDR, neuropilin-1 enhances the binding of VEGF165 to KDR and VEGF165-mediated chemotaxis. Conversely, inhibition of VEGF165 binding to neuropilin-1 inhibits its binding to KDR and its mitogenic activity for endothelial cells. Soker et al. (1998) proposed that neuropilin-1 is a VEGF receptor that modulates VEGF binding to KDR and subsequent bioactivity and therefore may regulate VEGF-induced angiogenesis.

Mattei et al. (1996) used radioactive in situ hybridization to map VEGF to 6p21-p12. Wei et al. (1996) reported the localization of the VEGF gene to chromosome 6p12 by fluorescence in situ hybridization. To explore the possibility that VEGF and angiopoietins collaborate during tumor angiogenesis, Holash et al. (1999) analyzed several different murine and human tumor models. Holash et al. (1999) noted that angiopoietin-1 was antiapoptotic for cultured endothelial cells and expression of its antagonist angiopoietin-2 was induced in the endothelium of co-opted tumor vessels before their regression. In contrast, marked induction of VEGF expression occurred much later in tumor progression, in the hypoxic periphery of tumor cells surrounding the few remaining internal vessels, as well as adjacent to the robust plexus of vessels at the tumor margin. Expression of Ang2 in the few surviving internal vessels and in the angiogenic vessels at the tumor margin suggested that the destabilizing action of angiopoietin-2 facilitates the angiogenic action of VEGF at the tumor rim. Holash et al. (1999) implanted rat RBA mammary adenocarcinoma cells into rat brains. Tumor cells rapidly associated with and migrated along cerebral blood vessels. There was minimal upregulation of VEGF. Holash et al. (1999) suggested that a subset of tumors rapidly co-opts existing host vessels to form an initially well vascularized tumor mass. Perhaps as part of a host defense mechanism there is widespread regression of these initially co-opted vessels, leading to a secondarily avascular tumor and a massive tumor cell loss. However, the remaining tumor is ultimately rescued by robust angiogenesis at the tumor margin.

Carmellet et al. (1996) and Ferrara et al. (1996) observed the effects of targeted disruption of the Vegf gene in mice. They found that formation of blood vessels was abnormal but not abolished in heterozygous VEGF-deficient embryos and even more impaired in homozygous VEGF-deficient embryos, resulting in death at mid-gestation. Similar phenotypes were observed in F(1) heterozygous embryos generated by germline transmission. They interpreted their results as indicating a tight dose-dependent regulation of embryonic vessel development by VEGF. Mice homozygous for mutations that inactivate either of the 2 VEGF receptors also die in utero. However, 1 or more ligands other than VEGF might activate such receptors. Ferrara et al. likewise reported the unexpected finding that loss of a single VEGF allele is lethal in a mouse embryo between days 11 and 12. Angiogenesis and blood-island formation were impaired, resulting in several developmental anomalies. Furthermore, VEGF-null embryonic stem cells exhibited a dramatically reduced ability to form tumors in nude mice.

Springer et al. (1998) investigated the effects of long-term stable production of the VEGF protein by myoblast-mediated gene transfer. Myoblasts were transduced with a retrovirus carrying a murine VEGF164 cDNA and injected into mouse leg muscles. Continuous VEGF delivery resulted in hemangiomas containing localized networks of vascular channels. Springer et al. (1998) demonstrated that myoblast-mediated VEGF gene delivery can lead to complex tissues of multiple cell types in normal adults. Exogenous VEGF gene expression at high levels or of long duration can also have deleterious effects. A physiologic response to VEGF was observed in nonischemic muscle; the response in the adult did not appear to occur via angiogenesis and may have involved a mechanism related to vasculogenesis, or de novo vessel development. Springer et al. (1998) proposed that VEGF may have different effects at different concentrations: angiogenesis or vasculogenesis.

Fukumura et al. (1998) established a line of transgenic mice expressing the green fluorescent protein (GFP) under the control of the promoter for VEGF. Mice bearing the transgene showed green cellular fluorescence around the healing margins and throughout the granulation tissue of superficial ulcerative wounds. Implantation of solid tumors in the transgenic mice led to an accumulation of green fluorescence resulting from tumor induction of host VEGF promoter activity. With time, the fluorescent cells invaded the tumor and could be seen throughout the tumor mass. Spontaneous mammary tumors induced by oncogene expression in the VEGF-GFP mouse showed strong stromal, but not tumor, expression of GFP. In both wound and tumor models, the predominant GFP-positive cells were fibroblasts.

To determine the role of VEGF in endochondral bone formation, Gerber et al. (1999) inactivated VEGF through the systemic administration of a soluble receptor chimeric protein in 24-day-old mice. Blood vessel invasion was almost completely suppressed, concomitant with impaired trabecular bone formation and expansion of the hypertrophic chondrocyte zone. Recruitment and/or differentiation of chondroclasts, which express gelatinase B/matrix metalloproteinase-9, and resorption of terminal chondrocytes decreased. Although proliferation, differentiation, and maturation of chondrocytes were apparently normal, resorption was inhibited. Cessation of the anti-VEGF treatment was followed by capillary invasion, restoration of bone growth, resorption of the hypertrophic cartilage, and normalization of the growth plate architecture. These findings indicated to Gerber et al. (1999) that VEGF-mediated capillary invasion is an essential signal that regulates growth plate morphogenesis and triggers cartilage remodeling. Gerber et al. (1999) concluded that VEGF is an essential coordinator of chondrocyte death, chondroclast function, extracellular matrix remodeling, angiogenesis, and bone formation in the growth plate.

An appropriate amino acid sequence and an appropriate nucleotide sequence are presented in a later section herein.

EGF

A growth factor for use in the composition of the present invention may be EGF.

Background teachings on this growth factor have been presented by Victor A. McKusick et al on http://www.ncbi.nlm.nih.gov/Omim. For ease of reference, the following information has been extracted from that source.

What is now known as epidermal growth factor was first described by Cohen (1962). Epidermal growth factor has a profound effect on the differentiation of specific cells in vivo and is a potent mitogenic factor for a variety of cultured cells of both ectodermal and mesodermal origin (Carpenter and Cohen, 1979). Gray et al. (1983) presented the sequence of a mouse EGF cDNA clone, which suggested that EGF is synthesized as a large protein precursor of 1,168 amino acids. Mature EGF is a single-chain polypeptide consisting of 53 amino acids and having a molecular-mass of about 6,000. Urdea et al. (1983) synthesized the gene for human EGF Smith et al. (1982) synthesized and cloned the gene for human β-urogastrone. Urogastrone is a polypeptide hormone found predominantly in the duodenum and in the salivary glands. It is a potent inhibitor of gastric acid secretion and also promotes epithelial cell proliferation. β-urogastrone contains a single polypeptide chain of 53 amino acids, while gamma-urogastrone has the same sequence of amino acids 1-52 but lacks the carboxyterminal arginine of the β form. Sequence comparison indicates that urogastrone is identical to EGF.

EGF is produced in abundance by the mouse submandibular gland. Tsutsumi et al. (1986) found that sialoadenectomy decreased circulating EGF to levels below detection but did not affect testosterone or FSH levels. At the same time a decrease in spermatids in the testis and mature sperm in the epididymis decreased. The changes were corrected by administration of EGF. A role of EGF in some cases of human male infertility, particularly those with unexplained oligospermia, was proposed. During the immediate-early response of mammalian cells to mitogens, histone H3 is rapidly and transiently phosphorylated by one or more kinases. Sassone-Corsi et al. (1999) demonstrated that EGF-stimulated phosphorylation of H3 requires RSK2, a member of the pp90(RSK) family of kinases implicated in growth control. By the study of human-rodent somatic cell hybrids with a genomic DNA probe, Brissenden et al. (1984) mapped the EGF locus to 4q21-4qter, possibly near TCGF, the locus coding for T-cell growth factor.

Both nerve growth factor and epidermal growth factor are on mouse chromosome 3 but they are on different chromosomes in man: 1p and 4, respectively (Zabel et al., 1985). Zabel et al. (1985) pointed out that mouse chromosome 3 has one segment with rather extensive homology to distal 1p of man and a second with homology to proximal 1p of man. By in situ hybridization, Morton et al. (1986) assigned EGF to 4q25-q27. The receptor for EGF is on chromosome 7.

An appropriate amino acid sequence and an appropriate nucleotide sequence are presented in a later section herein.

PDGF

A growth factor for use in the composition of the present invention may be PDGF.

Teachings on PDGF may be found in WO-A-09713857, WO-A-09108761, WO-A-0931671, U.S. Pat. No. 05034375 and WO-A-09201716.

An appropriate amino acid sequence and an appropriate nucleotide sequence for PDGF A-chain are presented in a later section herein.

An appropriate amino acid sequence and an appropriate nucleotide sequence for PDGF B-chain are presented in a later section herein.

FGF

A growth factor for use in the composition of the present invention may be FGF.

Background teachings on this growth factor are presented by Galzie, Z., Kinsella, A. R. & Smith, J. A. (1997) Fibroblast growth factors and their receptors, Biochem. Cell Biol. 75, 669-685. Another review is by Werner, S. (1998) Cytokine & Growth Factor Reviews 9, 153-165.

An appropriate amino acid sequence and an appropriate nucleotide sequence are presented in a later section herein.

CTGF

A growth factor for use in the composition of the present invention may be CTGF.

Background teachings on this growth factor have been presented by Victor A. McKusick et al on http://www.ncbi.nlm.nih.gov/Omim. For ease of reference, the following information has been extracted from that source.

“Bradham et al. (1991) described a new mitogen produced by human umbilical vein endothelial cells, which they termed connective tissue growth factor. The protein, related to platelet-derived growth factor, was predicted from its cDNA to be a 349-amino acid, 38-kD cysteine-rich secreted protein. Martinerie et al. (1992) identified a locus sharing homology with the nov protooncogene overexpressed in avian nephroblastoma and corresponding to the CTGF gene. They assigned the CTGF gene to 6q23.1 by a combination of study of mouse/human somatic cell hybrids and fluorescence in situ hybridization. They showed that CTGF is situated proximal to MYB. By analysis of Northern blots, Kim et al. (1997) found that CTGF is expressed as a 2.4-kb mRNA in a broad spectrum of human tissues. Sequence comparison revealed that CTGF belongs to a group known as the immediate-early genes, which are expressed after induction by growth factors or certain oncogenes. The immediate-early genes have significant sequence homology to the insulin-like growth factor-binding proteins (IGFBPs) and contain the conserved N-terminal IGFBP motif (see IGFBP7). CTGF shares 28 to 38% amino acid identity with IGFBPs 1-6. Kim et al. (1997) demonstrated that CTGF specifically bound insulin-like growth factors (IGFs), although with relatively low affinity. They proposed that the immediate-early genes, together with IGFBP7, constitute a subfamily of IGFBP genes whose products bind IGFs with low affinity.”

An appropriate amino acid sequence and an appropriate nucleotide sequence are presented in a later section herein.

CTGF-Like

A growth factor for use in the composition of the present invention may be CTGF-like. This growth factor is sometimes referred to as CT58 and WISP-2. It has the following accession numbers: AF074604, AF083500, AF100780, 076076.

Background teachings on this growth factor have been presented by Victor A. McKusick et al on http://www.ncbi.nlm.nih.gov/Omim. For ease of reference, the following information has been extracted from that source.

Pennica et al. (Pennica, D.; Swanson, T. A.; Welsh, J. W.; Roy, M. A.; Lawrence, D. A.; Lee, J.; Brush, J.; Taneyhill, L. A.; Deuel, B.; Lew, M.; Watanabe, C.; Cohen, R. L.; Melhem, M. F.; Finley, G. G.; Quirke, P.; Goddard, A. D.; Hillan, K. J.; Gurney, A. L.; Botstein, D.; Levine, A. J. : WISP genes are members of the connective tissue growth factor family that are up-regulated in Wnt-1-transformed cells and aberrantly expressed in human colon tumors. Proc. Nat. Acad. Sci. 95: 14717-14722, 1998) cloned and characterized 3 genes downstream in the Wnt signaling pathway that are relevant to malignant transformation: WISP1, WISP2, and WISP3. The WISP2 cDNA encodes a 250-amino acid protein that is 73% identical to the mouse protein. The authors found that WISP2 RNA expression was reduced in 79% of human colon tumors, in contrast to WISP1 and WISP3, which were overexpressed in colon tumors. By use of radiation hybrid mapping panels, Pennica et al. (1998) mapped the WISP2 gene to 20q12-q13.

An appropriate amino acid sequence and an appropriate nucleotide sequence are presented in a later section herein.

KGF

A growth factor for use in the composition of the present invention may be KGF, in particular KGF-2.

Background teachings on this growth factor have been presented by Victor A. McKusick et al on http://www.ncbi.nlm.nih.gov/Omim. For ease of reference, the following information has been extracted from that source.

“Rubin et al. (1989) identified a growth factor specific for epithelial cells in conditioned medium of a human embryonic lung fibroblast cell line. Because of its predominant activity in keratinocytes, it was referred to as keratinocyte growth factor. KGF was found to consist of a single polypeptide chain of about 28 kD. It was a potent mitogen for epithelial cells but lacked mitogenic activity on either fibroblasts or endothelial cells. Microsequencing showed an amino-terminal sequence containing no significant homology to any known protein. The release of this growth factor by human embryonic fibroblasts raised the possibility that KGF may play a role in mesenchymal stimulation of normal epithelial cell proliferation. In an addendum, Rubin et al. (1989) noted that by use of all the nucleotide probes based on the N-terminal sequence reported in their paper, they had isolated clones encoding KGF and had found significant structural homology between KGF and the other 5 known members of the fibroblast growth factor (FGF) family.

Werner et al. (1994) assessed the function of KGF in normal and wounded skin by expression of a dominant-negative KGF receptor (176943) in basal keratinocytes. The skin of transgenic mice was characterized by epidermal atrophy, abnormalities in the hair follicles, and dermal hyperthickening. Upon skin injury, inhibition of KGF receptor signaling reduced the proliferation rate of epidermal keratinocytes at the wound edge, resulting in substantially delayed reepithelialization of the wound. Mattei et al. (1995) used isotopic in situ hybridization to map Fgf7 to region F-G of mouse chromosome 2. By analysis of DNA from human-rodent somatic cell hybrids with an exon 1 probe, Kelley et al. (1992) found that FGF7 is located on human chromosome 15. Mouse chromosome 2 presents a conserved region of synteny with 15q13-q22. Thus, the human mutation may reside at this site. Using the murine Fgf7 probe for in situ hybridization to human metaphase chromosomes, Mattei et al. (1995) found signals on chromosome 15. Kelley et al. (1992) found a portion of the KGF gene (comprised of exons 2 and 3, the intron between them, and a 3-prime noncoding segment) that was amplified to approximately 16 copies in the human genome and distributed to multiple chromosomes. Using a cosmid probe encoding KGF exon 1 for fluorescence in situ hybridization, Zimonjic et al. (1997) assigned the KGF7 gene to 15q15-q21.1. In addition, copies of KGF-like sequences hybridizing only with a cosmid probe encoding exons 2 and 3 were localized to dispersed sites on chromosome 2q21, 9p11, 9q12-q13, 18p11, 18q11, 21q11, and 21q21.1. The distribution of KGF-like sequences suggested a role for alphoid DNA in their amplification and dispersion. In chimpanzee, KGF-like sequences were observed at 5 chromosomal sites, which were each homologous to sites in human, while in gorilla a subset of 4 of these homologous sites was identified. In orangutan 2 sites were identified, while gibbon exhibited only a single site. The chromosomal localization of KGF sequences in human and great ape genomes indicated that amplification and dispersion occurred in multiple discrete steps, with initial KGF gene duplication and dispersion occurring in multiple discrete steps, with initial KGF gene duplication and dispersion taking place in gibbon and involving loci corresponding to human chromosomes 15 and 21. The findings of Zimonjic et al. (1997) supported the concept of a closer evolutionary relationship of human with chimpanzee and with primates and a possible selective pressure for KGF dispersion during the evolution of higher primates.”

An appropriate amino acid sequence and an appropriate nucleotide sequence are presented in a later section herein.

TGF

A growth factor for use in the composition of the present invention may be TGF, in particular TGF-β.

Background teachings on this growth factor have been presented by Victor A. McKusick et al on http://www.ncbi.nlm.nih.gov/Omim. For ease of reference, the following information has been extracted from that source.

“TGFβ is a multifunctional peptide that controls proliferation, differentiation, and other functions in many cell types. It was first identified by its ability to cause phenotypic transformation of rat fibroblasts. TGFβ is chemically distinct from TGFα. It has essentially no sequence homology with TGFα or with epidermal growth factor, of which TGFα is an analog. Members of the same gene family as TGFβ include inhibin, which inhibits pituitary secretion of follicle stimulating hormone, and Mullerian inhibitory substance, which is produced by the testis and is responsible for regression of the Mullerian ducts (anlagen of the female reproductive system) in the male embryo. Many cells synthesize TGFβ and almost all of them have specific receptors for this peptide. α and β TGFs are classes of transforming growth factors. TGFβ acts synergistically with TGFα in inducing transformation. It also acts as a negative autocrine growth factor. By somatic cell hybridization and in situ hybridization, Fujii et al. (1985, 1986) assigned TGFβ to 19q13.1-q13.3 in man and to chromosome 7 in the mouse. Dickinson et al. (1990) mapped the Tgfβ-1 gene to mouse chromosome 7. Marquardt et al. (1987) determined the complete amino acid sequence. Dickinson et al. (1990) pointed out that high levels of TGFβ1 mRNA and/or protein have been localized in developing cartilage, endochondral and membrane bone, and skin, suggesting a role in the growth and differentiation of these tissues.

Heldin et at. (1997) discussed new developments in the understanding of the mechanisms used by members of the TGF-β family to elicit their effects on target cells. SMAD proteins mediate TGFβ signaling to regulate cell growth and differentiation. Stroschein et al. (1999) proposed a model of regulation of TGFβ signaling by SnoN in which SnoN maintains the repressed state of TGFβ target genes in the absence of ligand and participates in the negative feedback regulation of TGFβ signaling. To initiate a negative feedback mechanism that permits a precise and timely regulation of TGFβ signaling, TGFβ also induces an increased expression of SnoN at a later stage, which in turn binds to SMAD heteromeric complexes and shuts off TGFβ signaling. Using quantitative PCR in 15 cases of Duchenne muscular dystrophy (DMD) and 13 cases of Becker muscular dystrophy (BMD, as well as 11 spinal muscular atrophy patients (SMA) and 16 controls, Bernasconi et al. (1995) found that TGFβ1 expression as measured by mRNA was greater in DMD and BMD patients than in controls. Fibrosis was significantly more prominent in DMD than in BMD, SMA, or controls. The proportion of connective tissue biopsies increased progressively with age in DMD patients, while TGFβ1 levels peaked at 2 and 6 years of age. Bernasconi et al. (1995) concluded that expression of TGFβ1 in the early stages of DMD may be critical in initiating muscle fibrosis, and suggested that antifibrosis treatment might slow progression of the disease, increasing the utility of gene therapy. Although transforming growth factor-β plays a central role in tissue repair, this cytokine is, as pointed out by Border and Noble (1995), a double-edged sword with both therapeutic and pathologic potential. TGF-β has been implicated also in the pathogenesis of adult respiratory distress syndrome (Shenkar et al., 1994), and the kidney seems to be particularly sensitive to TGF-β-induced fibrogenesis. TGF-β has been implicated as a cause of fibrosis in most forms of experimental and human kidney disease (Border and Noble, 1994).

TGF-β plays an important role in wound healing. A number of pathologic conditions, such as idiopathic pulmonary fibrosis, scleroderma, and keloids, which share the characteristic of fibrosis, are associated with increased TGF-β-1 expression. To evaluate the role of TGF-β-1 in the pathogenesis of fibrosis, Clouthier et al. (1997) used a transgenic approach. They targeted the expression of a constitutively active TGF-β-1 molecule to liver, kidney, and white and brown adipose tissue using the regulatory sequences of the rat phosphoenolpyruvate carboxykinase gene. In multiple lines, targeted expression of the transgene caused severe fibrotic disease. Fibrosis of the liver occurred with varying degrees in severity depending upon the level of expression of the TGFβ1 gene. Overexpression of the transgene in kidney also resulted in fibrosis and glomerular disease, eventually leading to complete loss of renal function. Severe obstructive uropathy (hydronephrosis) was also observed in a number of animals. Expression in adipose tissue resulted in a dramatic reduction in total body white adipose tissue and a marked, though less severe, reduction in brown adipose tissue, producing a lipodystrophy-like syndrome. Introduction of the transgene into the ob/ob background suppressed the obesity characteristic of this mutation; however, transgenic mutant mice developed severe hepato- and splenomegaly. Clouthier et al. (1997) noted that the family of rare conditions known collectively as the lipodystrophies are accompanied in almost all forms by other abnormalities, including fatty liver and cardiomegaly. Metabolic and endocrine abnormalities include either mild or severe insulin resistance, hypertriglyceridemia, and a hypermetabolic state. In a study of 170 pairs of female twins (average age 57.7 years), Grainger et al. (1999) showed that the concentration of active plus acid-activatable latent TGFβ1 is predominantly under genetic control (heritability estimate 0.54). SSCP mapping of the TGFβ1 gene promoter identified 2 single-base substitution polymorphisms. The 2 polymorphisms (G to A at position −800 bp and C to T at position −509 bp) are in linkage disequilibrium. The −509C-T polymorphism was significantly associated with plasma concentration of active plus acid-activatable latent TGFβ1, which explained 8.2% of the additive genetic variance in the concentration. Grainger et al. (1999) suggested, therefore, that predisposition to atherosclerosis, bone diseases, or various forms of cancer may be correlated with the presence of particular alleles at the TGFβ1 locus.

Crawford et al. (1998) showed that thrombospondin-1 is responsible for a significant proportion of the activation of TGFβ1 in vivo. Histologic abnormalities in young TGFβ1 null and thrombospondin-1 null mice were strikingly similar in 9 organ systems. Lung and pancreas pathologies similar to those observed in TGFβ1 null animals could be induced in wildtype pups by systemic treatment with a peptide that blocked the activation of TGFβ1 by thrombospondin-1. Although these organs produced little active TGFβ1 in thrombospondin-1 null mice, when pups were treated with a peptide derived from thrombospondin-1 that could activate TGFβ1, active cytokine was detected in situ, and the lung and pancreatic abnormalities reverted toward wildtype.

Dubois et al. (1995) demonstrated in vitro that pro-TGFβ1 was cleaved by furin to produce a biologically active TGFβ1 protein. Expression of proTGFβ1 in furin-deficient cells produced no TGFβ1, while coexpression of pro-TGFβ1 and furin led to processing of the precursor. Blanchette et al. (1997) showed that furin mRNA levels were increased in rat synovial cells by the addition of TGFβ1. This effect was eliminated by pretreatment with actinomycin-D, suggesting to them that regulation was at the gene transcription level. Treatment of rat synoviocytes and kidney fibroblasts with TGFβ1 or TGFβ2 resulted in increased pro-TGF1 processing, as evidenced by the appearance of a 40-kD immunoreactive band corresponding to the TGFβ1 amino-terminal pro-region. Treatment of these cells with TGFβ2 resulted in a significant increase in extracellular mature TGFβ1. Blanchette et al. (1997) concluded that TGFβ1 upregulates gene expression of its own converting enzyme.”

An appropriate amino acid sequence and an appropriate nucleotide sequence are presented in a later section herein.

CSF

A growth factor for use in the composition of the present invention may be CSF, in particular GM-CSF.

Background teachings on this growth factor have been presented by Victor A. McKusick et al on http://www.ncbi.nlm.nih.gov/Omim. For ease of reference, the following information has been extracted from that source.

“Colony-stimulating factors (CSFs) are proteins necessary for the survival, proliferation, and differentiation of hematopoietic progenitor cells. They are named by the cells they stimulate. Macrophage CSF is known as CSF. Granulocyte-macrophage CSF (CSF2; also symbolized GMCSF) stimulates both cell types. Multi-CSF is known as interleukin-3 (IL3). The CSF in human urine, active in stimulating granulocyte-macrophage colony formation by murine cells, was the first CSF to be purified to homogeneity. It is a glycoprotein of MW 45,000 and is a homodimer. Wong et al. (1985) isolated cDNA clones for human GMCSF. Huebner et al. (1985) assigned the GMCSF locus to 5q21-q32 by somatic cell hybrid analysis and in situ hybridization. This is the same region as that involved in interstitial deletions in the 5q-syndrome and acute myelogenous leukemia. They found a partially deleted GMCSF allele and a 5q-marker chromosome in a human promyelocytic leukemia cell line. The truncated GMCSF gene appeared to lie at the rejoining point for the interstitial deletion. By in situ hybridization, Le Beau et al. (1986) assigned FMS to 5q33 and GMCSF to 5q23-q31. Both genes were deleted in the 5q-chromosome from bone marrow cells of 2 patients with refractory anemia and del(5)(q15q33.3). From study of other cases they concluded that FMS is located in band 5q33.2 or 5q33.3 rather than 5q34-q35 as reported earlier. Pettenati et al. (1987) concluded that the order of loci from the centromere toward 5qter is CSF2, CSF1, and FMS (164770). By long-range mapping, Yang et al. (1988) demonstrated that the GMCSF and IL3 genes are separated by about 9 kilobases of DNA. They are tandemly arranged head to tail with IL3 on the 5-prime side of GMCSF. Frolova et al. (1991) identified 2 RFLPs in a 70-kb segment of genomic DNA that includes these 2 genes as well as flanking sequences. Using these markers in studies of the panel from the Centre d'Etude du Polymorphisme Humain (CEPH), they studied linkage with a number of other expressed genes on chromosome 5. Thangavelu et al. (1992) presented a physical and genetic linkage map that encompassed 14 expressed genes and several markers located in the distal half of the long arm of chromosome 5. By fluorescence in situ hybridization, Le Beau et al. (1993) mapped the CSF2 gene to 5q31.1.

Group B streptococcus (GBS) is the most common bacterial infection causing pneumonia and sepsis in newborn infants. Host responses to GBS include activation of both alveolar macrophages and polymorphonuclear leukocytes. Phagocytosis and killing of GBS in the lungs is enhanced by surfactant protein A, which increases phagocytosis and reactive oxygen species-mediated killing. Because macrophage function is strongly influenced by GMCFS, LeVine et al. (1999) tested whether GBS clearance from the lungs was influenced by GMCFS in vivo. Mice homozygous for a knockout of the Cfs2 gene cleared group B streptococcus from the lungs more slowly than wildtype mice. Expression of GMCSF in the respiratory epithelium of homozygous deficient mice improved bacterial clearance to levels greater than that in wildtype mice. Acute aerosolization of GMCSF to wildtype mice significantly enhanced clearance of GBS at 24 hours. In the homozygous knockout mice, GBS infection was associated with increased neutrophilic infiltration in lungs, while macrophage infiltrates predominated in wildtype mice, suggesting an abnonmality in macrophage clearance of bacteria in the absence of GMCSF. While phagocytosis of GBS was unaltered, production of superoxide radicals and hydrogen peroxide was markedly deficient in macrophages from homozygous knockout mice.”

An appropriate amino acid sequence and an appropriate nucleotide sequence are presented in a later section herein.

Chrysalin

A growth factor for use in the composition of the present invention may be Chrysalin. Chrysalin is being developed by Chrysalis Biotechnology Inc. Chrysalin is a small (12 residue) peptide derived from the sequence of thrombin. Chrysalin is described in EP-A-0328552.

Tissue Damaged Upregulated Proteins

In accordance with the present invention, use is made of selective inhibitors of adverse proteins (in particular adverse proteases that have a deleterious effect on wound healing) that are upregulated in a damaged tissue, such as a wound, environment.

The damaged tissue environment for treatment may be a chronic wound, such as a chronic dermal ulcer.

In addition, or in the alternative, the damaged tissue environment for treatment may be one or more those associated with age-related macular degeneration, corneal ulceration, corneal melting, irritable bowel syndrome/disorder/disease, gastric ulceration, renal failure, peripheral neuropathies (e.g. diabetic retinopathy), neurodegenerative diseases, bone diseases or injury, cartilage diseases or injury, muscle diseases or injury, tendon diseases or injury, ischaemic damage, peridontal disease, psoriasis, bullous pemphigoid, epidemnolysis bullosa, spinal cord disease or injury.

Preferably said damaged tissue is a wound, more preferably a chronic wound, such as a chronic dermal ulcer.

In particular, use is made of selective inhibitors of proteases that are upregulated in a damaged tissue, such as a wound, environment, in particular a chronic wound environment, such as chronic dermal ulcers. In this respect, the composition of the present invention comprises an agent that targets one or more of said proteins in order to act as an inhibitor against said protein.

In another embodiment, one or more of said proteins are used in an assay to screen for agents that are capable of inhibiting said proteins. The identified agents are then used to prepare a composition according to the present invention.

Examples of protease proteins that are upregulated in a damaged tissue, such as a wound, environment, in particular a chronic wound environment, such as chronic dermal ulcers, are plasminogen activators and certain matrix metalloproteinases. A particular example of a suitable plasminogen activator is urokinase-type plasminogen activator. Particular examples of matrix metalloproteinases are matrix metalloproteinase 1, matrix metalloproteinase 2, matrix metalloproteinase 3, matrix metalloproteinase 7, matrix metalloproteinase 8, matrix metalloproteinase 9, matrix metalloproteinase 10, matrix metalloproteinase 11, matrix metalloproteinase 12, matrix metalloproteinase 13, matrix metalloproteinase 14, matrix metalloproteinase 15, matrix metalloproteinase 16, matrix metalloproteinase 17, matrix metalloproteinase 19, matrix metalloproteinase 20, matrix metalloproteinase 21, matrix metalloproteinase 24, and matrix metalloproteinase FMF. Details on some of these proteins are presented below.

Urokinase

In accordance with the present invention, a target for the inhibitor agent of the present invention—or a putative inhibitor agent in an assay of the present invention—may be urokinase-type plasminogen activator (uPA).

Urokinase (urinary-type plasminogen activator or uPA; International Union of Biochemistry classification number EC.3.4.21.31) is a serine protease produced by a large variety of cell types (smooth muscle cells, fibroblasts, endothelial cells, macrophages and tumour cells). It has been implicated as playing a key role in cellular invasion and tissue remodelling. A principal substrate for uPA is plasminogen which is converted by cell surface-bound uPA to yield the serine protease plasmin. Locally produced high plasmin concentrations mediate cell invasion by breaking down the extracellular matrix. Important processes involving cellular invasion and tissue remodelling include wound repair, bone remodelling, angiogenesis, tumour invasiveness and spread of metastases.

In particular, uPA is one of the proteases which is over-expressed in chronic dermal ulcers. uPA is a serine protease produced by a large variety of cell types (smooth muscle cells, fibroblasts, endothelial cells, macrophages and tumour cells). It has been implicated as playing a key role in cellular invasion and tissue remodelling. A principal substrate for uPA is plasminogen which is converted by cell surface-bound uPA to yield the serine protease plasmin.

Beneficial effects of urokinase inhibitors have been reported using anti-urokinase monoclonal antibodies and certain other known urokinase inhibitors. For instance, anti-urokinase monoclonal antibodies have been reported to block tumour cell invasiveness in vitro (W. Hollas, et al, Cancer Res. 51:3690; A. Meissauer, et al, Exp. Cell Res. 192:453 (1991); tumour metastases and invasion in vivo (L. Ossowski, J. Cell Biol. 107:2437 (1988)); L. Ossowski, et al, Cancer Res. 51:274 (1991)) and angiogenesis in vivo (J. A. Jerdan et al, J. Cell Biol. 115[3 Pt 2]:402a (1991). Also, Amiloride™, a known urokinase inhibitor of only moderate potency, has been reported to inhibit tumour metastasis in vivo (J. A. Kellen et al, Anticancer Res., 8:1373 (1988)) and angiogenesis/capillary network formation in vitro (M. A. Alliegro et al, J. Cell Biol. 115[3 Pt 2]:402a).

Conditions of particular interest for treatment by urokinase inhibitors include chronic dermal ulcers (including venous ulcers, diabetic ulcers and pressure sores), which are a major cause of morbidity in the ageing population and cause a significant economic burden on healthcare systems. Chronic dermal ulcers are characterised by excessive uncontrolled proteolytic degradation resulting in ulcer extension, loss of functional matrix molecules (e.g. fibronectin) and retardation of epithelisation and ulcer healing. A number of groups have investigated the enzymes responsible for the excessive degradation in the wound environment, and the role of plasminogen activators has been highlighted (M. C. Stacey et al., Br. J. Surgery, 80, 596; M. Palolahti et al., Exp. Dermatol., 2, 29, 1993; A. A. Rogers et al., Wound Repair and Regen., 3, 273, 1995). Normal human skin demonstrates low levels of plasminogen activators which are localised to blood vessels and identified as tissue type plasminogen activator (tPA). In marked contrast, chronic ulcers demonstrate high levels of urokinase type plasminogen activator (uPA) localised diffusely throughout the ulcer periphery and the lesion, and readily detectable in wound fluids.

uPA could affect wound healing in several ways. Plasmin, produced by activation of plasminogen, can produce breakdown of extracellular matrix by both indirect (via activation of matrix metalloproteases) and direct means. Plasmin has been shown to degrade several extracellular matrix components, including gelatin, fibronectin, proteoglycan core proteins as well as its major substrate, fibrin. Whilst activation of matrix metalloproteases (MMPs) can be performed by a number of inflammatory cell proteases (e.g. elastase and cathepsin G), the uPA/plasmin cascade has been implicated in the activation of MMPs in situ, providing a broad capacity for degrading all components of the extracellular matrix. Furthermore, and in addition to its effect on production of plasmin, uPA has been shown to catalyse direct cleavage of fibronectin yielding antiproliferative peptides. Thus, over-expression of uPA in the wound environment has the potential to promote uncontrolled matrix degradation and inhibition of tissue repair. Inhibitors of the enzyme thus have the potential to promote healing of chronic wounds.

Further background teachings on uPA have been presented by Victor A. McKusick et al on http://www.ncbi.nlm.nih.gov/Omim. For ease of reference, the following information has been extracted from that source.

“Urokinase is the urinary plasminogen activator. (Tissue plasminogen activator is a second type; it has a single polypeptide chain of 70,000 daltons and is unrelated to urokinase immunologically.) Urokinase is a protein that has a molecular weight of about 54,000 daltons and is composed of 2 disulfide-linked chains, A and B, of molecular weights 18,000 and 33,000, respectively. Salerno et al. (1984) developed separate monoclonal antibodies for the A and B chains and by using them identified a single-chain biosynthetic precursor in a rabbit reticulocyte cell-free protein-synthesizing system directed by human kidney total polyadenylated RNA. Thus, the precursor must be cleaved in a way that the insulin precursor is cleaved.

By combined somatic cell genetics, in situ hybridization, and Southern hybridization, Tripputi et al. (1985) localized the human urokinase gene to 10q24-qter. By use of specific cDNA probes in the study of human-mouse somatic cell hybrids, Rajput et al. (1985) mapped the human plasminogen activator and urokinase genes to chromosomes 8 and 10, respectively. By Southern blot analysis of DNA from mouse-Chinese hamster and mouse-rat somatic cell hybrids, Rajput et al. (1987) assigned the mouse equivalent (Plau) to mouse chromosome 14. Urokinase may occur as a single-chain form or as a 2-chain derivative, which is generated by cleavage of the peptide bond between lys(158) and ile(159) in the single-chain form by plasmin. Lijnen et al. (1988) produced site-specific mutation in position 158 (lys-to-glu). Studies of the enzymatic properties of the mutant form, which was resistant to plasmin, indicated that the amino acid in position 158 is a main determinant of the functional properties of the single-chain form, but not of the 2-chain form.”

An appropriate amino acid sequence and an appropriate nucleotide sequence are presented in a later section herein.

MMP

In accordance with the present invention, a target for the inhibitor agent of the present invention—or a putative inhibitor agent in an assay of the present invention—may be one or more matrix metalloproteinases (MMPs) wherein said MMP has a deleterious effect on wound heating in damaged tissue.

MMPs constitute a family of structurally similar zinc-containing metalloproteases, which are involved in the remodelling, repair and degradation of extracellular matrix proteins, both as part of normal physiological processes and in pathological conditions. At least 18 members of the human family have been sequenced.

Since they have high destructive potential, the MMPs are usually under close regulation, and failure to maintain MMP regulation has been implicated as a component of a number of conditions. Examples of conditions where MMPs are thought to be important are those involving bone restructuring, embryo implantation in the uterus, infiltration of immune cells into inflammatory sites, ovulation, spermatogenesis, tissue remodelling during wound repair and organ differentiation such as such as in venous and diabetic ulcers, pressure sores, colon ulcers for example ulcerative colitis and Crohn's disease, duodenal ulcers, fibrosis, local invasion of tumours into adjacent areas, metastatic spread of tumour cells from primary to secondary sites, and tissue destruction in arthritis, skin disorders such as dystrophic epidermolysis bulosa, dermatitis herpetiformis, or conditions caused by or complicated by embolic phenomena, such as chronic or acute cardiac or cerebral infarctions.

Substrates for the MMPs are diverse—and sometimes include other members of the gene family. For example, MMP-14 is known to digest and activate proMMP-2 and both MMP-3 and MMP-9 can digest and activate proMMP-1. Some MMP substrates are also matrix components—such as collagen which is digested, for example by MMP-1 (also known as collagenase-1), denatured collagen or gelatin which is digested for example, by MMP-2 (also known as gelatinase-A), fibronectin which is digested for example by MMP-3 (allso known as stromelysin-1) and glycosaminoglycans which is digested for example by MMP-3.

For recent reviews of MMPs, see Zask et al, Current Pharmaceutical Design, 1996, 2, 624-661; Beckett, Exp. Opin. Ther. Patents, 1996, 6, 1305-1315; and Beckett et al, Drug Discovery Today, vol 1(no.1), 1996, 16-26.

Alternative names for various MMPs and substrates acted on by these are shown in the table below (Zask et al, supra).

| Enzyme | Other names | Preferred substrates |
|---|---|---|
| MMP-1 | Collagenase-1, interstitial | Collagens I, II, III, VII, X, gelatins |
| | collagenase | |
| MMP-2 | Gelatinase A, 72 kDa | Gelatins, collagens IV, V, VII, X, |
| | gelatinase | elastin, fibronectin; activates pro- |
| | | MMP-13 |
| MMP-3 | Stromelysin-1 | Proteoglycans, laminin, |
| | | fibronectin, gelatins. |
| MMP-7 | Pump, Matrilysin | Proteoglycans, laminin, |
| | | fibronectin, gelatins, collagen IV, |
| | | elastin, activates pro-MMP-1 |
| | | and -2. |
| MMP-8 | Collagenase-2, neutrophil | Collagens I, II, III |
| | collagenase | |
| MMP-9 | Gelatinase B, 92 kDa | Gelatins, collagens IV, V, elastin |
| | gelatinase | |
| MMP-12 | Macrophage | Elastin, collagen IV, fibronectin, |
| | metalloelastase | activates pro-MMP-2 & 3. |
| MMP-13 | Collagenase-3 | Collagens I, II, III, gelatins |
| MMP-14 | MT-MMP-1 | Activates pro-MMP-2 & 13, |
| | | gelatins |
| MMP-15 | MT-MMP-2 | |
| MMP-16 | MT-MMP-3 | Activates pro-MMP-2 |
| MMP-17 | MT-MMP-4 | |

Examples of suitable MMP target(s) for the inhibitor agent of the present invention—or for a putative inhibitor agent in an assay of the present invention—may be any suitable member of one or more of: matrix metalloproteinase I (MMP1), matrix metalloproteinase 2 (MMP2), matrix metalloproteinase 3 (MMP3), matrix metalloproteinase 7 (MMP7), matrix metalloproteinase 8 (MMP8), matrix metalloproteinase 9 (MMP9), matrix metalloproteinase 10 (MMP10), matrix metalloproteinase 11 (MMP11), matrix metalloproteinase 12 (MMP12), matrix metalloproteinase 13 (MMP13), matrix metalloproteinase 14 (MMP14), matrix metalloproteinase 15 (MMP15), matrix metalloproteinase 16 (MMP16), matrix metalloproteinase 17 (MMP17), matrix metalloproteinase 19 (MMP19), matrix metalloproteinase 20 (MMP20), matrix metalloproteinase 21 (MMP21), matrix metalloproteinase 24 (MMP24), and matrix metalloproteinase FMF (MMPFMF).

Some of these targets are discussed in slightly more detail. In addition, appropriate amino acid sequences and appropriate nucleotide sequences are presented in a later section herein.

For some embodiments of the present invention, preferably the target for the inhibitor agent of the present invention may be MMP13 and/or MMP3.

MMP1

For some embodiments of the present invention, the target for the inhibitor agent of the present invention may be MMP1.

Background teachings on matrix metalloproteinase I (MMP1) have been presented by Victor A. McKusick et al on http://www.ncbi.nlm.nih.gov/Omim. For ease of reference, the following information has been extracted from that source.

“Brinckerhoff et al. (1987) identified a cDNA clone of human collagenase (EC 3.4.23.7 ). The clone identified a single collagenase gene of about 17 kb from blots of human genomic DNA. Restriction enzyme analysis and DNA sequence data indicated that the cDNA clone was full length and that it was identical to that described for human skin fibroblast collagenase. Collagenase is the only enzyme able to initiate breakdown of the interstitial collagens, types I, II, and III. The fact that the collagens are the most abundant proteins in the body means that collagenase plays a key role in the remodeling that occurs constantly in both normal and diseased conditions. The identity of human skin and synovial cell collagenase and the ubiquity of this enzyme and of its substrates, collagens I, II, and III, imply that the common mechanism controlling collagenolysis throughout the body may be operative in both normal and disease states. Gerhard et al. (1987) confirmed the assignment of the collagenase gene to chromosome 11 by the use of a DNA probe for Southern analysis of somatic cell hybrids. Analysis of cell lines with rearrangements involving chromosome 11 indicated that the gene is in the region 11q11-q 23. Church et al. (1983) had used somatic cell hybrids between mouse cells and human normal skin and corneal fibroblasts and recessive dystrophic epidermolysis bullosa (RDEB) skin fibroblasts to assign the human structural gene for collagenase to chromosome 11. Production of collagenase was measured by a specific radioimmunoassay. It appeared that both the normal and the RDEB collagenase gene mapped to chromosome 11. This was earlier taken to indicate that the abnormal collagenase produced by RDEB cells represented a mutation of the structural gene. Later work indicated that both the autosomal dominant (131750) and autosomal recessive forms of dystrophic epidermolysis bullosa are due to mutations in the type VII collagen gene (COL7A1; 120120). The excessive formation of collagenase must represent a secondary phenomenon, not the primary defect. It should be noted that fibroblasts from patients with the Werner syndrome also express high constitutive levels of collagenase in vitro (Bauer et al., 1986). Pendas et al. (1996) isolated a 1.5-Mb YAC clone mapping to 11q22. Detailed analysis of this nonchimeric YAC clone ordered 7 MMP genes as follows: cen—MMP8—MMP10—MMP1—MMP3—MMP12—MMP7—MMP13—tel.

Note on nomenclature: In reporting on the nomenclature of the matrix metalloproteinases, Nagase et al. (1992) referred to interstitial collagenase as MMP1.”

MMP2

For some embodiments of the present invention, the target for the inhibitor agent of the present invention may be MMP2.

Background teachings on matrix metalloproteinase 2 (MMP2) have been presented by Victor A. McKusick et at on http://www.ncbi.nlm.nih.gov/Omim. For ease of reference, the following information has been extracted from that source.

“Type IV collagenase is a metalloproteinase that specifically cleaves type IV collagen, the major structural component of basement membranes. The metastatic potential of tumor cells has been found to correlate with the activity of this enzyme. Huhtala et al. (1990) determined that the CLG4A gene is 17 kb long with 13 exons varying in size from 110 to 901 bp and 12 introns ranging from 175 to 4,350 bp. Alignment of introns showed that introns 1 to 4 and 8 to 12 of the type IV collagenase gene coincide with intron locations in the interstitial collagenase and stromelysin genes, indicating a close structural relationship of these metalloproteinase genes. Devarajan et al. (1992) reported on the structure and expression of 78-kD gelatinase, which they referred to as neutrophil gelatinase. Type IV collagenase, 72-kD, is officially designated matrix metalloproteinase-2 (MMP2). It is also known as gelatinase, 72-kD (Nagase et al., 1992). Irwin et al. (1996) presented evidence that MMP2 is a likely effector of endometrial menstrual breakdown. They cultured human endometrial stromal cells in the presence of progesterone and found an augmentation of proteinase production after withdrawal of proteinase: the same results were achieved by the addition of the P receptor antagonist RU486. Characterization of the enzyme by Western blotting revealed it to be MMP2. Northern blot analysis showed differential expression of MMP2 mRNA in late secretory phase endometrium.

Angiogenesis depends on both cell adhesion and proteolytic mechanisms. Matrix metalloproteinase-2 and integrin α-V/β-3 are functionally associated on the surface of angiogenic blood vessels. Brooks et al. (1998) found that a fragment of MMP2, which comprises the C-terminal hemopexin-like domain (amino acids 445-635) and is termed PEX, prevents this enzyme from binding to α-V/β-3 and blocks cell surface collagenolytic activity in melanoma and endothelial cells. PEX blocks MMP2 activity on the chick chorioallantoic membrane where it disrupts angiogenesis and tumor growth. Brooks et al. (1998) also found that a naturally occurring form of PEX can be detected in vivo in conjunction with α-V/β-3 expression in tumors and during developmental retinal neovascularization. Levels of PEX in these vascularized tissues suggest that it interacts with endothelial cell α-V/β-3 where it serves as a natural inhibitor of MMP2 activity, thereby regulating the invasive behavior of new blood vessels. The authors concluded that recombinant PEX may provide a potentially novel therapeutic approach for diseases associated with neovascularization.

By hybridization to a panel of DNAs from human-mouse cell hybrids and by in situ hybridization using a gene probe, Fan et al. (1989) assigned the CLG4 gene to 16q21; see Huhtala et al. (1990). By hybridization to somatic cell hybrid DNAs, Collier et al. (1991) assigned both CLG4A and CLG4B to chromosome 16. Chen et al. (1991) mapped 12 genes on the long arm of chromosome 16 by the use of 14 mouse/human hybrid cell lines and the fragile site FRA16B. The breakpoints in the hybrids, in conjunction with the fragile site, divided the long arm into 14 regions. They concluded that CLG4 is in band 16q13.

Morgunova et al. (1999) reported the crystal structure of the full-length proform of human MMP2. The crystal structure revealed how the propeptide shields the catalytic cleft and that the cysteine switch may operate through cleavage of loops essential for propeptide stability. Becker-Follmann et al. (1997) created a high-resolution map of the linkage group on mouse chromosome 8 that is conserved on human 16q. The map extended from the homolog of the MMP2 locus on 16q13 (the most centromeric locus) to CTRB on 16q23.2-q23.3.”

MMP3

For some embodiments of the present invention, the target for the inhibitor agent of the present invention may be MMP3.

Thus, according to this embodiment, the present invention provides a pharmaceutical for use in damaged tissue, such as wound, treatment (e.g. healing); the pharmaceutical comprising a composition which comprises: (a) a growth factor; and an inhibitor agent; and optionally c) a pharmaceutically acceptable carrier, diluent or excipient; wherein the inhibitor agent can inhibit the action of at least one specific adverse protein (e.g. a specific protease) that is upregulated in a damaged tissue, such as a wound, environment; wherein said specific protein is MMP3.

Background teachings on matrix metalloproteinase 3 (MMP3) have been presented by Victor A. McKusick et al on http://www.ncbi.nlm.nih.gov/Omim. For ease of reference, the following information has been extracted from that source.

“Human fibroblast stromelysin (also called transin or matrix metalloproteinase-3) is a proteoglycanase closely related to collagenase (MMP1) with a wide range of substrate specificities. It is a secreted metalloprotease produced predominantly by connective tissue cells. Together with other metalloproteases, it can synergistically degrade the major components of the extracellular matrix (Sellers and Murphy, 1981). Stromelysin is capable of degrading proteoglycan, fibronectin, laminin, and type IV collagen, but not interstitial type I collagen. Whitham et al. (1986) found that the amino acid sequences predicted from the cDNAs of collagenase and stromelysin indicate that they are closely related enzymes, with a particularly well-conserved region of 14 amino acids, that shares significant homology with the zinc-chelating region of the bacterial metalloprotease thermolysin (Matthews et al., 1974).

Wilhelm et al. (1987) purified and determined the complete primary structure of human stromelysin. It is synthesized in a preproenzyme form with a calculated size of 53,977 Da and a 17-amino acid long signal peptide. A comparison of primary structures suggested that stromelysin is the human analog of rat transin. Saus et al. (1988) determined the complete primary structure of human matrix metalloproteinase-3 (MMP3), which has 477 amino acid residues, including a 17-residue signal peptide. The findings indicated that MMP3 is identical to stromelysin. MMP3 and collagenase were found to be 54% identical in sequence, suggesting a common evolutionary origin of the 2 proteinases.

Furthermore, MMP3 and collagenase expression appeared to be coordinately modulated in synovial fibroblast cultures. Levels of mRNA for both proteins are induced by interleukin-1-β and suppressed by retinoic acid or dexamethasone. Koklitis et al. (1991) purified 2 forms of recombinant human prostromelysin. By somatic cell hybridization and in situ hybridization, Spurr et al. (1988) mapped the stromelysin locus to 11q and confirmed the location of the collagenase gene on chromosome 11, specifically on 11q. Gatti et al. (1989) placed the STMY locus in the 11q22-q23 region by linkage analysis with markers in that area, including ataxia-telangiectasia. By pulsed field gel electrophoresis, Formstone et al. (1993) showed that a cluster of metalloproteinase genes—stromelysin I, fibroblast collagenase (MMP1), and stromelysin II (MMP10)—are located in a 135-kb region of chromosome 11. The physical proximity of these 3 genes, together with the DNA marker D11S385, was confirmed using 2 YAC clones, and their relative order determined. This information, combined with the pattern of marker representation in a panel of radiation-reduced chromosome 11 hybrids, suggested that the order was cen—STMY2—CLG-STMY1—D11S385—ter. Pendas et al. (1996) noted that the family of human MMPs was composed of 14 members at the time of their report. MMP genes have been mapped to chromosomes 11, 14 (MMP14, 16 (MMP2, 20 (MMP9), and 22 (MMP11), with several clustered within the long arm of chromosome 11. Pendas et al. (1996) isolated a 1.5-Mb YAC clone mapping to 11q22. Detailed analysis of this nonchimeric YAC clone ordered 7 MMP genes as follows: cen—MMP8—MMP10—MMP1—MMP3—MMP12—MMP7—MMP13 tel. Kerr et al. (1988) examined the role of FOS (164810) in growth-factor stimulation of transin, a matrix-degrading secreted metalloproteinase. The stimulatory effect of both platelet-derived growth factor (190040) and epidermal growth factor on transin transcription involved factors recognizing the sequence TGAGTCA, which is found in the transin promoter and is a binding site for the transcriptional factor JUN/AP1 and for associated FOS and FOS-related complexes.

Wound repair involves cell migration and tissue remodeling, and these ordered and regulated processes are facilitated by matrix-degrading proteases. Saarialho-Kere et al. (1992) found that interstitial collagenase is invariantly expressed by basal keratinocytes at the migrating front of healing epidermis. Because the substrate specificity of collagenase is limited principally to interstitial fibrillar collagens, other enzymes must also be produced in the wound environment to restructure tissues effectively with a complex matrix composition. The stromelysins can degrade many noncollagenous connective tissue macromolecules. Using in situ hybridization and immunohistochemistry, Saarialho-Kere et al. (1994) found that both stromelysin I and stromelysin II are produced by distinct populations of keratinocytes in a variety of chronic ulcers. Stromelysin I mRNA and protein were detected in basal keratinocytes adjacent to but distal from the wound edge in what probably represented the sites of proliferating epidermis. In contrast, stromelysin II mRNA was seen only in basal keratinocytes at the migrating front, in the same epidermal cell population that expressed collagenase. Stromelysin I producing keratinocytes resided on the basement membrane, whereas stromelysin II producing keratinocytes were in contact with the dermal matrix. Furthermore, stromelysin I expression was prominent in dermal fibroblasts, whereas no signal for stromelysin II was seen in any dermal cell. These findings demonstrated that the 2 stromelysins are produced by different populations of basal keratinocytes in response to wounding and suggested that they serve distinct roles in tissue repair.

Using immunofluorescence staining, RT-PCR, and in situ hybridization, Lu et al. (1999) localized stromelysin I to the epithelial layers of unwounded and wounded corneas. They found stromelysin I in the deep stromal layer in the first 3 days after wounding and in the area of newly synthesized stromal matrix 1 week after surgery. They stated that stromelysin I activates matrilysin (MMP7) (Imai et al., 1995) and that stromelysin I and matrilysin interact during tissue remodeling. They concluded that stromelysin I may be involved in the reparative process in the wound bed after excimer keratectomy, whereas matrilysin may play a role in epithelial wound remodeling not only in the migration phase but also in the subsequent proliferation phase.

There is a common polymorphism in the promoter sequence of the STMY1 gene, with 1 allele containing a run of 6 adenosines (6A) and the other 5 adenosines (5A). Ye et al. (1996) followed up on a previously reported 3-year study by Richardson et al. 1989) of patients with coronary atherosclerosis which indicated that those patients who were homozygous for the 6A allele showed a more rapid progression of both global and focal atherosclerotic stenoses. This observation supported the findings by others that the metalloproteinases are involved in connective tissue remodeling during atherogenesis. Ye et al. (1996) investigated whether the 5A/6A promoter polymorphism plays a role in the regulation of STMY1 gene expression. In transient expression experiments, a STMY1 promoter construct with 6A at the polymorphic site was found to express less of the reporter gene than a construct containing 5A. Binding of a nuclear protein factor was more readily detectable with an oligonucleotide probe corresponding to the 6A allele as compared with a probe corresponding to the 5A allele. Thus, Ye et al. (1996) found that the 5A/6A polymorphism appears to play an important role in regulating STMY1 expression. In a study by Quinones et al. (1989), the frequency of the 2 alleles (5A/6A) was found to be 0.51/0.49 in a sample of 354 healthy individuals from the UK.

Sternlicht et al. (1999) examined how MMP3, or STR1, affects tumor progression using 2 genetic approaches: phenotypically normal mammary epithelial cells that express STR1 in a tetracycline-regulated manner, and an STR1 transgene targeted to mouse mammary glands by the mouse ‘whey acidic protein’ (WAP) gene promoter. Phenotypically normal mammary epithelial cells with tetracycline-regulated expression of STR1 formed epithelial glandular structures in vivo without STR1 but formed invasive mesenchymal-like tumors with STR1. Once initiated, the tumors became independent of continued STR1 expression. STR1 also promoted spontaneous premalignant changes and malignant conversion in mammary glands of transgenic mice. These changes were blocked by coexpression of a TIMP1 (305370) transgene. The premalignant and malignant lesions had stereotyped genomic changes unlike those seen in other murine mammary cancer models. These data indicated that STR1 influences tumor initiation and alters neoplastic risk.”

MMP7

For some embodiments of the present invention, the target for the inhibitor agent of the present invention may be MMP7.

Background teachings on this matrix metalloproteinase have been presented by Victor A. McKusick et al on http://www.ncbi.nlm.nih.gov/Omim. For ease of reference, the following information has been extracted from that source.

The putative metalloproteinase 1 (PUMP1) gene was identified through studies of collagenase-related connective-tissue-degrading metalloproteinases produced by human tumors. Muller et al. (Muller, D.; Quantin, B.; Gesnel, M. C.; Millon-Collard, R.; Abecassis, J.; Breathnach, R.: The collagenase gene family in humans consists of at least four members. Biochem. J. 253: 187-192, 1988) found that the PUMP protein has 267 amino acids and is significantly shorter than stromelysin or collagenase (477 and 469 amino acids, respectively), Putative metalloproteinase 1 was later called matrilysin or matrix metalloproteinase-7 (MMP7).

MMP8

For some embodiments of the present invention, the target for the inhibitor agent of the present invention may be MMP8.

Background teachings on this matrix metalloproteinase have been presented by Victor A. McKusick et al on http://www.ncbi.nlm.nih.gov/Omim. For ease of reference, the following information has been extracted from that source.

Neutrophil collagenase, a member of the family of matrix metalloproteinases, is distinct from the collagenase of skin fibroblasts and synovial cells in substrate specificity and immunologic crossreactivity. Hasty et al. (Hasty, K. A.; Pourmotabbed, T. F.; Goldberg, G. I.; Thompson, J. P.; Spinella, D. G.; Stevens, R. M.; Mainardi, C. L. : Human neutrophil collagenase: a distinct gene product with homology to other matrix metalloproteinases. J. Biol. Chem. 265: 11421-11424, 1990.) cloned and sequenced a cDNA encoding human neutrophil collagenase using a lambda-gt11 cDNA library constructed from mRNA extracted from the peripheral leukocytes of a patient with chronic granulocytic leukemia. The coding sequence predicts a 467-amino acid protein. It hybridized to a 3.3-kb mRNA from human bone marrow. Other features of the primary structure confirmed that neutrophil collagenase is a member of the family of matrix metalloproteinases (e.g., MMP1) but distinct from other members of the family. Neutrophil collagenase shows a preference for type I collagen in contrast with the greater susceptibility of type III collagen to digestion by fibroblast collagenase. Devarajan et al. (Devarajan, P.; Mookhtiar, K.; Van Wart, H.; Berliner, N. : Structure and expression of the cDNA encoding human neutrophil collagenase. Blood 77: 2731-2738, 1991) isolated a 2.4-kb cDNA clone encoding human neutrophil collagenase. From its sequence, it was shown to encode a 467-residue protein which exhibited 58% homology to human fibroblast collagenase and had the same domain structure.

MMP9

For some embodiments of the present invention, the target for the inhibitor agent of the present invention may be MMP9.

Background teachings on matrix metalloproteinase 9 (MMP9) have been presented by Victor A. McKusick et al on http://www.ncbi.nlm.nih.gov/Omim. For ease of reference, the following information has been extracted from that source.

“The 72- and 92-kD type IV collagenases are members of a group of secreted zinc metalloproteases which, in mammals, degrade the collagens of the extracellular matrix. Other members of this group include interstitial collagenase and stromelysin. The 72-kD type IV collagenase is secreted from normal skin fibroblasts, whereas the 92-kD collagenase (CLG4B) is produced by normal alveolar macrophages and granulocytes. Both CLG and STMY have 10 exons of virtually identical length, are located on 11q, and are regulated in a coordinate fashion. By hybridization to somatic cell hybrid DNAs, Collier et al. (1991) demonstrated that both CLG4A and CLG4B are situated on chromosome 16. However, St Jean et al. (1995) assigned CLG4B to chromosome 20. They did linkage mapping of the CLG4B locus in 10 CEPH reference pedigrees using a polymorphic dinucleotide repeat in the 5-prime flanking region of the gene. St Jean et al. (1995) observed lod scores of between 10.45 and 20.29 with markers spanning chromosome region 20q11.2-q13.1. Further support for assignment of CLG4B to chromosome 20 was provided by analysis of human/rodent somatic cell hybrids. Both CLG4A and CLG4B have 13 exons and similar intron locations (Huhtala et al., 1991). Due to these similarities, the CLG4B cDNA clone used in the mapping to chromosome 16 may have hybridized to CLG4A rather than to CLG4B on chromosome 20.

The 13 exons of both CLG4A and CLG4B are 3 more than have been found in other members of this gene family. The extra exons encode the amino acids of the fibronectin-like domain which has been found only in the 72- and 92-kD type IV collagenases. The 92-kD type IV collagenase is also known as 92-kD gelatinase, type V collagenase, or matrix metalloproteinase 9 (MMP9); see the glossary of matrix metalloproteinases provided by Nagase et al. (1992). Linn et al. (1996) reassigned MMP9 (referred to as CLG4B by them) to chromosome 20 based on 3 different lines of evidence: screening of a somatic cell hybrid mapping panel, fluorescence in situ hybridization, and linkage analysis using a newly identified polymorphism. They also mapped mouse Clg4b to mouse chromosome 2, which has no known homology to human chromosome 16 but large regions of homology with human chromosome 20.

By targeted disruption in embryonic stem cells, Vu et al. (1998) created homozygous mice with a null mutation in the MMP9/gelatinase B gene. These mice exhibited an abnormal pattern of skeletal growth plate vascularization and ossification. Although hypertrophic chondrocytes developed normally, apoptosis, vascularization, and ossification were delayed, resulting in progressive lengthening of the growth plate to about 8 times normal. After 3 weeks postnatal, aberrant apoptosis, vascularization, and ossification compensated to remodel the enlarged growth plate and ultimately produced an axial skeleton of normal appearance. Transplantation of wildtype bone marrow cells rescued vascularization and ossification in MMP9-null growth plates, indicating that these processes are mediated by MMP9-expressing cells of bone marrow origin, designated chondroclasts. Growth plates from MMP9-null mice in culture showed a delayed release of an angiogenic activator, establishing a role for this proteinase in controlling angiogenesis.”

MMP10

For some embodiments of the present invention, the target for the inhibitor agent of the present invention may be MMP10.

Background teachings on this matrix metalloproteinase have been presented by Victor A. McKusick et al on http://www.ncbi.nlm.nih.gov/Omim. For ease of reference, the following information has been extracted from that source.

Stromelysin is a metalloproteinase related to collagenase (there is about 55% similarity in their amino acid sequences) whose substrates include proteoglycans and fibronectin, but not type I collagen. Stromelysin II is also called matrix metalloproteinase-10, or MMP10. Muller et al. (Muller, D.; Quantin, B.; Gesnel, M. C.; Millon-Collard, R.; Abecassis, J.; Breathnach, R. : The collagenase gene family in humans consists of at least four members. Biochem. J. 253: 187-192, 1988) detected RNAs capable of hybridizing to a rat stromelysin cDNA in 11 of 69 human tumors tested. These studies were undertaken because of the strong likelihood that tumor invasion and metastasis require enzymic degradation of a host interstitial matrix, a concept that is supported by reports of increased proteolytic activities in tumor cells. By molecular cloning of cDNAs to these RNAs, Muller et al. (1988) identified them as a mixture of stromelysin RNA and a transcript of a hitherto undescribed related gene, that of stromelysin II. They also isolated cDNAs corresponding to a more distantly related human gene, the PUMP1 gene.

MMP11

For some embodiments of the present invention, the target for the inhibitor agent of the present invention may be MMP11.

Background teachings on this matrix metalloproteinase have been presented by Victor A. McKusick et al on http://www.ncbi.nlm.nih.gov/Omim. For ease of reference, the following information has been extracted from that source.

The family of matrix metalloproteinases appears to be involved in physiologic and pathologic processes associated with extracellular matrix remodeling such as those that occur in embryonic development, tissue repair, and tumor progression. Matrisian, Stromelysin III, a member of this gene family, is overexpressed in the stromal cells of invasive breast carcinomas but not in the stromal cells surrounding benign breast fibroadenomas. By in situ hybridization, Levy et al. (Levy, A.; Zucman, J.; Delattre, O.; Mattei, M. G.; Rio, M. C.; Basset, P.: Assignment of the human stromelysin 3 (STMY3) gene to the q11.2 region of chromosome 22. Genomics 13: 881-883, 1992.) assigned the STMY3 gene to 22q. Using a panel of somatic cell hybrids containing different segments off 22q, they demonstrated that the STMY3 gene is in band 22q11.2, in close proximity to the BCR gene involved in chronic myeloid leukemia. Both STMY1 and STMY2 are located on chromosome 11. Stromelysin III is also called matrix metalloproteinase-11, or MMP11. The nomenclature of the matrix metalloproteinases, together with symbols and EC numbers, was provided by Nagase et al. (Nagase, H.; Barrett, A. J.; Woessner, J. F., Jr.: Nomenclature and glossary of the matrix metalloproteinases. Matrix Suppl. 1: 421-424, 1992).

MMP12

For some embodiments of the present invention, the target for the inhibitor agent of the present invention may be MMP 12.

Background teachings on this matrix metalloproteinase have been presented by Victor A. McKusick et al on http://www.ncbi.nlm.nih.gov/Omim. For ease of reference, the following information has been extracted from that source.

The matrix metalloproteases (MMPs) are a family of related matrix-degrading enzymes that are important in tissue remodeling and repair during development and inflammation. Abnormal expression is associated with various diseases such as tumor invasiveness, arthritis, and atherosclerosis. MMP activity may also be related to cigarette-induced pulmonary emphysema. Belaaouaj et al. (Belaaouaj, A.; Shipley, J. M.; Kobayashi, D. K.; Zimonjic, D. B.; Popescu, N.; Silverman, G. A.; Shapiro, S. D.: Human macrophage metalloelastase: genomic organization, chromosomal location, gene linkage, and tissue-specific expression. J. Biol. Chem.270: 14568-14575, 1995) described the genomic organization of the HME gene (also symbolized MMP12). The 13-kb gene is composed of 10 exons and shares the highly conserved intron-exon borders of other MMPs. The authors also demonstrated tissue-specific expression in macrophages and stromal cells. They localized the gene to 11q22.2-q22.3 by fluorescence in situ hybridization.

MMP13

For some embodiments of the present invention, the target for the inhibitor agent of the present invention may be MMP13.

Thus, according to this embodiment, the present invention provides a pharmaceutical for use in damaged tissue, such as wound, treatment (e.g. healing); the pharmaceutical comprising a composition which comprises: (a) a growth factor; and an inhibitor agent; and optionally c) a pharmaceutically acceptable carrier, diluent or excipient; wherein the inhibitor agent can inhibit the action of at least one specific adverse protein (e.g. a specific protease) that is upregulated in a damaged tissue, such as a wound, environment; wherein said specific protein is MMP13.

Background teachings on matrix metalloproteinase 13 (MMP13) have been presented by Victor A. McKusick et al on http://www.ncbi.nlm.nih.gov/Omim. For ease of reference, the following information has been extracted from that source.

“Freije et al. (1994) cloned a cDNA coding for a ‘new’ human matrix metalloproteinase (MMP) from a cDNA library derived from a breast tumor. The isolated cDNA contains an open reading frame coding for a polypeptide of 471 amino acids. The predicted protein sequence displays extensive similarity to previously known MMPs and presented all the structural features characteristic of this protein family, including the well-conserved PRCGXPD motif. In addition, it contains in its amino acid sequence several residues specific to the collagenase subfamily (tyr214, asp235, and gly237) and lacks the 9-residue insertion present in the stromelysins. Because of the structural characteristics, Freije et al. (1994) called the new MMP collagenase-3, since it represented the third member of this family, composed of fibroblast (MMP1) and neutrophil (MMP8) collagenases. Pendas et al. (1997) reported that the MMP13 gene contains 10 exons and spans approximately 12.5 kb. The overall gene organization is similar to those of other MMP genes, including MMP1, MMP7, and MMP12.

Freije et al. (1994) expressed the CLG3 cDNA in a vaccinia virus system and found that the recombinant protein was able to degrade fibrillar collagens, providing support to the idea that the isolated cDNA codes for an authentic collagenase. Northern blot analysis of RNA from normal and pathologic tissues demonstrated the existence in breast rumors of 3 different mRNA species, which seemed to be the result of utilization of different polyadenylation sites present in the 3-prime noncoding region of tie gene. By contrast, no CLG3 mRNA was detected either by Northern blot or RNA polymerase chain reaction analysis with RNA from other human tissues, including normal breast, mammary fibroadenomas, liver, placenta, ovary, uterus, prostate, and parotid gland. A possible role for this metalloproteinase in the tumoral process was proposed.

By fluorescence in situ hybridization, Pendas et al. (1995) localized the CLG3 gene (also symbolized MMP13) to 11q22.3. Physical mapping of a YAC clone containing CLG3 revealed that this gene is tightly linked to those genes encoding other matrix metalloproteinases, including fibroblast collagenase (MMP1), stromelysin-1 (MMP3), and stromelysin-2 (MMP10). Further mapping of this region using pulsed field gel electrophoresis showed that the CLG3 gene is located on the telomeric side of the matrix metalloproteinase cluster. Pendas et al. (1995) found the relative order of the loci to be cen—STMY2—CLG1—STMY1—CLG3—tel. Pendas et al. (1996) isolated a 1.5-Mb YAC clone mapping to 11q22. Detailed analysis of this nonchimeric YAC clone ordered 7 MMP genes as follows: cen—MMP8—MMP10—MMP1—MMP3—MMP12—MMP7—MMP13—tel.

Mitchell et al. (1996) concluded that the expression of MMP13 in osteoarthritic cartilage and its activity against type II collagen indicates that the enzyme plays a significant role in cartilage collagen degradation and must, therefore, form part of a complex target for proposed therapeutic interventions based on collagenase inhibition. Reboul et al. (1996) likewise presented data on collagenase-3 expression and synthesis in human cartilage cells and suggested its involvement in human osteoarthritis cartilage pathophysiology.”

MMP14

For some embodiments of the present invention, the target for the inhibitor agent of the present invention may be MMP 14.

Background teachings on matrix metalloproteinase 14 (MMP14) have been presented by Alan Scott et al on http://www.ncbi.nlm.nih.gov/Omim. For ease of reference, the following information has been extracted from that source.

“Matrix metalloproteinases (MMPs) are Zn(2+)-binding endopeptidases that degrade various components of the extracellular matrix (ECM). The MMPs are enzymes implicated in normal and pathologic tissue remodeling processes, wound healing, angiogenesis, and tumor invasion. MMPs have different substrate specificities and are encoded by different genes. Sato et al. (1994) cloned a cDNA for the human gene from a placenta cDNA library (they called the gene MMP-XI and the gene product membrane-type metalloproteinase). The authors noted that the protein was expressed at the surface of invasive tumor cells. Using degenerate PCR, Takino et al. (1995) cloned the entire genomic sequence of this member of the MMP superfamily (see MMP1). The cDNA identified codes for a 582-amino acid protein which shared conserved sequence and a similar domain structure to other MMPs. They noted that the cDNA, termed MMP-X1 by them, had a unique transmembrane domain at the C terminus. Thus, they predicted that MMP-X1 was a membrane spanning protein rather than a secretory protein like the other MMPs. Northern blots showed that MMP-X1 expression was present at varying intensity in almost all tissues examined, but was highest in the placenta.

Mignon et al. (1995) tabulated 11 members of the matrix metalloproteinase family and their chromosomal locations; with 1 exception, the genes encoding them had been mapped. Six of them, including 3 collagenases and 2 stromelysins, had been assigned to 11q. Membrane-type matrix metalloproteinase (MMP14) may be an activator of pro-gelatinase A and is expressed in fibroblast cells during both wound healing and human cancer progression. By isotopic in situ hybridization, Mignon et al. (1995) mapped the MMP14 gene to 14q11-q12.

By gene targeting, Holmbeck et al. (1999) generated mice deficient in the Mmp14 gene, which they called MT1-MMP. Mmp14 deficiency caused craniofacial dysmorphism, arthritis, osteopenia, dwarfism, and fibrosis of soft tissues due to ablation of a collagenolytic activity that is essential for modeling of skeletal and extraskeletal connective tissues. These findings demonstrated the pivotal function of MMP14 in connective tissue metabolism and illustrated that modeling of the soft connective tissue matrix by resident cells is essential for the development and maintenance of the hard tissues of the skeleton.”

MMP15

For some embodiments of the present invention, the target for the inhibitor agent of the present invention may be MMP 15.

Background teachings on this matrix metalloproteinase have been presented by Victor A. McKusick et al on http://www.ncbi.nlm.nih.gov/Omim. For ease of reference, the following information has been extracted from that source.

Will and Hinzmann (Will, H.; Hinzmann, B.: cDNA sequence and mRNA tissue distribution of a novel human matrix metalloproteinase with a potential transmembrane segment. Europ. J. Biochem. 231: 602-608, 1995) isolated a cDNA encoding a novel MMP (MMP15) from a human lung cDNA library. The MMP15 cDNA encodes a 669-amino acid protein that has the typical structural features of an MMP. In addition, it contains a predicted transmembrane segment at the C terminus. MMP15 shares 73.9% sequence similarity with MMP14, a membrane-localized MMP that also contains a C-terminal transmembrane segment.

MMP16

For some embodiments of the present invention, the target for the inhibitor agent of the present invention may be MMP 16.

Background teachings on this matrix metalloproteinase have been presented by Victor A. McKusick et al on http://www.ncbi.nlm.nih.gov/Omim. For ease of reference, the following information has been extracted from that source.

Takino et al. (Takino, T.; Sato, H.; Shinagawa, A.; Seiki, M.: Identification of the second membrane-type matrix metalloproteinase (MT-MMP-2) gene from a human placenta cDNA library: MT-MMPs form a unique membrane-type subclass in the MMP family. J. Biol. Chem.270: 23013-23020, 1995) isolated a novel MMP cDNA (MMP16) from a human placenta cDNA library. The MMP16 protein consists of 604 amino acids and has a characteristic MMP domain structure. Additionally, MMP16 has a C-terminal extension containing a potential transmembrane domain, similar to MMP14, MMP15, and MMP17.

MMP17

For some embodiments of the present invention, the target for the inhibitor agent of the present invention may be MMP 17.

Background teachings on this matrix metalloproteinase have been presented by Victor A. McKusick et al on http://www.ncbi.nlm.nih.gov/Omim. For ease of reference, the following information has been extracted from that source.

Puente et al. (Puente, X. S.; Pendas, A. M.; Llano, E.; Velasco, G.; Lopez-Otin, C.: Molecular cloning of a novel membrane-type matrix metalloproteinase from a human breast carcinoma. Cancer Res.56: 944-949, 1996.) cloned a cDNA encoding matrix metalloproteinase-17 (MMP17) from a human breast carcinoma cDNA library using degenerate PCR. MMP17, named MT4-MMP by the authors, is a 518-amino acid protein that has a domain organization characteristic of the MMP family, including a prodomain with an activation locus, a zinc-binding site, and a hemopexin domain. MMP17 also has a C-terminal extension that contains a putative transmembrane domain, indicating that it is a member of the membrane-type MMP subclass (see MMP14, MMP15, MMP16).

MMP19

For some embodiments of the present invention, the target for the inhibitor agent of the present invention may be MMP19.

Background teachings on this matrix metalloproteinase have been presented by Victor A. McKusick et al on http://www.ncbi.nlm.nih.gov/Omim. For ease of reference, the following information has been extracted from that source.

Using an MMP similarity search of the EST database, Cossins et al. (Cossins, J.; Dudgeon, T. J.; Catlin, G.; Gearing, A J. H.; Clements, J. M. : Identification of MMP-18, a putative novel human matrix metalloproteinase. Biochem. Biophys. Res. Commun. 228: 494-498, 1996) identified a partial cDNA clone that encodes the 3-prime end of a putative MMP, which they called MMP18 but which has officially designated MMP19. They PCR-amplified the 5-prime end and cloned and sequenced the full-length cDNA. MMP19 contains an open reading frame of 508 amino acids with a predicted molecular weight of 57,238 and has all the characteristic features of the MMP family. MMP18 contains a putative signal sequence, followed by a prodomain with a conserved ‘cysteine switch’ region. Expression of a single transcript of 2.7 kb was detected in placenta, lung, pancreas, ovary, small intestine, spleen, thymus, and prostate, and at much lower levels in testis, colon, and heart. No MMP19 mRNA was detected in brain, skeletal muscle, liver, kidney, or peripheral blood leukocytes.

Inhibitor Agent

An essential component of the composition of the present invention is an inhibitor agent. The inhibitor agent may be any suitable agent that can act as an inhibitor of a respective protein (e.g. protease) that is upregulated in a damaged tissue, such as a wound, environment—wherein the protein (protease) has an adverse (deleterious) effect on the healing of damaged tissue.

The term “inhibitor” as used herein with respect to the agent of the present invention means an agent that can reduce and/or eliminate and/or mask and/or prevent the action of a respective protein (e.g. protease) that is upregulated in a damaged tissue, such as a wound, environment—wherein the protein (proteases) has an adverse (deleterious) effect on the healing of damaged tissue.

Particular inhibitor agents include one or more suitable members of: an inhibitor of uPA (I:uPA), an inhibitor of MMP1 (I:MMP1), an inhibitor of MMP2 (I:MMP2), an inhibitor of MMP3 (I:MMP3), an inhibitor of MMP7 (I:MMP7), an inhibitor of MMP8 (I:MMP8), an inhibitor of MMP9 (I:MMP9), an inhibitor of MMP10 (I:MMP10), an inhibitor of MMP11 (I:MMP11), an inhibitor of MMP12 (I:MMP12), an inhibitor of MMP13 (I:MMP13), an inhibitor of MMP14 (I:MMP14), an inhibitor of MMP9 (I:MMP15), an inhibitor of MMP16 (I:MMP16), an inhibitor of MMP17 (I:MMP17), an inhibitor of MMP19 (I:MMP19) an inhibitor of MMP20 (I:MMP20), an inhibitor of MMP21 (I:MMP21), an inhibitor of MMP24 (I:MMP24), an inhibitor of MMPFMF(I:MMPFMF).

The inhibitor agent can be an amino acid sequence or a chemical derivative thereof. The substance may even be an organic compound or other chemical. The agent may even be a nucleotide sequence—which may be a sense sequence or an anti-sense sequence. The agent may be an antibody. For some applications, preferably, the inhibitor agent is a synthetic organic molecule.

Thus, the term “inhibitor” includes, but is not limited to, a compound which may be obtainable from or produced by any suitable source, whether natural or not. The inhibitor may be designed or obtained from a library of compounds which may comprise peptides, as well as other compounds, such as small organic molecules, such as lead compounds.

By way of example, the inhibitor may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

As used herein, the term “inhibitor” may be a single entity or it may be a combination of agents. Hence, the inhibitor agent of the composition of the present invention may be two or more agents that are capable of inhibiting the action of one or more proteins that are upregulated in a damaged tissue, such as a wound, environment. Thus, the composition of the present invention may comprise an I:uPA and an I:MMP. In another embodiment, the composition of the present invention may comprise an I:uPA and an I:MMP1 and/or an I:MMP2 and/or an I:MMP3 and/or an I:MMP7 and/or an I:MMP8 and/or an I:MMP9 and/or an I:MMP10 and/or an I:MMP11 and/or an I:MMP12 and/or an I:MMP13 and/or an I:MMP14 and/or an I:MMP15 and/or an I:MMP16 and/or an I:MMP17 and/or an I:MMP19 and/or an I:MMP20 and/or an I:MMP21 and/or an I:MMP24 and/or an I:MMPFMF. In another embodiment, the composition of the present invention may comprise a first I:uPA and a second I:uPA and/or a first I:MMP and/or a second I:MMP.

The inhibitor agent of the composition of the present invention may comprise one agent that is capable of inhibiting the action of two or more proteins that are upregulated in a damaged tissue, such as a wound, environment. Thus, the composition of the present invention may comprise an agent that is capable of acting as an I:uPA and an I:MMP. In another embodiment, the composition of the present invention may comprise an agent that is capable of acting as an I:uPA and an I:MMP1 and/or an I:MMP2 and/or an I:MMP3 and/or an I:MMP7 and/or an I:MMP8 and/or an I:MMP9 and/or an I:MMP10 and/or an I:MMP11 and/or an I:MMP12 and/or an I:MMP13 and/or an I:MMP14 and/or an I:MMP15 and/or an I:MMP16 and/or an I:MMP17 and/or an I:MMP19 and/or an I:MMP20 and/or an I:MMP21 and/or an I:MMP24 and/or an I:MMPFMF.

The inhibitor agent of the present invention may even be capable of displaying other therapeutic properties.

The inhibitor agent may be used in combination with one or more other pharmaceutically active agents.

If a combination of active agents are administered, then they may be administered simultaneously, separately or sequentially.

Stereo and Geometric Isomers

Some of the specific inhibitor agents and/or growth factors may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

Pharmaceutical Salt

The inhibitor agent of the present invention—and possibly the growth factor of the present invention—may be administered in the form of a pharmaceutically acceptable salt.

Pharmaceutically-acceptable salts are well known to those skilled in the art, and for example include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an inhibitor agent of the present invention may be readily prepared by mixing together solutions of the agent and the desired acid or base, as appropriate. The salt may precipitate from solution and be collected by filtration or may be recovered by evaporation of the solvent.

The inhibitor agent of the present invention may exisit in polymorphic form.

The inhibitor agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

It will be appreciated by those skilled in the art that the agent of the present invention may be derived from a prodrug. Examples of prodrugs include entities that have certain protected group(s) and which may not possess pharmacological activity as such, but may, in certain instances, be administered (such as orally or parenterally) and thereafter metabolised in the body to form the agent of the present invention which are pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of the agents. Such prodrugs are also included within the scope of the invention.

The present invention also includes (wherever appropriate) the use of zwitterionic forms of the inhibitor agent of the present invention—and possibly the growth factor of the present invention.

The terms used in the claims encompass one or more of the forms just mentioned.

Solvates

The present invention also includes the use of solvate forms of the inhibitor agent of the present invention—and wherever applicable the growth factor of the present invention. The terms used in the claims encompass these forms.

Pro-Drug

As indicated, the present invention also includes the use of pro-drug forms of the inhibitor agent of the present invention—and wherever applicable the growth factor of the present invention. The terms used in the claims encompass these forms.

Chemical Synthesis Methods

Typically the inhibitor agent of the present invention will be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts of the invention may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

The inhibitor agent or growth factor of the present invention or variants, homologues, derivatives, fragments or mimetics thereof may be produced using chemical methods to synthesize the agent in whole or in part. For example, if they are peptides, then peptides can be synthesized by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Syntesis of peptide inhibitor agents or of the growth factors (or variants, homologues, derivatives, fragments or mimetics thereof) can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent or growth factor.

In an alternative embodiment of the invention, the coding sequence of a peptide inhibitor agent or growth factor (or variants, homologues, derivatives, fragments or mimetics thereof) may be synthesized, in whole or in part, using chemical methods well known in the art (see Caruthers M H et at (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the inhibitor agent may be a chemically modified inhibitor agent.

The chemical modification of an agent of the present invention may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Recombinant Methods

The growth factor of the present invention may be prepared by recombinant DNA techniques.

Urokinase Inhibitor

A component of the composition of the present invention may be an inhibitor of urokinase-type plasminogen activator. Typically, the I:uPA will be capable of being identified as being an I:uPA by a uPA assay—such as the assay protocol presented herein.

Thus, in one aspect, the present invention relates to a method of enhancing the healing of chronic dermal ulcers, including venous stasis ulcers, diabetic ulcers and decubitus ulcers (or pressure sores), by treating the patient with a combination of a selective inhibitor of uPA and a growth factor. This combination therapy is more effective than treatment with the individual agents.

The inhibitors of uPA can either be applied topically or administered orally, depending on the properties of the inhibitor and the way in which they are formulated.

Thus, according to one aspect of the present invention, the composition may comprise an I:uPA—such as a selective uPA inhibitor—and a growth factor. With the co-administration of these two components a more profound efficacy can be achieved than by administration of either a growth factor or a uPA inhibitor alone. Here, efficacy may be measured by the standard of the FDA in this area—such as the time to closure of chronic dermal ulcers under conditions of best care and compared to best care alone.

In one preferred aspect, topical formulations of selective uPA inhibitors can be co-administered with topically administered growth factors, such as PDGF, either by physically mixing the substances and using a formulation which releases both substances into the damaged tissue, such as a wound, environment, or by applying one substance at a time and using a treatment protocol which separates application of the agents. Alternatively, combined treatment can be achieved using an orally administered uPA inhibitor with topical application of a growth factor.

We believe that the use of I:uPA when co-administered with growth factors is very advantageous and was, also, unexpected and unpredictable. In this respect, many literature reports show that uPA is required as part of the signalling cascade downstream from growth factor receptors. We have determined that, whilst this may be the case, the protective effects of a selective uPA inhibitor on growth factors, and cellular responses to growth factors, predominates.

In accordance with the present invention, the I:uPA may be applied topically mixed with the growth factor or the I:uPA may be applied topically but at a different time to the growth factor or the I:uPA may be administered orally and the growth factor may be applied topically.

The I:uPA may be naturally occurring or it may be a synthetic entity.

A number of I:uPAs are known. For example, reference may be made to C. Magill et al. Emerg. Therap. Targets 1999, 3(1), 109-133, and H. Yang et al. Fibrinolysis 1992, 6 (Suppl 1), 31-34.

Examples of naturally occurring proteinacious inhibitors include plasminogen activator inhibitor proteins PAI-1 and PAI-2 (see Antalis, T. M., Clark, M. A., Barnes, T., Lehrbach, P. R., Devine, P. L., Schevzov, G., Goss, N. H., Stephens, R. W. & Tostoshev, P. (1988) Proc. Natl. Acad. Sci. U.S.A. 85, 985-999). Reference may also be made to WO 99/49887. Another naturally occurring proteinacious inhibitor is □-antitrypsin.

Other naturally naturally occurring inhibitors include ε-Aminocaproic acid (ε-aca)—which is a weak inhibitor. Vitamin E (α-tocopherol) is an irreversible inhibitor of urokinase which acts via an unknown mechanism. Natural catechols isolated from green tea such as epigallocathechin-3 gallate (EGCG) inhibit urokinase. The nortriterpenoid demethylzeylasteral (TZ-93) isolated from Tripterygium wilfordii inhibits urokinase activity. The protein aprotinin is a weak inhibitor of urokinase but not t-PA.

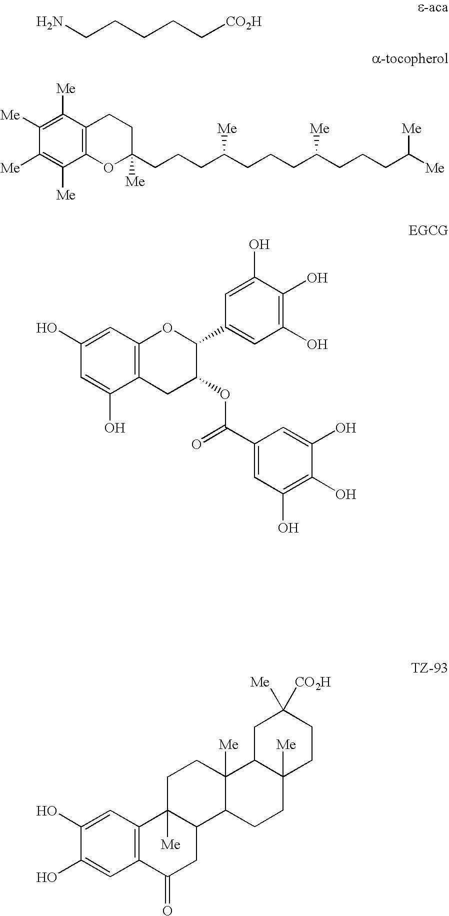

In addition, synthetic inhibitors of uPA exist. These synthetic inhibitors will typically be organic compounds. Typically the organic compounds will comprise a guanidine group (i.e.—N═C(NH₂)(NH₂)) and one or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group, wherein that cyclic group is a polycyclic group, preferably being a fused polycyclic group—such as an isoquinoline group. For some applications, preferably the guanidine group is attached to said hydrocarbyl group. For some applications, the agent comprises at least the one of said cyclic groups linked to another hydrocarbyl group, which other hydrocarbyl group has an ester group, an acid group or an alkoxy group thereon.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The I:uPAs may have a reversible or irreversible action.

Reported irreversible inhibitors generally rely on forming a covalent bond with the active site serine (Ser-195) which forms part of the catalytic triad of urokinase. Camostat (FOY-05) and its more plasma stable metabolite (FOY-251) are potent trypsin inhibitors which were found to inhibit urokinase irreversibly at nanomolar concentrations. Arginyl chloromethylketones also bind and inactivate urokinase with Glu-Gly-Arg-CH₂Cl being the best inhibitor. Cyclic peptide (methyl)phenylsulfonium (1) inhibits urokinase along with bovine trypsin and, to a lesser degree t-PA.

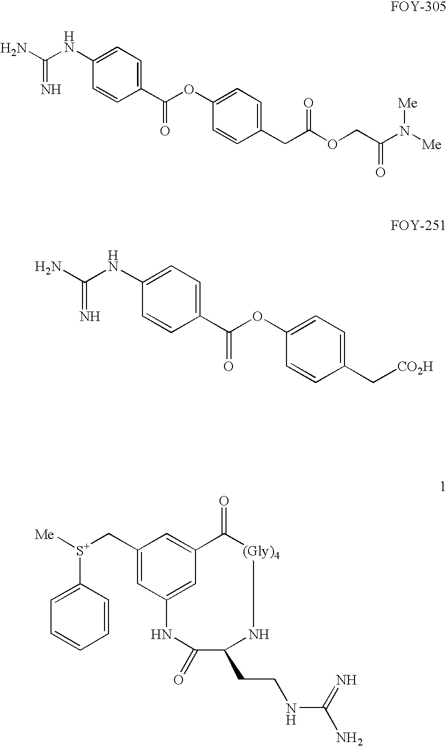

The benzothiazole ketone MOL-174 is a potent inhibitor of thrombin which also demonstrates affinity for urokinase. The peptidic boronate (2) is a competitive inhibitor of urokinase. Phenylalanine derived structues (e.g. 3) were also shown to inhibit urokinase. CVS-3083 is a potent inhibitor of urokinase. CVS-3083 is an arginyl aldehyde which acts as a transition state mimic by forming a reversible covalent bond with Ser-195. Plasma kallikrein selective inhibitor (PKSI-527) weakly inhibits urokinase.

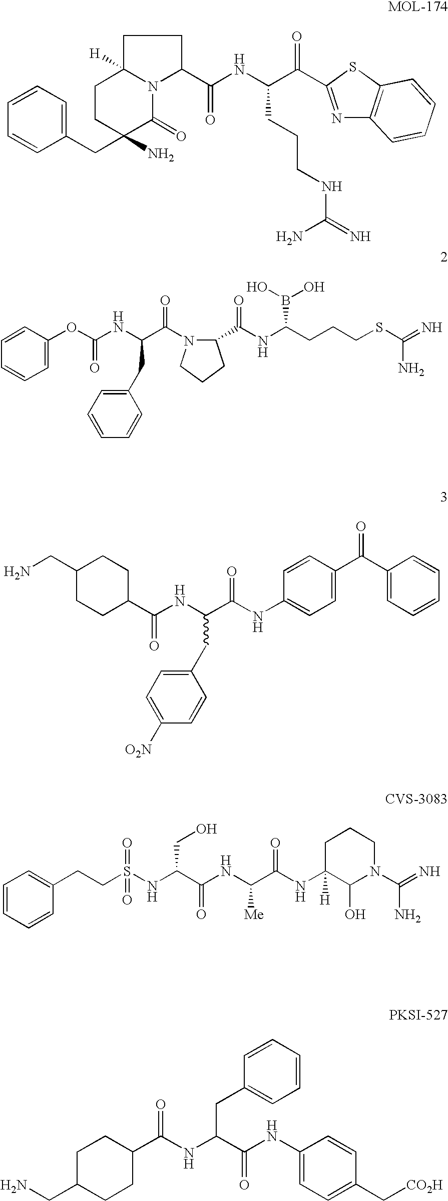

Following the discovery of ε-aca, a number of aromatic and heterocyclic amidines were reported as urokinase inhibitors (e.g. 4-9). Bis-(5-amidino-benzimidazolyl)methane (BABIM; 8) was one of the more potent, but was poorly selective over other trypsin-like serine proteases.

Another inhibitor that may be used is Nafamostat (FUT-175) which can inhibit various serine proteases, including urokinase. However, for some embodiments the inhibitor is not Nafamnostat since the selectivity may not be great as desired for some applications.

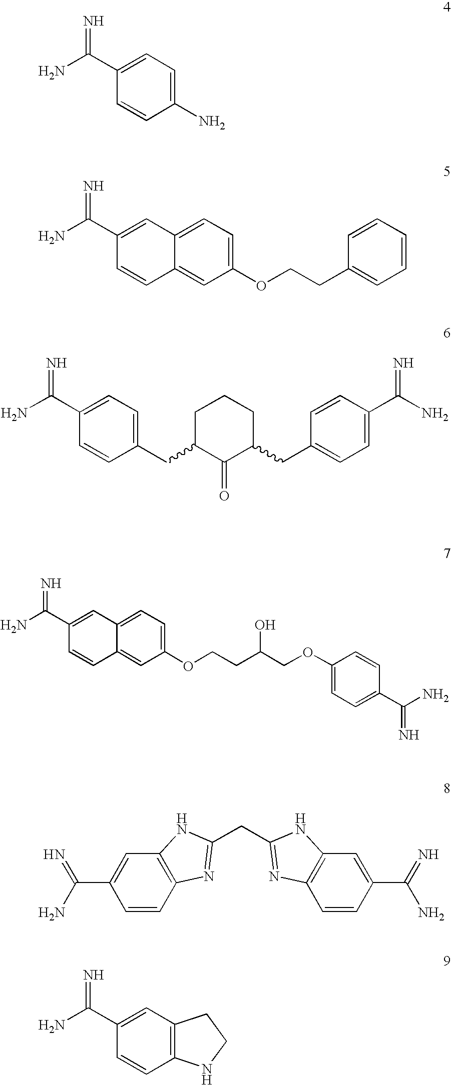

Aromatic guanidines have also been reported as urokinase inhibitors. The diuretic drug amiloride™ is an inhibitor of urokinase. Simple phenyl guanidines such as 4-chloro and 4-(trifluoromethyl)phenylguanidine (10 and 11 respectively) are selective inhibitors of urokinase.

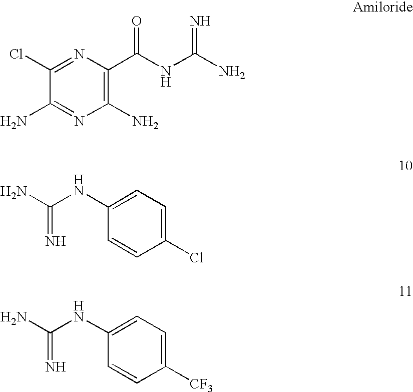

Bridges et al. reported a series of benzothiophenes and thienothiophenes as urokinase inhibitors [see EP-A-0568289]. Compounds of formula I were mentioned, e.g. B428 (Ia) and B623 (Ib).

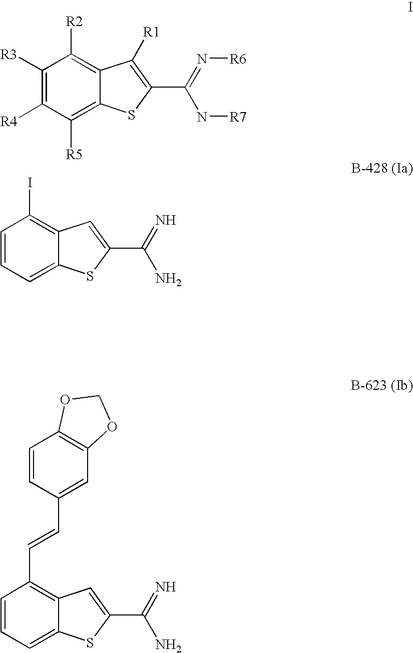

Specific examples are: 4-iodobenxo[b]thiophene-2-carboxamidine (Ia); 4-[5-(4-carboxamidinophenyl)fur-2-yl]benzo[b]thiophene-2-carboxamidine; 4-[E/Z-2-(benzo-1,3-dioxolan-5-yl)ethyl]benzo[b]thiophene-2-carboxamidine (Ib); and 4-[(benzo-1,3-dioxolan-5-yl)ethynyl]benzo[b]thiophene-2-carboxamidine.

Tanaka et al. reported a series of 4,5,6,7-tetrahydrobenzo[b]thiophenes as urokinase inhibitors [see WO-A-98/11089]. Compounds of the Formula II, e.g. IIa, were mentioned.

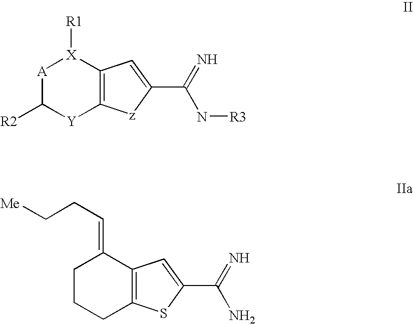

A specific example is: 2-amidino-4-n-butyl-4,5,6,7-tetrahydrobenzo[b]thiophene (IIa).

Greyer et al. reported a series of 2-amidinonaphthalenes as urokinase inhibitors [see WO-A-99/05096]. Compounds of formula III were mentioned, e.g. IIIa.

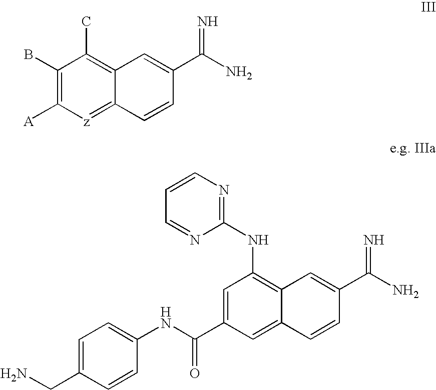

Specific examples are: 6-(aminoiminomethyl)-N-[4-(aminomethyl)phenyl]-4-(2-pyrimidinylamino)-2-naphthalenecarboxamide (IIIa); 6-(aminoiminomethyl)-N-[4-(hydroxymethyl)phenyl]-4-(2-pyrimidinylamino)-2-naphthalenecarboxamide; 6-(aminoiminomethyl)-N-phenyl-4-(2-pyrimidinylamino)-2-naphthalenecarboxamide; and methyl [7-(aminoiminomethyl)-3-[[[4-(aminomethyl)phenyl]amino]carbonyl]-1-naphthalenyl]carbamate.

Illig et al. reported heteroaryl amidines, methylamidines and guanidines as protease inhibitors, in particular as urokinase inhibitors [see WO-A-99/40088]. Compounds of the general formula IV, e.g. IVa, were mentioned.

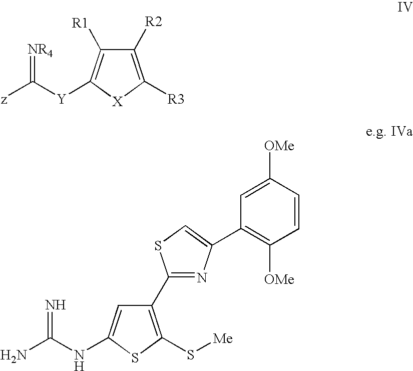

Specific examples are: 4-[4-(2,5-dimethoxyphenyl)(1,3-thiazol-2-yl)]-5-methylthiothiophene-2-carboxamidine (IVa); 2-{3-[2-(5-amidino-2-methylthio-3-thienyl)-1,3-thiazol-4-yl]phenoxy}acetic acid; and 5-methylthio-4-{4-[3-(2-oxo-2-piperazinylethoxy)phenyl](1,3-thiazol-2-yl)}thiophene-2-carboxamidine.

Schirlin et al. reported ketone bearing peptidase inhibitors for inhibiting e.g. urokinase [see U.S. Pat. No. 5849866]. Ketone-bearing inhibitors of generic formula V are new. Specific urokinase inhibitors include Va.
R₁NH—CHR₂—C(O)—X V
H—Glu—Gly—Arg—COOH Va

Barber et al. reported isoquinolines as urokinase inhibitors [see WO-A-99/20608]. Compounds of formula VI were disclosed, e.g. VIa.

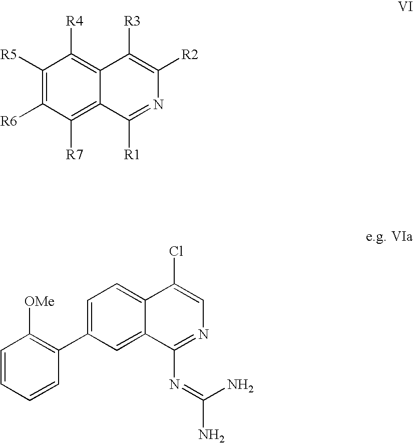

In more detail, the compounds of WO-A-99/20608 are isoquinolinylguanidine derivatives of formula (I) 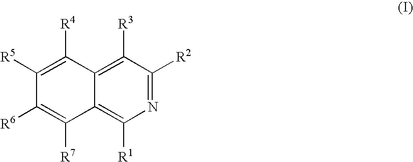
or a pharmaceutically acceptable salt thereof, wherein
one of R¹ and R² is H and the other is N═C(NH₂)₂ or NHC(═NH)NH₂,
R³ is H, halogen, C₁₋₆ alkyl optionally substituted by one or more halogen, or C₁₋₆ alkoxy optionally substituted by one or more halogen,
R⁴, R⁵, R⁶ and R⁷ are each independently H, OH, halogen, C₁₋₆ alkyl optionally substituted by one or more substituents independently selected from halogen or OH, C₁₋₆ alkoxy optionally substituted by one or more halogen, CN, CO(C₁₋₆ alkyl optionally substituted by one or more halogen), (Cₘ-alkylene)CO₂R⁸, (Cₙ-alkylene)CN, O(Cₙ-alkylene)CN, O(Cₙ-alkylene)CO₂R⁸, (Cₘ-alkylene)CONR⁹R¹⁰, (Cₘ-alkylene)NR⁹COR¹⁰, O(Cₙ-alkylene)CONR⁹R¹⁰, (Cₘ-alkylene)NR⁹SO₂R¹¹, (Cₘ-alkylene)S(O)ₚR¹¹, (Cₘ-alkylene)SO₂NR⁹R¹⁰, CH═CHCOR⁸, CH═CHCONR⁹R¹⁰, CH═CHSO₂R⁸, CH═CHSO₂NR⁹R¹⁰, CH═CHSO₂aryl, or a group of formula X-aryl or X-het, or, where two of R⁴, R⁵, R⁶ and R⁷ are attached to adjacent carbon atoms, they can be taken together to form an —O(Cₙ-alkylene)O-moiety,
R⁸ is H, C₁₋₆ alkyl optionally substituted by one or more halogen, or aryl(C₁₋₆ alkylene),
R⁹ and R¹⁰ are each independently H, C₁₋₆ alkyl optionally substituted by one or more halogen, aryl(C₁₋₆ alkylene), aryl, heteroaryl or heteroaryl(C₁₋₆ alkylene), or R⁹ and R¹⁰ may be linked together by an alkylene moiety to form, with the atoms to which they are attached, a 4- to 7-membered ring optionally incorporating an additional hetero-group selected from an 0 or S atom or a NR¹² group,
R¹¹ is aryl, heteroaryl, or C₁₋₆ alkyl optionally substituted by one or more halogen,
R¹² is H, C₁₋₆ alkyl optionally substituted by one or more halogen, or CO(C₁₋₆ alkyl optionally substituted by one or more halogen),
X is a direct link, Cₙ-alkylene, O, (Cₙ-alkylene)O, O(Cₙ-alkylene), CH(OH), C(Cₙ-alkyl)OH, CO, S(O)ₚ(Cₘ-alkylene), (Cₘ-alkylene)S(O)ₚ, CH═CH, or C═C,
“aryl” is phenyl or naphthyl optionally substituted by one or more substituents independently selected from halogen, C₁₋₆ alkyl optionally substituted by one or more substituents independently selected from halogen and OH, C₁₋₆ alkoxy optionally substituted by one or more halogen, CN, O(Cₙ-alkylene)CN, (Cₙ-alkylene)CN, CO(C₁₋₆ alkyl optionally substituted by one or more halogen), (Cₘ-alkylene)CO₂R¹³,O(Cₙ-alkylene)CO₂R¹³, (Cₘ-alkylene)CONR¹⁴R¹⁵, (Cₘ-alkylene)NR¹⁴COR¹⁵, O(Cₙ-alkylene)CONR¹⁴R¹⁵, (Cₘ-alkylene)S(O)ₚR¹³, (Cₘ-alkylene)SO₂NR¹⁴R¹⁵, (Cₘ-alkylene)NR¹⁴SO₂R¹⁶, CH═CHSO₂R¹³, CH═CHSO₂NR¹⁴R¹⁵, CH═CHSO₂aryl¹, CH═CHCOR¹³, and CH═CHCONR¹⁴R¹⁵,
“heteroaryl” is an optionally benzo-fused 5- or 6-membered heterocyclic group linked by any available atom in the heterocyclic or benzo-ring (if present), which heterocyclic group is selected from dioxolyl, furyl, thienyl, pyrrolyl, oxazolyl, thiazolyl, isoxazolyl, isothiazolyl, imidazolyl, pyrazolyl, oxadiazolyl, thiadiazolyl, triazolyl, tetrazolyl, pyridyl, pyrimidinyl, pyridazinyl, pyrazinyl and pyranyl,
said “heteroaryl” group being optionally substituted by one or more substituents independently selected from halogen, C₁₋₆ alkyl optionally substituted by one or more substituents independently selected from halogen or OH, C₁₋₆ alkoxy optionally substituted by one or more halogen, CN, O(Cₙ-alkylene)CN, (Cₙ-alkylene)CN, CO(C₁₋₆ alkyl optionally substituted by one or more halogen), (Cₘ-alkylene)CO₂R¹³, O(Cₙ-alkylene)CO₂R¹³, (Cₘ-alkylene)CONR¹⁴R¹⁵, (Cₘ-alkylene)NR¹⁴COR¹⁵, O(Cₙ-alkylene)CONR¹⁴R¹⁵, Cₘ-alkylene)NR¹⁴SO₂R¹⁶, (Cₘ-alkylene)S(O)ₚR¹³, (Cₘ-alkylene)SO₂NR¹⁴R¹⁵, CH═CHCOR¹³, CH═CHCONR¹⁴R¹⁵, CH═CHSO₂R¹³, CH═CHSO₂NR¹⁴R¹⁵, or CH═CHSO₂aryl¹,
“het” is an optionally benzo-fused 5- or 6-membered heterocyclic group linked to the “X” moiety by any available atom in the heterocyclic or benzo-ring (if present), which heterocyclic group is selected from dioxolyl, dioxolanyl, furyl, thienyl, pyrrolyl, oxazolyl, oxazinyl, thiazinyl, thiazolyl, isoxazolyl, isothiazolyl, imidazolyl, pyrazolyl, oxadiazolyl, thiadiazolyl, triazolyl, tetrazolyl, pyridyl, pyrimidinyl, pyridazinyl, pyrazinyl and pyranyl,
or a fully unsaturated, partially or fully saturated analogue thereof,
such “het” group being optionally substituted by one or more substituents independently selected from halogen, C₁₋₆ alkyl optionally substituted by one or more substituents independently selected from halogen and OH, C₁₋₆ alkoxy optionally substituted by one or more halogen, CN, O(Cₙ-alkylene)CN, (Cₙ-alkylene)CN, CO(C₁₋₆ alkyl optionally substituted by one or more halogen), (Cₘ-alkylene)CO₂R¹³, O(Cₙ-alkylene)CO₂R¹³, (Cₘ-alkylene)CONR¹⁴R¹⁵, (Cₘ-alkylene)NR¹⁴COR¹⁵, O(Cₙ-alkylene)CONR¹⁴R¹⁵, (Cₘ-alkylene)NR¹⁴SO₂R¹⁶, (Cₘ-alkylene)S(O)ₚR¹³, (Cₘ-alkylene)SO₂NR¹⁴R¹⁵, CH═CHCOR¹³, CH═CHCONR¹⁴R¹⁵, CH═CHSO₂R¹³, CH═CHSO₂NR¹⁴R¹⁵, and CH═CHSO₂aryl¹,
“aryl¹” is phenyl or naphthyl optionally substituted by one or more substituents independently selected from halogen, C₁₋₆ alkyl optionally substituted by one or more substituents independently selected from halogen or OH, C₁₋₆ alkoxy optionally substituted by one or more halogen, CN, O(Cₙ-alkylene)CN, (Cₙ-alkylene)CN, CO(C₁₋₆ alkyl optionally substituted by one or more halogen), (Cₘ-alkylene)CO₂R¹³, O(Cₙ-alkylene)CO₂R¹³, (Cₘ-alkylene)CONR¹⁴R¹⁵, (Cₘ-alkylene)NR¹⁴COR¹⁵, O(Cₙ-alkylene)CONR¹⁴R¹⁵, (Cₘ-alkylene)S(O)ₚR¹³, (Cₘ-alkylene)SO₂NR¹⁴R¹⁵, (Cₘ-alkylene)NR¹⁴SO₂R¹⁶, CH═CHSO₂R¹³, CH═CHSO₂NR¹⁴R¹⁵, CH═CHCOR¹³, and CH═CHCONR¹⁴R¹⁵,
R¹³ is H, C₁₋₆ alkyl optionally substituted by one or more halogen, or aryl²(C₁₋₆ alkylene),
R¹⁴ and R¹⁵ are each independently H, C₁₋₆ alkyl optionally substituted by one or more halogen, aryl²(C₁₋₆ alkylene), aryl², heteroaryl¹ or heteroaryl¹(C₁₋₆ alkylene),
or R⁹ and R¹⁰ may be linked together by an alkylene moiety to form, with the atoms to which they are attached, a 4- to 7-membered ring optionally incorporating an additional hetero-group selected from an O or S atom or a NR¹² group,
R¹⁶ is aryl², heteroaryl¹, or C₁₋₆ alkyl optionally substituted by one or more halogen,
“aryl²” is phenyl or naphthyl optionally substituted by one or more substituents independently selected from halogen, C₁₋₆ alkyl optionally substituted by one or more substituents independently selected from halogen or OH, C₁₋₆ alkoxy optionally substituted by one or more halogen, CN, O(Cₙ-alkylene)CN, (Cₙ-alkylene)CN, or CO(C₁₋₆ alkyl optionally substituted by one or more halogen),
“heteroaryl” is an optionally benzo-fused 5- or 6-membered heterocyclic group linked by any available atom in the heterocyclic or benzo-ring (if present), which heterocyclic group is selected from dioxolyl, furyl, thienyl, pyrrolyl, oxazolyl, thiazolyl, isoxazolyl, isothiazolyl, imidazolyl, pyrazolyl, oxadiazolyl, thiadiazolyl, triazolyl, tetrazolyl, pyridyl, pyrimidinyl, pyridazinyl, pyrazinyl and pyranyl,
said “heteroaryl¹” group being optionally substituted by one or more substituents independently selected from halogen, C₁₋₆ alkyl optionally substituted by one or more substituents independently selected from halogen or OH, C₁₋₆ alkoxy optionally substituted by one or more halogen, CN, O(Cₙ-alkylene)CN, (Cₙ-alkylene)CN, or CO(C₁₋₆ alkyl optionally substituted by one or more halogen),
wherein the “C-alkylene” linking groups in the definitions above are linear or branched, and are optionally substituted by one or more (C₁₋₆ alkyl optionally substituted by one or more halogen) groups,
m is an integer from 0 to 3, n is an integer from 1 to 3, and p is an integer from 0 to 2.

The most preferred compounds are selected from:
(4-chloro-7-(2-methoxyphenyl)isoquinolin-1-yl)guanidine (VIa);
(4-chloro-7-(3-methoxyphenyl)isoquinolin-1-yl)guanidine;
(4-chloro-7-(4-methoxyphenyl)isoquinolin-1-yl)guanidine;
(4-bromo-7-(3-methoxyphenyl)isoquinolin-1-yl)guanidine;
(4-bromo-7-(4-methoxyphenyl)isoquinolin-1-yl)guanidine;
(4-chloro-7-(□-hydroxybenzyl)isoquinolin-1-yl)guanidine;
(4-chloro-7-(3-carboxyphenyl)isoquinolin-1-yl)guanidine;
1-guanidino-7-sulphamoylisoquinoline;
1-guanidino-7-phenylsulphamoylisoquinoline;
4-chloro-1-guanidino-7-sulphamoylisoquinoline;
4-chloro-7-cyclopentylsulphamoyl-1-guanidinoisoquinoline;
4-chloro-1-guanidino-7-(1-pyrrolidinosulphonyl)isoquinoline;
4-chloro-1-guanidino-7-morpholinosulphonylisoquinoline;
4-chloro-1-guanidino-7-[(N-methylpiperazino)sulphonyl]isoquinoline;
4-chloro-1-guanidino-7-(phenylsulphanyl)isoquinoline; and
4-chloro-1-guanidino-7-(phenylsulphonyl)isoquinoline.

Another preferred compound disclosed in WO-A-99/20608 for use in the present invention is (4-chloro-7-(2,6-dimethoxyphenyl)isoquinolin-1-yl)guanidine-viz: 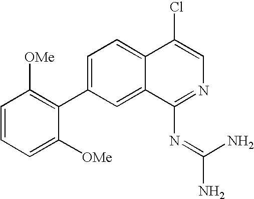
which can be prepared by the method reported in WO-A-99/20608 (see Example 39).

Another preferred compound disclosed in WO-A-99/20608 for use in the present invention is [7-(3-Carboxyphenyl)-4-chloroisoquinolin-1-yl]guanidine-viz: 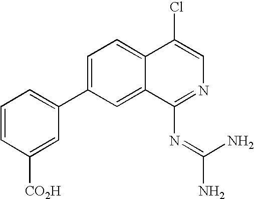
which can be prepared by the method reported in WO-A-99/20608 (see Example 55).

Suitable I:uPA compounds for use in the present invention are disclosed in PCT patent application No. PCT/IB99/01289 (incorporated herein by reference), which was filed on 15 Jul., 1999 (published as WO-A-00/05214). Claiming priority dates of 24 Jul. 1998 and 16 Apr. 1999 Some relevant teachings of that patent application are provided herein (see the section titled “PCS9494 Compounds”).

Preferred compounds from WO-A-00/05214 are presented as Examples 32b therein (hereinafter referred to as “compound 5214”. The formula for Compound 5214 is presented in the Examples section. Another preferred compound from WO-A-00/05214 is Example 34b therein.

Other suitable I:uPA compounds for use in the present invention are disclosed in GB patent application No. 9908410.5 which was filed on 13 Apr. 1999 (incorporated herein by reference) and in U.S. patent application Ser. No. 09/546410 (incorporated herein by reference) and European patent application No. 00302778.6 (incorporated herein by reference) and in Japanese patent application No. 2000-104725 (incorporated herein by reference). Some relevant teachings of those patent applications are provided herein (see the section titled “PCS9482 Compounds”).

Urokinase Inhibitor Assay Protocol

The following presents a protocol for identifying one or more agents capable of acting as an I:uPA that would be suitable for use in the composition of the present invention.

Materials

uPA (urokinase type plasminogen activator). High molecular weight human urokinase from urine, 3000 IU/vial (Calbiochem, 672081) reconstituted in H₂O to give 30000 IU/ml stock and stored frozen (−18° C.). S-2444, chromogenic urokinase substrate, 25 mg/vial (Quadratech, 820357) was reconstituted in H₂O to give 3 mM stock and stored at 4° C. Human tPA stimulator (Chromogenix 822130-63/9) was reconstituted to 1 mg/ml in buffer; and used fresh. Human tPA (one chain) 10 μg/vial (Chromogenix, 821157-039/0) was reconstituted to 4 μg/ml in buffer and used fresh. S-2288, chromogenic substrate for serine proteases, 25 mg/vial (Chromogenix, 820852-39) was reconstituted in H₂O to give 10 mM stock and stored at 4° C. Human plasmin, 2 mg/vial (Quadratech, 810665) was reconstituted to 1 mg/ml in buffer and stored frozen (−18° C.). Chromozym-PL (Boehringer Mannheim, 378 461), 1 mM stock in buffer prepared fresh.

Methods

Chromogenic assays are performed to measure uPA, tPA and plasmin activity and inhibition of this activity by serine protease inhibitors.

IC₅₀ and Kᵢ values for compounds are calculated by incubation of 33 IU/ml uPA with 0.18 mM S2444 (substrate) and various compound concentrations, all diluted in uPA assay buffer (75 mM Tris, pH 8.1, 50 mM NaCl). A pre-incubation of compound with enzyme is carried out for 15 minutes at 37° C., followed by substrate addition and further incubation for 30 minutes at the same temperature. The final assay volume is 200□1. Absorbance is read at 405 nM following pre-incubation (background, time zero measurement) and following the 30 minute incubation with substrate using a SPECTRAMax microplate reader (Molecular Devices Corporation. Background values are subtracted from the final absorbance values. Percentage inhibition is calculated and plotted against compound concentration to generate IC₅₀ values. The enzymatic Kᵢ is calculated from the known Kₘ of the substrate, 90 μM, using the equation Kᵢ═IC₅₀/((1+([S]/Kₘ)).

The method for analysis of tPA inhibition is similar to that for uPA inhibition. The assay utilises final concentrations of tPA of 0.4 □g/ml with 0.1 mg/ml tPA stimulator, 0.4 mM S2288 (substrate) and various concentrations of inhibitors, made up in uPA assay buffer. Pre-incubation is carried out with compound, enzyme and enzyme stimulator, as for uPA, prior to the incubation with substrate. Incubation time is 60 minutes at performed at 37° C. Data analysis is identical to that described above for uPA, using a known Kₘ for tPA of 250 μM.

Plasmin inhibition is assayed by incubating human plasmin at 0.7 μg/ml with 0.2 mM Chromozym-PL (substrate) and various concentrations of inhibitors in uPA assay buffer. Pre-incubation is carried out as for uPA and the incubation is performed at 37° C. for 30 mins. Data manipulation and percentage inhibition is calculated as for uPA, using a known Kₘ for plasmin of 200 μM.

Analysis

The following Table presents numerical values as to what would constitute an agent that would not work as an I:uPA in accordance with the present invention (i.e. a “fail”) and what would constitute an agent that would work as an I:uPA in accordance with the present invention (i.e. a “pass”). In addition, the following Table presents numerical values as to what would constitute an agent that would work very well as an I:uPA in accordance with the present invention (i.e. a “very good”).

| | | | Selectivity over inhibition of |
|---|---|---|---|
| | Kᵢ for uPA | | tPA and plasmin |
| Pass | <100 nM | AND | >300-fold |
| Fail | >100 nM | OR | <300-fold |
| Very good | <40 nM | AND | >1,000-fold, |
| | | | preferably >1,500-fold, |
| | | | preferably >2,000-fold, |
| | | | preferably >2,500-fold |

MMP Inhibitor

A component of the composition of the present invention maybe an inhibitor of an MMP that has a deleterious effect on wound healing of damaged tissue. Typically, the I:MMP will be capable of being identified as being an I:MMP by an MMP assay—such as the assay protocol presented herein.

Thus, in one aspect, the present invention relates to a method of enhancing the healing of chronic dermal ulcers, including venous stasis ulcers, diabetic ulcers and decubitus ulcers (or pressure sores), by treating the patient with a combination of a selective inhibitor of particular MMPs and a growth factor. This combination therapy is more effective than treatment with the individual agents.

The inhibitors of the adverse MMP can either be applied topically or administered orally, depending on the properties of the inhibitor and the way in which they are formulated.

Thus, according to one aspect of the present invention,the composition may comprise an I:MMP—such as a selective MMP inhibitor—and a growth factor; wherein said MMP has an adverse effect on wound healing in damaged tissue. With the co-administration of these two components a more profound efficacy can be achieved than by administration of either a growth factor or a MMP inhibitor alone. Here, efficacy may be measured by the standard of the FDA in this area, namely the time to closure of chronic dermal ulcers under conditions of best care and compared to best care alone.

In one preferred aspect, topical formulations of selective MMP inhibitors can be co-administered with topically administered growth factors, such as PDGF, either by physically mixing the substances and using a formulation which releases both substances into the damaged tissue, such as the wound, environment, or by applying one substance at a time and using a treatment protocol which separates application of the agents. Alternatively, combined treatment can be achieved using an orally administered MMP inhibitor with topical application of a growth factor.

We believe that the use of certain I:MMP when co-administered with growth factors is very advantageous and was, also, unexpected and unpredictable. In this respect, there are many literature reports show that MMPs are required as part of the cellular response downstream from growth factor receptors. We have determined that, whilst this may be the case, the protective effects of a selective MMP inhibitor on growth factors predominates and this provides the scientific basis for the invention.

In accordance with the present invention, the I:MMP may be applied topically mixed with the growth factor or the I:MMP may be applied topically but at a different time to the growth factor or the I:MMP may be administered orally and the growth factor may be applied topically.

A number of I:MMPs are known.

By way of example, naturally occurring proteinacious inhibitors that exist include Tissue Inhibitors of Metalloproteinases (TIMPs)—see Bode, W., Fernandez-Catalan, C., Grams, F., Gomis-Ruth, F. X., Nagase, H., Tschesche, H., Maskos, K. (1999) Ann. N.Y. Acad. Sci. 878, 73-91 and Vaalamo, M., Leivo, T., Saarialho-Kere, U. (1999) Human Pathology 30 (7), 795-802. These include TIMP-1, TIMP-2, TIMP-3 and TIMP-4.

In addition, synthetic inhibitors of MMP exist. These synthetic inhibitors will typically be organic compounds. Typically the organic compounds will comprise two hydrocarbyl groups linked by a —C(O)N(H)— group. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group, wherein that cyclic group is a polycyclic group, preferably not being a fused polycyclic group.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Preferably the I:MMP inhibits MMP-3 and/or MMP-13. More preferably, the I:MMP is selective vs MMP-1 and/or MMP-2 and/or MMP-9 and/or MMP-14.

Some known MMP inhibitors conform to the following general formula: 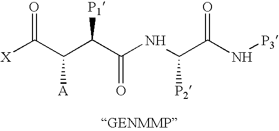
wherein “A” is known as the “alpha” group and XCO is is a zinc-binding group such as a carboxylic acid or hydroxamic acid moiety.

In addition, or in the alternative, a large number of known synthetic inhibitors of MMPs generally conform to one of the generic structures in Scheme presented below, and contain a zinc-binding group (ZBG) which co-ordinates with the catalytic zinc atom of the MMP active site. The ZBG can typically be carboxylic acids, hydroxamic acids, thiols, phosphinates and phosphonates. Reference can be made to recent reviews for examples of these classes (see Whittaker, M.; Floyd, C. D.; Brown, P.; Gearing, A. J. H. Design and Therapeutic Application of Matrix Metalloproteinase Inhibitors. Chem. Rev. 1999, 99, 2735-2776; and Michaelides, M. R.; Curtin, M. L. Recent Advances in Matrix Metalloproteinase Inhibitor Research. Current Pharmaceutical Design, 1999, 5, 787-819).

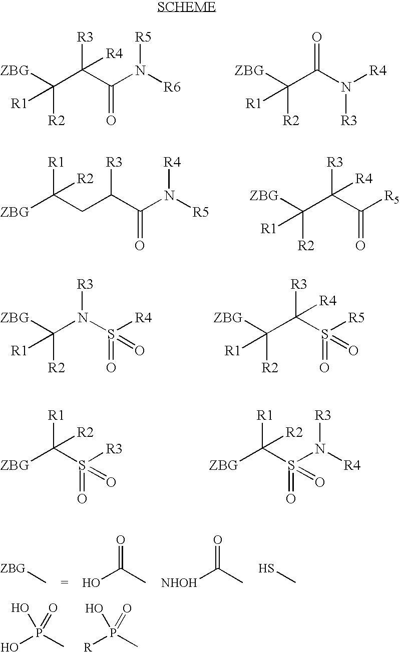

Examples of such suitable I:MMPs are mentioned in WO-A-90/05719, WO-A-99/35124, WO-A-99/29667, WO-A-96/27583, WO-A-99/07675, and WO-A-98/33768. Preferred inhibitors for use in the present invention are described in WO-A-90/05719, WO-A-99/35124, WO-A-99/29667 and PCT/IB00/00667 filed 18 May 2000.

A preferred compound from WO-A-90/05719 is compound 5719—the structural formula for which is presented in the Examples section.

A preferred compound from WO-A-99/29667 is that presented as Example 66 therein (“compound 9470”). The structural formula of Compound 9470 is presented in the Examples section.

A preferred compound from WO-A-99/35124 is that presented as Example 15 therein (“compound 9454”)—the structural formula for which is presented in the Examples section.

Another preferred compound is Example 14 of WO-A-99/35124.

Other preferred compounds are disclosed in PCT/IB00/00667—in particular Example 1, Example 2 and Example 3. A very preferred compound from PCT/IB00/00667 is Example 1.

The inhibitor compounds of WO-A-99/35124 may be presented by the following general formula: 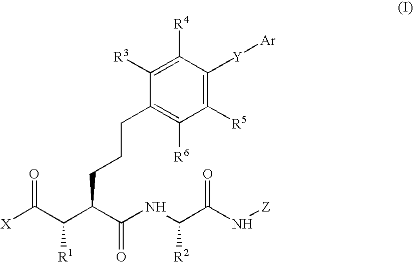
and pharmaceutically acceptable salts thereof, wherein
R¹ is H, OH, C₁₋₄ alkyl, C₁₋₄ alkoxy, or C₂₋₄ alkenyl,
R² is C₁₋₆ alkyl optionally substituted by fluoro, indolyl, imidazolyl, SO₂(C₁₋₄ alkyl), C₅₋₇ cycloalkyl, or by an optionally protected OH, SH, CONH₂, CO₂H, NH₂ or NHC(═NH)NH₂ group, C₅₋₇ cycloalkyl optionally substituted by C₁₋₆ alkyl, or is benzyl optionally substituted by optionally protected OH, C₁₋₆ alkoxy, benzyloxy or benzylthio, wherein the optional protecting groups for said OH, SH, CONH₂, NH₂ and NHC(═NH)NH₂ groups are selected from C₁₋₆ alkyl, benzyl, C₁₋₆ alkanoyl, and where the optional protecting groups for said CO₂H is selected from C₁₋₆ alkyl or benzyl,
R³, R⁵ and R⁶ are each independently selected from H and F,
R⁴ is CH₃, Cl or F,
X is HO or HONH,
Y is a direct link or O,
Z is either a group of formula (a): 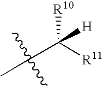
where R¹⁰ is C₁₋₄ alkyl, C₁₋₄ alkoxymethyl, hydroxy(C₂₋₄ alkyl), carboxy(C₁₋₄ alkyl) or (amino or dimethylamino)C₂₋₄ alkyl,
and R¹¹ is phenyl, naphthyl or pyridyl optionally substituted by up to three substituents independently selected from halo and methyl;
or (b) 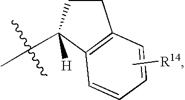
R¹⁴ is H, OH, CH₃ or halo,
Ar is a group of formula (c), (d) or (e): 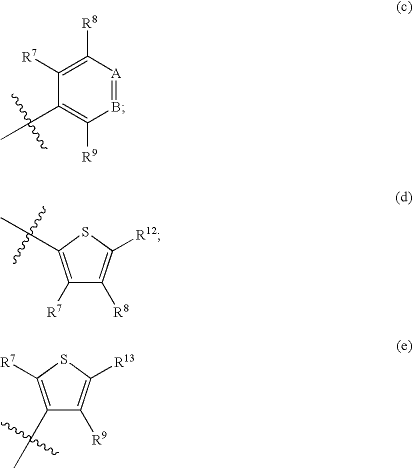
wherein
A is N or CR¹²,
B is N or CR¹³,
provided that A and B are not both N,
R⁷ and R⁹ are each independently H or F,
R⁸, R¹² and R¹³ are each independently H, CN, C₁₋₆ alkyl, hydroxy(C₁₋₆alkyl), hydroxy(C₁₋₆)alkoxy, C₁₋₆ alkoxy(C₁₋₆)alkoxy,(amino or dimethylamino)C₁₋₆ alkyl, CONH₂, OH, halo, C₁₋₆ alkoxy, (C₁₋₆ alkoxy)methyl, piperazinylcarbonyl, piperidinyl, C(NH₂)═NOH or C(═NH)NHOH, with the proviso that at least two of R⁸, R¹² and R¹³ are H.

As indicated preferred compounds from WO-A-99/35124 are Example 15 (hereinafter referred to as “compound 9454”) and Example 14 therein. The formula for Compound 9454 is presented in the Examples section.

Suitable I:MMP compounds for use in the present invention are also disclosed in GB patent application No. 9912961 which was filed on 3 Jun. 1999 (incorporated herein by reference), U.S. patent application Ser. No. 60/169578 filed on 8 Dec. 1999 (incorporated herein by reference) and PCT patent application No. PCT/IB00/00667 filed on 18 May 2000 (incorporated herein by reference). Some relevant teachings of those patent applications are provided herein (see the section titled “PCS10322 Compounds”).

Examples of preferred inhibitors for use in the present invention are shown below.

Inhibitors of MMPs can either be applied topically or administered orally, depending on the properties of the inhibitor and the way in which they are formulated.

| Ex. | Name | Structure | Synthesis |
|---|---|---|---|
| 1 | (3R)-3-({[(1S)-2,2-Dimethyl-1-({[(1R)-1- phenylethyl]amino}carbonyl)propyl]amino}carbonyl)-6-[(3-methyl-4- phenyl)phenyl]hexanoic acid | 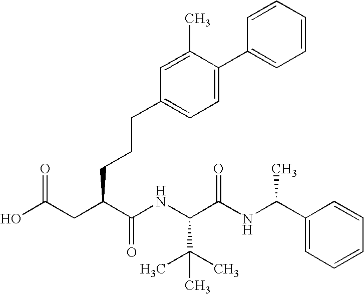 | see Example 1 of WO-A- 99/35124 |
| 2 | N1-[(1S)-2,2-Dimethyl-1-({[(1R)-1- phenylethyl]amino}carbonyl)propyl]- (N4-hydroxy)-(2R)-2-{3-[3-methyl-(4- phenyl)phenyl]propyl}butanediamide | 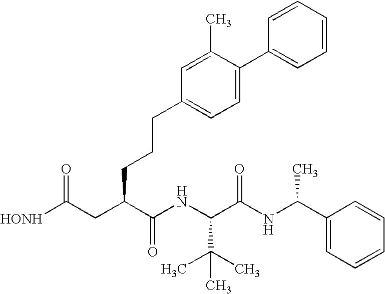 | see Example 3 of WO-A- 99/35124 |
| 3 | (3R)-3-({[(1S)-2,2-Dimethyl-1-({[(1S)- 2-methoxy-1- phenylethyl]amino}carbonyl)- propy]amino}carbonyl)-6-[(3-methyl- 4-phenyl)phenyl]hexanoic acid | 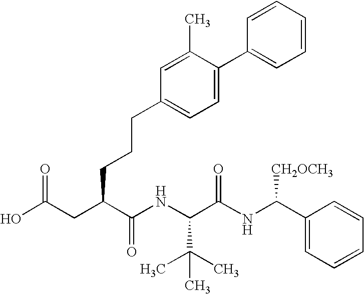 | see Example 14 of WO-A- 99/35124 |
| 4 | (3R)-3-({[(1S)-2,2-Dimethyl-1-({[(1S)- 2-methoxy-1- phenylethyl]amino}carbonyl)propyl]amino}carbonyl)-6-(3′-methoxy-2- methylbiphen-4-yl)hexanoic acid | 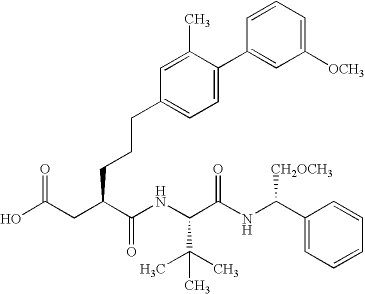 | see Example 15 of WO-A- 99/35124 |
| 5 | (2R)-N1-[(1S)-2,2-Dimethyl-1-({[(1S)- 2-methoxy-1- phenylethyl]amino}carbonyl)propyl]-2- {3-[(3-methyl-4- phenyl)phenyl]propyl}-(N4- hydroxy)butanediamide. | 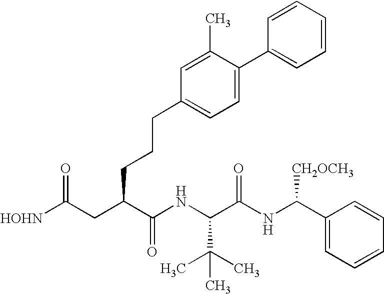 | see Example 16 of WO-A- 99/35124 |
| 6 | N-Hydroxy 2-[(4-{4-[6-(2- hydroxyethoxy)pyridin-2-yl]-3- methylphenyl}piperidin-1- yl)sulphonyl]-2-methylpropanamide | 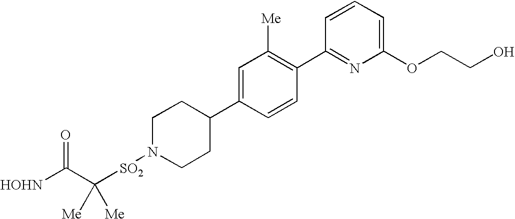 | see Example 1 of PCT/IB00/ 00667 |
| 7 | N-Hydroxy 2-{[4-(4-{6-[2- (methoxy)ethoxy]pyridin-2-yl}-3- methylphenyl)piperidin-1- yl]sulphonyl}-2-methylpropanamide | 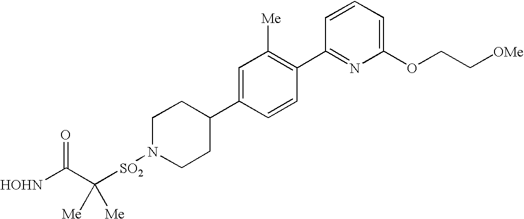 | see Example 2 of PCT/IB00/ 00667 |
| 8 | N-Hydroxy 4-{[4-(4-{6-[2- hydroxyethoxy]pyridin-2-yl}-3- methylphenyl)piperidin-1- yl]sulphonyl}tetrahydro-2H-pyran-4- carboxamide | 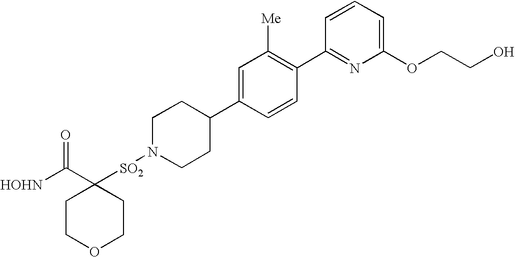 | see Example 3 of PCT/IB00/ 00667 |

(Ex. = Example)

MMP Inhibitor Assay Protocol

The following presents a protocol for identifying one or more agents capable of acting as an I:MMP that would be suitable for use in the composition of the present invention.

Materials

Enzymes

All of the following enzymes were made by standard techniques in the art:
Human MMP-1, catalytic domain, initial stock concentration 1 μM
Human MMP-2, catalytic domain, initial stock concentration 6.94 μM
Human MMP-3, catalytic domain, initial stock concentration 36 μM
Human MMP-9, catalytic domain, initial stock concentration 4.565 μM
Human MMP-14, catalytic domain, initial stock concentration 10 μM.
   Substrates

MMP-1 substrate (Bachem; Cat. No. M-2055) reconstituted in dimethylsulphoxide (DMSO) to give a 1 mM stock and stored frozen (−18° C.). MMP-2, MMP-3, MMP-9 substrate (Neosystem Laboratories; Cat. No. SP970853) reconstituted in DMSO to give a 1 mM stock and stored frozen (−18° C.). MMP-14 substrate (Bachem; Cat. No. M-1895) reconstituted in DMSO to give a 1 mM stock and stored frozen (−18° C.).

Assay Buffers

For MMP-1 the assay buffer used is 50 mM Tris, 200 mM NaCl, 5 mM CaCl₂, 20 μM ZnCl₂, 0.05% (w/v) Brij 35, pH 7.5. For MMP-2, MMP-3 and MMP-9 the assay buffer used is 100 mM Tris, 100 mM NaCl, 10 mM CaCl₂, 0.05% (w/v) Brij 35, pH 7.5. For MMP-14 the buffer used is 50 mM Tris, 100 mM NaCl, 10 mM CaCl₂, 0.25% (w/v) Brij 35, pH 7.5.

Other Materials

APMA (Sigma; Cat. No. A-9563) reconstituted in DMSO to give a 20 mM stock and stored at 4° C. Trypsin (Sigma; T-1426) reconstituted in assay buffer (50 mM Tris, pH 7.5, 100 mM NaCl, 10 mM CaCl₂, 0.25% Brij 35) to give a 0.1 μg/ml stock. Trypsin-chymotrypsin inhibitor, 100 mg/vial (Sigma; T-9777) reconstituted in assay buffer to give a 0.5 μg/ml stock.

Methods

Enzyme Activation

All enzymes are pre-activated at 37° C. with aminophenylmercuric acetate (APMA) or trypsin before being made up to the final concentrations used in the assay. MMP-1 (30 nM) is activated with 0.93 mM APMA for 20 minutes, MMP-2 (30 nM) is activated with 1.32 mM APMA for 1 hour, MMP-3 (1010 nM) is activated with 1.81 mM APMA for 3 hours, MMP-9 (100 nM) is activated with 2 mM APMA for 2 hours and MMP-14 (900 nM) is activated with 0.9 ng/ml trypsin for 25 minutes after which 4.5 ng/ml trypsin inhibitor is added.

MMP Assay Protocol

All assays are carried out in black 96-well plates with a final volume of 100 μl in each well. Compounds are dissolved in dimethylsulphoxide (DMSO) to 1 mM. Solutions are then serially diluted in buffer to give the final concentrations shown. The addition of substrate is preceded by an initial pre-incubation of enzyme and inhibitor at 37° C. for 15 minutes. For MMP-2, MMP-3, MMP-9 and MMP-14 fluorescence is read every 2 minutes at 328 nm γₑₘ and 393nm γₑₓ for 1 hour using a Fluorostar fluorimeter (BMG) with BIOLISE software. For MMP-1 assays the filters used are 355 nm γₑₓ and 440 nm γₑₘ; fluorescence is read every 2 minutes for 1 hour.

Analysis

The following Table presents numerical values as to what would constitute an agent that would not work as an I:MMP3 in accordance with the present invention (i.e. a “fail”) and what would constitute an agent that would work as an I:MMP in accordance with the present invention (i.e. a “pass”). In addition, the following Table presents numerical values as to what would constitute an agent that would work very well as an I:MMP3 in accordance with the present invention (i.e. a “very good”).

| | | | Selectivity over other MMPs |
|---|---|---|---|
| | | | thought to be essential for |
| | | | damaged tissue, such as |
| | Kᵢ for MMP of interest | | wound, healing processes |
| Pass | <100 nM | AND | >100-fold |
| Fail | >100 nM | OR | <100-fold |
| Very good | <40 nM | AND | >200-fold |
| | | | preferably >300-fold, |
| | | | preferably >400-fold, |
| | | | preferably >450-fold |

The above assay protocol may be adapted for other MMP targets.

Other Active Components

The composition of the present invention may also comprise other therapeutic substances in addition to the growth factor and the inhibitor agent.

Antibody

As indicated, the inhibitor agent for use in the composition of the present invention may be one or more antibodies.

The “antibody” as used herein includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and

F(ab′)2 fragments, as well as single chain antibodies (scFv), fusion proteins and other synthetic proteins which comprise the antigen-binding site of the antibody. Furthermore, the antibodies and fragments thereof may be humanised antibodies, for example as described in U.S. Pat. No. 239400. Neutralizing antibodies, i.e., those which inhibit biological activity of the substance polypeptides, are especially preferred for diagnostics and therapeutics.

Antibodies may be produced by standard techniques, such as by immnunisation with the substance of the invention or by using a phage display library.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identifed agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against particular epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal

Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies may also be produced by inducing in viva production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse WD et al (1989) Science 256:1275-128 1).

General Assay Techniques

Any one or more of appropriate targets—such as an amino acid sequence and/or nucleotide sequence for a protein that is upregulated in a damaged tissue, such as a wound, environment—may be used for identifying an agent capable of inhibiting the action of said protein.

The target employed in such a test may be free in solution, affixed to a solid support, borne on a cell surface, or located intracellularly. The abolition of target activity or the formation of binding complexes between the target and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques.

Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

In one preferred aspect, the present invention relates to a method of identifying agents that selectively inhibit one or more protease proteins that are upregulated in a damaged tissue, such as a wound, environment.

Reporters

A wide variety of reporters may be used in the assay methods (as well as screens) of the present invention with preferred reporters providing conveniently detectable signals (eg. by spectroscopy). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3817837; U.S. Pat. No. 3850752; U.S. Pat. No. 3939350; U.S. Pat. No. 3996345; U.S. Pat. No. 4277437; U.S. Pat. No. 4275149 and U.S. Pat. No. 4366241.

Host Cells

The term “host cell”—in relation to the present invention includes any cell that could comprise the target for the agent of the present invention.

Thus, a further embodiment of the present invention provides host cells transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Organism

The term “organism” in relation to the present invention includes any organism that could comprise the target according to the present invention and/or products obtained therefrom. Examples of organisms may include a fungus, yeast or a plant.

The term “transgenic organism” in relation to the present invention includes any organism that comprises the target according to the present invention and/or products obtained.

Transformation of Host Cells/Host Organisms

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et at (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech Mar./Apr. 17-27, 1994). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression of the encoded protein. The protein produced by a recombinant cell may be displayed on the surface of the cell. If desired, and as will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

Therapy

The agents identified by the assay method of the present invention may be used as therapeutic agents—i.e. in therapy applications.

As with the term “treatment”, the term “therapy” includes curative effects, alleviation effects, and prophylactic effects.

The therapy may be on humans or animals.

The therapy can include the treatment of one or more of chronic dermal ulceration, diabetic ulcers, decubitus ulcers (or pressure sores), venous insufficiency ulcers, venous stasis ulcers, burns, corneal ulceration or melts.

The therapy may be for treating conditions associated with impaired damaged tissue, such as wound, healing, where impairment is due to diabetes, age, cancer or its treatment (including radiotherapy), neuropathy, nutritional deficiency or chronic disease.

Pharmaceutical Compositions

The present invention also provides a pharmaceutical composition comprising a therapeutically effective amount of the agent(s) and/or growth factor of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents and/or growth factors of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If the growth factor and/or the inhibitor agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

In a preferred embodiment, the pharmaceutical of the present invention is administered topically.

Hence, preferably the pharmaceutical is in a form that is suitable for topical delivery.

Administration

The term “administered” includes delivery by viral or non-viral techniques. Viral delivery mechanisms include but are not limited to adenoviral vectors, adeno-associated viral (AA V) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In a preferred aspect, the pharmaceutical composition is delivered topically.

Preferably, the composition of the present invention is administered topically for treating chronic dermal ulcers.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA ₂₂₇EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

If the composition is applied topically, then typical doses may be in the order of about 1 to 50 mg/cm² of damaged tissue, such as wound, area.

Formulation

The component(s) of the present invention may be formulated into a pharmaceutical composition, such as by mixing with one or more of a suitable carrier, diluent or excipient, by using techniques that are known in the art.

Pharmaceutically Active Salt

The agent of the present invention may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Animal Test Models

In vivo models may be used to investigate and/or design therapies or therapeutic agents to treat chronic wounds. The models could be used to investigate the effect of various tools/lead compounds on a variety of parameters which are implicated in the development of a treatment of a chronic wound. These animal test models can be used as, or in, the assay of the present invention. The animal test model will be a non-human animal test model.

General Recombinant DNA Methodology Techniques

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc. PCR is described in U.S. Pat. No. 4683195, U.S. Pat. No. 4800195 and U.S. Pat. No. 4965188.

### SUMMARY

In summation, the present invention relates to a pharmaceutical for use in damaged tissue, such as wound, treatment (e.g. healing); the pharmaceutical comprising a composition which comprises: (a) a growth factor; and (b) an inhibitor agent; and optionally (c) a pharmaceutically acceptable carrier, diluent or excipient; wherein the inhibitor agent can inhibit the action of at least one specific protease protein that is upregulated in a damaged tissue, such as a wound, environment.

The present invention also relates to uses of said composition, as well as to process for making same.

Otherwise expressed, the present invention relates to a pharmaceutical for use in damaged tissue, such as wound, treatment (e.g. healing); the pharmaceutical comprising a composition which comprises: (a) a growth factor; and (b) an inhibitor agent; and optionally (c) a pharmaceutically acceptable carrier, diluent or excipient; wherein the inhibitor agent can inhibit the action of at least one specific protease protein that is upregulated in a damaged tissue, such as a wound, environment; and wherein said protease protein would otherwise be capable of detrimentally degrading said growth factor.

### EXAMPLES

The present invention will now be described only by way of example.

Test 1

Biochemical Determination of Protection Growth Factor Degradation by Protease Inhibitors

Experiments are designed to assess the potential of uPA inhibitors and MMP inhibitors to protect growth factors from degradation by individual protease enzymes.

To assess the susceptibility of a growth factor to degradation by a protease, individual growth factors are incubated with a range of protease enzymes (including uPA, tPA, plasmin or MMPs-1, -2, -3, -9, -13 or 14) at 37° C., for times ranging from 15 minutes to 48 hours. The effect of uPA on growth factor degradation is assessed in both the presence and absence of plasminogen.

Degradation of a particular growth factor by individual proteases is then assessed by either quantifying the reduction in growth factor levels or measuring the presence of peptide degradation products.

Biological techniques suitable for the quantification of growth factor degradation include: HPLC detection, Western blots analysis using specific growth factor antibodies and the use of radiolabelled growth factors.

In instances where individual proteases are found to result in measurable growth factor degradation during the incubation period, then protease inhibitor compounds are evaluated for their protective activity against this degradation.

Compounds are pre-incubated (for 15 minutes) and degradation is assessed by one of the methods as described above. All compounds are tested at concentrations previously shown to inhibit the activity of individual proteases as measured against a fluorescent substrate. The vehicle (DMSO) used does not effect growth factor stability.

These experiments demonstrate the potential of I:uPAs (such as those mentioned above) or certain I:MMPs (such as those mentioned above) to protect growth factors from degradation and therefore the clinical potential of treatments involving co-administration with these agents with growth factors.

Test 2

Functional Enhancement of Growth Factor Activity in Cell Biology Experiments

Migration

Experiments are conducted with primary human dermal cells such as fibroblasts, keratinocytes and endothelial cells. Control studies measure the migratory capacity of cells through or over a suitable physiological matrix (e.g. collagen, fibronectin, Matrigel™). Individual growth factors are tested for their ability to enhance the migration of cells over a given time, and the optimum concentration of growth factor is thus determined for future experiments. To assess the effect of individual proteases on cell migration, various concentrations of purified human proteases are pre-incubated with the appropriate growth factor(s). Following this treatment, growth factors are re-tested for their ability to enhance cell migration over this altered matrix. If cell migration is reduced under these circumstances then it was concluded that the protease tested is capable of degrading the matrix over which the cells are migrating. To assess the functional protective effect of protease inhibitors, compounds are added to the matrix prior to addition of the purified protease.

Proliferation

Experiments are conducted with primary human dermal cells such as fibroblasts, keratinocytes and endothelial cells. The endpoint of these studies is cell proliferation as measured by standard methods such as thymidine incorporation or cell number. Individual growth factors are tested for their ability to enhance the proliferation of cells over a given time, and the optimum concentration of growth factor is thus determined for future experiments. Protease inhibitors alone are also tested for their ability to enhance cell proliferation. Combination experiments involve assessing the proliferative effect of growth factors following pre-treatment of the growth factor with a specific protease. To assess the functional protective effect of protease inhibitors, growth factors are pre-incubated with the protease inhibitor compounds prior to addition of the purified protease. Cell proliferation is then determined as described above.

These experiments demonstrate that I:uPAs (such as those mentioned above) and I:MMPs (such as those mentioned above) can protect growth factors and/or growth factor receptors to give an additive and/or synergistic effect on cell function, demonstrating the clinical potential of co-administration of these inhibitors with growth factors.

### Example 1

### The Effect of Human uPA, Plasmin, MMP-3 and MMP-13 and Their Inhibitors on Growth Factors In Vitro

Materials and Methods

Materials

Human recombinant TGF-β2 and KGF-2 were obtained from R&D Systems. Human recombinant VEGF was obtained from Pharmingen. Trypsin, APMA, Trypsin-Chymotrypsin inhibitor, human recombinant PDGF-BB, aprotinin, Tween-20 and goat anti-VEGF antibody, were obtained from Sigma. Antibodies to TGF-β2, KGF-2 and PDGF-BB were obtained from Santa Cruz Biotechnology Inc. Plasmin, human tPA stimulator, S-2288 and S-2444 chromogenic serine and urokinase substrates respectively were obtained from Quadratech. uPA was obtained from Calbiochem. Chromozym-PL was from Boehringer Mannheim. MMP-1, MMP-2, MMP-3, MMP-9, MMP-13 and MMP-14 were cloned, expressed and purified by standard techniques. MMP-13 assay substrate DNP-Pro-Cha-Gly-Cys(Me)-His-Ala-Lys(NMA)NH₂ was obtained from Peptides International Inc. MMP-1 substrate, Dnp-Pro-β-cyclohexyl-Ala-Gly-Cys(Me)-His-Ala-Lys(N-Me-Ala)-NH₂ and MMP-14 substrate, Mca-Pro-Leu-Gly-Leu-Dpa-Ala-Arg-NH₂ were obtained from Bachem. MMP-2, MMP-3, MMP-9 substrate, Mca-Arg-Pro-Lys-Pro-Tyr-Ala-Nva-Trp-Met-Lys(Dnp)-NH₂ was obtained from Neosystem Laboratories. compound 5719, compound 5214, compound 9470 and compound 9454 were synthesised by standard techniques and prepared as a 10 mM stock solutions in DMSO. All electrophoresis and Western blotting reagents were from Invitrogen (NOVEX). Blocking reagent (SuperBlock) was from Pierce and TBS (Tris-buffered saline) was obtained from Bio-Rad. Western blotting development reagents were obtained from Vector Laboratories. All chemicals were reagent grade.

Methods

i) Inhibition of Enzymes by Synthetic Compounds

MMP Assays

Enzyme Activation

All enzymes were pre-activated at 37° C. with aminophenylmercuric acetate (APMA) or trypsin before being made up to the final concentrations used in the assay. MMP-1 (30 nM) was activated with 0.93 mM APMA for 20 minutes, MMP-2 (30 nM) was activated with 1.32 mM APMA for 1 hour, MMP-3 (1010 nM) was activated with 1.81 mM APMA for 3 hours or heat activated at 55° C. for three hours, MMP-9 (100 nM) was activated with 2 mM APMA for 2 hours, human MMP-13 (100 nM) was activated with 2 mM APMA for 2 hours, and MMP-14 (900 nM) was activated with 0.9 ng/ml trypsin for 25 minutes, followed by the addition of 4.5 ng/ml trypsin inhibitor.

Assay Buffers

For MMP-1, the assay buffer used was 50 mM Tris, 200 mM NaCl, 5 mM CaCl₂, 20 μM ZnCl₂, 0.05% (w/v) Brij 35, pH 7.5. For MMP-2, MMP-3 and MMP-9, the assay buffer used was 100 mM Tris, 100 mM NaCl, 10 mM CaCl₂, 0.05% (w/v) Brij 35, pH 7.5. For MMP-13, the assay buffer used was 50 mM Tris, pH 7.5, 200 mM NaCl, 5 mM CaCl₂, 20 mM Zn Cl₂ and 0.02% (w/v) Brij 35. For MMP-14, the assay buffer used was 50 mM Tris, 100 mM NaCl, 10 mM CaCl₂, 0.25% (w/v) Brij 35, pH 7.5.

K₁ Determinations

MMP-1 inhibition was assayed by incubating activated catalytic domain human MMP-1 at 1 nM in assay buffer with 10 μM Dnp-Pro-β-cyclohexyl-Ala-Gly-Cys(Me)-His-Ala-Lys(N-Me-Ala)-NH₂ and six concentrations of inhibitors. The incubation was performed at 37° C. for 60 minutes. The mean velocity between 0 and 60 minutes, which was linear with time, was used to calculate the Kᵢ.

MMP-2, MMP-3 and MMP-9 inhibition was assayed by incubating activated catalytic domain of human MMP-2, MMP-3 and MMP-9 at 1 nM in assay buffer with 5 μM substrate Mca-Arg-Pro-Lys-Pro-Tyr-Ala-Nva-Trp-Met-Lys(Dnp)-NH₂, and six different concentrations of inhibitor. The incubation was performed at 37° C. for 60 minutes. The mean velocity between 0 and 60 minutes, which was linear with time, was then used to calculate the Kᵢ.

MMP-14 inhibition was assayed by incubating activated catalytic domain human MMP-14 at 1 nM in assay buffer with 10 μM Mca-Pro-Leu-Gly-Leu-Dpa-Ala-Arg-NH2 and six concentrations of inhibitor. The incubation was performed at 37° C. for 60 minutes. The mean velocity between 0 and 60 minutes, which was linear with time, was then used to calculate the Kᵢ.

Compound and Substrate Concentrations

The final assay concentrations of inhibitors used in the MMP-1 assays to determine Kᵢ were 50, 40, 30, 20, 10 and 5 μM. For MMP-2, the final assay concentrations of inhibitors used were 1000, 800, 600, 400, 200 and 100 nM. For MMP-3, the final assay concentrations of inhibitors used were 5, 4, 3, 2, 1 and 0.5 nM. For MMP-9 and MMP-14, the final assay concentrations of inhibitors used were 5, 4, 3, 2, 1 and 0.5 μM.

MMP-1, -2, -3, -9 and -14 Assay Protocol

All assays were carried out in a black 96-well plate with a final volume of 100 μl in each well. Inhibitors were dissolved in dimethylsulphoxide (DMSO) to 1 mM. Solutions were then serially diluted in buffer to give the final concentrations shown. The addition of substrate was preceded by an initial pre-incubation of enzyme and inhibitor at 37° C. for 15 minutes. For MMP-2, MMP-3, MMP-9 and MMP-14, fluorescence was read every 2 minutes at 328 nm γₑₘ and 393 nm γₑₘ for 1 hour using a Fluorostar fluorimeter (BMG) with BIOLISE software. For MMP-1 assays, the filters used were 355 nm γₑₓ and 440 nm γₑₘ; fluorescence was read every 2 minutes for 1 hour.

MMP-13 Assays

The IC₅₀ for MMP-13 was determined by incubating activated enzyme at a final concentration of 60 ng/ml (1 nM) in MMP-13 assay buffer, with 10 μM DNP-Pro-Cha-Gly-Cys(Me)-His-Ala-Lys(NMA)NH₂ substrate and varying concentrations of inhibitors (30, 3, 0.3, 0.03, 0.003 and 0.0003 μM) in a final assay volume of 100 μl. Assays were carried out in 96-well microfluor plates. All incubations were performed at 37° C. and fluorescence readings determined at 360 nm γₑₓ and 450 nm γₑₘ.

For the assay, the fluorescence values at time zero were subtracted from those determined at 15 or 20 minutes. The % response was then calculated by comparison to positive controls (enzyme, buffer and substrate in the absence of inhibitor). IC₅₀ values were then determined using FitCurve (Excel Tessella Stats add-in). Outliers were determined using the Grubbs test (Barnet & Lewis, 1994).

Calculation of Kᵢ Values

These were estimated using the following equation:
IC₅₀=(Kᵢ*130 (S/Kₘ),
where S is the substrate concentration and Kₘ the Michaelis-Menton coefficient.
Serine Protease Assays

uPA (urokinase type plasminogen activator) inhibition was assayed by incubating human uPA at 33 IU/ml in 75 mM Tris, pH 8.1, 50 mM NaCl with 180 μM S2444 (substrate) and various concentrations of inhibitors. For the primary screen results, the incubation was performed at 37° C. for 30 minutes. Percentage inhibition was calculated and then plotted against compound concentration using the Excel add-in Fit Curve to give the IC₅₀ and a Kᵢ was calculated from the known Kₘ of the substrate, 90 μM.

tPA (tissue type plasminogen activator) inhibition was assayed by incubating human tPA at 0.4 μg/ml with 0.1 mg/ml tPA stimulator in 75 mM Tris, pH 8.1, 50 mM NaCl with 0.4 mM S2288 (substrate) and various concentrations of inhibitors. The incubation was performed at 37° C. for 60 mins. Percentage inhibition was calculated.

Plasmin inhibition was assayed by incubating human plasmin at 0.7 μg/ml in 75 mM Tris, pH 8.1, 50 mM NaCl with 0.2 mM Chromozym-PL (substrate) and various concentrations of inhibitors. The incubation was performed at 37° C. for 30 mins. Percentage inhibition was calculated.

These assays were carried out in a 96-well plate. The uPA and plasmin assays had a final volume of 200 μl and the tPA assay has a final volume of 100 μl. Inhibitors were dissolved in DMSO to 0.4 mM and then serially diluted to give the final concentrations 100, 30, 10, 3, 1, 0.3, 0.land 0.03 μM. The incubation was performed after an initial pre-incubation at 37° C. for 15 mins and absorbance was read at 405 nM at 0 mins and at the end of the incubation on a SPECTRAMax microplate reader (Molecular Devices Corporation), using SOFTMaxPRO software.

ii) Growth Factor Incubation Conditions

The extent of proteolysis of the growth factors was assayed by incubating TGF-β₂, VEGF, PDGF-BB and KGF-2 with the proteases uPA, plasmin, MMP-3 and MMP-13 in assay buffer (either uPA/plasmin buffer, 50 mM tris-HCl, pH 7.4 or MMP assay buffer, 100 mM Tris, 10 mM NaCl, 10 mM CaCl₂, 0.05% (w/v) Brij 35, pH 7.5). The choice of buffers had no effect on proteolysis during this work. The growth factors were added to the incubation mixture at a final concentration of 7.9 mg/ml, unless otherwise stated.

The effects of uPA were determined by incubation at a typical final concentration of 25 μg/ml (1500 U/ml) with each growth factor. The effects of plasmin were determined at a typical final concentration of 0.1 mg/ml by incubation with the individual growth factors in assay buffer. MMP-3 and -13 were incubated at a typical final concentration of 10 nM with the growth factors in assay buffer. Dual protease assays carried out with uPA and MMP-3 together were performed in 100 mM Tris, 10 nM NaCl, 10 mM CaCl₂, 0.05% (w/v) Brij 35, pH 7.5. All incubations were performed in siliconised tubes (Sigma Aldrich, UK).

The inhibitors used in these experiments were compound 9454, compound 9470 and compound 5214. These were dissolved in DMSO at a concentration of 10 mM. Typical final concentrations for these inhibitors were in the range of 100 μM to 10 nM. Aprotinin was dissolved in the Tris buffer at 10 mg/ml and used at a typical concentration of 10 μg/ml.

All assays were carried out at 37° C. and enzymes were pre-incubated for 15 minutes with or without inhibitor as appropriate, prior to addition of growth factors. After the addition of growth factor, the incubation mixtures were divided into aliquots in siliconised tubes for each time point used. Incubations were carried out over a time course typically of 24 hours, unless otherwise stated. They were stopped by the addition of an equal volume of 2×Novex reducing loading buffer (final concentration 1.09 M glycerol, 141 mM Tris-base, 106 mM Tris-HCl, 73 mM lithium dodecyl sulphate (LDS), 0.5 mM ethylenediaminetetraacetic acid, 0.22 mM Serva Blue G250, 0.175 mM Phenol Red, pH 8.5) and samples prepared for electrophoresis by incubating at 70° C. for 10 minutes.

iii) Electrophoresis

LDS-PAGE was performed using the NOVEX Xcell II Mini-Cell gel apparatus (Groningen, Holland) using a variation on the method of Laemmli (1970). Equal volumes of samples were loaded onto NuPage 4-12% Bis-tris gels with molecular weight markers (SeeBlue Plus2 Pre-stained Standards). Molecular weight determination was performed by comparison of bands with markers of molecular weight 3, 6, 14, 17, 28, 38, 49, 62, 98 and 188 kDa. 79 ng of growth factor was loaded per lane and samples were resolved by vertical slab electrophoresis at 200V for 35 minutes, using running buffer (50 mM 2-(N-morpholino) propane sulphonic acid, 50 mM Tris-base, 3.5mM sodium dodecyl sulphate, 1 mM EDTA, pH 7.3) containing 0.25% NuPAGE Antioxidant in the upper cathodic chamber. Following electrophoresis Western blotting was carried out or gels were stained using SilverXpress kit from NOVEX.

iv) Western Blotting

Samples were separated under reduced and denaturing conditions and electrophoretically transferred to nitrocellulose membranes using the XCell II blot module. Transfer was carried out at 25 V for 60 minutes using NOVEX transfer buffer (20% Methanol, 25 mM bicine, 25 mM Bis-Tris, 1.0 mM EDTA, 0.1% (v/v) antioxidant, pH 7.3). After blotting, membranes were blocked for either 1 or 24 hours using SuperBlock. The membranes were incubated in primary antibody (primary antibodies were at a dilution of 1:400 in TTBS (Tween-20 Tris-buffered saline, 20 mM Tris-HCl, pH 7.4, 500 mM NaCl, 0.1% Tween-20) for one hour. Membranes were then washed and visualisation was performed using the Vector system of peroxidase conjugated secondary antibody; peroxidase was visualised by Nova-Red substrate kit.

v) Quantitation

Analysis of immunoblotted and developed membranes was performed using a GS-700 Imaging Densitometer (Bio-Rad, UK) and SystemOne v4.1.1 software. Inhibitor studies were analysed by quantitation of the loss of parent protein on the blotted membrane over the time course of the experiment. Percentage loss of protein was calculated using the following equations:
D=V_{control}−Vₚₒₛₜ₋ₚᵣₒₜₑₐₛₑ
and
% inhibition=(100−(V_{post-protease plus inhibitor}/D)),
where D is the degradation value and V is the trace volume of parent growth factor band.
Results
1. Calculated Values of Kᵢ for Inhibitors of Plasmin, uPA and tPA

Table 1 gives data showing the potency of compound 5214 as a selective inhibitor of uPA. The results show that compound 5214 is a potent inhibitor of uPA. Full inhibition of tPA and plasmin could not be achieved within the solubility limit of the compound. As IC₅₀ values could not be produced against these enzymes, it was not possible to calculate a Kᵢ against either tPA or plasmin. Hence results show the percentage inhibition of the compound at 100 μM.

By contrast, aprotinin is a selective inhibitor of plasmin: data from the literature as shown in Table 2 to tax support this statement.

Data in Table 3 shows compound 5719 to be a non-selective inhibitor of MMPs, compound 9454 to be a selective MMP-3 inhibitor and compound 9470 to be a selective dual inhibitor of MMP-3 and MMP-13.

2. Growth Factor Proteolysis

Table 4 indicates that proteases are able to digest growth factors that are relevant to wound healing either because the growth factors are endogenously present in normal healing wounds or because they may be added exogenously as pharmaceutical agents to chronic dermal ulcers.

3. Ability of Enzyme Inhibitors to Reduce Growth Factor Degradation

The ability of selective protease inhibitors to reduce the digestion of growth factors by proteases is shown in Tables 5 to 8. (The apparent loss of potency of these compounds compared to experiments where synthetic substrates are used appears to be due to the protein-binding properties of the agents reducing their free concentration within the incubation with growth factors.)

Under appropriate conditions, addition of two inhibitors is able to protect growth factors from degradation more than either of the inhibitors used at the same concentration (Table 9).

TABLE 1
Summary of compound 5214 potency determinations
against uPA, tPA and plasmin

| Protease | Calculated Kᵢ (nM) |
|---|---|
| UPA | 9.6 |
| ″ | 7.8 |
| ″ | 11.0 |
| ″ | 11.0 |
| | Mean ± sem |
| | 9.9 ± 0.76 |
| | % inhibition at 100 μM |
| TPA | 38 |
| ″ | 47 |
| ″ | 50 |
| | Mean ± sem |
| | 45.0 ± 3.61 |
| Plasmin | 31 |
| ″ | 33 |
| ″ | 28 |
| ″ | 27 |
| | Mean ± sem |
| | 29.8 ± 1.38 |

TABLE 2
Summary of Kᵢ values for aprotinin against plasmin, uPA and tPA

| Enzyme | Calculated Kᵢ (nM) | Reference |
|---|---|---|
| Plasmin | 1.0 | Wiman (1980) |
| uPA | 27000 | Lottenberg et al, (1988) |
| tPA | >500000* | Lottenberg et al, (1988) |

*No inhibition seen of tPA by aprotinin at the highest inhibitor concentration of 500 μM.

TABLE 3
Inhibition of MMP-1, -2, -3, -9, -13 and -14 by various synthetic compounds.

| | Calculated Kᵢ(nM) | | | | | |
|---|---|---|---|---|---|---|
| Compound | MMP-1 | MMP-2 | MMP-3 | MMP-9 | MMP-13 | MMP-14 |
| compound 5719 | 0.61 | 0.73 | 0.58 | 0.47 | 1.52 | 3.68 |
| compound 9454 | >19392* | 35215 | 44 | 52396 | 857 | 35481 |
| compound 9470 | 1785 | 269 | 1 | 406 | 0.95 | 1710 |

*limited by compound solubility

TABLE 4
Proteolytic digestion of growth factors by purified proteases*

| | uPA | Plasmin | MMP-3 | MMP-13 |
|---|---|---|---|---|
| PDGF-BB | ++ | +++ | + | (+) |
| TGF-β2 | + | ++ | (+) | + |
| VEGF | ++ | +++ | + | (+) |
| KGF-2 | ++ | +++ | +++ | ++ |

*The extent of hydrolysis is represented by a score from significant (represented as ‘+’) to major (represented as ‘+++’). Reduction of parent growth factor not accompanied by the appearance of degradation products is represented by (+).

TABLE 5
Reduction of uPA-catalysed degradation of
PDGF-BB by compound 5214

| | Percentage inhibition of |
|---|---|
| Inhibitor concentration (μM) | proteolysis* |
| 0.1 | 17 |
| 1 | 76 |
| 10 | 83 |
| 100 | 91 |

*In this case the degradation products at 11.5 kDa were compared

TABLE 6
Reduction of MMP-3-catalysed degradation
of KGF-2 by compound 9454

| | Percentage inhibition of |
|---|---|
| Inhibitor concentration (μM) | proteolysis |
| 0.1 | 45 |
| 1 | 30 |
| 10 | 62 |
| 100 | 68 |

TABLE 7
Reduction of MMP-3-catalysed degradation
of KGF-2 by compound 9470

| | Percentage inhibition of |
|---|---|
| Inhibitor concentration (μM) | proteolysis |
| 0.01 | 24.5 |
| 0.1 | 64.5 |
| 1 | 72.2 |
| 10 | 91.1 |

TABLE 8
Reduction of MMP-13-catalysed degradation
of KGF-2 by compound 9454

| | Percentage inhibition of |
|---|---|
| Inhibitor concentration (μM) | proteolysis |
| 0.1 | 1.60 |
| 1 | 10.0 |
| 10 | 23.7 |
| 100 | 82.9 |

TABLE 9
Inhibition of uPA and MMP-3-mediated KGF-2
degradation by compound 5214 and compound
9454 used either alone or in combination.

| | Percentage inhibition of |
|---|---|
| Inhibitors used (100 μM) | proteolysis |
| compound 5214 | 38.6 |
| compound 9454 | 16.3 |
| compound 5214 and | 49.3 |
| compound 9454 combined | |

References
Barnet, V and Lewis, T. (1994) in Outliers in Statistical Data, p.223, Wiley, Chichester, UK.
Laemmli, U. K. (1970) Nature, 227, 680-685.
Lottenberg, R., Sjak-Shie, N., Fazleabas, A. T. & Roberts, R. M. (1988) Thrombosis Research, 49: 549-556.
Wiman, B. (1980) Thromb. Res. 17, 143-152.

### Example 2

### Non-Selective Protease Inhibitors Perturb Normal Wound Healing in Vivo

Materials and Methods

Test Article and Vehicle

The test article was compound 5719 (0.3% w/v formulation in CMC hydrogel) and the vehicle was CMC hydrogel.

The test article and the vehicle were stored at room temperature in the dark.

Animals

The experiment was performed in 3 female SPF pigs (crossbreed of Danish country, Duroc and Yorkshire). At start of the acclimatisation period the body weight of the animals was about 30 kg.

An acclimatisation period of one week was allowed during which the animals were observed daily in order to reject an animal presenting a poor condition.

Housing

The study took place in an animal room provided with filtered air at a temperature of 21° C.±3° C. and relative humidity of 55%±15%. The room was designed to give 10 air changes per hour. The room was illuminated to give a cycle of 12 hours light and 12 hours darkness. Light was on from 0600 to 1800 h.

The animals were housed individually in pens.

Bedding

The bedding was softwood sawdust “LIGNOCEL 3-4” from Hahn & Co, D-24796 Bredenbek-Kronsburg. Regular analyses for relevant possible contaminants were performed.

Diet

A commercially available pig diet, “Altromin 9033” from Chr. Petersen A/S, DK4100 Ringsted was offered (about 700 g twice daily). Analyses for major nutritive components and relevant possible contaminants were performed regularly.

Drinking Water

Twice daily the animals were offered domestic quality drinking water. Analyses for relevant possible contaminants were performed regularly.

Animal and Pen Identification

The pigs were identified by an eartag with study number and animal number. The pens were identified by a card marked with study number, and animal number.

Surgery

The lesions were established on day 1. The animals were anaesthetised with Stresnil® Vet. Janssen, Belgium (40 mg azaperone/ml, 1 ml/10 kg), and Atropin DAK, Denmark (1 mg atropine/ml, 0.05 ml/kg), given as a single intramuscular injection followed by i.v. injection of Hypnodil® Janssen, Belgium (50 mg metomidate/ml, 1-2 ml).

An area dorso-laterally on either side of the back of the animal were shaved, washed with soap and water, disinfected with 70% ethanol which was rinsed off with sterile saline, and finally dried with sterile gauze.

Eight circular full thickness lesions (diameter 20 mm) were made on the prepared area, four on each side of the spine. The lesions were numbered 1 (most cranial) to 4 (most caudal) on the left side of the animal, and 5 (most cranial) to 8 (most caudal) on the right side of the animal.

Coagulated blood was removed with sterile gauze.

Just before surgery, about 8 hours termination of surgery and whenever necessary thereafter, the animals were given an intramuscular injection of 0.01 mg buprenorphine/kg (Anorfin®, 0.033 ml/kg, A/S GEA, Denmark).

Dosing

After surgery and daily thereafter, the test articles were applied as follows:

| | Animal No. | | | | | |
|---|---|---|---|---|---|---|
| | 1 | | 2 | | 3 | |
| Localisation | Left | Right | Left | Right | Left | Right |
| Cranial | A | B | B | A | A | B |
| | B | A | A | B | B | A |
| | A | B | B | A | A | B |
| Caudal | B | A | A | B | B | A |

A = compound 5719 (0.3% w/v formulation in CMC hydrogel)
B = CMC hydrogel (vehicle)

The dosing volume of each dosing was 1 ml.

Dressing

The dressings were covered with a gauze bandage fixed by Fixomul®. The dressings, the gauze and the Fixomul® were retained by a netlike body-stocking, Bend-a-rete® (Tesval, Italy).

The dressings were changed on a daily basis.

Prior to each changing the animals were anaesthetised with an intramuscular injection in the neck (1.0 ml/10 kg body weight) of a mixture of Zoletil 50® Vet., Virbac, France (125 mg tiletamine and 125 mg zolazepam in 5 ml solvent, 5 ml), Rompun® Vet., Bayer, Germany (20 mg xylazine/ml, 6.5 ml), Ketaminol® Vet., Veterinaria AG, Switzerland (100 mg ketamine/ml, 1.5 ml) and Methadon® DAK, Nycomed DAK, Denmark (10 mg methadon/ml, 2.5 ml).

Observations

Each lesion was observed daily. The outlines of the wound edge and the epithelial edge will be drawn on sterile transparent sheets, and the areas contained inside the edges were measured planimetrically. The measurement of areas was performed by Scan Beam ApS, Nφrregade 10, DK-9560 Hadsund.

Statistics

Data were processed to give group mean values and standard deviations where appropriate. Possible outliers were identified, too. Each variable was tested for normality by the Shapiro-Wilk method. In case of normal distribution, two-way analysis of variance was carried out for the variable with the factor: animal and treatment, and if significant difference were detected, possible intergroup differences were assessed using the least-squares means. Otherwise the possible intergroup differences were identified with Wilcoxon Rank-Sum test.

The statistical analyses were made with SAS® procedures (version 6.12) described in “SAS/STAT® User's Guide, Version 6, Fourth Edition, Vol. 1+2”, 1989, SAS Institute Inc., Cary, N.C. 27513, USA.

Results

| Treatment | Non-epithelialised area | | | | | | | |
|---|---|---|---|---|---|---|---|---|
| | DAY 8 | | | | DAY 9 | | | |
| | MEAN | S.D. | N | p | MEAN | S.D. | N | p |
| compound | 294.0 | 41.1 | 12 | | 248.0 | 23.2 | 12 | |
| 5719 | | | | | | | | |
| CMC hydrogel | 188.0 | 41.7 | 12 | ** | 114.8 | 24.8 | 12 | ** |
| | DAY 10 | | | | DAY 11 | | | |
| | MEAN | S.D. | N | p | MEAN | S.D. | N | p |
| compound | 210.1 | 25.6 | 12 | | 148.9 | 74.5 | 12 | |
| 5719 | | | | | | | | |
| CMC hydrogel | 44.0 | 22.3 | 12 | ** | 13.9 | 10.5 | 12 | ** |

**means p < 0.01
S.D. = standard deviation
N = number of wounds

The Table shows that a non-selective MMP inhibitor perturbs wound healing. Studies using selective MMP inhibitors (in particular MMP-3 inhibitors) showed no effect on normal wound healing.

Similarly for serine proteases, published studies with knock-out mice (Carmeliet et al., 1994) show that in uPA −/− mice, a relatively mild phenotype is apparent, whilst in mice that are uPA −/− and tPA −/−, a more severe phenotype is apparent. The double knock-out, which is the genetic equivalent of using a non-selective serine protease inhibitor, shows increased incidence (in terms of mice and organs affected) and extent of spontaneous fibrin deposition, reduced fertility and life span, and obliterated fibrinolysis. It is therefore reasonable to conclude that a selective inhibitor of uPA will be a far more effective wound healing product than a non-selective agent.

Reference

Carmeliet, P., Schoonjans, L., Kieckens, L., Ream, B., Degen, J., Bronson, R., De Vos, R., van den Oord, J. J., Collen, D. & Mulligan, R. C. (1994) Nature 368:419-424.
   PCS9494 Compounds

As indicated above, suitable inhibitor compounds (agents) for use in the present invention are disclosed in PCT/IB99/01289 (WO-A-00/05214). It is to be understood that if the following teachings refer to further statements of inventions and preferred aspects then those statements and preferred aspects have to be read in conjunction with the aforementioned statements and preferred aspects—viz pharmaceutical compositions either comprising an iUPA and/or an iMMP and a growth factor (as well as the uses thereof) or comprising an iUPA and an iMMP and an optional growth factor (as well as the uses thereof).

The PCS9494 compounds are isoquinolines that are useful as urokinase inhibitors, and are in particular isoquinolinylguanidines useful as urokinase inhibitors. In particular the isoquinolinylguanidine compounds are of the formula (I): 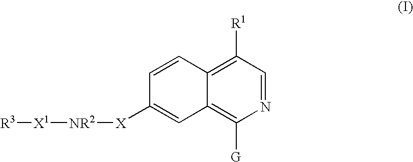
and the pharmaceutically acceptable salts thereof, wherein:
G is N═C(NH₂)₂ or NHC(═NH)NH₂;
R¹ is H or halo;
X is CO, CH₂ or SO₂;
R² is H, aryl, heteroaryl, C₃₋₇ cycloalkyl or C₁₋₄ alkyl each of which C₃₋₇ cycloalkyl and C₁₋₆ alkyl is optionally substituted by one or more substituents independently selected from halo, aryl, het, C₃₋₇ cycloalkyl, C₅₋₇ cycloalkenyl, OH, C₁₋₆ alkoxy, O-het¹, C₁₋₃ alkyl, CO₂R⁷ and NR⁴R⁵;
X¹ is arylene, C₁₋₆ alkylene optionally substituted by one or more R⁶ group, or cyclo(C₄₋₇)alkylene optionally substituted by R⁶, which cyclo(C₄₋₇)alkylene ring can optionally contain a hetero moiety selected from O, S(O)ₚ or NR⁷;
or R² and X¹ can be taken together with the N atom to which they are attached to form an azetidine, pyrrolidine, piperidine or homopiperidine ring;
R³ is CO₂R⁷, CH₂OH, CONR⁸R⁹ or CH₂NR⁸R⁹;
or, when X¹ is taken independently from R² and is methylene optionally substituted by one or more R⁶ group, or is a 1,1-cyclo(C₄₋₇)alkylene optionally containing a hetero moiety selected from O, S(O)ₚ or NR⁷ and optionally substituted by R⁶, then R² and R³ can be taken together with the N and X¹ groups to which they are attached, as a group of formula (IA) or (IB): 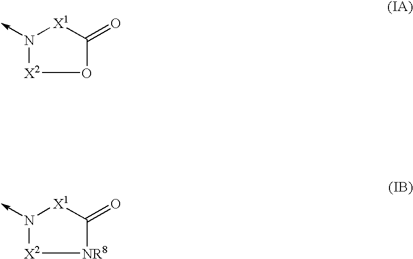
   wherein X² is ethylene, n-propylene or n-butylene;
R⁴ and R⁵ are each independently H, aryl or C₁₋₆ alkyl optionally substituted by aryl;
R⁶ is halo, OH, C₁₋₆ alkoxy, C₁₋₆ alkylthio, C₃₋₇ cycloalkyl, SH, aryl, CO₂R⁷, CONHR⁸, or C₁₋₆ alkyl optionally substituted by aryl, C₁₋₆ alkoxy, CO₂H, OH, CONR⁸R⁹ or by NR⁸R⁹;
R⁷ is H or C₁₋₄ alkyl;
R⁸ and R⁹ are either each independently H, or C₁₋₆ alkyl optionally substituted by OH, CO₂R⁷, C₁₋₆ alkoxy or by NR⁴R⁵; or R⁸ and R⁹ can be taken together with the N atom to which they are attached, to form a 4- to 7-membered ring optionally incorporating an additional hetero-group selected from O, S and NR⁷;
p is 0, 1 or 2;
“aryl” is phenyl optionally substituted by one or more substituents independently selected from C₁₋₆ alkyl, C₁₋₆ alkoxy, or halo;
“het” is a saturated or partly or fully unsaturated 5- to 7-membered heterocycle containing up to 3 hetero-atoms independently selected from O, N and S, and which is optionally substituted by one or more substituents independently selected from C₁₋₆ alkyl, C₁₋₆ alkoxy, CO₂R⁷ or halo;
“heteroaryl” is a fully unsaturated 5- to 7-membered heterocycle containing up to 3 hetero-atoms independently selected from O, N and S, and which is optionally substituted by one or more substituents independently selected from C₁₋₆ alkyl, C₁₋₆ alkoxy, CO₂R⁷ or halo;
“het¹” is tetrahydropyran-2-yl (2-THP);
and “arylene” is phenylene optionally substituted by one or more substituents independently selected from C₁₋₆ alkyl, C₁₋₆ alkoxy, CO₂R⁷ or halo.
“Alkyl” groups can be straight or branched chain. “Halo” in the definitions above refers to F, Cl or Br.
“Cycloalkylene” groups in the definition of the X¹ linker moiety which optionally contains a hetero moiety selected from O, S(O)ₚ or NR⁷ and is optionally substituted by R⁶, can be linked via any available atoms. “1,1-Cycloalkylene” groups in the definition of the X¹ linker moiety which optionally contains a hetero moiety selected from O, S(O)ₚ or NR⁷ and is optionally substituted by R⁶, means the linkage is via a common quaternary centre at one position in the ring, viz. for example: 1,1-cyclobutylene and 4,4-tetrahydropyranylene are to be regarded as both belonging to the same genus of “1,1-cycloalkylene” groups optionally containing a hetero moiety selected from O, S(O)ₚ or NR⁷ and optionally substituted by R⁶.

The two definitions given for the “G” moiety in compounds of formula (I) are of course tautomeric. The skilled man will realise that in certain circumstances one tautomer will prevail, and in other circumstances a mixture of tautomers will be present. It is to be understood that all tautomeric forms of the substances and mixtures thereof are covered.

Preferably G is N═C(NH₂)₂.

Preferably R¹ is halo.

More preferably R¹ is chloro or bromo.

Most preferably R¹ is chloro.

Preferably X is SO₂.

Preferably R² is H, C₃₋₇ cycloalkyl or C₁₋₆ alkyl each of which C₃₋₇ cycloalkyl and C₁₋₆ alkyl is optionally substituted by aryl, het, C₃₋₇ cycloalkyl, OH, Ohet¹, C₁₋₆ alkoxy, CO₂H, CO₂(C₁₋₆ alkyl) or by NR⁴R⁵ , or R² and X¹ can be taken together with the N atom to which they are attached to form an azetidine, pyrrolidine, piperidine or homopiperidine ring.

More preferably R² is H, C₁₋₃ alkyl optionally substituted by aryl or by optionally substituted pyridyl or by NR⁴R⁵ or by HO or by Ohet¹, or R² and X¹ can be taken together with the N atom to which they are attached to form an azetidine, pyrrolidine, piperidine or homopiperidine ring.

Further more preferably R² is H, CH₂CH₂N(CH₃)₂, CH₃, CH₂CH₂OH, CH₂CH₂O(2-THP), pyridinylmethyl, benzyl or methoxybenzyl, or R² and X¹ can be taken together with the N atom to which they are attached to form an azetidine, pyrrolidine, piperidine or homopiperidine ring linked to the R³ moiety via the 2-position of said ring.

Most preferably R² is H, CH₂CH₂N(CH₃)₂, CH₃, CH₂CH₂OH, CH₂CH₂O(2-THP) or R² and X¹ are taken together with the N atom to which they are attached to form a pyrrolidine ring linked to the R³ moiety via the 2-position.

Preferably X¹ is phenylene optionally substituted by one or two substituents independently selected from methoxy and halo, or is C₁₋₃ alkylene optionally substituted by one or more group selected from aryl or (C₁₋₆ alkyl optionally substituted by aryl, C₁₋₆ alkoxy, CO₂H, OH, NH₂ or CONH₂), or is cyclo(C₄₋₇)alkylene optionally contain a hetero moiety selected from O or NR₇, which ring is optionally substituted by R⁶, or is taken together with R² and with the N atom to which they are attached to form an azetidine, pyrrolidine, piperidine or homopiperidine ring.

More preferably, X¹ is methylene optionally substituted by one or more group selected from aryl or (C₁₋₄ alkyl optionally substituted by OH, NH₂ or CONH₂),
or is cyclobutylene, cyclopentylene, cyclohexylene, cycloheptylene, tetrahydropyranylene, piperidinylene substituted by R⁷,
or is taken together with R² and with the N atom to which they are attached to form an azetidine, pyrrolidine, piperidine or homopiperidine ring.

Yet more preferably X¹ is C(CH₃)₂, 1,1-cyclopentylene, 4,4-tetrahydropyranylene, N-methyl-4,4-piperidinylene, CH₂, CH(CH(CH₃)₂), CH(CH₂)₄NH₂, CH(CH₂)₃NH₂, CH(CH₂)CONH₂, 1,1-cyclobutylene, 1,1-cyclopentylene, 1,1-cyclohexylene, 1,1-cycloheptylene, N-methyl4,4-piperidinylene, 4,4-tetrahydropyranylene, or is taken together with R² and with the N atom to which they are attached to form an azetidine, pyrrolidine, piperidine or homopiperidine ring linked to the R³ moiety via the 2-position.

Most preferably X¹ is C(CH₃)₂, 1,1-cyclopentylene, 4,4-tetrahydropyranylene, N-methyl-4,4-piperidinylene, or is taken together with R² and with the N atom to which they are attached to form an azetidine, pyrrolidine, piperidine ring linked to the R³ moiety via the 2-position.

Preferably R³ is CO₂R⁷ or CONR⁸R⁹.

More preferably R³ is CO₂H, CONH₂, CON(CH₃)(CH₂)₂OH, CON(CH₃)(CH₂)₂NHCH₃, CO₂(C₁₋₃alkyl, CONH(CH₂)₂OH, CONH(CH₂)₂OCH₃, (morpholino)CO or (4-methylpiperazino)CO.

Most preferably R³ is CO₂H.

A preferred group of substances (a) are the compounds where X is SO₂ in which the R³—X¹—NR²— moiety is, where X¹ is taken independently from R² and is methylene optionally substituted by one or more R⁶ group, or is a 1,1-cyclo(C₄₋₇)alkylene optionally containing a hetero moiety selected from O, S(O)ₚ or NR⁷ and optionally substituted by R⁶,
and R² and R³ can be taken together with the N and X¹ groups to which they are attached, as a group of formula (IA) or (IB): 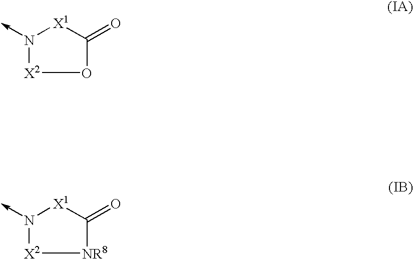
   wherein X² is ethylene, n-propylene or n-butylene.

In this group of substances (a) X¹ is preferably C(CH₃)₂, 1,1-cyclobutylene, 1,1-cyclopentylene, 1,1-cyclohexylene, 4,4-tetrahydropyranylene or N-methyl4,4-piperidinylene, most preferably 1,1-cyclopentylene.

In this group of substances (a) X² is preferably ethylene.

A preferred group of substances are the compounds in which the substituent R¹ has the values as described by the Examples below, and the salts thereof.

A preferred group of substances are the compounds in which the substituent X has the values as described by the Examples below, and the salts thereof.

A preferred group of substances are the compounds in which the substituent R² has the values as described by the Examples below, and the salts thereof.

A preferred group of substances are the compounds in which the substituent X¹ has the values as described by the Examples below, and the salts thereof.

A preferred group of substances are the compounds in which the substituent R³ has the values as described by the Examples below, and the salts thereof.

Another preferred group of substances are the compounds in which each of the substituents R¹, X, R² X¹ and R³ have the values as described by the Examples below, and the salts thereof.

A preferred group of substances are the compounds where R¹ is chloro or bromo; X is SO₂;
R² is H, CH₂CH₂N(CH₃)₂, CH₃, CH₂CH₂OH, CH₂CH₂O(2-THP), pyridinylmethyl, benzyl or methoxybenzyl, or R² and X¹ can be taken together with the N atom to which they are attached to form an azetidine, pyrrolidine, piperidine or homopiperidine ring linked to the R³ moiety via the 2-position of said ring;
X¹ is C(CH₃)₂, 1,1-cyclopentylene, 4,4-tetrahydropyranylene, N-methyl-4,4-piperidinylene, CH₂, CH(CH(CH₃)₂), CH(CH₂)₄NH₂, CH(CH₂)₃NH₂, CH(CH₂)CONH₂, 1,1-cyclobutylene, 1,1-cyclopentylene, 1,1-cyclohexylene, 1,1-cycloheptylene, N-methyl-4,4-piperidinylene, 4,4-tetrahydropyranylene, or is taken together with R² and with the N atom to which they are attached to form an azetidine, pyrrolidine, piperidine or homopiperidine ring linked to the R³ moiety via the 2-position;
R³ is CO₂H, CONH₂, CON(CH₃)(CH₂)₂OH, CON(CH₃)(CH₂)₂NHCH₃, CO₂(C₁₋₃alkyl), CONH(CH₂)₂OH, CONH(CH₂)₂OCH₃, (morpholino)CO or (4-methylpiperazino)CO;
and the salts thereof

Another preferred group of substances are those in which R¹ is chloro; X is SO₂;
R² is H, CH₂CH₂N(CH₃)₂, CH₃, CH₂CH₂OH, CH₂CH₂O(2-THP) or R² and X¹ are taken together with the N atom to which they are attached to form a pyrrolidine ring linked to the R³ moiety via the 2-position;
X¹ is C(CH₃)₂, 1,1-cyclopentylene, 4,4-tetrahydropyranylene, N-methyl-4,4-piperidinylene, or is taken together with R² and with the N atom to which they are attached to form an azetidine, pyrrolidine, piperidine ring linked to the R³ moiety via the 2-position;
and R³ is CO₂H;
and the salts thereof.

Another preferred group of substances are the compounds of the Examples below and the salts thereof. More preferred within this group are the compounds of Examples 32(b), 34(b), 36(b), 37(b), 38, 39(a and b), 41(b), 43(b), 44(b), 71, 75, 76, 78, 79, 84(b), and 87(b and c) and the salts thereof.

Preferred compounds or salts are selected from:
N-[(4-chloro-1-guanidino-7-isoquinolinyl)sulphonyl]-D-proline;
2-{[(4-chloro-1-guanidino-7-isoquinolinyl)sulphonyl]amino}isobutyric acid;
1-{[(4-chloro-1-guanidino-7-isoquinolinyl)sulphonyl]amino}cyclobutanecarboxylic acid;
N-[(4-chloro-1-guanidino-7-isoquinolinyl)sulphonyl]cycloleucine;
N-[(4-chloro-1-guanidino-7-isoquinolinyl)sulphonyl]cycloleucine;
1-{[(4-chloro-1-guanidino-7-isoquinolinyl)sulphonyl]amino)-N-(2-hydroxyethyl)cyclopentanecarboxamine;
1-{[(4-chloro-1-guanidino-7-isoquinolinyl)sulphonyl]amino)-N-[2-(dimethylamino)ethyl]cyclopentanecarboxamine;
1-{[(4-chloro-1-guanidino-7-isoquinolinyl)sulphonyl]amino}-N-[2-(dimethylamino)ethyl]cyclopentanecarboxamine;
N-[(4-chloro-1-guanidino-7-isoquinolinyl)sulphonyl]-N-[2-(dimethylamino)ethyl]cycloleucine;
1-{[(4-chloro-1-guanidino-7-isoquinolinyl)sulphonyl]amino)cyclohexanecarboxylic acid;
4-{[(4chloro-1-guanidino7-isoquinolinyl)sulphonyl]amino}tetrahydro-2H-pyran-4carboxylic acid;
tert-butyl (2R)1-({4-chloro-1-guanidino-7-isoquinolinyl}sulphonyl)-2-piperidinecarboxylate;
(2R)-1-({4-chloro-1-guanidino-7-isoquinolinyl}sulphonyl)-2-piperidinecarboxylic acid;
1-[({4-chloro-1-guanidino-7-isoquinolinyl}sulphonyl)amino]-N-(2-hydroxyethyl)-N-methylcyclopentanecarboxamide;
1-[({4-chloro-1-guanidino-7-isoquinolinyl}sulphonyl)amino]-N-(2-methoxyethyl)cyclopentanecarboxamide;
4-chloro-1-guanidino-N-[1-(morpholinocarbonyl)cyclopentyl]-7-isoquinolinesulphonamide;
4-chloro-1-guanidino-N-{1-[(4-methylpiperazino)carbonyl]cyclopentyl}-7-isoquinolinesulphonamide;
N-({4-bromo-1-guanidino-7-isoquinolinyl}sulphonyl)-N-[2-(dimethylamino)ethyl]cycloleucine;
1-{[(4-chloro-1-guanidino-7-isoquinolinyl)sulphonyl][2-(tetrahydro-2H-pyran-2-yloxy)ethyl]amino}cyclopentanecarboxylic acid; and
N″-{4-chloro-7-[(10-oxo-9-oxa-6-azaspiro[4.5]dec-6-yl)sulphonyl]-1-isoquinolinyl}guanidine;
and the pharmaceutically acceptable salts thereof.

The invention further provides Methods for the production of substances of the invention, which are described below and in the Examples. The skilled man will appreciate that the substances of the invention could be made by methods other than those herein described, by adaptation of the methods herein described in the sections below and/or adaptation thereof, and of methods known in the art.

In the Methods below, unless otherwise specified, the substituents are as defined above with reference to the compounds of formula (I) above.

Method 1

Compounds of formula (I) can be obtained from the corresponding 1-aminoisoquinoline derivative (II): 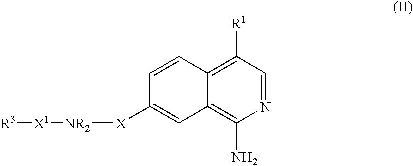
by reaction with cyanamide (NH₂CN) or a reagent which acts as a “NHC⁺═NH” synthon such as carboxamidine derivatives, e.g. 1H-pyrazole-1-carboxamidine (M. S. Bernatowicz, Y. Wu, G. R. Matsueda, J. Org. Chem., 1992, 57, 2497), the 3,5-dimethylpyrazole analogue thereof (M. A. Brimble et al, J.Chem.Soc.Perkin Trans.I (1990)311), simple O-alkylthiouronium salts or S-alkylisothiouronium salts such as O-methylisothiourea (F. El-Fehail et al, J.Med.Chem.(1986), 29, 984), S-methylisothrouronium sulphate (S. Botros et al; J.Med.Chem.(1986)29,874; P. S. Chauhan et al, Ind. J. Chem., 1993, 32B, 858) or S-ethylisothiouronium bromide (M. L. Pedersen et al, J.Org.Chem.(1993) 58, 6966). Alternatively aminoiminomethanesulphinic acid, or aminoiminomethanesulphonic acid may be used (A. E. Miller et al, Synthesis (1986) 777; K. Kim et al, Tet.Lett.(1988) 29,3183).

Other methods for this transformation are known to those skilled in the art (see for example, “Comprehensive Organic Functional Group Transformations”, 1995, Pergamon Press, Vol 6 p639, T. L. Gilchrist (Ed.); Patai's “Chemistry of Functional Groups”, Vol. 2. “The Chemistry of Amidines and Imidates”, 1991, 488).

Aminoisoquinolines (II) may be prepared by standard published methods (see for example, “The Chemistry of Heterocyclic Compounds” Vol. 38 Pt. 2 John Wiley & Sons, Ed. F. G. Kathawala, G. M. Coppolq, H. F. Schuster) including, for example, by rearrangement from the corresponding carboxy-derivative (Hoffmann, Curtius, Lossen, Schmidt-type rearrangements) and subsequent deprotection.

Aminoisoquinolines (II) may alternatively be prepared by direct displacement of a leaving group such as Cl or Br with a nitrogen nucleophile such as azide (followed by reduction), or by ammonia, or through Pd-catalysis with a suitable protected amine (such as benzylamine) followed by deprotection using standard conditions well-known in the art.

Haloisoquinolines are commercially available or can alternatively be prepared by various methods, for example those described in: Goldschmidt, Chem.Ber.(1895)28,1532; Brown and Plasz, J.Het.Chem.(1971)6,303; U.S. Pat. No. 3,930,837; Hall et al, Can.J.Chem.(1966)44,2473; White, J.Org.Chem.(1967)32,2689; and Ban, Chem.Pharm.Bull.(1964)12,1296.

1,4-(Dichloro- or dibromo)isoquinolines can be prepared by the method described by M. Robison et al in J.Org.Chem.(1958)23,1071, by reaction of the corresponding isocarbostyryl compound with PCl₅ or PBr₅.

Method 2

Compounds of formula (I) can be obtained from the corresponding aminoisoquinoline derivative (II) as defined in Method 1 above, via reaction with a reagent which acts as a protected amidine(2+) synthon (III), 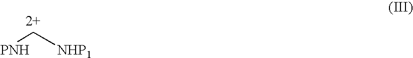
such as a compound PNHC(═X)NHP₁, PN═CXNHP₁ or PNHCX═NP₁, where X is a leaving group such as Cl, Br, I, mesylate, tosylate, alkyloxy, etc., and where P and P₁ may be the same or different and are N-protecting groups such as are well-known in the art, such as t-butoxycarbonyl, benzyloxycarbonyl, arylsulphonyl such as toluenesulphonyl, nitro, etc.

Examples of reagents that act as synthons (III) include N,N′-protected-S-alkylthiouronium derivatives such as N,N′-bis(t-butoxycarbonyl)-S-Me-isothiourea, N,N′-bis(benzyloxycarbonyl)-S-methylisothiourea, or sulphonic acid derivatives of these (J. Org. Chem. 1986, 51, 1882), or S-arylthiouronium derivatives such as N,N′-bis(t-butoxycarbonyl)-S-(2,4-dinitrobenzene) (S. G. Lammin, B. L. Pedgrift, A. J. Ratcliffe, Tet. Lett. 1996, 37, 6815), or mono-protected analogues such as [(4-methoxy-2,3,6-trimethylphenyl)sulphonyl]-carbamimidothioic acid methyl ester or the corresponding 2,2,5,7,8-pentamethylchroman-6-sulphonyl analogue (D. R Kent, W. L. Cody, A. M. Doherty, Tet. Lett., 1996, 37, 8711), or S-methyl-N-nitroisothiourea (L. Fishbein et al, J.Am.Chem.Soc. (1954) 76, 1877) or various substituted thioureas such as N,N′-bis(t-butoxycarbonyl)thiourea (C. Levallet, J. Lerpiniere, S. Y. Ko, Tet. 1997, 53, 5291) with or without the presence of a promoter such as a Mukaiyama's reagent (Yong, Y. F.; Kowalski, J. A.; Lipton, M. A. J. Org. Chem., 1997, 62, 1540), or copper, mercury or silver salts, particularly with mercury (II) chloride. Suitably N-protected O-alkylisoureas may also be used such as O-methyl-N-nitroisourea (N. Heyboer et al, Rec.Chim.Trav.Pays-Bas (1962)81,69). Alternatively other guanylation agents known to those skilled in the art such as 1-H-pyrazole-1-[N,N′-bis(t-butoxycarbonyl)]carboxamidine, the corresponding bis-Cbz derivative (M. S. Bernatowicz, Y. Wu, G. R. Matsueda, Tet. Lett. 1993, 34, 3389) or monoBoc or mono-Cbz derivatives may be used (B. Drake. Synthesis, 1994, 579, M. S. Bernatowicz. Tet. Lett. 1993, 34, 3389). Similarly, 3,5-dimethyl-1-nitroguanylpyrazole may be used (T. Wakayima et al, Tet.Lett.(1986)29,2143).

The reaction can conveniently be carried out using a suitable solvent such as dichloromethane, N,N-dimethylformamide (DMF), methanol.

The reaction is also conveniently carried out by adding mercury (II) chloride to a mixture of the aminoisoquinoline (II) and a thiourea derivative of type (III) in a suitable base/solvent mixture such as triethylamine/dichloromethane.

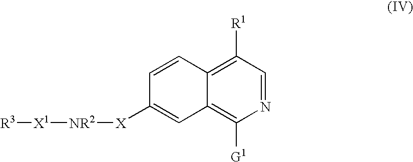

The product of this reaction is the protected isoquinolinylguanidine (IV), where G¹ is a protected guanidine moiety N═C(NHP)(NHP₁) or tautomer thereof, where P and P₁ are nitrogen-protecting groups such as t-butoxycarbonyl (“Boc”), benzyl, benzyloxycarbonyl, etc., which can conveniently be deprotected to give (I) or a salt thereof.

For example, if the protecting group P and/or P₁ is t-butoxycarbonyl, conveniently the deprotection is carried out using an acid such as trifluoroacetic acid (TFA) or hydrochloric acid, in a suitable solvent such as dichloromethane, to give the bistrifluoroacetate salt of (I).

If P and/or P₁ is a hydrogenolysable group, such as benzyloxycarbonyl, the deprotection could be performed by hydrogenolysis.

Other protection/deprotection regimes include: nitro (K. Suzuki et al, Chem.Pharm.Bull. (1985)33,1528, Nencioni et al, J.Med.Chem.(1991)34,3373, B. T. Golding et al, J.C.S.Chem.Comm.(1994)2613; p-toluenesulphonyl (J. F. Callaghan et al, Tetrahedron (1993) 49 3479; mesitylsulphonyl (Shiori et al, Chem.Pharm.Bull.(1987)35,2698, ibid.(1987)35,2561, ibid., (1989)37,3432, ibid., (1987)35,3880, ibid., (1987)35,1076; 2-adamantoyloxycarbonyl (Iuchi et al, ibid., (1987) 35, 4307; and methylsulphonylethoxycarbonyl (Filippov et al, Syn.Lett.(1994)922)

It will be apparent to those skilled in the art that other protection and subsequent deprotection regimes during synthesis of a compound of the invention may be achieved by various other conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

Method 3

Compounds of the formula (I) can be obtained from compounds of formula (V) 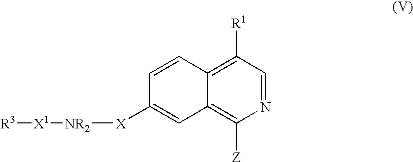
where Z is a suitable leaving group such as Cl, Br or OPh, by displacement of the leaving group by the free base of guanidine.

Compounds of formula (V) are available as mentioned above in the section on preparation of compounds of formula (II) in Method 1, and routine variation thereof.

The free base of guanidine may conveniently be generated in situ from a suitable salt, such as the hydrochloride, carbonate, nitrate, or sulphate with a suitable base such as sodium hydride, potassium hydride, or another alkali metal base, preferably in a dry non-protic solvent such as tetrahydrofuran (THF), DMSO, N,N-dimethylformamide (DMF), ethylene glycol dimethyl ether (DME), N,N-dimethyl acetamide (DMA), toluene or mixtures thereof. Alternatively it can be generated from a suitable salt using an alkoxide in an alcohol solvent such as potassium t-butoxide in t-butanol, or in a non-protic solvent as above.

The thus formed free guanidine can be combined with the 1-isoquinoline derivative (V), and the reaction to form compounds of formula (I) can be carried out at from room temperature to 200° C., preferably from about 50° C. to 150° C., preferably for between 4 hours and 6 days.

It will be clear to those skilled in the art, that some of the functionality in the R³, R² and/or X¹ groups may need to be either protected and released subsequent to guanylation or added, or generated after the guanidine moiety had been added to the substrate.

For example, an acid group could be carried through the guanylation stage while protected as an ester and subsequently hydrolyseded. Base-catalysed hydrolysis of an ethyl ester and acid-catalysed hydrolysis of a t-butyl ester are two such suitable examples of this. In another example, an alcohol may be protected with groups well documented in the literature such as a 2-tetrahydropyranyl ether (2-THP) and subsequently removed by treatment with acid.

The addition of new functionality after the guanidine moiety has been installed is also encompassed by the invention. For example, alkylation of the sulphonamido NH (i.e. “X—NR²” is SO₂NH) with an alkyl halide may be performed in the presence of a base such as potassium carbonate and optionally in the presence of a promoter such as KI. In another example, an acid group may be converted to an amide through a range of coupling conditions known to those skilled in the art, or conveniently though the acid chloride while in the presence of a free or protected guanidine. Alternatively an ester group can be directly reacted with an amine to generate an amide; if this occurs in an intramolecular process, a lactam may be formed. Using similar methodology esters and lactones may be prepared. Additional functionality could have been present in a protected form at this stage and subsequently revealed—such as an amino group which could be protected by groups well documented in the literature, e.g. a Boc group and subsequently removed under standard conditions, such as treatment with a strong base such as HCl or TFA.

Method 4

Compounds of the invention where one or more substituent is or contains a carboxylic acid group or carbamoyl group can be made from the corresponding compound where the corresponding substituent is a nitrile by full or partial hydrolysis. Compounds of the invention where one or more substituent is or contains a carboxylic acid group can be made from the corresponding compound where the corresponding substituent is a carbamoyl moiety, by hydrolysis.

The hydrolysis can be carried out by methods well-known in the art, for example those mentioned in “Advanced Organic Chemistry” by J. March, 3rd edition (Wiley-Interscience) chapter 6-5, and references therein. Conveniently the hydrolysis is carried out using concentrated hydrochloric acid, at elevated temperatures, and the product forms the hydrochloride salt.

Method 5

Where desired or necessary the compound of formula (I) is converted into a pharmaceutically acceptable salt thereof. A pharmaceutically acceptable salt of a compound of formula (I) may be conveniently be prepared by mixing together solutions of a compound of formula (I) and the desired acid or base, as appropriate. The salt may be precipitated from solution and collected by filtration, or may be collected by other means such as by evaporation of the solvent.

Other Methods

Compounds of the formula (I) where one or more substituent is or contains Cl or Br may be dehalogenated to give the corresponding hydrido compounds of formula (I) by hydrogenolysis, suitably using a palladium on charcoal catalyst, in a suitable solvent such as ethanol at about 20° C. and at elevated pressure.

Compounds of formula (I) where one or more substituent is or contains a carboxy group may be prepared from a compound with a group hydrolysable to give a carboxy moiety, for example a corresponding nitrile or ester, by hydrolysis, for example by acidic hydrolysis with e.g. conc. aq. HCl at reflux. Other hydrolysis methods are well known in the art.

Compounds of formula (I) in which one or more substituent is or contains an amide moiety may be made via reaction of an optionally protected corresponding carboxy compund, either by direct coupling with the amine of choice, or via initial formation of the corresponding acid chloride or mixed anhydride, and subsequent reaction with the amine, followed by deprotection if appropriate. Such transformations are well-known in the art.

Certain of the compounds of formula (I) which have an electrophilic group attached to an aromatic ring can be made by reaction of the corresponding hydrido compound with an electrophilic reagent.

For example sulphonylation of the aromatic ring using standard reagents and methods, such as fuming sulphuric acid, gives a corresponding sulphonic acid. This can then be optionally converted into the corresponding sulphonamide by methods known in the art, for example by firstly converting to the acid chloride followed by reaction with an amine.

Certain of the compounds of the invention can be made by cross-coupling techniques such as by reaction of a compound containing a bromo-substituent attached to e.g. an aromatic ring, with e.g. a boronic acid derivative, an olefin or a tin derivative by methods well-known in the art, for example by the methods described in certain of the Preparations below.

Certain of the compounds of the invention having an electrophilic substituent can be made via halogen/metal exchange followed be reaction with an electrophilic reagent For example a bromo-substituent may react with a lithiating reagent such as n-butyllithium and subsequently an electrophilic reagent such as CO₂, an aldehyde or ketone, to give respectively an acid or an alcohol.

Compounds of the invention are available by either the methods described herein in the Methods and Examples or suitable adaptation thereof using methods known in the art. It is to be understood that the synthetic transformation methods mentioned herein may be carried out in various different sequences in order that the desired compounds can be efficiently assembled. The skilled chemist will exercise his judgement and skill as to the most efficient sequence of reactions for synthesis of a given target compound.

Experimental Section

General Details

Melting points (mp) were determined using either Gallenkamp or Electrothermal melting point apparatus and are uncorrected. Proton nuclear magnetic resonance (¹H NMR) data were obtained using a Varian Unity 300 or a Varian Inova 400. Low resolution mass spectral (LRMS) data were recorded on a Fisons Instruments Trio 1000 (thermaspray) or a Finnigan Mat. TSQ 7000 (APCI). Elemental combustion analyses (Anal.) were determined by Exeter Analytical UK. Ltd.

Column chromatography was performed using Merck silica gel 60 (0.040-0.063 mm). Reverse phase column chromatography was performed using Mitsubishi MCl gel (CHP 20P).

The following abbreviations were used: ammonia solution sp. gr. 0.880 (0.880NH₃); diethyl azodicarboxylate (DEAD); 1,2-dimethoxyethane (DME); N,N-dimethylacetamide (DMA); N,N-dimethylformamide (DMF); dimethylsulphoxide (DMSO); tetrahydrofuran (THF); trifluoroacetic acid (TFA); toluene (PhMe); methanol (MeOH); ethyl acetate (EtOAc) propanol (PrOH). Other abbreviations are used according to standard chemical practice.

Some nomenclature has been allocated using the IUPAC NamePro software available from Advanced Chemical Development Inc. It was noted following some preparations involving guanylation of intermediates containing a quaternary centre adjacent to a base-sensitive group e.g. an ester, that some racemisation had occurred, so in such cases there may be a mixture of enantiomers produced.

### EXAMPLES

### Example 1

### (a) tert-Butyl 2-{[(4-chloro-1-guanidino-7-isoquinolinyl)sulphonyl]amino}benzoate

### (b) 2-{[(4-Chloro-1-guanidino-7-isoquinolinyl)sulphonyl]amino}benzoic acid hydrochloride

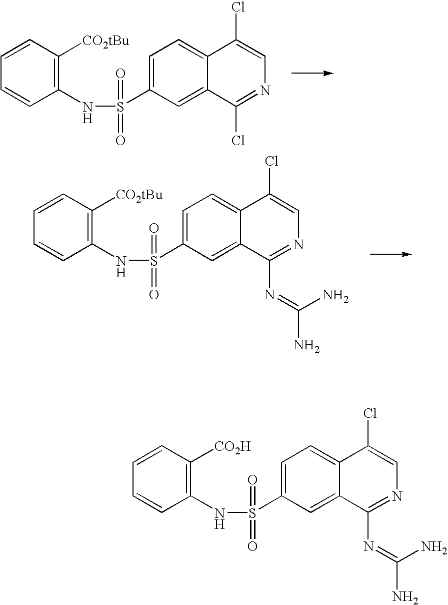

Guanidine hydrochloride (60 mg, 0.63 mmol) was added in one portion to a suspension of NaH (18 mg, 80% dispersion by wt in mineral oil, 0.6 mmol) in DMSO (3.0 mL) and the mixture was heated at 60° C. under N₂ for 30 min. tert-Butyl 2-{[(1,4-dichloro-7-isoquinolinyl)sulphonyl]amino}benzoate (110 mg, 0.24 mmol) was added and the mixture heated at 100° C. for 24 h. The cooled mixture was poured into water and extracted with EtOAc (×3) and the combined organic phase was then washed with brine and evaporated in vacuo. The residue was purified by column chromatography upon silica gel using CH₂Cl₂-MeOH-0.880NH₃ (97:3:0.3 to 95:5:0.5) as eluant to give a yellow resin (36 mg). This resin was suspended in water and extracted with ether (×3). The combined organic phase was washed with brine and evaporated in vacuo to give tert-butyl 2-{[(4-chloro-1-guanidino-7-isoquinolinyl)sulphonyl]amino}benzoate (30 mg, 0.063 mmol) as a brown solid.

TLC R_{f} 0.60 (CH₂Cl₂-MeOH-0.880NH₃, 90:10:1). ¹H (CD₃OD, 400 MHz) δ 1.4 (9H, s), 7.1 (1H, dd), 7.5 (1H, dd), 7.7 (1H, d), 7.8 (1H, d), 8.0(1H, d), 8.1 (1H, s), 9.1 (1H, s) ppm.

LRMS 475 (MH⁺).

The silica gel column was then eluted with MeOH and the combined washings were concentrated in vacuo to give an off-white solid. This was dissolved in a solution of EtOH saturated with HCl gas and the mixture stirred at room temperature. The solvents were evaporated in vacuo and the residue was then dissolved in EtOAc-MeOH, filtered and again evaporated in vacuo. The solid was triturated with water and then dried to give 2-{[(4-chloro-1-guanidino-7-isoquinolinyl)sulphonyl]amino}benzoic acid hydrochloride (11.8 mg, 0.02 mmol) as a pale yellow solid.

mp>280° C. (dec).

¹H (CD₃OD, 400 MHz) δ 7.0 (1H, dd), 7.3 (1H, dd), 7.65 (1H, d), 7.8 (1H, d), 8.1 (1H, d), 8.2 (1H, d), 8.3 (1H, s), 8.9 (1H, s)ppm.

LRMS 420,422 (M⁺), 421 (MH⁺).

Anal. Found: C, 43.58; H, 3.37; N, 14.65. Calc for C₁₇H₁₄ClN₅O₄S.1.0HCl.0.7H₂O: C, 43.54, H, 3.53; N, 14.94.

### Example 2

### (a) tert-Butyl 3-{[(4-chloro-1-guanidino-7-isoquinolinyl)sulphonyl]amino}benzoate

### (b) 3-{[(4-Chloro-1-guanidino-7-isoquinolinyl)sulphonyl]amino}benzoic acid trifluoroacetate

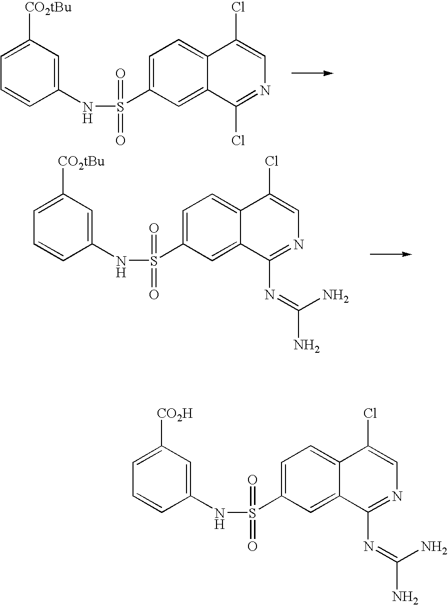

Guanidine hydrochloride (140 mg, 1.47 mmol) was added in one portion to a suspension of NaH (44 mg, 80% dispersion by wt in mineral oil, 1.47 mmol) in DMSO (4.0 mL) and the mixture was heated at 60° C. under N₂ for 30 min. A solution of tert-butyl 3-{[(1,4-dichloro-7-isoquinolinyl)-sulphonyl]amino}benzoate (280 mg, 0.59 mmol) in DMSO (2.0 mL) was added and the mixture heated at 90° C. for 18 h. The cooled mixture was poured into water (50 mL), extracted with EtOAc (×3) and the combined organic phase was then evaporated in vacuo. The residue was purified by column chromatography upon silica gel using CH₂Cl₂-MeOH-0.880NH₃ (97:3:0.3 to 95:5:0.5) as eluant to give tert-butyl 3-{[(4-chloro-1-guanidino-7-isoquinolinyl)sulphonyl]amino}benzoate (64 mg, 0.13 mmol) as a tan solid.

mp>142° C. (dec).

¹H (CD₃OD, 400 MHz) δ 1.5 (9H, s), 7.25-7.35 (2H, m), 7.65-7.7 (2H, m), 7.95 (1H, d), 8.05 (1H, d), 8.1 (1H, s), 9.1 (1H, s) ppm.

LRMS 475 (MH⁺).

Anal. Found: C, 51.07; H, 4.55; N, 13.94. Calc for C₂₁H₂₂ClN₅O₄S.0.23CH₂Cl₂: C, 51.46; H, 4.57; N, 14.13.

tert-Butyl 3-{[(4-chloro-1-guanidino-7-isoquinolinyl)sulphonyl]amino}benzoate (30 mg, 0.063 mmol) was dissolved in CF₃CO₂H (1.0 mL) and the mixture stirred at room temperature for 1 h. The mixture was diluted with PhMe and the solvents were evaporated in vacuo. The residue was triturated with Et₂O and then azeotroped with CH₂Cl₂ to give 3-{[(4-chloro-1-guanidino-7-isoquinolinyl)sulphonyl]amino}-benzoic acid trifluoroacetate (29 mg, 0.055 mmol) as an off-white solid.

mp>180° C. (dec).

¹H (CD₃OD, 400 MHz) δ7.2-7.35 (2H, m), 7.55 (1H, d), 7.65 (1H, s), 8.15 (1H, d), 8.3 (1H, d), 8.35 (1H, s), 8.85 (1H, s) ppm.

LRMS 419, 421 (MH⁺).

Anal. Found: C, 42.51; H, 3.07; N, 13.19. Calc for C₁₇H₁₄ClN₅O₄S.1.0CF₃CO₂H: 42.75; H, 2.83; N, 13.12.

### Example 3

### (a) Methyl 3-{[(4-chloro-1-guanidino-7-isoquinolinyl)sulphonyl]amino}-4-methoxybenzoate

### (b) 3-{[(4-Chloro-1-guanidino-7-isoquinolinyl)sulphonyl]amino}-4-methoxybenzoic acid hydrochloride

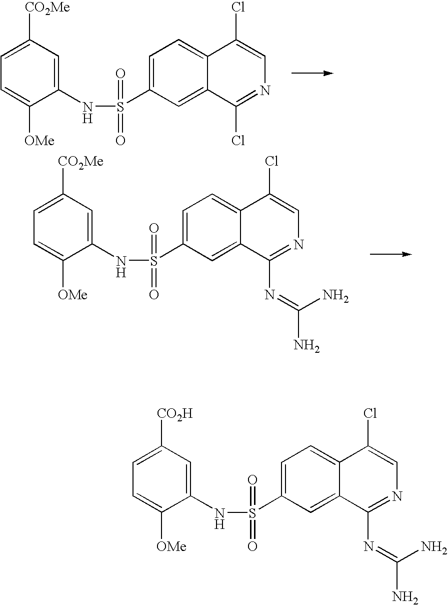

Guanidine hydrochloride (179.8 mg, 1.88 mmol) was added in one portion to a suspension of NaH (54.9 mg, 80% dispersion by wt in mineral oil, 1.83 mmol) in DMSO (10 mL) and the mixture was heated at 60° C. under N₂ for 20 min. Methyl 3-{[(1,4-dichloro-7-isoquinolinyl)sulphonyl]amino}-4-methoxybenzoate (238.6 mg, 0.541 mmol) was added and the mixture heated at 90° C. for 24 h. The solvents were evaporated in vacuo and the residue was purified by column chromatography upon silica gel using CH₂Cl₂-MeOH-0.880NH₃ (97:3:0.3 to 90:10:1) as eluant to give methyl 3-{[(4-chloro-1-guanidino-7-isoquinolinyl)sulphonyl]amino}-4-methoxybenzoate (203.2 mg, 0.43 mmol) as a pale yellow solid.

mp 134-137° C. (dec).

¹H (DMSO-d₆, 300 MHz) δ 3.45 (3H, s), 3.8 (3H, s), 6.95 (1H, d), 7.05-7.4 (4H, br s), 7.7 (1H, d), 7.8 (1H, s), 8.0 (2H, s), 8.1 (1H, s), 9.05 (1H, s), 9.9 (1H, br s) ppm.

LRMS 464, 466 (MH⁺).

Anal. Found: C, 48.37; H, 3.81; N, 14.75. Calc for C₁₉H₁₈ClN₅O₅S.0.15CH₂Cl₂: C, 48.26; H, 3.87; N, 14.69.

An aqueous solution of NaOH (0.7 mL, 1.0 M, 0.7 mmol) was added slowly to a stirred solution of methyl 3-{[(4-chloro-1-guanidino-7-isoquinolinyl)sulphonyl]amino}-4-methoxybenzoate (52.2 mg, 0.113 mmol) in dioxane (2.5 mL) and the mixture stirred at room temperature for 1.5 h, and then at 70° C. for 3 h. The mixture was cooled to room temperature, dilute HCl (2 mL, 2 N) was added, the solvents were evaporated in vacuo and the residue was dried by azeotroping with i-PrOH (×3). The solid was extracted with hot i-PrOH (×4), the combined organic extracts were filtered, and the solvents were evaporated in vacuo. The residue was triturated with Et₂O to give 3-{[(4-chloro-1-guanidino-7-isoquinolinyl)sulphonyl]amino}-4-methoxybenzoic acid hydrochloride (29 mg, 0.055 mmol) as a solid.

mp 258° C. (dec).

¹H (DMSO-d₆, 300 MHz) δ 3.45 (3H, s), 6.95 (1H, d), 7.7 (1H, d), 7.8 (1H, s), 8.3-8.7 (4H, br s), 8.3 (1H, d), 8.4 (1H, d), 8.45 (1H, s), 8.9 (1H, s), 10.05 (1H, br s), 10.9 (1H, br s), 12.75 (1H, br s) ppm.

LRMS 450 (MH⁺).

Anal. Found: C, 44.50; H, 4.60; N, 12.17. Calc for C₁₈H₁₆ClN₅O₅S.1.0HCl.1.0(CH₃)₂CHOH.1.0H₂O: C, 44.69; H, 4.82; N, 12.41.

### Example 4

### (a) N-[(4-chloro-1-guanidino-7-isoquinolinyl)sulphonyl]glycine t-butyl ester hydrochloride

### (b) N-[(4-Chloro-1-guanidino-7-isoquinolinyl)sulphonyl]glycine trifluoroacetate

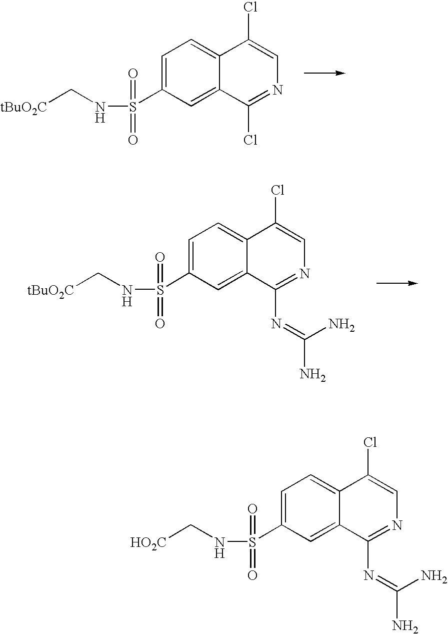

NaH (29 mg, 80% dispersion by wt in mineral oil, 0.97 mmol) was added in one portion to a stirred solution of guanidine hydrochloride (146 mg, 1.52 mmol) in DMSO (2.0 mL) and the mixture was heated at 60° C. under N₂ for 30 min. N-[(1,4-Dichloro-7-isoquinolinyl)sulphonyl]glycine t-butyl ester (150 mg, 0.38 mmol) was added and the mixture heated at 90° C. for 9 h. The cooled mixture was diluted with water (30 mL), extracted with EtOAc (4×20 mL) and the combined organic extracts were washed with water, brine, dried (Na₂SO₄) and evaporated in vacuo. The residue was dissolved in Et₂O and a solution of HCl in Et₂O (1 M) was added to give a sticky precipitate. The Et₂O was decanted and the residue triturated with EtOAc to give a white solid. Filtration with EtOAc and Et₂O washing gave N-[(4-chloro-1-guanidino-7-isoquinolinyl)sulphonyl]glycine t-butyl ester hydrochloride (68 mg, 0.14 mmol).

mp 172-175° C.

¹H (DMSO-d₆, 300 MHz) δ 1.2 (9H, s), 3.75 (2H, s), 8.3 (1H, d), 8.35-8.4 (2H, m), 8.5 (1H, s), 8.5-8.9 (4H, br), 9.1 (1 H, s), 11.3 (1H, br s) ppm.

LRMS 414, 416 (MH⁺).

Anal. Found: C, 42.45; H, 4.92; N, 14.76. Calc for C₁₆H₂₀ClN₅O₄S.1.0HCl.0.33H₂O.0.2EtOAc: C, 42.58; H, 4.95; N, 14.78.

N-[(4-Chloro-1-guanidino-7-isoquinolinyl)sulphonyl]glycine t-butyl ester hydrochloride (50 mg, 0.11 mmol) was dissolved in CF₃CO₂H (1.0 mL) and the mixture stirred at room temperature for 1.5 h. The mixture was diluted with PhMe and the solvents were evaporated in vacuo. The residue was triturated with Et₂O and EtOAc to give N-[(4-chloro-1-guanidino-7-isoquinolinyl)sulphonyl]glycine trifluoroacetate (36 mg, 0.073 mmol) as a white powder.

¹H (CF₃CO₂D, 400 MHz) δ 4.1 (2H, s), 8.25 (1H, d), 8.3 (1H, s), 8.55 (1H, d), 9.0 (1H, s) ppm.

LRMS 358 (MH⁺), 715 (M₂H⁺).

Anal. Found: C, 36.25; H, 2.86; N, 14.28. Calc for C₁₂H₁₂ClN₅O₄S.1.0CF₃CO₂H.0.2EtOAc: C, 36.32; H, 3.01; N, 14.31.

### Example 5

### (a) N-[(4-chloro-1-guanidino-7-isoquinolinyl)sulphonyl]-β-alanine t-butyl ester

### (b) N-[(4-Chloro-1-guanidino-7-isoquinolinyl)sulphonyl]-β-alanine trifluoroacetate

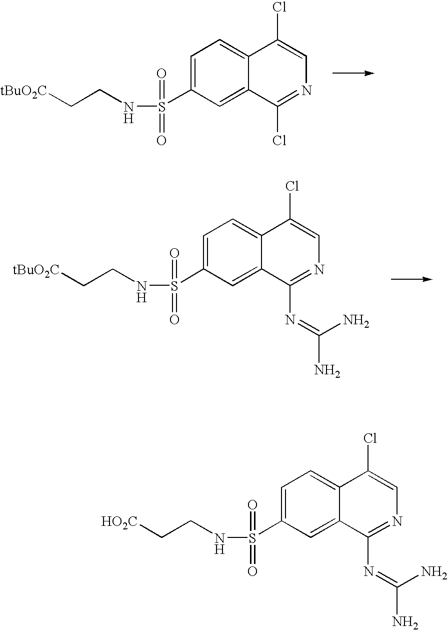

Guanidine hydrochloride (140 mg, 1.46 mmol was added in one portion to a stirred suspension of NaH (35 mg, 80% dispersion by wt in mineral oil, 1.17 mmol) in DME (8.0 mL) and the mixture was heated at 30° C. under N₂ for 30 min. N-[(1,4-Dichloro-7-isoquinolinyl)sulphonyl]-β-alanine t-butyl ester (150 mg, 0.37 mmol) was added and the mixture heated at 90° C. for 18 h. The cooled mixture was diluted with EtOAc, washed with water, brine, dried (MgSO₄) and evaporated in vacuo. The residue was purified by column chromatography upon silica gel using CH₂Cl₂-MeOH-0.880NH₃ (97:3:0.3 to 95:5:0.5) as eluant to give N-[(4-chloro-1-guanidino-7-isoquinolinyl)sulphonyl]-β-alanine t-butyl ester (75 mg, 0.175 mmol) as a yellow foam

mp>180° C. (dec).

¹H (DMSO-d₆, 300 MHz) δ 1.35 (9H, s), 2.3 (2H, t), 2.9 (2H, dt), 7.1-7.4 (4H, br), 7.8 (1H, br t), 8.05 (2H, s), 8.1 (1H, s), 9.1 (1H, s) ppm.

LRMS 428 (MH⁺).

Anal. Found: C, 47.32; H, 5.24; N, 16.02. Calc for C₁₇H₂₂ClN₅O₄S.0.2H₂O: C, 47.32; H, 5.23; N, 16.23.

N-[(4-Chloro-1-guanidino-7-isoquinolinyl)sulphonyl]-β-alanine t-butyl ester (30 mg, 0.07 mmol) was dissolved in CF₃CO₂H (1.0 mL) and the mixture stirred at room temperature for 1 h. The mixture was evaporated in vacuo, azeotroping with PhMe, MeOH and then CH₂Cl₂, to give N-[(4-chloro-1-guanidino-7-isoquinolinyl)sulphonyl]-β-alanine trifluoroacetate (32 mg, 0.066 mmol) as a white solid.

mp>200° C. (dec).

¹H (DMSO-d₆+D₂0, 400 MHz) δ 2.35 (2H, t), 3.0 (2H, t), 8.2 (1H, d),8.3 (1H, d), 8.4 (1H, s), 9.1 (1H, s) ppm.

LRMS 372 (MH⁺).

Anal. Found: C, 37.38; H, 3.11; N, 14.52. Calc for C₁₃H₁₄ClN₅O₄S.1.0CF₃CO₂H: C, 37.08; H, 3.11; N, 14.42.

### Example 6

### (a) N-[(4-chloro-1-guanidino-7-isoquinolinyl)sulphonyl]-N-methylglycine t-butyl ester

### (b) N-[(4-Chloro-1-guanidino-7-isoquinolinyl)sulphonyl]-N-methylglycine bis(trifluoroacetate)

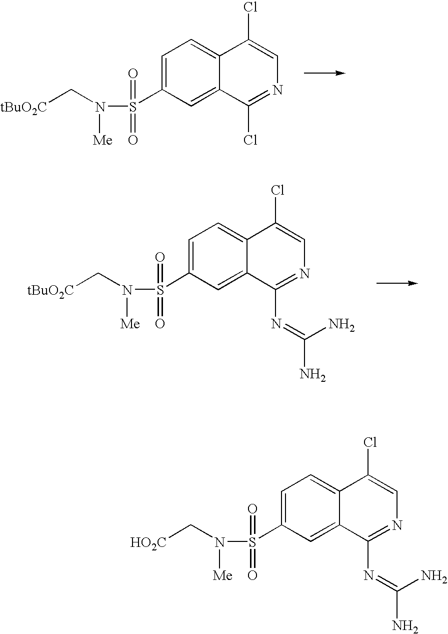

Guanidine hydrochloride (286 mg, 2.99 mmol was added in one portion to a stirred suspension of NaH (77.5 mg, 80% dispersion by wt in mineral oil, 2.58 mmol) in DME (2.0 mL) and the mixture was heated at 50° C. under N₂ for 20 min. A solution of N-[(1,4-dichloro-7-isoquinolinyl)sulphonyl]-N-methylglycine t-butyl ester (393 mg, 0.97 mmol) in DME (10 mL) was added and the mixture heated at 90° C. for 2 h. The solvents were evaporated in vacuo and the residue was purified by column chromatography upon silica gel using CH₂Cl₂-MeOH-0.880NH₃ (97:3:0.3) as eluant to give N-[(4-chloro-1-guanidino-7-isoquinolinyl)sulphonyl]-N-methylglycine t-butyl ester (260 mg, 0.607 mmol) as an off-white foam

mp 84° C.

¹H (DMSO-d₆, 300 MHz) δ 1.3 (9H, s), 2.85 (3H, s), 3.95 (2H, s), 7.0-7.5 (4H, br), 8.0 (1H, d), 8.05 (1H, d), 8.1 (1H, s), 9.05 (1H, s) ppm.

LRMS 427 (MH⁺), 855 (M₂H⁺).

Anal. Found: C, 47.92; H, 5.38; N, 15.07. Calc for C₁₇H₂₂ClN₅O₄S: C, 47.72; H, 5.18; N, 16.37.

N-[(4-Chloro-1-guanidino-7-isoquinolinyl)sulphonyl]-N-methylglycine t-butyl ester (255 mg, 5.96 mmol) was dissolved in CF₃CO₂H (4.0 mL) and CH₂Cl₂ (2.0 mL), and the mixture stirred at room temperature for 1 h. The mixture was diluted with PhMe and the solvents were evaporated in vacuo to give N-[(4-chloro-1-guanidino-7-isoquinolinyl)sulphonyl]-N-methylglycine bis(trifluoroacetate) (349 mg, 0.56 mmol) as a white powder.

mp 240-242° C. (dec).

¹H (DMSO-d₆, 300 MHz) δ 2.9 (3H, s), 4.05 (2H, s), 8.3 (1H, d), 8.4 (1H, d), 8.4-8.7 (4H, br), 8.5 (1H, s), 8.9 (1H, s) ppm.

LRMS 372, 374 (MH⁺), 744 (M₂H⁺).

Anal. Found: C, 36.26; H, 3.10; N, 11.04. Calc for Cl₃H₁₄ClN₅O₄S.2.0CF₃CO₂H.0.3PhMe: C, 36.56; H, 2.96; N, 11.16.

### Example 7

### (a) N-[(4-chloro-1-guanidino-7-isoquinolinyl)sulphonyl]-N-phenylglycine t-butyl ester

### (b) N-[(4-Chloro-1-guanidino-7-isoquinolinyl)sulphonyl]-N-phenylglycine trifluoroacetate

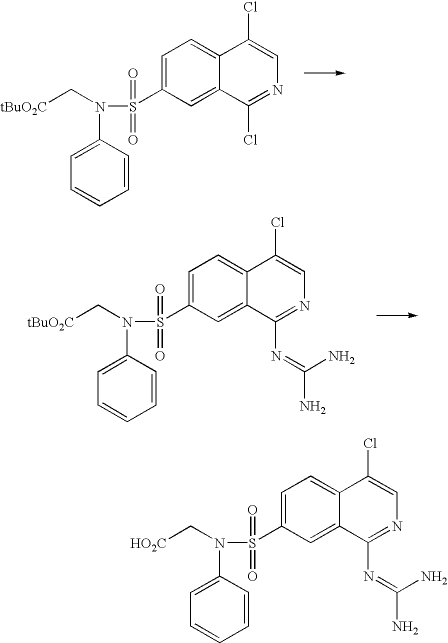

NaH (32 mg, 80% dispersion by wt in mineral oil, 1.07 mmol) was added in one portion to a stirred suspension of guanidine hydrochloride (164 mg, 1.71 mmol) in DME (5.0 mL) and the mixture was heated at 60° C. under N₂ for 30 min. N-[(1,4-Dichloro-7-isoquinolinyl)sulphonyl]-N-phenylglycine t-butyl ester (200 mg, 0.43 mmol) was added and the mixture heated at 95° C. for 6 h. The solvents were evaporated in vacuo and the residue was purified by column chromatography upon silica gel using CH₂Cl₂-MeOH-0.880NH₃ (97:3:0.3 to 95:5:0.5) as eluant to give N-[(4-chloro-1-guanidino-7-isoquinolinyl)sulphonyl]-N-phenylglycine t-butyl ester (28 mg, 0.057 mmol) as a yellow resin.

¹H (DMSO-d₆, 300 MHz) δ 1.3 (9H, s), 4.45 (2H, s), 7.2-7.3 (2H, m), 7.2-7.4 (4H, br), 7.3-7.4 (3H, m), 7.9 (1H, d), 8.0 (1H, d), 8.1 (1H, s), 8.95 (1H, s) ppm.

LRMS 490,492 (MH⁺), 981 (M₂H⁺).

N-[(4-Chloro-1-guanidino-7-isoquinolinyl)sulphonyl]-N-phenylglycine t-butyl ester (25 mg, 0.05 mmol) was dissolved in CF₃CO₂H (1.0 mL) and the mixture stirred at room temperature for 2 h. The mixture was concentrated in vacuo, azeotroping with PhMe, and the residue triturated with Et₂O to give N-[(4-chloro-1-guanidino-7-isoquinolinyl)sulphonyl)-N-phenylglycine trifluoroacetate (13 mg, 0.23 mmol) as a pale yellow powder.

mp 218-223° C.

¹H (DMSO-d₆, 300 MHz) δ 4.5 (2H, s), 7.1-7.2 (2H, d), 7.25-7.4 (3H, m), 7.8-8.4 (4H, br), 8.0 (1H, d), 8.2 (1H, d), 8.35 (1H, s), 8.9 (1H, s) ppm.

LRMS 434, 436 (MH⁺), 744 (M₂H⁺).

Anal. Found: C, 42.55; H, 3.39; N, 11.90. Calc for C₁₈H₁₆ClN₅O₄S.1.0CF₃CO₂H.H₂O.0.2Et₂O: C, 42.74; H, 3.52; N, 12.22.

### Example 8

### (a) N-[(4-chloro-1-guanidino-7-isoquinolinyl)sulphonyl]-N-(cyclopentylmethyl)-glycine t-butyl ester

### (b) N-[(4-Chloro-1-guanidino-7-isoquinolinyl)sulphonyl]-N-(cyclopentylmethyl)glycine

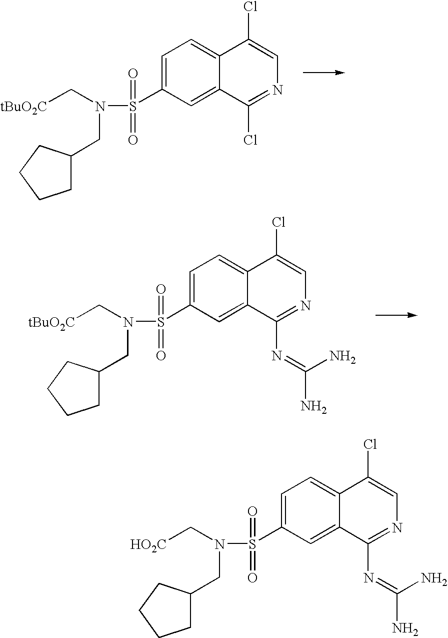

Guanidine hydrochloride (96 mg, 1.00 mmol was added in one portion to a stirred suspension of NaH (19 mg, 80% dispersion by wvt in mineral oil, 0.63 mmol) in DME (5.0 mL) and the mixture was heated at 60° C. under N₂ for 30 min. A solution of N-[(1,4-dichloro-7-isoquinolinyl)sulphonyl]-N-(cyclopentylmethyl)glycine t-butyl ester (120 mg, 0.25 mmol) in DME (5.0 mL) was added and the mixture heated at 90° C. for 3 h. The solvents were evaporated in vacuo, the residue was dissolved with EtOAc (200 mL), and washed with aqueous NH₄Cl (150 mL), dried (MgSO₄) and evaporated in vacuo. The residue was purified by column chromatography upon silica gel using pentane-EtOAc (100:0 to 40:60) as eluant to give N-[(4-chloro-1-guanidino-7-isoquinolinyl)sulphonyl]-N-(cyclopentylmethyl)-glycine t-butyl ester (60 mg, 0.12 mmol).

¹H (CDCl₃, 400 MHz) δ 1.1-1.25 (2H, m), 1.35 (9H, s), 1.45-1.7 (4H, m), 1.7-1.8 (2H, m), 2.1 (1H, m), 3.25 (2H, d), 4.0 (2H, s), 8.05 (1H, d), 8.1 (1H, d), 8.15 (1H, s), 9.2 (1H, s) ppm.

LRMS 496 (MH⁺).

Anal. Found: C, 52.99; H, 6.07; N, 13.82. Calc for C₂₂H₃₀ClN₅O₄S: C, 53.38; H, 5.90; N, 14.15.

A solution of HCl (2 mL, 2 M, 4 mmol) was added to a solution of N-[(4-chloro-1-guanidino-7-isoquinolinyl)sulphonyl]-N-(cyclopentylmethyl)glycine t-butyl ester (50 mg, 0.10 mmol) in dioxane (4.0 mL) and the mixture was heated at 60° C. for 24 h. The solvents were evaporated in vacuo, and the residue triturated with dichloromethane to give N-[(4-chloro-1-guanidino-7-isoquinolinyl)sulphonyl]-N-(cyclopentylmethyl)glycine hydrochloride (40 mg, 0.080 mmol) as a white solid.

mp 139-142° C.

¹H (CD₃OD, 400 MHz) δ 1.2-1.3 (2H, m), 1.5-1.7 (4H, m), 1.7-1.8 (2H, m), 2.2 (1H, m), 3.65 (2H, d), 4.2 (2H, s), 8.35 (1H, d), 8.45 (1H, s), 8.45 (1H, d), 8.9 (1H, s) ppm.

LRMS 440 (MH⁺).

Anal. Found: C, 43.48; H, 5.32; N, 12.91. Calc for C₁₈H₂₂ClN₅O₄S.1.0HCl.1.0H₂O.0.05CH₂Cl₂.0.05 dioxane: C, 43.17; H, 5.11; N, 13.92.

### Example 9

### (a) N-[(4-chloro-1-guanidino-7-isoquinolinyl)sulphonyl]-N-cyclohexylmethyl)glycine t-butyl ester

### (b) N-[(4-Chloro-1-guanidino-7-isoquinolinyl)sulphonyl]-N-(cyclohexylmethyl)glycine hydrochloride

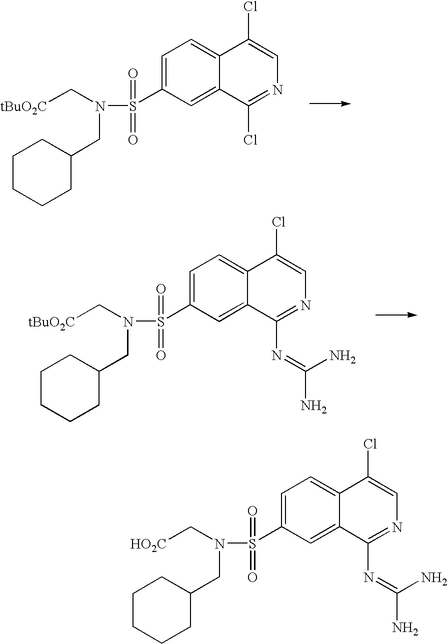

Guanidine hydrochloride (125 mg, 1.31 mmol was added in one portion to a stirred suspension of NaH (25 mg, 80% dispersion by wt in mineral oil, 0.82 mmol) in DME (10 mL) and the mixture was heated at 60° C. under N₂ for 30 min. N-[(1,4-Dichloro-7-isoquinolinyl)sulphonyl]-N-(cyclohexylmethyl)-glycine t-butyl ester (160 mg, 0.33 mmol) was added and the mixture heated at 80-90° C. for 2.5 h. The solvents were evaporated in vacuo, the residue was dissolved with EtOAc (200 mL), and washed with aqueous NH₄Cl (150 mL), dried (MgSO₄) and evaporated in vacuo. The residue was purified by column chromatography upon silica gel using pentane-EtOAc (100:0 to 40:60) as eluant to give N-[(4-chloro-1-guanidino-7-isoquinolinyl)sulphonyl]-N-(cyclohexylmethyl)glycine t-butyl ester (65 mg, 0.127 mmol) as an off-white foam.

¹H (CDCl₃, 400 MHz) δ 0.8-0.95 (2H, m), 1.1-1.25 (3H, m), 1.3 (9H, s), 1.6-1.8 (6H, m), 3.1 (2H, d), 4.0 (2H, s), 8.0 (1H, d), 8.1 (1H, d), 8.15 (1H, s), 9.2 (1H, s) ppm.

LRMS 510 (MH⁺).

Anal. Found: C, 54.21; H, 6.46; N, 13.46. Calc for C₂₃H₃₂ClN₅O₄S: C, 54.16; H, 6.32; N, 13.73.

A solution of HCl (2 mL, 2 M, 4 mmol) was added to a solution of N-[(4-chloro-1-guanidino-7-isoquinolinyl)sulphonyl]-N-(cyclohexylmethyl)glycine t-butyl ester (53 mg, 0.10 mmol) in dioxane (4.0 mL). The mixture was stirred at 23° C. for 18 h and then heated at 50-60° C. for 16 h. On cooling, a white precipitate crashed out of solution. The solid was collected by filtration, triturated with EtOAc and then dried under vacuum to give N-[(4-chloro-1-guanidino-7-isoquinolinyl)sulphonyl]-N-(cyclohexylmethyl)glycine hydrochloride (26 mg, 0.057 mmol).

¹H (CDCl₃, 400 MHz) δ 0.8-1.0 (2H, m), 1.1-1.3 (3H, m), 1.55-1.8 (6H, m), 3.2 (2H, d), 4.15 (2H, s), 8.3 (1H, d), 8.45 (1H, d), 8.45 (1H, s), 8.9 (1H, s) ppm.

LRMS 454, 456 (MH⁺).

Anal. Found: C, 44.70; H, 5.15; N, 13.56. Calc for C₂₃H₃₂ClN₅O₄S.HCl.H₂O: C, 44.89; H, 5.36; N, 13.77.

### Example 10

### (a) N-[(4-chloro-1-guanidino-7-isoquinolinyl)sulphonyl]-N-benzylglycine t-butyl ester

### (b) N-[(4-Chloro-1-guanidino-7-isoquinolinyl)sulphonyl]-N-benzylglycine trifluoroacetate

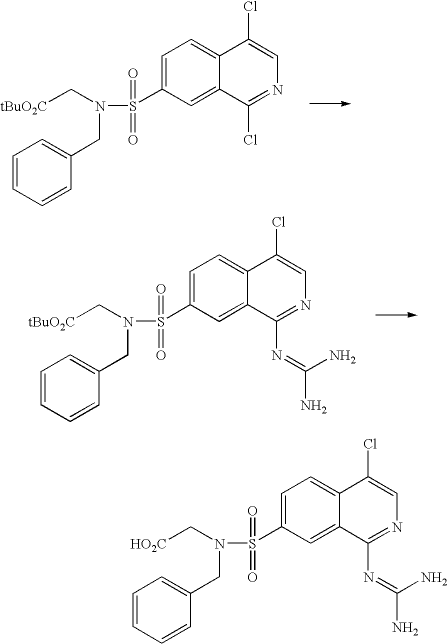

Guanidine hydrochloride (180 mg, 1.88 mmol) was added in one portion to a suspension of NaH (45 mg, 80% dispersion by wt in mineral oil, 1.5 mmol) in DME (11 mL) and the mixture was heated at 60° C. under N₂ for 30 min. N-[(1,4-Dichloro-7-isoquinolinyl)sulphonyl]-N-benzylglycine t-butyl ester (225 mg, 0.467 mmol) was added and the mixture heated at 90° C. for 18 h. The cooled mixture was poured into water, extracted with EtOAc (×3) and the combined organic phase was then washed with water, dried (Na₂SO₄) and evaporated in vacuo. The residue was purified by column chromatography upon silica gel using CH₂Cl₂-MeOH-0.880NH₃ (97:3:0.3) as eluant to give N-[(4-chloro-1-guanidino-7-isoquinolinyl)sulphonyl]-N-benzylglycine t-butyl ester (172 mg, 0.34 mmol) as a yellow foam.

mp>150° C. (dec).

¹H (DMSO-d₆, 400 MHz) δ 1.2 (9H, s), 3.8 (2H, s), 4.45 (2H, s), 7.1-7.4 (4H, br), 7.2-7.35 (5H, m), 8.0 (1H, d), 8.1 (1H, d), 8.1 (s, 1H), 9.1 (1H, s) ppm.

LRMS 504, 506 (MH⁺).

Anal. Found: C, 55.19; H, 5.55; N, 13.23. Calc for C₂₃H₂₆ClN₅O₄S.0.1C₆H₁₄: C, 55.30; H, 5.39; N, 13.66.

N-[(4-Chloro-1-guanidino-7-isoquinolinyl)sulphonyl]-N-benzylglycine t-butyl ester (50 mg, 0.10 mmol) was dissolved in CF₃CO₂H (1.0 mL) and the mixture stirred at room temperature for 1 h. The mixture was diluted with PhMe and the solvents were evaporated in vacuo. The residue was azeotroped with PhMe and then CH₂Cl₂ to give N-[(4-chloro-1-guanidino-7-isoquinolinyl)sulphonyl]-N-benzylglycine trifluoroacetate (52 mg, 0.10 mmol) as a white solid.

mp 274° C. (dec).

¹H (DMSO-d₆, 400 MHz) δ 3.95 (2H, s), 4.5 (2H, s), 7.2-7.35 (5H, m), 8.3 (1H, d), 8.35 (1H, d), 8.4-8.6 (4H, br), 8.45 (1H, s), 8.9 (1H, s), 10.6 (1H, br), 12.7 (1H, br) ppm.

LRMS 448, 450 (MH⁺), 497 (M₂H⁺).

Anal. Found: C, 43.96; H, 3.39; N, 11.87. Calc for C₁₉H₁₈ClN₅O₄S.1.0CF₃CO₂H.0.5H₂O: C, 44.18; H, 3.53; N, 12.27.

### Example 11

### (a) N-[(4-chloro-1-guanidino-7-isoquinolinyl)sulphonyl]-N-(2-methylbenzyl)glycine t-butyl ester

### (b) N-[(4-Chloro-1-guanidino-7-isoquinolinyl)sulphonyl]-N-(2-methylbenzyl)glycine trifluoroacetate

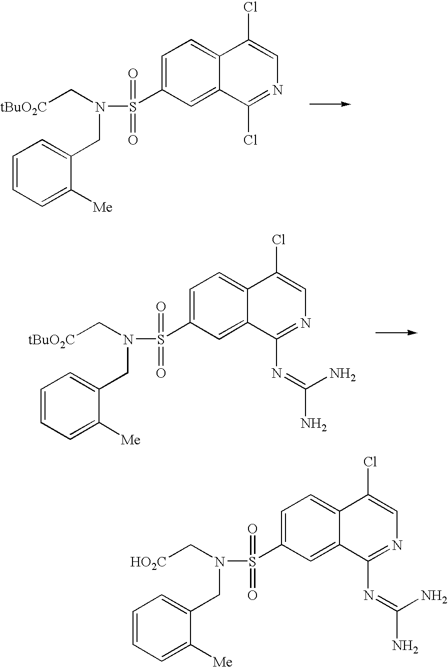

Guanidine hydrochloride (120 mg, 1.26 mmol) was added in one portion to a suspension of NaH (32 mg, 80% dispersion by wt in mineral oil, 1.06 mmol) in DME (10 mL) and the mixture was heated at 60° C. under N₂ for 30 min. N-[(1,4-Dichloro-7-isoquinolinyl)sulphonyl]-N-(2-methylbenzyl)glycine t-butyl ester (200 mg, 0.405 mmol) was added and the mixture heated at 90° C. for 2 h. The cooled mixture was diluted with EtOAc, washed with water, brine, dried (Na₂SO₄) and evaporated in vacuo. The residue was purified by column chromatography upon silica gel using pentane-CH₂Cl₂ (50:50), then CH₂Cl₂, and finally CH₂Cl₂-MeOH-0.880NH₃ (95:5:0.5) as eluant to give N-[(4-chloro-guanidino-7-isoquinolinyl)sulphonyl]-N-(2-methylbenzyl)glycine t-butyl ester (94 mg, 0.18 mmol) as an off-white solid.

mp>110° C. (dec).

¹H (CDCl₃, 400 MHz) δ 1.25 (9H, s), 2.3 (3H, s), 3.8 (2H, s), 4.6 (2H, s), 7.1-7.2 (4H, m), 8.05 (1H, d), 8.1 (1H, d), 8.15 (s, 1H), 9.3 (1H, s) ppm.

LRMS 518, 520 (MH⁺).

Anal. Found: C, 56.21; H, 5.83; N, 12.57. Calc for C₂₄H₂₈ClN₅O₄S.0.3H₂O.0.25C₆H₁₅: C, 56.20; H, 5.94; N, 12.85.

N-[(4-Chloro-1-guanidino-7-isoquinolinyl)sulphonyl]-N-(2-methylbenzyl)glycine t-butyl ester (30 mg, 0.058 mmol) was dissolved in CF₃CO₂H (1.0 mL) and the mixture stirred at room temperature for 1 h.

The mixture was diluted with PhMe and the solvents were evaporated in vacuo. The residue was azeotroped with PhMe and then Et₂O to give N-[(4-chloro-1-guanidino-7-isoquinolinyl)sulphonyl]-N-(2-methylbenzyl)glycine trifluoroacetate (29 mg, 0.05 mmol) as an off-white solid.

mp>150° C. (dec).

¹H (CD₃OD, 400 MHz) δ 2.3 (3H, s), 3.95 (2H, s), 4.7 (2H, s), 7.05-7.2 (4H, m), 8.35 (1H, d), 8.45 (1H, s), 8.45 (1H, d), 8.9 (1H, s) ppm.

LRMS 462, 464 (MH⁺).

Anal. Found: C, 45.51; H, 3.95; N, 11.36. Calc for C₂₀H₂₀ClN₅O₄S.1.0CF₃CO₂H.1.0H₂O.0.1PhMe: C, 45.20; H, 3.98; N, 11.61.

### Example 12

### (a) N-[(4-chloro-1-guanidino-7-isoquinolinyl)sulphonyl]-N-(2-methoxybenzyl)glycine t-butyl ester trifluoroacetate

### (b) N-[(4-Chloro-1-guanidino-7-isoquinolinyl)sulphonyl]-N-(2-methoxybenzyl)glycine trifluoroacetate

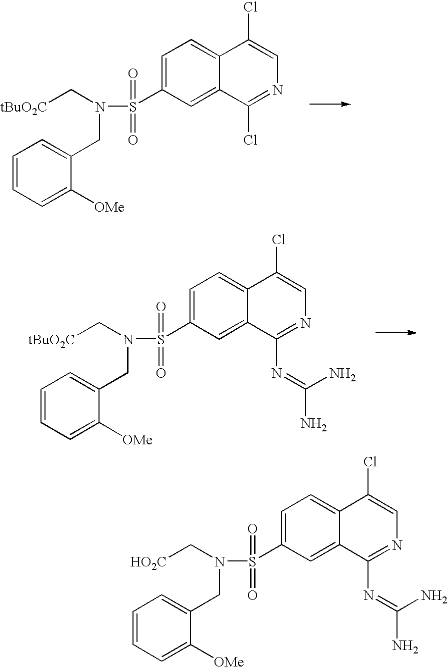

Guanidine hydrochloride (225 mg, 2.36 mmol) was added in one portion to a stirred suspension of NaH (44 mg, 80% dispersion by wt in mineral oil, 1.47 mmol) in DME (20 mL) and the mixture was heated at 60° C. under N₂ for 30 min. N-[(1,4-Dichloro-7-isoquinolinyl)sulphonyl]-N-(2-methoxybenzyl)glycine t-butyl ester (300 mg, 0.59 mmol) was added and the mixture heated at 90° C. for 2 h. The cooled mixture was poured into water and extracted with EtOAc (×3). The combined organic extracts were then washed with water, brine, dried (Na₂SO₄) and evaporated in vacuo. The residue was purified by column chromatography upon silica gel using hexane-EtOAc (80:20), and then CH₂Cl₂-MeOH-0.880NH₃ (95:5:0.5 to 90:10:1) as eluant to give the product as a yellow semi-solid. This semi-solid was dissolved in EtOAc, a solution of TFA (35 μL) in EtOAc (25 mL) was added and the solvents were evaporated in vacuo, azeotroping with PhMe. The residue was triturated with i-Pr₂O, the resulting white solid was collected by filtration, and then dried to give N-[(4-chloro-1-guanidino-7-isoquinolinyl)sulphonyl]-N-(2-methoxybenzyl)glycine t-butyl ester trifluoroacetate (219 mg, 0.338 mmol).

mp>197° C. (dec).

¹H (DMSO-d₆, 400 MHz) δ 1.25 (9H, s), 3.6 (3H, s), 4.0 (2H, s), 4.45 (2H, s), 6.8-6.9 (2H, m), 7.1-7.2 (2H, m), 8.3 (2H, s), 8.4-8.6 (4H, br s), 8.5 (s, 1H), 8.8 (1H, s) ppm.

LRMS 534, 536 (MH⁺).

Anal. Found: C, 48.33; H, 4.55; N, 10.52. Calc for C₂₄H₂₈ClN₅O₅S.1.0CF₃CO₂H: C, 48.18; H, 4.51; N, 10.81.

N-[(4-chloro-1-guanidino-7-isoquinolinyl)sulphonyl]-N-(2-methoxybenzyl)glycine t-butyl ester trifluoroacetate (150 mg, 0.231 mmol) was dissolved in CF₃CO₂H (1.0 mL) and the mixture stirred at room temperature for 40 min. The mixture was diluted with PhMe, concentrated in vacuo, azeotroping with PhMe, and the residue triturated with i-Pr₂O to give N-[(4-chloro-1-guanidino-7-isoquinolinyl)sulphonyl]-N-(2-methoxybenzyl)glycine trifluoroacetate (122 mg, 0.206 mmol) as a white solid.

mp>165° C. (dec).

¹H (DMSO-d₆, 400 MHz) δ 3.6 (3H, s), 4.0 (2H, s), 4.5 (2H, s), 6.8 (1H, d), 6.85 (1H, dd), 7.1-7.2 (2H, m), 8.3 (2H, s), 8.35-8.5 (4H, br s), 8.5 (s, 1H), 8.8 (1H, s) ppm.

LRMS 478, 480 (MH⁺).

Anal. Found: C, 44.64; H, 3.58; N, 11.83. Calc for C₂₀H₂₀ClN₅O₅S.1.0CF₃CO₂H: C, 44.69; H, 3.68; N, 11.63.

### Example 13

### (a) N-[(4-chloro-1-guanidino-7-isoquinolinyl)sulphonyl]-N3-methoxybenzyl)glycine t-butyl ester hydrochloride

### (b) N-[(4-Chloro-1-guanidino-7-isoquinolinyl)sulphonyl]-N-(3-methoxybenzyl)glycine

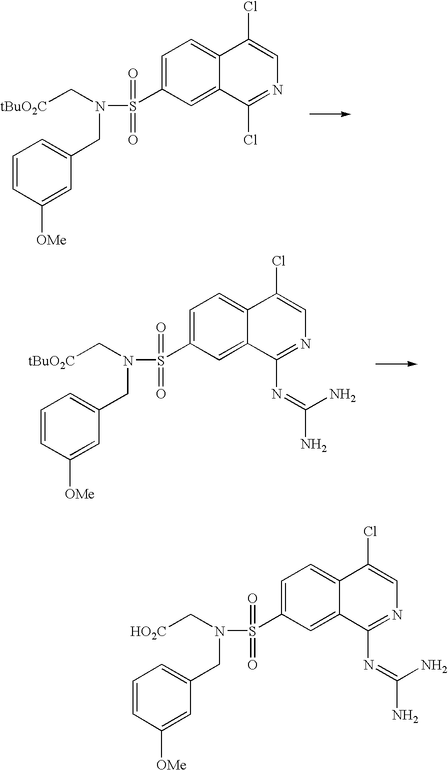

Guanidine hydrochloride (149 mg, 1.55 mmol) was added in one portion to a suspension of NaH (35 mg, 80% dispersion by wt in mineral oil, 1.16 mmol) in DME (10 mL) and the mixture was heated at 60° C. under N₂ for 30 min. N-[(1,4-Dichloro-7-isoquinolinyl)sulphonyl]-N-(3-methoxybenzyl)glycine t-butyl ester (200 mg, 0.39 mmol) was added and the mixture heated at 90° C. for 2 h. The cooled mixture was poured into water, extracted with EtOAc (×3), and the combined organic extracts were washed with brine, dried (Na₂SO₄) and evaporated in vacuo. The residue was dissolved in Et₂O-EtOAc and a solution of HCl in Et₂O (0.5 M) was added to give a precipitate. The solid was collected by filtration, triturated with EtOAc and then dried to give N-[(4-chloro-1-guanidino-7-isoquinolinyl)sulphonyl]-N-(3-methoxybenzyl)glycine t-butyl ester hydrochloride (124 mg, 0.21 mmol) as a white solid.

mp 203-205° C.

¹H (DMSO-d₆, 300 MHz) δ 1.2 (9H, s), 3.65 (3H, s), 4.05 (2H, s), 4.5 (2H, s), 6.7 (1H, s), 6.75-6.85 (2H, m), 7.2 (1H, dd), 8.3 (1H, d), 8.35 (1H, d), 8.5 (s, 1H), 9.3 (1H, s), 11.6 (1H, br s) ppm.

LRMS 534, 536 (MH⁺), 1069 (M₂H⁺).

Anal. Found: C, 50.22; H, 5.11; N, 12.23. Calc for C₂₄H₂₈ClN₅O₅S.1.0HCl: C, 56.52; H, 5.12; N, 12.28.

N-[(4-Chloro-1-guanidino-7-isoquinolinyl)sulphonyl]-N-(3-methoxybenzyl)glycine t-butyl ester hydrochloride (95 mg, 0.167 mmol) was dissolved in CF₃CO₂H (1.0 mL) and the mixture stirred at room temperature for 1 h. The mixture was diluted with PhMe and the solvents were evaporated in vacuo. The residue was dissolved in EtOAc and stirred at room temperature for 1 h. The resulting precipitate was collected by filtration, washed with Et₂O and dried to give N-[(4-chloro-1-guanidino-7-isoquinolinyl)sulphonyl]-N-(3-methoxybenzyl)glycine (65 mg, 0.128 mmol) as a white powder.

mp 290° C. (dec).

¹H (CF₃CO₂D, 400 MHz) δ 3.9 (3H, s), 4.3 (2H, s), 4.6 (2H, s), 6.9-7.0 (3H, m), 7.3 (1H, dd), 8.35 (1H, d), 8.45 (1H, s), 8.6 (1H, d), 8.95 (1H, s) ppm.

LRMS 477, 479 (MH⁺), 955 (M₂H⁺).

Anal. Found: C, 48.67; H, 4.09; N, 13.88. Calc for C₂₀H₂₀ClN₅O₅S.0.25CF₃CO₂H: C, 48.62; H, 4.03; N, 13.83.

### Example 14

### (a) N-[(4-chloro-1-guanidino-7-isoquinolinyl)sulphonyl]-N-(3-chlorobenzyl)glycine t-butyl ester hydrochloride

### (b) N-1(4-Chloro-1-guanidino-7-isoquinolinyl)sulphonyl]-N-(3-chlorobenzyl)glycine trifluoroacetate

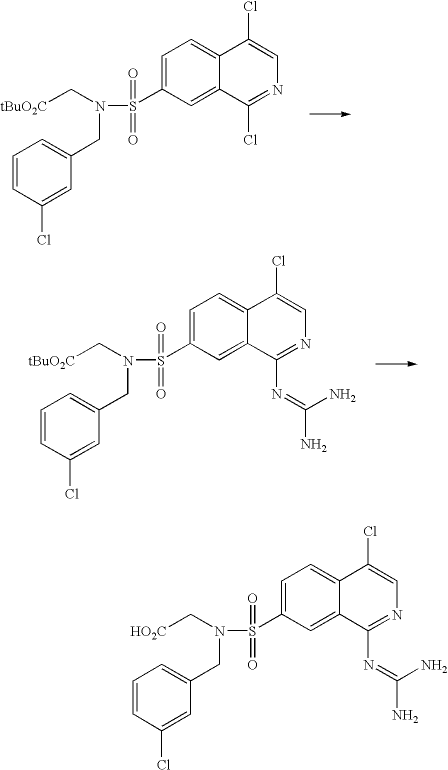

NaH (35 mg, 80% dispersion by wt in mineral oil, 1.16 mmol) was added in one portion to a suspension of guanidine hydrochloride (150 mg, 1.55 mmol) in DME (10 mL) and the mixture was heated at 60° C. under N2 for 30 min. N-[(1,4-Dichloro-7-isoquinolinyl)sulphonyl]-N-(3-chlorobenzyl)glycine t-butyl ester (185 mg, 0.36 mmol) was added and the mixture heated at 90° C. for 5 h. The cooled mixture was diluted with Et₂O, washed with water, dried (Na₂SO₄) and evaporated in vacuo. The residue was dissolved in Et₂O and a solution of HCl in Et₂O (1 M) was added to give a precipitate. The solvents were evaporated in vacuo, and the white solid triturated with EtOAc and then dried to give N-[(4-chloro-1-guanidino-7-isoquinolinyl)sulphonyl]-N-(3-chlorobenzyl)glycine t-butyl ester hydrochloride (85 mg, 0.145 mmol).

mp 203-205° C.

¹H (DMSO-d₆, 300 MHz) δ 1.2 (9H, s), 4.1 (2H, s), 4.55 (2H, s), 7.2-7.35 (4H, m), 8.3 (1H, d), 8.35 (1H, d), 8.5 (s, 1H), 9.3 (1H, s), 11.55 (1H, br s) ppm.

LRMS 538, 540 (MH⁺), 1076 (M₂H⁺).

Anal. Found: C, 47.04; H, 4.53; N, 11.82. Calc for C₂₃H₂₅Cl₂N₅O₄S.1.0HCl.0.5H₂O: C, 47.31; H, 4.66; N, 11.99.

N-[(4-Chloro-1-guanidino-7-isoquinolinyl)sulphonyl]-N-(3-chlorobenzyl)glycine t-butyl ester hydrochloride (60 mg, 0.104 mmol) was dissolved in CF₃CO₂H (0.5 mL) and the mixture stirred at room temperature for 1 h. The mixture was diluted with PhMe and the solvents were evaporated in vacuo. The residue was dissolved in Et₂O and stirred at room temperature for 1 h. The resulting precipitate was collected by filtration, washed with Et₂O and dried to give N-[(4-chloro-1-guanidino-7-isoquinolinyl)sulphonyl]-N-(3-chlorobenzyl)glycine trifluoroacetate (31 mg, 0.052 mmol) as a white solid.

mp 306-308° C.

¹H (CF₃CO₂D, 400 MHz) δ 4.3 (2H, s), 4.55 (2H, s), 7.0-7.1 (2H, m), 7.1-7.15 (2H, m), 8.25 (1H, d), 8.4 (1H, s), 8.5 (1H, d), 8.8 (1H, s) ppm.

LRMS 482, 484 (MH⁺), 496, 498 (MH⁺ of corresponding methyl ester).

Anal. Found: C, 42.60; H, 3.04; N, 12.03. Calc for C₁₉H₁₇Cl₂N₅O₄S.1.0CF₃CO₂H: C, 42.29, H, 3.04; N, 11.74.

### Example 15

### (a) N-[(4-chloro-1-guanidino-7-isoquinolinyl)sulphonyl]-N-(4-methoxybenzyl)glycine t-butyl ester hydrochloride

### (b) N-[(4-Chloro-1-guanidino-7-isoquinolinyl)sulphonyl]-N4-methoxybenzyl)glycine

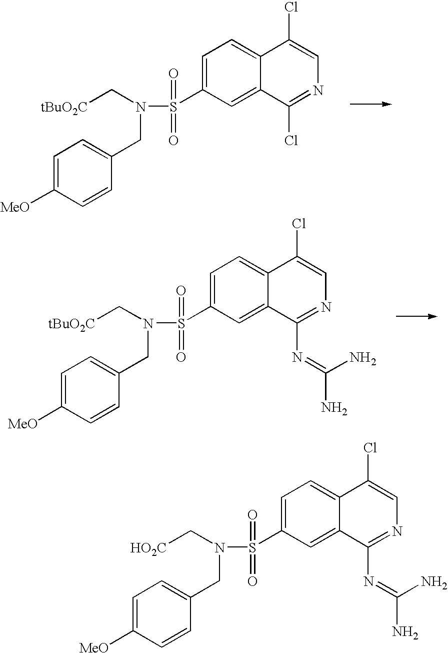

Guanidine hydrochloride (118 mg, 1.24 mmol) was added in one portion to a stirred suspension of NaH (23 mg, 80% dispersion by wt in mineral oil, 0.78 mmol) in DME (10 mL) and the mixture was heated at 60° C. under N₂ for 30 min. N-[(1,4-Dichloro-7-isoquinolinyl)sulphonyl]-N-(4-methoxybenzyl)glycine t-butyl ester (155 mg, 0.31 mmol) was added and the mixture heated at 90° C. for 1 h. The cooled mixture was poured into water and extracted with EtOAc (×3). The combined organic extracts were then washed with water, brine, dried (Na₂SO₄) and evaporated in vacuo. The residue was purified by column chromatography upon silica gel using hexane-EtOAc (80:20), and then CH₂Cl₂-MeOH-0.880NH₃ (95:5:0.5 to 90:10:1) as eluant to give a yellow gum. Trituration with i-Pr₂O gave N-[(4-chloro-1-guanidino-7-isoquinolinyl)sulphonyl]-N-(4-methoxybenzyl)glycine t-butyl ester (80 mg, 0.15 mmol) as a sticky yellow solid. A small sample (10-15 mg) was dissolved in EtOAc, a solution of HCl in Et₂O was added and the solvents were evaporated in vacuo, to give N-[(4-chloro-1-guanidino-7-isoquinolinyl)sulphonyl]-N-(4-methoxybenzyl)glycine t-butyl ester hydrochloride (18 mg) as a solid. (All characterisation data is for the HCl salt).

mp>192° C. (dec).

¹H (DMSO-d₆, 400 MHz) δ 1.2 (9H, s), 3.7 (3H, s), 4.0 (2H, s), 4.4 (2H, s), 6.8 (2H, d), 7.1 (2H, d), 8.3 (1H, d), 8.3 (1H, d), 8.4-8.9 (4H, br s), 8.5 (s, 1H), 8.2 (1H, s) ppm.

LRMS 534, 536 (MH⁺).

Anal. Found: C, 51.36; H, 5.53; N, 11.23. Calc for C₂₄H₂₈ClN₅O₅S.1.0HCl.0.28i-Pr₂O: C, 51.48; H, 5.54; N, 11.69.

N-[(4-chloro-1-guanidino-7-isoquinolinyl)sulphonyl]-N-(4-methoxybenzyl)glycine t-butyl ester (65 mg, 0.122 mmol) was dissolved in CF₃CO₂H (1.0 mL) and the mixture stirred at room temperature for 40 min. The mixture was diluted with PhMe, concentrated in vacuo, and the residue purified by column chromatography upon silica gel using CH₂Cl₂-MeOH-0.880NH₃ (83:15:3) as eluant to give N-[(4-chloro-1-guanidino-7-isoquinolinyl)sulphonyl)-N-(4-methoxybenzyl)glycine (11 mg, 0.023 mmol) as a white solid.

mp>293° C. (dec).

¹H (DMSO-d₆, 400 MHz) δ 3.7 (3H, s), 3.8 (2H, s), 4.4 (2H, s), 6.85 (2H, d), 7.15 (2H, d), 7.2-7.5 (4H, br s), 8.0 (1H, d), 8.1 (1H, d), 8.15 (s, 1H), 9.1 (1H, s) ppm.

Anal. Found: C, 48.44; H, 4.47; N, 14.12. Calc for C₂₀H₂₀ClN₅O₅S.1.0H₂O: C, 48.34; H, 4.27; N, 14.28.

### Example 16

### (a) N-[(4-chloro-1-guanidino-7-isoquinolinyl)sulphonyl]-N2-pyridylmethyl)glycine t-butyl ester

### (b) N-[(4-Chloro-1-guanidino-7-isoquinolinyl)sulphonyl]-N-(2-pyridylmethyl)glycine dihydrochloride

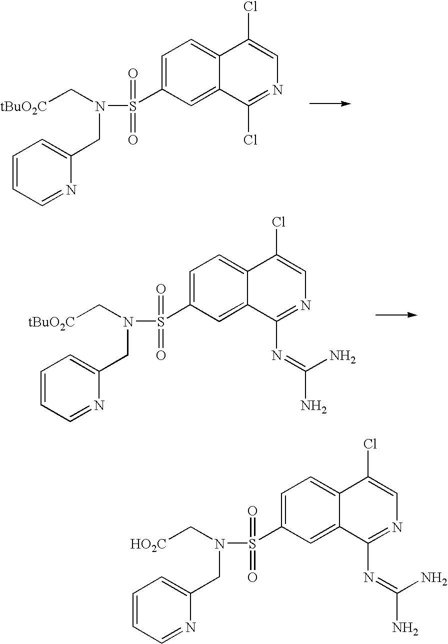

Guanidine hydrochloride (293 mg, 3.07 mmol was added in one portion to a stirred suspension of NaH (57 mg, 80% dispersion by wt in mineral oil, 1.92 mmol) in DME (10 mL) and the mixture was heated at 60° C. under N₂ for 30 min. A solution of N-[(1,4-dichloro-7-isoquinolinyl)sulphonyl]-N-(2-pyridylmethyl)glycine t-butyl ester (370 mg, 0.78 mmol) in DME (10 mL) was added and the mixture heated at 90° C. for 1 h. The solvents were evaporated in vacuo, the residue was dissolved with EtOAc (200 mL), and washed with aqueous NH₄Cl (150 mL), dried (MgSO₄) and evaporated in vacuo. The residue was purified by column chromatography upon silica gel using pentane-EtOAc (100:0 to 20:80) as eluant to give N-[(4-chloro-1-guanidino-7-isoquinolinyl)sulphonyl]-N-(2-pyridylmethyl)glycine t-butyl ester (120 mg, 0.24 mmol) as a pale yellow foam.

¹H (CDCl₃, 400 MHz) δ 1.3 (9H, s), 4.1 (2H, s), 4.65 (2H, s), 7.2 (1H, m), 7.5 (1H, d), 7.65 (1H, dd), 8.05 (1H, d), 8.1 (1H, d), 8.1 (1H, s), 8.45 (1H, d), 9.25 (1H, s) ppm.

LRMS 505 (MH⁺).

Anal. Found: C, 51.93; H, 5.03; N, 15.45. Calc for C₂₂H₂₅ClN₆O₄S.0.1H₂O.0.2EtOAc: C, 52.24; H, 5.18; N, 15.89.

A solution of HCl (3 mL, 2 M, 6 mmol) was added to a solution of N-[(4-chloro-1-guanidino-7-isoquinolinyl)sulphonyl]-N-(2-pyridylmethyl)glycine t-butyl ester (115 mg, 0.23 mmol) in dioxane (5.0 mL) and the mixture was heated at 60° C. for 18 h. The solvents were evaporated in vacuo and the residue triturated with hot EtOAc to give N-((4-chloro-1-guanidino-7-isoquinolinyl)sulphonyl]-N-(2-pyridylmethyl)glycine dihydrochloride (95 mg, 0.167 mmol) as an off-white solid.

mp 216-220° C.

¹H (CD₃OD, 400 MHz) δ 4.4 (2H, s), 5.1 (2H, s), 8.05 (1H, m), 8.3 (1H, d), 8.4 (1H, d), 8.5 (1H, s), 8.5 (1H, d), 8.6 (1H, dd), 8.85 (1H, d), 9.3 (1H, s) ppm.

Anal. Found: C, 39.01; H, 4.01; N, 14.14. Calc for C₁₈H₁₇ClN₆O₄S.2.0HCl.2.0H₂O.0.12dioxane: C, 39.05; H, 4.25; N, 14.78.

### Example 17

### (a) N-[(4-chloro-1guanidino-7-isoquinolinyl)sulphonyl]-N-(3-pyridylmethyl)glycine t-butyl ester

### (b) N-[(4-Chloro-1-guanidino-7-isoquinolinyl)sulphonyl]-N3-pyridylmethyl)glycine dihydrochloride

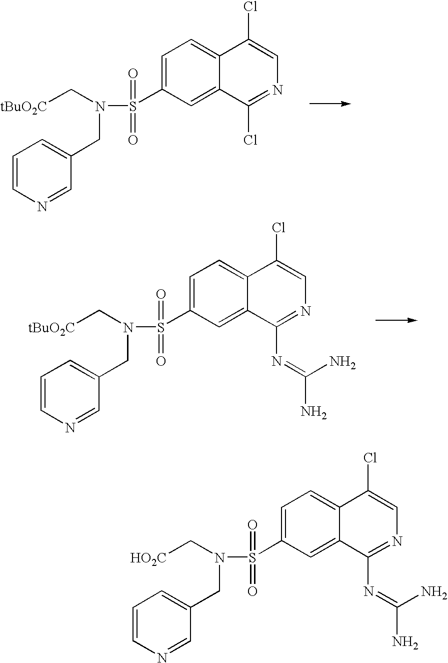

Guanidine hydrochloride (317 mg, 3.32 mmol was added in one portion to a stirred suspension of NaH (62.3 mg, 80% dispersion by wt in mineral oil, 2.08 mmol) in DME (10 mL) and the mixture was heated at 60° C. under N₂ for 30 min. A solution of N-[(I,4-dichloro-7-isoquinolinyl)sulphonyl]-N-(3-pyridylmethyl)glycine t-butyl ester (400 mg, 0.83 mmol) in DME (10 mL) was added and the mixture heated at 80° C. for 4 h. The solvents were evaporated in vacuo, the residue was dissolved with EtOAc (200 mL), and washed with aqueous NH₄Cl (200 mL), dried (MgSO₄) and evaporated in vacuo. The residue was purified by column chromatography upon silica gel using (i) pentane-EtOAc (70:30 to 50:50) and then (ii) CH₂Cl₂-MeOH-0.880NH₃ (95:5:0.5 to 90:101) as eluant to give N-[(4-chloro-1-guanidino-7-isoquinolinyl)sulphonyl]-N-(3-pyridylmethyl)glycine t-butyl ester (104 mg, 0.21 mmol) as a pale yellow solid.

¹H (CDCl₃, 400 MHz) δ 1.3 (9H, s), 3.8 (2H, s), 4.5 (2H, s), 6.4-6.8 (4H, br), 7.2 (1H, m), 7.6 (1H, d), 8.0 (1H, d), 8.05 (1H, s), 8.05 (1H, d), 8.4 (1H, s), 8.5 (1H, d), 9.3 (1H, s) ppm.

LRMS 505, 507 (MH⁺).

Anal. Found: C, 51.95; H, 5.02; N, 16.25. Calc for C₂₂H₂₅ClN₆O₄S: C, 52.33; H, 4.99; N, 16.44.

CF₃CO₂H (1.0 mL) was added to a stirred solution of N-[(4-chloro-1-guanidino-7-isoquinolinyl)sulphonyl]-N-(3-pyridylmethyl)glycine t-butyl ester (100 mg, 0.20 mmol) in CH₂Cl₂ (1.0 mL) and the mixture was stirred at 23° C. for 3.5 h. The solvents were evaporated in vacuo, azeotroping with PhMe and CH₂Cl₂. The oily residue was dissolved in EtOAc and a solution of EtOAc saturated with HCl (3.0 mL) was added which gave a precipitate. The white solid was collected by filtration and dried to give N-[(4-chloro-1-guanidino-7-isoquinolinyl)sulphonyl]-N-(3-pyridylmethyl)glycine dihydrochloride (48 mg, 0.086 mmol).

¹H (CD₃OD, 400 MHz) δ 4.25 (2H, s), 4.9 (2H, s), 8.05 (1H, dd), 8.4 (1H, d), 8.45 (1H, s), 8.5 (1H, d), 8.7 (1H, d), 8.8 (1H, d), 9.0 (1H, s), 9.2 (1H, s) ppm.

Anal. Found: C, 39.32; H, 4.07; N, 15.07. Calc for C₁₈H₁₇ClN₆O₄S.2.0HCl.1.5H₂O.0.05EtOAc.0.05 CH₂Cl₂: C, 39.19; H, 3.72; N, 14.64.

### Example 18

### (a) N-[(4-chloro-1-guanidino-7-isoquinolinyl)sulphonyl]-N-(4-pyridylmethyl)glycine t-butyl ester

### (b) N-1(4-Chloro-1-guanidino-7-isoquinolinyl)sulphonyl]-N-(4-pyridylmethyl)glycine dihydrochloride

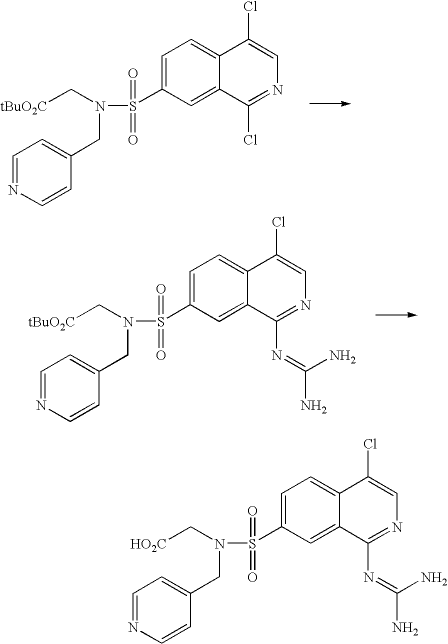

Guanidine hydrochloride (300 mg, 3.14 mmol was added in one portion to a stirred suspension of NaH (59 mg, 80% dispersion by wt in mineral oil, 1.97 mmol) in DME (10 mL) and the mixture was heated at 60° C. under N₂ for 30 min. A solution of N-[(I,4-dichloro-7-isoquinolinyl)sulphonyl]-N-(4-pyridylmethyl)glycine t-butyl ester (379 mg, 0.79 mmol) in DME (10 mL) was added and the mixture heated at 80° C. for 4 h. The solvents were evaporated in vacuo, the residue was dissolved with EtOAc (200 mL), and washed with aqueous NH₄Cl (150 mL), dried (MgSO₄) and evaporated in vacuo. The residue was purified by repeated column chromatography upon silica gel using (i) pentane-EtOAc (70:30 to 50:50) and then with (ii) CH₂Cl₂-MeOH-0.880NH₃ (95:5:0.5 to 90:101) as eluant to give N-[(4-chloro-1-guanidino-7-isoquinolinyl)sulphonyl]-N-(4-pyridylmethyl)glycine t-butyl ester (96 mg, 0.19 mmol).

¹H (CDCl₃, 400 MHz) δ 1.3 (9H, s), 3.9 (2H, s), 4.55 (2H, s), 7.25 (2H, d), 8.05 (1H, d), 8.1 (1H, d), 8.15 (1H, s), 8.6 (2H, d), 9.3 (1H, s) ppm.

LRMS 505, 507 (MH⁺).

Anal. Found: C, 52.63; H, 5.09; N, 16.18. Calc for C₂₂H₂₅ClN₆O₄S: C, 52.33; H, 4.99; N, 16.64.

CF₃CO₂H (1.0 mL) was added to a stirred solution of N-[(4-chloro-1-guanidino-7-isoquinolinyl)sulphonyl]-N-(4-pyridylmethyl)glycine t-butyl ester (88 mg, 0.17 mmol) in CH₂Cl₂ (1.0 mL) and the mixture was stirred at 23° C. for 3.5 h. The solvents were evaporated in vacuo, azeotroping with CH₂Cl₂. The oily residue was dissolved in CH₂Cl₂-MeOH (1.0 mL, 9:1) and a solution of EtOAc saturated with HCl (3.0 mL) was added which gave a precipitate. The white solid was collected by filtration and dried to give N-[(4-chloro-1-guanidino-7-isoquinolinyl)sulphonyl]-N-(4-pyridylmethyl)glycine dihydrochloride (18 mg, 0.033 mmol). ¹H (CD₃OD, 400 MHz) δ 4.3 (2H, s), 5.0 (2H, s), 8.2 (2H, d), 8.4 (1H, d), 8.5 (1H, s), 8.55 (1H, d), 8.8 (2H, d), 9.1 (1H, s) ppm.

Anal. Found: C, 39.57; H, 4.12; N, 14.85. Calc for C₁₈H₁₇ClN₆O₄S.2.0HCl.1.5H₂O: C, 39.39; H, 4.04; N, 15.39.

### Example 19

### (a) N-[(4-chloro-1-guanidino-7-isoquinolinyl)sulphonyl]-N-[(1R)-1-phenylethyl]glycine t-butyl ester

### (b) N-[(4-Chloro-1-guanidino-7-isoquinolinyl)sulphonyl]-N-[(1R)-1-phenylethyl]glycine hydrochloride

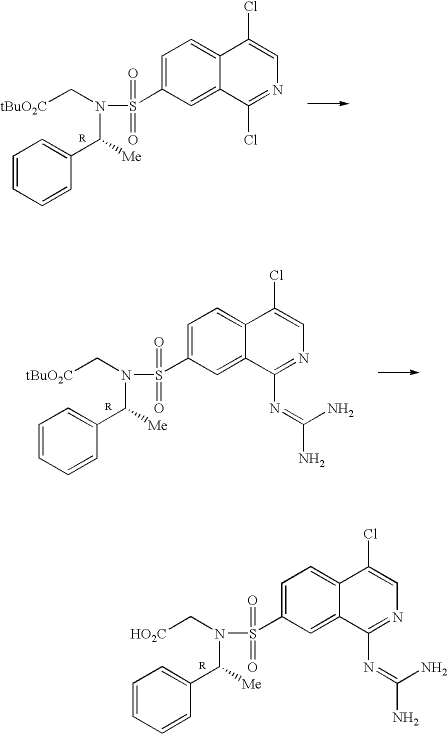

NaH (30 mg, 80% dispersion by wt in mineral oil, 1.01 mmol) was added in one portion to a stirred suspension of guanidine hydrochloride (154 mg, 1.61 mmol) in DME (6.0 mL) and the mixture was heated at 60° C. under N₂ for 30 min. A solution of N-[(1,4-dichloro-7-isoquinolinyl)sulphonyl]-N-[(1R)-1-phenylethyl]glycine t-butyl ester (200 mg, 0.40 mmol) in DME (3.0 mL) was added and the mixture heated at 95° C. for 5 h. The solvents were evaporated in vacuo and the residue was purified by column chromatography upon silica gel using pentane-EtOAc (50:50 to 33:66) as eluant to give N-[(4-chloro-1-guanidino-7-isoquinolinyl)sulphonyl]-N-[(1R)-1-phenylethyl]glycine t-butyl ester (125 mg, 0.23 mmol) as pale yellow foam after repeated evaporation from CH₂Cl₂.

mp 106-111° C.

¹H (DMSO-d₆, 300 MHz) δ 1.2 (9H, s), 1.3 (3H, d), 3.7 (1H, d), 3.95 (1H, d), 5.05 (1H, q), 7.1-7.4 (4H, br), 7.2-7.3 (5H, m), 8.0 (1H, d), 8.1 (1H, s), 8.2 (1H, d), 9.15 (1H, s) ppm.

LRMS 518, 520 (MH⁺), 1035 (M₂H⁺).

Anal. Found: C, 55.15; H, 5.55; N, 12.84. Calc for C₂₄H₂₈ClN₅O₄S.0.2EtOAc.0.1CH₂Cl₂: C, 54.96; H, 5.52; N, 12.87.

N-[(4-Chloro-1-guanidino-7-isoquinolinyl)sulphonyl]-N-[(1R)-1-phenylethyl]glycine t-butyl ester (100 mg, 0.19 mmol) was dissolved in a solution of EtOAc saturated with HCl (7.0 mL) and the mixture stirred at room temperature for 4 h. The mixture was concentrated in vacuo and the residue triturated with EtOAc to give N-[(4-Chloro-1-guanidino-7-isoquinolinyl)sulphonyl]-N-[(1R)-1-phenylethyl]glycine hydrochloride (75 mg, 0.14 mmol) as a white powder.

mp 185-190° C.

¹H (DMSO-d₆, 300 MHz) δ 1.35 (3H, d), 3.85 (1H, d), 4.15 (1H, d), 5.3 (1H, q), 7.15 (5H, br s), 8.3 (1H, d), 8.4-8.8 (4H, br), 8.4 (1H, d), 8.5 (1H, s), 9.1 (1H, s), 11.3 (1H, br), 12.5 (1H, br) ppm.

Anal. Found: C, 47.42; H, 4.40; N, 13.54. Calc for C₂₀H₂₀ClN₅O₄S.1.0HCl.0.5H₂O.0.2EtOAc: C, 47.59; H, 4.53; N, 13.34.

### Example 20

### (a) N-[(4-chloro-1-guanidino7-isoquinolinyl)sulphonyl]-N-[(1S)-1-phenylethyl]glycine t-butyl ester

### (b) N-[(4-Chloro-1-guanidino-7-isoquinolinyl)sulphonyl]-N-[(1S)-1-phenylethyl]glycine hydrochloride

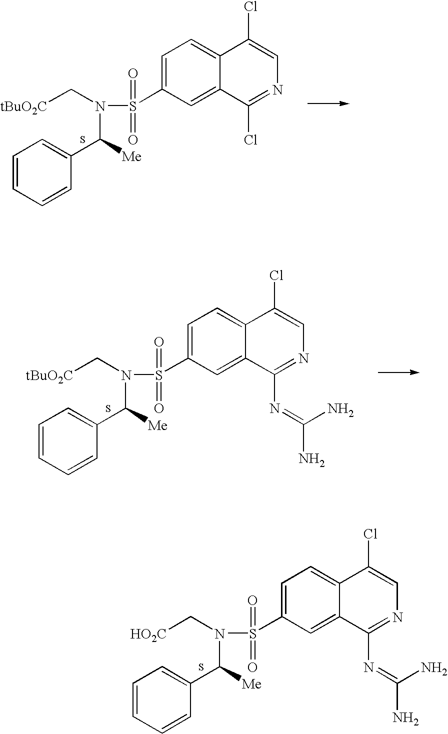

NaH (30 mg, 80% dispersion by wt in mineral oil, 1.01 mmol) was added in one portion to a stirred suspension of guanidine hydrochloride (154 mg, 1.61 mmol) in DME (6.0 mL) and the mixture was heated at 60° C. under N₂ for 30 min. A solution of N-[(1,4-dichloro-7-isoquinolinyl)sulphonyl]-N-[(1S)-1-phenylethyl]glycine t-butyl ester (200 mg, 0.40 mmol) in DME (3.0 mL) was added and the mixture heated at 95° C. for 5 h. The solvents were evaporated in vacuo and the residue was purified by column chromatography upon silica gel using pentane-EtOAc (50:50 to 33:66) as eluant to give N-[(4-chloro-1-guanidino-7-isoquinolinyl)sulphonyl]-N-[(1S)-1-phenylethyl]glycine t-butyl ester (128 mg, 0.23 mmol) as pale yellow foam after repeated evaporation from CH₂Cl₂.

mp 109-115° C.

¹H (DMSO-d₆, 300 MHz) δ 1.2 (9H, s), 1.3 (3H, d), 3.7 (1H, d), 3.95 (1H, d), 5.05 (1H, q), 7.1-7.45 (4H, br), 7.2-7.3 (5H, m), 8.0 (1H, d), 8.1 (1H, s), 8.2 (1H, d), 9.15 (1H, s) ppm.

LRMS 518, 520 (MH⁺), 1035 (M₂H⁺).

Anal. Found: C, 55.26; H, 5.56; N, 12.86. Calc for C₂₄H₂₈ClN₅O₄S.0.1EtOAc.0.05CH₂Cl₂: C, 55.28; H, 5.54; N, 12.97.

N-[(4-Chloro-1-guanidino-7-isoquinolinyl)sulphonyl]-N-[(1S)-1-phenylethyl]glycine t-butyl ester (100 mg, 0.19 mmol) was dissolved in a solution of EtOAc saturated with HCl (4.0 mL) and the mixture stirred at room temperature for 4 h. The mixture was concentrated in vacuo and the residue triturated with EtOAc to give N-[(4-Chloro-1-guanidino-7-isoquinolinyl)sulphonyl]-N-[(1S)-1-phenylethyl]glycine hydrochloride (72 mg, 0.14 mmol) as a white powder.

mp 196-200° C.

¹H (DMSO-d₆, 300 MHz) δ 1.35 (3H, d), 3.85 (1H, d), 4.15 (1H, d), 5.3 (1H, q), 7.15 (5H, br s), 8.3 (1H, d), 8.4-8.8 (4H, br), 8.4 (1H, d), 8.5 (1H, s), 9.1 (1H, s), 11.3 (1H, br), 12.4(1H, br) ppm.

Anal. Found: C, 47.42; H, 4.30; N, 13.51. Calc for C₂₀H₂₀ClN₅O₄S.1.0HCl.1.0H₂O.0.1EtOAc: C, 47.47; H, 4.45; N, 13.57.

### Example 21

### (a) N-benzyl-N-[(4-chloro-1-guanidino-7-isoquinolinyl)sulphonyl]-L-alanine t-butyl ester

### (b) N-Benzyl-N-[(4-chloro-1-guanidino-7-isoquinolinyl)sulphonyl]-L-alanine hydrochloride

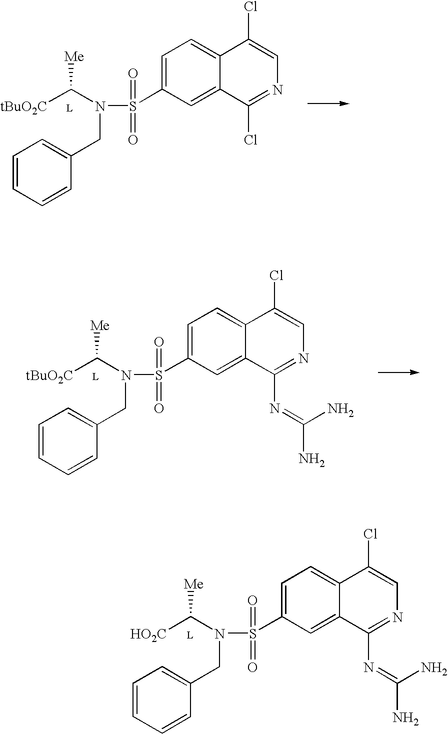

NaH (30 mg, 80% dispersion by wt in mineral oil, 1.01 mmol) was added in one portion to a stirred suspension of guanidine hydrochloride (154 mg, 1.61 mmol) in DME (5.0 mL) and the mixture was heated at 60° C. under N₂ for 45 min. A solution of N-benzyl-N-[(1,4-dichloro-7-isoquinolinyl)sulphonyl]-L-alanine t-butyl ester (200 mg, 0.40 mmol) in DME (2.0 mL) was added and the mixture heated at 95° C. for 4 h. The solvents were evaporated in vacuo and the residue was purified by column chromatography upon silica gel using pentane-EtOAc (50:50 to 20:80) as eluant to give N-benzyl-N-[(4-chloro-1-guanidino-7-isoquinolinyl)sulphonyl]-L-alanine t-butyl ester (120 mg, 0.225 mmol) as pale yellow foam after repeated evaporation from CH₂Cl₂,

¹H (DMSO-d₆, 300 MHz) δ 1.1 (9H, s), 1.15 (3H, d), 4.35 (1H, d), 4.5 (1H, q), 4.7 (1H, d), 7.1-7.45 (4H, br), 7.2-7.4 (5H, m), 8.0 (1H, d), 8.1 (1H, d), 8.15 (1H, s), 9.1 (1H, s) ppm.

LRMS 518, 520 (MH⁺).

Anal. Found: C, 55.33; H, 5.55; N, 12.82. Calc for C₂₄H₂₈ClN₅O₄S.0.1EtOAc.0.05CH₂Cl₂: C, 55.30; H, 5.48; N, 13.19.

N-Benzyl-N-[(4-chloro-1-guanidino-7-isoquinolinyl)sulphonyl]-L-alanine 1-butyl ester (100 mg, 0.19 mmol) was dissolved in a solution of EtOAc saturated with HCl (5.0 mL) and the mixture stirred at room temperature for 18 h. The mixture was concentrated in vacuo, azeotroping with EtOAc, to give N-benzyl-N-[(4-chloro-1-guanidino-7-isoquinolinyl)sulphonyl]-L-alanine hydrochloride (77 mg, 0.15 mmol) as a white powder.

mp 256-262° C.

¹H (DMSO-d₆, 300 MHz) δ 1.2 (3H, d), 4.35 (1H, d), 4.7 (1H, q), 4.8 (1H, d), 7.1-7.4 (5H, m), 8.3 (2H, s), 8.4-8.7 (4H, br), 8.5 (1H, s), 9.05 (1H, s), 11.2 (1H, br), 12.7 (1H, br) ppm.

LRMS 461, 463 (MH⁺).

Anal. Found: C, 48.02; H, 4.38; N, 13.33. Calc for C₂₀H₂₀ClN₅O₄S.1.0HCl.0.25H₂O.0.1EtOAc: C, 47.88; H, 4.39; N, 13.69.

### Example 22

### (a) N-(t-butoxycarbonylmethyl)-N-[(4-chloro-1-guanidino-7-isoquinolinyl)sulphonyl]glycine t-butyl ester

### (b) N-(Carboxymethyl)-N-[(4-chloro-1-guanidino-7-isoquinolinyl)sulphonyl]glycine hydrochloride

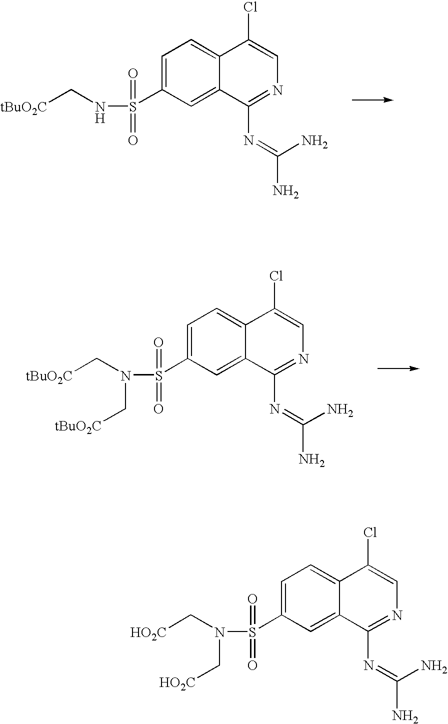

Anhydrous K₂CO₃ (88 mg, 0.64 mmol) and then t-butyl bromoacetate (56 μL, 0.38 mmol) were added to a stirred solution of N[(4-chloro-1-guanidino-7-isoquinolinyl)sulphonyl]glycine t-butyl ester (132 mg, 0.33 mmol) in DMF (2.0 mL) and the mixture was stirred at 23° C. for 18 h. The mixture was diluted with EtOAc (300 mL), washed with brine (150 mL), water (200 mL), dried (MgSO₄) and evaporated in vacuo. The residue was purified by column chromatography upon silica gel using pentane-EtOAc (80:20 to 50:50) as eluant to give N-(t-butoxycarbonylmethyl)-N-[(4-chloro-1-guanidino-7-isoquinolinyl)sulphonyl]glycine t-butyl ester (101 mg, 0.19 mmol) as a pale yellow foam.

¹H (CDCl₃, 400 MHz) δ 1.4 (18H, s), 4.1 (4H, s), 8.0 (1H, d), 8.1 (1H, d), 8.15 (1H, s), 9.25 (1H, s) ppm.

LRMS 528 (MH⁺).

Anal. Found: C, 49.57; H, 5.78; N, 12.73. Calc for C₂₂H₃₀ClN₅O₆S.0.1H₂O.0.1EtOAc: C, 49.95; H, 5.80; N, 13.00.

A solution of HCl (3 mL, 2 M, 6 mmol) was added to a solution of N-[(4-chloro-1-guanidino-7-isoquinolinyl)sulphonyl]-N-(t-butoxycarbonylmethyl)glycine t-butyl ester (90 mg, 0.17 mmol) in dioxane (4.0 mL). The mixture was stirred at 23° C. for 18 h and then heated at 70° C. The solvents were evaporated in vacuo and the residue dried to give N-(carboxymethyl)-N-[(4-chloro-1-guanidino-7-isoquinolinyl)sulphonyl]glycine hydrochloride (61 mg, 0.127 mmol) as a white solid.

mp 296-300° C. (dec).

¹H (DMSO-d₆, 400 MHz) δ 4.05 (4H, s), 7.9-8.3 (4H, br), 8.2 (1H, d), 8.25 (1H, d), 8.35 (1H, s), 9.0 (1H, s) ppm.

Anal. Found: C, 38.29; H, 3.58; N, 14.13. Calc for C₁₄H₁₄ClN₅O₆S.1.0HCl.0.1H₂O.0.3dioxane: C, 37.99; H, 3.69; N, 14.57.

### Example 23

### (a) N-[(4-chloro-1-guanidino-7-isoquinolinyl)sulphonyl]-L-alanine 1-butyl ester

### (b) N-[(4-Chloro-1-guanidino-7-isoquinolinyl)sulphonyl]-L-alanine trifluoroacetate

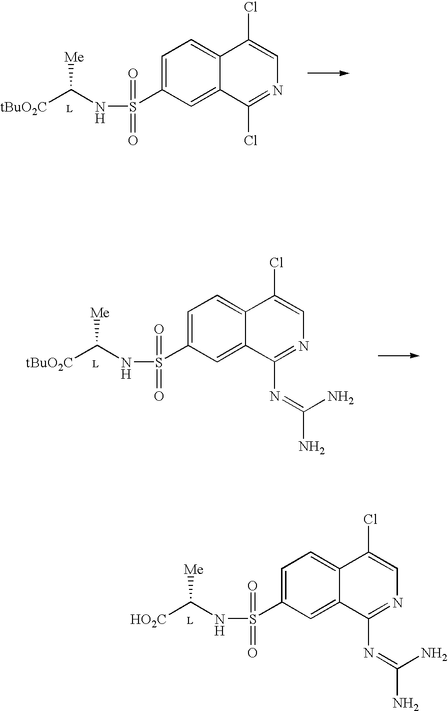

NaH (37 mg, 80% dispersion by wt in mineral oil, 1.23 mmol) was added in one portion to a stirred solution of guanidine hydrochloride (189 mg, 1.97 mmol) in DME (6 mL) and the mixture was heated at 60° C. under N₂ for 30 min. 1-{[(1,4-Dichloro-7-isoquinolinyl)sulphonyl]amino}-L-alanine t-butyl ester (200 mg, 0.49 mmol) was added and the mixture heated at 90° C. for 7 h. The cooled mixture was concentrated in vacuo, the residue suspended in water and extracted with EtOAc (3×30 mL). The combined organic extracts were dried (MgSO₄) and the solvents evaporated in vacuo. The residue was purified by column chromatography upon silica gel using CH₂Cl₂-MeOH-0.880NH₃ (95:5:0.5) as eluant to give N-[(4-chloro-1-guanidino-7-isoquinolinyl)sulphonyl]-L-alanine t-butyl ester (160 mg, 0.37 mmol) as a white powder.

¹H (DMSO-d₆, 300 MHz) δ 1.1 (9H, s), 1.15 (3H, d), 3.8 (1H, dq), 7.1-7.4 (4H, br), 8.0 (1H, d), 8.05 (1H, d), 8.1 (1H, s), 8.3 (1H, d), 9.05 (1H, s) ppm.

CF₃CO₂H (1.0 mL) was added to a stirred solution of N-[(4-chloro-1-guanidino-7-isoquinolinyl)sulphonyl]-L-alanine t-butyl ester (ca. 150 mg, 0.35 mmol) in CH₂Cl₂ (3.0 mL) and the mixture stirred at room temperature for 2 h. The mixture was evaporated in vacuo, azeotroping with PhMe and CH₂Cl₂, and then triturated with Et₂O to give N-[(4-chloro-1-guanidino-7-isoquinolinyl)sulphonyl]-L-alanine trifluoroacetate (62 mg, 0.126 mmol) as a white powder.

mp>250° C.

¹H (CD₃OD+TFA-d, 300 MHz) δ 1.35 (3H, d), 4.05 (1H, q), 8.3 (1H, d), 8.4 (1H, s), 8.45 (1H, d), 8.9 (1H, s) ppm.

LRMS 389, 391 (MNH₄⁺).

Anal. Found: C, 36.66; H, 3.11; N, 14.00. Calc for C₁₃H₁₄ClN₅O₄S.1.0CF₃CO₂H.0.3H₂O: C, 36.64; H, 3.21; N, 14.24.

### Example 24

### (a) N-[(4-chloro-1-guanidino-7-isoquinolinyl)sulphonyl]-D-alanine methyl ester

### (b) N-[(4-chloro-1-guanidino-7-isoquinolinyl)sulphonyl]-D-alanine hydrochloride

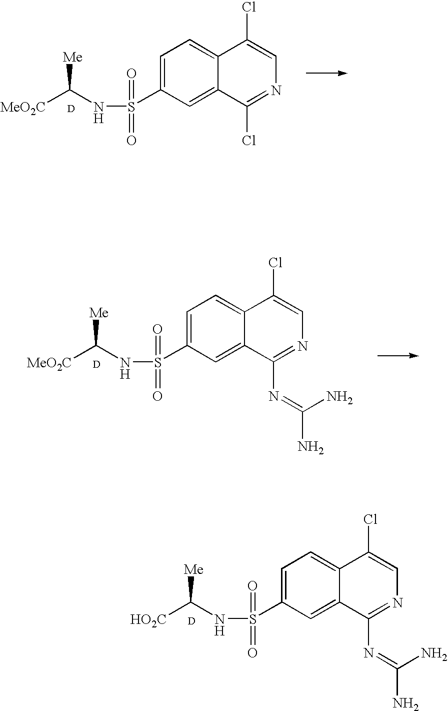

NaH (35 mg, 80% dispersion by wt in mineral oil, 1.17 mmol) was added in one portion to a stirred solution of guanidine hydrochloride (179 mg, 1.87 mmol) in DMSO (5 mL) and the mixture was heated at 60° C. under N₂ for 45 min. 1-{[(1,4-Dichloro-7-isoquinolinyl)sulphonyl]amino}-D-alanine methyl ester (170 mg, 0.47 mmol) was added and the mixture heated at 90° C. for 4 h. The cooled mixture was poured into water and extracted with EtOAc (3×30 mL). The combined organic extracts were dried (MgSO₄) and the solvents evaporated in vacuo. The residue was purified by column chromatography upon silica gel using pentane-EtOAc (66:33 to 0:100) as eluant to give N-[(4-chloro-1-guanidino-7-isoquinolinyl)sulphonyl]-D-alanine methyl ester (22 mg, 0.057 mmol) as a yellow foam/oil.

¹H (CD₃OD, 300 MHz) δ 1.3 (3H, d), 3.4 (3H, s),4.1 (1H, q), 8.1 (1H, d), 8.1 (1H, d), 8.15 (1H, s), 9.1 (1H, s) ppm.

LRMS 386, 388 (MH⁺).

A solution of NaOH (1 mL, 2 M, 2 mmol) was added to a solution of N-[(4-chloro-1-guanidino-7-isoquinolinyl)sulphonyl]-D-alanine methyl ester (17 mg, 0.044 mmol) in MeOH (3 mL) and the mixture was heated at 60° C. for 18 h. The cooled mixture was neutrilised with dilute HCl (2 M), the MeOH was evaporated in vacuo, and the residue triturated with water (10 mL). The solid was collected by filtration, with water washing, and dried under high vacuum to give N-[(4-chloro-1-guanidino-7-isoquinolinyl)sulphonyl]-D-alanine hydrochloride (9 mg, 0.021 mmol) as an off-white powder.

¹H (DMSO-d₆, 300 MHz) δ 1.2 (3H, d), 3.8 (1H, dq), 7.2-7.6 (4H, br), 8.05 (1H, d), 8.1 (1H, d), 8.15 (1H, s), 8.2 (1H, m), 9.1 (1H, s) ppm.

Anal. Found: C, 37.56; H, 3.98; N, 15.74. Calc for C₁₃H₁₄ClN₅O₄S.1.0HCl.0.5H₂O: C, 37.42; H, 3.86; N, 16.78.

### Example 25

### (a) 1-{[(4-chloro-1-guanidino-7-isoquinolinyl)sulphonyl]amino}-L-valine t-butyl ester

### (b) N-[(4-Chloro-1-guanidino-7-isoquinolinyl)sulphonyl]-L-valine trifluoroacetate

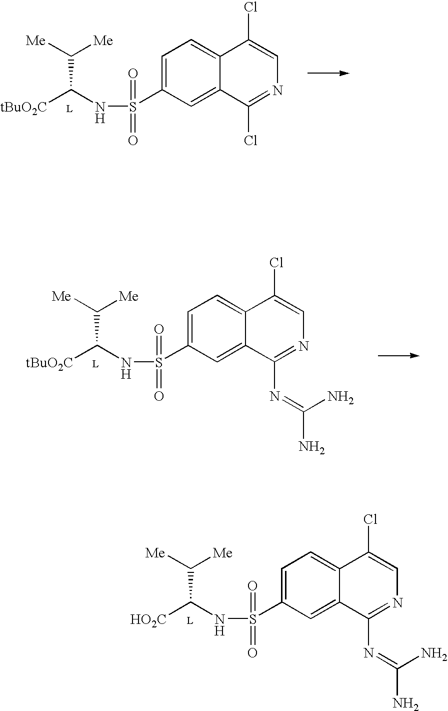

NaH (35 mg, 80% dispersion by wt in mineral oil, 1.17 mmol) was added in one portion to a stirred solution of guanidine hydrochloride (176 mg, 1.84 mmol) in DMA (4 mL) under N₂ and the mixture was heated at 60° C. for 30 min. 1-{[(1,4-Dichloro-7-isoquinolinyl)sulphonyl]amino}-L-valine t-butyl ester (161 mg, 0.43 mmol) was added in one portion and the mixture heated at 80° C. for 18 h. The cooled mixture was poured into water (50 mL), extracted with EtOAc (2×20 mL) and the combined organic extracts were washed with brine, dried (Na₂SO₄) and evaporated in vacuo. The residue was dissolved Et₂O and a solution of HCl in Et₂O (1 M) was added which gave a white precipitate. The Et₂O was decanted and the solid residue dissolved in MeCN and the solution cooled to ca. 0° C. which gave a precipate. This solid was collected by filtration and then dried to give 1-{[(4-chloro-1-guanidino-7-isoquinolinyl)sulphonyl]amino}-L-valine t-butyl ester hydrochloride (36 mg, 0.072 mmol) as a white solid. Evaporation of the combined organic mother liquors gave a gum which was purified by column chromatography upon silica gel using CH₂Cl₂-MeOH-0.880NH₃ (90:10:1) as eluant to give 1-{[(4-chloro-1-guanidino-7-isoquinolinyl)sulphonyl]amino}-L-valine t-butyl ester (104 mg, 0.228 mmol). (The sample was characterised as the hydrochloride salt.)

mp 192-194° C. (dec).

¹H (DMSO-d₆, 300 MHz) δ 0.8 (3H, d), 0.85 (3H, d), 1.05 (9H, s), 2.0 (1H, sept), 3.7 (1H, dd), 8.3 (1H, d), 8.4 (1H, d), 8.4 (1H, d), 8.45 (1H, s), 8.5-8.7 (4H, br), 9.05 (1H, s), 11.3 (1H, br), ppm.

LRMS 456, 458 (MH⁺).

Anal. Found: C, 45.67; H, 5.54; N, 13.97. Calc for C₁₉H₂₆ClN₅O₄S.1.0HCl.0.5H₂O: C, 45.51; H, 5.63; N, 13.97.

1-{[(4-Chloro-1-guanidino-7-isoquinolinyl)sulphonyl]amino}-L-valine t-butyl ester (104 mg, 0.228 mmol) was dissolved in CF₃CO₂H (1.0 mL) and the mixture stirred at room temperature for 1 h. The mixture was diluted with PhMe (25 mL) and concentrated in vacuo. The residue was crystallised with Et₂O containing a small amount of EtOAc to give a white solid. This solid was then triturated with water and dried to give 1-{[(4-chloro-1-guanidino-7-isoquinolinyl)sulphonyl]amino)-L-valine trifluoroacetate (39 mg, 0.084 mmol).

mp>300° C.

¹H (TFA-d, 400 MHz) δ 0.95 (3H, d), 1.0 (3H, d), 2.25 (1H, sept), 4.0 (1H, d), 8.3 (1H, d), 8.4 (1H, s), 8.55 (1H, d), 9.0 (1H, s) ppm.

LRMS 400, 402 (MH⁺).

Anal. Found: C, 41.29; H, 4.37; N, 14.99. Calc for C₁₅H₁₈ClN₅O₄S.0.5CF₃CO₂H.0.3H₂O: C, 41.57; H, 4.16; N, 15.15.

### Example 26

### (a) 1-{[(4-chloro-1-guanidino-7-isoquinolinyl)sulphonyl]amino}-D-valine t-butyl ester hydrochloride

### (b) N-[(4-Chloro-1-guanidino-7-isoquinolinyl)sulphonyl]-D-valine hydrochloride

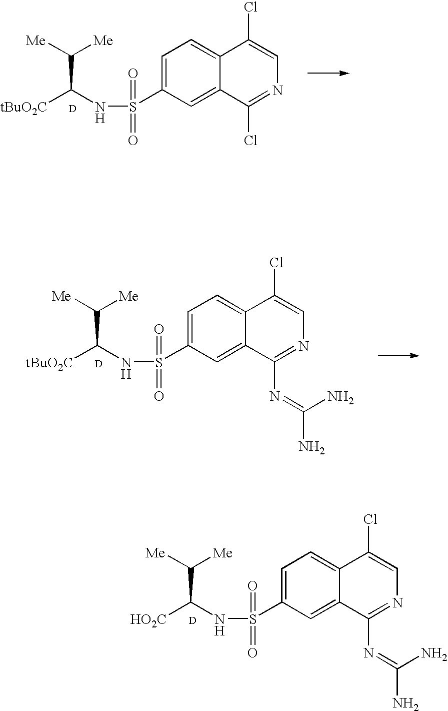

NaH (35 mg, 80% dispersion by wt in mineral oil, 1.17 mmol) was added in one portion to a stirred solution of guanidine hydrochloride (176 mg, 1.84 mmol) in DMSO (2.5 mL) under N₂ and the mixture was heated at 23° C. for 30 min. 1-{[(1.4-Dichloro-7-isoquinolinyl)sulphonyl]amino}-D-valine t-butyl ester (200 mg, 0.46 mmol) was added in one portion and the mixture heated at 90° C. for 3 h. The cooled mixture was poured into water, extracted with EtOAc and the combined organic extracts were washed with brine, dried (MgSO₄) and evaporated in vacuo. The residue was dissolved Et₂O and a solution of HCl in Et₂O (0.5 mL, 1 M) was added which gave a white precipitate. Purification by column chromatography upon silica gel using CH₂Cl₂-MeOH-0.880NH₃ (95:5:0.5) as eluant furnished 1. the product which was again treated with a solution of HCl in Et₂O (1 M) to give 1-{[(4-chloro-1-guanidino-7-isoquinolinyl)sulphonyl]amino}-D-valine t-butyl ester hydrochloride (76.6 mg, 0.151 mmol).

mp 124-125° C. (dec).

¹H (DMSO-d₆, 300 MHz) δ 0.8 (3H, d), 0.85 (3H, d), 1.05 (9H, s), 2.0 (1H, sept), 3.7 (1H, dd), 8.3 (1H, d),8.4 (1H, d), 8.4 (1H, d), 8.45 (1H, s), 8.4-8.8 (4H, br), 9.05 (1H, s), 11.2 (1H, br) ppm.

LRMS 456, 458 (MH⁺), 478, 480 MNa⁺).

Anal. Found: C, 46.07; H, 5.67; N, 13.50. Calc for C₁₉H₂₆ClN₅O₄S.1.0HCl.0.5MeOH: C, 46.07; H, 5.75; N, 13.77.

1{[(4-Chloro-1-guanidino-7-isoquinolinyl)sulphonyl]amino}-D-valine t-butyl ester hydrochloride (61 mg, 0.12 mmol) was dissolved in a solution of EtOAc saturated with HCl (10 mL) at 0° C., and the mixture stirred at room temperature for 4 h. The mixture was concentrated in vacuo, the residue extracted with hot EtOAc, and the organic solution was then concentrated in vacuo and dried to give 1-{[(4-chloro-1-guanidino-7-isoquinolinyl)sulphonyl]amino}-D-valine hydrochloride (24.3 mg, 0.050 mmol) as a pale yellow solid.

mp>190° C. (dec).

¹H (TFA-d, 400 MHz) δ 0.95 (3H, br s), 1.0 (3H, br s), 2.3 (1H, br s), 4.05 (1H, br s), 8.35 (1H, br s), 8.4 (1H, br s), 8.55 (1H, br s), 9.1 (1H, br s) ppm.

LRMS 400 (MH⁺), 417 (MNH₄⁺).

Anal. Found: C, 41.29; H, 4.76; N, 14.16. Calc for C₁₅H₁₈ClN₅O₄S.1.0HCl0.7H₂O.0.4EtOAc: C, 41.18; H, 4.91; N, 14.46.

### Example 27

### (a) 1-{[(4chloro-1-guanidino-7-isoquinolinyl)sulphonyl]amino}-D-tert-leucine t-butyl ester hydrochloride

### (b) N-[(4-Chloro-1-guanidino-7-isoquinolinyl)sulphonyl]-D-tert-leucine hydrochloride

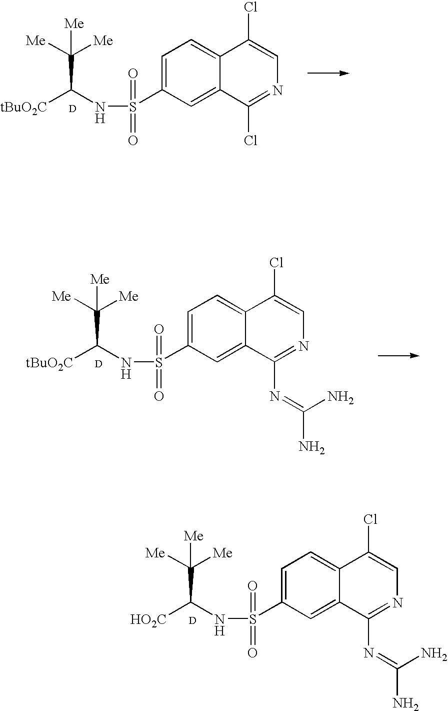

NaH (58 mg, 80% dispersion by wt in mineral oil, 1.27 mmol) was added in one portion to a stirred solution of guanidine hydrochloride (191 mg, 2.0 mmol) in DMSO (5.0 mL) under N₂ and the mixture was heated at 23° C. for 30 min. A solution of 1-{[(1,4-dichloro-7-isoquinolinyl)sulphonyl]amino}-D-tert-leucine t-butyl ester (225 mg, 0.50 mmol) in DMSO (3.0 mL) was added in one portion and the mixture heated at 90° C. for 9 h. A second portion of guanidine (0.67 mmol)[prepared from guanidine hydrochloride (100 mg) and NaH (20 mg)] in DMSO (1.0 mL) was added and the mixture heated at 90° C. for an additional 8 h. The cooled mixture was poured into water, extracted with EtOAc and the combined organic extracts were washed with water, brine, dried (MgSO₄) and evaporated in vacuo. The residue was dissolved Et₂O and a solution of HCl in Et₂O (1.5 mL, 1 M) was added which gave a white precipitate. The solvents were evaporated in vacuo and the residue triturated with Et₂O to give 1-{[(4-chloro-1-guanidino-7-isoquinolinyl)sulphonyl]amino)-D-tert-leucine t-butyl ester hydrochloride (222 mg, 0.43 mmol).

mp 187-189° C.

¹H (DMSO-d₆, 400 MHz) δ 0.9 (9H, s), 0.95 (9H, s), 3.6 (1H, d), 8.3 (1H, d), 8.4 (1H, d), 8.4-8.8 (4H, br), 8.5 (1H, s), 9.0 (1H, s), 11.15 (1H, br) ppm.

LRMS 470, 472 (MH⁺).

Anal. Found: C, 46.55; H, 5.78; N, 13.46. Calc for C₂₀H₂₈ClN₅O₄S.1.0HCl.0.5H₂O: C, 46.60; H, 5.87; N, 13.59.

1-{[(4-Chloro-1-guanidino-7-isoquinolinyl)sulphonyl]amino}-D-tert-leucine t-butyl ester hydrochloride (188 mg, 0.36 mmol) was dissolved in a solution of EtOAc saturated with HCl (30 mL) and the mixture stirred at room temperature for 5 h. The mixture was concentrated in vacuo and the residue heated with EtOAc to give a white solid. The hot organic solution was decanted and the solid dried in vacuo to give 1-{[(4-chloro-1-guanidino-7-isoquinolinyl)sulphonyl]amino}-D-tert-leucine hydrochloride (109.3 mg, 0.24 mmol) as a white solid.

mp 234-236° C. (dec).

¹H(TFA-d, 400 MHz) δ 1.1 (9H, s), 3.9 (1H, s), 8.35 (1H,d), 8.5 (1H, s), 8.6 (1H, d), 9.1 (1H, s) ppm.

LRMS 414, 416 (MH⁺).

Anal. Found: C, 41.70; H, 4.86; N, 15.01. Calc for C₁₆H₂₀ClN₅O₄S.1.0HCl0.5H₂O: C, 41.84; H, 4.83; N, 15.25.

### Example 28

### (a) N-[(4-chloro-1-guanidino-7-isoquinolinyl)sulphonyl]-L-phenylalanine t-butyl ester

### (b) N-[(4-Chloro-1-guanidino-7-isoquinolinyl)sulphonyl]-L-phenylalanine trifluoroacetate

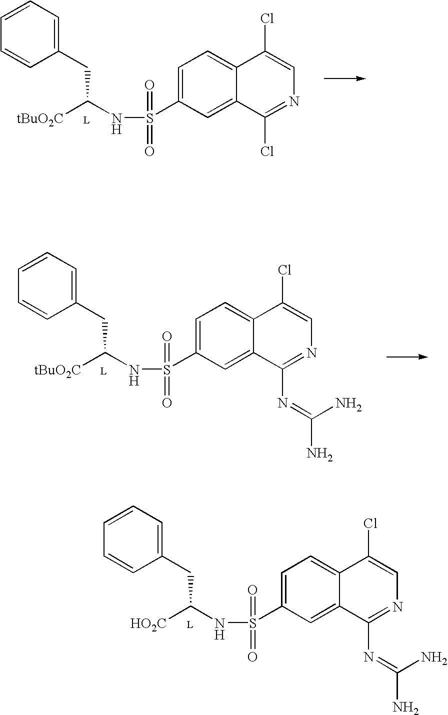

NaH (22 mg, 80% dispersion by wt in mineral oil, 0.73 mmol) was added in one portion to a stirred suspension of guanidine hydrochloride (76.7 mg, 0.80 mmol) in DMSO (5.0 mL) and the mixture was heated at 60° C. under N₂ for 20 min. N-[(1,4-Dichloro-7-isoquinolinyl)sulphonyl]-L-phenylalanine t-butyl ester (103 mg, 0.21 mmol) was added and the mixture heated at 95° C. for 17 h. The solvents were evaporated in vacuo and the residue was purified by column chromatography upon silica gel using CH₂Cl₂-MeOH-0.880NH₃ (95:5:0.5 to 80:20:2) as eluant to give N-[(4-chloro-1-guanidino-7-isoquinolinyl)sulphonyl]-L-phenylalanine t-butyl ester (34.7 mg, 0.069 mmol) as a yellow resin.

¹H (DMSO-d₆, 300 MHz) δ 1.0 (9H, s), 2.7 (1H, dd), 2.8 (1H, dd), 3.9 (1H, dd), 7.1-7.2 (5H, m), 7.1-7.3 (4H, br s), 7.9 (1H, d), 7.95 (1H, d), 8.1 (s, 1H), 8.5 (1H, br d), 8.95 (1H, s) ppm.

LRMS 504, 506 (MH⁺).

N-[(4-Chloro-1-guanidino-7-isoquinolinyl)sulphonyl]-L-phenylalanine t-butyl ester (30 mg, 0.060 mmol) was dissolved in CF₃CO₂H (2.5 mL) and the mixture stirred at room temperature for 2.5 h. The mixture was diluted with CH₂Cl₂ and PhMe, concentrated in vacuo, azeotroping with PhMe, and the residue triturated with Et₂O to give N-[(4-chloro-1-guanidino-7-isoquinolinyl)sulphonyl]-L-phenylalanine trifluoroacetate (24.4 mg, 0.42 mmol) as a white solid.

mp 306° C. (dec).

¹H (DMSO-d₆, 300 MHz) δ 2.7 (1H, dd), 3.0 (1H, dd), 3.95 (1H, m), 6.9-7.1 (5H, m), 7.8-8.4 (4H, br), 7.9 (1H, d), 8.05 (1H, d), 8.3 (s, 1H), 8.6 (1H, br s), 8.8 (1H, s) ppm.

LRMS 448 (MH⁺).

Anal. Found: C, 44.35; H, 3.78; N, 11.38. Calc for C₁₉H₁₈ClN₅O₄S.1.0CF₃CO₂H.0.5H₂O.0.12Et₂O: C, 44.50; H, 3.69; N, 12.08.

### Example 29

### (a) 1-{[(4-chloro-1-guanidino-7-isoquinolinyl)sulphonyl]amino}-O-methyl-D-serine t-butyl ester hydrochloride

### (b) N-[(4-Chloro-1-guanidino-7-isoquinolinyl)sulphonyl]-O-methyl-D-serine hydrochloride

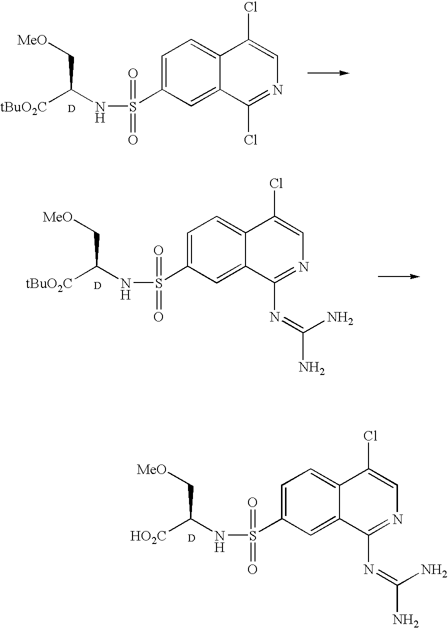

NaH (50 mg, 80% dispersion by wt in mineral oil, 1.66 mmol) was added in one portion to a stirred solution of guanidine hydrochloride (260 mg, 2.72 mmol) in DMSO (4 mL) under N₂ and the mixture was heated at 50° C. for 30 min. 1-{[(1,4-Dichloro-7-isoquinolinyl)sulphonyl]amino}-O-methyl-D-serine t-butyl ester (300 mg, 0.689 mmol) was added in one portion and the mixture heated at 90° C. for 8 h. The cooled mixture was poured into water (50 mL), the aqueous solution was extracted with EtOAc (×2) and the combined organic extracts were washed with water, brine, dried (MgSO₄). The solvents were evaporated in vacuo and the residue purified by column chromatography upon silica gel using CH₂Cl₂-MeOH-0.880NH₃ (90:10:1) as eluant to give the desired product. This material was treated with a solution of HCl in Et₂O (1.0 mL, 1 M), the solvents evaporated in vacuo, and the residue triturated with Et₂O (×2) to give 1-{[(4-chloro-1-guanidino-7-isoquinolinyl)sulphonyl]amino}-O-methyl-D-serine t-butyl ester hydrochloride (18 mg, 0.036 mmol) as a white solid.

¹H (d4-MeOH, 300 MHz) δ 1.2 (9H,s), 3.2 (3H,s), 3.5-3.6 (1H,m), 3.6-3.7 (1H,m), 4.1-4.2 (1H,m), 8.35-8.5 (3H,m), 8.9 (1H,s) ppm.

LRMS 458 (MH⁺).

1-{[(4-Chloro-1-guanidino-7-isoquinolinyl)sulphonyl]amino}-O-methyl-D-serine t-butyl ester hydrochloride (18 mg, 0.036 mmol) was dissolved in a solution of EtOAc saturated with HCl (5 mL) and the mixture stirred at room temperature for 3 h. The mixture was concentrated in vacuo and the residue triturated with EtOAc (×3) to give 1-{[(4-chloro-1-guanidino-7-isoquinolinyl)sulphonyl]amino}-L-tert-leucine hydrochloride (9 mg, 0.02 mmol) as an off-white solid.

¹H (d-TFA, 400 MHz) 3.6 (3H,s), 4.0-4.2 (2H,m), 4.65 (1H, br s), 8.4 (1H,d), 8.5 (1H,s), 8.65 (1H,d), 9.1 (1H,s) ppm.

LRMS 402 (MH⁺).

### Example 30

### (a) N-[(4-chloro-1-guanidino-7-isoquinolinyl)sulphonyl]-D-aspartic acid di-t-butyl ester

### (b) N-[(4-Chloro-1-guanidino-7-isoquinolinyl)sulphonyl]-D-aspartic acid hydrochloride

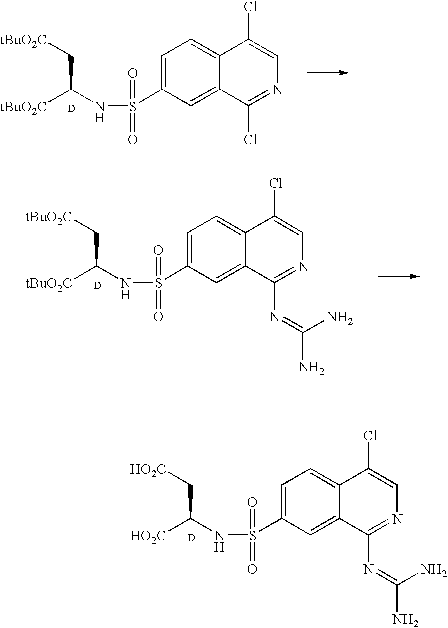

Guanidine hydrochloride (190 mg, 2.0 mmol) was added in one portion to a stirred suspension of NaH (47 mg, 80% dispersion by wt in mineral oil, 1.57 mmol) in DME (7 mL) and the mixture was heated at 60° C. under N₂ for 30 min. 1-{[(1,4-Dichloro-7-isoquinolinyl)sulphonylamino}-D-aspartic acid di-t-butyl ester (250 mg, 0.50 mmol) was added and the mixture heated at reflux for 18 h. The cooled mixture was diluted with EtOAc, washed with water, brine, dried (MgSO₄) and the solvents evaporated in vacuo. The residue was purified by column chromatography upon silica gel using CH₂Cl₂-MeOH-0.880NH₃ (97:3:0.3) as eluant to give N-[(4-chloro-1-guanidino-7-isoquinolinyl)sulphonyl]-D-aspartic acid di-t-butyl ester (50 mg, 0.095 mmol) as a yellow solid.

¹H (CDCl₃, 400 MHz) δ 1.2 (9H, s), 1.4 (9H, s), 2.7 (1H, dd), 2.8 (1H, dd), 4.1 (1H, br t), 8.05 (1H, d), 8.1 (1H, d), 8.15 (1H, s), 9.3 (1H, s) ppm.

LRMS 528, 530 (MH⁺).

N-[(4-chloro-1-guanidino-7-isoquinolinyl)sulphonyl]-D-aspartic acid di-t-butyl ester (50 mg, 0.095 mmol) was dissolved in a solution of EtOAc saturated with HCl (10 mL) and the mixture stirred at room temperature for 4 h. The mixture was concentrated in vacuo and the residue triturated with PhMe and then Et₂O to give N-[(4-chloro-1-guanidino-7-isoquinolinyl)sulphonyl]-D-aspartic acid hydrochloride (29 mg, 0.062 mmol) as an off-white solid.

¹H (CD₃OD, 400 MHz) δ 2.7 (1H, dd), 2.8 (1H, dd), 4.4 (1H, br t), 8.35 (1H, d), 8.45 (1H, s), 8.45 (1H, d), 8.9 (1H, s) ppm.

LRMS 415 (M⁺)

Anal. Found: C, 36.05; H, 3.72; N, 13.62. Calc for C₁₄H₁₄ClN₅O₆S.1.0HCl.0.8H₂O: C, 36.03; H, 3.59; N, 15.01.

### Example 31

### (a) N-[(4-chloro-1-guanidino-7-isoquinolinyl)sulphonyl]-L-proline t-butyl ester

### (b) N-[(4-Chloro-1-guanidino-7-isoquinolinyl)sulphonyl]-L-proline hydrochloride

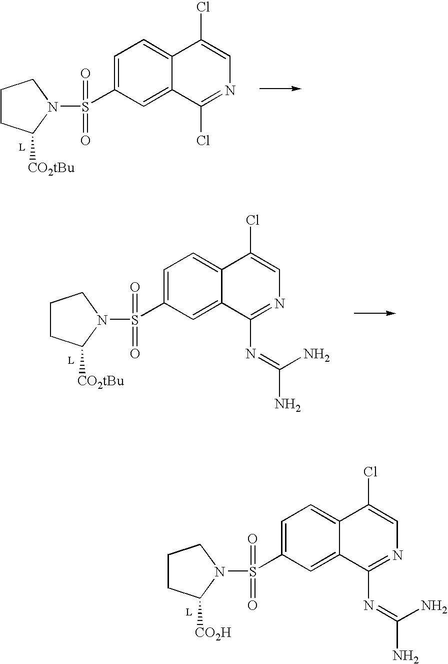

NaH (35 mg, 80% dispersion by wt in mineral oil, 1.16 mmol) was added in one portion to a stirred solution of guanidine hydrochloride (177 mg, 1.85 mmol) in DME (5 mL) and the mixture was heated at 60° C. under N₂ for 45 min. A solution of 1-{[(1,4-dichloro-7-isoquinolinyl)sulphonyl]amino}-L-proline t-butyl ester (200 mg, 0.46 mmol) in DME (2 mL) was added and the mixture heated at 95° C. for 4 h. The solvents were evaporated in vacuo and the residue was purified by column chromatography upon silica gel using pentane-EtOAc (80:20 to 0:100) as eluant, followed by azeotroping with CH₂Cl₂, to give N-[(4-chloro-1-guanidino-7-isoquinolinyl)sulphonyl]-L-proline t-butyl ester (153 mg, 0.32 mmol) as a pale yellow foam.

¹H (DMSO-d₆, 300 MHz) δ 1.35 (9H, s), 1.6-1.7 (1H, m), 1.7-1.9 (2H, m), 1.9-2.0 (1H, m), 3.15-3.25 (1H, m), 3.35-3.5 (1H, m), 4.1 (1H, dd), 7.15-7.4 (4H, br), 8.05 (1H, d), 8.1 (1H, d), 8.1 (1H, s), 9.05 (1H, s) ppm.

LRMS 454, 456 (MH¹), 907 (M₂H⁺).

Anal. Found: C, 50.02; H, 5.41; N, 14.84. Calc for C₁₉H₂₄ClN₅O₄S.0.1EtOAc.0.05CH₂Cl₂: C, 50.02; H, 5.37; N, 15.00.

N-[(4-Chloro-1-guanidino-7-isoquinolinyl)sulphonyl]-L-proline t-butyl ester (60 mg, 0.13 mmol) was dissolved in a solution of EtOAc saturated with HCl (5.0 mL) and the mixture stirred at room temperature for 1 h. The mixture was concentrated in vacuo, azeotroping with EtOAc, and the residue triturated with CH₂Cl₂ to give N-[(4-chloro-1-guanidino-7-isoquinolinyl)sulphonyl]-L-proline hydrochloride (44 mg, 0.095 mmol) as a white powder.

mp 185-189° C.

¹H (DMSO-d₆, 300 MHz) δ 1.5-1.7 (1H, m), 1.7-2.0 (3H, m), 3.2-3.5 (2H, m), 4.2 (1H, dd), 8.3-8.8 (4H, br), 8.2 (2H, s), 8.5 (1H, s), 8.1 (1H, s), 9.05 (1H, s), 11.2 (1H, br) ppm.

Anal. Found: C, 39.89; H, 4.06; N, 14.93. Calc for C₁₅H₁₆ClN₅O₄S.1.0HCl.1.0H₂O.0.1EtOAc: C, 40.11; H, 4.33; N, 15.19.

### Example 32

### (a) N-[(4-chloro-1-guanidino-7-isoquinolinyl)sulphonyl]-D-proline t-butyl ester

### (b) N-[(4-Chloro-1-guanidino-7-isoquinolinyl)sulphonyl]-D-proline hydrochloride

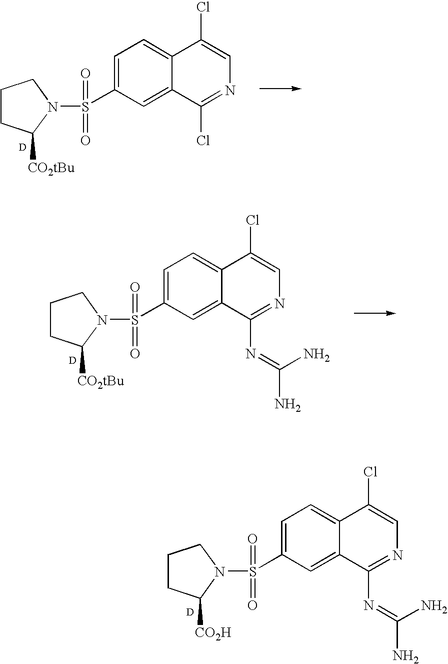

Guanidine hydrochloride (220 mg, 2.3 mmol) was added in one portion to a stirred suspension of NaH (55 mg, 80% dispersion by wt in mineral oil, 1.83 mmol) in DME (8 mL) and the mixture was heated at 60° C. under N₂ for 30 min. 1-{[(1,4-Dichloro-7-isoquinolinyl)sulphonyl]amino}-D-proline t-butyl ester (250 mg, 0.58 mmol) was added and the mixture heated at reflux for 5 h. The cooled mixture was diluted with EtOAc, washed with water, brine, dried (MgSO₄) and the solvents evaporated in vacuo. The residue was purified by column chromatography upon silica gel using CH₂Cl₂-MeOH-0.880NH₃ (97:3:0.3) as eluant to give N-[(4-chloro-1-guanidino-7-isoquinolinyl)sulphonyl]-D-proline t-butyl ester (200 mg, 0.44 mmol) as a yellow solid.

mp>170° C. (dec).

¹H (CDCl₃, 400 MHz) δ 1.45 (9H, s), 1.7-1.8 (1H, m), 1.8-2.05 (3H, m), 3.3-3.6 (1H, m), 4.3 (1H, dd), 6.3-6.6 (4H, br), 8.05 (1H, d), 8.1 (1H, d), 8.1 (1H, s), 9.2 (1H, s) ppm.

LRMS 454, 456 (MH⁺).

Anal. Found: C, 49.57; H, 5.27; N, 14.95. Calc for C₁₉H₂₄ClN₅O₄S.0.2H₂O.0.04CH₂Cl₂: C, 49.61; H, 5.35; N, 15.19.

N-[(4-Chloro-1-guanidino-7-isoquinolinyl)sulphonyl]-D-proline t-butyl ester (50 mg, 0.11 mmol) was dissolved in a solution of EtOAc saturated with HCl (10 mL) and the mixture stirred at room temperature for 2.5 h. The mixture was concentrated in vacuo, azeotroping with CH₂Cl₂, to give N-[(4-chloro-1-guanidino-7-isoquinolinyl)sulphonyl]-D-proline hydrochloride (40 mg, 0.092 mmol) as a white powder.

mp>200° C. (dec). ¹H (CD₃OD, 400 MHz) δ 1.7-1.85 (1H, m), 1.9-2.2 (3H, m), 3.4-3.5 (1H, m), 3.5-3.6 (1H, m), 4.4 (1H, dd), 8.4 (1H, d), 8.45 (1H, s), 8.5 (1H, d), 8.9 (1H, s) ppm.

LRMS 397, 399 (MH⁺)

Anal. Found: C, 40.22; H, 3.92; N, 14.88. Calc for C₁₅H₁₆ClN₅O₄S.1.0HCl.0.2H₂O.0.25CH₂Cl₂: C, 39.89; H, 3.93; N, 15.25.

It was noted that some racemisation had occurred during repetition of the above preparation in some conditions. An alternative route to Example 32(b) was developed, reversing the guanylation/hydrolysis sequence, as exemplified below:
1. Hydrolysis 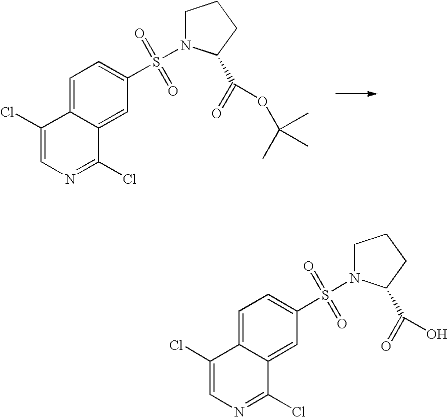
tert-Butyl (2S)-1-[(1,4-dichloro-7-isoquinolinyl)sulfonyl]-2-pyrrolidinecarboxylate (50.0 g, 0.116 mol) was dissolved in conc. HCl (12 M, 200 ml) and stirred for 3.5 h. Water (200 ml) was added over 30 minutes and the resultant white precipitate stirred for a further 0.5 h, filtered and washed with water (3×100 ml). Drying under vacuum gave (2S)-1-[(1,4-dichloro-7-isoquinolinyl)sulfonyl]-2-pyrrolidinecarboxylic acid as a white solid (42.9 g, 0.114 mol).

¹H (d₆-DMSO, 300 MHz) δ 1.6-1.95 (3H, m), 1.95-2.1 (1H, m), 3.25-3.35 (1H, m), 3.35-3.45 (1H, m), 4.3 (1H, dd), 8.35 (2H, s), 8.6 (1H, s), 8.65 (1H, s) ppm.

Chiral analysis was performed using capillary electrophoresis, showing an enantiomeric purity of 97.41%.
2. Guanylation of Free Acid

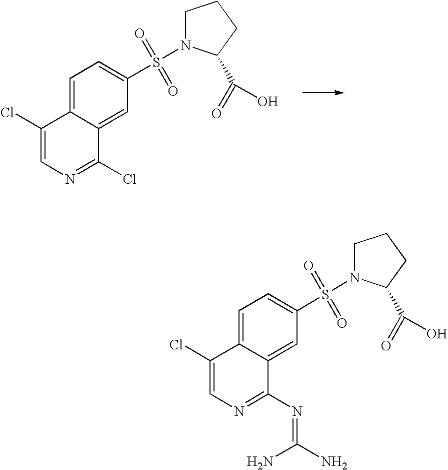

Potassium t-butoxide (49.0 g, 0.0437 mol) and guanidine.HCl (42.8 g, 0.448 mol) in DME (210 ml) was heated to reflux under nitrogen for 20 min. (2S)-1-[(1,4-dichloro-7-isoquinolinyl)sulfonyl]-2-pyrrolidinecarboxylic acid (42.0 g, 0.112 mol) was added and heating continued at reflux for 5.5 h. Water (420 ml) was added and the mixture acidified with c. HCl to pH=5 giving a solid which was removed by filtration, washed with aq. DME (1:1, 2×75 ml) and water (2 x 75 ml) and dried to yield the title compound (b) as a yellow solid (40.71 g, 0.102 mol).

¹H (d₆-DMSO, 300 MHz) δ 1.5-1.65 (1H, m), 1.7-2.0 (3H, m), 3.1-3.25 (1H, m), 3.35-4.05 (1H, m), 4.2 (1H, dd), 7.2-7.7 (4H, br s), 8.0 (1H, d), 8.1-8.2 (2H, m), 9.05 (1H, d).

Chiral analysis was performed using capillary electrophoresis, showing an enantiomeric purity of 99.76% (n=2).

### Example 33

### 4-Chloro-1-guanidino-7-[[(2R)-(hydroxymethyl)-1-pyrrolidinyl]sulphonyl}isoquinoline hydrochloride

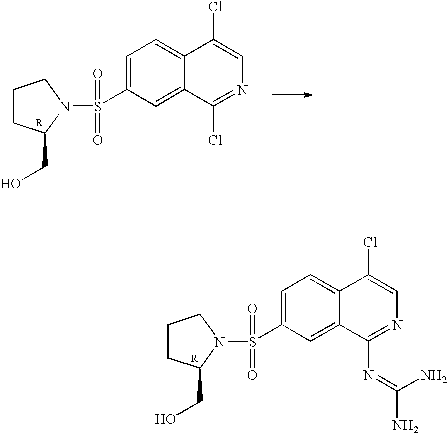

NaH (26 mg, 80% dispersion by wt in mineral oil, 0.87 mmol) was added in one portion to a stirred solution of guanidine hydrochloride (126 mg, 1.32 mmol) in DMSO (2 mL) and the mixture was heated at 50° C. under N₂ for 20 min. A solution of 1,4-dichloro-7-{[(2R)-(hydroxymethyl)-1-pyrrolidinyl]sulphonyl}isoquinoline (120 mg, 0.33 mmol) in DMSO (3 mL) was added in one portion and the mixture heated at 80-90° C. for 1 h. The cooled mixture was poured into water, extracted with EtOAc (2×) and the combined organic extracts were then washed with water (×3), brine, dried (MgSO₄) and evaporated in vacuo. The residue was purified by column chromatography upon silica gel using CH₂Cl₂-MeOH-0.880NH₃ (95:5:0.5 to 80:20:5) as eluant to give the desired product as an off-white, sticky solid. This material was dissolved in MeOH, a solution of HCl in Et₂O (1 M) was added and the solvents were evaporated in vacuo. The residue was recrystallised from MeOH to give 4-chloro-1-guanidino-7-{[(2R)-(hydroxymethyl)-1-pyrrolidinyl]sulphonyl}isoquinoline hydrochloride (43 mg, 0.10 mmol) as a white solid.

mp 275-276.5° C.

¹H (CD₃OD, 400 MHz) δ 1.5-1.65 (2H, m), 1.8-1.95 (2H, m), 3.25-3.35 (2H, m), 3.45-3.55 (1H, m), 3.6-3.65 (1H, m), 3.7-3.85 (2H, m), 8.4 (1H, d), 8.45 (1H, s), 8.5 (1H, d), 8.9 (1H, s) ppm.

LRMS 383 (MH⁺), 405 (MNa⁺), 767 (M₂H⁺).

Anal. Found: C, 42.36; H, 4.54; N, 16.14. Calc for C₁₅H₁₈ClN₅O₃S.1.0HCl.0.25H₂O: C, 42.41; H, 4.63; N, 16.49.

### Example 34

### (a) 1-{[(4-chloro-1-guanidino-7-isoquinolinyl)sulphonyl]amino}isobutyric acid methyl ester

### (b) 2-{[(4-Chloro-1-guanidino-7-isoquinolinyl)sulphonyl]amino}isobutyric acid hydrochloride

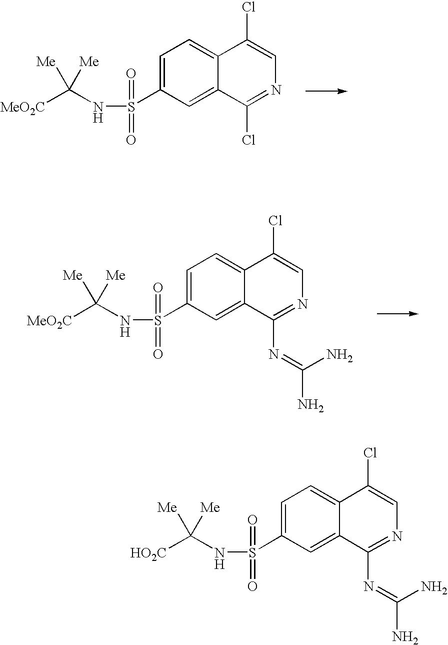

NaH (32 mg, 80% dispersion by wt in mineral oil, 1.07 mmol) was added in one portion to a stirred solution of guanidine hydrochloride (167 mg, 1.7 mmol) in DMSO (5 mL) and the mixture was heated at 50° C. under N₂ for 20 min. 1-{[(1,4-Dichloro-7-isoquinolinyl)sulphonyl]amino}isobutyric acid methyl ester (161 mg, 0.43 mmol) was added in one portion and the mixture heated at 80° C. for 6.5 h. The cooled mixture was poured into water (50 mL), extracted with EtOAc (2×100, 2×25 mL) and the combined organic extracts were washed with water, brine, dried (Na₂SO₄) and evaporated in vacuo. The residue was purified by repeated column chromatography upon silica gel using (i) CH₂Cl₂-MeOH-0.880NH₃ (95:5:0.5), (ii) hexane-EtOAc (70:30), and then (iii) CH₂Cl₂-MeOH-0.880NH₃ (90:10:01), as eluant to give the product as a yellow oil. Trituration with Et₂O gave 1-{[(4-chloro-1-guanidino-7-isoquinolinyl)sulphonyl]amino}isobutyric acid methyl ester (23 mg, 0.054 mmol) as yellow solid.

mp>170° C. (dec).

¹H (CD₃OD, 300 MHz) δ 1.4 (6H, s), 3.5 (3H, s), 8.15-8.25 (3H, m), 9.1 (1H, s) ppm

LRMS 400, 402 (MH⁺).

Anal. Found: C, 44.02; H, 4.65; N, 16.29. Calc for C₁₅H₁₈ClN₅O₄S.0.9H₂O.0.1i-Pr₂O: C, 43.95; H, 5.01; N, 16.43.

A solution of NaOH (1 mL, 2 M, 2 mmol) was added to a solution of 1-{[(4-chloro-1-guanidino-7-isoquinolinyl)sulphonyl]amino}isobutyric acid methyl ester (16.5 mg, 0.041 mmol) in MeOH (0.5 mL) and the mixture was heated at 40-50° C. for 16 h. The cooled mixture was neutrilised with dilute HCl (0.5 mL, 2 M) to give a precipitate. The solid was collected by filtration, with copious water washing, and then dissolved in conc. HCl. The solvents were evaporated in vacuo azeptroping with PhMe, and then dried under high vacuum to give 1-{[(4-chloro-1-guanidino-7-isoquinolinyl)sulphonyl]-amino}isobutyric acid hydrochloride (12 mg, 0.026 mmol) as a pale cream solid.

mp 258° C. (dec)

¹H (CD₃OD, 400 MHz) δ 1.45 (6H, s), 8.4 (1H, d), 8.4 (1H, s), 8.45 (1H, d), 8.9 (1H, s) ppm.

LRMS 386, 388 (MH⁺).

Anal. Found: C, 37.89; H, 4.33; N, 15.18. Calc for C₁₄H₁₆ClN₅O₄S.1.0HCl.1.5H₂O.0.05Et₂O: C, 37.65; H, 4.56; N, 15.46.

### Example 35

### 2-{[(4-Chloro-1-guanidino-7-isoquinolinyl)sulphonyl]amino}-2-methylpropanamide hydrochloride

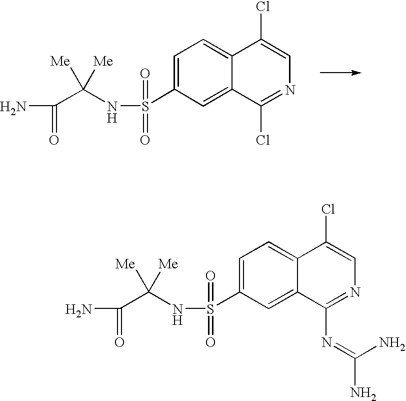

NaH (41 mg, 80% dispersion by wt in mineral oil, 1.36 mmol) was added in one portion to a stirred solution of guanidine hydrochloride (210 mg, 2.2 mmol) in DMSO (10 mL) under N₂ and the mixture was heated at 23° C. for 30 min. 2-{[(1,4-Dichloro-7-isoquinolinyl)sulphonyl]amino}-2-methylpropanamide (225 mg, 0.50 mmol) was added in one portion and the mixture heated at 90° C. for 8 h. The cooled mixture was partially concentrated in vacuo and the residue poured into water. The aqueous solution was extracted with EtOAc (×4) and the combined organic extracts were washed with water, brine, dried (MgSO₄). The solvents were evaporated in vacuo and the residue purified by column chromatography upon silica gel using CH₂Cl₂-MeOH-0.880NH₃ (90:10:1) as eluant to give the desired product. This material was dissolved in MeOH and treated with a solution of HCl in Et₂O (1.0 mL, 1 M) to furnish 2-{[(4-chloro-1-guanidino-7-isoquinolinyl)sulphonyl]amino)-2-methylpropanamide hydrochloride (86 mg, 0.188 mmol) as an off-white powder.

mp 279-281° C.

¹H (TFA-d, 400 MHz) δ 1.6 (6H, s), 8.35 (1H, br s), 8.4 (1H, s), 8.55 (1H, s), 9.1 (1H, br s) ppm.

LRMS 385, 387 (MH⁺).

Anal. Found: C, 39.68; H, 4.81; N, 18.18. Calc for C₁₄H₁₇ClN₆O₃S.1.0HCl.1.2 MeOH: C, 39.71; H, 5.00; N, 18.28.

### Example 36

### (a) Ethyl 1-{[(4-chloro-1-guanidino-7-isoquinolinyl)sulphonyl]amino}cyclobutanecarboxylate

### (b) 1-{[(4-Chloro-1-guanidino-7-isoquinolinyl)sulphonyl]amino}cyclobutanecarboxylic acid hydrochloride

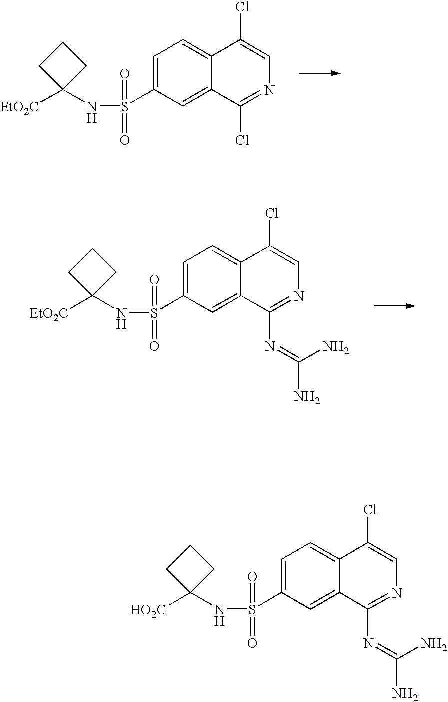

NaH (37 mg, 80% dispersion by wt in mineral oil, 1.24 mmol) was added in one portion to a stirred solution of guanidine hydrochloride (189 mg, 1.98 mmol) in DMSO (6 mL) and the mixture was heated at 60° C. under N₂ for 30 min. Ethyl 1-{[(1,4-dichloro-7-isoquinolinyl)sulphonyl]amino}-cyclobutanecarboxylate (200 mg, 0.50 mmol) was added in one portion and the mixture heated at 80° C. for 10 h. The cooled mixture was poured into water, extracted with EtOAc (2×50 mL) and the combined organic extracts were dried (MgSO₄) and evaporated in vacuo. The residue was purified by column chromatography upon silica gel using pentane-EtOAc (50:50 to 0:100) as eluant to give ethyl

1-{[(4-chloro-1guanidino-7-isoquinolinyl)sulphonyl]amino}cyclobutanecarboxylate (150 mg, 0.34 mmol) as a yellow powder.

mp 165-169° C.

¹H (DMSO-d₆, 300 MHz) δ 1.0 (3H, t), 1.6-1.8 (2H, m), 2.05-2.2 (2H, m), 2.25-2.4 (2H, m), 3.8 (2H, q), 7.0-7.4 (4H, br), 8.05 (2H, s), 8.1 (1H, s), 8.6 (1H, s), 9.05 (1H, s) ppm.

LRMS 426, 428 (MH⁺).

Anal. Found: C, 46.62; H, 4.62; N, 15.82. Calc for C₁₇H₂₀ClN₅O₄S.0.25CH₂Cl₂: C, 46.65; H, 4.63; N, 15.70.

A solution of NaOH (5 mL, 2 M, 10 mmol) was added to a solution of ethyl 1-{[(4-chloro-1-guanidino-7-isoquinolinyl)sulphonyl]amino}cyclobutanecarboxylate (100 mg, 0.23 mmol) in MeOH (5 mL) and the mixture was heated at 55° C. for 6 h. The cooled mixture was neutrilised with dilute HCl (5 mL, 2 M) to give a precipitate and the MeOH was evaporated in vacuo. The solid was collected by filtration, with copious water washing, and dried under high vacuum to give 1-{[(4-chloro-1-guanidino-7-isoquinolinyl)sulphonyl]amino}cyclobutanecarboxylic acid hydrochloride (15 mg, 0.033 mmol).

¹H (DMSO-d₆, 400 MHz) δ 1.65-1.8 (2H, m), 2.05-2.2 (2H, m), 2.25-2.4 (2H, m), 8.3 (1H, d), 8.35-8.7 (4H, br), 8.4 (1H, d), 8.5 (1H, s), 8.7 (1H, s), 8.95 (1H, s), 11.0 (1H, br), 12.5 (1H, br) ppm.

Anal. Found: C, 40.06; H, 4.34; N, 15.09. Calc for C₁₅H₁₆ClN₅O₄S.1.0HCl.1.0H₂O: C, 39.83; H, 4.23; N, 15.48.

### Example 37

### (a) N-[(4-chloro-1-guanidino-7-isoquinolinyl)sulphonyl]cyclo-leucine ethyl ester

### (b) N-[(4-Chloro-1-guanidino-7-isoquinolinyl)sulphonyl]cycloleucine

### (c) N-[(4-chloro-1-guanidino-7-isoquinolinyl)sulphonyl]cycloleucine trifluoroacetate

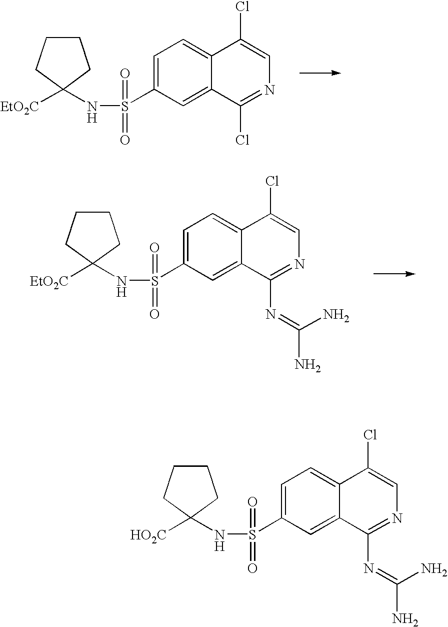

NaH (1.12 g, 80% dispersion by wt in mineral oil, 37.3 mmol) was added portionwise to a stirred suspension of guanidine hydrochloride (5.85 g, 59.4 mmol) in DMSO (320 mL) and the mixture was heated at 30-50° C. under N₂ for 30 min. N-[(1,4-Dichloro-1-guanidino-7-isoquinolinyl)sulphonyl]-cycloleucine ethyl ester (6.2 g, 14.9 mmol) was added in one portion and the mixture heated at 80° C. for 8 h. The cooled mixture concentrated in vacuo to ca. 160 mL and poured into water (800 mL). The aqueous mixture was extracted with EtOAc (4×150 mL) and the combined organic extracts were then washed with water, brine, dried (MgSO₄) and evaporated in vacuo. The residue was purified by column chromatography upon silica gel using CH₂Cl₂-MeOH-0.880NH₃ (95:5:0.5 to 90:10:1) as eluant and then recrystallised from EtOAc to give N-[(4-chloro-1-guanidino-7-isoquinolinyl)sulphonyl]cyclo-leucine ethyl ester (1.43 g, 3.25 mmol) as a yellow solid.

mp 225-226° C.

¹H (DMSO-d₆, 300 MHz) δ 1.1 (3H, t), 1.35-1.45 (2H, m), 1.45-1.5 (2H, m), 1.85-1.95 (4H, br), 3.9 (2H, q), 7.1-7.35 (4H, br), 8.0 (1H, d), 8.05 (1H, d), 8.1 (1H, s), 9.1 (1H, s) ppm.

LRMS 440, 442 (MH⁺).

Anal. Found: C, 49.02; H, 4.97; N, 15.61. Calc for C₁₈H₂₂ClN₅O₄S: C, 49.14; H, 5.04; N, 15.92.

A solution of NaOH (75 mL, 2 M, 150 mmol) was added to a solution of N-[(4-chloro-1-guanidino-7-isoquinolinyl)sulphonyl]cycloleucine ethyl ester (1.39 g, 3.16 mmol) in MeOH (75 mL) and the mixture heated at 40-50° C. for 24 h. The cooled mixture was neutrilised with dilute HCl (75 mL, 2 M) to give a precipitate and the MeOH was evaporated in vacuo. The solid was collected by filtration, with copious water washing, and dried under high vacuum to give N-[(4-chloro-1-guanidino-7-isoquinolinyl)sulphonyl]cycloleucine (1.27 g, 3.08 mmol) as a white powder.

Anal. Found: C, 46.40; H, 4.39; N, 16.66. Calc for C₁₆H₁₈ClN₅O₄S: C, 46.66; H, 4.41; N, 17.00.

N-[(4-Chloro-1-guanidino-7-isoquinolinyl)sulphonyl]cycloleucine (8 mg) was dissolved in CF₃CO₂H (ca. 1.0 mL) and the mixture was evaporated in vacuo, azeotroping with PhMe. The residue was triturated with i-Pr₂O and Et₂O to give a white solid. The solid was dissolved in MeOH, filtered and the filtrate evaporated in vacuo to give N-[(4-chloro-1-guanidino-7-isoquinolinyl)sulphonyl]cycloleucine trifluoroacetate (12 mg).

mp>178° C. (dec).

¹H (DMSO-d₆, 400 MHz) δ 1.3-1.45 (2H, m), 1.45-1.55 (2H, m), 1.85-1.95 (4H, br), 8.25-8.6 (4H, br), 8.3 (1H, d), 8.4 (1H, d), 8.5 (1H, s), 8.85 (1H, s), 10.8 (1H, br), 12.4 (1H, br) ppm.

LRMS 412, 414 (MH⁺).

Anal. Found: C, 39.50; H, 3.62; N, 11.50. Calc for C₁₆H₁₈ClN₅O₄S.1.0CF₃CO₂H.1.0H₂O: C, 39.75; H, 3.89; N, 12.88.

### Example 38

### 1-{[(4-Chloro-1-guanidino-7-isoquinolinyl)sulphonyl]amino}-N-(2-hydroxyethyl)cyclopentanecarboxamine hydrochloride

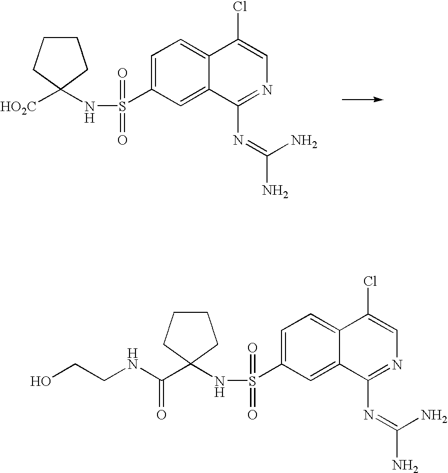

(COCl)₂ (60 μL, 0.67 mmol) and then DMF (3 drops) were added to a stirred suspension of N-[(4-chloro-1-guanidino-7-isoquinolinyl)sulphonyl]cycloleucine hydrochloride (150 mg, 0.334 mmol) in CH₂Cl₂ (15 mL) and the mixture was stirred at 23° C. for 30 min. The solvents were evaporated in vacuo, azeotroping with PhMe, to give the corresponding acid chloride.This material was redissolved in CH₂Cl₂ (15 mL) and added to a stirred solution of 2-hydroxyethylamine (400 μL) in CH₂Cl₂ (15 mL) and the mixture stirred for 1 h. The solvents were evaporated in vacuo and the residue was purified by column chromatography upon silica gel using CH₂Cl₂-MeOH-0.880NH₃ (90:10:1) as eluant to give 1-{[(4-chloro-1-guanidino-7-isoquinolinyl)sulphonyl)amino}-N-(2-hydroxyethyl)cyclopentanecarboxamine. This material was dissolved in EtOAc-EtOH and a solution of HCl in Et₂O (1 M) was added which gave a precipitate. The solvents were decanted and the solid was triturated with Et₂O, collected by filtration and dried to give 1-{((4-chloro-1-guanidino-7-isoquinolinyl)sulphonyl]amino})-N-(2-hydroxyethyl)cyclopentanecarboxamine hydrochloride (77 mg, 0.155 mmol) as a white solid.

mp 244-246° C.

¹H (CD₃OD, 300 MHz) δ 1.35-1.5 (2H, m), 1.5-1.65.(2H, m), 1.85-2.0 (2H, m), 2.0-2.15 (2H, m), 3.1-3.2 (2H, m), 3.5-3.65 (2H, m), 8.4 (1H, d), 8.45 (1H, s), 8.5 (1H, d), 8.95 (1H, s) ppm.

LRMS 455 (MH⁺), 477 (MNa⁺).

Anal. Found: C, 43.63; H, 5.03; N, 16.65. Calc for C₁₈H₂₃ClN₆O₄S.1.0HCl.0.25 H₂O: C, 43.60; H, 4.98; N, 16.95.

### Example 39

### (a) 1-{[(4-Chloro-1-guanidino-7-isoquinolinyl)sulphonyl]amino}-N-[2-(dimethylamino)ethyl]cyclopentanecarboxamine

### (b) 1-{[(4-Chloro-1-guanidino-7-isoquinolinyl)sulphonyl]amino}-N-[2-(dimethylamino)ethyl]cyclopentanecarboxamine dihydrochloride

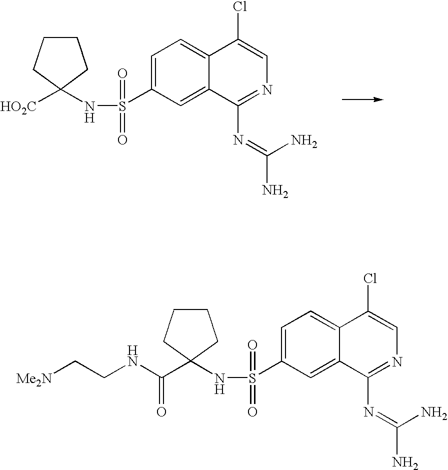

A solution HCl in Et₂O (0.5 mL, 1 M) was added to a stirred solution of N-[(4-chloro-1-guanidino-7-isoquinolinyl)sulphonyl]cycloleucine (100 mg, 0.243 mmol) in MeOH. The solvents were evaporated in vacuo and the residue azeotroped with PhMe to give the corresponding hydrochloride salt.

(COCl)₂ (42 μL, 0.48 mmol) and then DMF (2 drops) were added to a stirred solution of N-[(4-chloro-1-guanidino-7-isoquinolinyl)sulphonyl]cycloleucine hydrochloride (0.243 mmol) in CH₂Cl₂ (5 mL) and the mixture was stirred at 23° C. for 18 h. The solvents were evaporated in vacuo, the residue redissolved in CH₂Cl₂ (5 mL), and 2-(dimethylamino)ethylamine (60 μL, 0.48 mmol) was added and the mixture stirred for 3 h. The solvents were evaporated in vacuo and the residue partioned between EtOAc and aqueous NaHCO₃ (10%). The organic phase was dried and evaporated. The residue was purified by column chromatography upon silica gel using CH₂Cl₂-MeOH-0.880NH₃ (95:5:0.5 to 90:10:1) as eluant to give 1-{[(4-chloro-1-guanidino-7-isoquinolinyl)sulphonyl]amino}-N-[2-(dimethylamino)ethyl]cyclopentanecarboxamine.

LRMS 482 (MH⁺).

This material was dissolved in EtOAc, a solution of HCl in Et₂O (1 M) was added and the solvents were evaporated in vacuo to give 1-{[(4-chloro-1-guanidino-7-isoquinolinyl)sulphonyl]amino}-N-[2-(dimethylamino)ethyl]cyclopentanecarboxamine dihydrochloride (28 mg, 0.048 mmol) as a white solid.

¹H (TFA-d, 400 MHz) δ 1.5 (2H, br s), 1.7 (2H, br s), 2.1 (4H, br s), 3.2 (6H, s), 3.7 (2H, br s), 4.0 (2H, br s), 7.8 (1H, br s), 8.45 (1H, d), 8.5 (1H, s), 8.6 (1H, d), 9.5 (1H, s) ppm.

LRMS 482 (MH⁺).

Anal. Found: C, 41.25; H, 5.63; N, 16.59. Calc for C₂₀H₂₈ClN₇O₃S.2.0HCl.1.5H₂O: C, 41.28; H, 5.72; N, 16.85.

### Example 40

### 4-Chloro-1-guanidino-N-[1-(hydroxymethyl)cyclopentyl]-7-isoquinolinesulphonamide hydrochloride

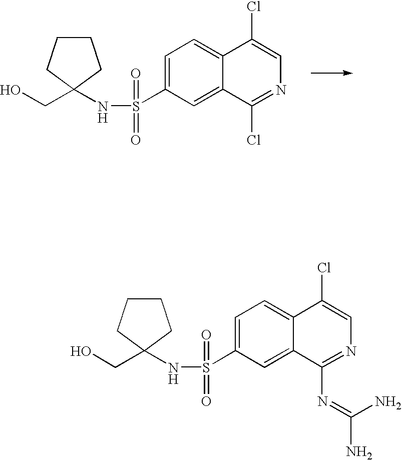

NaH (30 mg, 80% dispersion by wt in mineral oil, 1.0 mmol) was added in one portion to a stirred solution of guanidine hydrochloride (157 mg, 1.6 mmol) in DMSO (5 mL) and the mixture was heated at 60° C. under N₂ for 20 min. 1,4-Dichloro-N-[1-(hydroxymethyl)cyclopentyl]-7-isoquinolinesulphonamide (150 mg, 0.40 mmol) was added in one portion and the mixture heated at 80° C. for 4 h. A second portion of guanidine (0.40 mmol)[prepared from guanidine hydrochloride (38 mg) and NaH (12 mg)] in DMSO (1 mL) was added and the mixture heated at 80° C. for an additional 6 h. The cooled mixture was poured into water (80 mL), extracted with EtOAc (2×50 mL) and the combined organic extracts were then washed with brine, dried (MgSO₄) and evaporated in vacuo. The residue was purified by column chromatography upon silica gel using CH₂Cl₂-MeOH-0.880NH₃ (97.5:2.5:0.25 to 80:20:5) as eluant to give the partially purified product (90 mg). This material was converted to the corresponding hydrochloride salt by treatment with a solution of HCl in Et₂O (1 M) and then recrystallised from EtOH to give 4-chloro-1-guanidino-N-[1-(hydroxymethyl)cyclopentyl]-7-isoquinolinesulphonamide hydrochloride (16 mg, 0.040 mmol) as a white solid.

mp 245-247° C.

¹H (CD₃OD, 400 MHz) δ 1.4-1.55 (4H, m), 1.55-1.7 (2H, m), 1.8-1.9 (2H, m), 3.5 (2H, s), 8.4 (1H, d), 8.45 (1H, s), 8.45 (1H, d), 8.9 (1H, s) ppm.

LRMS 398, 400 (MH⁺).

Anal. Found: C, 44.17; H, 4.84; N, 15.88. Calc for C₁₆H₂₀ClN₅O₃S.1.0HCl: C, 44.24; H, 4.87; N, 16.12.

### Example 41

### (a) N-[(4-chloro-1-guanidino-7-isoquinolinyl)sulphonyl]-N-[2-(dimethylamino)ethyl]cycloleucine ethyl ester dihydrochloride

### (b) N-[(4-Chloro-1-guanidino-7-isoquinolinyl)sulphonyl]-N-[2-(dimethylamino)ethyl]cycloleucine dihydrochloride

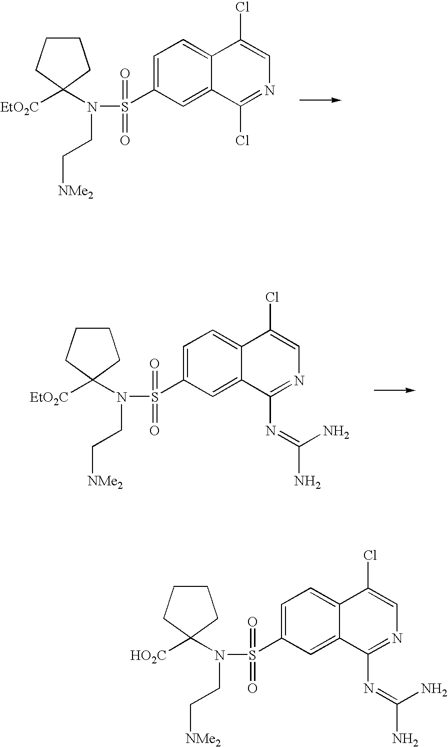

NaH (32 mg, 80% dispersion by wt in mineral oil, 1.05 mmol) was added in one portion to a stirred solution of guanidine hydrochloride (145 mg, 1.52 mmol) in DMSO (4 mL) and the mixture was heated at 50° C. under N₂ for 20 min. N-[(1,4-Dichloro-7-isoquinolinyl)sulphonyl]-N-[2-(dimethylamino)ethyl]cycloleucine ethyl ester hydrochloride (160 mg, 0.305 mmol) was added in one portion and the mixture heated at 90° C. for 1 h. The cooled mixture was poured into water, extracted with EtOAc (2×20 mL) and the combined organic extracts were then washed with brine, dried (Na₂SO₄) and evaporated in vacuo. The residue was dissolved in Et₂O, filtered, and a solution of HCl in Et₂O (1 M) was added which gave a precipitate. The solvents were evaporated in vacuo and the residue recrystallised from hot EtOH to give N-[(4-chloro-1-guanidino-7-isoquinolinyl)sulphonyl]-N-[2-(dimethylamino)ethyl]cycloleucine ethyl ester dihydrochloride (123 mg, 0.20 mmol) as a pale yellow solid.

mp 228-230° C.

¹H (TFA-d, 400 MHz) δ 1.45 (3H, t), 1.7 (2H, br s), 1.9 (2H, br s), 2.2 (2H, br s), 2.5 (2H, br s), 3.3 (6H, s), 3.75 (2H, br s), 4.3 (2H, br s), 4.4 (2H, q), 8.15 (1H, br s), 8.4 (1H, d), 8.5 (1H, s), 8.65 (1H, d), 9.35 (1H, s) ppm.

LRMS 511, 513 (MH⁺).

Anal. Found: C, 43.74; H, 5.88; N, 13.75. Calc for C₂₂H₃₁ClN₆O₄S.2.0HCl.1.0H₂O: C, 43.90; H, 5.86; N, 13.96.

A solution of NaOH (5 mL, 5 M) was added to a solution of N-[(4-chloro-1-guanidino-7-isoquinolinyl)sulphonyl]-N-[2-(dimethylamino)ethyl]cycloleucine ethyl ester dihydrochloride (75 mg, 0.128 mmol) in dioxane (5 mL) and the mixture was heated at 80° C. for 30 h. The cooled mixture was diluted with water (20 mL), the dioxane was evaporated in vacuo, and the aqueous residue neutrilised with dilute HCl (2 M) to pH 6. The precipitate was collected by filtration with water washing, and then dissolved in MeOH, filtered and evaporated in vacuo. The residue was purified by column chromatography upon silica gel using CH₂Cl₂-MeOH-0.880NH₃ (90:10:1 to 80:20:5) as eluant to give to give the desired product. This material was dissolved in MeOH-EtOAc, a solution of HCl in Et₂O (1 M) was added and the solvents were evaporated in vacuo. The residue was triturated with EtOAc to give N-[(4-chloro-1-guanidino-7-isoquinolinyl)sulphonyl]-N-[2-(dimethylamino)ethyl]cycloleucine dihydrochloride (15.4 mg, 0.025 mmol).

¹H (TFA-d, 400 MHz) δ 1.7 (2H, br s), 1.9 (2H, br s), 2.2 (2H, br s), 2.6 (2H, br s), 3.25 (6H, s), 3.8 (2H, br s), 4.3 (2H, br s), 8.1 (1H, br s), 8.4 (1H, d), 8.5 (1H, s), 8.65 (1H,d), 9.4 (1H, s) ppm.

LRMS 483 (MH⁺).

Anal. Found: C, 39.03; H, 5.60; N, 14.02. Calc for C₂₀H₂₇ClN₆O₄S.2HCl.3H₂O: C, 39.38; H, 5.78; N, 13.78.

### Example 42

### N-(t-Butoxycarbonylmethyl)-N-[(4-chloro-1-guanidino-7-isoquinolinyl)sulphonyl]cycloleucine ethyl ester

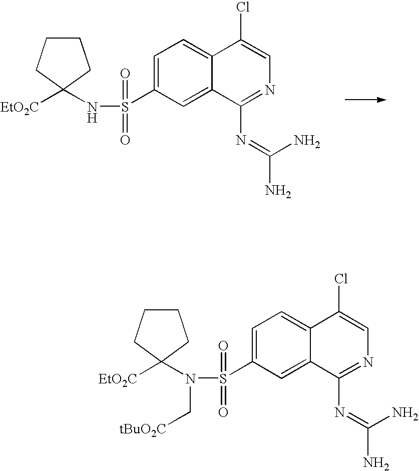

Anhydrous K₂CO₃ (34 mg, 0.25 mmol) and t-butyl bromoacetate (44 μL, 0.30 mmol) were added to a stirred solution of N-[(4-chloro-1guanidino-7-isoquinolinyl)sulphonyl]cycloleucine ethyl ester (110 mg, 0.25 mmol) in DMF (1.0 mL) and the mixture was stirred at 23° C. for 18 h. The mixture was diluted with EtOAc (60 mL), washed with water (3×100 mL), dried (MgSO₄) and evaporated in vacuo. The residue was purified by column chromatography upon silica gel using pentane-EtOAc (100:0 to 20:80) as eluant to give N-(t-butoxycarbonylmethyl)-N-[(4-chloro-1-guanidino-7-isoquinolinyl)sulphonyl]cycloleucine ethyl ester (95 mg, 0.17 mmol) as a white solid.

¹H (CDCl₃, 400 MHz) δ 1.3 (3H, t), 1.45 (9H, s), 1.6-1.7 (4H, m), 1.85-1.95 (2H, br), 2.25-2.35 (2H, m), 4.2 (2H, q), 4.5 (2H, s), 8.1 (1H, d), 8.15 (1H, s), 8.3 (1H, dd), 9.3 (1H, d) ppm.

LRMS 554 (MH⁺).

Anal. Found: C, 52.31; H, 5.94; N, 13.33. Calc for C₂₄H₃₂ClN₅O₆S: C, 52.03; H, 5.82; N, 12.64.

### Example 43

### (a) Methyl 1-{[(4-chloro-1-guanidino-7-isoquinolinyl)sulphonyl]amino}cyclohexanecarboxylate

### (b) 1-{[(4-Chloro-1-guanidino-7-isoquinolinyl)sulphonyl]amino}cyclohexanecarboxylic acid hydrochloride

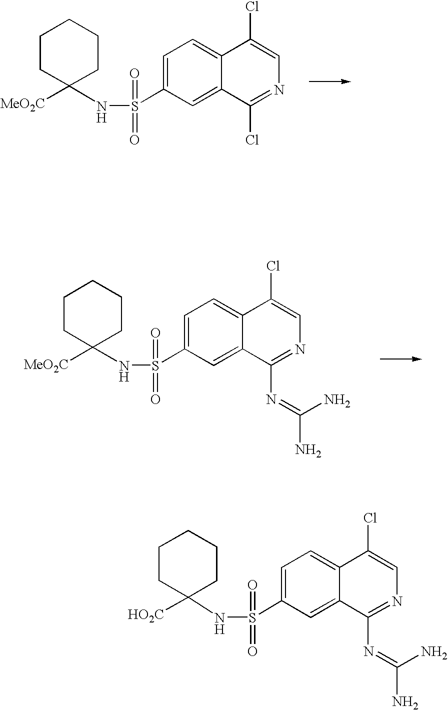

NaH (22.3 mg, 80% dispersion by wt in mineral oil, 0.743 mmol) was added in one portion to a stirred solution of guanidine hydrochloride (117 mg, 1.98 mmol) in DMSO (5 mL) and the mixture was heated at 50-70° C. under N₂ for 25 min. Methyl 1-{[(1,4-dichloro-7-isoquinolinyl)sulphonyl]amino}-cyclohexanecarboxylate (124 mg, 0.30 mmol) was added in one portion and the mixture heated at 80° C. for 8 h. The cooled mixture was poured into water (50 mL), extracted with EtOAc (2×50 mL) and the combined organic extracts were washed with water, brine, dried (MgSO₄) and evaporated in vacuo. The residue was crystallised from a minimum of hot EtOAc to give methyl 1-{[(4-chloro-1-guanidino-7-isoquinolinyl)sulphonyl]amino}cyclohexanecarboxylate (12 mg, 0.043 mmol) as yellow solid. Evaporation of the mother liquors and trituration of the residue with Et₂O gave a second crop (7 mg).

mp>220° C. (dec).

¹H (DMSO-d₆, 400 MHz) δ 1.1-1.35 (6H, m), 1.65-1.75 (2H, m), 1.75-1.85 (2H, m), 3.35 (3H, s), 7.1-7.4 (4H, br), 8.0 (1H, d), 8.05 (1H, d), 8.1 (1H, s), 8.15 (1H, s), 9.0 (1H, s) ppm.

LRMS 440, 442 (MH⁺).

Anal. Found: C, 48.55; H, 5.12; N, 15.73. Calc for C₁₈H₂₂ClN₅O₄S.0.3H₂O: C, 49.14; H, 5.04; N, 15.92.

A solution of NaOH (1 mL, 2 M, 2 mmol) was added to a solution of methyl 1-{[(4-chloro-1-guanidino-7-isoquinolinyl)sulphonyl]amino}cyclohexanecarboxylate (12 mg, 0.027 mmol) in MeOH (4 mL) and the mixture was heated at 50-60° C. for 4 d. The cooled mixture was neutrilised with dilute HCl (1 mL, 2 M) to give a precipitate. The solid was collected by filtration, with copious water washing, and then triturated with EtOAc. The solid was dissolved in conc. HCl, the solvents were evaporated in vacuo azeptroping with PhMe, and then dried under high vacuum to give 1-{[(4-chloro-1-guanidino-7-isoquinolinyl)sulphonyl]amino}cyclohexanecarboxylic acid hydrochloride (11 mg, 0.021 mmol).

mp 194° C. (dec)

¹H (DMSO-d₆, 400 MHz) δ 1.1-1.4 (6H, m), 1.6-1.8 (2H, m), 1.8-1.95 (2H, m), 8.15-8.7 (4H, br), 8.2 (1H, s), 8.3 (1H, d), 8.4 (1H, d), 8.45 (1H, s), 8.9 (1H, s), 10.9 (1H, br), 12.4 (1H, br) ppm.

LRMS 426 (MH⁺).

Anal. Found: C, 39.87; H, 5.05; N, 13.16. Calc for C₁₇H₂₀ClN₅O₄S.1.0HCl.3.0H₂O: C, 39.54; H, 5.27; N, 13.56.

### Example 44

### (a) Methyl 4-{[(4-chloro-1-guanidino-7-isoquinolinyl)sulphonyl]amino}tetrahydro-2H-pyran-4-carboxylate

### (b) 4-{[(4-Chloro-1-guanidino-7-isoquinolinyl)sulphonyl]amino}tetrahydro-2H-pyran-4-carboxylic acid hydrochloride

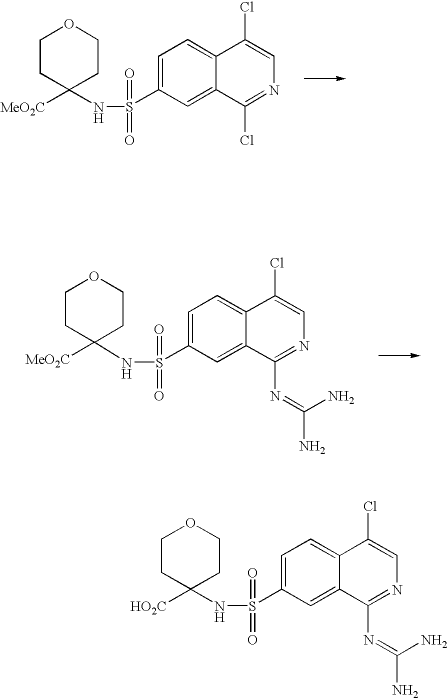

NaH (33.5 mg, 80% dispersion by wt in mineral oil, 1.12 mmol) was added in one portion to a stirred solution of guanidine hydrochloride (176 mg, 1.84 mmol) in DMSO (3.0 mL) under N₂ and the mixture was heated at 50° C. for 15 min. Methyl 4-{[(1,4-dichloro-7-isoquinolinyl)sulphonyl]amino}tetrahydro-2H-pyran-4-carboxylate (187 mg, 0.446 mmol) was added in one portion and the mixture heated at 80° C. for 8 h. A second portion of guanidine (0.45 mmol)[prepared from guanidine hydrochloride and NaH] in DMSO (1.0 mL) was added and the mixture heated at 90° C. for an additional 4 h. The cooled mixture was poured into water (100 mL), extracted with EtOAc (3×50 mL) and the combined organic extracts were washed with brine, dried (Na₂SO₄). The solvents were evaporated in vacuo and the residue purified by column chromatography upon silica gel using CH₂Cl₂-MeOH-0.880NH₃ (95:5:0.5) as eluant, and then crystallised with EtOAc, to give to give methyl 4-{[(4-chloro-1-guanidino-7-isoquinolinyl)sulphonyl]amino}tetrahydro-2H-pyran-4-carboxylate (83 mg, 0.186 mmol) as a yellow solid.

mp 245-247° C.

¹H (CDCl₃, 400 MHz) δ 3.3 (3H, s), 3.35-3.45 (8H, m), 7.1-7.4 (4H, br), 8.05 (2H, s), 8.1 (1H, s), 8.4 (1H, s), 9.0 (1H, s) ppm.

LRMS 442, 444 (MH⁺).

Anal. Found: C, 46.18; H, 4.56; N, 15.32. Calc for C₁₇H₂₀ClN₅O₃S.0.2H₂O: C, 45.83; H, 4.62; N, 15.72.

A solution of NaOH (1 mL, 2 M, 2 mmol) was added to a solution of methyl 4-{[(4-chloro-1-guanidino-7-isoquinolinyl)sulphonyl]amino}tetrahydro-2H-pyran-4-carboxylate (68 mg, 0.153 mmol) in MeOH (12 mL) and the mixture was heated at reflux for 30 h. The cooled mixture was neutrilised with dilute HCl (1 mL, 2 M), partially concentrated by evaporation in vacuo to give a precipitate which was collected by filtration, with water washing. The solid was extracted with warm conc. HCl, the solution decanted from insoluble material and the solvents were evaporated in vacuo. The solid residue was azeptroped with PhMe and then dried under high vacuum to give 4-{[(4-chloro-1-guanidino-7-isoquinolinyl)sulphonyl]amino}tetrahydro-2H-pyran-4-carboxylate acid hydrochloride (30 mg, 0.062 mmol) as a white solid.

mp 190-210° C. (dec).

¹H (DMSO-d₆, 400 MHz) δ 3.2-3.5 (8H, m), 8.2-8.7 (4H, br), 8.3 (1H, d), 8.4 (1H, d), 8.45 (1H, s), 8.95 (1H, s), 11.0 (1H, br s), 12.6 (1H, br s) ppm.

Anal. Found: C, 39.76; H, 4.33; N, 14.12. Calc for C₁₆H₁₈ClN₅O₅S.1.0HCl1.1H₂O: C, 39.69; H, 4.41; N, 14.47.

### Example 45

### (a) t-Butyl (±)-cis-2-{[(4-chloro-1-guanidino-7-isoquinolinyl)sulphonyl]amino}-cyclohexanecarboxylate

### (b) (±)-cis-2-{[(4-Chloro-1-guanidino-7-isoquinolinyl)sulphonyl]amino}cyclohexanecarboxylic acid hydrochloride

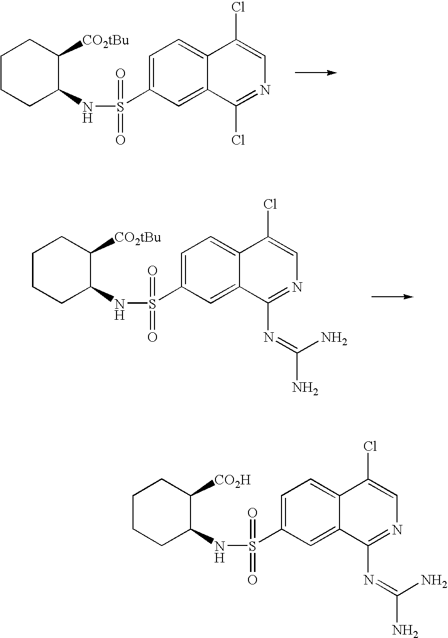

Guanidine hydrochloride (325 mg, 3.4 mmol) was added in one portion to a stirred suspension of NaH (89 mg, 80% dispersion by wt in mineral oil, 2.97 mmol) in DME (5 mL) and the mixture was heated at 60° C. under N₂ for 30 min. A solution of t-butyl (±)-cis-2-{[(1,4-dichloro-7-isoquinolinyl)sulphonyl]amino}cyclohexanecarboxylate (391 mg, 0.85 mmol) in DME (5 mL) was added and the mixture heated at 90° C. for 6 h. The solvents were evaporated in vacuo, the residue was dissolved with EtOAc, washed with aqueous NH₄Cl, dried (MgSO₄) and evaporated in vacuo. The residue was purified by column chromatography upon silica gel using using toluene-i-PrOH-0.880NH₃ (100:0:0 to 90:10:0.05) as eluant to give t-butyl (±)-cis-2-{[(4-chloro-1-guanidino-7-isoquinolinyl)sulphonyl]amino}cyclohexanecarboxylate (75 mg, 0.15 mmol) as a white solid.

¹H (CDCl₃, 400 MHz) δ 1.1-1.8 (7H, mm), 1.4 (9H, s), 1.95 (1H, m), 2.55 (1H, dd), 3.45 (1H, br s), 5.9 (1H, d), 6.0-6.5 (4H, br), 8.05 (1H, d), 8.1 (1H, d), 8.15 (1H, s), 9.3 (1H, s) ppm.

LRMS 482, 484 (MH⁺).

CF₃CO₂H (3.0 mL) was added to a stirred solution of t-butyl (±)-cis-2-{[(4-chloro-1-guanidino-7-isoquinolinyl)sulphonyl]amino}cyclohexanecarboxylate (66 mg, 0.14 mmol) in CH₂Cl₂ (3.0 mL) and the mixture was stirred at 23° C. for 6 h. The solvents were evaporated in vacuo, azeotroping CH₂Cl₂ (×3). The residue was dissolved in EtOAc and a solution of HCl in Et₂O (200 μL, 1.0 M) was added which gave a precipitate. The white solid was collected by filtration and dried to give (±)-cis-2-{[(4-chloro-1-guanidino-7-isoquinolinyl)sulphonyl]amino}cyclohexanecarboxylic acid hydrochloride (35 mg, 0.069 mmol).

mp 220-223° C. (dec).

¹H (DMSO-d₆, 400 MHz) δ 1.1-1.3 (3H, m), 1.4-1.6 (4H, m), 1.7-1.8 (1H, m), 2.5 (1H, m), 3.75 (1H, br s), 8.0 (1H, d), 8.25-8.6 (4H, br), 8.35 (2H, s), 8.45 (1H, s), 8.95 (1H, s) ppm.

Anal. Found: C, 42.95; H, 4.96; N, 13.79. Calc for C₁₇H₂₀ClN₅O₄S.1.0HCl.1.25H₂O.0.3Et₂O: C, 43.11; H, 5.27; N, 13.81.

### Example 46

### Ethyl (±)-trans-2-{[(4-chloro-1-guanidino-7-isoquinolinyl)sulphonyl]amino}cyclohexanecarboxylate

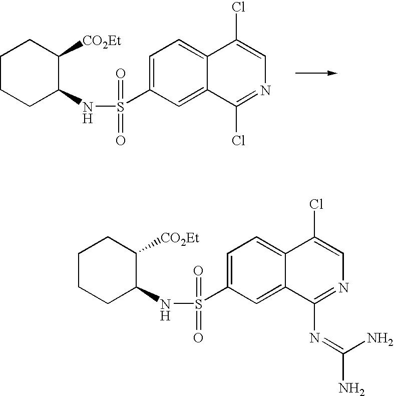

Guanidine hydrochloride (458 mg, 4.8 mmol) was added in one portion to a stirred suspension of NaH (90 mg, 80% dispersion by wt in mineral oil, 2.97 mmol) in DME (10 mL) and the mixture was heated at 60° C. under N₂ for 30 min. A solution of ethyl (±)-cis-2-{[(1,4-dichloro-7-isoquinolinyl)sulphonyl]amino}cyclohexanecarboxylate (377 mg, 0.87 mmol) in DMA (5 mL) was added and the mixture heated at 90° C. for 4 h. The solvents were evaporated in vacuo, the residue was dissolved with EtOAc (200 mL), washed with aqueous NH₄Cl (20 mL), then with water (500 mL), and the combined aqueous washings were extracted with EtOAc (2×50 mL). The combined EtOAc extracts were washed with water (4×100 mL), dried (MgSO₄) and evaporated in vacuo. The residue was purified by column chromatography upon silica gel using using toluene-i-PrOH-0.880NH₃ (100:0:0 to 90:10:0.05) as eluant to give ethyl (±)-trans-2-{[(4-chloro-1-guanidino-7-isoquinolinyl)sulphonyl]amino}cyclohexanecarboxylate (65 mg, 0.14 mmol) as a white solid. [A small amount of ethyl (±)-cis-2-{[(4-chloro-1-guanidino-7-isoquinolinyl)sulphonyl]amino}cyclohexanecarboxylate (<20 mg) was also isolated.]

¹H (CDCl₃, 400 MHz) δ 1.1-1.8 (6H, mm), 1.1 (3H, t), 1.9 (1H, m), 2.0 (1H, m), 2.25 (1H, td), 3.45 (1H, m), 3.8-4.0 (2H, m), 8.05 (1H, d), 8.1 (1H, d), 8.15 (1H, s), 9.3 (1H, s) ppm.

LRMS 454, 456 (MH⁺).

### Example 47

### (a) t-Butyl cis-4-{[(4-chloro-1-guanidino-7-isoquinolinyl)sulphonyl]amino}cyclohexane-carboxylate

### (b) t-butyl trans-4-{[(4-chloro-1-guanidino-7-isoquinolinyl)sulphonyl]amino}cyclohexane-carboxylate

### (c) cis-4-{[(4-Chloro-1-guanidino-7-isoquinolinyl)sulphonyl]amino}cyclohexanecarboxylic acid hydrochloride

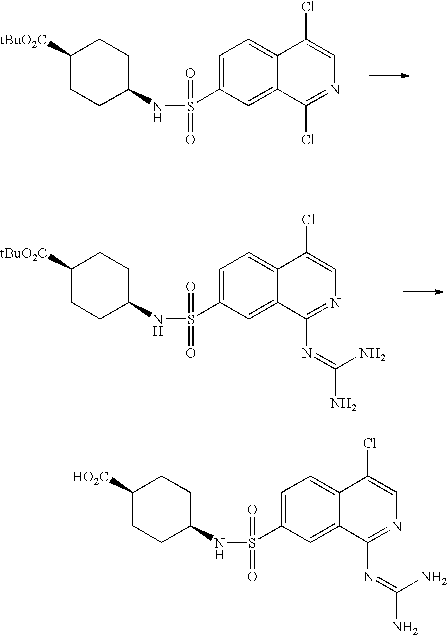

Guanidine hydrochloride (286 mg, 3.0 mmol) was added in one portion to a stirred suspension of NaH (56 mg, 80% dispersion by wt in mineral oil, 1.82 mmol) in DME (5 mL) and the mixture was heated at 60° C. under N₂ for 30 min. A solution of t-butyl cis-4-{[(1,4-dichloro-7-isoquinolinyl)sulphonyl]amino}cyclohexanecarboxylate (346 mg, 0.75 mmol) in DME (15 mL) was added and the mixture heated at 90° C. for 2 h. A second portion of guanidine (0.75 mmol)[prepared from guanidine hydrochloride (72 mg) and NaH (22 mg)] in DME (5 mL) was added and the mixture heated at 90° C. for 1 h. DMA (10 mL) was then added to the heterogeneous reaction mixture and the now homogeneous mixture heated for an additional 6 h. The solvents were evaporated in vacuo, the residue was quenched aqueous NH₄Cl (10 mL), diluted with water (150 mL) and extracted with EtOAc (2×150 mL). The combined organic extracts were washed with water (100 mL), dried (MgSO₄) and evaporated in vacuo. The residue was purified by repeated column chromatography upon silica gel using (i), pentane-EtOAc (100:0 to 25:75) and then (ii), PhMe-EtOAc (50:50 to 0:100) as eluant to give t-butyl cis-4-{[(4-chloro-1-guanidino-7-isoquinolinyl)sulphonyl]amino)cyclohexanecarboxylate (247 mg, 0.51 mmol). [A small amount of t-butyl trans-4-[(4-chloro-1-guanidino-7-isoquinolinyl)sulphonyl]amino}cyclohexanecarboxylate (20 mg) was also isolated.]

¹H (CDCl₃, 400 MHz) δ 1.4 (9H, s), 1.5-1.8 (8H, mm), 2.3 (1H, m), 3.4 (1H, m), 4.8-4.9 (1H, br), 6.1-6.55 (4H, br), 8.05 (1H, d), 8.1 (1H, d), 8.15 (1H, s), 9.3 (1H, s) ppm.

LRMS 482 (MH⁺), 963 (M₂H⁺).

Anal. Found: C, 52.14; H, 5.92; N, 14.19. Calc for C₂₁H₂₈ClN₅O₄S: C, 52.33; H, 5.86; N, 14.53.

t-Butyl cis-4-{[(4-chloro-1-guanidino-7-isoquinolinyl)sulphonyl]amino}cyclohexanecarboxylate (55 mg, 0.121 mmol) was suspended in a solution of EtOAc saturated with HCl (50 mL) and the mixture heated at reflux. The mixture was cooled, the white solid was collected by filtration, with EtOAc washing, and then dried to give cis-4-{[(4-chloro-1-guanidino-7-isoquinolinyl)sulphonyl]amino}-cyclohexanecarboxylic acid hydrochloride (110 mg, 0.236 mmol).

mp 287-289° C.

¹H (CDCl₃, 400 MHz) δ 1.5-1.6 (6H, m), 1.8-1.9 (2H, m), 2.35 (1H, m), 3.4 (1H, m), 8.35 (1H, d), 8.45 (1H, s), 8.5 (1H, d), 8.9 (1H, s) ppm

Anal. Found: C, 43.88; H, 4.61; N, 14.69. Calc for C₁₇H₂₀ClN₅O₄S.1.0HCl.0.2H₂O: C, 43.83; H, 4.63; N, 15.03.

### Example 48

### (a) Ethyl trans-4-{[(4-chloro-1-guanidino-7-isoquinolinyl)sulphonyl]amino}cyclohexane-carboxylate

### (b) trans-4-{[(4-Chloro-1-guanidino-7-isoquinolinyl)sulphonyl]amino}cyclohexanecarboxylic acid hydrochloride

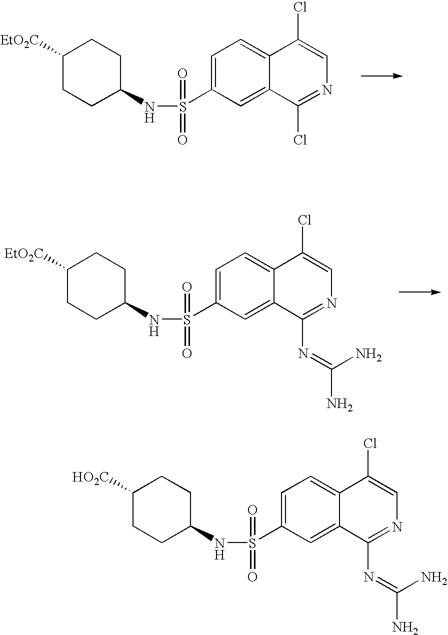

Guanidine hydrochloride (273 mg, 2.86 mmol) was added in one portion to a stirred suspension of NaH (55 mg, 80% dispersion by wt in mineral oil, 1.82 mimmol) in DME (10 mL) and the mixture was heated at 60° C. under N₂ for 30 min. A solution of ethyl trans-4-{[(1,4-dichloro-7-isoquinolinyl)sulphonyl]amino}cyclohexanecarboxylate (370 mg, 0.78 mmol) in DMA (10 mL) was added and the mixture heated at 90° C. for 3 h. The solvents were evaporated in vacuo, the residue was partitioned between Et₂O (100 mL), aqueous NH₄Cl (10 mL), and water (150 mL). The separated aqueous phase was extracted with Et₂O (3×100 mL) and the combined organic extracts were dried (MgSO₄) and evaporated in vacuo. The residue was purified by column chromatography upon silica gel using toluene-i-PrOH-0.880NH₃ (100:0:0 to 90:10:0.05) as eluant to give ethyl trans-4-{[(4-chloro-1-guanidino-7-isoquinolinyl)sulphonyl]amino}cyclohexanecarboxylate (70 mg, 0.15 mmol).

¹H (CDCl₃, 400 MHz) δ 1.1 (3H, s), 1.1-1.3 (4H, mm), 1.6 (2H, br d), 1.8 (2H, br d), 2.1 (1H, m), 2.9 (1H, m), 3.95 (2H, q), 7.1-7.4 (4H, br), 7.8 (1H, d), 8.0 (1H, d), 8.1 (1H, d), 8.1 (1H, s), 9.1 (1H, s) ppm.

LRMS 454,456 (MH⁺).

Anal. Found: C, 50.27; H, 5.56; N, 14.92. Calc for C₁₉H₂₄ClN₅O₄S: C, 50.27; H, 5.32; N, 15.43.

A solution of HCl (5 mL, 2 M, 10 mmol) was added to a solution of ethyl trans-4-{[(4-chloro-1-guanidino-7-isoquinolinyl)sulphonyl]amino}cyclohexanecarboxylate (55 mg, 0.121 mmol) in dioxane (5.0 mL) and the mixture was heated at reflux for 2 h. The solvents were evaporated in vacuo and the residue was purified by column chromatography upon MCl gel (CHP 20P) using water-MeOH (100:0 to 20:80) as eluant to give trans-4-{[(4-chloro-1-guanidino-7-isoquinolinyl)sulphonyl]amino}-cyclohexanecarboxylic acid. This material was dissolved in dilute HCl (20 mL, 0.1 M), the solvents were evaporated in vacuo, and the residue triturated with Et₂O to give trans-4-{[(4-chloro-1-guanidino-7-isoquinolinyl)sulphonyl]amino}cyclohexanecarboxylic acid hydrochloride (35 mg, 0.067 mmol) as a white solid.

mp>205° C. (dec).

¹H (CD₃OD, 400 MHz) δ 1.2-1.4 (4H, mm), 1.8 (2H, br d), 1.9 (2H, br d), 2.1 (1H, m), 3.1 (1H, m), 8.3 (1H, d), 8.45 (1H, s), 8.5 (1H, d), 8.9 (1H, s) ppm.

Anal. Found: C, 42.75; H, 5.04; N, 13.35. Calc for C₁₇H₂₀ClN₅O₄S.1.0HCl.1.5H₂O.0.4Et₂O: C, 43.04; H, 5.44; N, 13.49.

### Example 49

### (a) N-[(4-chloro-1-guanidino-7-isoquinolinyl)carbonyl]glycine t-butyl ester

### (b) N-[(4-Chloro-1-guanidino-7-isoquinolinyl)carbonyl]glycine trifluoroacetate

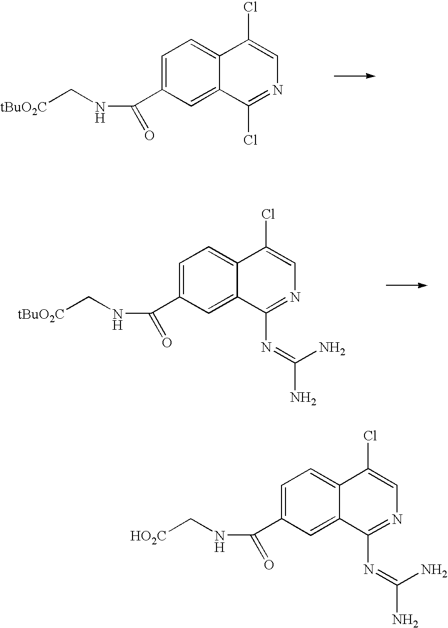

NaH (34 mg, 60% dispersion in mineral oil, 0.85 mmol) was added to a stirred solution of guandine hydrochloride (80 mg, 0.84 mmol) in DMSO (2 mL) at 23° C. After 30 min., N-[(1,4-dichloro-7-isoquinolinyl)carbonyl]glycine t-butyl ester (120 mg, 0.34 mmol) was added and the resultant solution heated at 90° C. for 21 h. After cooling, the mixture was poured into water (30 mL), extracted with EtOAc, and then with CH₂Cl₂, and the combined organic extracts were dried (Na₂SO₄) and evaporated in vacuo. The residue was purified by column chromatography on silica gel using CH₂Cl₂-MeOH-0.880NH₃ (90:10:1) as eluant to give N-[(4-chloro-1-guanidino-7-isoquinolinyl)carbonyl]glycine t-butyl ester (25 mg, 0.07 mmol) as a yellow gum.

LRMS 378 (MH⁺), 756 (M₂H⁺).

A solution of N-[(4-chloro-1-guanidino-7-isoquinolinyl)carbonyl]glycine t-butyl ester (24 mg, 0.06 mmol) in CF₃CO₂H (0.5 ml) was stirred at 0° C. for 1.5 h. The reaction mixture was diluted with PhMe, evaporated in vacuo, azeotroping with PhMe, and the residue triturated with Et₂O to give N-[(4-chloro-1-guanidino-7-isoquinolinyl)carbonyl]glycine trifluoroacetate (21 mg, 0.05 mmol) as a white solid.

mp>300° C.

¹H (TFA-d, 400 MHz) δ 4.6 (2H, s), 8.4 (1H, d), 8.45 (1H, s), 8.6 (1H, d), 9.3 (1H, s) ppm.

LRMS 322 (MH⁺).

Anal. Found: C, 40.60; H, 2.91; N, 15.47. Calc for C₁₃H₁₂ClN₅O₃.CF₃CO₂H: C, 40.58; H, 2.93; N, 15.46.

### Example 50

### (a) N-[(4-chloro-1-guanidino-7-isoquinolinyl)carbonyl]-β-alanine t-butyl ester

### (b) N-[(4-Chloro-1-guanidino-7-isoquinolinyl)carbonyl]-β-alanine

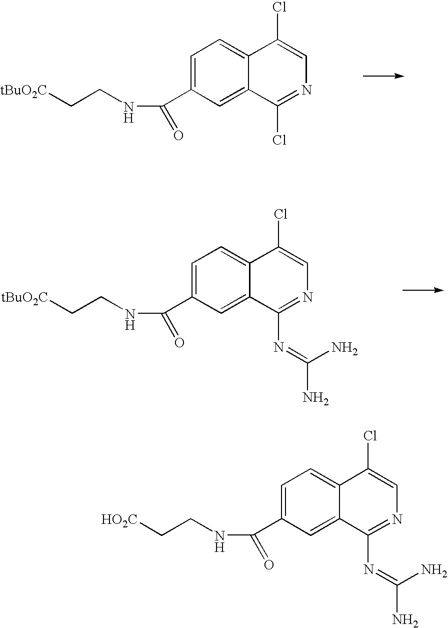

NaH (114 mg, 60% dispersion in mineral oil, 2.85 mmol) was added portionwise to a stirred solution of guanidine hydrochloride (272 mg, 2.85 mmol) in DMSO (8 mL) and the solution was heated at 80° C. for 20 min. N-[(1,4-Dichloro-7-isoquinolinyl)carbonyl]-p-alanine t-butyl ester (420 mg, 1.14 mmol) was added and the mixture heated at 90° C. overnight. The cooled mixture was poured into water, extracted with EtOAc, and the combined organic extracts were washed with water, saturated brine, dried (Na₂SO₄) and evaporayted in vacuo. The residue was crystallised from i-Pr₂O—CH₂Cl₂ to give N-[(4-chloro-1-guanidino-7-isoquinolinyl)carbonyl]-β-alanine t-butyl ester (190 mg, 0.48 mmol).

mp 224-226° C.

¹H (DMSO-d₆, 400 MHz) δ 1.4 (9H, s), 2.55-2.5 (2H, m), 3.5 (2H, dt), 7.0-7.3 (4H, br s), 7.85 (1H, d), 8.0 (1H, s), 8.1 (1H, d), 8.65 (1H, t), 9.1 (1H, s) ppm.

LRMS 392 (MH⁺), 783 (M₂H⁺).

Anal. Found: C, 54.89; H, 5.68; N, 17.94. Calc for C₁₈H₂₂ClN₅O₃: C, 55.17; H, 5.66; N, 17.87.

A solution of N-[(4-chloro-1-guanidino-7-isoquinolinyl)carbonyl]-β-alanine t-butyl ester (145 mg, 0.37 mmol) in CF₃CO₂H (1.5 mL) was stirred at 0° C. for 30 min, and then at room temperature for 1 h. PhMe (15 mL) was added, the mixture evaporated in vacuo, and the residue triturated with EtOAc and Et₂O to give N-[(4-Chloro-1-guanidino-7-isoquinolinyl)carbonyl]-β-alanine (117 mg, 0.26 mmol) as a white solid.

mp 235-236° C.

¹H (DMSO-d₆, 300 MHz) δ 2.6 (2H, t), 3.55 (2H, dt), 8.25 (1H, d), 8.35-8.4 (2H, m), 8.5 (4H, br s), 8.8-8.9 (2H, m) ppm.

LRMS 336 (MH⁺).

Anal. Found: C, 42.72; H, 3.56; N, 14.55. Calc for C₁₄H₁₄ClN₅O₂.0.25EtOAc: C, 42.75; H, 3.57; N, 14.49.

### Example 51

### (a) N-[(4-chloro-1-guanidino-7-isoquinolinyl)carbonyl]cycloleucine ethyl ester

### (b) N-[(4-Chloro-1-guanidino-7-isoquinolinyl)carbonyl]cycloleucine

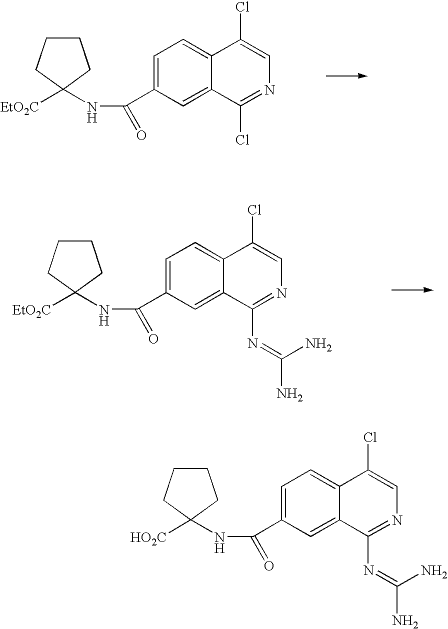

NaH (45 mg, 60% dispersion in mineral oil, 1.13 mmol) was added to t-BuOH and the mixture heated at 50° C. for 15 min. Guanidine hydrochloride (105 mg, 1.10 mmol) was added and the mixture heated at 50° C. for an additional 15 min. N-[(1,4-Dichloro-7-isoquinolinyl)carbonyl]cycloleucine ethyl ester (350 mg, 0.92 mmol) was added and the mixture heated at reflux for 17 h. The solvents were evaporated in vacuo and the residue purified by column chromatography on silica gel using CH₂Cl₂-MeOH-0.880NH₃ (90:10:1) as eluant, followed by trituration with CH₂Cl₂-i-Pr₂O, to give N-[(4-chloro-1-guanidino-7-isoquinolinyl)carbonyl]cycloleucine ethyl ester (55 mg, 0.14 mmol) as a pale yellow powder.

¹H (CDCl₃, 300 MHz) δ 1.0 (3H, t), 1.5-1.65 (4H, m), 1.8-2.0 (2H, m), 2.0-2.15 (2H, m), 3.9 (2H, q), 6.7 (4H, br s), 7.5 (1H, s), 7.7 (1H, d), 7.8 (1H, s), 7.9 (1H, d), 8.95 (1H, s) ppm.

LRMS 404 (MH⁺).

Anal. Found: C, 55.94; H, 5.42; N, 16.94. Calc for C₁₉H₂₂ClN₅O_{3.}0.25 H₂O: C, 55.87; H, 5.55; N, 17.14.

A partly heterogeneous solution of N-[(4-chloro-1-guanidino-7-isoquinolinyl)carbonyl]cycloleucine ethyl ester (45 mg, 0.11 mmol) in dioxane (1.5 mL) was stirred with aqueous NaOH (1 mL, 2 M) for 2.5 h at 23° C. Dilute HCl (1 mL, 2 M) was added to give a cream suspension. The solid was collected by filtration and dried in vacuo to yield N-[(4-chloro-1-guanidino-7-isoquinolinyl)carbonyl]cycloleucine (40 mg, 0.11 mmol).

mp>275° C.

¹H (TFA-d, 400 MHz) δ 1.9-2.1 (4H, m), 2.2-2.4 (2H, m), 2.5-2.7 (2H, m), 8.3 (1H, d), 8.35 (1H, s), 8.45 (1H, d), 9.25 (1H, s) ppm.

LRMS 376 (MH⁺), 751 (M₂H⁺).

Anal. Found: C, 51.67; H, 4.92; N, 17.39. Calc for C₁₇H₁₈ClN₅O₃.H₂O: C, 51.84; H, 5.11; N, 17.78.

### Example 52

### (a) N-[(4-chloro-1-guanidino-7-isoquinolinyl)carbonyl]-DL-phenylglycine t-butyl ester

### (b) N-[(4-Chloro-1-guanidino-7-isoquinolinyl)carbonyl]-DL-phenylglycine trifluoroacetate

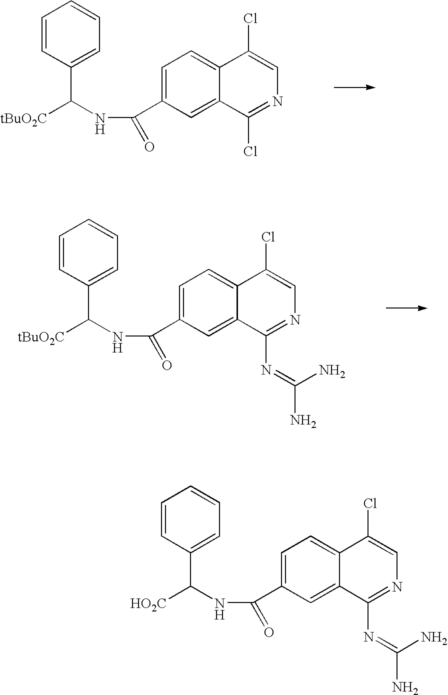

A mixture of guanidine hydrochloride (326 mg, 3.41 mmol) and NaH (137 mg, 60% dispersion in oil, 3.43 mmol) in DMSO (5 mL) was heated to 70° C., a solution of N-[(1,4-dichloro-7-isoquinolinyl)carbonyl]-DL-phenylglycine t-butyl ester (590 mg, 1.37 mmol) in DMSO (3 mL) was added, and the mixture heated at 80-90° C. overnight. After cooling, the reaction mixture was poured into water (50 mL) and extracted with EtOAc (3×30 mL). The combined organic extracts were washed with water, dried (Na₂SO₄), and evaporated in vacuo. Purification of the residue by column chromatography on silica gel using CH₂Cl₂-MeOH-0.880NH₃ (90:10:1) as eluant, followed by crystallisation from i-Pr₂O, gave N-[(4-chloro-1-guanidino-7-isoquinolinyl)carbonyl]-DL-phenylglycine t-butyl ester (110 mg, 0.24 mmol) as a pale yellow solid.

mp 158° C. (foam), 170° C. (dec).

¹H (CDCl₃, 300 MHz) δ 1.4 (9H, s), 5.7 (1H, d), 6.5 (4H, br s), 7.25-7.4 (3H, m), 8.05 (1H, d), 8.10 (1H, s), 8.15 (1H, d), 9.2 (1H, d) ppm.

LRMS 454 (MH⁺).

Anal. Found: C, 61.53; H, 5.96; N, 14.27. Calc for C₂₃H₂₄ClN₅O₃.0.3i-Pr₂O: C, 61.53; H, 5.92; N, 14.27.

A solution of N-[(4-chloro-1-guanidino-7-isoquinolinyl)carbonyl]-DL-phenylglycine t-butyl ester (100 mg, 0.22 mmol) in CF₃CO₂H (1.5 mL) was stirred at 0° C. for 30 min, and then at 23° C. for 1 h. The reaction mixture was diluted with PhMe (15 mL) and evaporated in vacuo. The residual gum was triturated with EtOAc, and then Et₂O, and the resulting white solid was dried in vacuo to give N-[(4-chloro-1-guanidino-7-isoquinolinyl)carbonyl]-DL-phenylglycine trifluoroacetate (50 mg, 0.10 mmol).

¹H (DMSO-d₆, 300 MHz) δ 5.6 (1H, d), 7.3-7.45 (3H, m), 7.55 (2H, d), 8.2 (1H, d), 8.2-8.4 (5H, m), 8.45 (1H, d), 8.95 (1H, s), 9.4 (1H, d) ppm.

LRMS 398 (MH⁺).

Anal. Found: C, 49.72; H, 3.68; N, 14.04. Calc for C₁₉H₁₆ClN₅O₃.0.95CF₃CO₂H: C, 49.27; H, 3.35; N, 13.68.

### Example 53

### (a) N-[(4-chloro-1-guanidino-7-isoquinolinyl)carbonyl]-L-phenylglycine t-butyl ester

### (b) N-[(4-Chloro-1-guanidino-7-isoquinolinyl)carbonyl]-L-phenylglycine trifluoroacetate

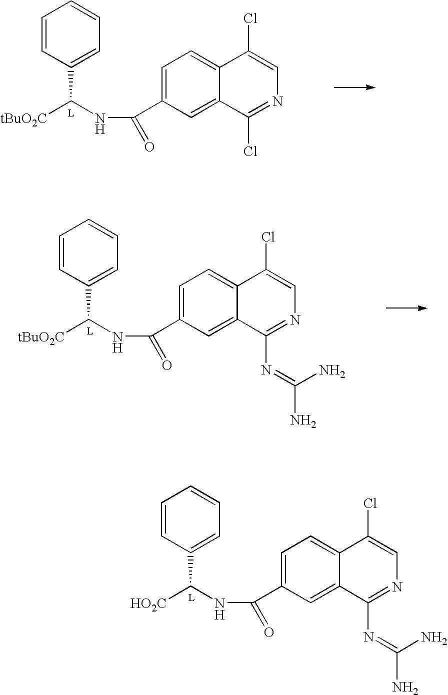

NaH (38 mg, 80% dispersion in mineral oil, 1.27 mmol) was added to a stirred solution of guanidine hydrochloride (121 mg, 1.27 mmol) in DMSO (4 mL) at 23° C., and the mixture heated at 80-85° C. for 15 min. N-[(1,4-Dichloro-7-isoquinolinyl)carbonyl]-L-phenylglycine t-butyl ester (218 mg, 0.51 mmol) was added and the mixture heated at 85° C. for 4 h. The cooled solution was poured into water and extracted with EtOAc (×3). The combined organics were washed with saturated brine, dried (Na₂SO₄) and evaporated in vacuo. The residue was crystallised with i-Pr₂O to give N-[(4-chloro-1-guanidino-7-isoquinolinyl)carbonyl]-L-phenylglycine t-butyl ester (55 mg, 0.12 mmol) as a pale yellow solid.

mp 147° C. (dec).

¹H (CDCl₃, 400 MHz) δ 1.4 (9H, s), 5.7 (1H, d), 6.2-6.8 (4H, br s), 7.3-7.4 (3H, m), 7.45-7.5 (3H, m), 8.0-8.1 (2H, m), 8.15-8.2 (1H, d), 9.2 (1H, s) ppm.

LRMS 454 (MH⁺), 907 (M₂H⁺).

Anal. Found: C, 61.22; H, 6.01; N, 13.91. Calc for C₂₃H₂₄ClN₅O₃.0.4i-Pr₂O: C, 61.21; H, 6.07; N, 14.05.

A solution of N-[(4-chloro-1-guanidino-7-isoquinolinyl)carbonyl]-L-phenylglycine t-butyl ester (40 mg, 0.09 mmol) in CF₃CO₂H (1 mL) was stirred at room temperature for 1 h. The reaction mixture was diluted with PhMe, evaporated in vacuo, and the residue triturated with EtOAc to give N-[(4-chloro-1-guanidino-7-isoquinolinyl)carbonyl]-L-phenylglycine trifluoroacetate (32 mg, 0.06 mmol) as a white powder.

mp 163° C. (shrinks), >200° C. (dec).

¹H (TFA-d, 400 MHz) δ 5.85 (1H, s), 7.35-7.4 (3H, m), 7.4-7.45 (2H, m), 8.25 (1H, d), 8.3 (1H, s), 8.4 (1H, d), 9.15 (1H, s) ppm.

LRMS 398 (MH⁺), 795 (M₂H⁺).

Anal. Found: C, 48.28; H, 3.74; N, 13.57. Calc for C₁₉H₁₆ClN₅O₃.CF₃CO₂H.0.5H₂O: C, 48.43; H, 3.48; N, 13.45.

### Example 54

### (a) N-[(4-chloro-1-guanidino-7-isoquinolinyl)carbonyl]-D-phenylglycine t-butyl ester

### (b) N-[(4-Chloro-1-guanidino-7-isoquinolinyl)carbonyl]-D-phenylglycine trifluoroacetate

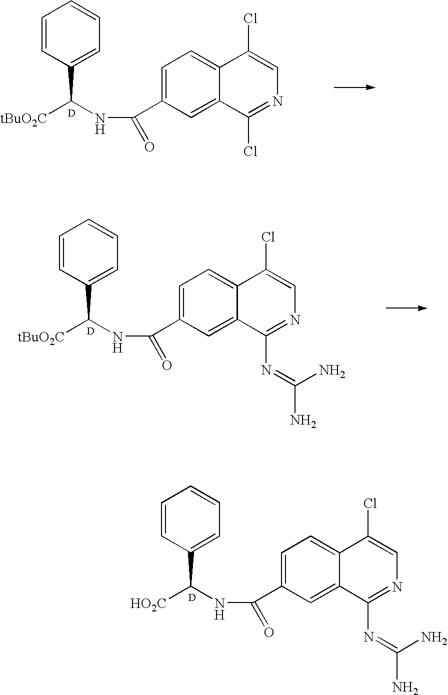

NaH (30 mg, 80% dispersion in mineral oil, 1.0 mmol) was added to a solution of guanidine hydrochloride (97 mg, 1.0 mmol) in DMSO (3 mL) and the solution heated to 80° C. for 30 min. N-[(1,4-Dichloro-7-isoquinolinyl)carbonyl]-D-phenylglycine t-butyl ester (175 mg, 0.41 mmol) was added, the mixture heated at 85° C. for 3.5 h, and then at 23° C. overnight. The mixture was poured into water (25 mL), extracted with EtOAc (3×20 mL), and the combined organics washed with brine, dried (MgSO₄), and evaporated in vacuo. The reside was purified by column chromatography on silica gel using CH₂Cl₂-MeOH-0.880NH₃ (95:5:0.5) as eluant, followed by crystallisation from CH₂Cl₂i-Pr₂O, to give N-[(4-chloro-1-guanidino-7-isoquinolinyl)carbonyl]-D-phenylglycine t-butyl ester (37 mg, 0.08 mmol) as a solid.

mp 154-156° C. (dec).

¹H (CDCl₃, 400 MHz) δ 1.4 (9H, s), 5.7 (1H, d), 7.3-7.4 (3H, m), 7.4-7.5 (3H, m), 8.05 (1H, d), 8.05 (1H s), 8.15 (1H, d), 9.2 (1H, s) ppm.

LRMS 454 (MH⁺), 907 (M₂H⁺).

Anal. Found: C, 61.15; H, 6.00; N, 13.87. Calc for C₂₃H₂₄ClN₅O₃.0.45i-Pr₂O.0.2 H₂O: c, 61.31; H, 6.15; N, 13.91.

A solution of N-[(4-chloro-1-guanidino-7-isoquinolinyl)carbonyl]-D-phenylglycine t-butyl ester (40 mg, 0.09 mmol) in CF₃CO₂H (0.5 mL) was stirred at room temperature for 1 h. The solution was diluted with PhMe, evaporated in vacuo, and the residue was triturated with Et₂O to give N-[(4-chloro-1-guanidino-7-isoquinolinyl)carbonyl]-D-phenylglycine trifluoroacetate (21 mg, 0.04 mmol) as a white powder.

mp 222° C. (dec).

¹H (TFA-d, 400 MHz) δ 5.9 (1H, s), 7.4-7.5 (3H, m), 7.5-7.55 (2H, m), 8.3 (1H, d), 8.35 (1H, s), 8.4 (1H, d), 9.2 (1H, s) ppm.

LRMS 398 (MH⁺), 795 (M₂H⁺).

Anal. Found: C, 49.02; H, 3.42; N, 13.26. Calc for C₁₉H₁₆ClN₅O₃.CF₃CO₂H.0.25H₂O: C, 48.85; H, 3.42; N, 13.56.

### Example 55

### (a) N-[(4-chloro-1-guanidino-7-isoquinolinyl)carbonyl]-DL-valine t-butyl ester

### (b) N-[(4-Chloro-1-guanidino-7-isoquinolinyl)carbonyl]-DL-valine trifluoroacetate

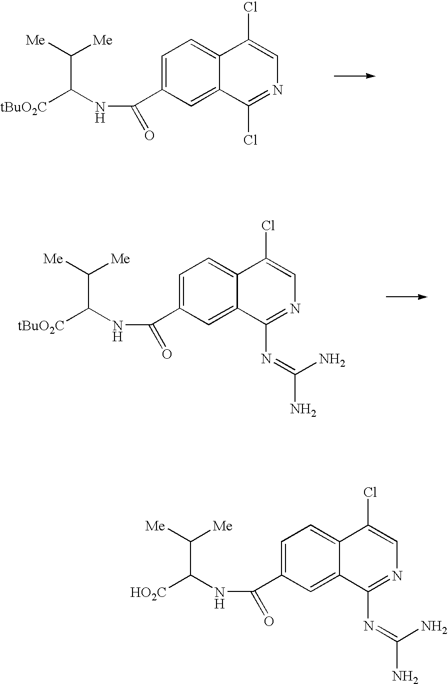

NaH (88 mg, 60% dispersion in mineral oil, 2.2 mmol) was added to a stirred solution of guanidine hydrochloride (210 mg, 2.2 mmol) in DMSO (5 mL) at 70° C. and the solution stirred for 30 min. N-[(1,4-Dichloro-7-isoquinolinyl)carbonyl]-DL-valine t-butyl ester (350 mg, 0.88 mmol) was added and the solution heated at 80-90° C. overnight. The cooled mixture was poured into water, extracted with EtOAc (3×20 mL), and the combined organic extracts were dried (MgSO₄) and evaporated in vacuo.

The residue was crystallised with CH₂Cl₂-i-Pr₂O to give N-[(4-chloro-1-guanidino-7-isoquinolinyl)carbonyl]-DL-valine t-butyl ester (285 mg, 0.68 mmol) as a yellow solid.

mp 178-180° C. (dec).

¹H (CDCl₃, 300 MHz) shows 1:1 mixture of rotamers, δ 1.0 (½ of 6H, d), 1.05 (½ of 6H, d), 1.5 (9H, s), 2.2-2.4 (1H, m), 4.7 (½ of 1H, d), 4.75 (½ of 1H, d), 6.2-6.8 (4H, br s), 6.9 (1H, d), 8.05 (1H, d), 8.1 (1H, s), 8.15 (1H, d), 9.1 (1H, s) ppm.

LRMS 420 (MH⁺), 839 (M₂H⁺).

Anal. Found: C, 56.00; H, 6.35; N, 16.33. Calc for C₂₀H₂₆ClN₅O₃.0.5H₂O: C, 55.71; H, 6.36; N, 16.32.

A solution of N-[(4-Chloro-1-guanidino-7-isoquinolinyl)carbonyl]-DL-valine t-butyl ester (200 mg, 0.48 mmol) in CF₃CO₂H (1.5 mL) was stirred at 0° C. for 30 min, and at 23° C. for 1 h. The reaction mixture was diluted with PhMe, evaporated in vacuo, and the residue triturated with EtOAc to give N-[(4-chloro-1-guanidino-7-isoquinolinyl)carbonyl]-DL-valine trifluoroacetate (170 mg, 0.36 mmol) as a white solid.

mp 243-245° C. (dec).

¹H (DMSO-d₆, 300 MHz) shows a 1:1 mixture of rotamers, δ 0.95 (½ of 6H, d), 1.0 (½of 6H, d), 2.15-2.3 (1H, m), 4.35 (1H, t), 8.25 (1H, d), 8.4 (1H, s), 8.45 (1H, d), 8.4-8.6 (4H, br s), 8.85 (1H, d), 8.9 (1H, s) ppm.

LRMS 364 (MH⁺).

Anal. Found: C, 44.96; H, 3.95; N, 14.56. Calc for C₁₆H₁₆ClN₅O₃.CF₃CO₂H: C, 45.24; H, 4.01; N, 14.65.

### Example 56

### (a) N-[(4-chloro-1-guanidino-7-isoquinolinyl)carbonyl]-DL-proline t-butyl ester

### (b) N-[(4-Chloro-1-guanidino-7-isoquinolinyl)carbonyl]-DL-proline trifluoroacetate

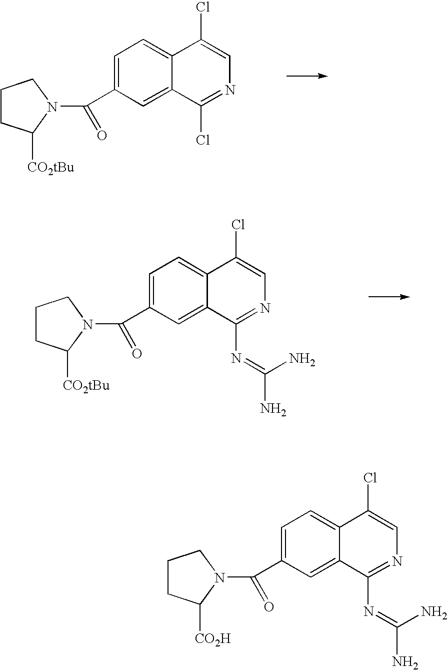

NaH (65 mg, 60% dispersion in mineral oil, 1.63 mmol) was added to a stirred solution of guanidine hydrochloride (154 mg, 1.61 mmol) in DMSO (5 mL) at 50° C. and the solution stirred for 15 min. N-[(1,4-Dichloro-7-isoquinolinyl)carbonyl]-DL-proline t-butyl ester (253 mg, 0.64 mmol) was added and the mixture was heated at 80° C. overnight. The mixture was poured into water (20 mL) and extracted with EtOAc (×2). The combined organic extracts were washed with water, brine, dried over (MgSO₄), and evaporated in vacuo. The residue was crystallised with CH₂Cl₂-i-Pr₂O to give N-[(4-chloro-1-guanidino-7-isoquinolinyl)carbonyl]-DL-proline t-butyl ester (241 mg, 0.58 mmol).

mp 147-149° C. (dec).

¹H (CDCl₃, 300 MHz) shows 1:3 mixture of rotamers, δ 1.55 (9H, s), 1.8-2.1 (3H, m), 2.15-2.45 (1H, m), 3.55-3.65 (1H, m), 3.75-3.85 (1H, m), 4.35-4.45 (1H, m), 6.5-7.2 (4H, br m), 7.7 (¼ of 1H, d), 7.85 (¾ of 1H, d), 7.9-8.1 (2H, m), 8.85 (¼ of 1H, s), 8.95 (¾ of 1H, s) ppm.

LRMS 418 (MH⁺), 835 (M₂H⁺).

Anal. Found: C, 58.46; H, 6.49; N, 14.95. Calc for C₂₀H₂₄ClN₅O₃.0.4i-Pr₂O: C, 58.65; H, 6.50; N, 15.27.

A solution of N-[(4-Chloro-1-guanidino-7-isoquinolinyl)carbonyl]-DL-proline t-butyl ester (175 mg, 0.42 mmol) in CF₃CO₂H (1 mL) was stirred at room temperature for I h. The solution was diluted with PhMe, evaporated in vacuo, and the residue was triturated with Et₂O to give N-[(4-chloro-1-guanidino-7-isoquinolinyl)carbonyl]-DL-proline trifluoroacetate (156 mg, 0.33 mmol) as a white solid.

mp 185° C. (dec).

¹H (DMSO-d₆+1 drop TFA-d, 300 MHz) δ 1.8-2.1 (3H, m), 2.25-2.4 (1H, m), 3.45-3.7 (2H, m), 4.4-4.5 (1H, m), 8.0-8.6 (4H, m) ppm.

LRMS 362 (MH⁺).

Anal. Found: C, 45.65; H, 3.84; N, 14.43. Calc for C₁₆H₁₆ClN₅O₃.CF₃CO₂H: C, 45.43; H, 3.60; N, 14.72.

### Example 57

### (a) N-[(4-chloro-1-guanidino-7-isoquinolinyl)carbonyl]-DL-phenylalanine t-butyl ester

### (b) N-[(4-Chloro-1-guanidino-7-isoquinolinyl)carbonyl]-DL-phenylalanine trifluoroacetate

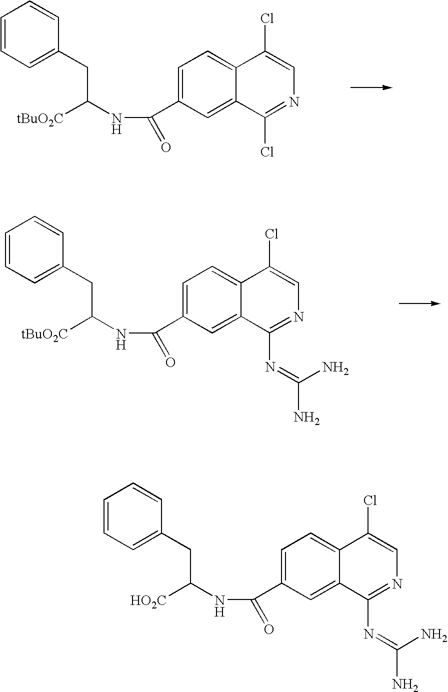

NaH (78 mg, 60% dispersion in mineral oil, 1.95 mmol) was added to a solution of guanidine hydrochloride (188 mg, 1.97 mmol) in DMSO (6 mL) at 50° C. and the solution was stirred for 15 min. N-[(1,4-Dichloro-7-isoquinolinyl)carbonyl]-DL-phenylalanine t-butyl ester (350 mg, 0.79 mmol) was added and the mixture heated at 80° C. overnight. The cooled mixture was poured into water (50 mL) and extracted with EtOAc (2×25 mL). The combined organics were washed with brine, dried (Na₂SO₄) and evaporated in vacuo. The residue was crystallised with CH₂Cl₂-i-Pr₂O to give N-[(4-chloro-1-guanidino-7-isoquinolinyl)carbonyl]-DL-phenylalanine i-butyl ester (172 mg, 0.37 mmol) as a cream coloured solid.

mp 201-203° C. (dec).

¹H (CDCl₃, 300 MHz) δ 1.45 (9H, s), 1.5-1.8 (1H, br m), 3.25 (2H, d), 5.0 (1H, dt), 6.0-6.8 (3H, br s), 6.9 (1H, d), 7.15-7.35 (5H, m), 8.0-8.1 (3H, m), 9.1 (1H, s) ppm.

LRMS 468 (MH⁺), 935 (M₂H⁺).

Anal. Found: C, 61.60; H, 5.60; N, 14.97. Calc for C₂₄H₂₆ClN₅O₃: C, 61.60; H, 5.76; N, 14.68.

A solution of N-[(4-Chloro-1-guanidino-7-isoquinolinyl)carbonyl]-DL-phenylalanine t-butyl ester (210 mg, 0.48 mmol) in CF₃CO₂H (1 mL) was stirred at room temperature for 1 h. The solution was diluted with PhMe, evaporated in vacuo, and the residue was triturated with Et₂O to give N-[(4-chloro-1-guanidino-7-isoquinolinyl)carbonyl]-DL-phenylalanine trifluoroacetate (196 mg, 0.37 mmol).

mp 192° C. (dec).

¹H (DMSO-d₆+1 drop TFA-d, 300 MHz) δ 3.1 (1H, dd), 3.25 (1H, dd), 4.7 (1H, dd), 7.1-7.35 (5H, m), 8.25 (1H, d), 8.35 (1H, s), 8.35 (1H, d), 8.9 (1H, s), 9.15 (½H, d partially exchanged amide NH) ppm.

LRMS 412 (MH⁺).

Anal. Found: C, 50.92; H, 3.81; N, 13.57. Calc for C₂₀H₁₈ClN₅O₃.0.9CF₃CO₂H: C, 50.90; H, 3.70; N, 13.61.

### Example 58

### (a) N-[(4-chloro-1-guanidino-7-isoquinolinyl)carbonyl]-DL-leucine t-butyl ester

### (b) N-[(4-Chloro-1-guanidino-7-isoquinolinyl)carbonyl]-DL-leucine trifluoroacetate

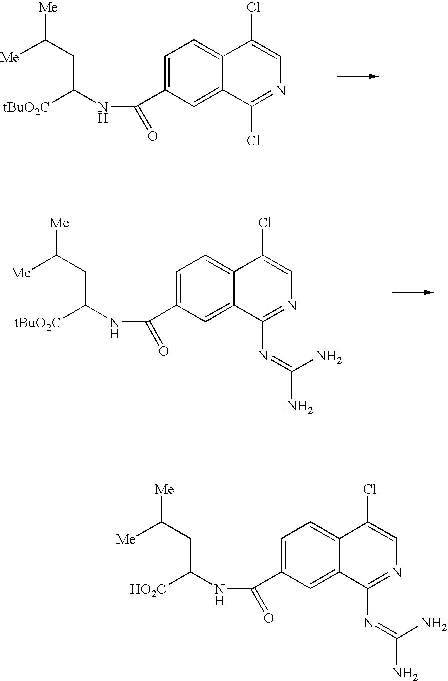

NaH (73 mg, 60% dispersion in mineral oil, 1.83 mmol) was added to a stirred solution of guanidine hydrochloride (174 mg, 1.82 mmol) in DMSO (6 mL) at 50° C. and the solution stirred for 15 min N-[(1,4-Dichloro-7-isoquinolinyl)carbonyl]-DL-leucine t-butyl ester (300 mg, 0.73 mmol) was added and the solution heated at 80° C. overnight. The cooled mixture was poured into water (50 mL), extracted with EtOAc (2×25 mL) and the combined organic extracts were washed with brine, dried (Na₂SO₄) and evaporated in vacuo. The residue was crystallised with CH₂Cl₂-i-Pr₂O to give N-[(4-chloro-1-guanidino-7-isoquinolinyl)carbonyl]-DL-leucine t-butyl ester (185 mg, 0.43 mmol).

mp 210-212° C. (dec).

¹H (CDCl₃, 300 MHz) 67 0.9-1.0 (6H, m), 1.5 (9H, s), 1.6-1.8 (3H, m), 4.7-4.8 (1H, m), 6.4-7.0 (4H, br s), 6.85 (1H, d), 8.05 (1H, d), 8.05 (1H, s), 8.15 (1H, d), 9.15 (1H, s) ppm.

LRMS 434 (MH⁺), 866 (M₂H⁺).

Anal. Found: C, 58.35; H, 6.75; N, 15.51. Calc for C₂₁H₂₈ClN₅O₃.0.15i-Pr₂O: C, 58.55; H, 6.75; N, 15.59.

A solution of N-[(4-chloro-1-guanidino-7-isoquinolinyl)carbonyl]-DL-leucine t-butyl ester (184 mg, 0.57 mmol) in CF₃CO₂H (1 mL) was stirred at room temperature for I h. The solution was diluted with PhMe, evaporated in vacuo, and the residue was triturated with Et₂O to give N-[(4-chloro-1-guanidino-7-isoquinolinyl)carbonyl]-DL-leucine trifluoroacetate (183 mg, 0.37 mmol).

mp 249° C.

¹H (DMSO-d₆, 300 MHz) 1:1 mixture of rotamers, δ 0.9 (½ of 6H, d), 0.95 (½ of 6H, d), 1.6-1.8 (3H, m), 4.45-4.5 (1H, m), 8.35 (1H, d), 8.4 (1H, s), 8.4 (1H, d), 8.3-8.6 (4H, br s), 8.95 (1H, s), 9.0 (1H, d) ppm.

LRMS 378 (MH⁺).

Anal. Found: C, 46.31; H, 4.27; N, 14.08. Calc for C₁₇H₂₀ClN₃O₃.CF₃CO₂H: C, 46.39; H, 4.30; N, 14.24.

### Example 59

### (a) t-butyl DL-3-{[(4-chloro-1-guanidino-7-isoquinolinyl)carbonyl]amino}-3-phenylpropanoate

### (b) DL-3-{[(4-Chloro-1-guanidino-7-isoquinolinyl)carbonyl]amino}-3-phenylpropanoic acid trifluoroacetate

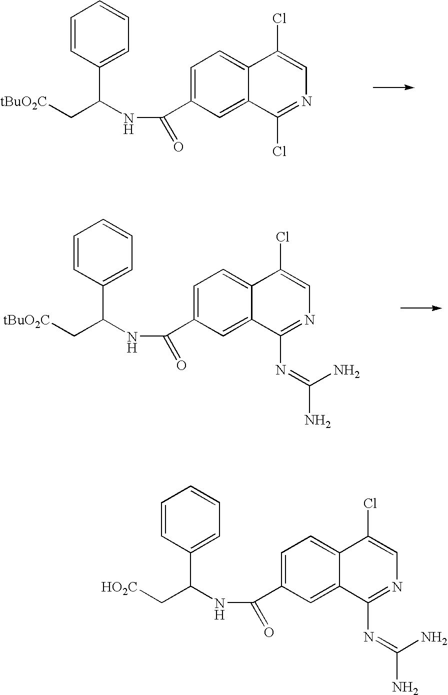

NaH (67 mg, 60% dispersion in oil, 1.68 mmol) was added to a solution of guanidine hydrochloride (161 mg, 1.69 mmol) in DMSO (6 mL) and the solution was heated to 50° C. for 15 mins. t-Butyl DL-3-[(1,4-dichloro-7-isoquinolinyl)carbonyl]amino}-3-phenylpropanoate (300 mg, 0.67 mmol) was added and the mixture heated at 80° C. overnight. The cooled mixture was poured into water (50 mL) and extracted with EtOAc (2×25 mL). The combined organic extracts were washed with brine, dried (Na₂SO₄) and evaporated in vacuo. The residue was crystallised with i-Pr₂O to give t-butyl DL-3-{[(4-chloro-1-guanidino-7-isoquinolinyl)carbonyl]amino}-3-phenylpropanoate (55 mg, 0.12 mmol) as a yellow solid.

mp 227° C. (dec).

¹H (CDCl₃+drop of DMSO-d₆, 300 MHz) δ 1.25 (9H, s), 2.75 (1H, dd), 2.85 (1H, dd), 5.5 (1H, dd), 6.4-6.8 (4H, br s), 7.1-7.35 (5H, m), 7.8 (1H, d), 7.9 (1H, d), 7.95 (1H, s), 8.05 (1H, d), 9.05 (1H, s) ppm.

LRMS 468 (MH⁺).

Anal. Found: C, 61.48; H, 5.62; N, 14.70. Calc for C₂₄H₂₆ClN₅O₃: C, 61.60; H, 5.60; N, 14.97.

A solution of t-butyl DL-3-{[(4-chloro-1-guanidino-7-isoquinolinyl)carbonyl]amino}-3-phenylpropanoate (153 mg, 0.33 mmol) in CF₃CO₂H (1 mL) was stirred at room temperature for 1 h. The solution was diluted with PhMe, evaporated in vacuo, and the residue was triturated with Et₂O to give DL-3-{[(4-chloro-1-guanidino-7-isoquinolinyl)carbonyl]amino)-3-phenylpropanoic acid trifluoroacetate (132 mg, 0.25 mmol).

mp. 241-244° C.

¹H (DMSO-d₆+1 drop TFA-d, 300 MHz) δ 2.8 (1H, dd), 2.95 (1H, dd), 5.5-5.6 (1H, m), 7.2-7.35 (3H, m), 7.4 (2H, d), 8.25 (1H, d), 8.35 (1H, s), 8.4 (1H, d), 8.9 (1H, s) ppm.

LRMS 412 (MH⁺).

Anal. Found: C, 49.95; H, 3.64; N, 13.03. Calc for C₂₀H₁₈ClN₅O₃.CF₃CO₂H: C, 50.25; H, 3.45 N, 13.32.

### Example 60

### (a) N-[(4-chloro-1-guanidino-7-isoquinolinyl)carbonyl]-DL-aspartic acid α,β-di-t-butyl ester

### (b) N-[(4-Chloro-1-guanidino-7-isoquinolinyl)carbonyl]-DL-aspartic acid trifluoroacetate

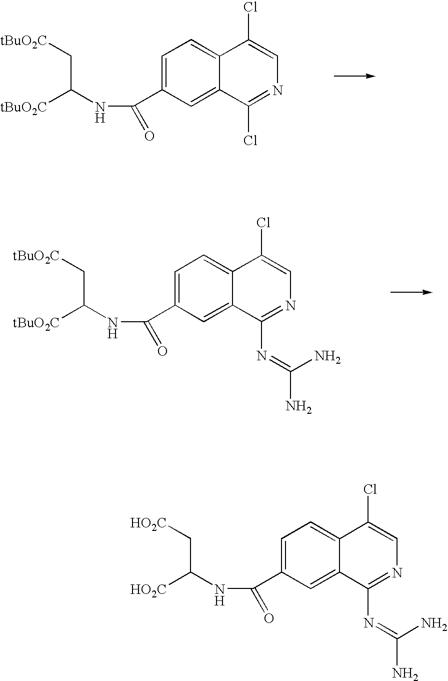

NaH (53 mg, 80% dispersion in mineral oil, 1.77 mmol) was added to a solution of guanidine hydrochloride (168 mg, 1.76 mmol) in DMSO (6 mL) and the solution ws heated to 50° C. for 30 min. N-[(1,4-Dichloro-7-isoquinolinyl)carbonyl]-DL-aspartic acid a,p-di-t-butyl ester (330 mg, 0.70 mmol) was added and the mixture heated at 80-90° C. overnight. The cooled mixture was poured into water (50 mL) and extracted with EtOAc extract (5×20 mL). The combined organic extracts were washed with water, brine, dried (Na₂SO₄) and evaporated in vacuo. The residue was purified by (i), trituration with i-Pr₂O (ii), column chromatography on silica gel using CH₂Cl₂-MeOH-0.880NH₃ (95:5:0.5) as eluant, and (iii), crystallisation from i-Pr₂O, to give N-[(4-chloro-1-guanidino-7-isoquinolinyl)carbonyl]-DL-aspartic acid α,β-di-t-butyl ester (145 mg, 0.29 mmol) as a yellow solid.

mp 165-167° C.

¹H (CDCl₃, 300 MHz) δ 1.45 (9H, s), 1.5 (9H, s), 2.9 (1H, dd), 3.0 (1H, dd), 4.95-5.0 (1H, m), 7.5 (1H, d), 7.95 (1H, s), 8.0 (1H, d), 8.15 (1H, d), 9.2 (1H, s) ppm.

LRMS 492 (MH⁺) 983 (M₂H⁺).

Anal. Found: C, 56.06; H, 6.28; N, 13.92. Calc for C₂₃H₂₀ClN₅O₅: C, 56.15; H, 6.15; N, 14.24.

A solution of N-[(4-chloro-1-guanidino-7-isoquinolinyl)carbonyl]-DL-aspartic acid α,β-di-t-butyl ester (120 mg, 0.24 mmol) in CF₃CO₂H (1 mL) was stirred at room temperature for 1 h. The solution was diluted with PhMe, evaporated in vacuo, and the residue was triturated with Et₂O to give N-[(4-chloro-1-guanidino-7-isoquinolinyl)carbonyl]-DL-aspartic acid trifluoroacetate (60 mg, 0.12 mmol).

mp 125° C. (dec).

¹H (TFA-d, 400 MHz) δ 3.3-3.4 (2H, m), 5.35-5.4 (1H, m), 8.25 (1H, d), 8.3 (1H, s), 8.45 (1H, d), 9.2 (1H, s) ppm.

LRMS 380 (MH⁺), 758 (M₂H⁺).

Anal. Found: C, 43.22; H, 3.75; N, 14.31. Calc for C₁₅H₁₄ClN₅O₅.0.8CF₃CO₂H.0.25Et₂O: C, 43.19; H, 3.56; N, 14.31.

### Example 61

### (a) O-t-butyl-N-[(4-chloro-1-guanidino-7-isoquinolinyl)carbonyl]-DL-serine t-butyl ester

### (b) N-[(4-Chloro-1-guanidino-7-isoquinolinyl)carbonyl]-DL-serine trifluoroacetate

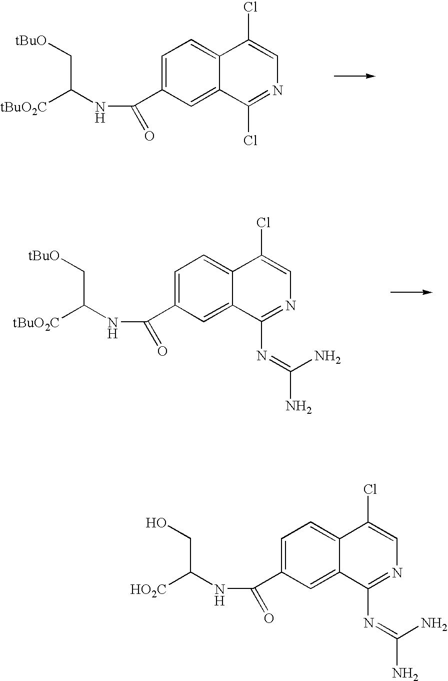

NaH (54 mg, 80% dispersion in mineral oil, 1.80 mmol) was added to a solution of guanidine hydrochloride (173 mg, 1.81 mmol) in DMSO (6 mL) and the solution was heated to 80° C. for 30 min. O-t-Butyl-N-[(1,4-dichloro-7-isoquinolinyl)carbonyl]-DL-serine t-butyl ester (330 mg, 0.70 mmol) was added and the mixture heated at 80° C. for 3 h. The cooled mixture was poured into water (50 mL) and extracted with EtOAc. The combined organic extracts were washed with water, brine, dried (Na₂SO₄) and evaporated in vacuo. The residue was crystallised with i-Pr₂O to give O-t-butyl-N-[(4-chloro-1-guanidino-7-isoquinolinyl)carbonyl]-DL-serine 1-butyl ester (138 mg, 0.30 mmol) as a yellow solid.

mp 215-219° C.

¹H (CDCl₃, 300 MHz) δ 1.2 (9H, s), 1.5 (9H, s), 1.5-1.7 (1H, br s), 3.75 (1H, dd), 3.95 (1H, dd), 4.8-4.9 (1H, m), 6.2-6.8 (3H, br s), 7.25-7.3 (1H, m), 8.0 (1H, s), 8.05 (1H, d), 8.15 (1H, d), 9.2 (1H, s) ppm.

LRMS 464 (MH⁺), 927 (M₂H⁺).

Anal. Found: C, 56.88; H, 6.65; N, 15.10. Calc for C₂₂H₃ClN₅O₄.0.25H₂O.0.2i-Pr₂O: C, 57.00; H, 6.87; N, 14.32.

A solution of O-t-butyl-N-[(4-chloro-1-guanidino-7-isoquinolinyl)carbonyl]-DL-serine t-butyl ester in CF₃CO₂H (1 mL) was stirred at room temperature for 1 h. The solution was diluted with PhMe, evaporated in vacuo, and the residue was recystallised twice from MeOH-EtOAc to give N-[(4-chloro-1-guanidino-7-isoquinolinyl)carbonyl]-DL-serine trifluoroacetate (68 mg, 0.19 mmol) as a white solid.

mp 203° C. (dec).

¹H (TFA-d, 400 MHz) δ 4.4 (1H, dd), 4.5 (1H, dd), 5.2-5.25 (1H, m), 8.35 (1H, s), 8.4 (1H, d), 8.5 (1H, d), 9.2 (1H, s) ppm.

LRMS 352 (MH⁺), 703 (M₂H⁺).

Anal. Found: C, 42.48; H, 3.69; N, 14.21. Calc for C₁₄H₁₄ClN₅O₄.CF₃CO₂H.0.4EtOAc: C, 42.19; H, 3.66; N, 13.98.

### Example 62

### (a) N-[(4-chloro-1-guanidino-7-isoquinolinyl)carbonyl]-DL-α-cyclopentylglycine t-butyl ester

### (b) N-[(4-Chloro-1-guanidino-7-isoquinolinyl)carbonyl]-DL-α-cyclopentylglycine trifluoroacetate

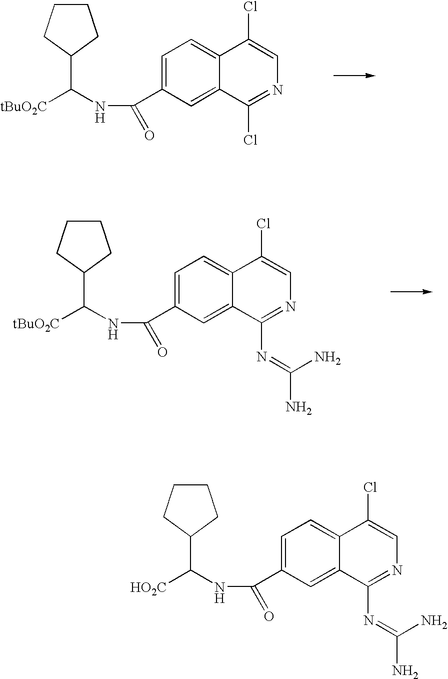

NaH (30 mg, 80% dispersion in mineral oil, 1.00 mmol) was added to a solution of guanidine hydrochloride (96 mg, 1.01 mmol) in DMSO (3 mL) and the solution was heated at 75-80° C. N-[(1,4-Dichloro-7-isoquinolinyl)carbonyl]-α-cyclopentylglycine t-butyl ester (170 mg, 0.40 mmol) was added and the mixture heated at 80° C. for 4.5 h. The cooled mixture was poured into water (25 mL) and extracted with EtOAc (3×20 mL). The combined organic extracts were washed with water, brine, dried (Na₂SO₄) and evaporated in vacuo to give N-[(4-chloro-1-guanidino-7-isoquinolinyl)carbonyl]-DL-α-cyclopentylglycine t-butyl ester (105 mg, 0.23 mmol) as a yellow solid.

An analytical sample was prepared as follows: this yellow solid was extracted with hot i-Pr₂O (3×20 mL), the hot solution was filtered, and on cooling gave the title compound as a pale yellow solid (40 mg) which was collected by filtration and dried in vacuo.

mp 219-221° C. (dec).

¹H (CDCl₃, 300 MHz) δ 1.4-1.8 (18H, m), 2.25-2.4 (1H, m), 4.7 (1H, dd), 6.2-6.9 (3H, br s), 6.95 (1H, d), 8.05 (1H, d), 8.1 (1H, s), 8.15 (1H, d), 9.15 (1H, s) ppm.

LRMS 446 (MH⁺), 891 (M₂H⁺).

Anal. Found: C, 58.83; H, 6.39; N, 15.34. Calc for C₂₂H₂₈ClN₅O₃.0.2H₂O: C, 58.78; H, 6.37; N, 15.30.

A solution of N-[(4-chloro-1-guanidino-7-isoquinolinyl)carbonyl]-DL-α-cyclopentylglycine t-butyl ester (65 mg, 0.15 mmol) in CF₃CO₂H (0.5 mL) was stirred at room temperature for 1 h. The solution was diluted with PhMe, evaporated in vacuo, and the residue was crystallised with EtOAc. This solid was then triturated with Et₂O to give N-[(4-chloro-1-guanidino-7-isoquinolinyl)carbonyl]-DL-α-cyclopentylglycine trifluoroacetate (52 mg, 0.10 mmol) as white powder.

mp 235° C. (dec).

¹H (TFA-d, 400 MHz) δ 1.4-1.8 (6H, m), 1.85-2.0 (2H, m), 2.4-2.55 (1H, m), 4.8 (1H, m), 8.25 (1H, d), 8.35 (1H, s), 8.45 (1H, d), 9.15 (1H, s) ppm.

LRMS 390 (MH⁺), 779 (M₂H⁺).

Anal. Found: C, 47.34; H, 4.36; N, 13.60. Calc for C₁₈H₂₀ClN₅O₃.CF₃CO₂H: C, 47.67; H, 4.20; N, 13.90.

### Example 63

### (a) N-Benzyl-N-[(4-chloro-1-guanidino-7-isoquinolinyl)carbonyl]glycine hydrochloride

### (b) N-Benzyl-N-[(4-chloro-1-guanidino-7-isoquinolinyl)carbonyl]glycine hydrochloride

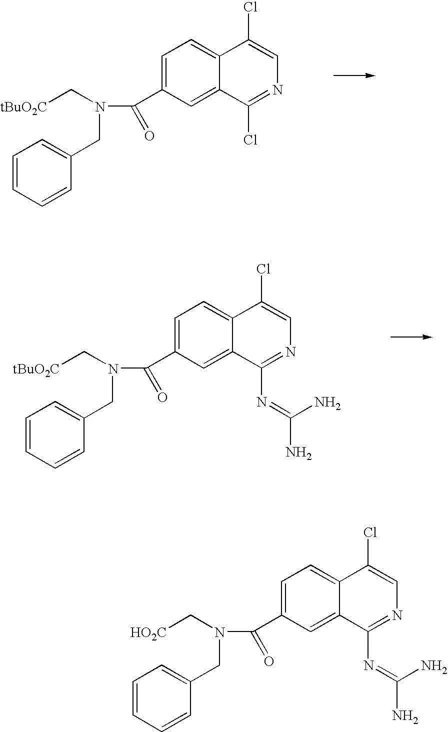

NaH (16 mg, 80% dispersion in mineral oil, 0.53 mmol) was added to a solution of guanidine hydrochloride (82 mg, 0.86 mmol) in DME (4 mL) and the mixture was heated at 60° C. for 30 min. A solution of N-benzyl-N-[(1,4-dichloro-7-isoquinolinyl)carbonyl]glycine t-butyl ester (95 mg, 0.21 mmol) in DME (2 mL) was added and the mixture was heated at 90° C. for 4 h. The cooled mixture was partioned between Et₂O and water, and the combined organic extracts were dried and evaporated in vacuo. The residue was dissolved in Et₂O and a solution of HCl in Et₂O (1 M) was added to give a precipitate of N-benyl-N-[(4-chloro-1-guanidino-7-isoquinolinyl)carbonyl]glycine hydrochloride. Evaporation of the ethereal mother liquors gave recovered, unreacted N-benzyl-N-[(1,4-dichloro-7-isoquinolinyl)carbonyl]glycine t-butyl ester which was again reacted with guanidine (as above) to give a second batch. Total yield: 70 mg, 0.15 mmol.

mp 130° C. (dec).

¹H (DMSO-d₆, 400 MHz) 5:6 mixture of rotamers, δ 1.2 (6/11 of 9H, s), 1.4 (5/11 of 9H, s), 4.0 (6/11 of 2H, s), 4.05 (5/11 of 2H, s), 4.5 (5/11 of 2H, s), 4.75 (6/11 of 2H, s), 7.2-7.5 (5H, m), 7.9-8.0 (1H, m), 8.2-8.3 (1H, m), 8.35 (1H, s), 8.75 (5/11 of 1H, s), 8.85 (6/11 of 1H, s) ppm.

LRMS 468 (MH⁺), 934 (M₂H⁺).

Anal. Found: C, 56.98; H, 5.71; N, 13.01. Calc for C₂₄H₂₆ClN₅O₃.HCl.0.5H₂O.0.2i-Pr₂O: C, 56.70; H, 5.82; N, 13.12.

A solution of N-benzyl-N-[(4-chloro-1-guanidino-7-isoquinolinyl)carbonyl]glycine hydrochloride (50 mg, 0.10 mmol) in CF₃CO₂H (1 mL) was stirred at room temperature for 1 h. The solution was diluted with PhMe, evaporated in vacuo, and the residue was triturated with Et₂O to afford a white solid (41 mg). This solid was dissolved in EtOAc and a solution of HCl in Et₂O was added which gave a precipitate. The mother liquors were decanted and the solid triturated with MeCN to give N-benyl-/N-[(4-chloro-1-guanidino-7-isoquinolinyl)carbonyl]glycine hydrochloride (16 mg, 0.04 mmol) as an off-white powder.

¹H (TFA-d, 400 MHz) 1:4 mixture of rotamers, δ 4.2 (1/5 of 2H, s), 4.45 (4/5 of 2H, s), 4.7 (4/5 of 2H, s), 4.95 (1/5 of 2H, s), 7.2 (2H, d), 7.3-7.4 (3H, m), 8.15 (1/5 of 1H, d), 8.2 (4/5 of 1H, d), 8.4 (1H, s), 8.45 (4/5 of 1H, d), 8.5 (1/5 of 1H, d), 8.7 (1/5 of 1H, s), 8.8 (4/5 of 1H, s) ppm.

LRMS 412 (MH⁺), 823 (M₂H⁺), 845 (M₂Na⁺).

Anal. Found: C, 52.55; H, 4.33; N, 15.10. Calc for C₂₀H₁₈ClN₅O₃.HCl.0.5H₂O: C, 52.52; H, 4.41; N, 15.32.

### Example 64

### (a) N-1(4-chloro-1-guanidino-7-isoquinolinyl)methyl]-N-methyl-DL-phenylglycine t-butyl ester

### (b) N-1(4-chloro-1-guanidino-7-isoquinolinyl)methyl]-N-methyl-DL-phenylglycine t-butyl ester dihydrochloride

### (c) N-1(4-Chloro-1-guanidino-7-isoquinolinyl)methyl]-N-methyl-DL-phenylglycine trifluoroacetate

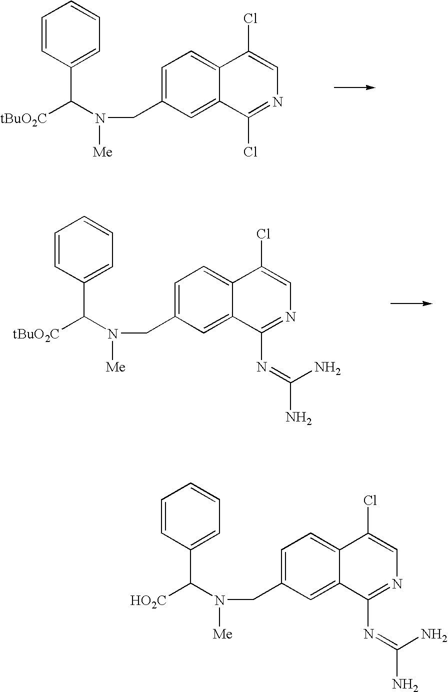

NaH (21 mg, 80% dispersion in mineral oil, 0.7 mmol) was added to t-BuOH (2.5 ml) and heated at 50° C. for 15 min. Guanidine hydrochloride (68 mg, 0.71 mmol) was added and heated at 50° C. for an additional 15 min. N-[(1,4-Dichloro-7-isoquinolinyl)methyl]-N-methyl-DL-phenylglycine t-butyl ester (102 mg, 0.24 mmol) was added and the mixture heated at 95° C. for 9.5 h. The cooled mixture was evaporated in vacuo and the residue was purified by column chromatography on silica gel using hexane-EtOAc (9:1), and then CH₂Cl₂-MeOH-0.880NH₃ (90:10:1) as eluant to give N-[(4-chloro-1-guanidino-7-isoquinolinyl)methyl]-N-methyl-DL-phenylglycine t-butyl ester (26 mg, 0.06 mmol) as a yellow gum. A portion of this material was dissolved in Et₂O, a solution of HCl in Et₂O was added and the resultant precipitate was triturated with hexane and then i-Pr₂O to give the corresponding dihydrochloride salt.

¹H (CD₃OD, 400 MHz) free base, δ 1.4 (9H, s), 2.2 (3H, s), 3.7 (1H, d), 3.8 (1H, d), 4.2 (1H, s), 7.3-7.4 (3H, m), 7.5 (2H, d), 7.9 (1H, d), 8.05 (1H, d), 8.05 (1H, s), 8.35 (1H, s) ppm.

LRMS 454 (MH⁺).

Anal. Found: C, 51.89; H, 6.01; N, 12.42. Calc for C₂₄H₂₈ClN₅O₂.2HCl.1.5H₂O: C, 52.04; H, 6.01; N, 12.64.

A solution of N-[(4-chloro-1-guanidino-7-isoquinolinyl)methyl]-N-methyl-DL-phenylglycine t-butyl ester (20 mg, 0.44 mmol) in CH₂Cl₂ (2 mL) was stirred with CF₃CO₂H (2 mL) at room temperature for 4 h. The solvents were evaporated in vacuo, and the residue was triturated with Et₂O and then EtOAc to give N-[(4-chloro-1-guanidino-7-isoquinolinyl)methyl]-N-methyl-DL-phenylglycine trifluoroacetate (6.5 mg, 0.02 mmol) as a white solid.

mp 180-182° C.

¹H (TFA-d, 400 MHz) 3:5 mixture of rotamers, δ 2.7 (5/8 of 3H, s), 3.05 (3/8 of 3H, s), 3.95-4.05 (3/8 of 1H, m), 4.55-4.7 (5/8 of 1H, m), 4.95-5.1 (1H, m), 5.35 (5/8 of 1H, s), 5.45 (3/8 of 1H, s), 7.4-7.7 (5H, m), 7.95 (3/8 of 1H, d), 8.1 (5/8 of 1H, d), 8.35 (1H, s), 8.4-8.65 (2H, m) ppm.

LRMS 400 (MH⁺).

Anal. Found: C, 50.10; H, 4.27; N, 12.90. Calc for C₂₀H₂₀ClN₅O₂.CF₃CO₂H.H₂O: C, 49.87; H, 4.37; N, 13.22.

### Example 65

### (a) N-benzyl-N-1(4-chloro-1-guanidino-7-isoquinolinyl)methyl]glycine t-butyl ester

### (b) N-Benzyl-N-1(4-chloro-1-guanidino-7-isoquinolinyl)methyl]glycine bistrifluoroacetate

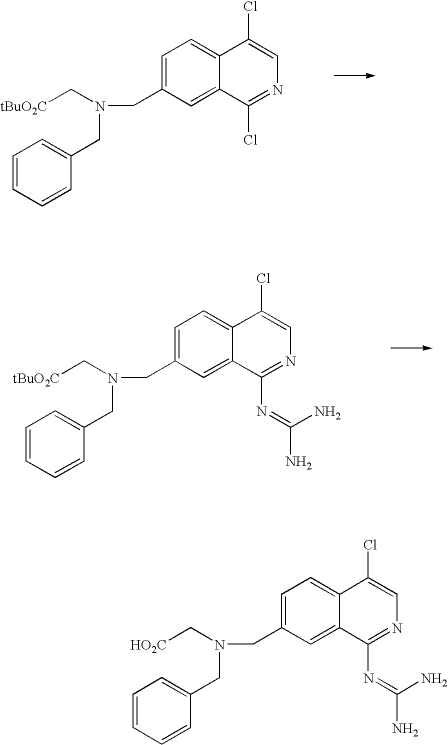

NaH (48.6 mg, 80% dispersion in mineral oil, 1.62 mmol) was added to t-BuOH (5 mL) and heated to 50° C. for 15 min. Guanidine hydrochloride (155 mg, 1.62 mmol) was added and heated at 50° C. for an additional 20 min. N-Benzyl-N-[(1,4-dichloro-7-isoquinolinyl)methyl]glycine t-butyl ester (40 mg, 0.09 mmol) added and the mixture was then heated at 95° C. for 20 h. The cooled mixture was evaporated in vacuo and the residue purified by column chromatography on silica gel using CH₂Cl₂-MeOH-0.880NH₃ (95:5:0.5), followed by trituration with hexane and crystallisation with i-Pr₂O, to give N-benzyl-N-[(4-chloro-1-guanidino-7-isoquinolinyl)methyl]glycine t-butyl ester (5 mg, 0.01 mmol) as a white solid.

¹H (CD₃OD, 400 MHz) δ 1.45 (9H, s), 3.15 (2H, s), 3.8 (2H, s), 3.95 (2H, s), 7.2-7.4 (5H, m), 7.85-7.95 (1H, m), 8.0-8.1 (2H, m), 8.5-8.55 (1H, m) ppm.

LRMS 454 (MH⁺), 907 (M₂H⁺).

Anal. Found: C, 62.57; H, 6.13; N, 15.17. Calc for C₂₄H₂₈ClN₅O₂.0.4H₂O: C, 62.51; H, 6.29; N, 15.19.

A solution of N-benzyl-N-[(4-chloro-1-guanidino-7-isoquinolinyl)methyl]glycine t-butyl ester (16 mg, 0.04 mmol) in CF₃CO₂H (1 mL) was stirred for at room temperature 1.5 h. The solution was diluted with PhMe, evaporated in vacuo, and the residue was triturated with Et₂O to give N-benzyl-N-[(4-chloro-1-guanidino-7-isoquinolinyl)methyl]glycine bistrifluoroacetate (6 mg, 0.02 mmol) as a white solid.

mp 199° C. dec.

¹H (TFA-d, 400 MHz) δ 4.2 (2H, s), 4.6 (1H, d), 4.75 (1H, d), 4.85 (1H, d), 4.95 (1H, d), 7.3-7.5 (5H, m), 8.0 (1H, d), 8.3 (1H, s), 8.45 (1H, d), 8.55 (1H, s) ppm.

LRMS 398 (MH⁺).

Anal. Found: C, 44.50; H, 3.81; N, 10.80. Calc for C₂₀H₂₀ClN₅O₂.2CF₃CO₂H.1.2H₂O: C, 44.52; H, 3.80; N, 10.82.

### Example 66

### (a) Nα-[(4-chloro-1-guanidino-7-isoquinolinyl)sulphonyl]-Nε-tert-butyloxycarbonyl-L-lysine tert-butyl ester

### (b) Nα-[(4-Chloro-1-guanidino-7-isoquinolinyl)sulphonyl]-L-lysine dihydrochloride

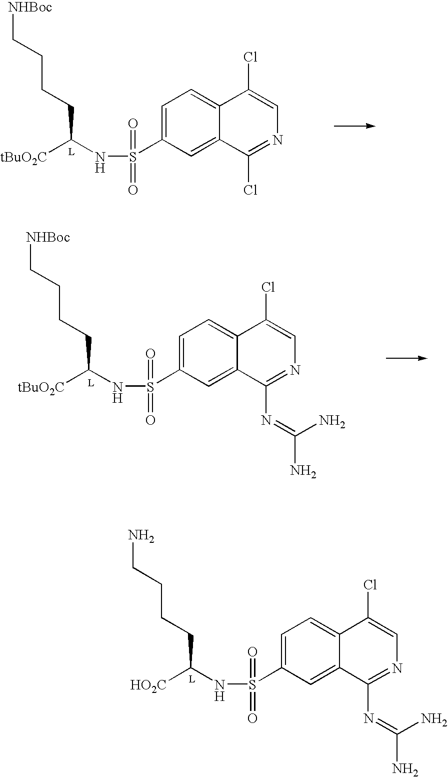

NaH (44 mg, 80% dispersion in mineral oil, 1.47 mmol) was added in a single portion to a solution of guanidine hydrochloride (224 mg, 2.35 mmol) in DMSO (5 ml) and stirred at room temperature until solution occurred. Nα-[(1,4-dichloro-7-isoquinolinyl)sulphonyl]-Nε-tert-butyloxycarbonyl-L-lysine-tert-butyl ester (330 mg, 0.59 mmol) was added and the solution stirred at 100° C. for 6 h. After cooling, the reaction mixture was quenched with water (30 ml), extracted with EtOAc (3×20 ml) and the combined organic extracts washed with water and brine. The organic solution was evaporated in vacuo and the residue purified by column chromatography upon silica gel using CH₂Cl₂-MeOH-0.880 NH₃ (90:10:1) as eluant to give Nα-[(4-chloro-1-guanidino-7-isoquinolinyl)sulphonyl]-Nε-tert-butyloxycarbonyl-L-lysine tert-butyl ester (152 mg, 0.26 mmol). An analytical sample was obtained by crystallisation from i-Pr₂O.

¹H (CDCl₃, 300 MHz) δ 1.15 (9H, s), 1.3-1.5 (13H, m), 1.5-1.8 (2H, m), 3.0-3.1 (2H, m), 3.8-3.9 (1H, m), 4.5-4.6 (1H, m), 5.2-5.4 (1H, m), 6.25-6.6 (3H, m), 8.0 (1H, d), 8.05 (1H, d), 8.1 (1H, s), 9.1 (1H, s) ppm.

LRMS 585 (MH⁺).

Anal. Found: C, 51.02; H, 6.32; N, 14.12. Calc for C₂₅H₃₇ClN₆O₆S: C, 51.32; H, 6.37; N, 14.36.

Nα-[(4-Chloro-1-guanidino-7-isoquinolinyl)sulphonyl]-Nε-tert-butyloxycarbonyl-L-lysine tert-butyl ester (119 mg, 0.20 mmol) was dissolved in EtOAc (10 nm) and saturated with gaseous HCl. After 20 min, the resultant white precipitate was obtained by filtration and recrystallised from EtOH to give Nα-[(4-chloro-1-guanidino-7-isoquinolinyl)sulphonyl]-L-lysine (13 mg, 0.03 mmol).

¹H (DMSO-d₆+CF₃CO₂D, 300 MHz) δ 1.1-1.7 (6H, m), 2.65-2.75 (2H, m), 3.75-3.80 (1H, m), 8.25 (1H, d), 8.35 (1H, d), 8.25 (1H, s), 8.9 (1H, s) ppm.

LRMS 429(MH⁺).

Anal. Found: C, 37.00; H, 4.93; N, 15.72. Calc for C₁₆H₂₁ClN₆O₄S.2HCl. H₂O.0.15 EtOH: C, 37.15; H, 4.95; N, 15.97.

### Example 67

### Nα-[(4-Chloro-1-guanidino-7-isoquinolinyl)sulphonyl]-D-lysine dihydrochloride

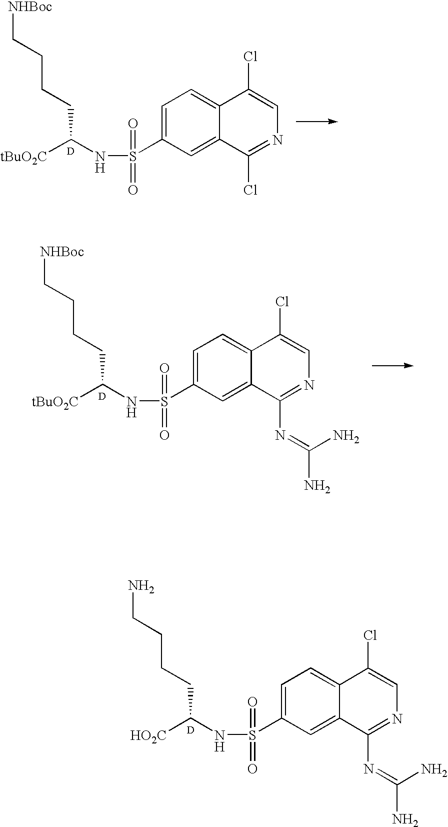

NaH (33 mg, 80% dispersion in mineral oil, 1.1 mmol) was added to a stirred solution of guanidine hydrochloride (170 mg, 1.78 mmol) in DMSO (3 ml) at 50° C. After 30 min, Nα-[(1,4-dichloro-7-isoquinolinyl)sulphonyl]-Nε-tert-butyloxycarbonyl-D-lysine tert-butyl ester (250 mg, 0.44 mmol) was added and the solution stirred at 90° C. for 8 h. The cooled mixture was poured into water and the precipitate extracted into Et₂O (4×15 ml). The combined organic extracts were washed with brine, dried (Na₂SO₄) and treated with 1N ethereal HCl. The solution was concentrated in vacuo, and the residue triturated with Et₂O and then EtOAc-EtOH to give Nα-[(4-chloro-1-guanidino-7-isoquinolinyl)sulphonyl]-D-lysine dihydrochloride (90 mg, 0.18 mmol).

¹H (DMSO-d₆, 400 MHz) δ 1.2-1.4 (2H, m), 1.4-1.7 (4H, m), 2.6-2.75 (2H, m), 3.9-4.0 (1H, m), 7.75-7.85 (3H, br s), 8.3 (1H, d), 8.35 (1H, d), 8.4 (1H, d), 8.4 (1H, s), 8.2-9.0 (3H, br m), 9.1 (1H, s) ppm.

LRMS 429 (MH⁺).

Anal. Found: C, 36.15; H, 5.10; N, 15.06. Calc for C₁₆H₂₁ClN₆O₄S. 2HCl.2H₂O.0.13 EtOAc: C, 36.18; H, 5.16; N, 15.25.

### Example 68

### (a) N-1(4-chloro-1-guanidino-7-isoquinolinyl)sulphonyl]-L-glutamine tert-butyl ester

(b) N-1(4-Chloro-1-guanidino-7-isoquinolinyl)sulphonyl]-L-glutamine trifluoroacetate

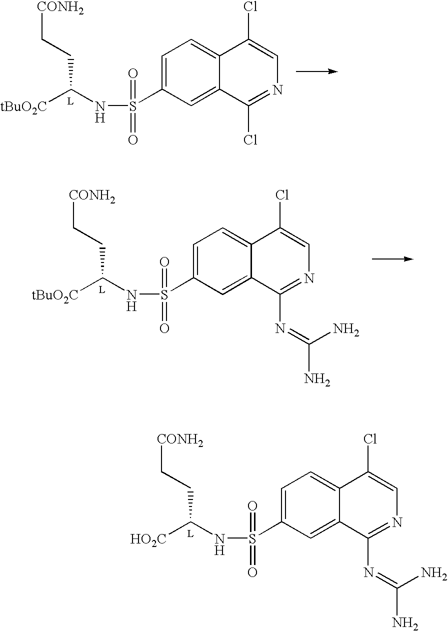

NaH (25 mg, 80% dispersion in mineral oil, 0.83 mmnol) was added to a solution of guanidine hydrochloride (128 mg, 1.34 mmol) in DMSO (2 ml) and stirred at 50° C. for 1 h. N-[(1,4-Dichloro-7-isoquinolinyl)sulphonyl]-L-glutamine tert-butyl ester (150 mg, 0.32 mmol) was added and the resultant solution stirred at 100° C. for 6 h, allowed to cool and then poured into water. The aqueous mixture was extracted with EtOAc (3×30 ml) and concentrated in vacuo. The residue was purified by column chromatography upon silica gel using CH₂Cl₂-MeOH-0.880 NH₃ (90:10:1) as eluant to give N-[(4-chloro-1-guanidino-7-isoquinolinyl)sulphonyl]-L-glutamine tert-butyl ester (30 mg, 0.06 mmol) as a buff-coloured powder.

¹H (DMSO-d₆, 300 MHz) δ 1.0-1.2 (9H, s), 1.6-1.75 (1H, m), 1.75-1.9 (1H, m), 2.05-2.15 (2H, m), 3.26-3.8 (1H, m), 6.65-6.75 (1H, br s), 7.0-7.45 (5H, br m), 7.95-8.1 (3H, m), 8.35 (1H, d), 9.0 (1H, s) ppm.

LRMS 485 (MH⁺).

added and the solution stirred at 90° C. for 8 h. The cooled mixture was poured into water and the precipitate extracted into Et₂O (4×15 ml). The combined organic extracts were washed with brine, dried (Na₂SO₄) and treated with 1N ethereal HCl. The solution was concentrated in vacuo, and the residue triturated with Et₂O and then EtOAc-EtOH to give Nα-[(4-chloro-1-guanidino-7-isoquinolinyl)sulphonyl]-D-lysine dihydrochloride (90 mg, 0.18 mmol).

¹H (DMSO-d₆, 400 MHz) δ 1.2-1.4 (2H, m), 1.4-1.7 (4H, m), 2.6-2.75 (2H, m), 3.9-4.0 (1H, m), 7.75-7.85 (3H, br s), 8.3 (1H, d), 8.35 (1H, d), 8.4 (1H, d), 8.4 (1H, s), 8.2-9.0 (3H, br m), 9.1 (1H, s) ppm.

LRMS 429 (MH⁺).

Anal. Found: C, 36.15; H, 5.10; N, 15.06. Calc for C₁₆H₂₁ClN₆O₄S. 2HCl.2H₂O.0.13 EtOAc: C, 36.18; H, 5.16; N, 15.25.

### Example 68

### (a) N-[(4chloro-1-guanidino-7-isoquinolinyl)sulphonyl]-L-glutamine tert-butyl ester

(b) N-[(4-Chloro-1-guanidino-7-isoquinolinyl)sulphonyl]-L-glutamine trifluoroacetate

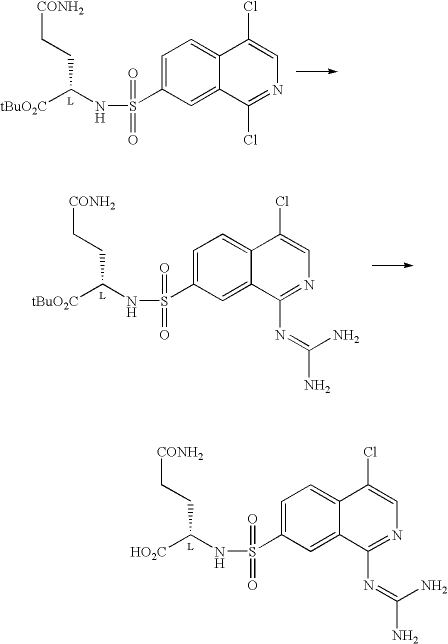

NaH (25 mg, 80% dispersion in mineral oil, 0.83 mmol) was added to a solution of guanidine hydrochloride (128 mg, 1.34 mmol) in DMSO (2 ml) and stirred at 50° C. for 1 h. N-[(1,4-Dichloro-7-isoquinolinyl)sulphonyl]-L-glutamine tert-butyl ester (150 mg, 0.32 mmol) was added and the resultant solution stirred at 100° C. for 6 h, allowed to cool and then poured into water. The aqueous mixture was extracted with EtOAc (3×30 ml) and concentrated in vacuo. The residue was purified by column chromatography upon silica gel using CH₂Cl₂-MeOH-0.880 NH₃ (90:10:1) as eluant to give N-[(4-chloro-1-guanidino-7-isoquinolinyl)sulphonyl]-L-glutamine tert-butyl ester (30 mg, 0.06 mmol) as a buff-coloured powder.

¹H (DMSO-d₆, 300 MHz) δ 1.0-1.2 (9H, s), 1.6-1.75 (1H, m), 1.75-1.9 (1H, m), 2.05-2.15 (2H, m), 3.26-3.8 (1H, m), 6.65-6.75 (1H, br s), 7.0-7.45 (5H, br m), 7.95-8.1 (3H, m), 8.35 (1H, d), 9.0 (1H, s) ppm.

LRMS 485 (MH⁺).

N-[(4-Chloro-1-guanidino-7-isoquinolinyl)sulphonyl]-L-glutamine tert-butyl ester (15 mg, 0.03 mmol) was dissolved in trifluoroacetic acid (1 ml) and the resultant solution stirred at room temperature for 1 h, diluted with toluene and concentrated to a residue. Trituration with Et₂O gave a powder to which was added MeOH and the suspension filtered. The filtrate was concentrated and then triturated with EtOAc to give N-[(4-chloro-1-guanidino-7-isoquinolinyl)sulphonyl]-L-glutamine trifluoroacetate (9 mg, 0.02 mmol).

¹H (DMSO-d₆+TFA-d, 300 MHz) δ 1.6-1.75 (1H, m), 1.8-2.0 (1H, m), 2.0-2.15 (2H, m), 3.8-3.9 (1H, m), 8.3 (1H, d), 8.35 (1H, d), 8.4 (1H, s), 8.8 (1H, s) ppm.

LRMS 429 (MH⁺).

### Example 69

### (2R)-1({4-Chloro-1-guanidino-7-isoquinolinyl}sulphonyl)-2-pyrrolidinecarboxamide

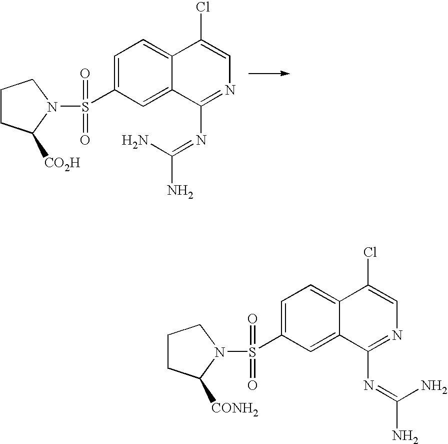

Oxalyl chloride (136 μl, 1.56 mmol) was added to a solution of N-[(4-chloro-1-guanidino-7-isoquinolinyl)sulphonyl]-D-proline (339 mg, 0.78 mmol) in CH₂Cl₂ (30 ml), followed by DMF (100 μl), and the reaction stirred at room temperature for 10 min. The mixture was evaporated in vacuo and azeotroped with toluene, to give an off-white solid. This was suspended in CH₂Cl₂ (15 ml), 0.880 NH₃ (760 μl, 7.8 mmol) added, and the reaction stirred at room temperature for 18 h. The mixture was partitioned between CH₂Cl₂ and water, and the layers separated. The aqueous phase was extracted with CH₂Cl₂, the combined organic solutions dried (MgSO₄) and evaporated in vacuo. The crude roduct was purified by column chromatography upon silica gel using an elution gradient of CH₂Cl₂-MeOH-0.880 NH₃ (100:0:0 to 95:5:0.1) to afford (2R)-t-({4-chloro-1-guanidino-7-isoquinolinyl}sulphonyl)-2-pyrrolidinecarboxamide (102 mg, 0.26 mmol) as a pale yellow solid.

¹H (d₄-MeOH, 400 MHz) δ 1.5-1.6 (1H, m), 1.7-2.0 (3H, m), 3.3-3.4 (1H, m), 3.55-3.65 (1H, m), 4.1-4.2 (1H, m), 8.1-8.2 (3H, m), 9.15 (1H, s) ppm.

LRMS 397 (MH⁺), 419 (MNa)⁺.

Anal. Found: C, 44.05; H, 4.42; N, 20.14. Calc for C₁₅H₁₇ClN₆O₃S+0.15 CH₂Cl₂: C, 44.43; H, 4.26; N, 20.52.

### Example 70

### (2R)-1-({4-Chloro-1-guanidino-7-isoquinolinyl}sulphonyl)-N-(2-hydroxyethyl)-2-pyrrolidinecarboxamide

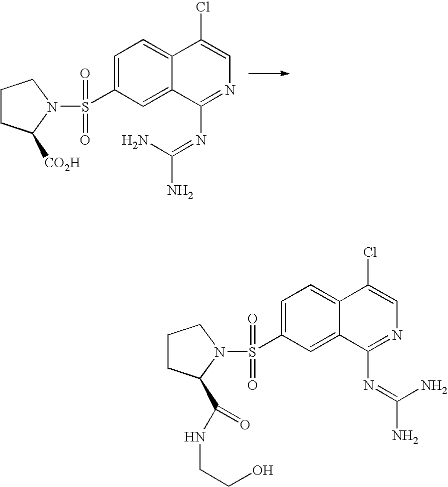

Oxalyl chloride (40 μl, 0.46 mmol) was added to a solution of N-[(4-chloro-1-guanidino-7-isoquinolinyl)sulphonyl]-D-proline (100 mg, 0.23 mmol) in CH₂Cl₂ (10 ml), followed by DMF (1 drop), and the reaction stirred at room temperature for 30 min. The mixture was evaporated in vacuo and azeotroped with toluene. The residue was dissolved in CH₂Cl₂ (5 ml), and added to a solution of ethanolamine (17 μl, 0.28 mmol) in CH₂Cl₂ (5 ml), the reaction stirred at room temperature for 2 h, then concentrated in vacuo. The crude product was purified by column chromatography upon silica gel using an elution gradient of CH₂Cl₂-MeOH-0.880 NH₃ (95:5:0.5 to 90:10:1) to afford (2R)-1-({4-chloro-1-guanidino-7-isoquinolinyl}sulphonyl)-N-(2-hydroxyethyl)-2-pyrrolidinecarboxamide (65 mg, 0.147 mmol) as a yellow foam.

¹H (DMSO-d₆, 300 MHz) δ 1.45-1.8 (4H, m), 3.15 (3H, m), 3.35-3.55 (3H, m), 4.1 (1H, m), 4.65 (1H, m), 7.9 (1H, m), 8.0 (1H, d), 8.15 (2H, m), 9.1 (1H, s) ppm.

LRMS 441, 443 (MH⁺)

Anal. Found: C, 43.96; H, 4.89; H, 17.47. Calc. for C₁₇H₂₁ClN₆O₄S.0.4CH₂Cl₂: C, 44.01; H, 4.63; N, 17.70%.

### Example 71

### (a) tert-butyl (2R)-1-({4-chloro-1-guanidino-7-isoquinolinyl}sulphonyl)-2-piperidinecarboxylate

(b) (2R)-1-({4-Chloro-1-guanidino-7-isoquinolinyl}sulphonyl)-2-piperidinecarboxylic acid hydrochloride

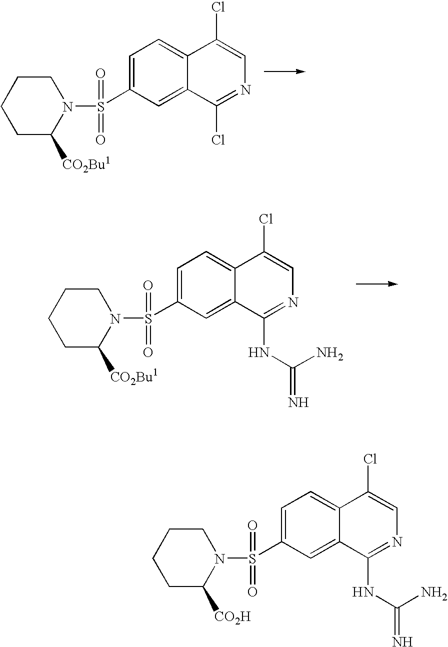

Guanidine hydrochloride (128 mg, 1.34 mmol) was added to a solution of NaH (32 mg, 80% dispersion in mineral oil, 1.07 mmol) in DME (5 ml), and the mixture stirred at 60° C., for 30 min. tert-Butyl (2R)-1-[(1,4-dichloro-7-isoquinolinyl)sulphonyl]-2-piperidinecarboxylate (150 mg, 0.34 mmol) was added and the reaction heated under reflux for 7 h, and stirred for a further 18 h at room temperature. The mixture was diluted with EtOAc, washed with water, brine, dried (MgSO₄), and evaporated in vacuo. The residual yellow gum was purified by column chromatography upon silica gel using CH₂Cl₂-MeOH-0.880 NH₃ (97:3:0.3) as eluant to give tert-butyl (2R)-1-({4-chloro-1-guanidino-7-isoquinolinyl)sulphonyl)-2-piperidinecarboxylate, as a yellow solid (126 mg, 0.27 mmol).

mp 157-158° C.

¹H (CDCl₃, 400 MHz) δ 1.3 (9H, s), 1.4 (1H, m), 1.6-1.8 (4H, m), 2.15 (1H, m), 3.3 (1, m), 3.85 (1H, m), 4.75 (1H, m), 8.0 (1H, d), 8.1 (1H, d), 8.15 (1H, s), 9.2 (1H, s) ppm.

LRMS 468 (MH⁺)

Anal. Found: C, 51.23; H, 5.68; N, 14.51. Calc. for C₂₀H₂₆ClN₅O₄S: C, 51.33; H, 5.60; N, 14.97%.

A solution of tert-butyl (2R)-1-({4-chloro-1-guanidino-7-isoquinolinyl}sulphonyl)-2-piperidinecarboxylate (50 mg, 0.107 mmol) in EtOAc saturated with HCl (10 ml), was stirred at room temperature for 2 h. The solution was concentrated in vacuo, and azeotroped several times with CH₂Cl₂ to give (2R)-1-({4-chloro-1-guanidino-7-isoquinolinyl}sulphonyl)-2-piperidinecarboxylic acid hydrochloride (37 mg, 0.083 mmol) as a white solid.

mp dec>220° C.

¹H (CD₃OD, 400 MHz) δ 1.35 (1H, m), 1.5 (1H, m), 1.65-1.8 (3H, m), 2.2 (1H, m), 3.2-3.3 (2H, m), 3.95 (1H, m), 8.3 (1H, d), 8.45 (2H, m), 8.9 (1H, s) ppm.

LRMS 412, 414 (MH⁺)

### Example 72

### (a) Methyl 4-[({4-chloro-1-guanidino-7-isoquinolinyl}sulphonyl)amino]-1-methyl-piperidinecarboxylate

### (b) 4-[({4-Chloro-1-guanidino-7-isoquinolinyl}sulphonyl)amino]-1-methyl-4-piperidinecarboxylic acid hydrochloride

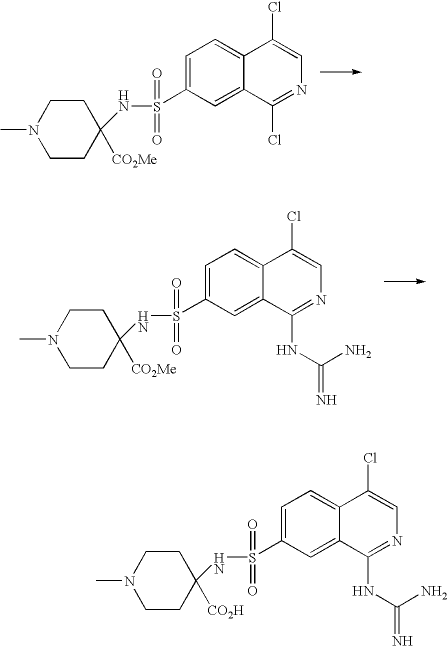

Guanidine hydrochloride (270 mg, 2.83 mmol) was added to a solution of NaH (65 mg, 80% dispersion in mineral oil, 2.16 mmol) in DMSO (6 ml), and the solution stirred at 60° C. for 30 min. Methyl 4-{[(1,4-dichloro-7-isoquinolinyl)sulphonyl]amino}-1-methyl-4-piperidinecarboxylate (300 mg, 0.7 mmol) was added and the reaction stirred at 80° C. for 5 h. Additional NaH (30 mg, 1 mmol), and guanidine hydrochloride (135 mg, 1.4 mmol) in DMSO (1 ml) were added, and the reaction heated for a further 2½ h. The cooled mixture was poured into water, and extracted with EtOAc. The combined organic extracts were washed with brine, dried (Na₂SO₄) and evaporated in vacuo. The residual yellow solid was purified by column chromatography upon silica gel using an elution gradient of CH₂Cl₂-MeOH-0.880 NH₃ (95:5:0.5 to 90:10:1) to afford methyl 4-[({4-chloro-1-guanidino-7-isoquinolinyl}sulphonyl)amino]-1-methyl-4-piperidinecarboxylate (232 mg, 0.51 mmol).

mp dec>205° C.

¹H (CD₃OD, 400 MHz) δ 2.05 (4H, m), 2.15 (3H, s), 2.25 (2H, m), 2.4 (2H, m), 3.4 (3H, s), 8.05-8.15 (3H, m), 9.1 (1H, s) ppm.

LRMS 455 (MH⁺)

Anal. Found: C, 47.17; H, 5.02; N, 17.96. Calc. for C₁₈H₂₃ClN₆O₄S.0.25H₂O: C, 47.06; H, 5.16; N, 18.29%.

A solution of methyl 4-[({4-chloro-1-guanidino-7-isoquinolinyl}sulphonyl)amino]-1-methyl-4-piperidinecarboxylate (100 mg, 0.22 mmol) in aqueous NaOH (2 ml, 2M, 4 mmol) and MeOH (5 ml) was stirred at 60° C. for 42 h. The cooled solution was neutralised using 2M HCl, and the mixture concentrated in vacuo, until precipitation occurred. The solid was filtered, dried and dissolved in concentrated HCl, and the solution evaporated in vacuo. The resulting solid was triturated with Et₂O, then i-PrOH, and dried under vacuum, to give 4-[({4-chloro-1-guanidino-7-isoquinolinyl}sulphonyl)amino]-1-methyl4-piperidinecarboxylic acid hydrochloride (18 mg, 0.035 mmol).

¹H (DMSO-d₆, 400 MHz) δ 2.1 (2H, m), 2.3 (2H, m), 2.7 (3H, s), 2.8-3.0 (2H, m), 3.3 (2H, m), 8.25-8.75 (7H, m), 9.1 (1H, s) ppm.

LRMS 441 (MH⁺)

### Example 73

### (a) tert-butyl N-[(1-guanidino-7-isoquinolinyl)sulphonyl]-D-prolinecarboxylate

### (b) N-[(1-Guanidino-7-isoquinolinyl)sulphonyl]-D-proline hydrochloride

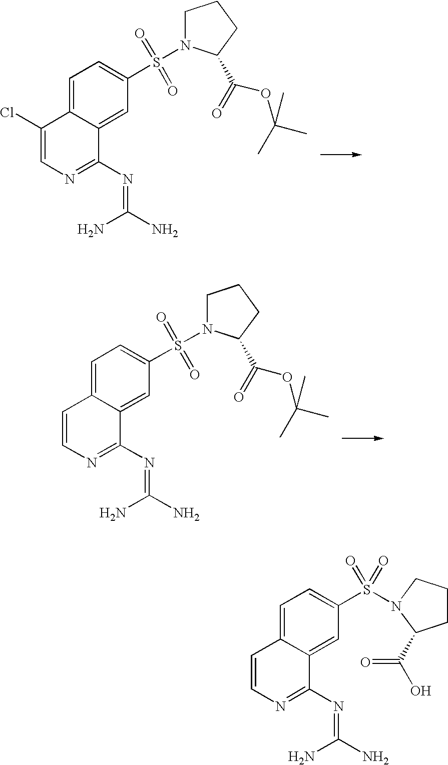

A mixture of tert-butyl N-[(4-chloro-1-guanidino-7-isoquinolinyl)sulphonyl]-D-prolinecarboxylate (200 mg, 0,44 mmol) and 5% palladium on charcoal (150 mg) in EtOH (30 ml) was hydrogenated at 50 psi and 50° C. for 24 h. The cooled mixture was filtered through Arbocel®, and the filter pad washed well with EtoH. The combined filtrates were concentrated in vacuo and the residue purified by column chromatography upon silica gel using an elution gradient of CH₂Cl₂-MeOH-0.880 NH₃ (97:3:0.3 to 95:5:0.5) to afford tert-butyl N-[(1-guanidino-7-isoquinolinyl)sulphonyl]-D-prolinecarboxylate (143 mg, 0.34 mmol) as an off-white solid.

¹H (CDCl₃, 400 MHz) δ 1.45 (9H, s), 1.75 (1H, m), 1.95 (3H, m), 3.4 (1H, m), 3.55 (1H, m), 4.3 (1H, m), 7.1 (1H, d), 7.75 (1H, d), 8.0 (1H, d), 8.15 (1H, d), 9.25 (1H, s) ppm.

LRMS 420(MH⁺)

A solution of tert-butyl N-[(1-guanidino-7-isoquinolinyl)sulphonyl]-D-prolinecarboxylate (130 mg, 0.31 mmol) in EtOAc saturated with HCl (7 ml) was stirred at room temperature for 1 h. The reaction mixture was evaporated in vacuo and azeotroped with CH₂Cl₂, to give N-[(1-guanidino-7-isoquinolinyl)sulphonyl]-D-proline hydrochloride (118 mg, 0.295 mmol) as a white solid.

mp dec>250° C.

¹H (DMSO-d₆, 400 MHz) δ 1.6 (1H, m), 1.75-1.95 (3H, m), 3.2 (1H, m), 3.4 (1H, m), 4.4 (1H, m), 7.7 (1H, m), 8.2 (2H, m), 8.3 (1H, m), 9.05 (1H, s) ppm.

LRMS 364 (MH⁺)

### Example 74

### 1-[({4-Chloro-1-guanidino-7-isoquinolinyl}sulphonyl)amino]-N-methyl-N-[2-(methylamino)ethyl]cyclopentanecarboxamide hydrochloride

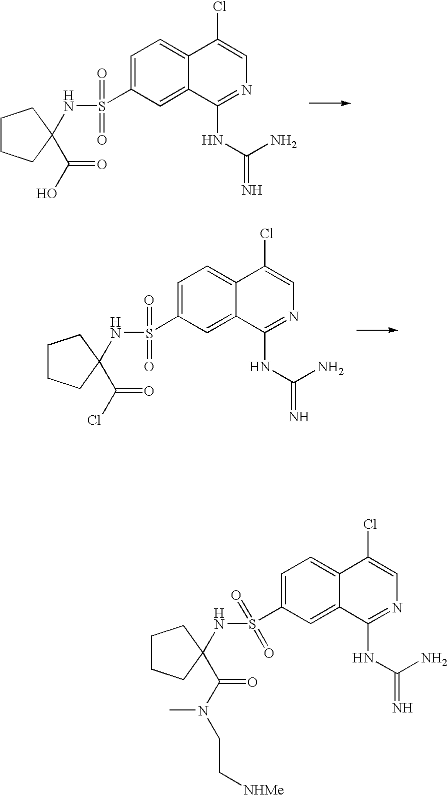

DMF (5 drops) was added to a suspension of 1-{([(1-guanidino-4-chloro-7-isoquinolinyl)sulphonyl]amino}cyclopentanecarboxylic acid hydrochloride (1.1 g, 2.46 mmol) in CH₂Cl₂ (100 ml), followed by oxalyl chloride (319 μl, 3.68 mmol), and the reaction stirred at room temperature for 45 min. Additional oxalyl chloride (106 μl, 1.23 mmol) was added, and stirring continued for a further 30 min. The mixture was evaporated in vacuo, triturated with CH₂Cl₂ and the residue then dissolved in CH₂Cl₂ (100 ml).

This solution of acid chloride (10 ml) was added to a solution of N,N′-dimethylethylenediamine (500 μl, 4.7 mmol) in CH₂Cl₂ (20 ml) and the resultant solution stirred at room temperature for 1 h. After evaporation to dryness, the residue was partitioned between water and CH₂Cl₂, the aqueous layer separated and extracted with EtOAc. The combined organic extracts were dried (Na₂SO₄), evaporated to a gum and purified by column chromatography upon silica gel eluting with CH₂Cl₂-MeOH-0.880 NH₃ (90:10:1) as eluant, to give an oil. This was dissolved in EtOAc, treated with ethereal HCl (1N), and the white precipitate, filtered and triturated with Et₂O, i-Pr₂O, and EtOH to yield the title compound (28 mg, 0.058 mmol).

mp 206° C. (foams).

¹H (DMSO-d₆, 400 MHz) δ 1.35 (4H, m), 1.7 (2H, m), 2.0 (2H, m), 2.6 (3H, s), 3.05 (2H, m), 3.2 (3H, s), 3.4 (2H, m), 3.5 (2H, m), 8.35 (1H, d), 8.4 (1H, d), 8.45 (1H, s), 8.6-8.8 (4H, m), 9.2 (1H, s) ppm.

LRMS 482, 484 (MH⁺).

### Example 75

### 1-[({4-Chloro1l-guanidino-7-isoquinolinyl}sulphonyl)amino]-N-(2-hydroxyethyl)-N-methylcyclopentanecarboxamide hydrochloride

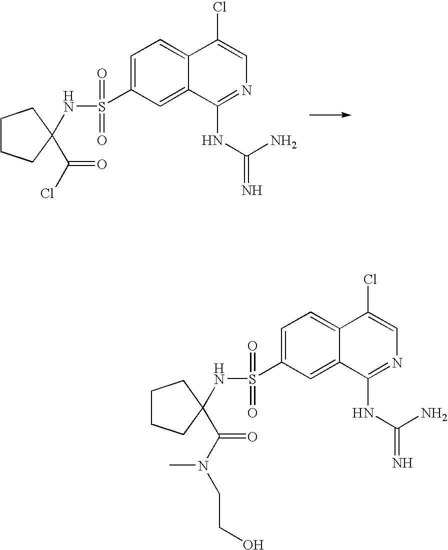

A suspension of 1-{[(4-chloro-1-guanidino-7-isoquinolinyl)sulphonyl]amino}cyclopentanecarbonyl chloride (110 mg, 0.245 mmol) in CH₂Cl₂ (10 ml) (prepared as described in example 76) was added over a minute to a solution of N-methylethanolamine (500 μl, 6.25 mmol) in CH₂Cl₂ (10 ml), and the resulting yellow solution stirred at room temperature for 72 h. The reaction mixture was evaporated in vacuo and the residue purified by column chromatography upon silica gel using CH₂Cl₂-MeOH-0.880 NH₃ (90:10:1) as eluant to give a clear gum. This was dissolved in EtOAc, ethereal HCl (1N) added, the mixture evaporated in vacuo and triturated with EtOAc. The resulting solid was filtered and dried under vacuum at 50° C. to give 1-[({4-chloro-1-guanidino-7-isoquinolinyl}sulphonyl)amino]-N-(2-hydroxyethyl)-N-methylcyclopentanecarboxamide hydrochloride.

¹H (DMSO-d₆, 400 MHz) δ 1.4 (4H, m), 1.8 (2H, m), 2.0 (2H, m), 2.6 (1H, m), 3.05-3.2 (4H, m), 3.35-3.6 (4H, m), 8.3 (1H, d), 8.4 (1H, d), 8.45 (1H, s), 8.55 (4H, m), 9.0 (1H, s), 11.0 (1H, s) ppm.

LRMS 468, 471 (MH⁺)

Anal. Found: C, 41.87; H, 5.55; N, 15.40. Calc. for C₁₉H₂₅ClN₆O₄S.HCl.2H₂O: C, 42.15; H, 5.58; N, 15.52%.

### Example 76

### 1-[({4-Chloro-1-guanidino-7-isoquinolinyl}sulphonyl)amino]-N-(2-methoxyethyl)cyclopentanecarboxamide hydrochloride

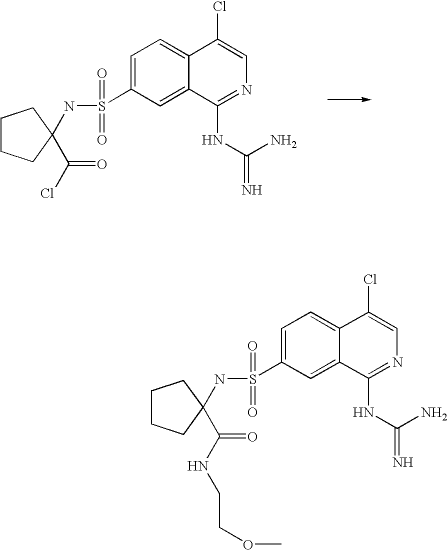

1-[({4-Chloro-1-guanidino-7-isoquinolinyl}sulphonyl)amino]-N-(2-methoxyethyl)cyclopentanecarboxamide was prepared from 2-methoxyethylamine and 1-([(4-chloro-1-guanidino-7-isoquinolinyl)sulphonyl]amino}cyclopentanecarbonyl chloride, following the same procedure described in example 76. This product was treated with ethereal HCl (1N) and the mixture evaporated in vacuo. The residual solid was dissolved in EtOH, water (1 drop) added, the solution concentrated in vacuo until precipitation occured, and the resulting solid filtered, washed with Et₂O, and dried under vacuum, at 50° C., to afford 1-[((4-chloro-1-guanidino-7-isoquinolinyl}sulphonyl)amino]-N-(2-methoxyethyl)cyclopentanecarboxamide hydrochloride (35 mg, 28%).

¹H (DMSO-d₆, 300 MHz) δ 1.3-1.5 (4H, m), 1.9 (4H, m), 2.95 (2H, m), 3.2 (5H, m), 7.55 (1H, t), 8.2 (1H, s), 8.35 (2H, m), 8.45 (1H, s), 8.6 (4H, m), 9.1 (1H, s) ppm.

LRMS 469, 471 (MH⁺)

Anal. Found: C, 43.33; H, 5.38; N, 15.82. Calc. for C₁₉H₂₅ClN₆O₄S.HCl.1.2H₂O: C, 43.30; H, 5.43; N, 15.95%.

### Example 77

### (a) N-(2-tert-butyl aminoethylcarbamate)-1-[({4-chloro-1-guanidino-7-isoquinolinyl}sulphonyl)amino]cyclopentanecarboxamide

### (b) N-(2-Aminoethyl)-1-[({4-chloro-1-guanidino-7-isoquinolinyl}sulphonyl)amino]cyclopentane-carboxamide dihydrochloride

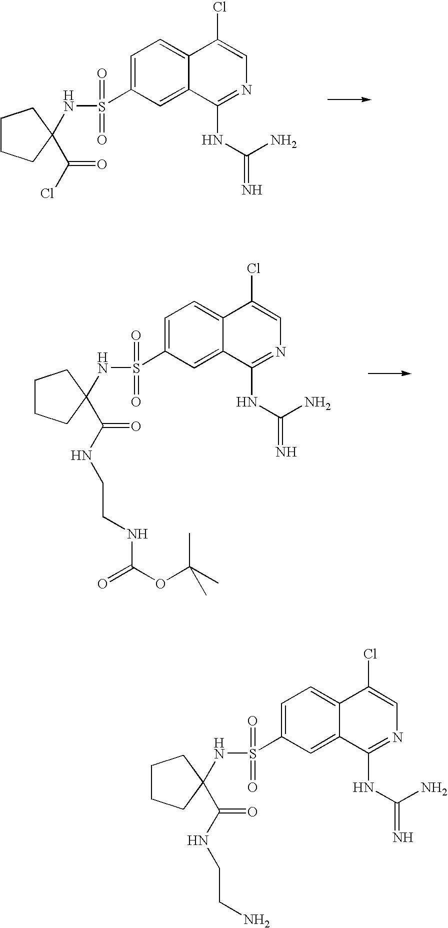

A suspension of 1-{[(4-chloro-1-guanidino-7-isoquinolinyl)sulphonyl]amino}cyclopentanecarbonyl chloride (220 mg, 0.49 mmol) was added to a solution of tert-butoxy 2-aminoethylcarbamate (250 mg, 1.56 mmol) in CH₂Cl₂ (10 ml), and the reaction stirred at room temperature for 18 h. The mixture was evaporated in vacuo and the residue purified by column chromatography upon silica gel using CH₂Cl₂-MeOH-0.880 NH₃(90:10:1) as eluant to give a yellow oil. This product was crystallised from MeOH-i-Pr₂O to afford N-(2-tert-butyl aminoethylcarbamate)-1-[((4-chloro-1-guanidino-7-isoquinolinyl}sulphonyl)amino]cyclopentanecarboxamide (27 mg, 0.05 mmol) as a pale yellow solid.

¹H (CDCl₃, 300 MHz) δ 1.3 (11H, m), 1.4 (2H, m), 1.8 (2H, m), 1.9 (2H, m), 2.45 (2H, m), 3.05 (4H, m), 5.65 (1H, m), 6.8 (4H, m), 7.1 (1H, m), 7.2 (1H, m), 7.9 (3H, m), 9.1 (1H, s) ppm.

LRMS 576 (MNa⁺)

A solution of N-(2-tert-butyl aminoethylcarbamate)-1-[({4-chloro-1-guanidino-7-isoquinolinyl}sulphonyl)amino]cyclopentanecarboxamide (20 mg, 0.036 mmol) in ethereal HCl (1 ml, 1N) was stirred at room temperature for 2 h. The reaction mixture was diluted with MeOH, concentrated in vacuo, and the residue triturated with Et₂O, then i-Pr₂O, and dried, to give N-(2-aminoethyl)-1-[({4-chloro-1-guanidino-7-isoquinolinyl}sulphonyl)amino]cyclopentanecarboxamide dihydrochloride (16 mg, 0.30 mmol) as an off-white powder

¹H (DMSO-d₆, 400 MHz) δ 1.6 (4H, m), 1.85 (2H, m), 1.9 (2H, m), 2.8 (2H, m), 3.2 (2H, m), 5.4 (1H, br s), 7.9 (2H, br s), 8.05 (1H, m), 8.2 (1H, s), 8.4 (1H, m), 8.45 (1H, s), 8.55-8.75 (4H, M), 9.25 (1H, s) ppm.

LRMS 454 (MH⁺)

### Example 78

### 4-Chloro-1-guanidino-N-[1-(morpholinocarbonyl)cyclopentyl]-7-isoquinolinesulphonamide hydrochloride

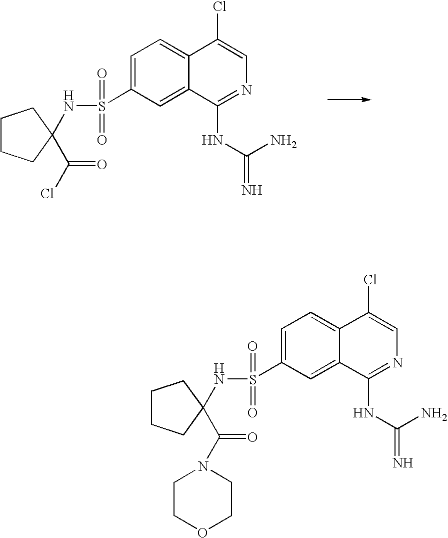

The title compound was prepared from 1-{[(4-chloro-1-guanidino-7-isoquinolinyl)sulphonyl]amino}cyclopentanecarbonyl chloride, and morpholine, following a similar procedure to that described in example 74.

¹H (DMSO-d₆, 300 MHz) δ 1.35 (4H, m), 1.7 (2H, m), 2.0 (2H, m), 3.4-3.65 (8H, m), 8.35-8.65 (8H, m), 8.95 (1H, s) ppm.

LRMS 480, 482 (MH⁺)

### Example 79

### 4-Chloro-1-guanidino-N-{1-[(4-methylpiperazino)carbonyl]cyclopentyl}-7-isoquinolinesulphonamide dihydrochloride

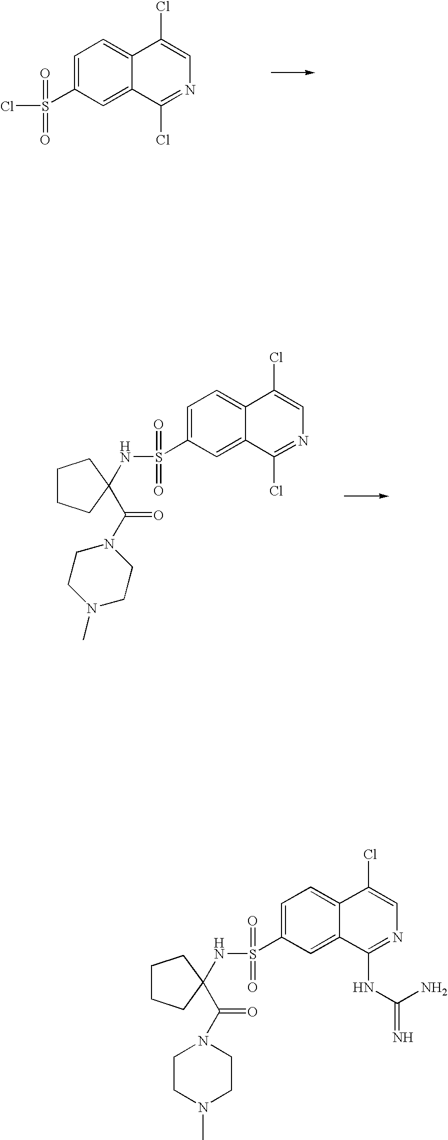

Triethylamine (1.36 ml, 10.0 mmol) was added to a solution of (1-aminocyclopentyl(4-methyl-1-piperazinyl)methanone dihydrochloride (567 mg, 2.0 mmol) and 1,4-dichloro-7-isoquinolinesulphonyl chloride (592 mg, 2.0 mmol) in CH₂Cl₂ (25 ml), and the reaction stirred at room temperature for 18 h. The mixture was concentrated in vacuo and the residue partitioned between EtOAc and water, and the layers separated. The organic phase was washed with water, extracted with HCl (2N), and these combined acidic extracts washed with EtOAc, and re-basified using Na₂CO₃. This aqueous solution was extracted with EtOAc, the combined organic extracts washed with brine, dried (Na₂SO₄) and evaporated in vacuo to give a foam. This was crystallised from CH₂Cl₂-i-Pr₂O to afford 1,4-dichloro-N-{1-[(4-methyl-1-piperazinyl)carbonyl]cyclopentyl}-7-isoquinolinesulphonamide (153 mg, 0.33 mmol) as a solid.

¹H (CDCl₃, 300 MHz) δ 1.5-1.75 (6H, m), 2.25-2.45 (9H, m), 3.6 (4H, m), 5.1 (1H, s), 8.25 (1H, d), 8.35 (1H, d), 8.5 (1H, s), 8.9 (1H, s) ppm.

Anal. Found: C, 49.12; H, 5.02; N, 1.06. Calc. for C₂₀H₂₄Cl₂N₄O₃S.0.3CH₂Cl₂: C, 49.07; H, 4.99; N, 11.28%.

NaH (22 mg, 80% dispersion in mineral oil, 0.73 mmol) was added to a solution of guanidine hydrochloride (142 mg, 1.49 mmol) in DMSO (2 ml), and the solution stirred at 50° C. for 30 min. 1,4-Dichloro-N-{1-[(4-methyl-1-piperazinyl)carbonyl]cyclopentyl}-7-isoquinolinesulphonamide (140 mg, 0.28 mmol) was added and the reaction stirred at 90° C. for 5 h. The cooled reaction was poured into water, the mixture extracted with EtOAc, and the combined extracts washed with brine, dried (Na₂SO₄) and evaporated in vacuo. The residual yellow foam was dissolved in i-PrOH, ethereal HCl (1N) was added, the solution evaporated in vacuo and the product suspended in ethanol. This mixture was filtered, the filtrate cooled in an ice-bath,.and the resulting solid filtered, washed with EtOH, and dried, to give 4-chloro-1-guanidino-N-{1-[(4-methyl-1-piperazinyl)carbonyl]cyclopentyl}-7-isoquinolinesulphonamide dihydrochloride (68 mg, 0.12 mmol).

¹H (DMSO-d₆, 300 MHz) δ 1.35 (4H, m), 1.7 (2H, m), 2.0 (2H, m), 2.75 (3H, s), 3.0 (2H, m), 3.25-3.45 (4H, m), 4.4 (2H, m), 8.3 (1H, d), 8.4 (1H, d), 8.45 (1H, s), 8.6 (4H, m), 8.7 (1H, s), 9.1 (1H, s), 11.15 (2H, br s) ppm.

LRMS 494, 496 (MH⁺)

### Example 80

### (a) N-[(4-chloro-1-guanidino-7-isoquinolinyl)sulphonyl]-N-(methyl)cycloleucine ethyl ester

### (b) N-1(4-Chloro-1-guanidino-7-isoquinolinyl)sulphonyl]-N-(methyl)cycloleucine hydrochloride

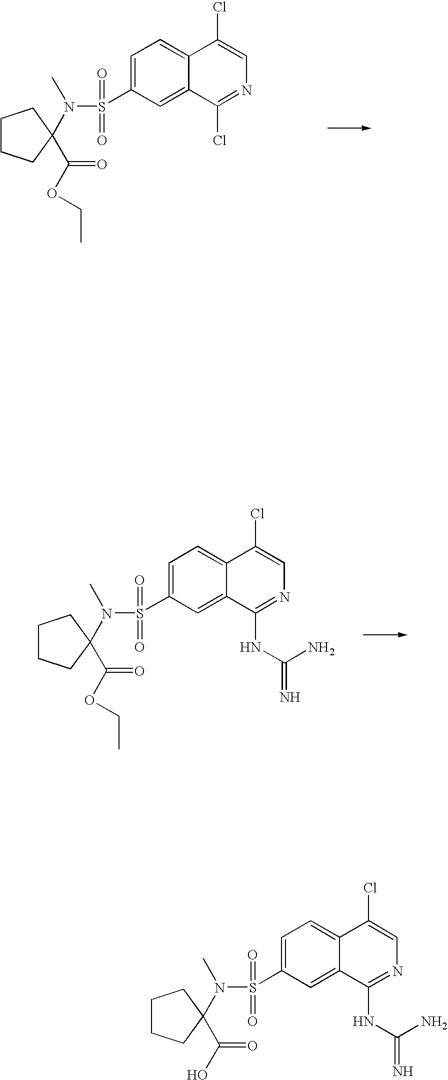

NaH (31 mg, 80% dispersion in mineral oil, 1.04 mmol) was added to a solution of guanidine hydrochloride (164 mg, 1.67 mmol) in DMSO (4 ml), and the solution heated at 50° C. for 1 h. N-[(1,4-Dichloro-7-isoquinolinyl)sulphonyl]-N-(methyl)cycloleucine ethyl ester (180 mg, 0.42 mmol) in DMSO (2 ml) was added, and the reaction heated at 80° C. for 3 h. The cooled reaction mixture was poured into water, and extracted with EtOAc. The combined organic extracts were washed with brine, dried (MgSO₄), and evaporated in vacuo. The residual yellow oil was purified by column chromatography upon silica gel using CH₂Cl₂-MeOH-0.880 NH₃ (90:10:1) as eluant, and recrystallised from EtOAc to afford N-[(4-chloro-1-guanidino-7-isoquinolinyl)sulphonyl]-N-(methyl)cycloleucine ethyl ester (105 mg, 0.23 mmol) as a yellow solid.

mp 186-188° C.

¹H (DMSO-d₆, 400 MHz) δ 1.1 (3H, t), 1.55 (4H, m), 2.0 (2H, m), 2.2 (2H, m), 2.95 (3H, s), 4.0 (2H, q), 7.2-7.4 (4H, br s), 8.05 (2H, m), 8.15 (1H, s), 9.1 (1H, s) ppm.

LRMS 454, 456 (MH⁺)

Anal. Found: C, 50.04; H, 5.38; N, 15.31. Calc. for C₁₉H₂₄ClN₅O₄S.0.2H₂O: C, 49.88 H, 5.38; N, 15.31%.

A solution of N-[(4-chloro-1-guanidino-7-isoquinolinyl)sulphonyl]-N-(methyl)cycloleucine ethyl ester (80 mg, 0.176 mmol) in NaOH (1 ml, 2N) and MeOH (10 ml) was stirred at 70° C. for 18 h. The cooled mixture was neutralised using HCl (2N), and the MeOH was removed in vacuo. The resulting precipitate was filtered off, washed with water and re-dissolved in concentrated HCl. This solution was evaporated in vacuo, azeotroped with toluene, the residue dissolved in EtOH and filtered. The filtrate was evaporated in vacuo and the resulting solid recrystallised from i-PrOH, to give N-[(4-chloro-1-guanidino-7-isoquinolinyl)sulphonyl]-N-(methyl)cycloleucine hydrochloride (18 mg, 0.039 mmol) as a yellow solid.

mp 225° C. (dec.).

¹H (DMSO-d₆+TFA-d, 400 MHz) δ 1.4-1.6 (4H, m), 1.95-2.0 (2H, m), 2.15-2.25 (2H, m), 3.0 (3H, s), 8.3 (1H, d), 8.35 (11H, d), 8.45 (1H, s), 8.95 (1H, s) ppm.

LRMS 426, 428 (MH⁺).

Anal. Found: C, 41.50; H, 4.79; N, 13.82. Calc for C₁₇H₂₀ClN₅O₄S.HCl.1.8H₂O: C, 41.27; H. 5.01; N, 14.15.

### Example 81

### (a) N-[(4-Bromo-1-guanidino-7-isoquinolinyl)sulphonyl]-D-proline tert-butyl ester hydrochloride

### (b) N-[(4-Bromo-1-guanidino-7-isoquinolinyl)sulphonyl]-D-proline hydrochloride

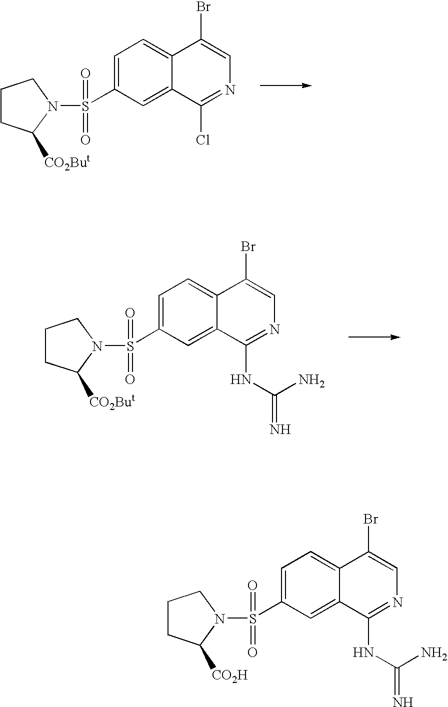

NaH (48 mg, 80% disperson in mineral oil, 1.6 mmol) was added to a solution of guanidine hydrochloride (233 mg, 2.43 mmol) in DMSO (8 ml) and the solution stirred at room temperature for 30 min. N-[(4-Bromo-1-chloro-7-isoquinolinyl)sulphonyl]-D-proline tert-butyl ester (290 mg, 0.61 mmol), was added and the reaction stirred at 60° C. for 2 h, and allowed to cool to room temperature overnight. The mixture was poured into water, and extracted with EtOAc. The combined organic extracts were washed with brine, dried (MgSO₄) and evaporated in vacuo. The residual yellow oil was purified by column chromatography upon silica gel using CH₂Cl₂-MeOH-0.880 NH₃ (97.5:2.5:0.25) as eluant, to give a yellow foam. This was dissolved in Et₂O, treated with ethereal HCl, the mixture evaporated in vacuo and the residue triturated with Et₂O to give N-[(4-bromo-1-guanidino-7-isoquinolinyl)sulphonyl]-D-proline tert-butyl ester hydrochloride (166 mg, 0.31 mmol) as a white solid.

mp. 203° C.

¹H (DMSO-d₆, 300 MHz) δ 1.4 (9H, s), 1.65 (1H, m), 1.8 (2H, m), 2.0 (1H, m), 3.35 (1H, m), 3.45 (1H, m), 8.35 (2H, m), 8.5-8.8 (5H, m), 9.1 (1H, s), 11.4 (1H, s) ppm.

LRMS 497, 499 (MH⁺)

Anal. Found: C, 41.96: H, 4.65; N, 12.65. Calc. for C₁₉H₂₄BrN₅O₄S.HCl.0.5H₂O: C, 41.96:, 4.82; N, 12.88%.

N-[(4-Bromo-1-guanidino-7-isoquinolinyl)sulphonyl]-D-proline tert-butyl ester hydrochloride (150 mg, 0.28 mmol) was treated with an ice-cold solution of HCl in EtOAc (20 ml), and the reaction allowed to warn to room temperature, and stirred for 4 h. The solution was concentrated in vacuo and the crude product purified by column chromatography upon silica gel using CH₂Cl₂-MeOH-0.880 NH₃ (90:10:1) as eluant. The product was treated with ethereal HCl, the resulting precipitate filtered, washed with Et₂O and dried to afford N-[(4-bromo-1-guanidino-7-isoquinolinyl)sulphonyl]-D-proline hydrochloride (75 mg, 0.156 mmol) as a white powder.

¹H (DMSO-d₆, 300 MHz) δ 1.6 (1H, m), 1.7-2.0 (3H, m), 3.2-3.45 (2H, m), 4.4 (1H, m), 8.3 (2H, m), 8.5-8.85 (5H, m), 9.15 (1H, s) ppm.

LRMS 443 (MH⁺)

Anal. Found: C, 35.56; H, 3.54; N, 13.52. Calc. for C₁₅H₁₆BrN₅O₄S.HCl.1.5H₂O: C, 35.62: H, 3.99; N, 13.85%.

### Example 82

### (2R)-1-({4-Chloro-1-guanidino-7-isoquinolinyl}sulphonyl)-N-[2dimethylamino)ethyl]-2-pyrrolidinecarboxamide

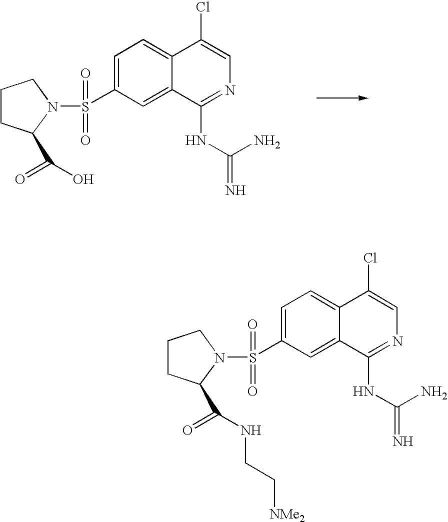

N-[(4-Chloro-1-guanidino-7-isoquinolinyl)sulphonyl]-L-proline hydrochloride (300 mg, 0.69 mmol) was suspended in a solution of DMF (5 drops) and CH₂Cl₂ (15 ml), and oxalyl chloride (150 μl, 1.72 mmol) added dropwise. The reaction was stirred at room temperature for 3 h, then concentrated in vacuo and azeotroped with toluene. The residue was dissolved in CH₂Cl₂ (15 ml), N-(2-aminoethyl)-N,N-dimethylamine (1 ml, 0.9 mmol) added and the reaction stirred at room temperature for 2 h. The mixture was evaporated in vacuo, the residue partitioned between EtOAc and Na₂CO₃ solution, the layers separated, and the organic phase washed with brine, dried (Na₂SO₄) and evaporated in vacuo. The residual yellow solid was purified by column chromatography upon silica gel using an elution gradient of CH₂Cl₂-MeOH-0.880 NH₃ (95:5:0.5 to 90:10:1) to give (2R)-1-({4-chloro-1-guanidino-7-isoquinolinyl}sulphonyl)-N-[2-(dimethylamino)ethyl]-2-pyrrolidinecarboxamide (195 mg, 0.42 mmol) as a yellow solid.

¹H (DMSO-d₆, 400 MHz) δ 1.55 (1H, m), 1.65 (1H, m), 1.7 (2H, m), 2.15 (6H, s), 2.25 (2H, t), 3.2 (3H, m), 3.5 (1H, m), 4.1 (1H, dd), 7.2-7.4 (4H, br s), 7.8 (1H, m), 8.0 (1H, d), 8.15 (2H, m), 9.1 (1H, s) ppm.

Anal. Found: C, 47.67; H, 5.61; N, 20.31. Calc. for C₁₉H₂₆ClN₇O₃S.0.5H₂O: C, 47.84; H, 5.71; N, 20.56%.

### Example 83

### 1-{({4-Chloro-1-guanidino-7-isoquinolinyl}sulphonyl)[2-(dimethylamino)ethyl}amino]-N-(2-hydroxyethyl)-N-methylcyclopentanecarboxamide dihydrochloride

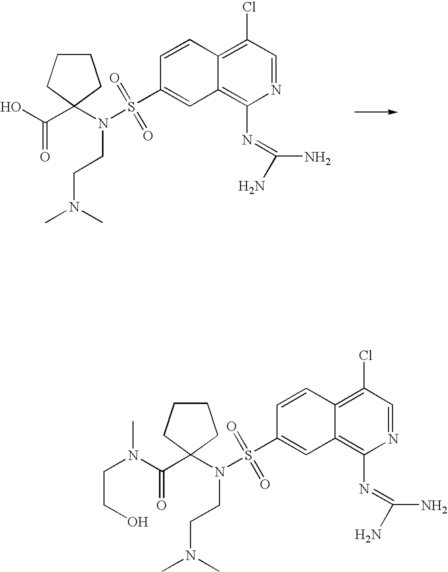

N-[(4-chloro-1-guanidino-7-isoquinolinyl)sulphonyl]-N-[2-(dimethylamino)ethyl]cycloleucine dihydrochloride (170 mg, 0.31 mmol) was dissolved in DMF (10 μl) and CH₂Cl₂ (15 ml). Oxalyl chloride (100 μl, 1.15 mmol) was added and the mixture stirred at room temperature for 3 h. The solvent was removed in vacuo, replaced with fresh CH₂Cl₂, N-methylethanolamine (230 μl, 2.86 mmol) in CH₂Cl₂ (10 ml) added, and the reaction stirred for 2 h. The solvent was removed in vacuo and the resultant gum extracted with Et₂O and EtOAc. These combined organic extracts were concentrated in vacuo, and the crude product purified by column chromatography upon silica gel eluting with CH₂Cl₂-MeOH-0.880 NH₃ (90:10:1). The resulting yellow oil was dissolved in EtOAc, and acidified with ethereal HCl (1N) to give the title compound as a cream solid (17 mg, 0.03 mmol).

¹H (DMSO-d₆+TFA-d, 300 MHz) δ 1.55 (4H, m), 2.0 (2H, m), 2.4 (2H, m), 2.6 (3H, s), 2.9 (6H, s), 3.35 (2H, m), 3.5 (3H, m), 3.95 (2H, m), 4.3 (2H, t), 8.4 (3H, m), 8.5 (1H, s), 9.35 (1H, s) ppm.

LRMS 540, 542 (MH⁺).

### Example 84

### (a) Ethyl N-[(4-bromo-1-guanidino-7-isoquinolinyl)sulphonyl]-N-[2-(dimethylamino)ethyl]-cycloleucine dihydrochloride

### (b) N{4-Bromo-1-guanidino-7-isoquinolinyl}sulphonyl)-N-[2dimethylamino)ethyl]cycloleucine dihydrochloride

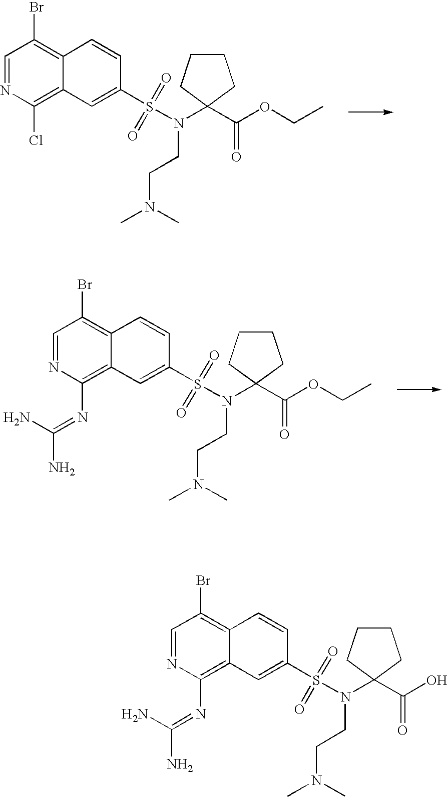

A mixture of NaH (28 mg, 80% in mineral oil, 0.93 mmol) and guanidine hydrochloride (126 mg, 1.32 mmol) in dry DMSO (3 ml) was heated at 50° C. for 30 min. N-[(4-Bromo-1-chloro-7-isoquinolinyl)sulphonyl]-N-[2-(dimethylamino)ethyl]cycloleucine hydrochloride (150 mg, 0.26 mmol) was added and the mixture heated to 90° C. for 1 h, cooled, poured into water and extracted with EtOAc (3×). The combined organic extracts were washed with water and brine, dried (Na₂SO₄) and concentrated in vacuo to a yellow gum. After column chromatography on silica gel eluting with CH₂Cl₂-MeOH-0.880 NH₃ (95:5:0.5), the residue was dissolved in EtOAc and acidified with ethereal HCl (1N) to afford a white precipitate. This was filtered, dried and recrystallised from EtOH to give a white solid (20 mg, 0.04 mmol). Concentration of the mother liquors afforded a second crop (95 mg, 0.17 mmol) of ethyl N-[(4-bromo-1-guanidino-7-isoquinolinyl)sulphonyl]-N-[2-(dimethylamino)ethyl]cycloleucine dihydrochloride.

¹H (DMSO-d₆, 300 MHz) δ 1.15 (3H, t), 1.6 (4H, m), 2.0 (2H, m), 2.3 (2H, m), 2.9 (6H, s), 3.5 (2H, m), 3.95 (2H, m), 4.0 (2H, q), 8.34 (2H, s), 8.6 (1H, s), 9.4 (1H, s), 11.6 (1H, br s) ppm.

LRMS 555, 557 (MH⁺).

Anal. Found: C, 39.67; H, 5.61; N, 12.51. Calc. for C₂₂H₃₁BrN₆O₄S.2HCl.2H₂O: C, 39.77; H, 5.61; N, 12.65%.

Ethyl N-[(4-Bromo-1-guanidino-7-isoquinolinyl)sulphonyl]-N-[2-(dimethylamino)ethyl]cycloleucine dihydrochloride (95 mg, 0.17 mmol) in EtOH (3 ml) was treated with NaOH (4N, 8 ml) and the solution stirred at 60° C. for 5 h and allowed to stand for 60 h at room temperature. The reaction mixture was acidified using 2N HCl, concentrated in vacuo and the residue azeotroped with i-PrOH to give an off-white solid. This was extracted into MeOH, the solution evaporated in vacuo and the residue purified by column chromatography upon silica gel using CH₂Cl₂-MeOH-0.880 NH₃ (80:20:5) as eluant. The product was suspended in EtOAc, treated with ethereal HCl, the mixture evaporated in vacuo and the product triturated with EtOAc to afford N-({4-bromo-1-guanidino-7-isoquinolinyl)sulphonyl)-N-[2-(dimethylamino)ethyl]cycloleucine dihydrochloride (15 mg, 0.027 mmol) as a pale yellow solid.

¹H (DMSO-d₆, 300 MHz) δ 1.45-1.6 (4H, m), 1.95 (2H, m), 2.2 (2H, m), 2.6 (6H, s), 3.1 (2H, m), 3.7 (2H, t), 7.35-7.6 (4H, br s), 8.0 (1H, d), 8.15 (1H, d), 8.25 (1H, s), 9.15 (1H, s) ppm.

LRMS 527, 529 (MH⁺)

Anal. Found: C, 41.31; H, 5.35; N, 14.14. Calc. for C₂₀H₂₇BrN₆O₄S.HCl.H₂O: C, 41.27; H, 5.19; N, 14.44%.

### Example 85

### (a) Ethyl 3-{[(4-chloro-1-guanidino-7-isoquinolinyl)sulphonyl]amino}-2,2-dimethylpropanoate hydrochloride

### (b) N-({4-Chloro-1-guanidino-7-isoquinolinyl}sulphonyl)-2,2dimethyl-β-alanine hydrochloride

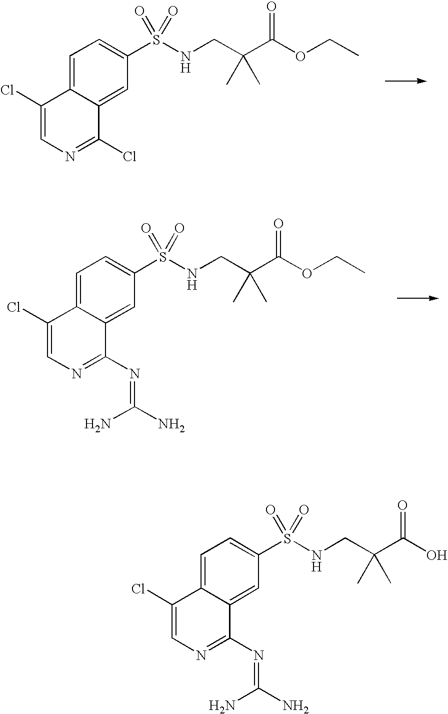

Ethyl 3-{[(4-chloro-1-guanidino-7-isoquinolinyl)sulphonyl]amino}-2,2-dimethylpropanoate hydrochloride was prepared (29%) as a white solid, from ethyl 3-{[(1,4-dichloro-7-isoquinolinyl)sulphonyl]amino}-2,2-dimethylpropanoate, following a similar procedure to that described in example 83.

mp. 183-187° C.

¹H (DMSO-d₆, 300 MHz) δ 1.1 (6H, s), 1.15 (3H, t), 2.95 (2H, d), 4.0 (2H, q), 7.95 (1H, t), 8.35 (1H, m), 8.4 (1H, m), 8.45 (1H, s), 8.5-8.65 (3H, br s), 9.1 (1H, s), 11.2 (1H, s).

LRMS 428 (MH⁺)

Anal. Found: C, 43.99; H, 5.01; N. 14.69. Calc. for C₁₇H₂₂ClN₅O₄S.HCl: C, 43.97; H, 4.99; N, 15.08%.

A solution of ethyl 3-{[(4-chloro-1-guanidino-7-isoquinolinyl)sulphonyl]amino}-2,2-dimethylpropanoate hydrochloride (28 mg, 0.06 mmol) in NaOH solution (2N, 0.5 ml), and MeOH (1 ml), was stirred at 75° C. for 24 h. The cooled mixture was acidified to pH 6 using HCl (2N), concentrated in vacuo to remove the MeOH, and the resulting precipitate filtered, washed with water and dried. The solid was suspended in a MeOH/EtOAc solution, ethereal HCl added, and the mixture evaporated in vacuo to afford N-({4-chloro-1-guanidino-7-isoquinolinyl}sulphonyl)-2,2-dimethyl-β-alanine hydrochloride as a white solid (22 mg, 0.05 mmol).

mp. Dec>304° C.

¹H (DMSO-d₆, 300 MHz) δ 1.05 (6H, s), 2.9 (2H, d), 7.9 (1H, t), 8.3-8.6 (6H, m), 9.05 (1H, s) ppm.

### Example 86

### (a) 1-[({4-Chloro-1-guanidino-7-isoquinolinyl}sulphonyl)amino]-N,N-dimethylcyclopentanecarboxamide

### (b) 1-[({4-Chloro-1-guanidino-7-isoquinolinyl)sulphonyl][2(tetrahydro2H-pyran-2-yloxy)ethyl]amino}-N-N-dimethylcyclopentanecarboxamide

### (c) 1-[({4-Chloro-1-guanidino-7-isoquinolinyl)sulphonyl)(2-hydroxyethyl)amino]-N,N-dimethylcyclopentanecarboxamide hydrochloride

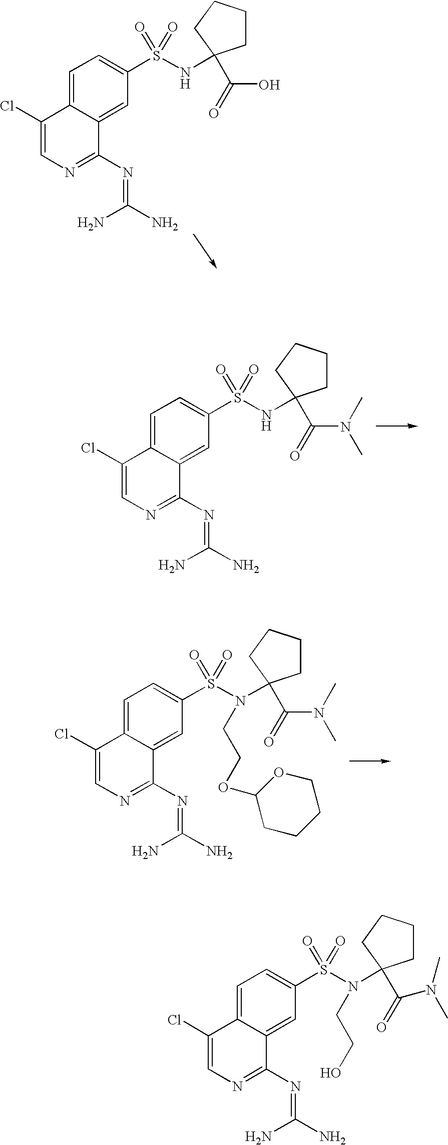

Oxalyl chloride (3.5 ml, 4.0 mmol) was added to a suspension of N-({4-chloro-1-guanidino-7-isoquinolinyl}sulphonyl)cycloleucine hydrochloride (870 mg, 1.94 mmol) in CH₂Cl₂ (100 ml), followed by DMF (5 drops), and the reaction stirred at room temperature for 2 h. The solution was concentrated in vacuo and azeotroped with toluene to give a yellow gum. This was dissolved in CH₂Cl₂ (100 ml), the solution cooled to −20° C., and cooled N,N-dimethylamine (10 ml) added. The reaction was allowed to warm to room temperature with stirring, over 30 min, then concentrated in vacuo, and the residue azeotroped with toluene. The crude product was purified by column chromatography upon silica gel using CH₂Cl₂-MeOH-0.880 NH₃ (95:5:0.5) as eluant, and crystallised from MeOH to afford to afford 1-[({4-chloro-1-guanidino-7-isoquinolinyl}sulphonyl)amino]-N,N-dimethylcyclopentanecarboxamide (302 mg, 0.69 mmol) as a yellow solid.

mp. 264-268° C.

¹H (DMSO-d₆, 400 MHz) δ 1.35 (4H, m), 2.0 (2H, m), 2.2 (2H, m), 3.1 (6H, s), 8.35 (2H, m), 8.4-8.7 (2H, m), 9.1 (1H, s) ppm.

LRMS 439, 441 (MH⁺)

Anal. Found: C, 49.07; H, 5.27; N, 18.51. Calc. for C₁₈H₂₃ClN₆O₃S.0.3H₂O: C, 48.66; H, 5.35; N, 18.91%.

K₂CO₃ (113 mg, 0.82 mmol) was added to a solution of 1-[({4-chloro-1-guanidino-7-isoquinolinyl}sulphonyl)amino]-N,N-dimethylcyclopentanecarboxamide (150 mg, 0.34 mmol) in DMF (2.5 ml), and the mixture heated to 75° C. 2-(2-Bromoethoxy)tetrahydro-2H-pyran (J.C.S. 1948; 4187) (150 mg, 0.72 mmol) and sodium iodide (3 mg) were then added and the reaction stirred at 75° C. for 3 days. The cooled reaction mixture was poured into water, and extracted with EtOAc. The combined organic extracts were washed with brine, dried (Na₂SO₄) and evaporated in vacuo. The residual yellow oil was purified by column chromatography upon silica gel using EtOAc as eluant, and triturated with a hexane-EtOAc (20:1) solution, to give 1-{[(4-chloro-1-guanidino-7-isoquinolinyl)sulphonyl][2-(tetrahydro-2H-pyran-2-yloxy)ethyl]amino)-N-N-dimethylcyclopentanecarboxamide (56 mg, 0.099 mmol).

¹H (CDCl₃, 400 MHz) δ 1.45-1.85 (1H, m), 2.9-3.2 (6H, m), 3.35-3.6 (4H, m), 3.95 (2H, m), 4.1 (1H, m), 4.65 (1H, s), 8.1 (3H, m), 9.25 (1H, s) ppm.

Ethereal HCl was added dropwise to a solution of 1-{[(4-chloro-1-guanidino-7-isoquinolinyl)sulphonyl][2-(tetrahydro-2H-pyran-2-yloxy)ethyl]amino}-N-N-dimethylcyclopentanecarboxamide (37 mg, 0.065 mmol) in EtOAc (1.5 ml), until no further precipitation occurred. The resulting suspension was stirred at room temperature for 20 min, and then evaporated in vacuo. The residue was purified by column chromatography upon silica gel using CH₂Cl₂-MeOH-0.880 NH₃ (95:5:0.5) as eluant, and azeotroped with toluene. This product was dissolved in a MeOH—CH₂Cl₂ solution, ethereal HCl added (5 ml), and the mixture evaporated in vacuo, and triturated with Et₂O to afford 1-[({4-chloro-1-guanidino-7-isoquinolinyl}sulphonyl)(2-hydroxyethyl)amino]-N,N-dimethylcyclopentanecarboxamide hydrochloride (9 mg, 0.017mmol) as a cream/white solid.

¹H (DMSO-d₆+TFA-d, 300 MHz) δ 1.25-1.45 (4H, m), 1.7 (2H, m), 2.25 (2H, m), 2.8-3.0 (6H, m), 3.3 (2H, m), 3.7 (2H, t), 8.35 (1H, d), 8.4 (1H, d), 8.5 (1H, s), 8.6 (1H, br s), 9.0 (1H, s) ppm.

LRMS 483 (MH⁺)

### Example 87

### (a) Ethyl 1-{[(4-chloro-1-guanidino-7-isoquinolinyl)sulphonyl][2-(tetrahydro-2H-pyran-2-yloxy)ethyl}amino]cyclopentanecarboxylate

### (b) 1-{[(4-Chloro-1-guanidino-7-isoquinolinyl)sulphonyl][2-(tetrahydro-2H-pyran-2-yloxy)ethyl]amino}cyclopentanecarboxylic acid

### (c) N′″-(4-Chloro-7-[(10-oxo-9-oxa-6-azaspiro[4.5]dec-6yl)sulphonyl]-1-isoquinolinylguanidine hydrochloride

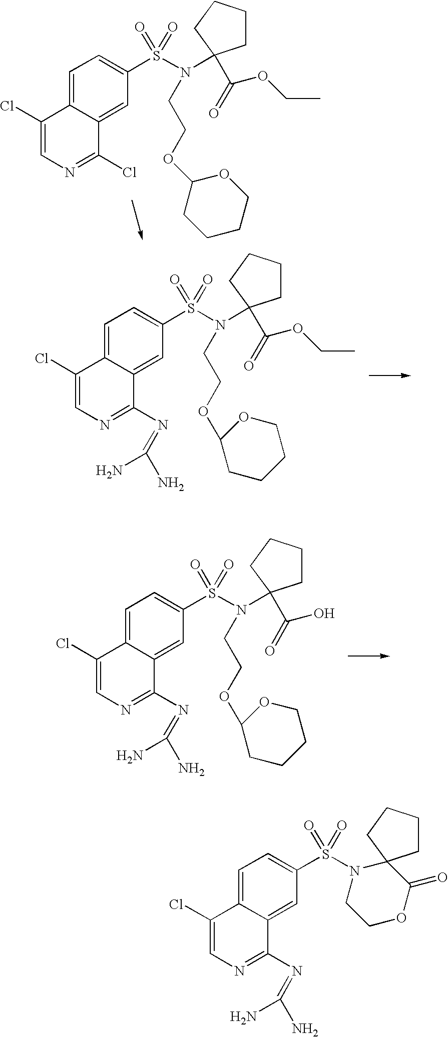

NaH (45 mg, 80% dispersion in mineral oil, 1.5 mmol) was added to a solution of guanidine hydrochloride (231 mg, 2.4 mmol) in DMSO (5 ml), and the solution stirred at 50° C. for 20 min. Ethyl 1-{[(1,4-dichloro-7-isoquinolinyl)sulphonyl][2-(tetrahydro-2H-pyran-2-yloxy)ethyl]amino}cyclopentanecarboxylate (330 mg, 0.6 mmol) was added and the reaction stirred at 70° C. for 2½ h. The cooled reaction was poured into water, extracted with EtOAc, and the combined organic extracts washed with brine, dried (MgSO₄) and evaporated in vacuo. The residual yellow gum was purified by column chromatography upon silica gel using CH₂Cl₂-MeOH-0.880 NH₃ (95:5:0.5) as eluant to give ethyl 1-{[(4-chloro-1-guanidino-7-isoquinolinyl)sulphonyl][2-(tetrahydro-2H-pyran-2-yloxy)ethyl]amino}cyclopentanecarboxylate as an orange oil.

¹H (CDCl₃, 400 MHz) δ 1.25 (3H, t), 1.45-1.75 (14H, m), 2.1 (2H, m), 2.35 (2H, m), 3.5 (1H, m), 3.75-3.9 (4H, m), 4.0 (1H, m), 4.2 (2H, q), 4.61 (1H, s), 8.05-8.15 (3H, m), 9.25 (1H, s), ppm.

LRMS 568 (M⁺)

A solution of ethyl 1-{[(4-chloro-1-guanidino-7-isoquinolinyl)sulphonyl)[2-(tetrahydro-2H-pyran-2-yloxy)ethyl]amino}cyclopentanecarboxylate in MeOH (5 ml), was heated to 75° C., NaOH solution (1 ml, 2N, 2 mmol) added, and the reaction stirred at 50° C. for 48 h. The cooled reaction mixture was concentrated in vacuo, to remove the MeOH, and the remaining aqueous solution acidified to pH 6 using 1N HCl. The resulting precipitate was filtered, washed with water, and the filtrate extracted with EtOAc. The combined organic extracts were dried (MgSO₄), and evaporated in vacuo to give 1-{[(4-chloro-1-guanidino-7-isoquinolinyl)sulphonyl][2-(tetrahydro-2H-pyran-2-yloxy)ethyl]amino}cyclopentanecarboxylic acid (9 mg, 0.017 mmol) as a pale yellow solid.

¹H (CDCl₃, 300 MHz) δ 1.4 (4H, m), 1.55 (4H, m), 2.0 (2H, m), 2.2 (2H, m), 3.35 (3H, m), 3.45-3.75 (5H, m), 4.5 (1H, m), 8.0 (1H, d), 8.15 (2H, m), 9.15 (1H, s) ppm.

Anal. Found: C, 49.50; H, 5.50; N, 12.26. Calc. for C₂₃H₃₀ClN₅O₆S.H₂O: C, 49.50; H, 5.78; N, 12.55%.

1-{[(4-Chloro-1-guanidino-7-isoquinolinyl)sulphonyl][2-(tetrahydro-2H-pyran-2-yloxy)ethyl]amino}cyclopentanecarboxylic acid (20 mg, 0.037 mmol) was dissolved in EtOAc (20 ml), ethereal HCl (10 ml) added, and the reaction stirred at room temperature for 18 h. The resulting precipitate was filtered, washed with EtOAc and dried under vacuum to give N″-{4-Chloro-7-[(10-oxo-9-oxa-6-azaspiro[4.5]dec-6-yl)sulphonyl]-1-isoquinolinyl}guanidine hydrochloride (17 mg, 0.36 mmol).

¹H (CDCl₃, 300 MHz) δ 1.6-1.8 (4H, m), 2.25 (4H, m), 3.95 (2H, t), 4.4 (2H, t), 8.35 (2H, m), 8.45 (1H, s), 9.25 (1H, s), 11.5 (1H, s) ppm.

LRMS 437 (M⁺)

Anal. Found: C, 44.04; H, 4.58; N, 14.17. Calc. for, Cl₈H₂₀ClN₅O₄S.HCl.H₂O: C, 43.91; H, 4.71; N, 14.22%.

### Example 88

### (a) N-1(4-chloro-1-guanidino-7-isoquinolinyl)methyl]cycloleucine methyl ester

### (b) N-({4-Chloro-1-guanidino-7-isoquinolinyl}methyl)cycloleucine dihydrochloride

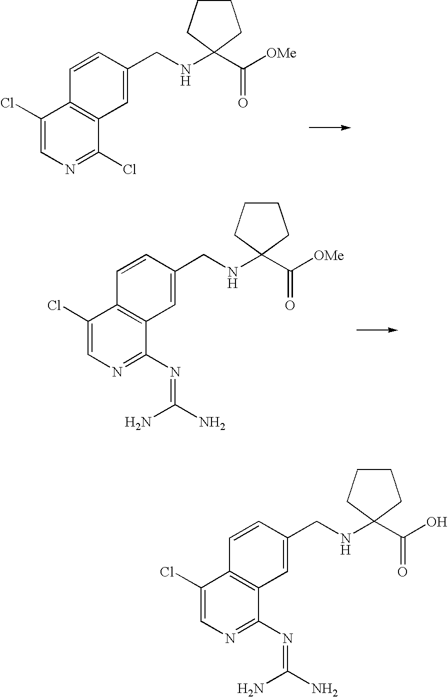

NaH (52 mg, 80% dispersion in mineral oil, 1.73 mmol) was added to a slurry of guanidine hydrochloride (265 mg, 2.77 mmol) in DMSO (2.5 ml) and the mixture heated to 50° C. for 20 mins. N-[(1,4-Dichloro-7-isoquinolinyl)methyl]cycloleucine methyl ester (245 mg, 0.69 mmol) in DMSO (2.5 ml) was added and after heating at 90° C. for 4½ h, the solution was poured into water (50 ml). The mixture was extracted with EtOAc (2×), the combined organic extracts washed with water, brine and then dried (Na₂SO₄). The residue was purified by column chromatography upon silica gel eluting with CH₂Cl₂-MeOH-0.880 NH₃ (90:10:1) to give a yellow solid. This was dissolved in a CH₂Cl₂-MeOH solution and acidified with ethereal HCl (1N), concentrated in vacuo and the crude product recrystallised from EtOH to give N-[(4-chloro-1-guanidino-7-isoquinolinyl)methyl]cycloleucine methyl ester (30 mg, 0.08 mmol) as a cream solid.

mp. 271-275° C.

¹H (DMSO-d₆, 300 MHz) δ 1.25 (3H, t), 1.75 (2H, m), 1.9 (2H, m), 2.1-2.3 (4H, m), 4.25 (2H, q), 4.35 (2H, m), 8.25 (3H, m), 8.4 (1H, s), 9.3 (1H, s), 11.7 (1H, s) ppm.

LRMS 390 (MH⁺)

Anal. Found: C, 49.09; H, 5.74; N, 14.71. Calc. For C₁₉H₂₄ClN₅O₂.2HCl.0.2H₂O: N, 15.02%.

N-[(4-Chloro-1-guanidino-7-isoquinolinyl)methyl]cycloleucine methyl ester (100 mg, 0.27 mmol) was dissolved in methanol (4 ml) at 50° C., NaOH (2N, 1 ml) was added, and the reaction mixture heated for 2 days at 50° C. The cooled mixture was basified to pH 6 with NaOH (2N) to give a precipitate which was filtered off and washed with water. The solid was dissolved in MeOH/EtOAc, acidified with ethereal HCl (1N) and triturated with i-Pr₂O to give the title compound (b) as a pale yellow solid (10 mg, 0.03 mmol).

mp 281-289° C.

¹H (DMSO-d₆+TFA-d, 300 MHz) δ 1.8 (2H, m), 1.85 (2H, m), 2.15 (2H, m), 2.25 (2H, m), 4.4 (2H, s), 8.2 (1H, d), 8.3 (1H, d), 8.4 (1H, s), 9.15 (1H, s) ppm.

LRMS 362 (MH⁺).

### PREPARATIONS

### Preparation 1

### 7-Bromo-1,4-dichloroisoquinoline

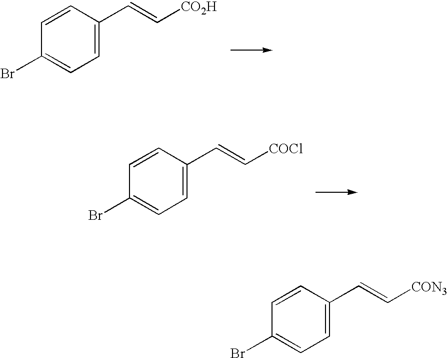

A solution of 4-bromocinnamic acid (5.03 g, 22.2 mmol) in SOCl₂ (15 mL) was stirred at 23° C. for 16 h, and then heated at reflux for a further 2 h. The solvents were evaporated in vacuo and the residue azeotroped with PhMe (×3) to yield 4-bromocinnamoyl chloride (22 mmol) as an orange-brown solid.

¹H NMR (CDCl₃, 300 MHz) δ 6.65 (1H, d), 7.4 (2H, d), 7.6 (2H, d), 7.8 (1H, d) ppm.

A solution of NaN₃ (2.2 g, 33.8 mmol) in water (7.5 mL) was added dropwise over 5 min to a stirred solution of 4-bromocinnamoyl chloride (22 mmol) in acetone (22 mL) at −10° C. The heterogeneous mixture was stirred at 0° C. for 1 h and diluted with water (25 mL). The precipitate was collected by filtration and dried in vacuo over P₂O₅ to give 4-bromocinnamoyl azide (5.22 g, 20.7 mmol) as a golden-coloured solid.

¹H NMR (CDCl₃, 300 MHz) δ 6.4 (1H, d), 7.4 (2H, d), 7.5 (2H, d), 7.65 (1H, d) ppm.

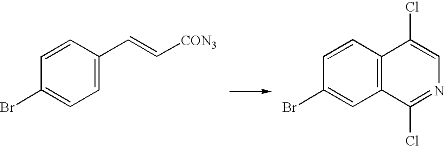

A warm solution of 4-bromocinnamoyl azide (5.22 g, 20.7 mmol) in Ph₂O (25 mL) was added dropwise over 15 min to stirred Ph₂O (10 mL) at 270° C. [CAUTION: Potentially explosive—use a blast screen.] The mixture was heated at 270° C. for 1.5 h, cooled to 23° C. and then poured into hexanes (400 mL). The precipitate was collected by filtration, with hexanes (2×100 mL) rinsing, and purified by column chromatography upon silica gel using hexanes-EtOAc (6:4 to 0:100) as eluant to give 7-bromo-1(2H)-isoquinolone (1.64 g, 7.3 mmol) as a white solid.

¹H NMR (DMSO-d₆, 300 MHz) δ 6.55 (1H, d), 7.25-7.15 (1H, m), 7.6 (1H, d), 7.8 (1H, d), 8.25 (1H, s), 11.4 (1H, br s) ppm.

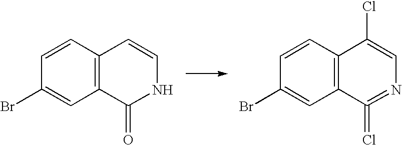

A mixture of 7-bromo-1(2H)-isoquinolone (1.28 g, 5.69 mmol) and PCl₅ (2.04 g, 9.80 mmol) was heated at 140° C. for 5 h. The cooled mixture was quenched with ice (50 g) and 0.880NH₃ was added until alkaline by litmus paper. The aqueous mixture was extracted with CH₂Cl₂ (3×50 mL) and the combined organic phases were dried (MgSO₄) and evaporated in vacuo. The residue was purified by column chromatography upon silica gel using hexanes-EtOAc (97:3 to 95:5) as eluant to give 7-bromo-1,4-dichloroisoquinoline (1.13 g, 4.08 mmol) as a white solid.

mp 133.5-135° C.

¹H (CDCl₃, 300 MHz) δ 7.9 (1H, d), 8.1 (1H, d), 8.35 (1H, s), 8.5 (1H, s).

LRMS 276, 278 (MH⁺).

Anal. Found: C, 39.04; H, 1.32; N, 5.06. Calc for C₉H₄BrCl₂N: C, 39.03, H, 1.46; N, 5.06.

### Preparation 2

### t-Butyl 2-aminobenzoate

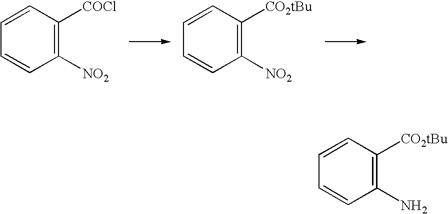

A mixture of 2-nitrobenzoyl chloride (15 mL, 110 mmol) and t-BuOH (100 mL) were heated at reflux for 3 h. The cooled mixture was poured onto ice-water, basified with Na₂CO₃ and extracted with CH₂Cl₂ (×2). The combined organic extracts were washed with brine, the solvents evaporated in vacuo and the residue was purified by column chromatography upon silica gel using hexanes-EtOAc (95:5) as eluant to give t-butyl 2-nitrobenzoate (4.9 g, 22 mmol) as a yellow oil.

¹H (CDCl₃, 300 MHz) δ 1.6 (9H, s), 7.5 (1H, dd), 7.6 (1H, dd), 7.7 (1H, d), 7.8 (1H, d) ppm.

LRMS 240 (MNH₄⁺).

A solution of t-butyl 2-nitrobenzoate (4.9 g, 22 mmol) in EtOH (160 mL) was stirred with 10% palladium-carbon (700 mg) under an atmosphere of H₂ (60 psi) at 23° C. After 4 h, the mixture was filtered and evaporated in vacuo to give t-butyl 2-aminobenzoate (4.0 g, 20.7 mmol) as a yellow oil.

¹H (CDCl₃, 300 MHz) δ 1.6 (9H, s), 5.6-5.8 (2H, br s), 6.6 (1H, dd), 6.6 (1H, d), 7.2 (1H, dd), 7.8 (1H, d) ppm.

LRMS 194 (MH⁺).

### Preparation 3

### t-Butyl 2-{[(1,4-dichloro-7-isoquinolinyl)sulphonyl]amino}benzoate

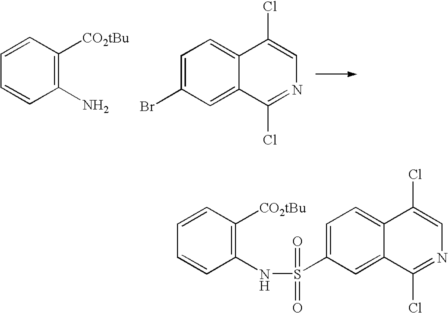

n-Butyllithium (0.88 mL, 2.5 M in hexanes, 2.2 mmol) was added dropwise to a stirred solution of 7-bromo-1,4-dichloroisoquinoline (570 mg, 2.0 mmol) in THF-Et₂O (10 mL, 1:1) under N₂ at -78° C. After 5 min, the mixture was added to a solution of SO₂Cl₂ (0.35 mL, 4.35 mmol) in hexane (10 mL) −78° C. under N₂, and the mixture was slowly warmed to 23° C. and then stirred for 4.5 h. The solvents were evaporated in vacuo, azeotroping with CH₂Cl₂ and PhMe, the residue was suspended in CH₂Cl₂ (12 mL) containing NEt₃ (1.15 mL, 8.25 mmol) and t-butyl 2-aminobenzoate (520 mg, 2.7 mmol) was added. The mixture was stirred at room temperature for 3 d and then heated at reflux for 6 h. The cooled mixture was diluted with CH₂Cl₂, washed with aqueous HCl (2 M), brine, and then evaporated in vacuo. The residue was purified by column chromatography upon silica gel using hexanes-EtOAc (97:3 to 95:5) as eluant to give, initially, 1,4,7-trichloroisoquinoline (200 mg) followed by t-butyl 2-{[(1,4-dichloro-7-isoquinolinyl)sulphonyl]amino}benzoate (120 mg, 0.26 mmol) as a yellow resin.

¹H (CDCl₃, 400 MHz) δ 1.5 (9H, s), 7.05 (1H, dd), 7.5 (1H, dd), 7.7 (1H, d), 7.8 (1H, d), 8.2 (1H, d), 8.3 (1H, d), 8.4 (1H, s), 8.8 (1H, s), 10.0 (1H, s) ppm.

LRMS 454 (MH⁺).

### Preparation 4

### t-Butyl 3-aminobenzoate

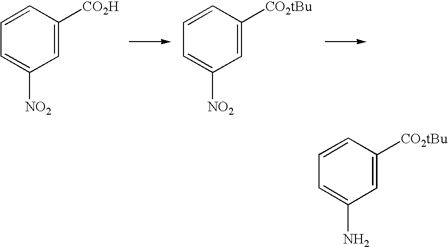

A mixture of 3-nitrobenzoic acid (5 g, 30 mmol), di-tert-butyl dicarbonate (20 g, 92 mmol), and DMAP (0.84 g, 6.9 mmol) in THF (60 mL) was stirred at 23° C. for 2 d. The mixture was poured onto ice-water, basified with Na₂CO₃ and extracted with CH₂Cl₂ (×3). The combined organic extracts were washed with brine, the solvents evaporated in vacuo and the residue was purified by column chromatography upon silica gel using hexanes-EtOAc (95:5) as eluant to give t-butyl 3-nitrobenzoate (5.4 g, 24 mmol) as a colourless oil.

¹H (CDCl₃, 400 MHz) δ 1.4 (9H, s), 7.6 (1H, dd), 8.3 (1H, d), 8.4 (1H, d), 8.8 (1H, s) ppm.

A solution of t-butyl 3-nitrobenzoate (5.8 g, 26 mmol) in EtOH (260 mL) was stirred with 10% palladium-carbon (1.0 g) under an atmosphere of H₂ (60 psi) at 23° C. After 4 h, the mixture was filtered and evaporated in vacuo to give t-butyl 3-aminobenzoate (4.0 g, 20.7 mmol) as a white solid.

¹H (CDCl₃, 400 MHz) δ 1.6 (9H, s), 3.6-3.9 (2H, br s), 6.8 (1H, d), 7.2 (1H, dd), 7.3 (1H, s), 7.4 (1H, d) ppm.

LRMS 194 (MH⁺), 387 (M₂K⁺).

### Preparation 5

### t-Butyl 3-{[(1,4-dichloro-7-isoquinolinyl)sulphonyl]amino}benzoate

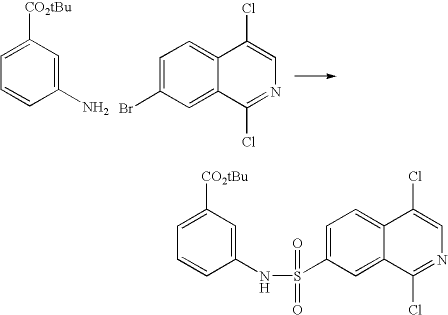

n-Butyllithium (0.88 mL, 2.5 M in hexanes, 2.2 mmol) was added dropwise to a stirred solution of 7-bromo-1,4-dichloroisoquinoline (570 mg, 2.0 mmol) in THF-Et₂O (10 mL, 1:1) under N₂ at −78° C. After 5 min, the mixture was added to a solution of SO₂Cl₂ (0.35 mL, 4.35 mmol) in hexane (10 mL) at −78° C. under N₂, and the mixture was slowly warmed to 23° C. and then stirred for 4.5 h. The solvents were evaporated in vacuo, azeotroping with PhMe, the residue was suspended in CH₂Cl₂ (12 mL) and t-butyl 3-aminobenzoate (520 mg, 2.7 mmol) followed by NEt₃ (1.15 mL, 8.25 mmol) were added. The mixture was stirred at room temperature for 4 d and evaporated in vacuo. The residue was purified by column chromatography upon silica gel using hexanes-EtOAc (90:10 to 50:50) as eluant to give, initially, 1,4,7-trichloroisoquinoline (150 mg) followed by t-butyl 2-{[(1,4-dichloro-7-isoquinolinyl)sulphonyl]amino)benzoate (289 mg, 0.63 mmol) as a brown solid which was used without further purification.

¹H (CDCl₃, 400 MHz) selected data: δ 1.5 (9H, s), 7.20-7.25 (1H, m), 7.3-7.45 (1H, m), 7.5 (1H, dd), 7.6 (1H, s), 8.45 (1H, d), 8.5 (1H, d), 8.6 (1H, s), 8.9 (1H, s) ppm.

LRMS 454 (MH⁺).

### Preparation 6

### 1,4-Dichloro-7-isoquinolinesulphonyl chloride

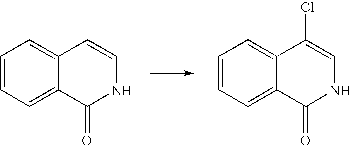

A solution of N-chlorosuccinimide (9.66 g, 72 mmol) in MeCN (80 mL) was added dropwise to a stirred solution of 1-(2H)-isoquinolone (10 g, 69 mmol) in MeCN (250 mL) which was being heated under reflux. The mixture was heated under reflux for an additional 1.5 h and then cooled to room temperature. The resulting precipitate was collected by filtration, with MeCN rinsing, and then dried in vacuo to give 4-chloro-1(2H)-isoquinolone (11.3 g, 62.9 mmol) as a pale pink solid.

¹H (DMSO-d₆, 300 MHz) δ 7.5 (1H, s), 7.6 (1H, dd), 7.8-7.9 (2H, m), 8.25 (1H, d), 11.5 (1H, br s), ppm.

LRMS 180,182 (MH⁺), 359, 361,363 (M₂H⁺).

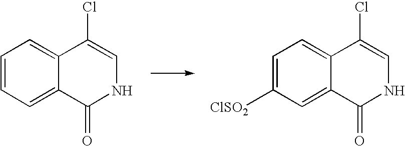

4-Chloro-1-(2H)-isoquinolone (20.62 g, 115 mmol) was added portionwise to stirred chlorosulphonic acid (61 mL, 918 mmol) at 0° C. The mixture was heated at 100° C. for 3.5 d and then cooled to room temperature. The reaction mixture was added in small portions onto ice-water [CAUTION] and the resulting precipitate was collected by filtration. The solid was washed with water, triturated with MeCN and then dried in vacuo to give 4-chloro-1-oxo-1,2-dihydro-7-isoquinolinesulphonyl chloride (18.75 g, 67.4 mmol) as a cream solid.

¹H (DMSO-d₆, 400 MHz) δ 7.45 (1H, s), 7.8 (1H, d), 8.0 (1H, d), 8.5 (1H, s), 11.5 (1H, br s) ppm.

Anal. Found: C, 39.37; H, 2.09; N, 4.94. Calc for C₉H₅Cl₂NO₃S: C, 38.87; H, 1.81; N, 5,04.

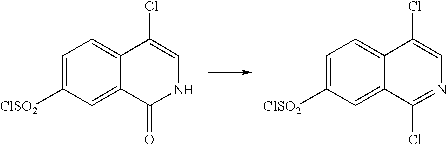

POCl₃ (9.65 mL, 103.5 mmol) was added to a stirred suspension of 4-chloro-1-oxo-1,2-dihydro-7-isoquinolinesulphonyl chloride (22.1 g, 79.6 mmol) in MeCN (500 mL) at room temperature and the mixture was then heated at reflux for 15 h. On cooling, the MeCN solution was decanted from the insoluble sludge and evaporated in vacuo. The residue was extracted with hot EtOAc and evaporated to leave a solid which was stirred with Et₂O (1.2 L) at room temperature overnight. The ethereal solution was decanted from the insoluble material and evaporated in vacuo to give 1,4-dichloro-7-isoquinolinesulphonyl chloride (20 g, 67 mmol) as a pale yellow solid.

¹H (DMSO-d₆, 400 MHz) δ 8.2 (2H, s), 8.5 (1H, s), 8.55 (1H, s) ppm.

Anal. Found: C, 37.19; H, 1.34; N, 4.77. Calc for C₉H₄Cl₃NO₂S: C, 36.45; H, 1.36; N, 4.72.

### Preparation 7

### Methyl 3-{[(1,4-dichloro-7-isoquinolinyl)sulphonyl]amino}-4-methoxyberzoate

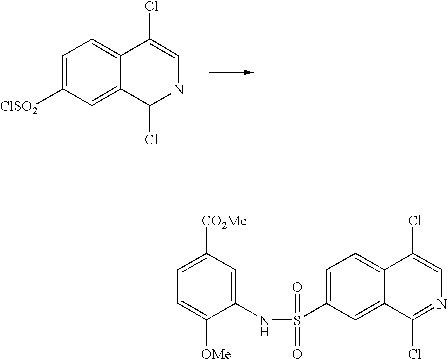

Methyl 3-amino-4-methoxybenzoate (212 mg, 1.17 mmol) was added to a stirred solution of 1,4-dichloro-7-isoquinolinesulphonyl chloride (342 mg, 1.15 mmol) in CH₂Cl₂ (10 mL) containing 2,6-lutidine (0.135 mL, 1.16 mmol) under N₂ at 0° C. After 5 min, the mixture was warmed to room temperature and stirred for 22 h. The solvents were evaporated in vacuo and the residue was suspended in EtOAc (50 mL), and then washed with water, brine, dried (MgSO₄) and evaporated in vacuo. The residue was purified by column chromatography upon silica gel using hexanes-EtOAc (80:20 to 20:80) as eluant to give methyl 3-1{[(1,4-dichloro-7-isoquinolinyl)sulphonyl]amino}-4-methoxybenzoate (365 mg, 0.83 mmol) as an off-white solid.

¹H (CDCl₃, 300 MHz) δ 3.7 (3H, s), 3.9 (3H, s), 6.75 (1H, d), 7.2 (1H, s), 7.8 (1H, dd), 8.15 (1H, dd), 8.25 (1H, s), 8.3 (1H, d), 8.5 (s, 1H), 8.85 (1H, s) ppm.

LRMS 441 (MH⁺), 458 (MNH₄⁺).

### Preparation 8

### N-[(1,4-Dichloro-7-isoquinolinyl)sulphonyl]glycine t-butyl ester

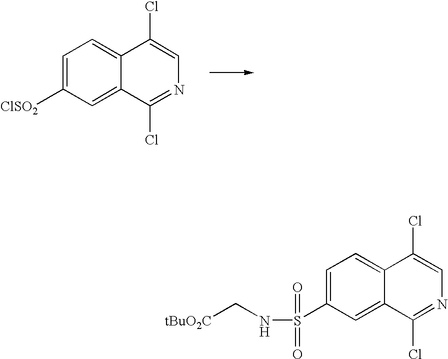

NEt₃ (0.59 mL, 4.24 mmol) was added to a stirred solution of glycine t-butyl ester hydrochloride (340 mg, 2.02 mmol) and 1,4-dichloro-7-isoquinolinesulphonyl chloride (500 mg, 1.68 mmol) in CH₂Cl₂ (25 mL) under N₂ and the mixture was stirred at room temperature for 18 h. The mixture was diluted with CH₂Cl₂ (25 mL), washed with dilute HCl (×2, 1 M), saturated aqueous NaHCO₃, brine, dried (MgSO₄) and evaporated ill vacuo. The solid was triturated with EtOAc, collected by filtration and dried to give N-[(1,4-dichloro-7-isoquinolinyl)sulphonyl]glycine t-butyl ester (435 mg, 1.11 mmol) as a white solid.

mp 194-196° C.

¹H (CDCl₃, 300 MHz) δ 1.3 (9H, s), 3.8 (2H, d), 5.3 (1H, br t), 8.25 (1H, d), 8.4 (1H, d), 8.5 (1H, s), 8.9 (1H, s) ppm.

LRMS 391 (MH⁺), 408, 410 (MNH₄⁺).

Anal. Found: C, 45.58; H, 4.03; N, 7.03. Calc for C₁₅H₁₆C₁₂N₂O₄S: C, 46.04; H, 4.12; N, 7.16.

### Preparation 9

### N-[(1,4-Dichloro-7-isoquinolinyl)sulphonyl)-β-alanine t-butyl ester

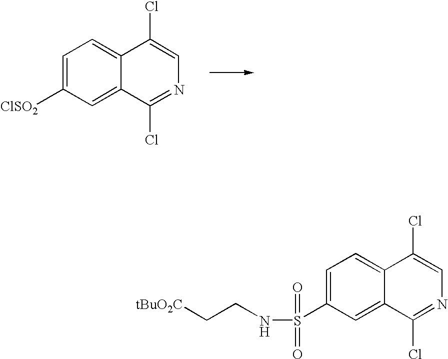

NEt₃ (0.60 mL, 4.3 mmol) was added to a stirred solution of β-alanine t-butyl ester hydrochloride (331 mg, 1.82 mmol) and 1,4-dichloro-7-isoquinolinesulphonyl chloride (510 mg, 1.72 mmol) in CH₂Cl₂ (10 mL) under N₂ and the mixture was stirred at room temperature for 22 h. The mixture was diluted with CH₂Cl₂ (50 mL), washed with half saturated brine, dried (MgSO₄) and evaporated in vacuo. The residue was purified by column chromatography upon silica gel using pentane-EtOAc (90:10 to 60:40) as eluant to give N-[(1,4-dichloro-7-isoquinolinyl)sulphonyl]-β-alanine t-butyl ester (580 mg, 1.43 mmol) as a white solid.

¹H (CDCl₃, 300 MHz) δ 1.4 (9H, s), 2.5 (2H, t), 3.25 (2H, dt), 5.5 (1H, br t), 8.25 (1H, d), 8.4 (1H, d), 8.5 (1H, s), 8.9 (1H, s) ppm.

LRMS 405, 407 (MH⁺), 422 (MNH₄⁺).

Anal. Found: C, 47.41; H, 4.46; N, 6.80. Calc for C16H₁₈Cl₂N₂O₄S: C, 47.42; H, 4.48; N, 6.91.

### Preparation 10

### N-[(1,4-Dichloro-7-isoquinolinyl)sulphonyl]-N-methylglycine t-butyl ester

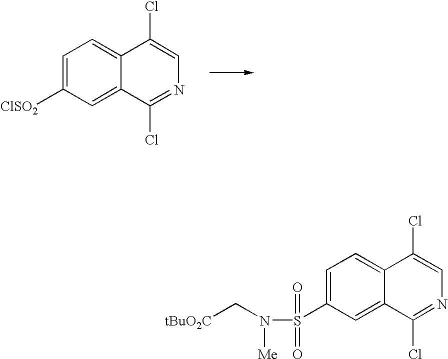

N-Methylglycine t-butyl ester hydrochloride (264 mg, 1.45 mmol) was added to a stirred solution of 1,4-dichloro-7-isoquinolinesulphonyl chloride (376 mg, 1.27 mmol) in CH₂Cl₂ (25 mL) containing NEt₃ (0.44 mL, 3.16 mmol) under N₂ at 0° C., and the mixture was then stirred at room temperature for 22 h. The solvents were evaporated in vacuo, the residue dissolved in EtOAc (50 mL), washed with water, brine, dried (MgSO₄) and evaporated in vacuo. The residue was purified by column chromatography upon silica gel using pentanes-EtOAc (80:20) as eluant to give N-[(1,4-dichloro-7-isoquinolinyl)sulphonyl]-N-methylglycine t-butyl ester (485 mg, 1.20 mmol) as a white solid.

¹H (CDCl₃, 300 MHz) δ 1.35 (9H, s), 3.0 (3H, s), 4.05 (2H, d), 8.2 (1H, d), 8.35 (1H, d), 8.5 (1H, s), 8.85 (1H, s) ppm.

LRMS 709 (M₂H⁺).

Anal. Found: C, 47.37; H, 4.43; N, 6.79. Calc for C₁₆H₁₈Cl₂N₂O₄S: C, 47.42; H, 4.48; N, 6.91.

### Preparation 11

### N-Phenylglycine t-butyl ester

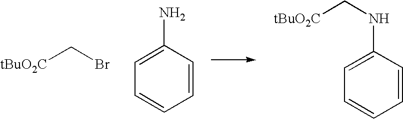

t-Butyl chloroacetate (10 g, 66.3 mmol) was added dropwise to a stirred solution of aniline (11.3 g, 120 mmol) in NEt₃ (10 mL), and the mixture was stirred at to room temperature for 24 h and then at 60° C. for 18 h. The cooled mixture was diluted with Et₂O (100 mL), filtered with Et₂O rinsing, and the filtrate was then washed with water, brine, dried (MgSO₄) and evaporated in vacuo. The residue was purified by column chromatography upon silica gel using hexanes-EtOAc (98:2 to 92:8) as eluant to give N-phenylglycine t-butyl ester (6.56 g, 31.6mmol) as an oil.

¹H (CDCl₃, 400 MHz) δ 1.5 (9H, s), 3.8 (2H, s), 4.45 (1H, br s), 6.6 (2H, d), 6.7 (1H, t), 7.2 (2H, dd) ppm.

LRMS 208 (MH⁺), 415 (M₂H⁺).

### Preparation 12

### N-[(1,4-Dichloro-7-isoquinolinyl)sulphonyl]-N-phenylglycine t-butyl ester

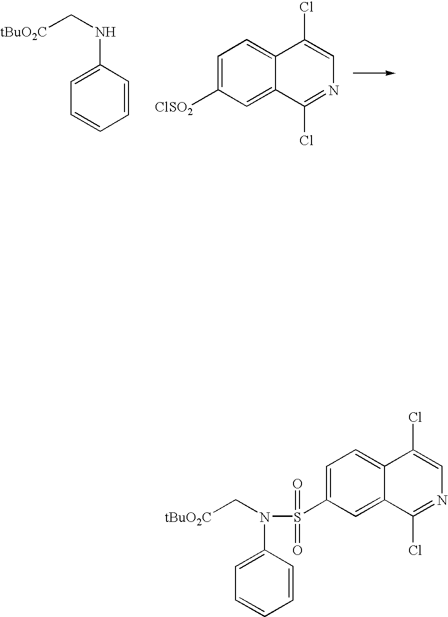

1,4-Dichloro-7-isoquinolinesulphonyl chloride (300 mg, 1.01 mmol) was added to a stirred solution of N-phenylglycine t-butyl ester (228 mg, 1.10 mmol) in CH₂Cl₂ (5.0 mL) containing NEt₃ (0.35 mL, 2.5 mmol) under N₂ at room temperature, and the mixture stirred for 5 d. The mixture was diluted with CH₂Cl₂ (50 mL), washed with dilute HCl (20 mL, 1 M), saturated aqueous NaHCO₃, dried (MgSO₄) and evaporated in vacuo. The residue was purified by column chromatography upon silica gel using hexanes-EtOAc (90:10 to 60:40) as eluant to give N-[(1,4-dichloro-7-isoquinolinyl)sulphonyl]-N-phenylglycine t-butyl ester (485 mg, 1.20 mmol) as a white solid.

¹H (CDCl₃, 300 MHz) δ 1.4 (9H, s), 4.4 (2H, d), 7.2-7.4 (5H, m), 8.05 (1H, d), 8.3 (1H, d), 8.45 (1H, s), 8.7 (1H, s) ppm.

LRMS 467 (MH⁺).

### Preparation 13

### N-(Cyclopentylmethyl)-N-[(1,4-dichloro-7-isoquinolinyl)sulphonyl]glycine t-butyl ester

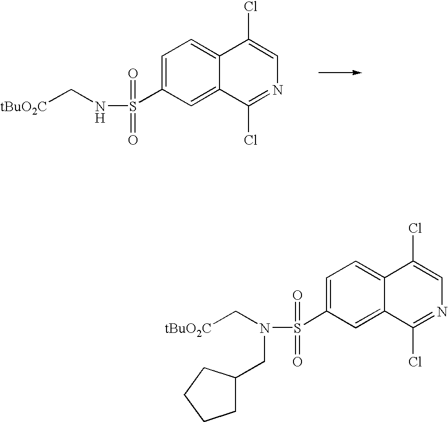

PPh₃ (243 mg, 1.5 mmol) and then a solution of DEAD (236 μL, 1.5 mmol) in THF (2 mL) were added to a stirred solution of N-[(1,4-dichloro-7-isoquinolinyl)sulphonyl]glycine t-butyl ester (391 mg, 1.00 mmol) and cyclopentanemethanol (130 μL, 1.2 mmol) in THF (3 mL) under N₂ at 0° C., and the mixture was stirred at room temperature for 18 h. An additional portion of cyclopentanemethanol (1.2 mmol), PPh₃ (1.5 mmol), and DEAD (1.5 mmol) were added and the mixture stirred at room temperature for a further 2 d. The solvents were evaporated in vacuo and the residue was purified by column chromatography upon silica gel using pentane-EtOAc (100:0 to 95:5) as eluant to give N-(cyclopentylmethyl)-N-[(1.4-dichloro-7-isoquinolinyl)sulphonyl]glycine t-butyl ester (144 mg, 0.30 mmol) as a white solid.

¹H (CDCl₃, 400 MHz) δ 1.15-1.4 (3H, m), 1.3 (9H, s), 1.5-1.7 (3H, m), 1.7-1.8 (2H, m), 2.1 (1H, m), 3.25 (2H, d), 4.1 (2H, s), 8.25 (1H, d), 8.35 (1H, d), 8.5 (1H, s), 8.85 (1H, s) ppm.

LRMS 473 (MH⁺), 490, 492 (MNH₄⁺).

Anal. Found: C, 53.23; H, 5.58; N, 5.86. Calc for C₂₁H₂₆Cl₂N₂₀₄S: C, 53.28; H, 5.54; N, 5.92.

### Preparation 14

### N-(Cyclohexylmethyl)-N-[(1,4-dichloro-7-isoquinolinyl)sulphonyl]glycine t-butyl ester

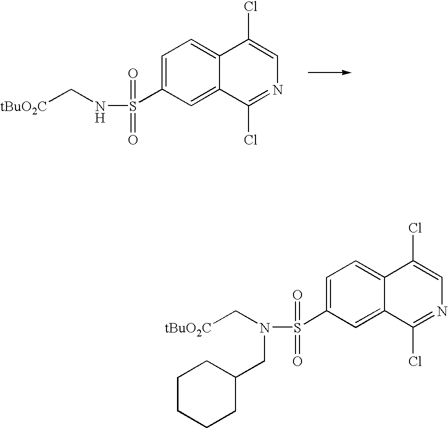

Cyclohexylmethyl bromide (209 μL, 1.5 mmol) was added to a stirred solution of N-[(1,4-dichloro-7-isoquinolinyl)sulphonyl]glycine t-butyl ester (391 mg, 1.00 mmol) and anhydrous K₂CO₃ (276 mg, 2.0 mmol) in DMF (5 mL) under N. at 23° C. The mixture was stirred for 2 h and then heated at 50-60° C. for 6 h. The cooled mixture was diluted with EtOAc (200 mL), washed with water (250 mL), dried (MgSO₄), and the solvents were evaporated in vacuo. The residue was purified by column chromatography upon silica gel using pentane-EtOAc (100:0 to 95:5) as eluant to give N-(cyclohexylmethyl)-N-[(1,4-dichloro-7-isoquinolinyl)sulphonyl]glycine t-butyl ester (320 mg, 0.66 mmol).

¹H (CDCl₃, 400 MHz) δ 1.15-1.3 (3H, m), 1.3 (9H, s), 1.5-1.8 (8H, m), 3.15 (2H, d), 4.05 (2H, s), 8.2 (1H, d), 8.35 (1H, d), 8.45 (1H, s), 8.85 (1H, s) ppm.

LRMS 487 (MH⁺), 504, 506, 508 (MNH₄⁺).

### Preparation 15

### N-Benzylglycine t-butyl ester

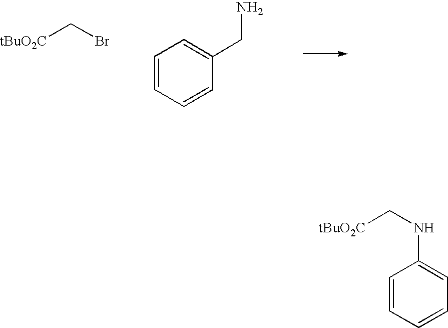

A solution of t-butyl bromoacetate (1.5 mL, 10.1 mmol) in CH₂Cl₂ (10 mL) was added dropwise to a stirred solution of benzylamine (10.9 mL, 100 mmol) in CH₂Cl₂ (40 mL) at 0° C., the mixture was stirred for 1 h and then warmed to room temperature and stirred for an additional 3 d. The mixture was washed with water (3×50 mL), dilute HCl (1 N) and the combined aqueous washings were extracted with Et₂O. The organic phase was washed with saturated aqueous NaHCO₃, dried (Na₂SO₄) and evaporated in vacuo. The residue was dissolved in Et₂O, treated with a solution of HCl in ether (0.5 M) and the resulting precipitate was collected and dissolved in EtOAc. This solution was filtered through hyflo, and partially evaporated in vacuo to give a thick slurry. The solid was collected by filtration, washed with Et₂O and then dried to give N-benzylglycine t-butyl ester hydrochloride (1.03 g, 4.00 mmol) as a white solid.

¹H (CDCl₃, 300 MHz) δ 1.4 (9H, s), 3.5 (2H, s), 4.4 (2H, s), 7.3-7.4 (3H, m), 7.55-7.65 (2H, m), 10.2-10.3 (2H, br s).

LRMS 222, (MH⁺), 443 (M₂H⁺).

### Preparation 16

### N-[(1,4-Dichloro-7-isoquinolinyl)sulphonyl]-N-benzylglycine t-butyl ester

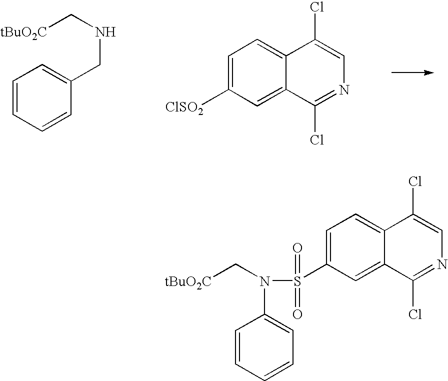

1,4-Dichloro-7-isoquinolinesulphonyl chloride (300 mg, 1.01 mmol) was added to a stirred solution of N-benzylglycine t-butyl ester (310 mg, 1.20 mmol) in CH₂Cl₂ (20 mL) containing NEt₃ (0.35 mL, 2.5 mmol) under N₂ and the mixture was stirred at room temperature for 3 d. The mixture was diluted with CH₂Cl₂ and washed with dilute HCl (2 M), saturated aqueous NaHCO₃, brine, dried (Na₂SO₄) then and evaporated in vacuo. The residue was purified by column chromatography upon silica gel using hexanes-EtOAc (90:10) as eluant to give N-[(1,4-dichloro-7-isoquinolinyl)sulphonyl]-N-benzylglycine t-butyl ester (290 mg, 0.60 mmol) as an off-white solid.

mp 134-136° C.

¹H (CDCl₃, 400 MHz) δ 1.3 (9H, s), 3.9 (2H, s), 4.55 (2H, s), 7.25-7.4 (5H, m), 8.25 (1H, d), 8.4 (1H, d), 8.5 (1H, s), 8.9 (1H, s) ppm.

LRMS 481 (MH⁺), 498 (MNH₄⁺).

Anal. Found: C, 54.52; H, 4.50; N, 5.77. Calc for C₂₂H₂₂Cl₂N₂O₄S: C, 54.89; H, 4.61; N, 5.82.

### Preparation 17

### N-(2-Methylbenzyl)glycine t-butyl ester

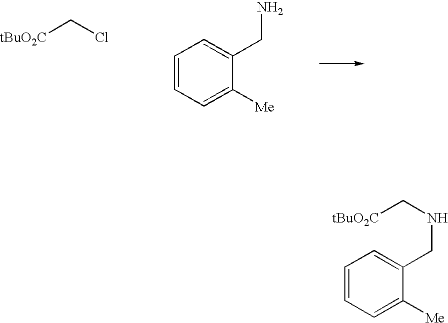

t-Butyl chloroacetate (2.13 g, 14.1 mmol) was added to a stirred solution of 2-methylbenzylamine (1.71 g, 14.1 mmol) in CH₂Cl₂ (20 mL) containing NEt₃ (2.95 mL, 21.2 mmol) under N₂ and the mixture was stirred at room temperature for 17 h. The solvents were evaporated in vacuo, the residue suspended in EtOAc and and washed with water, brine, dried (MgSO₄) then and evaporated in vacuo. The residue was purified by column chromatography upon silica gel using pentanes-EtOAc (95:5 to 80:20) as eluant to give N-(2-methylbenzyl)glycine t-butyl ester (1.29 g, 5.48 mmol).

¹H (CDCl₃, 300 MHz) δ 1.5 (9H, s), 2.35 (3H, s), 3.3 (2H, s), 3.8 (2H, s), 7.1-7.2 (3H, m), 7.25-7.3 (1H, m) ppm.

LRMS 236 (MH⁻), 471 (M₂H⁺).

### Preparation 18

### N-[(1,4-Dichloro-7-isoquinolinyl)sulphonyl]-N-(2-methylbenzyl)glycine t-butyl ester

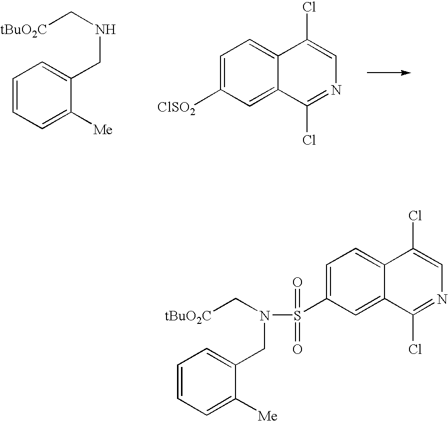

1,4-Dichloro-7-isoquinolinesulphonyl chloride (400 mg, 1.35 mmol) was added to a stirred solution of N-(2-methylbenzyl)glycine t-butyl ester (380 mg, 1.61 mmol) in CH₂Cl₂ (20 mL) containing NEt₃ (0.28 mL, 2.5 mmol) under N₂ and the mixture was stirred at room temperature for 18 h. The mixture was diluted with CH₂Cl₂ and washed with dilute HCl (2 M), saturated aqueous NaHCO₃, brine, dried (MgSO₄) then and evaporated in vacuo. The residue was purified by column chromatography upon silica gel using pentane-EtOAc (100:0 to 90:10) as eluant to give N-[(1,4-dichloro-7-isoquinolinyl)sulphonyl]-N-(2-methylbenzyl)glycine t-butyl ester (480 mg, 0.97 mmol) as a white solid.

mp 96-98° C.

¹H (CDCl₃, 400 MHz) δ 1.25 (9H, s), 2.3 (3H, s), 3.9 (2H, s), 4.6 (2H, s), 7.1-7.25 (4H, m), 8.3 (1H, d), 8.4 (1H, d), 8.5 (1H, s), 8.9 (1H, s) ppm.

LRMS 495 (MH⁺), 512 (MNH₄⁺).

Anal. Found: C, 55.70; H, 4.86; N, 5.63. Calc for C₂₃H₂₄Cl₂N₂O₄S: C, 55.76; H, 4.88; N, 5.65.

### Preparation 19

### N-(2-Methoxybenzyl)glycine t-butyl ester

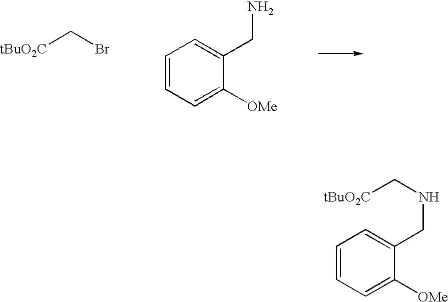

A solution of t-butyl bromooacetate (1.5 mL, 10.2 mmol) in CH₂Cl₂ (30 mL) was added to a stirred solution of 2-methoxybenzylamine (6.88 g, 50.2 mmol) in CH₂Cl₂ (70 mL) under N₂ at 0° C., and the mixture was then stirred at room temperature for 1 h. The mixture was thoroughly washed with dilute HCl (30 mL, 1 M) and the separated aqueous phase was extracted with in CH₂Cl₂. The combined organic extracts were washed with saturated NaHCO₃, brine, dried (Na₂SO₄) then and evaporated in vacuo. The residue was purified by column chromatography upon silica gel using in CH₂Cl₂-MeOH (99:1 to 95:5) as eluant to give N-(2-methoxybenzyl)glycine t-butyl ester (0.90 g, 3.58 mmol) as a pale yellow oil.

¹H (CDCl₃, 400 MHz) δ 1.25 (9H, s), 2.0 (1H, br s), 3.3 (2H, s), 3.8 (2H, s), 3.85 (3H, s), 6.85 (1H, d), 6.9 (1H, dd), 7.2-7.3 (2H, m) ppm.

LRMS 252 (MH⁺), 503 (M₂H⁺), 525 (M₂Na⁺).

Anal. Found: C, 66.52; H, 8.54; N, 5.54. Calc for C₁₄H₂₁NO₃: C, 66.91; H, 8.42; N, 5.57.

### Preparation 20

### N-[(1,4-Dichloro-7-isoquinolinyl)sulphonyl]-N-(2-methoxybenzyl)glycine t-butyl ester

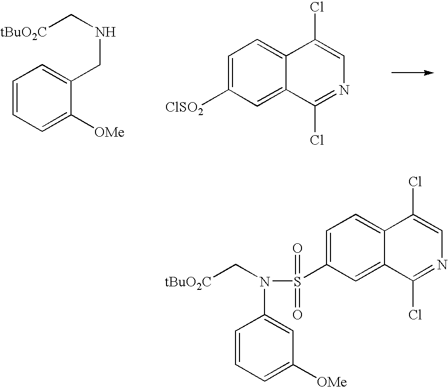

1,4-Dichloro-7-isoquinolinesulphonyl chloride (500 mg, 1.69 mmol) was added to a stirred solution of N-(2-methoxybenzyl)glycine t-butyl ester (508 mg, 2.02 mmol) in CH₂Cl₂ (30 mL) containing NEt₃ (0.35 mL, 2.5 mmol) under N₂ and the mixture was stirred at room temperature for 21 h. The mixture was diluted with CH₂Cl₂ and washed with dilute HCl (2 M), saturated aqueous NaHCO₃, brine, dried (Na₂SO₄) then and evaporated in vacuo. The residue was purified by column chromatography upon silica gel using hexane-EtOAc (95:5 to 90:10) as eluant and then triturated with hexane-i-Pr₂O to give N-[(1,4-dichloro-7-isoquinolinyl)sulphonyl]-N-(2-methoxybenzyl)glycine 1-butyl ester (501 mg, 1.02 mmol) as a yellow solid.

mp 106-108° C.

¹H (CDCl₃, 400 MHz) δ 1.3 (9H, s), 3.7 (3H, s), 4.0 (2H, s), 4.6 (2H, s), 6.8 (1H, d), 6.9 (1H, dd), 7.2 (1H, dd), 7.3 (1H, d), 8.2 (1H, d), 8.3 (1H, d), 8.45 (1H, s), 8.8 (1H, s) ppm.

LRMS 511, 513 (MH⁺), 528 (MNH₄⁺).

Anal. Found: C, 54.09; H, 4.78; N, 5.33. Calc for C₂₃H₂₄Cl₂N₂O₅S: C, 54.01; H, 4.73; N, 5.48.

### Preparation 21

### N-(3-Methoxybenzyl)glycine t-butyl ester

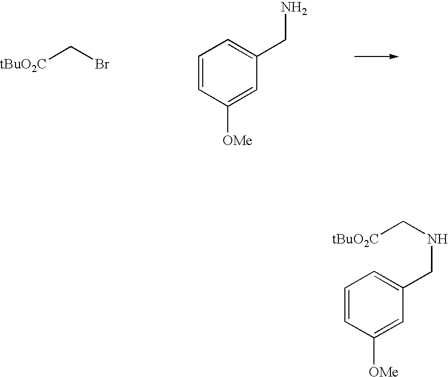

A solution of t-butyl bromoacetate (1.5 mL, 10.1 mmol) in CH₂Cl₂ (30 mL) was added dropwise to a stirred solution of 3-methoxybenzylamine (6.86 g, 50 mmol) in CH₂Cl₂ (20 mL) at 0° C., and the mixture was then warmed to room temperature and stirred for 1.5 h. Dilute HCl (30 mL, 1 M) was added and the mixture stirred for 15 min. The aqueous phase was extracted with CH₂Cl₂ and the combined organic extracts were washed with water, brine, saturated aqueous NaHCO₃, dried (MgSO₄) then and evaporated in vacuo. The residue was purified by column chromatography upon silica gel using CH₂Cl₂-MeOH (99:1 to 90:10) as eluant to give the required amine as a colourless oil. Treatment with a solution of HCl in ether (1 M) gave N-(3-methoxybenzyl)glycine t-butyl ester hydrochloride (0.83 g, 2.88 mmol) as a white solid.

mp 141-142° C.

¹H (CDCl₃, 300 MHz) δ 1.45 (9H, s), 3.5 (2H, s), 3.85 (3H, s), 4.35 (2H, s), 6.9 (1H, d), 7.1 (1H, d), 7.3 (1H, s), 7.3-7.35 (1H, m), 10.3 (2H, br s) ppm.

LRMS 252 (MH⁺), 503 (M₂H⁺).

Anal. Found: C, 58.37; H, 7.75; N, 4.83. Calc for C₁₄H₂₁NO₃.HCl: C, 58.43; H, 7.71; N, 7.71; N, 4.87.

### Preparation 22

### N-[(1,4-Dichloro-7-isoquinolinyl)sulphonyl]-N-(3-methoxybenzyl)glycine t-butyl ester

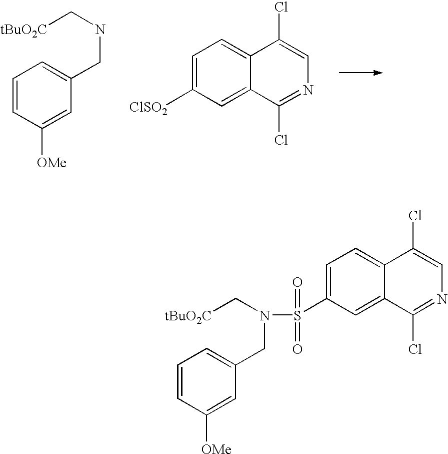

NEt₃ (0.59 mL, 4.24 mmol) and then 1,4-dichloro-7-isoquinolinesulphonyl chloride (500 mg, 1.68 mmol) were added to a stirred solution of N-(3-methoxybenzyl)glycine t-butyl ester hydrochloride (582 mg, 2.02 mmol) in CH₂Cl₂ (25 mL) under N₂ and the mixture was stirred at room temperature for 18 h. The mixture was diluted with CH₂Cl₂ (25 mL), washed with dilute HCl (×2, 1 M), saturated aqueous NaHCO₃, brine, dried (MgSO₄) and evaporated in vacuo. The residue was extracted with i-Pr₂O which gave a precipitate on standing. The white solid was collected by filtration and dried to give N-[(1,4-dichloro-7-isoquinolinyl)sulphonyl]-N-(3-methoxybenzyl)glycine t-butyl ester (262 mg, 0.51 mmol). A second batch (165 mg, 0.32 mmol) was obtained by evaporation of the mother liquors and purification of the residue by column chromatography upon silica gel using hexane-EtOAc (80:20).

mp 129-131° C.

¹H (CDCl₃, 300 MHz) δ 1.3 (9H, s), 3.75 (3H, s), 3.9 (2H, s), 4.55 (2H, s), 6.8-6.9 (2H, m), 6.85 (1H, s), 7.25 (1H, m), 8.3 (1H, d), 8.4 (1H, d), 8.5 (1H, s), 8.9 (1H, s) ppm.

LRMS 511 (MH⁺), 528 (MNH₄⁺).

Anal. Found: C, 54.03; H, 4.79; N, 5.34. Calc for C₂₃H₂₄Cl₂N₂O₅S: C, 54.01; H, 4.73; N, 5.48.

### Preparation 23

### N-[(1,4-Dichloro-7-isoquinolinyl)sulphonyl]-N-(3-chlorobenzyl)glycine t-butyl ester

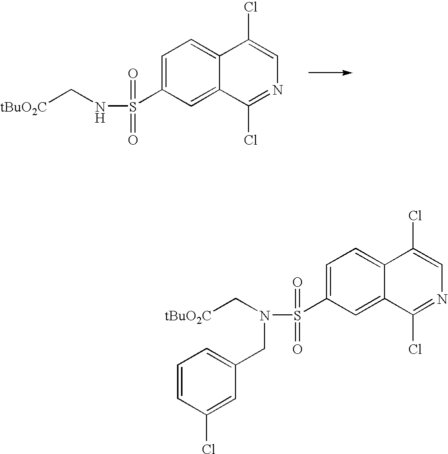

3-Chlorobenzyl chloride (0.063 mL, 0.50 mmol) was added to a stirred solution of N-[(1,4-dichloro-7-isoquinolinyl)sulphonyl]glycine t-butyl ester (195.5 mg, 0.50 mmol) in DMF (5 mL) containing K₂CO₃ (83 mg, 0.60 mmol) and the mixture was stirred at room temperature for 18 h. The mixture was diluted with water (50 mL), extracted with Et₂O (3×30 mL) and with EtOAc (3×30 mL), and the combined organic extracts were then washed with water, brine, dried (Na₂SO₄) and evaporated in vacuo. The solid was triturated with hexanes, collected by filtration and dried to give N-[(1,4-dichloro-7-isoquinolinyl)sulphonyl]-N-(3-chlorobenzyl)glycine t-butyl ester (212 mg, 0.41 mmol) as a pale yellow solid.

mp 141-143° C.

¹H (CDCl₃, 400 MHz) δ 1.3 (9H, s), 3.95 (2H, d), 4.5 (2H, s), 7.15-7.3 (4H, m), 8.25 (1H, d), 8.35 (1H, d), 8.5 (1H, s), 8.85 (1H, s) ppm.

LRMS 515, 517 (MH⁺), 532, 534 (MNH₄⁺).

Anal. Found: C, 51.14; H, 4.14; N, 5.31. Calc for C₂₂H₂₁Cl₃N₂O₄S: C, 51.22; H, 4.10; N, 5.43.

### Preparation 24

### N-(4-Methoxybenzyl)glycine t-butyl ester

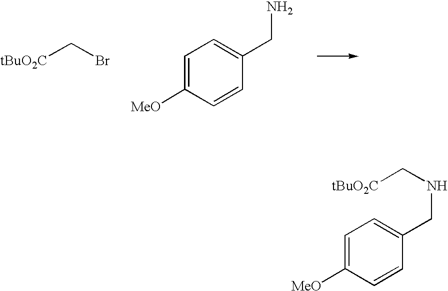

A solution of t-butyl bromoacetate (1.5 mL, 10.2 mmol) in CH₂Cl₂ (30 mL) was added dropwise to a stirred solution of 4-methoxybenzylamine (6.89 g, 50.2 mmol) in CH₂Cl₂ (70 mL) at 0° C., and the mixture was then warmed to room temperature and stirred for 1 h. Dilute HCl (30 mL, 1 M) was added and the mixture stirred for 10 min. The aqueous phase was extracted with CH₂Cl₂ and the combined organic extracts were washed with saturated aqueous NaHCO₃, brine, dried (Na₂SO₄) then and evaporated in vacuo. The residue was purified by column chromatography upon silica gel using CH₂Cl₂-MeOH (99:1 to 90:10) as eluant to give the required amine as a colourless oil. Treatment with a solution of HCl in ether (1 M) followed by trituration with Et₂O gave N-(4-methoxybenzyl)glycine t-butyl ester hydrochloride (148 mg, 0.51 mmol) as an orange solid.

mp 133-134° C.

¹H (CDCl₃, 400 MHz) δ 1.45 (9H, s), 3.5 (2H, s), 3.8 (3H, s), 4.3 (2H, s), 6.9 (2H, d), 7.5 (2H, d), 10.2 (2H, br s) ppm.

LRMS 252 (MH⁺), 503 (M₂H⁺), 525 (M₂Na⁺).

Anal. Found: C, 58.08; H, 7.71; N, 4.80. Calc for C₁₄H₂₁NO₃.HCl: C, 58.42; H, 7.71; N, 4.87.

### Preparation 25

### N-[(1,4-Dichloro-7-isoquinolinyl)sulphonyl]-N-(4-methoxybenzyl)glycine t-butyl ester

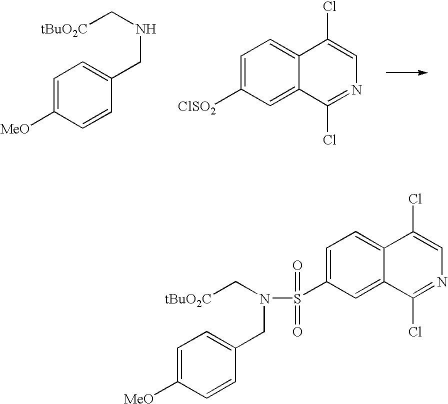

NEt₃ (0.25 mL, 1.78 mmol) and then 1,4-dichloro-7-isoquinolinesulphonyl chloride (210 mg, 0.71 mmol) were added to a stirred solution of N-(4-methoxybenzyl)glycine t-butyl ester hydrochloride (245 mg, 0.85 mmol) in CH₂Cl₂ (20 mL) under N₂ and the mixture was stirred at room temperature for 18 h. The mixture was diluted with CH₂Cl₂, washed with dilute HCl (2 M), saturated aqueous NaHCO₃, brine, dried (Na₂SO₄) and evaporated in vacuo. The residue was purified by column chromatography upon silica gel using hexane-EtOAc (95:5 to 90:10) as eluant and then triturated with hexane-i-Pr₂O to give N-[(1,4-dichloro-7-isoquinolinyl)sulphonyl]-N-(4-methoxybenzyl)glycine t-butyl ester (160 mg, 0.31 mmol) as a white solid.

mp 117-118° C.

¹H (CDCl₃, 300 MHz) δ 1.3 (9H, s), 3.8 (3H, s), 3.9 (2H, s), 4.5 (2H, s), 6.85 (2H, d), 7.2 (2H, d), 8.3 (1H, d), 8.35 (1H, d), 8.5 (1H, s), 8.9 (1H, s) ppm.

LRMS 511 (MH⁺), 528 (MNH₄⁺).

Anal. Found: C, 53.90; H, 4.59; N, 5.34. Calc for C₂₃H₂₄Cl₂N₂O₅S: C, 54.01; H, 4.73; N, 5.48.

### Preparation 26

### N-[(1,4-Dichloro-7-isoquinolinyl)sulphonyl]-N-(2-pyridylmethyl)glycine t-butyl ester

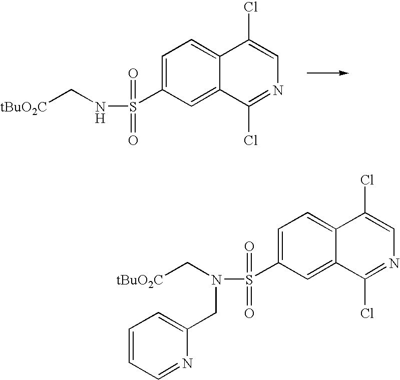

2-(Chloromethyl)pyridine hydrochloride (246 mg, 1.5 mmol) was added to a stirred solution of N-[(1,4-dichloro-7-isoquinolinyl)sulphonyl]glycine t-butyl ester (391 mg, 1.0 mmol) and anhydrous K₂CO₃ (415 mg, 3.0 mmol) in DMF (5 mL) under N₂ at 23° C. and the mixture was stirred for 18 h. The cooled mixture was azeotroped with xylene, diluted with EtOAc, washed with water, and the organic extracts were then dried (MgSO₄) and evaporated in vacuo. The residue was purified by column chromatography upon silica gel using pentane-EtOAc (100:0 to 50:50) as eluant to give N-[(1,4-dichloro-7-isoquinolinyl)sulphonyl]-N-(2-pyridylmethyl)glycine t-butyl ester (400 mg, 0.83 mmol) as a white solid.

¹H (CDCl₃, 400 MHz) δ 1.3 (9H, s), 4.1 (2H, s), 4.7 (2H, s), 7.1 (1H, m), 7.5 (1H, d), 7.7 (1H, dd), 8.25 (1H, d), 8.35 (1H, d), 8.45 (1H, m), 8.5 (1H, s), 8.9 (1H, s) ppm.

LRMS 482, 484 (MH⁺).

### Preparation 27

### N-[(1,4-Dichloro-7-isoquinolinyl)sulphonyl]-N-(3-pyridylmethyl)glycine t-butyl ester

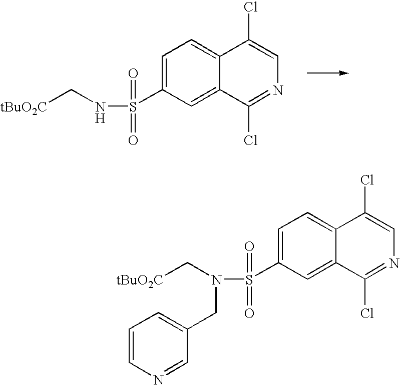

3-(Chloromethyl)pyridine hydrochloride (246 mg, 1.5 mmol) was added to a stirred solution of N-[(1,4-dichloro-7-isoquinolinyl)sulphonyl]glycine t-butyl ester (391 mg, 1.0 mmol) and anhydrous K₂CO₃ (416 mg, 3.0 mmol) in DMF (5 mL) under N₂ at 23° C. and the mixture was stirred for 18 h. The cooled mixture was azeotroped with xylene, diluted with EtOAc, washed with water, and the organic extracts were then dried (MgSO₄) and evaporated in vacuo. The residue was purified by column chromatography upon silica gel using pentane-EtOAc (100:0 to 50:50) as eluant to give N-[(1,4-dichloro-7-isoquinolinyl)sulphonyl]-N-(3-pyridylmethyl)glycine t-butyl ester (400 mg, 0.83 mmol) as a white solid.

¹H (CDCl₃, 400 MHz) δ 1.3 (9H, s), 4.1 (2H, d), 4.7 (2H, s), 7.1 (1H, m), 7.5 (1H, d), 7.7 (1H, dd), 8.25(1H, d), 8.35 (1H, d), 8.45(1H, m), 8.5(1H, s), 8.9 (1H, s) ppm.

LRMS 482, 484 (MH⁺).

### Preparation 28

### N-[(1,4-Dichloro-7-isoquinolinyl)sulphonyl]-N-(4-pyridylmethyl)glycine t-butyl ester

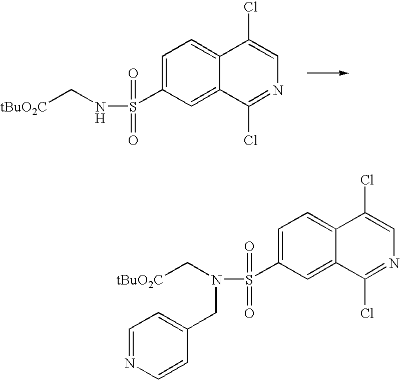

4-(Chloromethyl)pyridine hydrochloride (246 mg, 1.5 mmol) was added to a stirred solution of N-[(1,4-dichloro-7-isoquinolinyl)sulphonyl]glycine t-butyl ester (391 mg, 1.0 mmol) and anhydrous K₂CO₃ (416 mg, 3.0 mmol) in DMF (5 mL) under N₂ at 23° C. and the mixture was stirred for 18 h. The cooled mixture was azeotroped with xylene, diluted with EtOAc, washed with water, and the organic extracts were then dried (MgSO₄) and evaporated in vacuo. The residue was purified by column chromatography upon silica gel using pentane-EtOAc (100:0 to 50:50) as eluant to give N-[(1,4-dichloro-7-isoquinolinyl)sulphonyl]-N-(4-pyridylmethyl)glycine t-butyl ester (397 mg, 0.82 mmol) as a white solid.

¹H (CDCl₃, 400 MHz) δ 1.3 (9H, s), 4.0 (2H, d), 4.6 (2H, s), 7.3 (2H, d), 8.25 (1H, dd), 8.4 (1H, d), 8.5 (1H, s), 8.6 (2H, d), 8.9 (1H, d) ppm.

LRMS 482, 484 (MH⁺).

### Preparation 29

### N-[(1R)-1-Phenylethyl)]glycine t-butyl ester

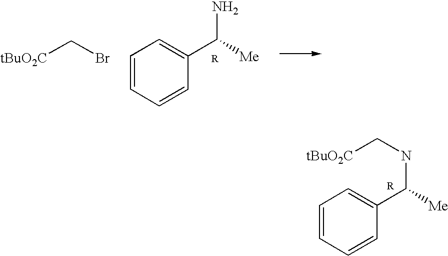

A solution of t-butyl bromoacetate (5.0 g, 25.6 mmol) in CH₂Cl₂ (5 mL) was added dropwise to a stirred solution of (+)-(R)-α-methylbenzylamine (4.65 g, 38.5 mmol) in CH₂Cl₂ (40 mL) at 0° C., and the mixture was then warmed to room temperature and stirred for 18 h. The mixture was diluted with CH₂Cl₂, washed with water, with dilute HCl (1 M) and then dried (MgSO₄). The solvents were evaporated in vacuo to give N-[(1R)-1-phenylethyl)]glycine t-butyl ester (3.15 g, 13.4 mmol) as a white powder.

mp 193-197° C.

¹H (CDCl₃, 300 MHz) δ 1.4 (9H, s), 1.95 (3H, d), 3.3 (1H, d), 3.6 (1H, d), 4.6 (1H, q), 5.3 (1H, s), 7.3-7.45 (3H, m), 7.5-7.65 (2H, m).

LRMS 236 (MH⁺).

### Preparation 30

### N-[(1,4-Dichloro-7-isoquinolinyl)sulphonyl]-N-[(1R)-1-phenylethyl)]glycine t-butyl ester

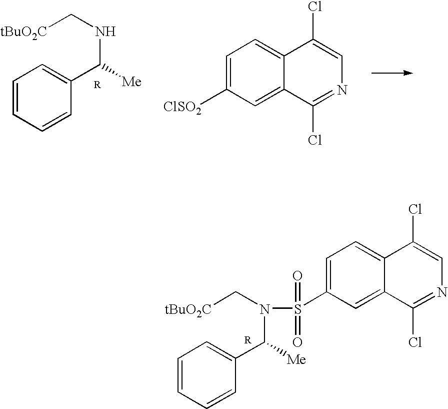

A mixture of NEt₃ (0.59 mL, 4.21 mmol), 1,4-dichloro-7-isoquinolinesulphonyl chloride (500 mg, 1.69 mmol) and N-[(1R)-1-phenylethyl)]glycine t-butyl ester (476 mg, 2.02 mmol) in CH₂Cl₂ (8 mL) were stirred under N₂ at room temperature for 18 h. The mixture was diluted with CH₂Cl₂ (50 mL), washed with dilute HCl (2 M), saturated aqueous NaHCO₃, brine, dried (Na₂SO₄) and evaporated in vacuo. The residue was purified by column chromatography upon silica gel using pentane-EtOAc (90:10) as eluant to give N-[(1,4-dichloro-7-isoquinolinyl)sulphonyl]-N-[(1R)-1-phenylethyl)]glycine t-butyl ester (490 mg, 0.99 mmol) as a colourless oil.

¹H (CDCl₃, 300 MHz) δ 1.3 (9H, s), 1.4 (3H, d), 3.9 (1H, d), 4.1 (1H, d), 5.15 (1H, q), 7.1-7.25 (5H, m), 8.4 (1H, d), 8.5 (1H, d), 8.65 (1H, s), 8.7 (1H, d) ppm.

LRMS 495 (MH⁺), 512 (MNH₄⁺).

### Preparation 31

### N-[(1S)-1-Phenylethyl)]glycine t-butyl ester

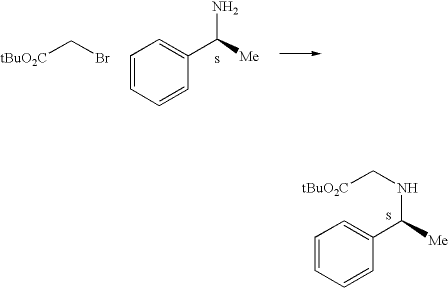

A solution of t-butyl bromoacetate (5.0 g, 25.6 mmol) in CH₂Cl₂ (5 mL) was added dropwise to a stirred solution of (−)-(S)-α-methylbenzylamine (4.65 g, 38.5 mmol) in CH₂Cl₂ (40 mL) at 0° C., and the mixture was then warmed to room temperature and stirred for 18 h. The mixture was diluted with CH₂Cl₂, washed with water, with dilute HCl (1 M) and then dried (MgSO₄). The solvents were evaporated in vacuo to give N-[(1S)-1-phenylethyl)]glycine t-butyl ester (2.02 g, 8.6 mmol) as a white powder.

mp 197-202° C.

¹H (CDCl₃, 300 MHz) δ 1.4 (9H, s), 1.9 (3H, d), 3.3 (1H, d), 3.55 (1H, d), 4.5 (1H, q), 5.3 (1H, s), 7.3-7.45 (3H, m), 7.5-7.6 (2H, m) ppm.

LRMS 236 (MH⁺).

### Preparation 32

### N-[(1,4-Dichloro-7-isoquinolinyl)sulphonyl]-N-[(1S)-1-phenylethyl)]glycine t-butyl ester

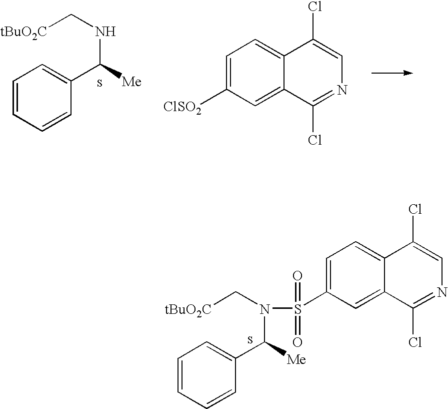

A mixture of NEt₃ (0.59 mL, 4.21 mmol), 1,4-dichloro-7-isoquinolinesulphonyl chloride (500 mg, 1.69 mmol) and N-[(1S)-1-phenylethyl))glycine t-butyl ester (476 mg, 2.02 mmol) in CH₂Cl₂ (8 mL) were stirred under N₂ at room temperature for 24 h. The mixture was diluted with CH₂Cl₂ (50 mL), washed with dilute HCl (2 M), saturated aqueous NaHCO₃, brine, dried (Na₂SO₄) and evaporated in vacuo. The residue was purified by column chromatography upon silica gel using pentane-EtOAc (90:10) as eluant to give N-[(1,4-dichloro-7-isoquinolinyl)sulphonyl)-N-[(1S)-1-phenylethyl)]glycine t-butyl ester (420 mg, 0.85 mmol) as a colourless oil.

¹H (CDCl₃, 300 MHz) δ 1.3 (9H, s), 1.4 (3H, d), 3.9 (1H, d), 4.1 (1H, d), 5.15 (1H, q), 7.1-7.25 (5H, m), 8.4 (1H, d), 8.5 (1H, d), 8.65 (1H, s), 8.7 (1H, d) ppm.

LRMS 495 (MH⁺), 512 (MNH₄⁺).

### Preparation 33

### N-Benzyl-L-alanine t-butyl ester

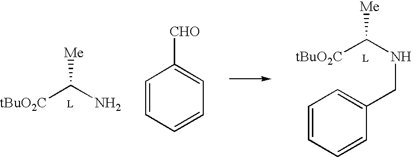

Benzaldehyde (2.69 mL, 26.4 mmol) was added to a stirred slurry of L-alanine t-butyl ester (4.0 g, 22.0 mmol) and NEt₃ (3.07 mL, 22.0 mmol) in CH₂Cl₂ (70 mL) at 23° C. and the mixture was stirred for 10 min. NaBH(OAc)₃ (6.44 g, 30.4 mmol) was added portionwise and the mixture stirred at 23° C. for 24 h. The mixture was washed with water, dried (MgSO₄) and the solvents were evaporated in vacuo. The residue was purified by column chromatography upon silica gel using CH₂Cl₂-MeOH (99:1 to 95:5) as eluant to give to give N-benzyl-L-alanine t-butyl ester (3.97 g, 16.9 mmol) as a colourless oil.

¹H (CDCl₃, 300 MHz) δ 1.3 (3H, d), 1.5 (9H, s), 2.1 (1H, s), 3.25 (1H, q), 3.7 (1H, d), 3.8 (1H, d), 7.2-7.4 (5H, m) ppm.

LRMS 236 (MH⁺), 258 (MNa⁺).

### Preparation 34

### N-Benzyl-N-[(1,4-dichloro-7-isoquinolinyl)sulphonyl]-L-alanine t-butyl ester

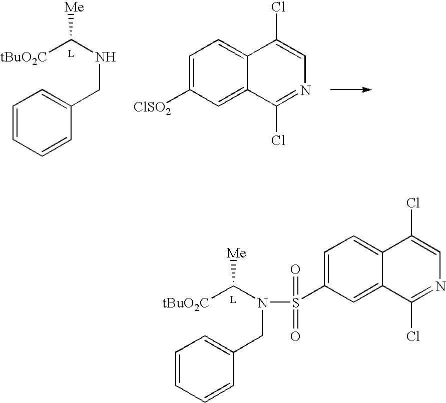

A solution of 1,4-dichloro-7-isoquinolinesulphonyl chloride (600 mg, 2.02 mmol) in CH₂Cl₂ (3 mL) was added to a stirred solution of N-benzyl-L-alanine t-butyl ester (571 mg, 2.43 mmol) and NEt₃ (0.70 mL, 5.06 mmol) in CH₂Cl₂ (3 mL) and the mixture was stirred at room temperature for 24 h. The mixture was diluted with CH₂Cl₂ (50 mL), washed with dilute HCl (2 M), saturated aqueous NaHCO₃, brine, dried (Na₂SO₄) and evaporated in vacuo. The residue was purified by column chromatography upon silica gel using pentane-EtOAc (95:5 to 85:15) as eluant to give N-benzyl-N-[(1,4-dichloro-7-isoquinolinyl)sulphonyl]-L-alanine t-butyl ester (470 mg, 0.95 mmol) as a colourless solid.

mp 92-96° C.

¹H (CDCl₃, 300 MHz) δ 1.3 (9H, s), 1.35 (3H, d), 4.4 (1H, d), 4.7 (1H, q), 4.8 (1H, d), 7.1-7.3 (3H, m), 7.3-7.4 (2H, m), 8.15 (1H, d), 8.3 (1H, d), 8.45 (1H, s), 8.7 (1H, s) ppm.

LRMS 495 (MH⁺).

### Preparation 35

### N-[(1,4-Dichloro-7-isoquinolinyl)sulphonyl]-L-alanine t-butyl ester

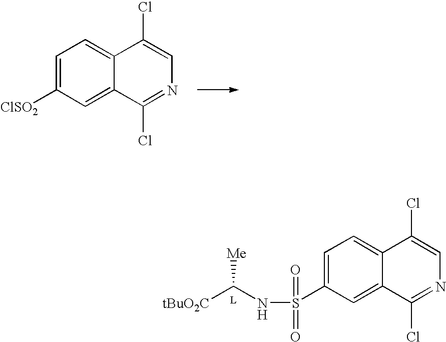

A solution of 1,4-dichloro-7-isoquinolinesulphonyl chloride (500 mg, 1.69 mmol) in CH₂Cl₂ (3 mL) was added to a stirred solution of L-alanine t-butyl ester (322 mg, 1.77 mmol) and NEt₃ (0.82 mL, 5.9 mmol) in CH₂Cl₂ (6 mL) and the mixture was stirred at 23° C. for 17 h. The mixture was diluted with CH₂Cl₂, washed with dilute HCl (2 M), saturated aqueous NaHCO₃, brine, dried (MgSO₄) and evaporated in vacuo. The residue was purified by column chromatography upon silica gel using pentane-EtOAc (90:10 to 50:50) as eluant to give N-[(1,4-dichloro-7-isoquinolinyl)sulphonyl)-L-alanine t-butyl ester (500 mg, 1.23 mmol) as a white powder.

mp 115-119° C.

¹H (CDCl₃, 300 MHz) δ 1.2 (9H, s), 1.4 (3H, d), 4.0 (1H, dq), 5.4 (1H, d), 8.25 (1H, d), 8.4 (1H, d), 8.5 (1H, s), 8.9 (1H, s) ppm.

LRMS 405 (MH⁺).

Anal. Found: C, 47.57; H, 4.39; N, 6.72. Calc for C₁₆H₁₈Cl₂N₂O₄S: C, 47.42; H, 4.48; N, 6.91.

### Preparation 36

### N-[(1,4-Dichloro-7-isoquinolinyl)sulphonyl]-D-alanine methyl ester

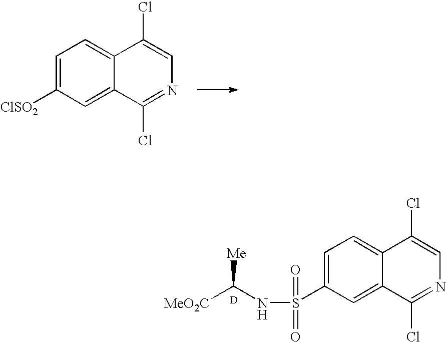

A solution of 1,4-dichloro-7-isoquinolinesulphonyl chloride (500 mg, 1.69 mmol) in CH₂Cl₂ (3 mL) was added to a stirred solution of D-alanine methyl ester (247 mg, 1.77 mmol) and NEt₃ (0.82 mL, 5.9 mmol) in CH₂Cl₂ (6 mL) and the mixture was stirred at 23° C. for 16 h. The mixture was diluted with CH₂Cl₂, washed with dilute HCl (2 M), saturated aqueous NaHCO₃, brine, dried (MgSO₄) and evaporated in vacuo. The residue was purified by column chromatography upon silica gel using pentane-EtOAc (90:10 to 50:50) as eluant to give N-[(1,4-dichloro-7-isoquinolinyl)sulphonyl]-D-alanine methyl ester (420 mg, 1.16 mmol) as a white powder.

mp 150-152° C.

¹H (CDCl₃, 300 MHz) δ 1.45 (3H, d), 3.55 (3H, s), 4.15 (1H, dq), 5.4 (1H, d), 8.2 (1H, d), 8.4 (1H, d), 8.5 (1H, s), 8.9 (1H, s) ppm.

LRMS 363, 365 (MH⁺).

Anal. Found: C, 42.97; H, 3.29; N, 7.42. Calc for C₁₃H₁₂Cl₂N₂O₄S: C, 42.99; H, 3.33; N, 7.71.

### Preparation 37

### N-[(1,4-Dichloro-7-isoquinolinyl)sulphonyl]-L-valine t-butyl ester

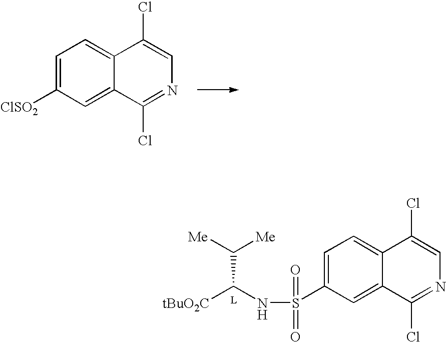

NEt₃ (0.59 mL, 4.2 mmol) was added to a stirred mixture of 1,4-dichloro-7-isoquinolinesulphonyl chloride (500 mg, 1.69 mmol) and L-valine t-butyl ester (354 mg, 1.69 mmol) and in CH₂Cl₂ (25 mL) and the mixture was stirred at 23° C. for 3 d. The mixture was washed with dilute HCl (2×20 mL, 1 M), saturated aqueous NaHCO₃, brine, dried (MgSO₄) and evaporated in vacuo. The residue was extracted with hexane, which crystallised on standing, to give N-[(1,4-dichloro-7-isoquinolinyl)sulphonyl]-L-valine t-butyl ester (463 mg, 1.07 mmol) as a white solid.

mp 127-129° C.

¹H (CDCl₃, 300 MHz) δ 0.9 (3H, d), 1.0 (3H, d), 1.1 (9H, s), 2.0-2.2 (1H, m), 3.8 (1H, dd), 5.25 (1H, d), 8.2 (1H, d), 8.35 (1H, d), 8.5 (1H, s), 8.9 (1H, s) ppm.

LRMS 433, 435 (MH⁺), 450, 452 (MNH₄⁺).

Anal. Found: C, 49.86; H, 5.13; N, 6.40. Calc for C₁₈H₂₂Cl₂N₂O₄S: C, 49.89; H, 5.18; N, 6.46.

### Preparation 38

### N-[(1,4-Dichloro-7-isoquinolinyl)sulphonyl]-D-valine t-butyl ester

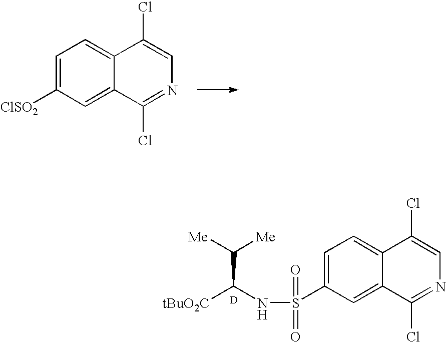

D-Valine t-butyl ester has been prepared previously, see: Shepel, E. N.; Iodanov, S.; Ryabova, I. D.; Miroshnikov, A. I.; Ivanov, V. T.; Ovchinnikov, Yu A. Bioorg. Khim. 1972, 2, 581-593.

D-Valine t-butyl ester (354 mg, 1.69 mmol) and then NEt₃ (0.59 mL, 4.2 mmol) were added to a stirred solution of 1,4-dichloro-7-isoquinolinesulphonyl chloride (500 mg, 1.69 mmol) and in CH₂Cl₂ (20 mL and the mixture was stirred at 23° C. for 16 h. The mixture was diluted with CH₂Cl₂ (50 mL), washed with saturated aqueous NaHCO₃, water, aqueous citric acid (1 M), water, brine, dried (MgSO₄) and evaporated in vacuo. The residue was dissolved in i-Pr₂O and hexane was added which gave a precipitate. The solvents were evaporated in vacuo and the solid was triturated with hexane to give N-[(1,4-dichloro-7-isoquinolinyl)sulphonyl]-D-valine t-butyl ester (532 mg, 1.22 mmol) as a white solid. An analytical sample was obtained by recrystallisation from hexane.

mp 117-119° C.

¹H (CDCl₃, 400 MHz) δ 0.9 (3H, d), 1.0 (3H, d), 1.1 (9H, s), 2.0-2.2 (1H, m), 3.8 (1H, dd), 5.3 (1H, d), 8.2 (1H, d), 8.35 (1H, d), 8.5 (1H, s), 8.9 (1H, s) ppm.

LRMS 433, 435 (MH⁺).

Anal. Found: C, 49.99; H, 5.28; N, 6.34. Calc for C₁₈H₂₂Cl₂N₂O₄S: C, 49.89; H, 5.12; N, 6.46.

### Preparation 39

### N-[(1,4-Dichloro-7-isoquinolinyl)sulphonyl]-D-tert-leucine t-butyl ester

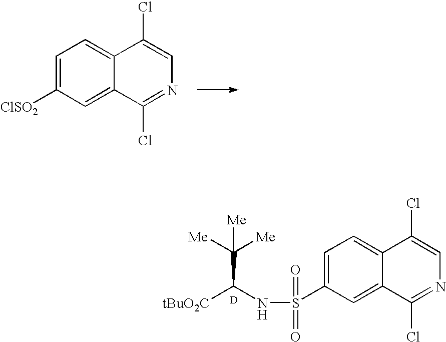

A mixture of D-tert-leucine t-butyl ester hydrochloride (250 mg, 1.12 mmol), NEt₃ (0.40 mL, 2.87 mmol) and 1,4-dichloro-7-isoquinolinesulphonyl chloride (330 mg, 1.11 mmol) in CH₂Cl₂ (20 mL) was stirred at 23° C. for 16 h. The mixture was diluted with CH₂Cl₂ (50 mL), washed with water, aqueous citric acid (1 M), water, saturated aqueous NaHCO₃, brine, dried (MgSO₄) and evaporated in vacuo. The residue was purified by column chromatography upon silica gel using hexane-EtOAc (90:10) as eluant to give N-[(1,4-dichloro-7-isoquinolinyl)sulphonyl]-D-tert-leucine t-butyl ester (250 mg, 0.56 mmol) as a white foam.

mp 140-142° C.

¹H (CDCl₃, 400 MHz) δ 1.0 (9H, s), 1.05 (9H, s), 3.6 (1H, d), 5.35 (1H, d), 8.2 (1H, d), 8.35 (1H, d), 8.45 (1H, s), 8.85 (1H, s).

LRMS 447, 449, 451 (MH⁺).

Anal. Found: C, 51.03; H, 5.41; N, 6.13. Calc for C₁₉H₂₄Cl₂N₂O₄S: C, 51.01; H, 5.41; N, 6.26.

### Preparation 40

### N-[(1,4-Dichloro-7-isoquinolinyl)sulphonyl]-L-phenylalanine t-butyl ester

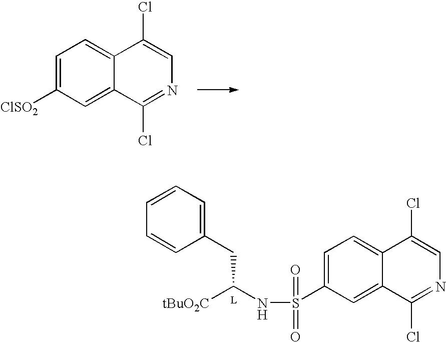

A mixture of L-phenylalanine t-butyl ester (352 mg, 1.37 mmol), NEt₃ (0.41 mL, 2.97 mmol) and 1,4-dichloro-7-isoquinolinesulphonyl chloride (399 mg, 1.35 mmol) in CH₂Cl₂ (10 mL) was stirred at 23° C. for 20 h. The solvents were evaporated in vacuo and the residue suspended in EtOAc. This solution was washed with water, brine, dried (MgSO₄) and evaporated in vacuo. The residue was purified by column chromatography upon silica gel using pentane-EtOAc (90:10 to 70:30) as eluant to give N-[(1,4-dichloro-7-isoquinolinyl)sulphonyl]-L-phenylalanine t-butyl ester (450 mg, 0.94 mmol) as a white crystallised foam.

¹H (CDCl₃, 300 MHz) δ 1.2 (9H, s), 2.95 (1H, dd), 3.1 (1H, dd), 4.1 (1H, m), 5.3 (1H, d), 7.0-7.2 (5H, m), 8.1 (1H, d), 8.25 (1H, d), 8.5 (1H, s), 8.75 (1H, d) ppm.

LRMS 481 (MH⁺), 498 (MNH₄⁺).

### Preparation 41

### N-(Benzyloxycarbonyl)-O-methyl-D-serine t-butyl ester

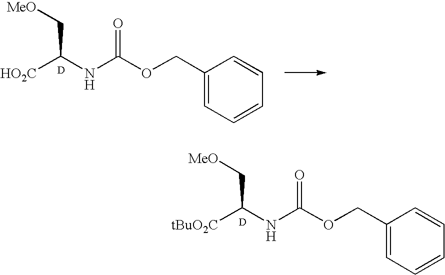

Condensed isobutylene gas (35 mL) was added to a solution of N-(benzyloxycarbonyl)-O-methyl-D-serine dicyclohexlamine salt (2.5 g, 5.76 mmol) in CH₂Cl₂ (35 mL) at −78° C. in a steel bomb. Conc. H₂SO₄ (0.5 mL) was added, the vessel was sealed and the mixture allowed to warm to 23° C. [CAUTION: Pressure]. The mixture was stirred at 23° C. for 6 d, the vessel was vented and excess isobutylene was allowed to evaporate. The mixture then poured into aqueous NaHCO₃ (30 mL, 10%), extracted with CH₂Cl₂ (3×30 mL), and the combined organic extracts were dried (Na₂SO₄) and evaporated in vacuo. The residue was purified by column chromatography upon silica gel using hexane-EtOAc (80:20) as eluant to give N-(benzyloxycarbonyl)-O-methyl-D-serine t-butyl ester (1.2 g, 3.88 mmol) as a colorless oil.

¹H (CDCl₃, 400 MHz) δ 1.45 (9H, s), 3.35 (3H, s), 3.6 (1H, dd), 3.75 (1H, dd), 4.35 (1H, br d), 5.1 (2H, s), 5.6 (1H, br d), 8.4-8.9 (5H, m) ppm.

LRMS 310 (MH⁺), 327 (MNH₄⁺).

### Preparation 42

### O-Methyl-D-serine t-butyl ester

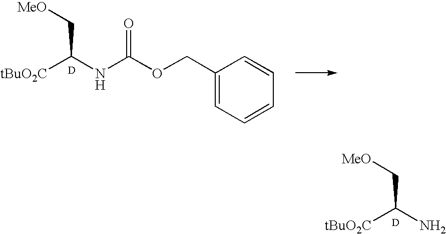

A solution of N-(benzyloxycarbonyl)-O-methyl-D-serine t-butyl ester (1.15 g, 3.72 mmol) in MeOH (20 mL) was hydrogenated over 10% Pd/C (150 mg) under an atmosphere of H₂ (15 psi) at 23° C. for 18 h. The mixture was filtered and the filtrate evaporated in vacuo. The residue was dissolved in Et₂O, a solution of HCl in Et₂O (1 M) was added, the solvents were evaporated in vacuo to give a white solid and this material was triturated with hexane to give O-methyl-D-serine t-butyl ester hydrochloride (0.62 g, 2.90 mmol).

mp 167-169° C. (dec).

¹H (CDCl₃, 400 MHz) δ 1.5 (9H, s), 1.8-2.2 (1H, br s), 3.4 (3H, s), 3.9 (1H, dd), 4.0 (1H, dd), 4.2 (1H, t), 8.4-8.9 (3H, br s) ppm.

LRMS 176 (MH⁺).

Anal. Found: C, 45.26; H, 8.59; N, 6.39. Calc for C₈H₁₇NO₃.HCl: C, 45.39; H, 8.57; N, 6.62.

### Preparation 43

### N-[(1,4-Dichloro-7-isoquinolinyl)sulphonyl]-O-methyl-D-serine t-butyl ester

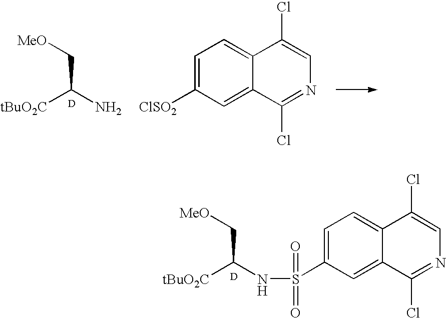

A mixture of O-methyl-D-serine t-butyl ester hydrochloride (300 mg, 1.42 mmol), NEt₃ (0.50 mL, 3.6 mmol) and 1,4-dichloro-7-isoquinolinesulphonyl chloride (420 mg, 1.42 mmol) in CH₂Cl₂ (20 mL) was stirred at 23° C. for 3 d. The mixture was diluted with CH₂Cl₂ (30 mL), washed with water, aqueous citric acid (1 M), water, saturated aqueous NaHCO₃, brine, dried (MgSO₄) and evaporated in vacuo. The residue was purified by column chromatography upon silica gel using hexane-EtOAc (80:20) as eluant to give N-[(1,4-dichloro-7-isoquinolinyl)sulphonyl]-O-methyl-D-serine t-butyl ester (356 mg, 0.82 mmol) as a white solid.

mp135-137° C.

¹H (CDCl₃, 400 MHz) δ 1.25 (9H, s), 3.3 (3H, s), 3.6 (1H, dd), 3.7 (1H, dd), 4.1 (1H, br s), 5.6 (1H, br d), 8.25 (1H, d), 8.35 (1H, d), 8.5 (1H, s), 8.9 (1H, s) ppm.

LRMS 435, 437 (MH⁺), 452, 454 (MNH₄₊).

Anal. Found: C, 47.04; H, 4.62; N, 6.42. Calc for C₁₇H₂₀Cl₂N₂O₅S: C, 46.90; H, 4.63; N, 6.44.

### Preparation 44

### N-[(1,4-Dichloro-7-isoquinolinyl)sulphonyl]-D-aspartic acid di-t-butyl ester

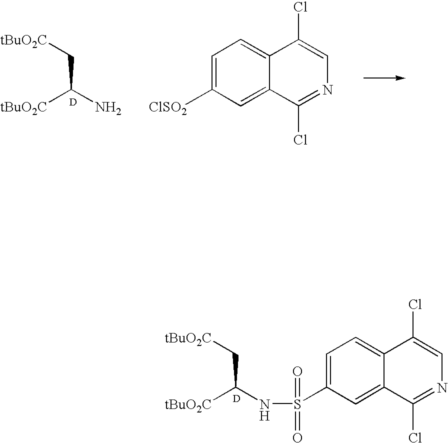

A mixture of D-aspartic acid di-t-butyl ester (462 mg, 1.64 mmol), NEt₃ (0.50 mL, 3.6 mmol) and 1,4-dichloro-7-isoquinolinesulphonyl chloride (400 mg, 1.35 mmol) in CH₂Cl₂ (30 mL) was stirred at 23° C. for 18 h. The mixture was diluted with CH₂Cl₂ (30 mL), washed with dilute HCl (2 M), saturated aqueous NaHCO₃, brine, dried (MgSO₄) and evaporated in vacuo to give N-[(1,4-dichloro-7-isoquinolinyl)sulphonyl)-D-aspartic acid di-t-butyl ester (520 mg, 1.03 mmol) as a white solid.

mp 106-110° C.

¹H (CDCl₃, 400 MHz) δ 1.2 (9H, s), 1.4 (9H, s), 2.7-2.8 (1H, dd), 2.8-2.9 (1H, dd), 4.15 (1H, m), 8.2 (1H, d), 8.4 (1H, d), 8.5 (1H, s), 8.9 (1H, s) ppm.

LRMS 507 (MH⁺).

### Preparation 45

### N-[(1,4-Dichloro-7-isoquinolinyl)sulphonyl]-L-proline t-butyl ester

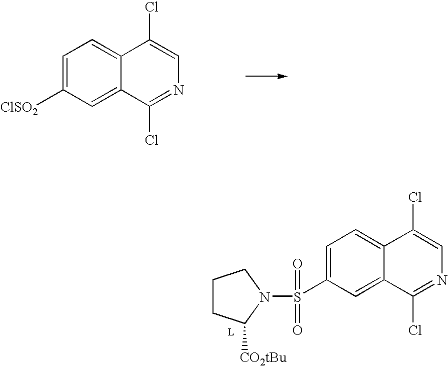

A mixture of L-proline t-butyl ester hydrochloride (335 mg, 1.61 mmol), NEt₃ (0.53 mL, 3.78 mmol) and 1,4-dichloro-7-isoquinolinesulphonyl chloride (449 mg, 1.51 mmol) in CH₂Cl₂ (10 mL) was stirred at 23° C. for 20 h. The solvents were evaporated in vacuo and the residue suspended in EtOAc. This solution was washed with water, brine, dried (MgSO₄) and evaporated in vacuo. The residue was purified by column chromatography upon silica gel using pentane-EtOAc (90:10 to 70:30) as eluant to give N-[(1,4-dichloro-7-isoquinolinyl)sulphonyl]-L-proline t-butyl ester (543 mg, 1.26 mmol) as a white solid.

¹H (CDCl₃, 300 MHz) δ 1.45 (9H, s), 1.8-2.1 (3H, m), 2.1-2.3 (1H, m), 3.4-3.6 (2H, m), 4.4 (1H, dd), 8.3 (1H, d), 8.4 (1H, d), 8.5 (1H, s), 8.9 (1H, d) ppm.

LRMS 431 (MH⁺), 448, 450 (MNH₄⁺).

Anal. Found: C, 50.09; H, 4.62; N, 6.37. Calc for C₁₈H₂₀Cl₂N₂O₄S: C, 50.12; H, 4.67; N, 6.49.

### Preparation 46

### N-[(1,4-Dichloro-7-isoquinolinyl)sulphonyl]-D-proline t-butyl ester

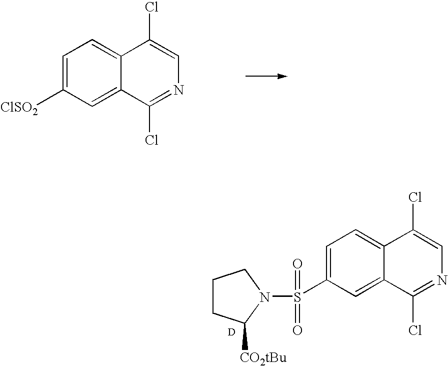

A mixture of D-proline t-butyl ester hydrochloride (340 mg, 1.64 mmol), NEt₃ (0.50 mL, 3.6 mmol) and 1,4-dichloro-7-isoquinolinesulphonyl chloride (400 mg, 1.35 mmol) in CH₂Cl₂ (30 mL) was stirred at 23° C. for 20 h. The mixture was diluted with CH₂Cl₂ (50 mL), washed with dilute HCl (2 M), saturated aqueous NaHCO₃, brine, dried (MgSO₄) and evaporated in vacuo to give N-[(1,4-dichloro-7-isoquinolinyl)sulphonyl]-D-proline t-butyl ester (550 mg, 1.28 mmol) as a white solid.

mp 80-82° C.

¹H (CDCl₃, 400 MHz) δ 1.4 (9H, s), 1.9-2.0 (3H, m), 2.2 (1H, m), 3.4-3.6 (2H, m), 4.4 (1H, m), 8.3 (1H, d), 8.4 (1H, d), 8.5 (1H, s), 8.9 (1H, s) ppm.

LRMS 431 (MH⁺),448 (MNH₄⁺).

Anal. Found: C, 49.76; H, 4.75; N, 6.39. Calc for C₁₈H₂₀Cl₂N₂O₄S: C, 50.12; H, 4.67; N, 6.49.

### Preparation 47

### 1,4-Dichloro-7-{[(2R)-(hydroxymethyl)-1-pyrrolidinyl]sulphonyl}isoquinoline

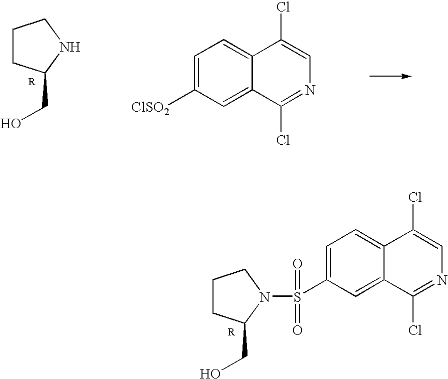

A mixture of (R)-2-pyrrolidinemethanol (1.1 mL, 11.0 mmol), NEt₃ (1.5 mL, 20 mmol) and 1,4-dichloro-7-isoquinolinesulphonyl chloride (3.0 g, 10 mmol) in CH₂Cl₂ (50 mL) was stirred at 23° C. for 30 min. The mixture was diluted with CH₂Cl₂ (50 mL), washed with aqueous citric acid (1 N), water, brine, dried (MgSO₄) and evaporated in vacuo to give 1,4-dichloro-7-{[(2R)-(hydroxymethyl)-1-pyrrolidinyl]sulphonyl}isoquinoline (4.0 g, 11 mmol) as a white solid.

mp 167.5-168.5° C.

¹H (CDCl₃, 400 MHz) δ 1.5-1.55 (1H, m), 1.6-2.0 (3H, m), 2.5 (1H, br t), 3.3-3.4 (1H, m), 3.5-3.6 (1H, m), 3.7-3.8 (3H, m), 8.25 (1H, d), 8.4 (1H, d), 8.5 (1H, s), 8.9 (1H, s) ppm.

LRMS 361, 363 (MH⁺), 378 (MNH₄⁺), 383 (MNa⁺).

Anal. Found: C, 46.65; H, 3.91; N, 7.61. Calc for C₁₄H₁₄Cl₂N₂O₃S: C, 46.55; H, 3.91; N, 7.75.

### Preparation 48

### Methyl 2-{[(1,4-dichloro-7-isoquinolinyl)sulphonyl]amino) isobutyrate

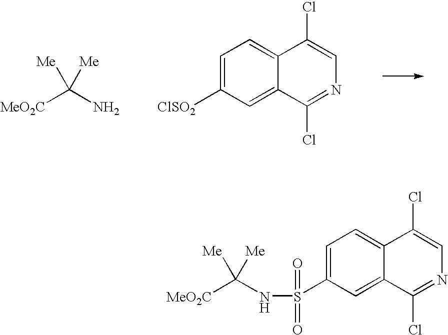

A mixture of methyl 2-aminoisobutyrate (310 mg, 2.02 mmol), NEt₃ (0.70 mL, 5.05 mmol) and 1,4-dichloro-7-isoquinolinesulphonyl chloride (500 mg, 1.69 mmol) in CH₂Cl₂ (30 mL) was stirred at 23° C. for 17 h. The mixture was diluted with CH₂Cl₂ (50 mL), washed with dilute HCl (2 M), saturated aqueous NaHCO₃, brine, dried (Na₂SO₄) and evaporated in vacuo The residue was purified by column chromatography upon silica gel using hexane-EtOAc (70:30) as eluant to give methyl 2-{[(1,4-dichloro-7-isoquinolinyl)sulphonyl]amino}isobutyrate (210 mg, 0.56 mmol) as a white solid.

mp 159.5-161° C.

¹H (CDCl₃, 400 MHz) δ 1.5 (6H, s), 3.7 (3H, s), 5.55 (1H, s), 8.25 (1H, d), 8.35 (1H, d), 8.5 (1H, s), 8.9 (1H, s) ppm.

LRMS 377 (MH⁺).

Anal. Found: C, 44.24; H, 3.72; N, 7.29. Calc for C₁₄H₁₄Cl₂N₂O₄S: C, 44.57; H, 3.74; N, 7.43.

### Preparation 49

### 2-{[(1,4-Dichloro-7-isoquinolinyl)sulphonyl]amino}-2-methylpropanamide

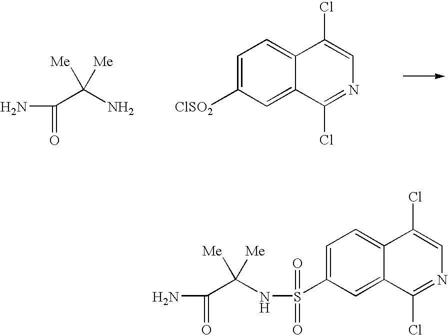

A mixture of 2-amino-2-methylpropanamide (200 mg, 1.96 mmol), NEt₃ (0.69 mL, 5.0 mmol) and 1,4-dichloro-7-isoquinolinesulphonyl chloride (580 mg, 1.96 mmol) in CH₂Cl₂ (20 mL) was stirred at 23° C. for 17 h. The mixture was diluted with CH₂Cl₂ (50 mL), washed with water, aqueous citric acid (1 N), water, brine, dried (MgSO₄) and evaporated in vacuo The residue was purified by column chromatography upon silica gel using CH₂Cl₂-MeOH-0.880NH₃ (90:10:1) as eluant to give 2-{[(1,4-dichloro-7-isoquinolinyl)sulphonyl]amino}-2-methylpropanamide (228 mg, 0.62 mmol) as a white solid.

mp 220-222° C.

¹H (d₄-MeOH, 400 MHz) δ 1.4 (6H, s), 3.3 (2H, s), 8.4 (1H, dd), 8.45 (1H, d), 8.55 (1H, d), 8.9 (1H, s).

LRMS 362, 364 (MH⁺), 379, 381 (MNH₄⁺).

Anal. Found: C, 42.81; H, 3.70; N, 11.15. Calc for C₁₃H₁₃Cl₂N₃O₃S.0.25H₂O: C, 42.58; H, 3.71; N, 11.46.

### Preparation 50

### Ethyl 1-aminocyclobutanecarboxylate

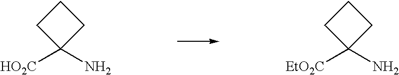

A solution 1-aminocyclobutanecarboxylic acid (500 mg, 4.34 mmol) in EtOH (10 mL) was saturated with HCl gas, and the mixture was stirred at 23° C. for 4 d. The solvents were evaporated in vacuo, azeotroping with PhMe and CH₂Cl₂, to give ethyl 1-aminocyclobutanecarboxylate hydrochloride (754 mg, 4.20 mmol) as an off-white solid.

¹H (DMSO-d₆, 300 MHz) δ 1.25 (3H, t), 1.9-2.1 (2H, m), 2.3-2.5 (4H, m), 4.2 (2H, q), 8.8 (2H, br s) ppm.

LRMS 287 (M₂H⁺).

### Preparation 51

### Ethyl 1-{([1,4-dichloro-7-isoquinolinyl)sulphonyl]amino}cyclobutanecarboxylate

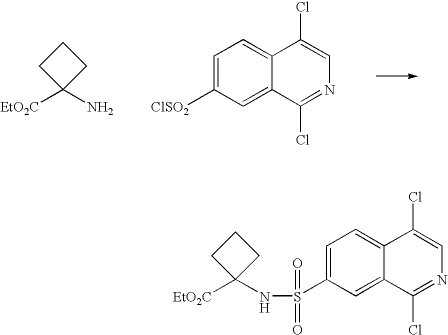

A mixture of ethyl 1-aminocyclobutanecarboxylate hydrochloride (382 mg, 2.12 mmol), NEt₃ (1.04 mL, 7.43 mmol) and 1,4-dichloro-7-isoquinolinesulphonyl chloride (630 mg, 2.12 mmol) in CH₂Cl₂ (8 mL) was stirred at 23° C. for 18 h. The mixture was diluted with CH₂Cl₂, washed with dilute HCl (2 M), saturated aqueous NaHCO₃, brine, dried (MgSO₄) and evaporated in vacuo. The residue was purified by column chromatography upon silica gel using pentane-EtOAc (90:10 to 80:20) as eluant to give ethyl 1-{[(1,4-dichloro-7-isoquinolinyl)sulphonyl]amino} cyclobutanecarboxylate (480 mg, 1.19 mmol) as a white powder.

mp 123-125° C.

¹H (CDCl₃, 300 MHz) δ 1.2 (3H, t), 1.9-2.1 (2H, m), 2.4-2.6 (4H, m), 4.0 (2H, q), 5.5 (1H, br s), 8.25 (1H, d), 8.4 (1H, d), 8.5 (1H, s), 8.9 (1H, s) ppm.

LRMS 403, 405 (MH⁺), 420 (MNH₄⁺).

### Preparation 52

### Cycloleucine Ethyl Ester

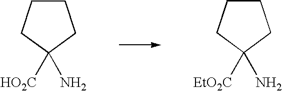

A solution of cycloleucine (8.94 g, 69.2 mmol) in EtOH (100 mL) was saturated with HCl gas, and the mixture was stirred at 23° C. for 2 d. The solvents were evaporated in vacuo, the residue was dissolved in water (200 mL) and the solution basified with solid NaHCO₃. The aqueous solution was extracted with EtOAc (3×100 mL) and the combined extracts were washed with brine, dried (MgSO₄) and evaporated in vacuo. The residue was dissolved in hexane-Et₂O (1:1) and a solution of HCl in Et₂O-dioxane (0.5 M, 1:1) was added which gave a precipitate. This off-white solid was collected by filtration and dried to give cycloleucine ethyl ester hydrochloride (6.57 g, 33.9 mmol).

¹H (d₆-DMSO, 400 MHz) δ 1.2 (3H, t), 1.6-1.8 (2H, m), 1.8-2.0 (4H, m), 2.05-2.15 (2H, m), 4.15 (2H, q), 8.6-8.7 (3H, br s) ppm.

### Preparation 53

### N-[(1,4-Dichloro-7-isoquinolinyl)sulphonyl]cycloleucine ethyl ester

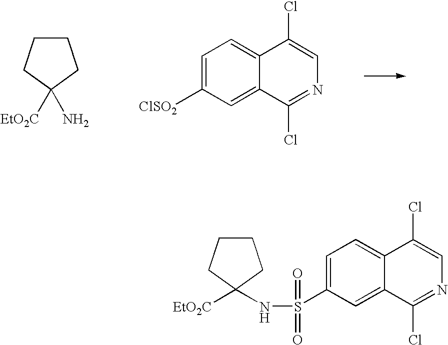

A mixture of cycloleucine ethyl ester hydrochloride (5.56 g, 28.7 mmol), NEt₃ (9.9 mL, 72 mmol) and 1,4-dichloro-7-isoquinolinesulphonyl chloride (7.10 g, 24.0 mmol) in CH₂Cl₂ (480 mL) was stirred at 23° C. for 3 d. The mixture was diluted with CH₂Cl₂, washed with dilute HCl (2 M), saturated aqueous NaHCO₃, brine, dried (Na₂SO₄) and evaporated in vacuo. The residue was purified by column chromatography upon silica gel using pentane-EtOAc (80:20 to 70:30) as eluant to give N-[(1,4-dichloro-7-isoquinolinyl)sulphonyl]cycloleucine ethyl ester (6.36 g, 15.2 mmol) as a white solid.

mp 127-129° C.

¹H (CDCl₃, 400 MHz) δ 1.2 (3H, t), 1.6-1.8 (4H, m), 1.9-2.0 (2H, m), 2.1-2.2 (2H, m), 4.1 (2H, q), 5.25 (1H, s), 8.25(1H, d), 8.35 (1H, d), 8.5 (1H, s), 8.9 (1H, s) ppm.

LRMS 417, 419 (MH⁺).

Anal. Found: C, 48.57; H. 4.35; N, 6.58. Calc for C₁₇H₁₈Cl₂N₃O₄S: C, 48.93; H, 4.35; N, 6.71.

### Preparation 54

### 1,4-Dichloro-N-[1-(hydroxymethyl)cyclopentyl]-7-isoquinolinesulphonamide

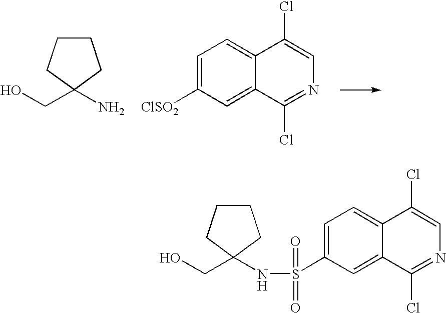

A mixture of 1-amino-1-cyclopentylmethanol (559 mg, 4.86 mmol), NEt₃ (0.85 mL, 6.0 mmol) and 1,4-dichloro-7-isoquinolinesulphonyl chloride (1.2 g, 4.05 mmol) in CH₂Cl₂ (80 mL) was stirred at 23° C. for 16 h. The mixture was diluted with CH₂Cl₂ (50 mL), washed with dilute HCl (2 M), saturated aqueous NaHCO₃, brine, dried (Na₂SO₄) and evaporated in vacuo. The residue was purified by column chromatography upon silica gel using CH₂Cl₂-MeOH-0.880NH₃ (95:5:0.5) as eluant, followed by trituration with Et₂O, to give to give 1,4-dichloro-N-[1-(hydroxymethyl)cyclopentyl]-7-isoquinolinesulphonamide (0.62 g, 1.65 mmol) as a white solid.

mp 148-150° C.

¹H (CDCl₃, 400 MHz) δ 1.5-1.6 (4H, m), 1.6-1.7 (2H, m), 1.7-1.8 (2H, m), 2.2 (1H, br t), 3.65 (2H, d), 5.1 (1H, s), 8.3 (1H, d), 8.35 (1H, d), 8.5 (1H, s), 8.9 (1H, s) ppm.

LRMS 375 (MH⁺).

### Preparation 55

### N-[(1,4-Dichloro-7-isoquinolinyl)sulphonyl]-N-[2-(dimethylamino)ethyl]cycloleucine ethyl ester

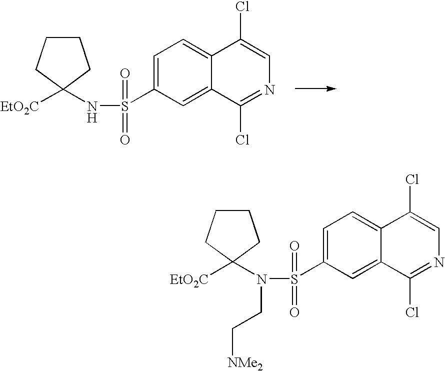

2-(Dimethylamino)ethyl chloride (140 mg, 1.3 mmol) was added to a stirred solution of N-[(1,4-dichloro-7-isoquinolinyl)sulphonyl]cycloleucine ethyl ester (200 mg, 0.48 mmol) and anhydrous K₂CO₃ (80 mg, 0.58 mmol) in DMF (4 mL) under N₂ at 23° C. and the mixture was stirred for 21 h. The cooled mixture was diluted with EtOAc, washed with water , dried (Na₂SO₄), and the solvents were evaporated in vacuo. The residue was dissolved in Et₂O and a solution of HCl in Et₂O (1 M) was added which gave a precipitate. This off-white solid was collected by filtration and dried to give to give N-[(1,4dichloro-7-isoquinolinyl)sulphonyl]-N-[2-(dimethylamino)ethyl]cycloleucine ethyl ester (170 mg, 0.32 mmol).

mp 238-240° C.

¹H (DMSO-d₆, 300 MHz) δ 1.15 (3H, t), 1.55-1.7 (4H, m), 2.0-2.1 (2H, m), 2.2-2.35 (2H, m), 2.8 (6H, s), 3.35-3.45 (2H, m), 3.75-3.85 (2H, m), 4.0 (2H, q), 8.45 (1H, d), 8.5 (1H, d), 8.7 (1H, s) ppm.

LRMS 488, 490 (MH⁺).

Anal. Found: C, 47.53; H, 5.37; N, 7.96. Calc for C₂₁H₂₇Cl₂N₃O₄S.0.25H₂O: C, 47.65; H, 5.43; N, 7.94.

### Preparation 56

### Methyl 1-{[(1,4-dichloro-7-isoquinolinyl)sulphonyl]amino}cyclohexanecarboxylate

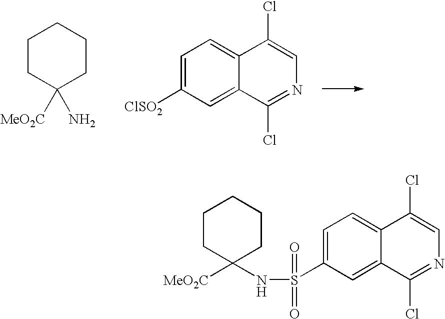

Methyl 1-aminocyclohexanecarboxylate has been prepared previously, see: Didier, E.; Horwell, D. C.; Pritchard, M. C. Tetrahedron, 1992, 48, 8471-8490.

A mixture of methyl 1-aminocyclohexanecarboxylate (325 mg, 1.68 mmol), NEt₃ (0.49 mL, 3.5 mmol) and 1,4-dichloro-7-isoquinolinesulphonyl chloride (415 mg, 1.40 mmol) in CH₂Cl₂ (30 mL) was stirred at 23° C. for 16 h. The mixture was diluted with CH₂Cl₂, washed with dilute HCl (2 M), saturated aqueous NaHCO₃, brine, dried (Na₂SO₄) and evaporated in vacuo. The residue was purified by column chromatography upon silica gel using hexane-EtOAc (80:20 to 70:30) as eluant, followed by trituration with i-Pr₂O, to give to give methyl 1-{[(1,4-dichloro-7-isoquinolinyl)sulphonyl]amino}-cyclohexanecarboxylate (132 mg, 0.32 mmol) as a white solid.

mp 185-186° C.

¹H (CDCl₃, 300 MHz) δ 1.2-1.5 (6H, m), 1.8-2.0 (4H, m), 3.6 (3H, s), 4.95 (1H, s), 8.25 (1H, d), 8.4 (1H, d), 8.5(1H, s), 8.9 (1H, s) ppm.

LRMS 418 (MH⁺).

Anal. Found: C, 48.94; H, 4.43; N, 6.42. Calc for C₁₇H₁₈Cl₂N₂O₄S: C, 48.93; H, 4.35; N, 6.71.

### Preparation 57

### Methyl 4-aminotetrahydro-2H-pyran-4-carboxylate

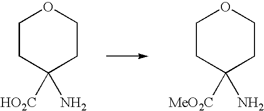

4-Aminotetrahydro-2H-pyran-4-carboxylic acid has been prepared previously, see: Palacin, S.; Chin, D. N.; Simanek, E. E.; MacDonald, J. C.; Whitesides, G. M.; McBride, M. T.; Palmore, G. J. Am. Chem. Soc., 1997, 119, 11807-11816.

A solution 4-aminotetrahydro-2H-pyran-4-carboxylic acid (0.50 g, 3.4 mmol) in MeOH (10 mL) was saturated with HCl gas at 0-5° C., and the mixture was then heated at reflux for 3.5 h. The solvents were evaporated in vacuo, the residue was dissolved in saturated aqueous NaHCO₃ and the aqueous solution was extracted with CH₂Cl₂ (2×50 mL). The combined extracts were dried (MgSO₄) and evaporated in vacuo to give methyl 4-aminotetrahydro-2H-pyran-4-carboxylate (410 mg, 2.58 mmol).

¹H (CDCl₃, 300 MHz) δ 1.4-1.6 (4H, m), 2.05-2.2 (2H, m), 3.6-3.7 (2H, m), 3.75 (3H, s), 3.8-3.9 (2H, m) ppm

LRMS 160 (MH⁺).

### Preparation 58

### Methyl 4-{[(1,4-dichloro-7-isoquinolinyl)sulphonyl]amino}tetrahydro-2H-pyran-4-carboxylate

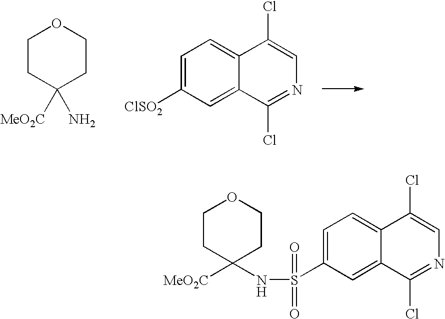

A mixture of methyl 4-aminotetrahydro-2H-pyran-4-carboxylate (400 mg, 2.51 mmol), NEt₃ (0.44 mL, 3.14 mmol) and 1,4-dichloro-7-isoquinolinesulphonyl chloride (621 mg, 2.09 mmol) in CH₂Cl₂ (30 mL) was stirred at 23° C. for 20 h. The mixture was diluted with CH₂Cl₂, washed with dilute HCl (2 M), saturated aqueous NaHCO₃, brine, dried (Na₂SO₄) and evaporated in vacuo. The residue was purified by column chromatography upon silica gel using hexane-EtOAc (80:20) and then CH₂Cl₂-MeOH-0.880NH₃ (95:5:0.5) as eluant, followed by trituration with i-Pr₂O, to give to give methyl 4-{[(1,4-dichloro-7-isoquinolinyl)sulphonyl]amino}tetrahydro-2H-pyran-4-carboxylate (197 mg, 0.47 mmol) as a white solid.

mp 168-170° C.

¹H (CDCl₃, 400 MHz) δ 1.8-1.95 (2H, m), 2.1-2.2 (2H, m), 3.5 (3H, s), 3.5-3.7 (4H, m), 5.4 (1H, s), 8.25 (1H, d), 8.4 (1H, d), 8.5 (1H, s), 8.9 (1H, s) ppm.

LRMS 419 (MH⁺).

Anal. Found: C, 45.97; H, 3.85; N, 6.36. Calc for C₁₆H₁₆Cl₂N₂O₅S: C, 45.83; H, 3.85; N, 6.68.

### Preparation 59

### t-Butyl (±)-cis-2-{[(1,4-dichloro-7-isoquinolinyl)sulphonyl]amino}cyclohexanecarboxylate

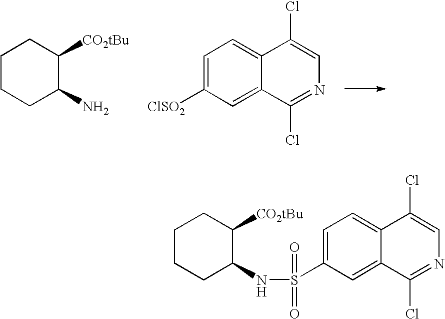

t-Butyl (±)-cis-2-aminocyclohexanecarboxylate has been prepared previously, see: Xie, J.; Soleilhac, J. M.; Renwart, N.; Peyroux, J.; Roques, B. P.; Fournie-Zaluski, M. C. Int. J. Pept. Protein Res 1989, 34, 246-255.

A mixture of t-butyl (±)-cis-2-aminocyclohexanecarboxylate hydrochloride (282 mg, 1.20 mmol), NEt₃ (0.33 mL, 2.37 mmol) and 1,4-dichloro-7-isoquinolinesulphonyl chloride (282 mg, 0.95 mmol) in CH₂Cl₂ (10 mL) was stirred at 23° C. for 1 h. The solvents were evaporated in vacuo and the residue suspended in EtOAc (100 mL). This solution was washed with dilute HCl (10 mL, 1 M), water, dried (MgSO₄) and evaporated in vacuo to give t-butyl (±)-cis-2-{[(1,4-dichloro-7-isoquinolinyl)sulphonyl]amino}cyclohexanecarboxylate (395 mg, 0.86 mmol) as a white solid.

¹H (CDCl₃, 300 MHz) δ 1.1-1.8 (16H, m), 1.95-2.1 (1H, m), 2.5-2.6 (1H, m), 3.4-3.55 (1H, m), 6.1 (1H, d), 8.25 (1H, d), 8.35 (1H, d), 8.45 (1H, s), 8.9 (1H, s).

LRMS 459, 461 (MH⁺). Anal. Found: C, 51.99; H, 5.28; N, 6.01. Calc for C₂₀H₂₄Cl₂N₂O₄S: C, 52.29; H, 5.27; N, 6.10.

### Preparation 60

### Ethyl (±)-cis-2-{[(1,4-dichloro-7-isoquinolinyl)sulphonyl]amino}cyclohexanecarboxylate

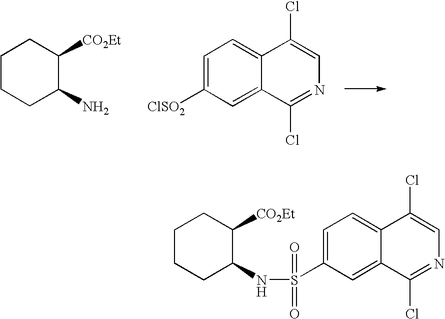

A mixture of ethyl (±)-cis-2-aminocyclohexanecarboxylate hydrochloride (251 mg, 1.20 mmol), NEt₃ (0.33 mL, 2.4 mmol) and 1,4-dichloro-7-isoquinolinesulphonyl chloride (296 mg, 1.00 mmol) in CH₂Cl₂ (10 mL) were stirred at 23° C. for 1 h. The mixture was diluted with CH₂Cl₂ (100 mL), washed with dilute HCl (30 mL, 1 M), water, dried (MgSO₄) and evaporated in vacuo to give ethyl (±)-cis-2-{[(1,4-dichloro-7-isoquinolinyl)sulphonyl]amino}cyclohexanecarboxylate (385 mg, 0.89 mmol) as a white solid.

¹H (CDCl₃, 400 MHz) δ 1.2 (3H, t), 1.2-1.4 (3H, m), 1.4-1.7 (3H, m), 1.75-1.85 (1H, m), 2.0-2.1 (1H, m), 2.65 (1H, q), 3.5-3.6 (1H, m), 3.95-4.0 (1H, m), 4.05-4.15 (1H, m), 5.9 (1H, d), 8.2 (1H, d), 8.35 (1H, d), 8.5 (1H, s), 8.9 (1H, s).

LRMS 431, 433 (MH⁺).

Anal. Found: C, 50.45; H, 4.79; N, 6.31. Calc for C18H₂₀Cl₂N₂O₄S: C, 50.12; H, 4.67; N, 6.49.

### Preparation 61

### t-Butyl cis-4-{[(1,4-dichloro-7-isoquinolinyl)sulphonyl]amino}cyclohexanecarboxylate

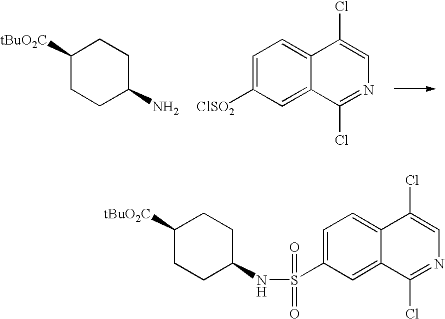

t-Butyl cis-4-aminocyclohexanecarboxylate has been prepared previously, see: Barnish, I. T.; James, K.; Terrett, N. K.; Danilewicz, J. C.; Samuels, G. M. R.; Wythes, M. J. Eur. Patent, 1988, EP 274234.

A mixture of t-butyl cis-4-aminocyclohexanecarboxylate (282 mg, 1.20 mmol), NEt₃ (0.33 mL, 2.37 mmol) and 1,4-dichloro-7-isoquinolinesulphonyl chloride (296 mg, 1.00 mmol) in CH₂Cl₂ (10 mL) was stirred at 0° C. for 1 h. The mixture was diluted with CH₂Cl₂ (150 mL), was washed with dilute HCl (30 mL, 1 M), water, dried (MgSO₄) and evaporated in vacuo. The residue was purified by column chromatography upon silica gel using pentane-EtOAc (100:0 to 75:25) to give t-butyl cis4-{[(1,4-dichloro-7-isoquinolinyl)sulphonyl]aminocyclohexanecarboxylate (360 mg, 0.78 mmol) as a white solid.

¹H (CDCl₃, 400 MHz) δ 1.4 (9H, s), 1.5-1.65 (6H, m), 1.75-1.85 (2H, m), 2.3 (1H, m), 3.45 (1H, m), 4.75 (1H, d), 8.25 (1H, d), 8.4 (1H, d), 8.5 (1H, s), 8.9 (1H, s) ppm.

LRMS 459, 461 (MH⁺), 476 (MNH₄⁺).

Anal. Found: C, 52.34; H, 5.28; N, 5.98. Calc for C₂₀H₂₄Cl₂N₂O₄S: C, 52.29; H, 5.27; N, 6.10.

### Preparation 62

### Ethyl trans-4-{[(1,4-dichloro-7-isoquinolinyl)sulphonyl]amino}cyclohexanecarboxylate

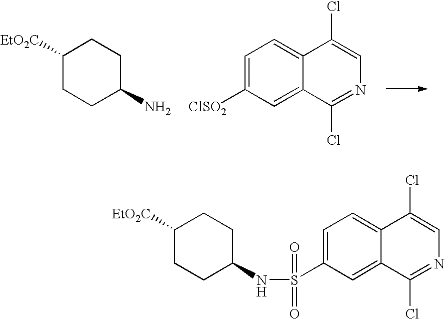

Ethyl trans-4-aminocyclohexanecarboxylate has been prepared previously, see: Skaric, V.; Kovacevic, M.; Skaric, D. J. Chem. Soc., Perkin Trans. 11976, 1199-1201.

A mixture of ethyl trans-4-aminocyclohexanecarboxylate (168 mg, 0.81 mmol), NEt₃ (0.22 mL, 1.6 mmol) and 1,4-dichloro-7-isoquinolinesulphonyl chloride (200 mg, 0.67 mmol) in CH₂Cl₂ (8 mL) was stirred at 0° C. for 1 h. The mixture was diluted with CH₂Cl₂ (100 mL), was washed with dilute HCl (50 mL, 1 M), water, dried (MgSO₄) and evaporated in vacuo to give ethyl trans-4-{[(1,4-dichloro-7-isoquinolinyl)sulphonyl]amino}cyclohexanecarboxylate (232 mg, 0.54 mmol) as a white solid.

¹H (CDCl₃, 400 MHz) δ 1.15-1.3 (5H, m), 1.4-1.55 (2H, m), 1.9-2.0 (4H, m), 2.1-2.2 (1H, m), 3.2-3.3 (1H, m), 4.1 (2H, t), 4.55 (1H, d), 8.25 (1H, d), 8.35 (1H, d), 8.5 (1H, s), 8.9 (1H, s)

LRMS 431 (MH⁺).

### Preparation 63

### 1,4-Dichloro-7-isoquinolinecarbonyl chloride

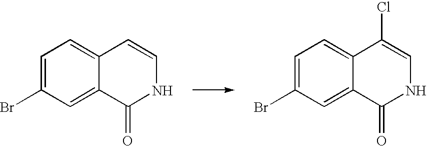

A solution of N-chlorosuccinimide (4.13 g, 31 mmol) in MeCN (50 mL) was added dropwise to a stirred solution of 7-bromo-1-(2H)-isoquinolone (6.6 g, 29.5 mmol) in MeCN (150 mL) which was heating under reflux. The mixture was heated under reflux for an additional 3 h and then cooled to room temperature. The resulting precipitate was collected by filtration, with MeCN rinsing, and then dried in vacuo to give 7-bromo-4-chloro-1(2H)-isoquinolone (6.72 g, 26.0 mmol) as a white solid.

mp 241-243° C.

¹H (DMSO-d₆, 300 MHz) δ 7.5 (1H, s), 7.73 (1H, d), 7.8 (1H, dd), 8.3 (1H, s) ppm.

LRMS 259 (MH⁺), 517 (M₂H⁺).

Anal. Found: C, 41.69; H, 1.90; N, 5.37. Calc for C₉H₅BrClNO: C, 41.80; H, 1.95; N, 5.42.

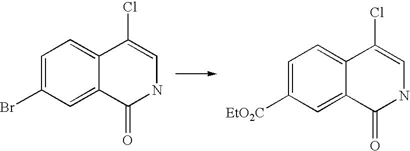

A mixture of 7-bromo-4-chloro-1(2M)-isoquinolone (1.0 g, 3.87 mmol) and bis(triphenylphosphine) palladium (II) chloride (100 mg, 0.14 mmol) in EtOH (15 mL) and NEt₃ (2 mL) was heated to 100° C. in a pressure vessel under an atmosphere of CO (100 psi) for 48 h. After cooling and venting the vessel, the catalyst was removed by filtration, and the filtrate was evaporated in vacuo. The residue was purified by column chromatography upon silica gel using hexane-EtOAc (50:50) as eluant, and then by crystallisation from i-Pr₂O. This material was combined with CH₂Cl₂ washings of the catalyst residues to give ethyl 4-chloro-1-oxo-1,2-dihydro-7-isoquinolinecarboxylate (743 mg, 2.95 mmol) as a white solid.

mp 184-186° C.

¹H (CDCl₃, 300 MHz) δ 1.45 (2H, t), 4.45 (2H, q), 7.4 (1H, s), 7.95 (1H, d), 8.4 (1H, d), 9.05 (1H, s) ppm.

LRMS 252 (MH⁺), 269 (MNNH₄⁺), 503 (M₂H⁺).

Anal. Found: C, 57.02; H, 3.99; N, 5.53. Calc for C₁₂H₁₀CtNO₃: C, 57.27; H, 4.01; N, 5.57.

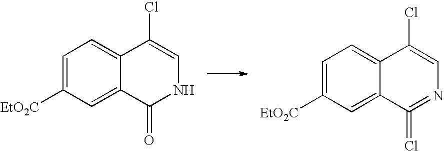

Ethyl 4-chloro-1-oxo-1,2-dihydro-7-isoquinolinecarboxylate (500 mg, 1.99 mmol) was warmed in POCl₃ (3 mL) until a clear solution formed, and was then allowed to stand at 23° C. for 18 h. The reaction mixture was poured into warm water, extracted with EtOAc (3×20 mL), and the combined organic extracts washed with water and saturated brine, dried (MgSO₄), and evaporated in vacuo. The residue was purified by column chromatography upon silica gel using hexane-EtOAc (90:10) as eluant followed by crystallisation from i-Pr₂O to give ethyl 1,4-dichloro-7-isoquinolinecarboxylate (377 mg, 1.40 mmol) as a pale pink solid.

mp 92-94° C.

¹H (CDCl₃, 300 MHz) δ 1.45 (2H, t), 4.45 (2H, q), 8.25 (1H, d), 8.4-8.45 (2H, m), 9.05 (1H, s) ppm.

LRMS 270 (MH⁺).

Anal. Found: C, 53.27; H, 3.48; N, 5.14. Calc for C₁₂H₉Cl₂NO₂: C, 53.36; H, 3.36; N, 5.19.

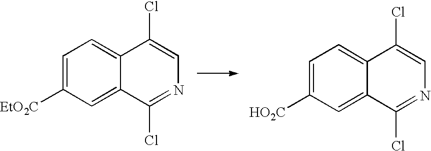

Ethyl 1,4-dichloro-7-isoquinolinecarboxylate (500 mg, 1.85 mmol) in THF (2 mL) was treated with an aqueous solution of NaOH (3.7 mL, 1 M) and EtOH (few drops) added to give a single phase mixture. After stirring at room temperature overnight, HCl (3.7 mL, 1 M) was added to give a thick slurry which was filtered off, washed with water, and crystallised from i-PrOH. The fluffy white crystalline solid was triturated with hexane and dried to afford 1,4-dichloro-7-isoquinolinecarboxylic acid (240 mg, 0.99 mmol).

mp 226-228° C.

¹H (DMSO-d₆, 300 MHz) δ 8.3 (1H, d), 8.4 (1H, d), 8.55 (1H, s), 8.8 (1H, s) ppm.

LRMS 242 (MH⁺).

Anal. Found: C, 49.59; H, 2.08; N, 5.74. Calc for C₁₀H₅Cl₂NO₂: C, 49.62; H, 2.08; N, 5.78.

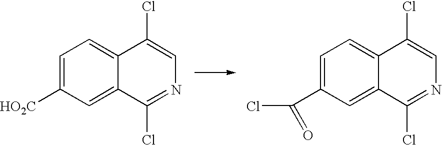

Oxalyl chloride (144 μL, 1.65 mmol) was added to a suspension of 1,4-dichloro-7-isoquinolinecarboxylic acid (200 mg, 0.83 mmol) at room temperature in CH₂Cl₂ (10 mL), followed by DMF (1 drop). After 30 min the resultant clear solution was evaporated in vacuo to afford 1,4-dichloro-7-isoquinolinecarbonyl chloride which was used without further purification.

### Preparation 64

### N-[(1,4-Dichloro-7-isoquinolinyl)carbonyl]glycine t-butyl ester

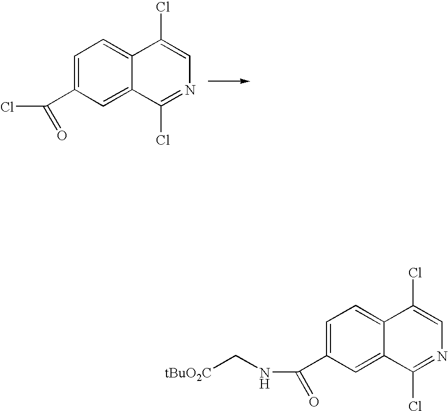

A solution of 1,4-dichloro-7-isoquinolinecarbonyl chloride (213 mg, 0.8 mmol) in CH₂Cl₂ (10 mL) was added to a stirred suspension of glycine t-butyl ester hydrochloride (166 mg, 0.99 mmol) and NEt₃ (253 μL, 1.82 mmol) in CH₂Cl₂ (5 mL). The reaction mixture was stirred at room temperature overnight, quenched with a drop of water and then evapourated in vacuo. The residue was purified by column chromatography upon silica gel using hexane-EtOAc (70:30) as eluant to give N-[(1,4-dichloro-7-isoquinolinyl)carbonyl]glycine t-butyl ester (140 mg, 0.39 mmol). An analytical sample was prepared by crystallisation from i-Pr₂O—CH₂Cl₂.

mp 162-164° C.

¹H (CDCl₃, 300 MHz) δ 1.5 (9H, s), 4.15-4.2 (2H, m), 6.9 (1H, s), 8.25-8.3 (2H, m), 8.4 (1H, s), 8.75 (1H, s) ppm.

LRMS 355 (MH⁺).

Anal. Found: C, 53.98; H, 4.36; N, 7.83. Calc for C₁₆H₁₆Cl₂N₂O₃: C, 54.10; H, 4.54; N, 7.89.

### Preparation 65

### N-[(1,4-Dichloro-7-isoquinolinyl)carbonyl]-β-alanine t-butyl ester

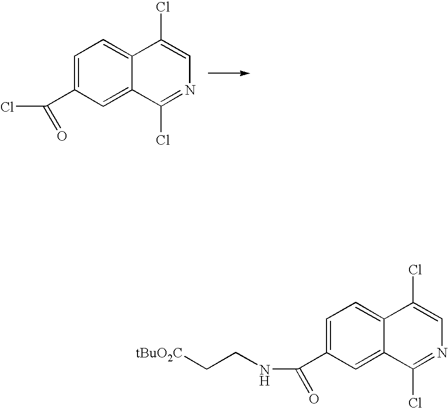

A solution of 1,4-dichloro-7-isoquinolinecarbonyl chloride (450 mg, 1.7 mmol) in CH₂Cl₂ (20 mL) was added to a stirred solution of β-alanine t-butyl ester hydrochloride (376 mg, 2.07 mmol) and NEt₃ (530 μL, 3.81 mmol) in CH₂Cl₂ (10 mL) and the mixture was stirred at room temperature for 3 h. The mixture was washed with HCl (2×30 mL, 1 M), aqueous NaHCO₃ (10%, 30 mL), dried (Na₂SO₄), and evaporated in vacuo. The residue was crystallised from i-Pr₂O to give N-[(1,4-dichloro-7-in vacuo. The residue was crystallised from i-Pr₂O to give N-[(1,4-dichloro-7-isoquinolinyl)carbonyl]cycloleucine ethyl ester (372 mg, 0.98 mmol) as a white solid.

mp 178-180° C.

¹H (CDCl₃, 300 MHz) δ 1.3 (3H, t), 1.8-2.05 (4H, m), 2.1-2.3 (2H, m), 2.3-2.45 (2H, m), 4.25 (2H, q), 6.95 (1H, br s), 8.2-8.25 (2H, m), 8.4 (1H, s), 8.7(1H, s) ppm.

LRMS 382 (MH⁺), 398 (MNH₄⁺), 763 (M₂H⁺).

Anal. Found: C, 56.71; H, 4.77; N, 7.27. Calc for C₁₈H₁₈Cl₂N₂O₃: C, 56.70; H, 4.76; N, 7.35.

### Preparation 67

### N-[(1,4-Dichloro-7-isoquinolinyl)carbonyl]-DL-phenylglycine t-butyl ester

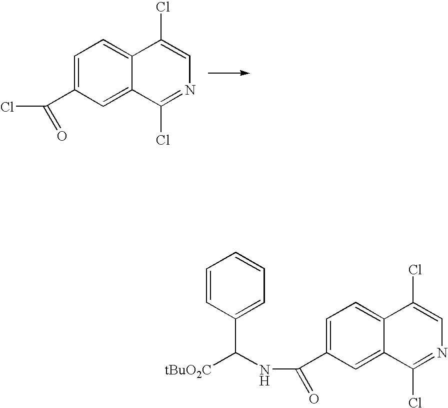

A solution of 1,4-dichloro-7-isoquinolinecarbonyl chloride (450 mg, 1.73 mmol) in CH₂Cl₂ (20 mL) was added to a stirred solution of DL-phenylglycine t-butyl ester hydrochloride (505 mg, 2.07 mmol) and NEt₃ (530 μL, 3.81 mmol) in CH₂Cl₂ (30 mL) and the mixture was stirred at room temperature for 3 h. The mixture was washed with dilute HCl (2×30 mL, 1 M), aqueous NaHCO₃ (10%), dried (Na₂SO₄), and evaporated in vacuo to give N-[(1,4-dichloro-7-isoquinolinyl)carbonyl]-DL-phenylglycine t-butyl ester (600 mg, 1.39 mmol) as a waxy solid. An analytical sample was prepared by the slow evaporation of a solution in CH₂Cl₂ to give a fluffy white solid.

mp 146-149° C.

¹H (CDCl₃, 300 MHz) δ 1.5 (9H, s), 5.7 (1H, d), 7.3-7.5 (6H, m), 8.2-8.3 (2H, m), 8.4 (1H, s), 8.8 (1H, s) ppm.

LRMS 431 (MH⁺), 861 (M₂H⁺).

Anal. Found: C, 60.57; H, 4.76; N, 6.42. Calc for C₂₂H₂₀Cl₂N₂O₃.0.25H₂O: C, 60.63; H, 4.74; N, 6.43.

### Preparation 68

N-[(1,4-Dichloro-7-isoquinolinyl)carbonyl]-L-phenylglycine t-butyl ester

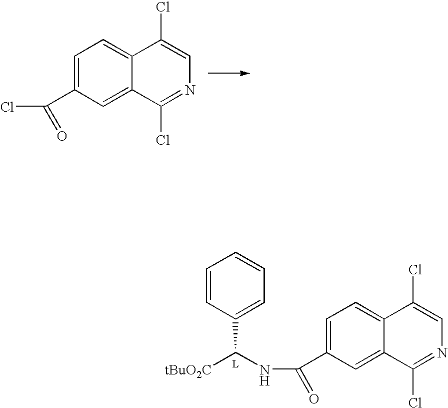

A solution of 1,4-dichloro-7-isoquinolinecarbonyl chloride (148 mg, 0.57 mmol) in CH₂Cl₂ (6 mL) was added to a stirred solution of S-(+)-phenylglycine t-butyl ester hydrochloride (138 mg, 0.57 mmol) and NEt₃ (200 μL, 1.44 mmol) in CH₂Cl₂ (5 mL), and the mixture was stirred at room temperature overnight. The mixture was diluted with CH₂Cl₂ (25 mL), washed with dilute HCl (0.5 M), aqueous NaHCO₃ (10%), brine, dried (Na₂SO₄), and evaporated in vacuo to give N-[(1,4-dichloro-7-isoquinolinyl)carbonyl]-L-phenylglycine t-butyl ester (218 mg, 0.51 mmol) as a gum. An analytical sample was prepared by trituration with hexane yielding a solid.

mp 173-175° C.

¹H (CDCl₃, 300 MHz) δ 1.45 (9H, s), 5.7 (1H, d), 7.3-7.5 (6H, m), 8.25 (2H, s), 8.4 (1H, s), 8.8 (1H, s) ppm.

LRMS 431 (MH⁺), 448 (MNH₄⁺), 861 (M₂H⁺), 883 (M₂Na⁺).

Anal. Found: C, 58.83; H, 4.88; N, 5.90. Calc for C₂₂H₂₀Cl₂N₂O₃.H₂O: C, 58.80; H, 4.93; N, 6.23.

### Preparation 69

### N-[(1,4-Dichloro-7-isoquinolinyl)carbonyl]-D-phenylglycine t-butyl ester

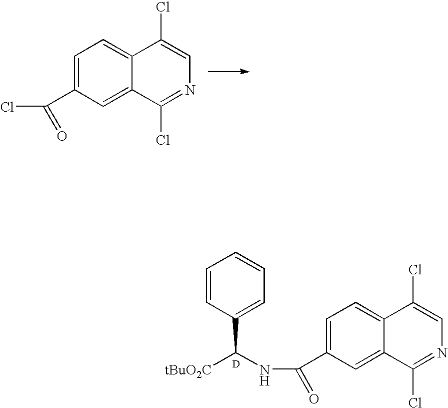

A solution of 1,4-dichloro-7-isoquinolinecarbonyl chloride (148 mg, 0.57 mmol) in CH₂Cl₂ (6 mL) was added to a stirred solution of R-(+)-phenylglycine t-butyl ester hydrochloride (138 mg, 0.57 mmol) and NEt₃ (200 μL, 1.44 mmol) in CH₂Cl₂ (5 mL), and the mixture was stirred at room temperature overnight. The mixture was diluted with CH₂Cl₂ (25 mL), washed with dilute HCl (0.5 M), aqueous NaHCO₃ (10%), brine, dried (Na₂SO₄), and evaporated in vacuo. Trituration of the residue with hexane gave N-[(1,4-dichloro-7-isoquinolinyl)carbonyl]-D-phenylglycine t-butyl ester (203 mg, 0.47 mmol) as a white solid.

¹H (CDCl₃, 300 MHz) δ 1.4 (9H, s), 5.7 (1H, d), 7.3-7.5 (6H, m), 8.25 (2H, s), 8.4 (1H, s), 8.8 (1H, s) ppm.

LRMS 431 (MH⁺), 448 (MNH₄⁺), 861 (M₂H⁺), 883 (M₂Na⁺).

Anal. Found: C, 61.17; H, 4.70; N, 6.37. Calc for C₂₂H₂₀Cl₂N₂O₃: C, 61.26; H, 4.67; N, 6.50.

### Preparation 70

### N-[(1,4-Dichloro-7-isoquinolinyl)carbonyl]-DL-valine t-butyl ester

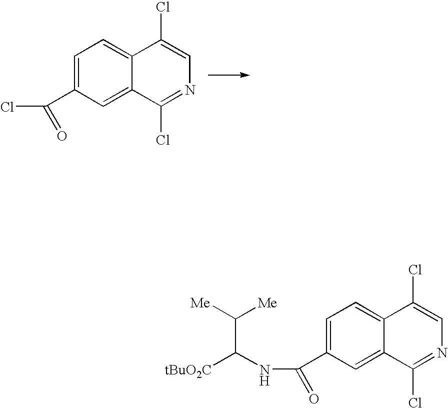

A solution of 1,4-dichloro-7-isoquinolinecarbonyl chloride (450 mg, 1.73 mmol) in CH₂Cl₂ (20 mL) was added to a stirred solution of DL-valine t-butyl ester hydrochloride (435 mg, 2.07 mmol) and NEt₃ (530 μL, 3.81 mmol) in CH₂Cl₂ (10 mL) and the mixture was stirred at room temperature for 3 h. The mixture was washed with dilute HCl (1 M), aqueous NaHCO₃ (10%), dried (Na₂SO₄), and evaporated in vacuo. The residue was crystallised with i-Pr₂O to give N-[(1,4-dichloro-7-isoquinolinyl)carbonyl]-DL-valine t-butyl ester (390 mg, 0.98 mmol) as a white solid.

¹H (CDCl₃, 400 MHz) δ 1.0-1.05 (6H, m), 1.5 (9H, s), 2.3-2.4 (1H, m), 4.7-4.8 (1H, m), 6.85 (1H, d), 8.25-8.3 (2H, m), 8.4 (1H, s), 8.75 (1H, s) ppm.

LRMS 397 (MH⁺), 793 (M₂H⁺).

Anal. Found: C, 57.20; H, 5.53; N, 6.99. Calc for C₁₉H₂₂C₁₂N₂O₃: C, 57.44; H, 5.58; N, 7.05.

### Preparation 71

### N-[(1,4-Dichloro-7-isoquinolinyl)carbonyl]-DL-proline t-butyl ester

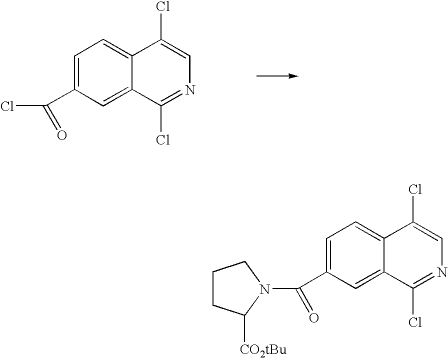

DL-Proline t-butyl ester hydrochloride (320 mg, 1.54 mmol) and then NEt₃ (513 μL, 3.69 mmol) were added to a stirred solution of 1,4-dichloro-7-isoquinolinecarbonyl chloride (270 mg, 1.04 mmol) in CH₂Cl₂ (32 mL) and the cloudy solution was then stirred at room temperature for 4 h. The mixture was diluted with CH₂Cl₂ (20 mL), washed with dilute HCl (1 M), saturated brine, dried (Na₂SO₄), and evaporated in vacuo. The residue was crystallised with i-Pr₂O to give N-[(1,4-dichloro-7-isoquinolinyl)carbonyl]-DL-proline r-butyl ester (395 mg, 1.00 mmol) as a white solid.

mp 144-146° C.

¹H (CDCl₃, 300 MHz) shows a 3:1 mixture of rotamers δ 1.15 (1/4 of 9H, s), 1.55 (3/4 of 9H, s), 1.8-2.15 (3H, m), 2.2-2.4 (1H, m), 3.45-3.9 (2H, m), 4.2-4.3 (1/4 of 1H, m), 4.6-4.7 (3/4 of 1H, m), 7.9 (1/4 of 1H, d), 8.05 (3/4 of 1H, d), 8.2-8.3 (1H, m), 8.4 (1H, s), 8.55 (1H, s) ppm.

LRMS 395 (MH⁺), 789 (M₂H⁺).

Anal. Found: C, 57.79; H, 5.11; N, 6.97. Calc for C₁₉H₂₀Cl₂N₂O₃: C, 57.73; H, 5.10; N, 7.09.

### Preparation 72

### N-[(1,4-Dichloro-7-isoquinolinyl)carbonyl]-DL-phenylalanine t-butyl ester

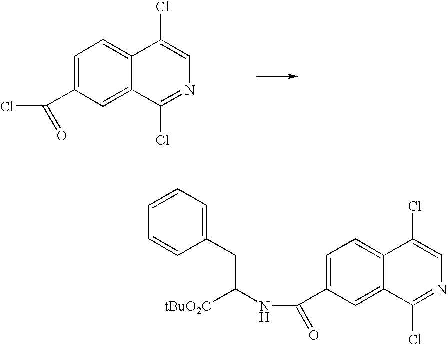

A mixture of NEt₃ (330 μL, 2.37 mmol), DL-phenylalanine t-butyl ester hydrochloride (293 mg, 1.14 mmol) and 1,4-dichloro-7-isoquinolinecarbonyl chloride (247 mg, 0.95 mmol) in CH₂Cl₂ (20 mL) was stirred at room temperature for 18 h. The solvents were evaporated in vacuo and the residue partioned between dilute HCl (1M) and EtOAc. The organic phase was washed with brine, dried (Na₂SO₄) and evaporated in vacuo. The residue was crystallised with i-Pr₂O to give N-[(1,4-dichloro-7-isoquinolinyl)carbonyl]-DL-phenylalanine t-butyl ester (384 mg, 0.86 mmol) as a white solid.

mp 156-157° C.

¹H (CDCl₃, 300 MHz) δ 1.5 (9H, s), 3.2:3.3 (2H, m), 5.0 (1H, dt), 6.8 (1H, d), 7.2-7.49 (5H, m), 8.2 (1H, d), 8.25 (1H, d), 8.4 (1H, s), 8.6 (1H, s) ppm.

LRMS 445 (MH₄⁺).

Anal. Found: C, 62.02; H, 4.98; N, 6.28. Calc for C₂₃H₂₂Cl₂N₂O₃: C, 62.03; H, 4.98; N, 6.29.

### Preparation 73

### N-[(1,4-Dichloro-7-isoquinolinyl)carbonyl]-DL-leucine t-butyl ester

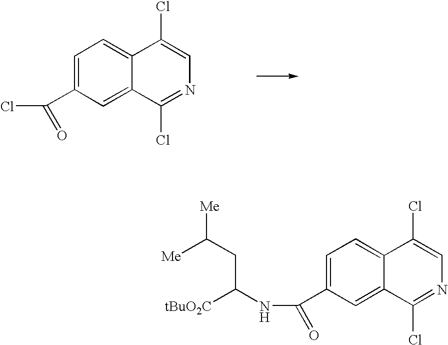

A solution of 1,4-dichloro-7-isoquinolinecarbonyl chloride (247 mg, 0.95 mmol) in CH₂Cl₂ (10 mL) was added to a solution of DL-leucine t-butyl ester hydrochloride (255 mg, 1.14 mmol) and NEt₃ (330 μL, 2.37 mmol) in CH₂Cl₂ (10 mL) and the mixture was stirred at room temperature overnight. The solvents were evaporated in vacuo and the residue was partioned between dilute HCl (1 M) and EtOAc. The organic phase was washed with brine, dried (Na₂SO₄) and evaporated in vacuo. The residue was crystallised with i-Pr₂O to give N-[(1,4-dichloro-7-isoquinolinyl)carbonyl]-DL-leucine t-butyl ester (285 mg, 0.69 mmol).

mp 183-184° C.

¹H (CDCl₃, 300 MHz) δ 1.0-1.1 (6H, m), 1.5 (9H, s), 1.65-1.85 (3H, m), 4.75-4.85 (1H, m), 6.8 (1H, d), 8.2 (2H, s), 8.4 (1H, s), 8.7 (1H, s) ppm.

LRMS 411 (MH⁺).

Anal. Found: C, 58.39; H, 5.84; N, 6.76. Calc for C₂₀H₂₄Cl₂N₂O₃: C, 58.40; H, 5.88; N, 6.81.

### Preparation 74

### t-Butyl DL-3-{[(1,4-dichloro-7-isoquinolinyl)carbonyl]amino}-3-phenylpropanoate

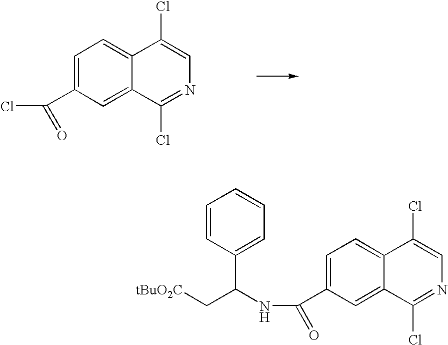

A solution of 1,4-dichloro-7-isoquinolinecarbonyl chloride (247 mg, 0.95 mmol) in CH₂Cl₂ (10 mL) was added to a solution of DL-3-amino-3-phenylpropionic acid t-butyl ester (252 mg, 1.14 mmol) and NEt₃ (260 μL, 1.87 mmol) in CH₂Cl₂ (10 mL) and the mixture was stirred at room temperature overnight. The solvents were evaporated in vacuo and the residue was partioned between dilute HCl (1 M) and EtOAc. The organic phase was washed with brine, dried (Na₂SO₄) and evaporated in vacuo to give t-butyl DL-3-{[(1,4-dichloro-7-isoquinolinyl)carbonyl]amino}-3-phenylpropanoate (323 mg, 0.73 mmol). An analytical sample was prepared by crystallisation with i-Pr₂O-hexane to yield a white powder.

mp 153-155° C.

¹H (CDCl₃, 300 MHz) δ 1.4 (9H, m), 2.9-3.05 (2H, m), 5.6 (1H, dt), 7.2-7.4 (5H, m), 7.9 (1H, d), 8.2 (2H, s), 8.4 (1H, s), 8.7 (1H, s) ppm.

LRMS 445 (MH⁺).

Anal. Found: C, 61.99; H, 5.07; N, 6.15. Calc for C₂₃H₂₂Cl₂N₂O₃: C, 62.03; H, 4.98; N, 6.29.

### Preparation 75

### N-[(1,4-Dichloro-7-isoquinolinyl)carbonyl]-DL-aspartic acid α,β-di-t-butyl ester

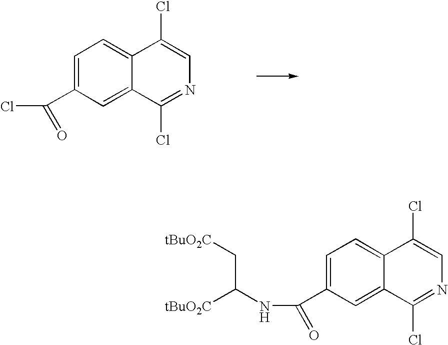

A solution of 1,4-dichloro-7-isoquinolinecarbonyl chloride (247 mg, 0.95 mmol) in CH₂Cl₂ (10 mL) was added to a solution of aspartic acid α,β-di-t-butyl ester hydrochloride (321 mg, 1.14 mmol) and NEt₃ (330 μL, 2.37 mmol) in CH₂Cl₂ (10 mL) and the mixture was stirred at room temperature overnight. The mixture was diluted with CH₂Cl₂ (30 mL), washed with dilute HCl (3×30 mL, 1 M), saturated aqueous Na₂CO₃, brine, dried (MgSO₄) and evaporated in vacuo. The residue was crystallised from hexane to give, in two crops, N-[(1,4-dichloro-7-isoquinolinyl)carbonyl)-DL-aspartic acid α,β-di-t-butyl ester (298+88 mg, 0.63+0.19 mmol) as a fluffy white solid.

mp 112-114° C.

¹H (CDCl₃, 300 MHz) δ 1.45 (9H, m), 1.55 (9H, m), 2.9 (1H, dd), 3.05 (1H, dd), 4.9-5.0 (1H, m), 7.45 (1H, d), 8.25-8.35 (2H, m), 8.45 (1H, s), 8.75 (1H, s) ppm.

LRMS 469 (MH⁺), 491 (MNa⁺), 959 (M₂Na⁺).

Anal. Found: C, 56.20; H, 5.57; N, 5.88. Calc for C₂₂H₂₆Cl₂N₂O₅: C, 56.29; H, 5.58; N, 5.97.

### Preparation 76

### O-t-Butyl-N-[(1,4-dichloro-7-isoquinolinyl)carbonyl]-DL-serine t-butyl ester

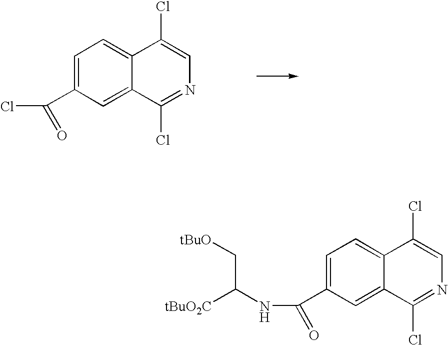

A solution of 1,4-dichloro-7-isoquinolinecarbonyl chloride (247 mg, 0.95 mmol) in CH₂Cl₂ (10 mL) was added to a solution of O-t-butyl-DL-serine t-butyl ester hydrochloride (288 mg, 1.14 mmol) and NEt₃ (330 L, 2.37 mmol) in CH₂Cl₂ (10 mL) and the mixture was stirred at room temperature for 3 h. The mixture was diluted with CH₂Cl₂ (30 mL), washed with HCl (1 M), saturated aqueous Na₂CO₃, saturated brine, dried (Na₂SO₄) and evaporated in vacuo. The residue was crystallised from hexane to give O-t-butyl-N-[(1,4-dichloro-7-isoquinolinyl)carbonyl]-DL-serine t-butyl ester (378 mg, 0.86 mmol) as a white solid.

mp 116-117° C.

¹H (CDCl₃, 300 MHz) δ 1.1 (9H, m), 1.5 (9H, m), 3.7 (1H, dd), 3.9 (1H, dd), 4.8-4.9 (1H, m), 7.15 (1H, d), 8.25-8.35 (2H, m), 8.4 (1H, s), 8.75 (1H, s) ppm.

LRMS 441 (MH⁺), 881 (M₂H⁺), 903 (M₂Na⁺).

Anal. Found: C, 57.15; H, 5.94; N, 6.27. Calc for C₂₁H₂₆Cl₂N₂O₄: C, 57.15; H, 5.94; N, 6.35.

### Preparation 77

### N-[(1,4-Dichloro-7-isoquinolinyl)carbonyl]-DL-(α-cyclopentylglycine t-butyl ester

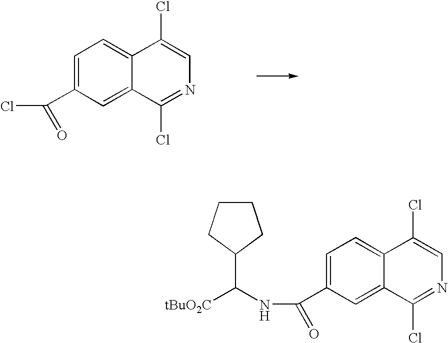

A solution of 1,4-dichloro-7-isoquinolinecarbonyl chloride (148 mg, 0.57 mmol) in CH₂Cl₂ (6 mL) was added to a solution of DL-α-cyclopentylglycine t-butyl ester hydrochloride (134 mg, 0.57 mmol) and NEt₃ (200 μL, 1.44 mmol) in CH₂Cl₂ (5 mL) and the mixture was stirred at room temperature overnight. The reaction mixture was diluted with CH₂Cl₂ (25 mL), washed with dilute HCl (0.5 M), saturated aqueous Na₂CO₃, brine, dried (Na₂SO₄) and evaporated in vacuo. The residue was crystallised from i-Pr₂O-hexane to give N-[(1,4-dichloro-7-isoquinolinyl)carbonyl]-DL-α-cyclopentylglycine t-butyl ester (198 mg, 0.47 mmol) as a white solid.

¹H (CDCl₃, 300 MHz) δ 1.4-1.9 (17H, m), 2.3-2.5 (1H, m), 4.8 (1H, dd), 6.85 (1H, d), 8.2-8.3 (2H, m), 8.4 (1H, s), 8.7 (1H, s) ppm.

LRMS 423 (MH⁺), 440 (MNH₄⁺), 445 (MNa⁺), 845 (M₂H⁺), 867 (M₂Na⁺).

Anal. Found: C, 59.56; H, 5.72; N, 6.57. Calc for C₂₁H₂₄Cl₂N₂O₃: C, 59.58; H, 5.72; N, 6.62.

### Preparation 78

### N-Benzyl-N-[(1,4-dichloro-7-isoquinolinyl)carbonyl]glycine t-butyl ester

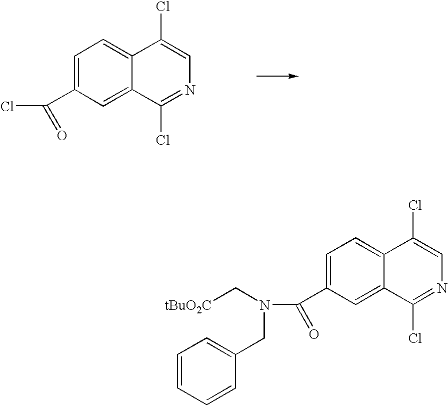

Oxalyl chloride (95 μl, 1.09 mmol) and then DMF (2 drops) were added to a stirred suspension of 1,4-dichloro-7-isoquinolinecarboxylic acid (130 mg, 0.54 mmol) in CH₂Cl₂ (10 mL), and the mixture was stirred for 30 min. to give a clear solution of the corresponding acid chloride. The solvents were evaporated in vacuo and the residue redissolved in CH₂Cl₂ (10 mL). N-Benzylglycine t-butyl ester hydrochloride (152 mg, 0.59 mmol) and NEt₃ (200 μL, 1.44 mmol) were added and the mixture stirred at room temperature overnight. The solvents were evaporated in vacuo, and the residue was partioned between Et₂O and dilute HCl (1 M). The organic phase was washed with dilute HCl (1 M), aqueous Na₂CO₃ (10%, 20 mL), saturated brine, dried (Na₂SO₄), and evaporated in vacuo. The residue was extracted with hot hexane, and the organic solution was decanted from the insoluble material. The organic solution was evaporated in vacuo and the residue purified by column chromatography upon silica gel using hexane-EtOAc (80:20) as eluant to give N-benzyl-N-[(1,4-dichloro-7-isoquinolinyl)carbonyl]glycine t-butyl ester (130 mg, 0.29 mmol) as an oil.

¹H (CDCl₃, 400 MHz) shows a 1:2 mixture of rotamers δ 1.4 (1/3 of 9H, s), 1.5 (2/3 of 9H, s), 3.75 (1/3 of 2H, s), 4.1 (2/3 of 2H, s), 4.6 (2/3 of 2H, s), 4.85 (1/3 of 2H, s), 7.2-7.45 (5H, m), 7.9-8.05 (1H, m), 8.2-8.5 (3H, m) ppm.

LRMS 445 (MH⁺), 467 (MNa⁺), 889 (M₂H⁺), 911 (M₂Na⁺).

### Preparation 79

### 7-(Chloromethyl)-1,4-dichloro-isoquinoline

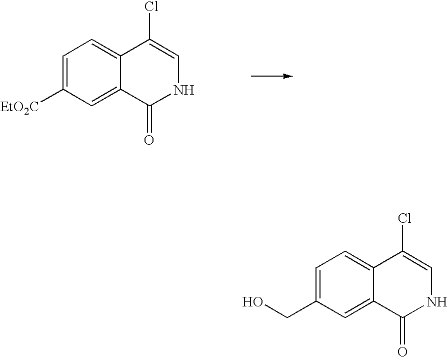

LiBH₄ (530 mg, 24.3 mmol) was added portionwise to a stirred solution of ethyl 4-chloro-1-oxo-1,2-dihydro-7-isoquinolinecarboxylate (3.06 g, 12.2 mmol) in THF (100 mL) and the mixture was stirred at room temperature for 1 h. The heterogeneous mixture was quenched with dilute HCl (2 M), and extracted with CH₂Cl₂ (2×100 mL) and EtOAc (5×100 mL). The remaining solid was taken up in hot EtOH, and allowed to cool to yield a white fluffy solid. This solid was combined with the combined organic extracts, evaporated in vacuo and crystallised with EtOH to give 4-chloro-7-(hydroxymethyl)-1(2H)-isoquinolone (2.19 g, 10.49 mmol) as a white solid.

mp 266-268° C. ¹H (DMSO-d₆, 300 MHz) δ 4.6 (2H, d), 5.4 (1H, t), 7.4 (1H, s), 7.7-7.8 (2H, m), 8.2 (1H, s) ppm.

LRMS 210 (MH⁺), 419 (M₂H⁺).

Anal. Found: C, 57.1 1; H, 3.8 1; N, 6.54. Calc for C₁₀H₈ClNO₂: C, 57.29; H, 3.85; N, 6.68.

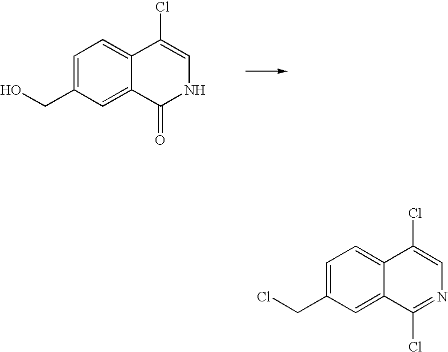

A solution of 4-chloro-7-(hydroxymethyl)-1(2H)-isoquinolone (1.00 g, 4.77 mmol) in POCl₃ was stirred at 50° C. for 19 h. The reaction mixture was cooled in an ice-bath, quenched by the dropwise addition of dilute HCl (1 M) (reaction temperature <30° C.) and then partioned between water and EtOAc. The aqueous phase was reextracted with EtOAc and the combined organic extracts were dried (Na₂SO₄) and evaporated in vacuo. The residue was purified by column chromatography upon silica gel using hexane-EtOAc (80:20) as eluant to give 7-(chloromethyl)-1,4-dichloroisoquinoline (870 mg, 3.53 mmol).

mp 139-141° C.

¹H (CDCl₃, 400 MHz) δ 4.8 (2H, s), 7.9 (1H, d), 8.1 (1H, d), 8.3-8.4 (2H, m) ppm.

LRMS 241 [C₁₁H₉Cl₂ON—H⁺; product of MeO (from MeOH) substitution of Cl]

### Preparation 80

### N-[(1,4-Dichloro-7-isoquinolinyl)methyl]-N-methyl-DL-phenylglycine t-butyl ester

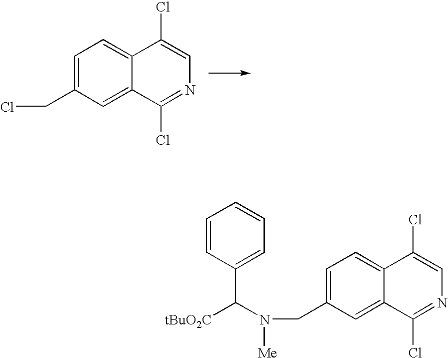

7-(Chloromethyl)-1,4-dichloroisoquinoline (230 mg, 0.93 mmol) was added to a solution of N-methyl-DL-phenylglycine t-butyl ester (248 mg, 0.96 mmol) and NEt₃ (187 μL, 1.34 mmol) in CH₂Cl₂ (5 mL), and the mixture heated at reflux for 15 h. [TLC indicated incomplete reaction]. The solvent was evaporated in vacuo, THF (30 mL) and NEt₃ (100 μL, 0.72 mmol) were added, and the mixture heated at reflux for 24 h. Although the reaction was still incomplete, the solvent was evaporated in vacuo, and the residue purified by column chromatography upon silica gel using hexane-Et₂O (98:2) as eluant to give N-[(1,4-dichloro-7-isoquinolinyl)methyl]-N-methyl-DL-phenylglycine t-butyl ester (120 mg, 0.28 mmol) as a colourless oil.

The corresponding dihydrochloride salt was prepared as follows: a solution of the amine in hexane was stirred with a solution of HCl in Et2O (0.5 M). The resulting white precipitate was collected by filtration and dried.

mp 120-122° C.

¹H (CDCl₃, 400 MHz) δ 1.5 (9H, s), 2.25 (3H, s), 3.8 (1H, d), 3.9 (1H, d), 4.3 (1H, s), 7.3-7.4 (3H, m), 7.45-7.5 (2H, m), 7.95 (1H, d), 8.15 (1H, d), 8.2 (1H, s), 8.3 (1H, s) ppm.

LRMS 432 (MH⁺).

Anal. Found: C, 56.62; H, 5.58; N, 5.63. Calc for C₂₃H₂₄Cl₂N₂O₂HClH₂O: C, 56.86; H, 5.60; N, 5.77.

### Preparation 81

### N-Benzyl-N-[(1,4-dichloro-7-isoquinolinyl)methyl]glycine t-butyl ester

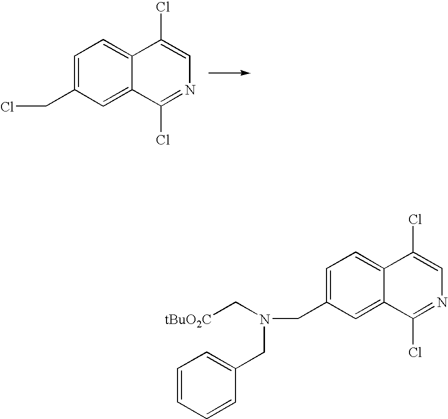

7-(Chloromethyl)-1,4-dichloroisoquinoline (378 mg, 1.53 mmol) was added to a stirred solution of N-benzyl glycine t-butyl ester (340 mg, 1.53 mmol) and NEt₃ (256 μL, 1.84 mmol) in THF (20 mL) and the mixture heated at reflux for 18 h. The solvent was evaporated in vacuo and the residue was purified by column chromatography upon silica gel using hexane-EtOAc (95:5 to 90:10) as eluant to give N-benzyl-N-[(1,4-dichloro-7-isoquinolinyl)methyl]glycine t-butyl ester (245 mg, 0.57 mmol).

The corresponding dihydrochloride salt was prepared as follows: a solution of the amine in Et₂O was stirred with a solution of HCl in dioxane (0.5 M). The resulting white precipitate was collected by filtration and dried.

mp 140-143° C.

¹H (CDCl₃, 400 MHz) δ 1.4 (9H, s), 3.3 (2H, s), 4.6 (2H, s), 4.8 (2H, s), 7.4-7.45 (3H, m), 7.75-7.8 (2H, m), 8.35 (1H, d), 8.4 (1H, s), 8.45 (1H, s), 8.8 (1H, d) ppm.

LRMS 433 (MH⁺).

Anal. Found: C, 58.91; H, 5.38; N, 5.90. Calc for C₂₃H₂₄Cl₂N₂O₂.HCl: C, 59.05; H, 5.39; N, 5.99.

### Preparation 82

### Nα-[(1,4-Dichloro-7-isoquinolinyl)sulphonyl]-Nε-tert-butyloxycarbonyl-L-lysine tert-butyl ester

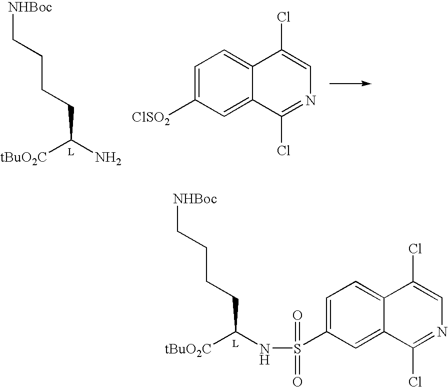

A solution of 1,4-dichloro-7-isoquinolinylsulphonyl chloride (250 mg, 0.84 mmol), Nε-tert-butyloxycarbonyl-L-lysine tert-butyl ester hydrochloride (286 mg, 0.84 mmol) and triethylamine (235 μl, 1.69 mmol) in CH₂Cl₂ (25 ml) was stirred at 23° C. for 3 h. The reaction mixture was washed with water (2×20 ml), dried (MgSO₄) and concentrated in vacuo to a residue which upon trituration with hexane and then i-Pr₂O gave Nα-[(1,4-dichloro-7-isoquinolinyl)sulphonyl]-Nε-tert-butyloxycarbonyl-L-lysine tert-butyl ester as a white powder (270 mg, 0.48 mmol).

¹H (CDCl₃, 300 MHz) δ 1.1 (9H, s), 1.35-1.5 (13H, m), 1.6-1.85 (2H, m), 3.0-3.2 (2H, m), 3.8-3.95 (1H, m), 4.45-4.6 (1H, br m), 5.35 (1H, d), 8.2 (1H, dd), 8.35 (1H, d), 8.45 (1H, s), 8.8 (1H, d) ppm.

LRMS 562 (MH⁺), 584 (MNa⁺).

Anal. Found: C, 51.04; H, 5.96; N, 7.42. Calc for C₂₄H₃₃Cl₂N₃O₆S: C, 51.24; H, 5.91; N, 7.47.

### Preparation 83

### Nα-[(1,4-Dichloro-7-isoquinolinyl)sulphonyl]-Nε-tert-butyloxycarbonyl-D-lysine tert-butyl ester

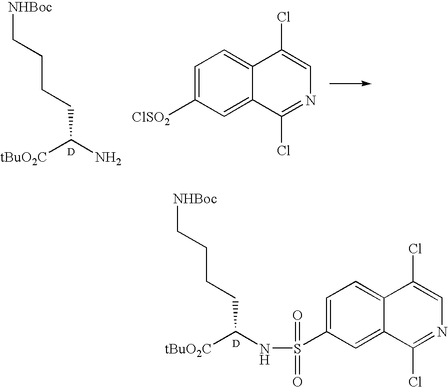

A solution of 1,4-dichloro-7-isoquinolinylsulphonyl chloride (250 mg, 0.84 mmol), Nε-tert-butyloxycarbonyl-D-lysine tert-butyl ester hydrochloride (286 mg, 0.84 mmol) and triethylamine (235 ηl, 1.69 mmol) in CH₂Cl₂ (25 ml) was stirred at 23° C. for 18 h. The reaction mixture was concentrated in vacuo and the residue purified by column chromatography upon silica gel using hexane-EtOAc (70:30) as eluant. Crystallisation from i-Pr₂O gave Nα-[(1,4-dichloro-7-isoquinolinyl)sulphonyl]-NΕ-t-butyloxycarbonyl-D-lysine tert-butyl ester (285 mg, 0.51 mmol).

¹H (CDCl₃, 400 MHz) δ 1.15 (9H, s), 1.2-1.55 (13H, m), 1.55-1.8 (2H, m), 3.05-3.15 (2H, m), 3.85-3.9 (1H, m), 4.5-4.6 (1H, m), 5.4 (1H, br d), 8.2 (1H, d), 8.35 (1H, d), 8.45 (1H, s), 8.8 (1H, s) ppm.

LRMS 584 (MNa⁺).

Anal. Found: C, 51.18; H, 5.89; N, 7.33. Calc for C₂₄H₃₃Cl₂N₃O₆S: C, 51.24; H, 5.91; N, 7.47.

### Preparation 84

### N-[(1,4-Dichloro-7-isoquinolinyl)sulphonyl]-L-glutamine tert-butyl ester

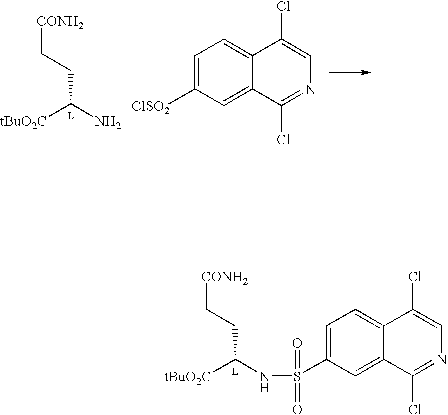

A solution of 1,4-dichloro-7-isoquinolinyl sulphonylchloride (250 mg, 0.84 mmol), L-glutamine tert-butyl ester hydrochloride (201 mg, 0.84 mmol) and triethylamine (235 μl , 1.69 mmol) in CH₂Cl₂ (25 ml) was stirred at 23° C. for 18 h. The reaction mixture was washed with water (2×20 ml) and the solvent removed in vacuo to give N-[(1,4-dichloro-7-isoquinolinyl)sulphonyl]-L-glutamine tert-butyl ester (309 mg, 0.67 mmol). An analytical sample was obtained following crystallisation from EtOAc.

¹H (CDCl₃, 300 MHz) δ 1.05-1.15 (9H, s), 1.8-1.95 (1H, m), 2.1-2.25 (1H, m), 2.35-2.55 (2H, m), 3.9-4.0 (1H, m), 5.4-5.6 (1H, br s), 5.6-5.8 (1H, br s), 5.85 (1H, d), 8.2 (1H, d), 8.35 (1H, d), 8.5 (1H, s), 8.8 (1H, s) ppm.

LRMS 462 (MH⁺), 479 (MNH₄⁺).

Anal. Found: C, 46.66; H, 4.54; N, 8.96. Calc for C₁₉H₂₁Cl₂N₃O₅S: C, 46.75; H, 4.58; N, 9.09.

### Preparation 85

### N-[(1,4-Dichloro-7-isoquinolinyl)sulphonyl]-cyclopentylamin

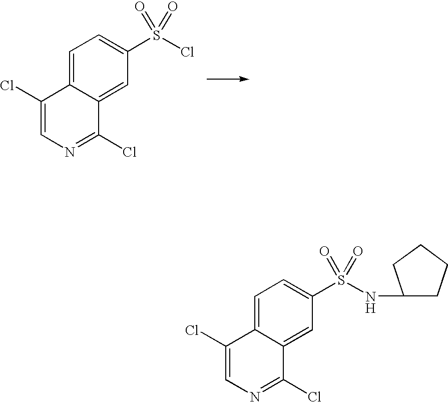

1,4-Dichloro-7-isoquinolinylsulphonyl chloride (250 mg, 0.84 mmol) was added to a solution of cyclopentylamine (100 μl, 1.0 mmol) and triethylamine (170 μl, 1.22 mmol) in CH₂Cl₂ (15 mL), and the reaction stirred at room temperature for 18 h. The solution was diluted with CH₂Cl₂, washed with 2M hydrochloric acid, saturated aqueous Na₂CO₃ solution and then brine. This solution was dried (MgSO₄), and evaporated in vacuo, to give N-[(1,4-dichloro7-isoquinolinyl)sulphonyl]-cyclopentylamine (250 mg, 0.72 mmol) as a white crystalline solid.

¹H (CDCl₃, 300 MHz) δ 1.4 (2H, m), 1.5-1.7 (4H, m), 1.85 (2H, m), 3.75 (1H, m), 4.6 (1H, d), 8.25 (1H, d), 8.4 (1H, d), 8.5 (1H, s), 8.95 (1H, s) ppm.

LRMS 346 (MH⁺)

Anal. Found: C, 48.68; H, 4.02; N, 7.97. Calc. for C₁₄H₁₄C₁₂N₂O₂S: C, 48.71; H, 4.09; N, 8.11%,

### Preparation 86

### 1,4-Dichloro-7-(1-pyrrolidinylsulphonyl)isoquinoline

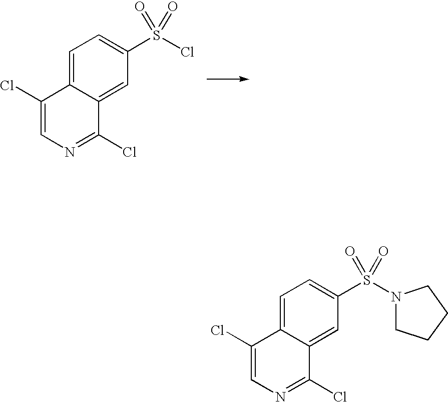

Pyrrolidine (96 mg, 1.35 mmol) was added to a solution of 1,4-dichloro-7-isoquinolinylsulphonyl chloride (20 mg, 0.67 mmol) in CH₂Cl₂ (5 ml), and the reaction stirred at room temperature for 72 h. The mixture was concentrated in vacuo, and the residual solid triturated with water, filtered and dried. The crude product was purified by column chromatography upon silica gel using EtOAc-hexane (50:50) as eluant, and recrystallised from i-Pr₂O, to give 1,4-dichloro-7-(1-pyrrolidinylsulphonyl)isoquinoline (67 mg, 0.20 mmol) as a white solid,

¹H (CDCl₃, 300MHz) δ 1.8 (4H, m), 3.35 (4H, m), 8.25 (1H, d), 8.4 (1H, d), 8.5 (1H, s), 8.85 (1H, s) ppm.

LRMS: 331, 333 (MH⁺)

Anal. Found: C, 47.23; H, 3.60; N, 8.32. Calc. for C₁₃H₁₂N₂Cl₂O₂S: C, 47.14; H, 3.65; N, 8.46%

### Preparation 87

### tert-Butyl (2R)-1-[(1,4-dichloro-7-isoquinolinyl)sulphonyl]-2-piperidinecarboxylate

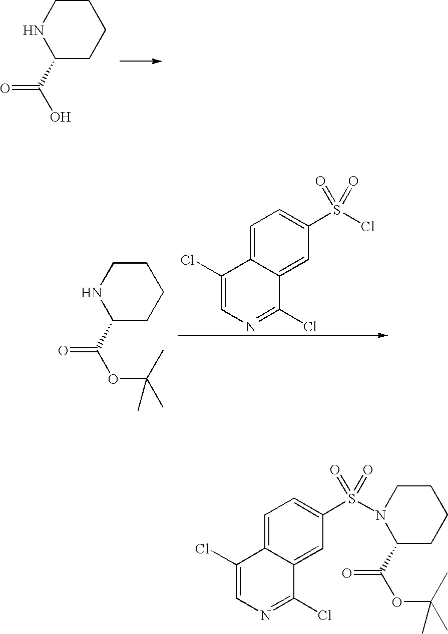

Concentrated H₂SO₄ (2.0 ml) was added to an ice-cold solution of 2-(R)-piperidine carboxylic acid (415 mg, 3.21 mmol) in dioxan (10 ml). Condensed isobutylene (40 ml) was carefully added, and the reaction stirred at room temperature in a sealed vessel for 21 h. The reaction mixture was poured into an ice-cooled solution of Et₂O (100 ml) and 5N NaOH (20 ml), the mixture allowed to warm to room temperature with stirring, and then diluted with water. The phases were separated, the organic layer washed with 1N NaOH, then concentrated in vacuo, to half the volume, and extracted with 2N HCl. The combined acidic extracts were basified using IN NaOH, and extracted with CH₂Cl₂, the combined organic solutions dried (MgSO₄) and evaporated in vacuo to afford tert-butyl 2(R)-piperidine carboxylate (210 mg, 1.14 mmol) as an oil.

¹H (CDCl₃, 300 MHz) δ 1.4-1.6 (11H, m), 1.75 (3H, m), 1.9 (1H, m), 2.65 (1H, m), 3.1 (1H, m), 3.2 (1H, m) ppm.

LRMS 186 (MH⁺).

1,4-Dichloro-7-isoquinolinylsulphonyl chloride (245 mg, 0.83 mmol) was added to a solution of tert-butyl 2(R)-piperidine carboxylate (153 mg, 0.83 mmol) and triethylamine (170 μl, 1.22 mmol) in CH₂Cl₂ (15 ml), and the reaction stirred at room temperature for 18 h. The solution was diluted with CH₂Cl₂, washed with 2M hydrochloric acid, saturated Na₂CO₃ solution and then brine, dried (MgSO₄), and evaporated in vacuo. The residual oil was purified by column chromatography upon silica gel using an elution gradient of pentane-EtOAc (100:0 to 90:10), to give tert-butyl (2R)-1-[(1,4-dichloro-7-isoquinolinyl)sulphonyl]-2-piperidinecarboxylate, (290 mg, 0.65 mmol) as a colourless film.

¹H (CDCl₃, 400 MHz) δ 1.3 (9H, s), 1.55 (2H, m), 1.7-1.85 (3H, m), 2.2 (1H, m), 3.3 (1H, dd), 3.9 (1H, dd), 4.75 (1H, d), 8.15 (1H, d), 8.35(1H, dd), 8.45 (1H, s), 8.8 (1H, s) ppm.

LRMS 462,464 (MNH₄⁺)

Anal. Found: C, 50.99; H, 4.95; N, 6.10. Calc. For C₁₉H₂₂Cl₂N₂O₄S; C, 51.24; H, 4.98; N, 6.29%.

### Preparation 88

### Methyl 4-{[(1,4-dichloro-7-isoquinolinyl)sulphonyl]amino}-1-methyl4-piperidinecarboxylate

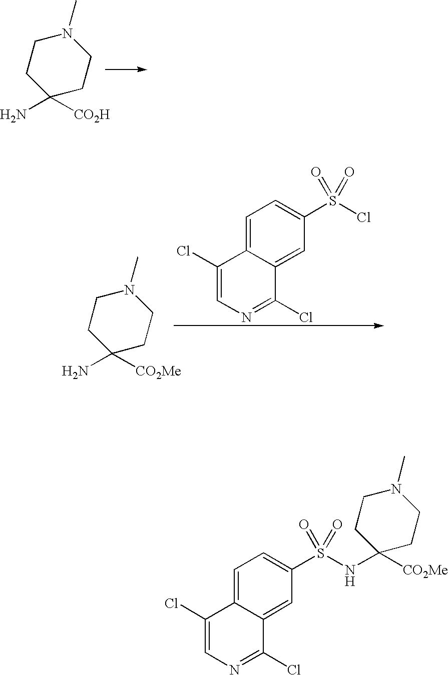

A solution of 4-amino-1-methyl-4-piperidinecarboxylic acid (4.0 g, 15.6 mmol) in methanolic HCl (100 ml) was stirred under reflux for 20 h. The cooled mixture was concentrated in vacuo and azeotroped with CH₂Cl₂ to give an oil. This was dissolved in ice-cold Na₂CO₃ solution and extracted with CH₂Cl₂ (2×). The combined organic extracts were dried (MgSO₄) and evaporated in vacuo to afford 4-amino-1-methyl-4-piperidinecarboxylate (1.6 g, 9.3 mmol) as an oil.

¹H (CDCl₃, 400 MHz) δ 1.4-1.65 (4H, m), 2.1-2.25 (2H, m), 2.35 (3H, s), 2.4-2.55 (4H, m), 3.75 (3H, s) ppm.

LRMS 173 (MH⁺)

1,4-Dichloro-7-isoquinolinylsulphonyl chloride (1.0 g, 3.37 mmol) was added to a solution of methyl 4-amino-1-methyl-4-piperidinecarboxylate (700 mg, 4.0 mmol) and triethylamine (700 μl, 1.0 mmol) in CH₂Cl₂ (60 ml), and the reaction stirred at room temperature for 18 h. The mixture was concentrated in vacuo, and the residue purified by column chromatography upon silica gel using an elution gradient of CH₂Cl₂-MeOH-0.880 NH₃ (97:3:0.3 to 95:5:0.5) to give methyl 4-{[(1,4-dichloro-7-isoquinolinyl)sulphonyl]amino}-1-methyl-4-piperidinecarboxylate (700 mg, 1.62 mmol) as a white solid.

¹H (CDCl₃, 400 MHz) δ 2.05 (2H, m), 2.25 (6H, m), 2.4 (2H, m), 2.55 (2H, m), 3.5 (3H, s), 8.25 (1H, d), 8.4 (1H, d), 8.5 (1H, s), 8.85 (1H, s) ppm.

LRMS 432, 434 (MH⁺)

### Preparation 89

### N-[(1,4-Dichloro-7-isoquinolinyl)sulphonyl]-N-(methyl)cycloleucine ethyl ester

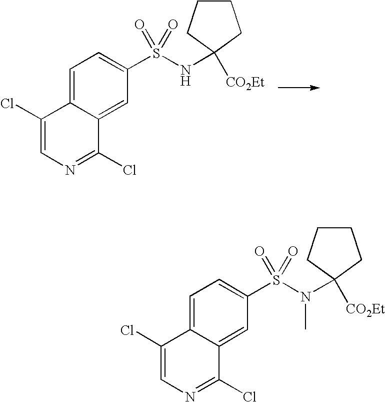

K₂CO₃ (238 mg, 1.73 mmol) was added to a solution of N-[(1,4-dichloro-7-isoquinolinyl)sulphonyl]-cycloleucine ethyl ester (300 mg, 0.72 mmol) in DMF (5 ml), and the mixture stirred at room temperature for 40 min. Methyl iodide (47 μl, 0.76 mmol) was added and the reaction stirred for a further 30 min. at room temperature. The mixture was poured into water, extracted with EtOAc, and the combined organic extracts washed with water, then brine, dried (Na₂SO₄) and evaporated in vacuo. The residual yellow solid was purified by column chromatography upon silica gel using EtOAc-hexane (20:80) as eluant to give N-[(1,4-dichloro-7-isoquinolinyl)sulphonyl]-N-(methyl)cycloleucine ethyl ester (204 mg, 0.47 mmol) as a white solid.

¹H (CDCl₃, 400 MHz) δ 1.25 (3H, t), 1.75 (4H, m), 2.1 (2H, m), 2.4 (2H, m) 3.05 (3H, s), 4.2 (2H, q), 8.25 (1H, d), 8.35 (1H, d), 8.5 (1H, s), 8.9 (1H, s) ppm.

LRMS 431, 433 (MH⁺)

Anal. Found: C, 50.12; H, 4.66; N, 6.43. Calc. for C₁₈H₂₀Cl₂N₂O₄S: C, 50.12; H, 4.67; N, 6.49%.

### Preparation 90

### 4-Bromo-1-chloro-7-isoquinolinesulphonyl chloride

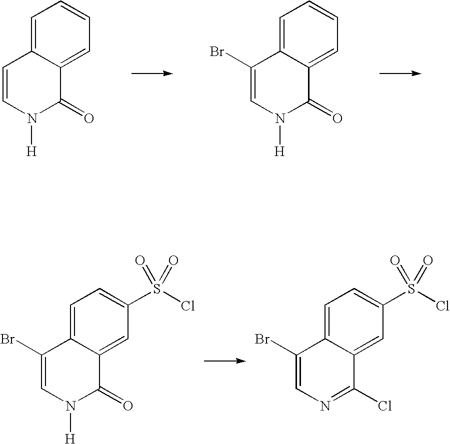

A suspension of isoquinolinol (10 g, 68.9 mmol) in MeCN (250 ml) at 50° C., was treated with N-bromosuccinimide (12.6 g, 70.8 mmol) whereupon almost complete solution occurred before a thick white precipitate was formed. After heating under reflux for 3 h, the reaction mixture was cooled in ice and the solid filtered, washed with MeCN, and dried to afford 4-bromo-1-(2H)-isoquinolone (7.6 g, 34.0 mmol).

¹H (DMSO-d₆, 300 MHz) δ 7.55 (1H, s), 7.6 (1H, m), 7.75 (1H, d), 7.85 (1H, m), 8.2 (1H, d), 11.55 (1H, br s) ppm.

LRMS 223, 225 (MH⁺).

4-Bromo-1-(2H)-isoquinolone (7.5 g, 33.0 mmol) was added portionwise to chlorosulphonic acid (23 ml, 346 mmol) and the resultant solution heated to 100° C. for 2½ days. After cooling, the reaction mixture was poured carefully onto ice to give a white solid which was filtered, washed with water, MeCN, and Et₂O and air-dried to give a cream solid. 4-Bromo-1-oxo-1,2-dihydro-7-isoquinolinesulphonyl chloride (13.5 g) was immediately used without further drying.

mp >300° C.

¹H (DMSO-d₆, MHz) δ 7.45 (1H, s), 7.7 (1H, d), 8.0 (1H, d), 8.45 (1H, s), 11.55 (1H, br s) ppm.

To a stirred solution of 4-bromo-1-oxo-1,2-dihydro-7-isoquinolinesulphonyl chloride (−13.5 g) in acetonitrile (200 ml) was added portionwise POCl₃ (10 ml, 110 mmol). The resultant heterogeneous mixture was heated under reflux for 24 h, allowed to cool, and the supernatant decanted from the brown oily residues and concentrated to a solid. Extraction of the solid into EtOAc gave, after solvent removal, a sticky solid which was triturated with Et₂O to afford the title compound (3.83 g, 11.0 mmol) as a white solid.

mp 120.5-121° C.

¹H (DMSO-d₆, 300 MHz) δ 8.2 (2H, m), 8.5 (1H, s), 8.6 (1H, s) ppm.

Anal. Found: C, 31.21; H, 1.27; N, 4.08. Calc for C₉H₄BrCl₂NO₂S.0.25H₂O: C, 31.29; H, 1.31; N, 4.05.

### Preparation 91

### N-[(4-Bromo-1-chloro-7-isoquinolinyl)sulphonyl]-D-proline tert-butyl ester

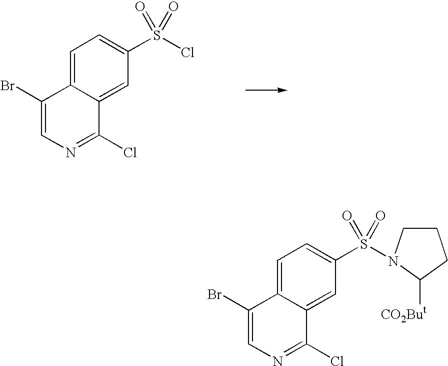

4-Bromo-1-chloro-7-isoquinolinesulphonyl chloride (400 mg, 1.17 mmol) in CH₂Cl₂ (20 ml) was treated with (D)-proline tert-butyl ester hydrochloride (250 mg, 1.20 mmol) and triethylamine (410 μl, 2.94 mmol) and stirred at room temperature for 2 h. The reaction was diluted with CH₂Cl₂, washed consecutively with water, 10% aqueous citric acid and brine, and then dried (MgSO₄) and concentrated in vacuo to give an off-white solid.

This was purified by column chromatography upon silica gel eluting with EtOAc-hexane (16:84) to give N-[(4-bromo-1-chloro-7-isoquinolinyl)sulphonyl]-D-proline tert-butyl ester (350 mg, 0.74 mmol) as a white solid.

mp 128.5-129.5° C.

¹H (CDCl₃, 300 MHz) δ 1.1 (9H, s), 1.85-2.0 (3H, m), 2.2 (1H, m), 3.5 (2H, m), 4.4 (1H, dd), 8.3 (2H, m), 8.6 (1H, s), 8.9 (1H, s) ppm.

LRMS 475, 477 (MH⁺).

Anal. Found: C, 45.41; H, 4.21; N, 5.83. Calc for C₁₈H₂₀BrClN₂O₄S: C, 45.44; H, 4.24; N, 5.89.

### Preparation 92

### N-{[(4-Bromo-1-chloro-7-isoquinolinyl)sulphonyl]-N-[2-(dimethylamino)ethyl]cycloleucine ethyl ester hydrochloride

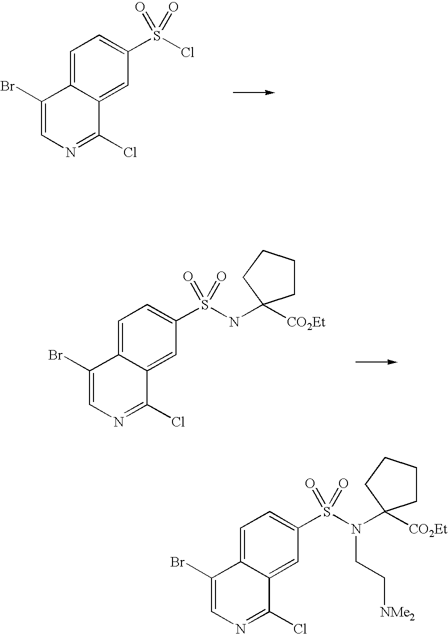

Triethylamine (1.02 ml, 7.33 mmol) was added to a solution of 4-bromo-1-chloroisoquinolinylsulphonyl chloride (1.0 g, 2.93 mmol) in CH₂Cl₂ (25 ml) and the reaction stirred at room temperature for 2 h. The reaction was washed consecutively with 1N HCl, Na₂CO₃ solution, and brine, then dried (Na₂SO₄) and evaporated in vacuo. The residual oil was crystallised from CH₂Cl₂-i-Pr₂O to give N-{((4-bromo-1-chloro-7-isoquinolinyl)sulphonyl]cycloleucine ethyl ester (380 mg, 0.82 mmol) as a solid.

¹H (CDCl₃, 300 MHz) δ 1.2 (3H, t), 1.6-1.8 (4H, m), 2.0 (2H, m), 2.15 (2H, m), 4.05 (2H, q), 8.25 (1H, d), 8.35 (1H, d), 8.6 (1H, s), 8.9 (1H, s) ppm.

LRMS 484 (MNa⁺)

K₂CO₃ (157 mg, 1.14 mmol) was added to a solution of N-{[(4-bromo-1-chloro-7-isoquinolinyl)sulphonyl]cycloleucine ethyl ester (300 mg, 0.65 mmol) in DMF (5 ml), and the solution stirred for 5 min. N,N-dimethylaminoethyl chloride hydrochloride (112 mg, 0.78 mmol) was added and the reaction stirred at room temperature for 36 h. The reaction mixture was partitioned between water and EtOAc, the layers separated, and the aqueous phase extracted with EtOAc. The combined organic solutions were washed with brine, dried (Na₂SO₄) and evaporated in vacuo. The residue was purified by column chromatography upon silica gel using CH₂Cl₂-MeOH-0.880 NH₃ (95:5:0.5) as eluant, to give a gum. This was dissolved in an Et₂O-EtOAc solution, ethereal HCl added and the mixture evaporated in vacuo. The resulting solid was triturated with water, filtered and dried to give N-([(4-bromo-1-chloro-7-isoquinolinyl)sulphonyl]-N-[2-(dimethylamino)ethyl]cycloleucine ethyl ester hydrochloride (90 mg, 0.16 mmol) as a solid.

¹H (CDCl₃, 300 MHz) δ 1.3 (3H, t), 1.65 (2H, m), 1.8 (2H, m), 2.15 (2H, m), 2.4 (2H, m), 2.9 (6H, m), 3.6 (2H, m), 4.0 (2H, m), 4.2 (2H, q), 8.2 (1H, d), 8.4 (1H, d), 8.65 (1H, s), 8.80 (1H, s) ppm.

LRMS 534 (MH⁺)

Anal Found: C, 44.17; H, 4.97; N, 7.24. Calc. for C₂₁H₂₇BrClN₃OS.HCl: C, 44.30; H, 4.96; N, 7.38%.

### Preparation 93

### Ethyl 3-{[(1,4-dichloro-7-isoquinolinyl)sulphonyl]amino}-2,2-dimethylpropanoate hydrochloride

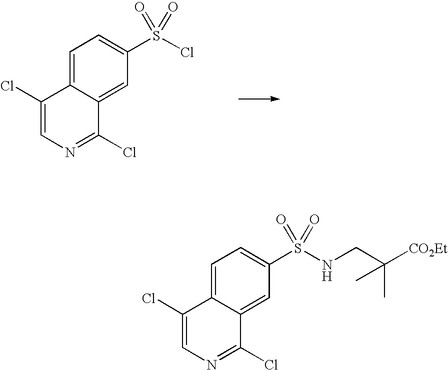

The title compound was obtained as a white solid (86%) from 1,4-dichlorosulphonyl chloride and ethyl 3-amino-2,2-dimethylpropanoate hydrochloride, following a similar procedure to that described in preparation 90.

¹H (CDCl₃, 300 MHz) δ 1.25 (9H, m), 3.0 (2H, d), 4.1 (2H, q), 5.4 (1H, t), 8.2 (1H, d), 8.4 (1H, d), 8.5 (1H, s), 8.9 (1H, s) ppm.

LRMS 404, 406 (MH⁺)

Anal. found: C, 47.39; H, 4.44: N, 6.73. Calc. for C₁₆H₁₈Cl₂N₂O₄S: C, 47.42; H, 4.48; N, 6.91%.

### Preparation 94

### N-[(1,4-Dichloro-7-isoquinolinyl)sulphonyl]-N-(2-(tetrahydro-2H-pyran-2-yloxy)ethyl]cycloleucine ethyl ester

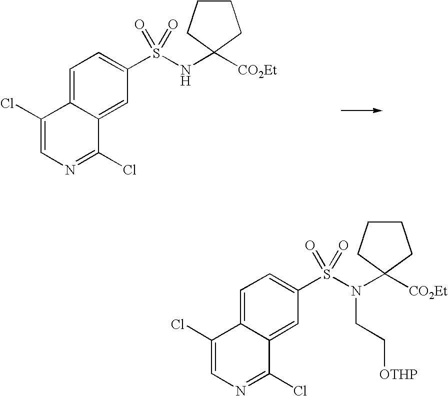

K₂CO₃ (238 mg, 1.73 mmol) was added to a solution of N-[(1,4-dichloro-7-isoquinolinyl)sulphonyl]cycloleucine ethyl ester (600 mg, 1.44 mmol) in DMF (10 ml), and the suspension stirred at room temperature for 30 min. A solution of 2-(2-bromoethoxy)tetrahydro-2H-pyran (J.C.S. 1948; 4187) (316 mg, 1.44 mmol) in DMF (4 ml) was added, followed by sodium iodide (10 mg), and the reaction stirred at 70° C. for 23 h. The cooled mixture was poured into water, and extracted with EtOAc. The combined organic extracts were washed with brine, dried (MgSO₄), and evaporated in vacuo. The residual yellow oil was purified by column chromatography upon silica gel using hexane-Et₂O (75:25) as eluant, azeotroped with CH₂Cl₂ and dried under vacuum to afford N-[(1,4-dichloro-7-isoquinolinyl)suphonyl]-N-[2-(tetrahydro-2H-pyran-2-yloxy)ethyl]cycloleucine ethyl ester (341 mg, 0.63 mmol) as a solid.

¹H (CDCl₃, 400 MHz) δ 1.3 (3H, t), 1.55 (4H, m), 1.65-1.8 (6H, m), 2.15 (2H, m), 2.4 (2H, m), 3.5 (1H, m), 3.7 (3H, m), 3.8 (1H, m), 3.95 (1H, m), 4.2 (2H, q), 4.55 (1H, m), 8.35 (2H, s), 8.45 (1H, s), 8.9 (1H, s) ppm.

LRMS 545 (MH⁺), 562 (MNH₄⁺)

Anal. Found: C, 52.3 1; H, 5.58; N, 4.84. Calc. for C₂₄H₃₀Cl₂N₂O₆S.0.3H₂O: C, 52.33; H, 5.60; N, 5.09%.

### Preparation 95

### N-[(1,4-dichloro-7-isoquinolinyl)methyl]cycloleucine methyl ester

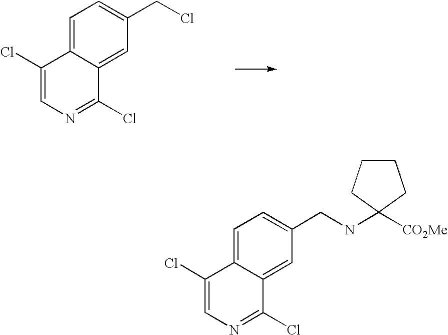

7-Chloromethyl-1,4-dichloro-isoquinoline (400 mg, 1.62 mmol) was added to a suspension of cycloleucine methyl ester (255 mg, 1.78 mmol), K₂CO₃ (500 mg, 3.62 mmol) and sodium iodide (15 mg) and the resultant mixture heated to 75° C. for 2½ h. After cooling, the reaction mixture was poured into water and extracted with CH₂Cl₂ (2×60 ml). The organic extracts were washed with water, brine, dried (Na₂SO₄) and concentrated in vacuo to give an oil. This was purified by column chromatography upon silica gel eluting with hexane-EtOAc (85 : 15) to give N-[(1,4-dichloro-7-isoquinolinyl)methyl]cycloleucine methyl ester (414 mg, 1.17 mmol) as a yellow oil.

A sample of this oil was treated with ethereal HCl, and the mixture evaporated to give the hydrochloride salt of the title compound as a white solid.

¹H (CDCl₃, 300 MHz) δ 1.4-1.8 (5H, m), 2.0 (3H, m), 3.75 (3H, s), 4.15 (2H, s), 8.25 (3H, m), 8.5 (1H, s), 10.5 (2H, br s) ppm.

Anal. found: C, 52.53; H. 4.99; N, 6.84. Calc. for C₁₇H₁₉Cl₂N₂O₂.HCl: C, 52.39; H, 4.91; N, 7.19%.

### Preparation 96

### (1-Aminocyclopentyl)(4-methyl-1-piperazinyl)methanone dihydrochloride

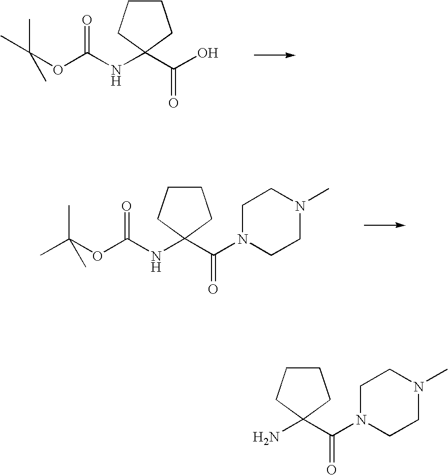

1-(3-Dimethylaminopropyl)-3-ethylcarbodiimide hydrochloride (2.49 g, 13.0 mmol) was added portionwise to a cooled (4CC) solution of hydroxybenzotriazole hydrate (1.49 g, 11.0 mmol) and 1-[(tert-butoxycarbonyl)amino]cyclopentanecarboxylic acid (2.29 g, 10.0 mmol) in DMF (15 ml), and the mixture stirred for 30 min. N-Methylpiperazine (1.10 g, 11.0 mmol) was added, the reaction stirred for 30 min. allowed to warm to room temperature and stirring continued for a further 17 h. The reaction mixture was evaporated in vacuo, and the residual yellow oil partitioned between saturated Na₂CO₃ solution and EtOAc. The layers were separated, the aqueous phase extracted with EtOAc, and the combined organic solutions dried (MgSO₄) and concentrated in vacuo. The residual solid was pre-adsorbed onto silica gel and purified by column chromatography upon silica gel using an elution gradient of CH₂Cl₂-MeOH-0.880 NH₃ (97.5:2.5:0.25 to 90:10: 1) and triturated with Et₂O to afford tert-butyl 1-[(4-methyl-1-piperazinyl)carbonyl]cyclopentylcarbamate (2.31 g, 7.4 mmol) as a crystalline solid.

mp 171-175° C.

¹H (CDCl₃, 300 MHz) δ 1.4 (9H, s), 1.7 (6H, m), 2.25 (3H, s), 2.4 (6H, m), 3.65 (4H, m), 4.7 (1H, br s).

LRMS 312 (MH⁺)

A suspension of tert-butyl 1-[(4-methyl-1-piperazinyl)carbonyl]cyclopentylcarbamate (2.2 g, 7.06 mmol) in EtOAc (120 ml) at 4° C. was saturated with HCl gas, and the reaction then stirred for 4 h. The mixture was azeotroped with EtOAc, then dry Et₂O, and dried under vacuum to afford (1-aminocyclopentyl)(4-methyl-1-piperazinyl)methanone dihydrochloride (2.1 g) as a white solid.

mp 267-270° C. (Decomp)

Anal. Found: C, 43.29; H, 7.99; N, 13.84. Calc. for C₁₁H₂₁N₃O.2HCl.H₂O: C, 43.71; H, 8.34; N. 13.90%.

LRMS 212 (MH⁺)

PCS9482 Compounds

As indicated above, suitable inhibitor compounds (agents) for use in the present invention are disclosed in GB patent application No. 9908410.5 (incorporated herein by reference) and in U.S. patent application Ser. No. 09/546,410 (incorporated herein by reference) and European patent application No. 00302778.6 (incorporated herein by reference) and in Japanese patent application No. 2000-104725 (incorporated herein by reference). It is to be understood that if the following teachings refer to further statements of inventions and preferred aspects then those statements and preferred aspects have to be read in conjunction with the aforementioned statements and preferred aspects—viz pharmaceutical compositions either comprising an iUPA and/or an iMMP and a growth factor (as well as the uses thereof) or comprising an iUPA and an iMMP and an optional growth factor (as well as the uses thereof).

The PCS9482 compounds are pyridine derivatives useful as urokinase inhibitors, and in particular to 2-diaminomethyleneaminopyridine derivatives, alternatively named as 2-pyridylguanidine derivatives, useful as urokinase inhibitors.

The PCS9482 compounds are of the general formula (1) 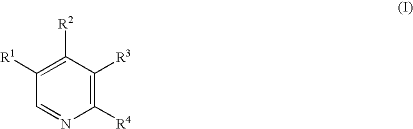
or a pharmaceutically acceptable salt thereof, or solvate of either entity,
wherein
R¹ is H, halogen, CN, C₁₋₆ alkyl optionally substituted by one or more halogen, or C₁₋₄ alkoxy optionally substituted by one or more halogen,
R² and R³ are each independently H, halogen, C₁₋₆ alkyl optionally substituted by one or more halogen or C₁₋₆ alkoxy, aryl, (Cₙ-alkylene)CO₂H, (Cₙ-alkylene)CO₂(C₁₋₆ alkyl), (Cₙ-alkylene)CONR⁵R⁶, CH═CHR⁷, CH═CHCO₂H, CH═CHCONR⁵R⁶, CH═CHSO₂NR⁵R⁶, C═CR⁷, O(Cₘ-alkylene)OH, O(Cₘ-alkylene)OR⁸, OR⁸, O(Cₘ-alkylene)CONR⁵R⁶, CH₂OR⁸ or CH₂NR⁵R⁶,
R⁴ is N═C(NH₂)₂ or NHC(═NH)NH₂,
R⁵ and R⁶ are each independently H, C₁₋₆ alkyl optionally substituted by OH or CO₂H, het(C₁₋₆ alkylene) or aryl(C₁₋₆ alkylene), or can be taken together with the nitrogen to which they are attached, to form a 4- to 7-membered saturated ring optionally containing an additional hetero-moiety selected from O, S or NR⁹,
and which ring is optionally benzo-fused,
and which optionally benzo-fused ring is optionally substituted by up to three substituents independently selected from OH, halogen, CO₂H, CO₂(C₁₋₆ alkyl) and C₁₋₆ alkyl,
R⁷ is C₁₋₆ alkyl, aryl or het;
R⁸ is C₁₋₆ alkyl, aryl, het, aryl(CHCO₂H) or aryl(C₁₋₆ alkylene);
R⁹ is H, C₁₋₆ alkyl, or CO(C₁₋₆ alkyl);
wherein “aryl”, including the aryl moiety of the aryl(C₁₋₆ alkylene) group, means phenyl optionally substituted by up to three substituents independently selected from halogen, C₁₋₆ alkyl, (Cₙ-alkylene)CO₂H, (Cₙ-alkylene)CO₂(C₁₋₆ alkyl), (Cₙ-alkylene)CN, C₁₋₆ alkoxy, CN, (Cₙ-alkylene)CONR⁵R⁶, CH═CHCO₂H, CH═CHCONR⁵R⁶, CH═CHSO₂NR⁵R⁶, O(Cₘ-alkylene)OH, CH₂NR⁵R⁶, and O(Cₘ-alkylene)CONR⁵R⁶;
“het” means an optionally benzo-fused 5- or 6-membered saturated or unsaturated heterocycle linked by any available atom in the heterocyclic or benzo-ring (if present), which heterocyclic group is selected from dioxolyl, furyl, thienyl, pyrrolyl, oxazolyl, thiazolyl, isoxazolyl, isothiazolyl, imidazolyl, pyrazolyl, oxadiazolyl, thiadiazolyl, triazolyl, tetrazolyl, pyridyl, pyrimidinyl, pyridazinyl, pyrazinyl, and pyranyl,
and which optionally benzo-fused heterocycle is optionally substituted by up to three substituents independently selected from halogen, C₁₋₆ alkyl, (Cₙ-alkylene)CO₂H, (Cₙ-alkylene)CO₂(C₁₋₆ alkyl), (Cₙ-alkylene)CN, (Cₙ-alkylene)CON⁵R⁶, CH═CHCO₂H, CH═CHCONR⁵R⁶, CH═CHSO₂NR⁵R⁶, O(Cₘ-alkylene)OH, CH₂NR⁵R⁶, and O(Cₘ-alkylene)CONR⁵R⁶;
n is 0, 1 or 2;
m is 1 or 2;
and wherein the “C-alkylene” linking groups in the definitions above are optionally substituted by one or more C₁₋₆ alkyl;
with the proviso that R¹, R² and R³ are not all H;
hereinafter referred to as “substances of the invention”.

“Alkyl” groups and the alkyl moiety of “alkoxy” groups can be straight-chain, branched or cyclic where the number of carbon atoms allows.

“Halogen” means F, Cl, Br or I.

The two definitions given for the R⁴ moiety are of course tautomeric. The skilled man will realise that in certain circumstances one tautomer will prevail, and in other circumstances a mixture of tautomers will be present.

Preferably R¹ is H, CN, halogen or methyl optionally substituted by one or more halogen.

More preferably R¹ is H, CN, Cl, Br or methyl.

Most preferably R¹ is Cl or Br.

Preferably R² is H, halogen, C₁₋₆ alkyl optionally substituted by one or more halogen, aryl, CH₂OR⁸, (Cₙ-alkylene)CONR⁵R⁶, CO₂H or CH₂NR⁵R⁶.

More preferably R² is H, Cl, methyl, phenyl, CONHCH₂Ph, CH₂OPh, CH₂NCH₃Bn, or pyrrolidinomethyl.

Most preferably R² is H.

Preferably R³ is H, Cl, Br, CF₃, aryl, (Cₙ-alkylene)CO₂H, (Cₙ-alkylene)CO₂(C₁₋₆ alkyl), (Cₙ-alkylene)CONR⁵R⁶, CH═CHR⁷, CH═CHCO₂H, CH═CHCONR⁵R⁶, CH═CHSO₂NR⁵R⁶, C═CR⁷, O(Cₘ-alkylene)OH, O(Cₘ-alkylene)OR⁸, OR⁸, O(Cₘ-alkylene)CONR⁵R⁶, CH₂OR⁸, or CH₂NR⁵R⁶.

More preferably R³ is CH═CHCO₂H, (2-carboxypyrrolidino)SO₂CH═CH, (cyanophenyl)CH═CH, or (carboxyphenyl)CH═CH.

Yet more preferably R³ is CH═CHCO₂H, (2-carboxypyrrolidino)SO₂CH═CH, (3-cyanophenyl)CH═CH, or (3-carboxyphenyl)CH═CH.

Most preferably R³ is (2-carboxypyrrolidino)SO₂CH═CH, (3-cyanophenyl)CH═CH, or (3-carboxyphenyl)CH═CH.

A preferable group of substances of the invention are those wherein R¹ is H, CN, Cl, Br or methyl; R² is H, Cl, methyl, phenyl, CONHCH₂Ph, CH₂OPh, CH₂NCH₃Bn, or pyrrolidinomethyl; and R³ is CH═CHCO₂H, (2-carboxypyrrolidino)SO₂CH═CH, (3-cyanophenyl)CH═CH, or (3-carboxyphenyl)CH═CH.

A yet more preferable group of substances of the invention are those in which R¹ is Cl or Br; R² is H; and R³ is (2-carboxypyrrolidino)SO₂CH═CH, (3-cyanophenyl)CH═CH, or (3-carboxyphenyl)CH═CH.

A further preferred group of substances of the invention are those mentioned below in the Examples and the salts and solvates thereof.

In the Synthetic Methods below, unless otherwise specified, the substituents are as defined above with reference to the compounds of formula (1) above.

Where desired or necessary the compound of formula (I) is converted into a pharmaceutically acceptable salt thereof. A pharmaceutically acceptable salt of a compound of formula (I) may be conveniently be prepared by mixing together solutions of a compound of formula (I) and the desired acid or base, as appropriate. The salt may be precipitated from solution and collected by filtration, or may be collected by other means such as by evaporation of the solvent.

Synthetic Methods

Method 1

Compounds of formula (I) can be obtained from the corresponding 2-aminopyridine derivative (II) by reaction with cyanamide (NH₂CN) or a reagent which acts as a “NHC⁺=NH” synthon such as carboxamidine derivatives, e.g. 1H-pyrazole-1-carboxamidine (M. S. Bernatowicz, Y. Wu, G. R. Matsueda, J. Org. Chem., 1992, 57, 2497), the 3,5-dimethylpyrazole analogue thereof (M. A. Brimble et al, J.Chem.Soc.Perkin Trans.1 (1990)311), simple O-alkylthiouronium salts or S-alkylisothiouronium salts such as 0-methylisothiourea (F. El-Fehail et al, J.Med.Chem. (1986), 29, 984), S-methylisothiouronium sulphate (S. Botros et al, J. Med. Chem. (1986)29,874; P. S. Chauhan et al, Ind. J. Chem., 1993, 32B, 858) or S-ethylisothiouronium bromide (M. L. Pedersen et al, J.Org.Chem. (1993) 58, 6966). Alternatively aminoiminomethanesulphinic acid, or aminoiminomethanesulphonic acid may be used (A. E. Miller et al, Synthesis (1986) 777; K. Kim et al, Tet.Lett. (1988) 29,3183).

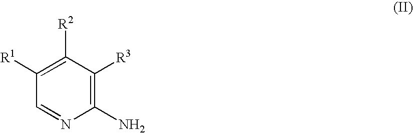

Other methods for this transformation are known to those skilled in the art (see for example, “Comprehensive Organic Functional Group Transformations”, 1995, Pergamon Press, Vol 6 p639, T. L. Gilchrist (Ed.); Patai's “Chemistry of Functional Groups”, Vol. 2. “The Chemistry of Amidines and Imidates”, 1991, 488).

2-Aminopyridines (II) may be prepared by standard published methods (see for example, “The Chemistry of Heterocyclic Compounds” Vol. 38 Pt. 2 John Wiley & Sons, Ed. F. G. Kathawala, G. M. Coppolq, H. F. Schuster) including, for example, by rearrangement from the corresponding carboxy-derivative (Hoffmann, Curtius, Lossen, Schmidt-type rearrangements) and subsequent deprotection.

Alternatively, 2-aminopyridines may be prepared by direct displacement of a ring hydrogen using the Chichibabin reaction (A. F. Pozharskii et. al. Russian Chem. Reviews, 1978, 47, 1042. C. K. McGill et. al. Advances in Heterocyclic Chemistry 1988, Vol. 44, 1)

2-Aminopyridines (II) may alternatively be prepared from the corresponding 2-halopyridines by direct displacement of a leaving group such as Cl or Br with a nitrogen nucleophile such as azide (followed by reduction), or by ammonia, or through Pd-catalysis with a suitable amine (such as benzylamine) followed by deprotection using standard conditions well-known in the art. Examples of such chemistry is outlined in “The Chemistry of Heterocyclic Compounds” Vol. 14, Pts. 2 and 3 John Wiley & Sons, in particular Pt. 2, (196 1), Pt. 3 (1962), Pt. 2-supplement (1974) and Pt. 3-supplement (1974

2-Halopyridines may be prepared by methods well known in the literature. For example, by treatment of 2-hydroxypyridines (2-pyrimidinones) with halogenating agents such as SOCl₂ (Y. S. Lo. Et. Al. Syn. Comm., 1988, 19, 553), POCl₃ (M. A. Walters, Syn. Comm., 1992, 22, 2829), or POBr₃ (G. J. Quallich, J. Org. Chem., 1992, 57, 761). Alternatively, 2-alkoxypyridines may be transformed to the corresponding 2-aminopyridines under Vilsmeir-Haack conditions such as POCl₃+DMF (L-L Lai et. Al. J. Chem. Res. (S), 1996, 194). The corresponding N-oxide may be treated with suitable halogenating reactions to directly produce 2-halopyridines—e.g. POCl₃/PCl₅ (M. A. Walters, Tetrahedron Lett., 1995, 42, 7575). Direct halogenation of the 2-position is possible in the presence of certain ring substituents (M. Tiecco et. al. Tetrahedron, 1986, 42, 1475, K. J. Edgar, J. Org Chem., 1990, 55, 5287).

Method 2

Compounds of formula (I) can be obtained from the corresponding 2-aminopyridine derivative (II) as defined in Method 1 above, via reaction with a reagent which acts as a protected amidine(2+) synthon 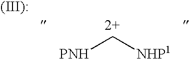
such as a compound PNHC(═Z)NHP¹, PN═CZ¹NHP¹ or PNHCZ¹═NP¹, where Z is a group such as O, or S and Z¹ is a leaving group such as Cl, Br, I, mesylate, tosylate, alkyloxy, etc., and where P and P¹ may be the same or different and are N-protecting groups such as are well-known in the art, such as t-butoxycarbonyl, benzyloxycarbonyl, arylsulphonyl such as toluenesulphonyl, nitro, etc.

Examples of reagents that act as synthons (III) include N,N′-protected-S-alkylthiouronium derivatives such as N,N′-bis(t-butoxycarbonyl)-S-Me-isothiourea, N,N′-bis(benzyloxycarbonyl)-S-methylisothiourea, or sulphonic acid derivatives of these (J. Org. Chem. 1986, 51, 1882), or S-arylthiouronium derivatives such as N,N′-bis(t-butoxycarbonyl)-S-(2,4-dinitrobenzene) (S. G. Lammin, B. L. Pedgrift, A. J. Ratcliffe, Tet. Lett. 1996, 37, 6815), or mono-protected analogues such as [(4-methoxy-2,3,6-trimethylphenyl)sulphonyl]-carbamimidothioic acid methyl ester or the corresponding 2,2,5,7,8-pentamethylchroman-6-sulphonyl analogue (D. R. Kent, W. L. Cody, A. M. Doherty, Tet. Lett., 1996, 37, 8711), or S-methyl-N-nitroisothiourea (L. Fishbein et al, J. Am. Chem. Soc. (1954) 76, 1877) or various substituted thioureas such as N,N′-bis(t-butoxycarbonyl)thiourea (C. Levallet, J. Lerpiniere, S. Y. Ko, Tet. 1997, 53, 5291) with or without the presence of a promoter such as a Mukaiyama's reagent (Yong, Y. F.; Kowalski, J. A.; Lipton, M. A. J. Org. Chem., 1997, 62, 1540), or copper, mercury or silver salts, particularly with mercury (II) chloride. Suitably N-protected O-alkylisoureas may also be used such as O-methyl-N-nitroisourea (N. Heyboer et al, Rec. Chim. Trav. Pays-Bas (1962)81,69). Alternatively other guanylation agents known to those skilled in the art such as 1-H-pyrazole-1-[N,N′-bis(t-butoxycarbonyl)]carboxamidine, the corresponding bis-Cbz derivative (M. S. Bernatowicz, Y. Wu, G. R. Matsueda, Tet. Lett. 1993, 34, 3389) or mono-Boc or mono-Cbz derivatives may be used (B. Drake. Synthesis, 1994, 579, M. S. Bernatowicz. Tet. Lett. 1993, 34, 3389). Similarly, 3,5-dimethyl-1-nitroguanylpyrazole may be used (T. Wakayima et al, Tet. Lett.( 1986)29,2143).

The reaction can conveniently be carried out using a suitable solvent such as dichloromethane, N,N-dimethylformamide (DMF), methanol.

The reaction is also conveniently carried out by adding mercury (II) chloride to a mixture of the aminopyridine (II) and a thiourea derivative of type (III) in a suitable base/solvent mixture such as triethylamine/dichloronmethane.

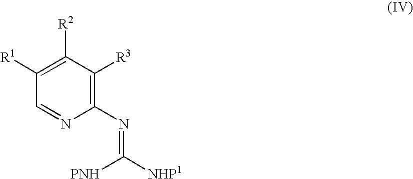

The product of this reaction is the protected pyridinylguanidine (IV), which can conveniently be deprotected to give (I) or a salt thereof. For example, if the protecting group P and/or P¹ is t-butoxycarbonyl, conveniently the deprotection is carried out using an acid such as trifluoroacetic acid (TFA) or hydrochloric acid, in a suitable solvent such as dichloromethane, to give a trifluoroacetate (triflate) salt of (I), either as the mono- or ditriflate.

If P and/or P¹ is a hydrogenolysable group, such as benzyloxycarbonyl, the deprotection could be performed by hydrogenolysis.

Other protection/deprotection regimes include:
nitro (K. Suzuki et al, Chem.Pharm.Bull. (1985)33,1528, Nencioni et al, J.Med.Chem.(1991)34,3373, B. T. Golding et al, J.C.S.Chem.Comm.(1994)2613;
p-toluenesulphonyl (J. F. Callaghan et al, Tetrahedron (1993) 49 3479;
mesitylsulphonyl (Shiori et al, Chem.Pharm.Bull.(1987)35,2698, ibid.(1987)35,2561, ibid., (1989)37,3432, ibid., (1987)35,3880, ibid., (1987)35,1076;
2-adamantoyloxycarbonyl (Iuchi et al, ibid., (1987) 35, 4307; and
methylsulphonylethoxycarbonyl (Filippov et al, Syn.Lett.(1994)922)

It will be apparent to those skilled in the art that other protection and subsequent deprotection regimes during synthesis of a compound of the invention may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

Method 3

Compounds with the formula (I) can be obtained from compounds of formula (V): 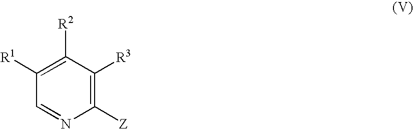
where Z is a suitable leaving group such as Cl, Br or OPh, by displacement of the leaving group by the free base of guanidine.

The free base of guanidine may conveniently be generated in situ from a suitable salt, such as the hydrochloride, carbonate, nitrate, or sulphate with a suitable base such as sodium hydride, potassium hydride, or another alkali metal base, preferably in a dry non-protic solvent such as tetrahydrofuran (THF), DMSO, N,N-dimethylformamide (DMF), ethylene glycol dimethyl ether (DME), N,N-dimethyl acetamide (DMA), toluene or mixtures thereof. Alternatively it can be generated from a suitable salt using an alkoxide in an alcohol solvent such as potassium t-butoxide in t-butanol, or in a non-protic solvent as above.

The thus formed free guanidine can be combined with the compound of formula (V) and the reaction to form compounds of formula (I) can be carried out at from room temperature to 200° C., preferably from about 50° C. to 150° C., preferably for between 4 hours and 6 days.

Method 4

Compounds of the formula (I) when one or more of R¹⁻³ contains a hydroxy group, may be prepared from a suitably “protected” hydroxy derivative, i.e. a compound of the formula (I) where one or more of R¹⁻³ contains a corresponding “OP²”, where P² is a suitable O-protecting group such as O-benzyl. The benzyl group may be removed for example by catalytic hydrogenation using a palladium on charcoal catalyst in a suitable solvent such as ethanol at about 20° C. and elevated pressure, optionally in the presence of an excess of an acid such as HCl or AcOH, or TFA, or by other known deprotection methods.

Suitable O-protecting groups and protection/deprotection can be found in the texts by Greene and Wuts, and Kocienski, supra.

Method 5

Compounds of the invention where R² or R³ is or contains a carboxylic acid group or carbamoyl group can be made from the corresponding compound where the substituent is or contains a nitrile by full or partial hydrolysis. Compounds of the invention where R² or R³ is or contains a carboxylic acid group can be made from the corresponding compound where the substituent is a carbamoyl moiety, by hydrolysis. The hydrolysis can be carried out by methods well-known in the art, for example those mentioned in “Advanced Organic Chemistry” by J. March, 3rd edition (Wiley-Interscience) chapter 6-5, and references therein. Conveniently the hydrolysis is carried out using concentrated hydrochloric acid, at elevated temperatures, and the product forms the hydrochloride salt.

Compounds of the formula (I) where one or more of R¹, R² or R³ is or contains Cl or Br may be dehalogenated to give the corresponding hydrido compounds of formula (I) by hydrogenolysis, suitably using a palladium on charcoal catalyst, in a suitable solvent such as ethanol at about 20° C. and at elevated pressure.

Compounds of formula (I) in which one or more of R² or R³ contains an amide moiety may be made via reaction of an optionally protected corresponding carboxy compound, by coupling with the amine of choice, e.g. via initial formation of the corresponding acid halide or mixed anhydride, and subsequent reaction with the amine, followed by deprotection if appropriate. Such transformations are well-known in the art.

Certain of the compounds of formula (I) which have an electrophilic group attached to an aromatic ring may be made by reaction of the corresponding hydrido compound with an electrophilic reagent. For example sulphonylation of the aromatic ring using standard reagents and methods, such as fuming sulphuric acid, gives a corresponding sulphonic acid. This can then be optionally converted into the corresponding sulphonamide by methods known in the art, for example by firstly converting to the acid chloride followed by reaction with an amine.

Certain of the substances of the invention can be made via cross-coupling techniques such as by reaction of a compound containing a bromo-substituent attached to e.g. an aromatic ring, with e.g. a boronic acid derivative, an olefin or a tin derivative by methods well-known in the art, for example by the methods described in certain of the Preparations below.

Certain of the substances of the invention having an electrophilic substituent can be made via halogen/metal exchange followed be reaction with an electrophilic reagent. For example a bromo-substituent may react with a lithiating reagent such as n-butyllithium and subsequently an electrophilic reagent such as CO₂, an aldehyde or ketone, to give respectively an acid or an alcohol.

Substances of the invention are available by either the methods described herein in the Methods and Examples or suitable adaptation thereof using methods known in the art. It is to be understood that the synthetic transformation methods mentioned herein may be carried out in various different sequences in order that the desired compounds can be efficiently assembled. The skilled chemist will exercise his judgement and skill as to the most efficient sequence of reactions for synthesis of a given target compound.

### Examples and Preparations

Melting points were determined using a Gallenkamp melting point apparatus and are uncorrected. Nuclear magnetic resonance data were obtained using a Varian Unity 300 or Varian Inova 400 spectrometer, and are quoted in parts per million from tetramethylsilane. Mass spectral data were obtained on a Finnigan Mat. TSQ 7000 or a Fisons Instruments Trio 1000. The calculated and observed ions quoted refer to the isotopic composition of lowest mass. Reference to “ether” in this section should be read as diethyl ether, unless specified otherwise. “Ph” represents the phenyl group. “Bn” represents the benzyl group. “Me” represents the methyl group. “TLC” means thin layer chromatography. “RT” means room temperature. “EtOAc” means ethyl acetate. Other abbreviations are standard and well-known in the art. Nomenclature has been allocated using the IUPAC NamePro software available from Advanced Chemical Development Inc.

### Example 1

### N″-(5-Methyl-2-pyridinyl)guanidine (I; R¹═CH₃; R²═R³═H)

Trifluoroacetic acid (2 ml) was added with care to tert-butyl N-[(tert-butoxycarbonyl)amino][(5-methyl-2-pyridinyl)imino]methylcarbamate (111 mg, 0.32 mmol) and the solution stirred at RT for 2 h, diluted with toluene and evaporated to dryness. The solid was azeotroped with methylene chloride, and recrystallised from methanol to give the trifluoroacetic acid salt of N″-(5-methyl-2-pyridinyl)guanidine as a cream-coloured solid (32 mg, 0.1 mmol):

¹H (δ, d₆-DMSO, 300 MHz); 2.2 (3H, s), 6.95 (1H, d), 7.7 (1H, d), 8.1 (1H, s), 8.35 (4H, br s), 11.05 (1H, br s); LRMS 151 (MH).

Other compounds of formula (I; R4 is N═C(NH2)2) prepared by the same method are listed in Table 1 below.

TABLE 1
NB all as trifluoroacetic acid salts unless noted otherwise

| Ex- | | | | | | | |
|---|---|---|---|---|---|---|---|
| am- | | | | | | | |
| ple | R³ | R² | R¹ | Mp ° C. | Elemental Analysis | LRMS | ¹H, δ |
| 2 | H | H | Cl | — | Found: C, 32.46; H, 2.87; N, | — | (DMSO-d₆, 300MHz) 7.1(1H, d), |
| | | | | | 18.08. Calcd for | | 8.0(1H, dd), 8.1-8.4(5H, br m) |
| | | | | | C₆H₇ClN₄.CF₃CO₂H + 0.25 | | |
| | | | | | CH₂Cl₂: C, 32.40; H, 2.80; N, | | |
| | | | | | 18.32 | | |
| 3 | H | H | Br | — | — | 215, 217 | (DMSO-d₆, 300MHz) 7.0(1H, dd), |
| | | | | | | (MH) | 8.05(1H, dd), 8.3(4H, br s), 8.4(1H, |
| | | | | | | | d), 11.4(1H, br s) |
| 4 | H | Ph | H | 156-8 | Found: C, 51.39; H, 3.96; N, | 213 (MH) | (DMSO-d₆, 300MHz) 7.25(1H, s), |
| | | | | | 17.06. Calcd for | | 7.45-7.6(4H, m), 7.75(2H, d), |
| | | | | | C₁₂H₁₂N₄.CF₃CO₂H: C, | | 8.25(2H, br s), 8.35(2H, d), 11.4(1H, |
| | | | | | 51.53; H, 4.02; N, 17.17 | | br s) |
| 5 | H | CONHCH₂Ph | H | — | — | 270 (MH), | (DMSO-d₆, 300MHz) |
| | | | | | | 539 | 4.5(2H, d), 7.2-7.35(4H, m), |
| | | | | | | (M₂H) | 7.4(1H, s), 7.6(1H, d), 8.3(4H, |
| | | | | | | | br s), 8.4(1H, d), 9.4(1H, dd), |
| | | | | | | | 11.1(1H, br s) |
| 6 | Cl | H | Cl | — | — | 205, 207 | (DMSO-d₆, 300MHz) |
| | | | | | | (MH) | 8.35(1H, d), 8.5(5H, br s), |
| | | | | | | | 9.9(1H, br s) |
| 7^{(a)} | Br | H | Cl | — | Found: C, 24.84; H, 2.39; N, | 249, 251, | (DMSO-d₆, 300MHz) |
| | | | | | 18.67. Calcd for | 253 (MH) | 8.4(1H, s), 8.2-8.8(5H, br s), |
| | | | | | C₆H₆BrClN₄.HCl + 0.1 | | 9.8(1H, s) |
| | | | | | CH₂Cl₂: C, 24.88; H, 2.46; N, | | |
| | | | | | 19.03 | | |
| 8 | Cl | H | Br | — | — | 249, 251, | (CF₃CO₂D, 300MHz) 8.05(1H, |
| | | | | | | 253 (MH) | s), 8.35(1H, s), 11.45(5H, s) |
| 9 | E-CH═CHCO₂H | H | Cl | 207-9 | Found: C, 37.2; H, 2.86; N, | 241, 243 | (CF₃CO₂D, 300MHz) 6.65(1H, |
| | | | | | 15.32. Calcd for | (MH) | d), 8.0(1H, d), 8.05(1H, s), |
| | | | | | C₉H₉ClN₄O₂.CF₃CO₂H + | | 8.35(1H, s), 11.45(6H, br s) |
| | | | | | 0.05 H₂O. C, 37.54; H, 2.95; | | |
| | | | | | N, 15.63 | | |
| 10 | CH₂CH₂CO₂H | H | Cl | 154-6 | Found: C, 37.15; H, 3.37; N, | 243, 245 | (CF₃CO₂D, 300MHz) 1.5-3.3 |
| | | | | | 15.56. Calcd for | (MH) | (2H, m), 3.3-3.4(2H, m), |
| | | | | | C₉H₁₁ClN₄O₂.CF₃CO₂H: C, | | 8.15(1H, s), 8.55(1H, s) |
| | | | | | 37.04; H, 3.39; N, 15.71 | | |
| 11 | E-CH═CHCONHMe | H | Cl | 208-210 | — | 254, 256 | (DMSO-d₆, 300MHz) 2.7(3H, |
| | | | | | | (MH); | d), 6.7(1H, d), 7.5(1H, d), 8.0- |
| | | | | | | 507, 509 | 8.3(6H, m), 8.4(1H, d), 9.8(1H, |
| | | | | | | (M₂H) | br s) |
| 12 | E- | H | Cl | — | Found: C, 48.53; H, 3.88; N, | 330, 332 | (DMSO-d₆, 300MHz) 4.4(2H, |
| | CH═CHCONHCH₂Ph | | | | 15.29. Calcd for | (MH) | d), 6.8(1H, d), 7.2-7.35(5H, m), |
| | | | | | C₁₆H₁₆ClN₅O.CF₃CO₂H + | | 7.6(1H, d), 8.2(1H, d), 8.2- |
| | | | | | 0.05 H₂O. C, 48.85; H, 3.94; | | 8.35(4H, br s), 8.4(1H, d), 8.7(1H, t), |
| | | | | | N, 15.65 | | 9.95(1H, s) |
| 13 | E-CH═CHCO-(3- | H | Cl | — | Found: C, 42.79; H, 4.35; N, | 324, 326 | (DMSO-d₆ + 1 drop CF₃CO₂D, |
| | hydroxypiperidino) | | | | 14.64. Calcd for | (MH) | 400MHz) 1.1-1.55(2H, m), 1.6- |
| | | | | | C₁₄H₂₈ClN₅O₂ + 1.25 | | 1.95(2H, m), 2.75(0.5H, dd), |
| | | | | | CF₃CO₂H: C, 42.50; H, 4.16; | | 3.1-3.2(0.5H, m), 3.3-3.45(1.5H, |
| | | | | | N, 15.02 | | m), 3.5-3.65(1H, m), 3.8(0.5H, |
| | | | | | | | dd), 3.95(0.5H, d), 4.4(0.5H, dd), |
| | | | | | | | 7.45(1H, dd), 7.6(1H, d), 8.2 |
| | | | | | | | (1H, br s), 8.35(1H, d), 8.5(1H, |
| | | | | | | | d) |
| 14 | E- | H | Cl | 148-150 | Found: 49.27; H, 4.10; N, | 344, 346 | (DMSO-d₆, 300MHz) 2.9 & |
| | CH═ | | | | 15.00. Calcd for | (MH) | 3.15(3H, both s), 4.6 & 4.85(2H, |
| | CHCON(Me)CH₂Ph | | | | C₁₇H₃₅ClN₅O.CF₃CO₂H + | | both s), 7.1-7.4(5H, m), 7.5(1H, app. |
| | | | | | 0.25 H₂O: C, 49.35; H, 4.25; | | dd), 7.65(1H, app. dd), 8.0- |
| | | | | | N, 15.14 | | 8.2(3H, m), 8.35(1H, app. dd), 8.55 |
| | | | | | | | (1H, app. dd), 9.9(1H, br s) |
| 15 | E- | H | Cl | — | Found: C, 41.40; H, 4.03; N, | 310, 312 | (DMSO-d₆, 300MHz) 3.55- |
| | CH═CHCO(morpho- | | | | 15.36. Calcd for | (MH) | 3.8(8H, br m), 7.45(1H, d), 7.6(1H, |
| | lino) | | | | C₁₃H₁₆ClN₅O₂ + 1.5 | | d), 8.1-8.25(4H, br s), 8.4(1H, |
| | | | | | CF₃CO₂H: C, 41.15; H, 3.84; | | d), 8.55(1H, d), 9.95(1H, br s) |
| | | | | | N, 15.48 | | |
| 16 | E-CH═CHSO₂NHMe | H | Cl | — | — | 290, 292 | (DMSO-d₆, 300MHz) 2.55(3H, |
| | | | | | | (MH) | d), 7.3(1H, q), 7.4(2H, s), 8.0- |
| | | | | | | | 8.2(4H, br m), 8.45(1H, d), 8.5(1H, |
| | | | | | | | d), 10.0-10.15(1H, m), |
| 17 | E-CH═CHPh | H | Cl | >275 | Found: C, 49.55; H, 3.62; N, | 273 (MH) | (DMSO-d₆, 300MHz) 7.2- |
| | | | | | 14.27. Calcd for | | 7.5(5H, m), 7.65(2H, d), 8.1-8.35 |
| | | | | | C₁₄H₁₃ClN₄.CF₃CO₂H: C, | | (5H, m), 8.35(1H, s), 10.0(1H, s) |
| | | | | | 49.38; H, 3.65; N, 14.49 | | |
| 18 | E-CH═CH(4- | H | Cl | >275 | Found: C, 49.04; H, 3.81; N, | 303, 305 | (DMSO-d₆, 300MHz) 3.8(3H, s), |
| | MeOC₆H₄) | | | | 13.09. Calcd for | (MH) | 7.0(2H, d), 7.05(1H, d), 7.4(1H, |
| | | | | | C₁₅H₁₅ClN₄O.CF₃CO₂H: C, | | d), 7.6(2H, d), 8.1-8.3(5H, m), |
| | | | | | 48.99; H, 3.87; N, 13.44 | | 9.9(1H, br s) |
| 19^{(b)} | E-CH═CH₂(2- | H | Cl | — | Found: C, 41.87; H, 3.05; N, | 274, 276 | (DMSO-d₆, 300MHz) 7.3- |
| | pyridyl) | | | | 14.75. Calcd for | (MH); | 7.4(1H, m), 7.4-7.5(2H, m), 7.75 |
| | | | | | C₁₃H₁₂ClN₅ + 1.75 | 547, 549 | (1H, d), 7.8-7.9(1H, m), 8.1- |
| | | | | | CF₃CO₂H: C, 41.87; | (M₂H) | 8.3(4H, br m), 8.35(1H, d), 8.45 |
| | | | | | H, 2.93; N, 14.80 | | (1H, d), 8.6-8.65(1H, m), |
| | | | | | | | 10.0(1H, br s) |
| 20 | E-CH═CH- | H | Cl | 156-158 | — | 279, 281 | (DMSO-d₆, 400MHz) 1.1- |
| | cyclohexyl | | | | | (MH) | 1.35(5H, m), 1.6-1.65(1H, m), 1.65- |
| | | | | | | | 1.8(4H, m), 2.1-2.2(1H, m) |
| | | | | | | | 6.45(2H, s), 8.1(1H, s), 8.15-8.25 |
| | | | | | | | (3H, br s), 8.25(1H, s), 9.0(1H, |
| | | | | | | | br s), 9.7(1H, br s) |
| 21 | E-CH═CH-(3,4- | H | Cl | — | Found: C, 45.96; H, 3.17; N, | 317 (MH) | (DMSO-d₆, 400MHz) 6.05(2H, |
| | methylenedioxyphenyl) | | | | 12.65. Calcd for | | s), 6.95(1H, d), 7.05-7.15(2H, |
| | | | | | C₁₅H₁₃ClN₄O₂ + 1.2 | | m), 7.3(1H, s), 7.4(1H, d), 8.1- |
| | | | | | CF₃CO₂H: C, 46.08; H, 3.16; | | 8.3(6H, m), 9.85(1H, br s) |
| | | | | | N, 12.35 | | |
| 22 | E-CH═CH(3-CN- | H | Cl | — | Found: C, 49.00; H, 3.35; N, | 298, 300 | (CF₃CO₂D, 400MHz) 7.1(1H, d), |
| | C₆H₄) | | | | 16.58. Calcd for | (MH) | 7.2(1H, d), 7.45-7.55(1H, m), |
| | | | | | C₁₅H₁₂ClN₅.CF₃CO₂H + 0.25 | | 7.6(1H, d), 7.75-7.8(2H, m), |
| | | | | | H₂O: C, 49.05; H, 3.27; N, | | 8.2(1H, s), 8.3(1H, s), 11.4(5H, s) |
| | | | | | 16.82 | | |
| 23 | C═CPh | H | Cl | 179-181 | Found: C, 49.76; H, 3.21; N, | 271, 273 | (DMSO-d₆, 400MHz) 7.45- |
| | | | | | 14.25. Calcd for | (MH) | 7.5(3H, m), 7.55-7.7(2H, m), |
| | | | | | C₁₄H₁₁ClN₄.CF₃CO₂H: C, | | 8.3(1H, d), 8.4(1H, d), 8.3-8.6(4H, |
| | | | | | 49.94; H, 3.14; N, 14.56 | | br s), 9.8(1H, br s) |
| 24 | OPh | H | Cl | 170-172 | Found: C, 44.61; H, 3.15; N, | 263, 265 | (DMSO-d₆, 400MHz) 7.2(2H, |
| | | | | | 14.58. Calcd for | (MH) | d), 7.3(2H, d), 7.5-7.6(2H, m), |
| | | | | | C₁₂H₁₁ClN₄O.CF₃CO₂H: C, | | 8.1(1H, s), 8.2-8.5(4H, br s), |
| | | | | | 44.63; H, 3.19; N, 14.87 | | 10.1(1H, br s) |
| 25 | OCH₂Ph | H | Cl | 176-8 | Found: C, 45.73; H, | 277, 279 | (DMSO-d₆, 400MHz) 5.35(2H, s), |
| | | | | | 3.57; N, 13.68. Calcd for | (MH) | 7.3-7.45(3H, m), 7.45-7.5(2H, m), |
| | | | | | C₁₃H₁₃ClN₄O.CF₃CO₂H + | | 7.8(1H, s), 7.9(1H, s), 8.0-8.7(4H, |
| | | | | | 0.5 H₂O + 0.05 | | br s), 9.7(1H, s) |
| | | | | | EtOAc: C, 45.76; H, | | |
| | | | | | 3.79; N, 14.04 | | |
| 26 | OCH₂CH₂OH | H | Cl | 169-171 | Found: C, 34.63; H, | 231, 233 | (DMSO-d₆, 400MHz) 3.75(2H, s), |
| | | | | | 3.46; N, 15.76. Calcd for | (MH) | 4.2(2H, s), 4.95(1H, s), 7.75(1H, s), |
| | | | | | C₈H₁₁ClN₄O₂.CF₃CO₂H + | | 7.9(1H, s), 8.0-8.6(4H, br s), |
| | | | | | 0.25 H₂O: C, 34.39; H, | | 9.7(1H, s) |
| | | | | | 3.61; N, 16.05 | | |
| 27 | OCH₂CH₂OMe | H | Cl | 120-122 | Found: C, 36.75; H, | 245, 247 | (DMSO-d₆, 300MHz) 3.25(3H, s — |
| | | | | | 3.87; N, 15.18. Calcd for | (MH) | under water peak by CF₃CO₂D |
| | | | | | C₉H₃ClN₄O₂.CF₃CO₂H + 0.2 | | exchange), 3.7(2H, t), 4.35(2H, t), |
| | | | | | H₂O: C, 36.47; H, | | 7.75(1H, d), 7.9(1H, d), 8.1-8.7(4H, |
| | | | | | 4.01; N, 15.46 | | br s), 9.7(1H, s) |
| 28 | OCH₂CONCH₂Ph | H | Cl | 209-211 | Found: C, 45.23; H, | 334, 336 | (CF₃CO₂D, 400MHz) 4.35(2H, s), |
| | | | | | 3.80; N, 15.23. Calcd for | (MH) | 4.9(2H, s), 7.15-7.2(2H, m), |
| | | | | | C₁₅H₁₆ClN₅O₂.CF₃CO₂H: | | 7.2-7.3(3H,m), 7.35(1H, s), 7.95(1H, |
| | | | | | C, 45.60; H, 3.83; N, | | s) |
| | | | | | 15.64 | | |
| 29 | OCH₂(3-CO₂Me- | H | Cl | 187-188.5 | Found: C, 45.26; H, | 335, 337 | (CF₃CO₂D, 400MHz) 4.0(3H, s), |
| | C₆H₄) | | | | 3.54; N, 12.29: Calcd for | (MH) | 5.2(2H, s), 7.4(1H, s), 7.5(1H, t), |
| | | | | | C₁₅H₁₅ClN₄O₃.CF₃CO₂H: | | 7.6(1H, d), 7.85(1H, s), 8.1-8.05(2H, |
| | | | | | C, 45.50; H, 3.59; N, | | m) |
| | | | | | 12.48 | | |
| 30 | CH₂OPh | H | Cl | 177-180 | Found: C, 44.60; H, | 277, 279 | (DMSO-d₆, 300MHz) 5.15(2H, s), |
| | | | | | 3.60; N, 14.03. Calcd for | (MH) | 7.0(1H, t), 7.05(2H, d), 7.3(2H, dd), |
| | | | | | C₁₃H₁₃ClN₄O.CF₃CO₂H + | | 8.1(1H, d), 8.2-8.4(5H, m), |
| | | | | | 0.5 H₂O: C, 45.07; H, | | 9.6(1H, br s) |
| | | | | | 3.78; N, 14.02 | | |
| 31 | Cl | Cl | Cl | 210.5-212.5 | Found: C, 27.36; H, | 239, 241, | (DMSO-d₆, 300MHz) 8.3(4H, br s), |
| | | | | | 1.71; N, 15.45. Calcd for | 243, 245 | 8.5(1H, s), 10.0(1H, br s) |
| | | | | | C₈H₆Cl₃F₃N₄O₂ + 1.05 | | |
| | | | | | CF₃CO₂H: C, 27.08; H, | (MH) | |
| | | | | | 1.70; N, 15.60° | | |
| 32 | Cl | Me | Cl | 201-3 | Found: C, 32.10; H, | 219, 221, | (DMSO-d₆, 300MHz) 8.3(1H, s), |
| | | | | | 2.75; N, 16.74. Calcd for | 223 (MH) | 8.5(3H, br s), 9.85(1H, br s) |
| | | | | | C₇H₈Cl₂N₄.CF₃CO₂H: C, | | |
| | | | | | 32.45; H, 2.72; N, 16.82 | | |
| 33 | Cl | CH₂OPh | Cl | 164-166 | Found: C, 42.10, H, | 311, 313 | (DMSO-d₆, 300MHz) 5.25(2H, s), |
| | | | | | 3.04; N, 13.03. Calcd for | (MH) | 6.95-7.05 (3H, m), 7.25-7.35(2H, |
| | | | | | C₁₃H₁₂Cl₂N₄O.CF₃CO₂H: | | m), 8.3-8.5(5H, br m), 10.0(1H, br s) |
| | | | | | C, 42.38; H, 3.08; N, | | |
| | | | | | 13.18 | | |
| 34 | Cl | CH₂NMeBn | Cl | 202-5 | Found: C, 40.07; H, | 338, 339, | (DMSO-d₆ + 1 drop of CF₃CO₂D, |
| | | | | | 3.27; N, 12.19. Calcd for | 341 (MH) | 300MHz) 2.8(3H, s), 4.4-4.55(4H, |
| | | | | | C₁₅H₁₇Cl₂N₅.2CF₃CO₂H: | | m), 7.4-7.5(3H, m), 7.55-7.6(2H, |
| | | | | | C, 40.29; H, 3.38; N, | | m), 8.4(1H, s) |
| | | | | | 12.37 | | |
| 35 | Cl | CH₂N(CH₂)₄ | Cl | 161-3 | Found: C, 34.67; H, | 288, 290, | (CF₃CO₂D, 400MHz) 2.0-2.1(2H, |
| | | | | | 3.28; N, 13.33. Calcd for | 292 (MH) | m), 2.1-2.3(2H, m), 3.25-3.35(2H, |
| | | | | | C₁₁H₁₅Cl₂N₅.2CF₃CO₂H: | | m), 3.65-3.75(2H, m), 4.65(2H, s), |
| | | | | | C, 34.90; H, 3.32; N, | | 8.3(1H, s) |
| | | | | | 13.57 | | |

^{(a)}HCl salt
^{(b)}appeared to be mixture of mono- and bis-triflate salt
^{(c)}bis-TFA salt

### Example 36

### 3-((E)-2-{5-Chloro-2-[(diaminomethylene)amino]-3-pyridinyl}ethenyl)benzoic acid (I: R¹═Cl; R²═H; R³═E-CH═CH(3-C₆H₄—CO₂H)

N″-5-chloro-3-[(E)-2-(3-cyanophenyl)ethenyl]-2-pyridinylguanidine (85 mg, 0.2 mmol) was heated to relux in conc. HCl (1.5 ml) and acetic acid (0.5 ml) for 48 h. Solvent was removed in vacuo and the residue azeotropically dried with toluene to give a light brown solid which was triturated with diethyl ether to give 3-((E)-2-{5-chloro-2-[(diaminomethylene)amino]-3-pyridinyl}ethenyl)benzoic acid as an off-white solid (65 mg, 0.2 mmol):

¹H (δ, CF₃CO₂D, 400 MHz) 7.2 (1H, d), 7.4 (1H, d), 7.5 (1H, t), 7.8 (1H, d), 8.1 (1H, d), 8.3 (1H, s), 8.45 (1H, s), 8.55 (1H, s);

LRMS 317, 319 (MH);

M. Pt.>275° C.;

El. Anal.—Found: C, 49.36; H, 4.24; N, 15.51. Calcd for C₁₅H₁₃ClN₄O₂.HCl+⅔ water: C, 49.35; H, 4.23; N, 15.35.

### Preparation 1: t-Butyl (E)-3-(2-amino-5-chloro-3-pyridinyl)-2-propenoate

A mixture of 3-bromo-5-chloro-2-pyridinamine (C. W. Murtiashaw, R. Breitenbach, S. W. Goldstein, S. L. Pezzullo, J. Quallich, R. Sarges, J. Org. Chem., 1992, 57, 1930) (8.56 g, 41.4 mmol), t-butyl acrylate (12 ml, 82 mmol), tri-o-tolylphosphine (2.92 g, 9.6 mmol) and palladium acetate (540 mg, 2.4 mmol) in triethylamine (130 ml) was heated in a sealed bomb to 150° C. for 10 hours. The reaction mixture was filtered, the residue washed with EtOAc and the combined filtrates evaporated to a dark brown oil. Purification by column chromatography upon silica gel using hexane-EtOAc (7:3) as eluant and subsequent crystallisation from hexane at −78° C. gave the title compound as a bright yellow solid (4.75 g, 18.6 mmol).

¹H (δ, CDCl₃, 300 MHz) 1.5 (9H, s), 4.7 (2H, br s), 6.3 (1H, d), 7.45 (1H, d), 7.55 (1H, s), 8.0 (1H, s);

LRMS 255, 257 (MH);

El. Anal.—Found: C, 56.55; H, 5.94; N, 10.91. Calcd for C₁₂H₁₅ClN₂O₂: C, 56.58; H, 5.94; N, 10.99.

### Preparation 2: (E)-3-(2-Amino-5-chloro-3-pyridinyl)-2-propenoic acid

t-Butyl (E)-3-(2-amino-5-chloro-3-pyridinyl)-2-propenoate (2 g, 7.8 mmol), was stirred in 3 ml temperature for 1 hour. The reaction mixture was diluted with toluene, evaporated to dryness, and the residue triturated with diethyl ether to yield the title compound as a pale yellow solid (1.89 g, 6.0 mmol).

¹H (δ, d₆-DMSO, 300 MHz) 5.0-7.5 (br s), 6.5 (1H, d), 7.65 (1H, d), 7.95 (1H ,s), 8.0 (1H, s);

LRMS 199, 201 (MH);

El. Anal.—Found: C, 38.41; H, 2.49; N, 8.87. Calcd for C₈H₇ClN₂O₂.CF₃CO₂H: C, 38.42; H, 2.58; N, 8.96.

### Preparation 3: t-Butyl 3-(2-amino-5-chloro-3-pyridinyl)propanoate

To a solution of t-butyl (E)-3-(2-amino-5-chloro-3-pyridinyl)-2-propenoate (500 mg, 2.0 mmol) in ethanol (10 ml) at RT was added sodium borohydride (317 mg, 8.4 mmol) portionwise and the mixture stirred for 16 h. After the addition of water, the ethanol removed in vacuo and the mixture extracted with diethyl ether. The ethereal extracts were dried over MgSO₄, evaporated to dryness and purified by column chromatography upon silica gel using hexane-EtOAc (7:3) as eluant to give t-butyl 3-(2-amino-5-chloro-3-pyridinyl)propanoate as a colourless oil (340 mg, 1.3 mmol).

¹H (δ, CDCl₃, 300 MHz) 1.4 (9H, s), 2.5 (2H, t), 2.7 (2H, t), 4.6 (2H, br s), 7.2 (1H, d), 7.9 (1H, d);

LRMS 257, 259 (MH).

### Preparation 4: (E)-3-(2-Amino-5-chloro-3-pyridinyl)-N-methyl-2-propenamide

1-Hydroxybenzotriazole.H₂O (196 mg, 1.4 mmol), methylamine.HCl (114 mg, 1.7 mmol), Hunig's base (1.58 ml, 9.1 mmol), 1-(3-dimethylaminopropyl)-3-ethylcarbodiimide.HCl (555 mg, 2.8 mmol) and (E)-3-(2-amino-5-chloro-3-pyridinyl)-2-propenoic acid.CF₃CO₂H (438 mg, 1.4 mmol) were combined in DMF (5 ml) and stirred at RT for 16 h. The reaction mixture was poured into water (50 ml), extracted with EtOAc (3×20 ml), and the combined organic extracts washed with saturated brine, dried over MgSO₄, and concentrated to a yellow solid. Trituration with diethyl ether gave the title compound (198 mg, 0.9 mmol).

¹H (δ, d₆-DMSO, 300 MHz) 2.7 (3H, d), 6.25 (2H, br s), 6.45 (1H, d), 7.4 (1H, d), 7.6(1H, s), 7.87 (1H, br s), 7.9 (1H, s);

LRMS 212, 214 (MH);

M. Pt. 188-190° C.;

El. Anal.—Found: 50.88; H, 4.81; N, 19.75. Calcd for C₉H₁₀ClN₃O: C, 51.07; H, 4.76; N, 19.86.

The following compounds of Preparations 5-9 were prepared similarly:

### Preparation 5: 2-amino-N-benzylisonicotinamide

The title compound was prepared from 2-aminoisonicotinic acid (L. W. Deady, O. L. Korytsky, J. E. Rowe, Aust. J. Chem., 1982, 35, 2025) and benzylamine:

¹H (δ, d,-DMSO, 300 MHz) 4.4 (2H, d), 6.05 (2H, s), 6.8 (1H, s), 6.8 (1H, d), 7.2-7.4 (5H, m), 8.0 (1H, d), 9.0 (1H, br t);

LRMS 228 (MH); 455 (M₂H).

### Preparation 6: (E)-3-(2-Amino-5-chloro-3-pyridinyl)-N-benzyl-2-propenamide

The title compound was prepared from (E)-3-(2-amino-5-chloro-3-pyridinyl)-2-propenoic acid and benzylamine as a yellow solid:

¹H (δ, d₆-DMSO, 300 MHz) 4.2 (2H, d), 6.25 (2H, br s), 6.6 (1H, d), 7.2-7.35 (5H, m), 7.45 (1H, d), 7.65 (1H, s), 7.95 (1H, s), 8.4 (1H, br t);

LRMS 288, 290 (MH); 575, 577, 579 (M₂H);

El. Anal.—Found: C, 62.32; H, 4.93; N, 14.59. Calcd for C₁₅H₁₄ClN₃O: C, 62.61; H, 4.90; N, 14.60.

### Preparation 7: (E)-3-(2-Amino-5-chloro-3-pyridinyl)-1-(3-hydroxypiperidino)-2-propen-1-one

The title compound was prepared from (E)-3-(2-amino-5-chloro-3-pyridinyl)-2-propenoic acid and 3-hydroxypiperidine as a white solid:

¹H (δ, d₆-DMSO, 400 MHz) 1.25-1.55 (2H, m), 1.6-1.95 (2H, m), 2.6-3.15 (1H, m), 3.2-4.3 (4H, m), 4.8-4.85 (1H, m), 6.3 (2H, s), 7.1-7.2 (1H, m), 7.5 (1H, d), 7.9 (1H, s), 8.0 (1H, d);

LRMS 282, 284 (MH); 563, 565, 567 (M₂H).

### Preparation 8: (E)-3-(2-Amino-5-chloro-3-pyridinyl)-N-benzyl-N-methyl-2-propenamide

The title compound was prepared from (E)-3-(2-amino-5-chloro-3-pyridinyl)-2-propenoic acid and N-methyl benzylamine as a yellow solid following crystallisation from diisopropyl ether:

¹H (δ, CDCl₃, 300 MHz) 3.1 (3H, s), 4.6-4.85 (4H, m), 6.8-6.85 (1H, m), 7.2-7.45 (6H, m), 7.5-7.7 (1H, m), 7.95-8.05 (1H, m);

LRMS 302, 304 (MH); 603 (M₂H);

M. Pt. 106-109° C.;

El. Anal.—Found: C, 63.33; H, 5.29; N, 13.67. Calcd for C₁₆H₁₆ClN₃O: C, 63.68; H, 5.34; N, 13.93.

### Preparation 9: (E)-3-(2-Amino-5-chloro-3-pyridinyl)-1-morpholino-2-propen-1-one

The title compound was prepared from (E)-3-(2-amino-5-chloro-3-pyridinyl)-2-propenoic acid and morpholine as a yellow solid following crystallisation from isopropyl alcohol and trituration with diisopropyl ether:

¹H (δ, CDCl₃, 400 MHz) 3.6-3.8 (8H, m), 4.8 (2H, br s), 6.8 (1H, d), 7.55 (1H, s), 7.6 (1H, d), 8.0 (1H, s);

LRMS 268, 270 (MH).

### Preparation 10: (E)-2-(2-Amino-5-chloro-3-pyridinyl)-N-methyl-1-ethenesulphonamide

A mixture of 3-bromo-5-chloro-2-pyridinamine (414 mg, 2 mmol), N-methyl ethene sulphonamide (266 mg, 2.2 mmol) and triethylamine (555 μl, 4 mmol), palladium acetate (18 mg, 0.08 mmol) and tri-o-tolylphosphine (50 mg, 0.16 mmol) in DMF (0.5 ml) in a Teflon™ pressure vessel was microwaved for 30 sec (full power), allowed to cool to RT and irradiated for a further 30 sec. After allowing to cool, the reaction mixture was diluted with water, extracted with EtOAc and the organic phase washed with saturated brine, dried over MgSO₄, and concentrated to a brown semi-solid. Purification by column chromatography on silica gel eluting with methylene chloride-methanol (95:5), and then crystallisation from methanol gave the title compound (130 mg, 0.5 mmol):

¹H (δ, d₆-DMSO, 400 MHz) 2.5 (3H, s), 6.5 (2H, br s), 6.95 (1H, br s), 7.1 (1H, d), 7.35 (1H, d), 7.95 (1H, s), 8.0 (1H, s);

LRMS 248, 250 (MH);

M. Pt. 194-8° C.;

El. Anal.—Found: C, 38.61; H, 4.04; N, 16.61. Calcd for C₈H₁₀ClN_{O}₂S: C, 38.79; H, 4.07; N, 16.97.

The following compounds of Preparations 11-15 were prepared similarly:

### Preparation 11: 5-Chloro-3-[(E)-2-phenylethenyl]-2-pyridinamine

The title compound was prepared from 3-bromo-5-chloro-2-pyridinamine and stryene. Purification by column chromatography on silica gel eluting with hexane-EtOAc (70:30) gave an oil which crystallised from hexane to give 5-chloro-3-[(E)-2-phenylethenyl]-2-pyridinamine as a yellow solid:

¹H (δ, CDCl₃, 300 MHz) 4.5 (2H, br s), 6.9(1H, d), 7.0 (1H, d), 7.2-7.55 (5H, m), 7.6 (1H, s), 8.0 (1H, s);

LRMS 231, 233 (MH);

El. Anal.—Found C, 67.33; H, 4.78; N, 12.00. Calcd for C₁₃H₁₁ClN₂: C, 67.68; H, 4.81; N, 12.14.

### Preparation 12: 5-Chloro-3-[(E)-2-(4-methoxyphenyl)ethenyl]-2-pyridinamine

The title compound was prepared from 3-bromo-5-chloro-2-pyridinamine and 4-methoxystryene. Purification by column chromatography on silica gel eluting with hexane-EtOAc (80:20) gave an oil which crystallised from hexane to give a yellow solid:

¹H (δ, CDCl₃, 300 MHz) 3.8 (3H, s), 4.5 (2H, br s), 6.75 (1H, d), 6.85-7.0 (3H, m), 7.4 (2H, d), 7.55 (1H, d), 7.95 (1H, d);

LRMS 261, 263 (MH).

### Preparation 13: 5-Chloro-3-C(E)-2-(2-pyridinyl)ethenyl]-2-pyridinamine

The title compound was prepared from 3-bromo-5-chloro-2-pyridinamine and 2-vinylpyridine. Purification by column chromatography on silica gel eluting with methylene chloride-methanol (95:5) and repeated using hexane-EtOAc (70:30 to 50:50) as eluant gave a yellow solid:

¹H (δ, CDCl₃, 300 MHz) 4.7 (2H, br s), 7.05 (1H, d), 7.2-7.35 (2H, m), 7.55 (1H, d), 7.6-7.7 (2H, m), 8.0 (1H, d), 8.6 (1H, d);

LRMS 232, 234 (MH).

### Preparation 14: 5-Chloro-3-[(E)-2-cyclohexylethenyl]-2-pyridinamine

The title compound was prepared from 3-bromo-5-chloro-2-pyridinamine and vinylcyclohexane. Purification by column chromatography on silica gel eluting with hexane-EtOAc (80:20) gave a pale yellow oil. An analytical sample was prepared by crystallisation from hexane:

¹H (δ, CDCl₃, 300 MHz) 1.1-1.4 (5H, m), 1.5-1.8 (5H, m), 2.1-2.2 (1H, m), 4.5 (2H, br s), 6.0-6.2 (2H, m), 7.4 (1H, d), 7.9 (1H, d);

LRMS 237, 239 (MH);

El. Anal.—Found: C, 65.85; H, 7.29; N, 11.84. Calcd for C₁₃H₁₇ClN₂: C, 65.95; H, 7.24; N, 11.83.

### Preparation 15: 3-[(E)-2-(2-Amino-5-chloro-3-pyridinyl)ethenyl]benzonitrile

The title compound was prepared from 3-bromo-5-chloro-2-pyridinamine and 3-cyanostyrene. Methylene chloride extracts of crude reaction mixture were concentrated and the desired product purified by column chromatography on silica gel eluting with methylene chloride-methanol (98:2) to give a yellow solid:

¹H (δ, CDCl₃, 300 MHz) 6.4 (2H, br s), 7.2 (1H, d), 7.35 (1H, d), 7.55 (1H, t), 7.7 (1H, d), 7.8-7.9 (3H, m), 8.15 (1H, s);

LRMS 256, 258 (MH);

M. Pt.>275° C.;

El. Anal.—Found: C, 65.49; H, 3.96; N, 16.21. Calcd for C₁₄H₁₀ClN₃: C, 65.76; H, 3.94; N, 16.43.

### Preparation 16: 3-[(E)-2-(1,3-Benzodioxol-5-yl)ethenyl]-5-chloro-2-pyridinamine

A solution of 3-bromo-5-chloro-2-pyridinamine (318 mg, 1.5 mmol), [(E)-2-(1,3-benzodioxol-5-yl)ethenyl](tributyl)stannane (250 mg, 1.7 mmol) (A. J. Bridges, A. Lee, C. E. Schwartz, M. J. Towle, B. A. Littlefield, Bioorg. Med. Chem., 1993, 1, 403), palladium acetate (19 mg, 0.08 mmol) and tri-o-tolylphosphine (50 mg, 0.16 mmol) in DMF (0.5 ml) and triethylamine (0.5 ml) in a teflon pressure vessel was heated in a microwave (full power) for 20 sec, allowed to cool to RT heated in a microwave for a further 20 sec and then 1 min 20 sec. After allowing to cool, the reaction mixture was poured into water (20 ml) and extracted with EtOAc (3×20 ml). The combined extracts were washed with water (2×20 ml), dried over MgSO₄ and concentrated. Recrystallisation from EtOAc-hexane gave the title compound as a brown solid (170 mg, 0.6 mmol):

¹H (δ, CDCl₃, 300 MHz) 4.55 (2H, br s), 6.0 (2H, s), 6.7 (1H, d), 6.8 (1H, d), 6.9-7.0 (2H, m), 7.05 (1H, s), 7.55 (1H, s), 7.95 (1H, s);

LRMS 275, 277 (MH).

### Preparation 17: 5-Chloro-3-(2-phenylethynyl)-2-pyridinamine

A solution of 3-bromo-5-chloro-2-pyridinamine (414 mg, 2.0 mmol), phenyl acetylene (225 mg, 2.2 mmol), copper (I) chloride (16 mg, 0.16 mmol), triethylamine (555 μl, 4.0 mmol) and dichlorobis(triphenylphosphine)palladium (II) (32 mg, 0.05 mmol) in DMF (0.5 ml) in a teflon pressure vessel was heated in a microwave (full power) for 30 sec, allowed to cool to RT and reheated for a further 30 sec. After cooling to RT, the reaction mixture was partioned between water-EtOAc, and the organic phase washed with sat. brine, dried over MgSO₄, and concentrated. Purification by column chromatography on silica gel eluting with methylene chloride-methanol (99:1) and subsequent crystallisation from hexane gave the title compound as a yellow solid (130 mg, 0.6 mmol):

¹H (δ, CDCl₃, 300 MHz) 5.0 (2H, br s), 7.3-7.4 (3H, m), 7.45-7.55(2H, m), 7.6 (1H, s), 8.0 (1H, br s);

LRMS 229, 231 (MH);*

M. Pt. 119-119° C.;

El. Anal.—Found: C, 66.53; H, 3.91; N, 12.00. Calcd for C₁₃H₉ClN₂+⅓ water: C, 66.70; H, 4.13; N, 11.97.

### Preparation 18: 5-Chloro-3-phenoxy-2-pyridinamine

3-Bromo-5-chloro-2-pyridinamine (520 mg, 2.5 mmol), phenol (2.0 g, 21.3 mmol), potassium hydroxide (flakes, 85%, 600 mg, 9.1 mmol) and anhydrous copper (II) sulphate (100 mg, 0.6 mmol) and dimethoxyethane (250 μl) were heated together at 140° C. for 2 h, allowed to cool and the mixture poured into water (50 ml). EtOAc extracts (5×15 ml) were filtered through celite and extracted into 2N HCl (4×10 ml). The combined aqueous extracts were basified with NaOH and re-extracted into EtOAc (3×20 ml), dried over MgSO₄, and concentrated to a brown oil (230 mg). Purification by column chromatography on silica gel eluting with hexane-EtOAc (80:20) gave the title compound as a white solid (136 mg, 0.6 mmol). An analytical sample was prepared by crystallisation from hexane:

¹H (δ, CDCl₃, 400 MHz) 4.7 (2H, br s), 6.95 (1H, s), 7.05 (2H, d), 7.2 (1H, t), 7.4 (2H, dd), 7.8 (1H, s);

LRMS 221, 223 (MH);

El. Anal.—Found: C, 59.87; H, 4.11; N, 12.64. Calcd for C₁₁H₉ClN₂O: C, 59.87; H, 4.11; N, 12.70.

### Preparation 19: 3-(Benzyloxy)-5-chloro-2-pyridinamine

(This compound is known and synthesis by a different route is disclosed—J. A. Bristol, I. Gross, R. G. Lovey, Synthesis, 1981, 971)

The title compound was prepared from 3-bromo-5-chloro-2-pyridinamine and benzyl alcohol using the conditions of preparation 18:

¹H (δ, CDCl₃, 300 MHz) 4.65 (2H, br s), 5.0 (2H, s), 6.95 (1H, s), 7.3-7.45 (5H, m), 7.6 (1H, s);

LRMS 235, 237 (MH);

El. Anal.—Found: C, 61.32; H, 4.70; N, 11.88. Calcd for C₁₂H₁₁ClN₂O: C, 61.41; H, 4.72; N, 11.94.

### Preparation 20: 2-[(2-Amino-5-chloro-3-pyridinyl)oxy]-1-ethanol

The title compound was by the method of G. Mattern (Helv. Chimica Acta, 1977, 60, 2062):

¹H (δ, CDCl₃, 300 MHz) 2.0(1H, br s), 3.95-4.05 (2H, m), 4.1-4.2 (2H, m), 4.7 (2H, br s), 6.95 (1H, s), 7.7 (1H, br s);

LRMS 189, 191 (MH).

### Preparation 21: 5-Chloro-3-(2-methoxyethoxy)-2-pyridinamine

The title compound was by the method of G. Mattern (Helv. Chimica Acta, 1977, 60, 2062):

¹H (δ, CDCl₃, 300 MHz) 3.4 (3H, s), 3.7-3.8 (2H, m), 4.1-4.2 (2H, m), 4.7 (2H, br s), 6.95 (1H, s), 7.65 (1H, s);

LRMS 203, 205 (MH).

### Preparation 22: 2-[(2-Amino-5-chloro-3-pyridinyl)oxy]-N-benzylacetamide

The title compound was prepared by the method of P. Nedenskov, N. Clauson-Kaas, J. Lei, H.-N. Heide, G. Olsen and G. Jansen (Acta Chemica Scandinavica, 1969, 23, 1791) from 2-amino-5-chloro-3-pyridinol (G. Mattern, Helv. Chimica Acta, 1977, 60, 2062) and N-benzyl-α-chloroacetamide. Sand coloured solid:

¹H (δ, CDCl₃, 400 MHz) 4.5-4.55 (2H, m), 4.6 (2H, s), 4.65 (2H, br s), 6.7 (1H, br s), 6.9 (1H, s), 7.2-7.35 (5H, m), 7.7 (1H, s);

LRMS 292, 294 (MH);

El. Anal.—Found: C, 56.92; H, 4.75; N, 13.93. Calcd for C₁₄H₁₄ClN₃O₂+0.25 water: C, 56.76; H, 4.93; N, 14.18.

### Preparation 23: Methyl 3-[(2-amino-5-chloro-3-pyridinyl)oxy]methylbenzoate

The title compound was prepared using the method of Preparation 22 from 2-amino-5-chloro-3-pyridinol and methyl 3-(bromomethyl)benzoate to give a tan solid:

¹H (δ, CDCl₃, 400 MHz) 3.9 (3H, s), 4.7 (2H, br s), 5.1 (2H, s), 6.95 (1H, s), 7.5 (1H, t), 7.6 (1H, d), 7.65 (1H, s), 8.05 (1H, d), 8.1 (1H, s);

LRMS 293, 295 (MH); 585, 587 (M₂H);

m. pt. 148-149.5° C.;

El. Anal.—Found: C, 57.08; H, 4.41; N, 9.42. Calcd for C₁₄H₁₃ClN₂O₃: C, 57.44, H, 4.48; N, 9.57.

### Preparation 24: 5-Chloro-3-(phenoxymethyl)-2-pyridinamine

Sodium hydride (80% in oil, 124 mg, 4.1 mmol) was added portionwise to a solution of phenol (290 mg, 3.1 mmol) in anhydrous THF (15 ml). 5-Chloro-3-(chloromethyl)-2-pyridinamine.HCl (R. Herbert, D. G. Wibberley, J. Chem. Soc., 1969, 1504) (300 mg, 1.4 mmol) was then added and the reaction stirred at 50° C. for 3 h. After removal of THF in vacuo, the residue was partioned between diethyl ether and 1N NaOH. The aqueous phase was removed, extracted with diethyl ether and the combined organics washed with saturated brine, dried over MgSO₄ and concentrated to an oil which upon triturating with hexane gave the title compound as a white solid (265 mg, 1.1 mmol):

¹H (δ, CDCl₃, 300 MHz) 4.85 (2H, br s), 4.9 (2H, s), 6.9-7.05 (3H, m), 7.25-7.35 (2H, m), 7.4 (1H, s), 8.05 (1H, s);

LRMS 235, 237 (MH);

### Preparation 25: (2-Amino-3,5-dichloro-4-pyridinyl)methanol

To the hydrochloride salt of (2-amino-4-pyridinyl)methanol (J. M. Balkovec, M. J. Szymonifka, J. V. Heck, R. W. Ratcliffe; J. Antibiotics, 1991, 44, 1172) (3.2 g, 20 mmol) in conc HCl (22 ml) at 75-80° C. was added, over 30 mins, hydrogen peroxide (15% aq., 19.6 ml). After stirring at 80° C. for a further 3 h, the reaction mixture was cooled in an ice bath and the resultant yellow solid was removed by filtration, triturated with diisopropylether and diethyl ether to give the title compound as the hydrochloride salt (3.3 g, 14.3 mmol):

¹H (δ, d₆-DMSO, 300 MHz) 4.55 (2H, s), 8.0 (1H, s);

LRMS 193, 195, 197 (MH);

M. Pt. 218 ° C. dec.;

El. Anal.—Found: C, 31.36; H, 3.05; N, 11.97. Calcd for C₆H₆Cl₂N₂O.HCl: C, 31.40; H, 3.07; N, 12.21.

### Preparation 26: 3,5-Dichloro-4-(chloromethyl)-2-pyridinamine

(2-Amino-3,5-dichloro-4-pyridinyl)methanol.HCl (2.2 g, 9.6 mmol) was stirred in thionyl chloride (5 ml) for 16 h at RT. The heterogeneous mixture was diluted with toluene, and the white solid removed by filtration, washed with diethyl ether and dried to give the title compound as the hydrochloride salt (2.27 g, 9.2 mmol):

¹H (δ, d₆-DMSO, 300 MHz) 4.75 (2H, s), 8.05 (1H, s);

LRMS 211, 213, 215, 217 (MH);

m. pt. 208-210° C.;

El. Anal.—Found: C, 28.85; H, 2.48; N, 11.13. Calcd for C₆H₅Cl₃N₂.HCl: C, 29.06; H, 2.44; N, 11.30.

### Preparation 27: 3,5-Dichloro-4-(phenoxymethyl)-2-pyridinamine

Sodium phenoxide was prepared by the reaction of phenol (250 mg, 2.7 mmol) and sodium hydride (60% in oil, 106 mg, 2.7 mmol) in dry THF (15 ml) at RT. The solvent was removed in vacuo and replaced with dry DMF (10 ml), 3,5-dichloro-4-(chloromethyl)-2-pyridinamine (300 mg, 1.2 mmol) was added and the mixture heated to 60° C. for 2.5 h. After reaction mixture was diluted with water (15 ml) and extracted with diethyl ether (4×15 ml). The combined ethereal extracts were washed with water and saturated brine, dried over MgSO₄, then concentrated to a solid. This was crystallised from methylene chloride and hexane to give the title compound as a white solid (219 mg+2^{nd} crop of 45 mg, 1.0 mmol):

¹H (δ, CDCl₃, 300 MHz) 5.0 (2H, br s), 5.2 (2H, s), 6.95-7.05 (3H, m), 7.25-7.35 (2H, m), 8.05 (1H, s);

LRMS 269, 271, 273 (MH);

m. pt. 116-8° C.;

El. Anal.—Found: C, 53.10; H, 3.68; N, 10.33. Calcd for C₁₂H₁₀Cl₂N₂O+0.1 water: C, 53.20; H, 3.79; N, 10.34.

### Preparation 28: N-[(2-Amino-3,5-dichloro-4-pyridinyl)methyl]-N-benzyl-N-methylamine

3,5-Dichloro-4-(chloromethyl)-2-pyridinamine.HCl (300 mg, 1.2 mmol) was stirred in N-benzylmethylamine (3 ml) at RT for 48 h afterwhich the reaction mixture was diluted with water to give an oily precipitate. The supernatent liquor was removed, fresh water was added and again the aqueous layer removed. After trituration with hexane, the solid was dissolved in methylene chloride, dried over MgSO₄, and finally crystallised from methylene chloride-hexane to give the title compound as a fluffy white solid (190 mg, 0.6 mmol):

¹H (δ, CDCl₃, 400 MHz) 2.15 (3H, s), 3.6 (2H, s), 3.75 (2H, s), 4.85 (2H, br s), 7.2-7.3 (5H, m), 7.9 (1H, s);

LRMS 296, 298, 300 (MH);

M. Pt. 124-6° C.;

El. Anal.—Found: C, 56.77; H, 5.10: N, 14.19. Calcd for C₁₄H₁₅Cl₂N₃: C, 56.44; H, 5.04; N, 14.06.

### Preparation 29: 3,5-Dichloro-4-(1-pyrrolidinylmethyl)-2-pyridinamine

The title compound was prepared using the method of preparation 28 using pyrrolidine. White solid:

¹H (δ, CDCl₃, 400 MHz) 1.65-1.8 (4H, m), 2.6-2.7 (4H, m), 3.8 (2H, s), 4.9 (2H, br s), 7.95 (1H, s);

LRMS 246, 248, 250 (MH);

M. Pt. 98-101° C.;

El. Anal.—Found: C, 48.77; H, 5.32; N, 16.98. Calcd for C₁₀H₁₃Cl₂N₃: C, 48.79; H, 5.32; N, 17.07.

### Preparation 30: 1-butyl N-[(t-butoxycarbonyl)amino][(5-methyl-2-pyridinyl)imino]methylcarbamate

To a solution of triethylamine (0.77 ml, 5.5 mmol) and 2-amino-5-picoline (200 mg, 1.8 mmol) in methylene chloride (20 ml) at 0° C. was added 1,3-bis(t-butoxycarbonyl)-2-methyl-2-thiopseudourea (0.59 g, 2.0 mmol) and mercury (II) chloride (0.55 g, 2.0 mmol). The reaction mixture was stirred at RT for 64 h, and the mercury residues filtered off for disposal. The filtrate was chromatographed on silica gel eluting with hexane-EtOAc (95:5 to 90:10) to give t-butyl N-[(t-butoxycarbonyl)amino][(5-methyl-2-pyridinyl)imino]methylcarbamate compound (111 mg, 0.32 mmol):

¹H(δ, CDCl₃, 300 MHz) 1.5 (18H, s), 2.3 (3H, s), 7.5 (1H, br d), 8.1 (1H, d), 8.2 (1H, br s), 10.8 (1H, br s), 11.5 (1H, br s);

LRMS 351 (MH).

Other compounds of formula (IV; P and P¹are both CO₂Bu^{t}) prepared by the same method are listed in Table below.

TABLE 2

| Pre- | | | | | | | |
|---|---|---|---|---|---|---|---|
| pa- | | | | | | | |
| ra- | | | | | Elemental | | |
| tion | R³ | R² | R¹ | Mp ° C. | Analysis | LRMS | ¹H, δ |
| 31 | H | H | Cl | — | — | 371 (MH) | (CDCl₃, 300MHz) 1.5(18H, s), |
| | | | | | | | 7.65(1H, d), 8.25(1H, s), 8.5(1H, d), |
| | | | | | | | 10.9(1H, br s), 11.5(1H, br s) |
| 32 | H | H | Br | — | — | 415, 417 (MH) | (CDCl₃, 300MHz) 1.5(9H, s), 1.5(9H, |
| | | | | | | | s), 7.8(1H, dd), 8.3 1H, d), 8.35(1H, |
| | | | | | | | d), 10.9(1H, br s), 11.45(1H, br s) |
| 33^{(a)} | H | Ph | H | 158-160 | Found: C, | 413 (MH) | (CDCl₃, 400MHz) 1.55(18H, s), |
| | | | | | 63.97; H, 6.82; | | 7.25-7.20(2H, m), 7.4-7.55(3H, m), |
| | | | | | N, 13.64. | | 7.75-7.65(2H, m), 8.35(1H, d), |
| | | | | | Calcd for | | 8.7(1H, br s) |
| | | | | | C₂₂H₂₈N₄O₄: | | |
| | | | | | C, 64.05; H, | | |
| | | | | | 6.84; N, 13.58 | | |
| 34 | H | CONHCH₂Ph | H | — | — | 470 (MH) | (CDCl₃, 400MHz) 1.5(18H, s), 4.5(2H, |
| | | | | | | | d), 6.55(1H, br s), 7.25-7.35(5H, m), |
| | | | | | | | 7.5(1H, d), 8.4(1H, d), 8.7(1H, br s), |
| | | | | | | | 11.0(1H, br s), 11.45(1H, br s) |
| 35 | Cl | H | Cl | — | — | 405, 407, 409 | (DMSO-d₆, 300MHz) 1.5(18H, s), |
| | | | | | | (MH) | 7.4(1H, s), 7.7(1H, s), 8.05(1H, br s), |
| | | | | | | | 11.8(1H, br s) |
| 36^{(b)} | Br | H | Cl | — | — | 449, 451, 453 | (CDCl₃, 300MHz) 1.5(18H, s), 7.9(1H, |
| | | | | | | (MH) | d), 8.2(1H, d), 8.4(1H, br s), 11.75(1H, |
| | | | | | | | br s) |
| 37^{(c)} | Cl | H | Br | — | — | 449, 451, 453 | (CDCl₃, 300MHz) 1.5(18H, s), |
| | | | | | | (MH) | 7.85(1H, d), 8.25(1H, br s) |
| 38 | E- | H | Cl | 180-181 | Found: C, | 497 (MH) | (CDCl₃, 300MHz) 1.5(18H, s), |
| | CH═CH—CO₂Bu^{t} | | | | 55.08; H, 6.63; | | 1.55(9H, s), 6.6(1H, d), 7.8(1H, d), |
| | | | | | N, 11.12. | | 8.2(1H, d), 8.25(1H, d), 10.1(1H, br s), |
| | | | | | Calcd for | | 12.6(1H, br s) |
| | | | | | C₂₃H₃₃ClN₄O₆ + | | |
| | | | | | 0.25 H₂O: C, | | |
| | | | | | 55.09; H, 6.73; | | |
| | | | | | N, 11.17 | | |
| 39 | CH₂CH₂CO₂Bu^{t} | H | Cl | 106-8 | Found: C, | 499, 501 (MH) | (CDCl₃, 300MHz) 1.4(9H, s), 1.5(18H, |
| | | | | | 55.39; H, 7.09; | | s), 2.65(2H, dd), 3.1(2H, dd), 7.5(1H, |
| | | | | | N, 11.16. | | s), 8.1(1H, s) |
| | | | | | Calcd for | | |
| | | | | | C₂₃H₃₅ClN₄O₆: | | |
| | | | | | C, 55.36; H, | | |
| | | | | | 7.07; N, 11.23 | | |
| 40 | E- | H | Cl | 175-7 | Found: C, | 454, 456 (MH) | (CDCl₃, 300MHz) 1.55(18H, s), |
| | CH═CHCONHMe | | | | 52.60; H, 6.15; | | 2.95(3H, d), 6.4-6.5(1H, m), 7.5(1H, d), |
| | | | | | N, 15.18. | | 7.7(1H, d), 8.1(1H, d), 8.25(1H, d), |
| | | | | | Calcd for | | 10.1(1H, br s), 12.8(1H, br s) |
| | | | | | C₂₀H₂₈ClN₅O₅: | | |
| | | | | | C, 52.92; H, | | |
| | | | | | 6.22; N, 15.43 | | |
| 41 | E- | H | Cl | — | Found: C, | 530, 532 (MH) | (CDCl₃, 300MHz) 1.2-1.6(18H, br m), |
| | CH═CHCONHCH₂Ph | | | | 58.57; H, 5.96; | | 4.55(2H, d), 6.6-6.75(1H, m), 7.2- |
| | | | | | N, 13.11. | | 7.35(5H, m), 7.5(1H, d), 7.7(1H, d), |
| | | | | | Calcd for | | 8.15(1H, d), 8.45(1H, d), 10.2(1H, br s), |
| | | | | | C₂₆H₃₂ClN₅O₅. | | 12.9(1H, br s) |
| | | | | | C, 58.92; H, | | |
| | | | | | 6.09; N, 13.21 | | |
| 42 | E- | H | Cl | 172-4 | Found: C, | 524, 526 (MH) | (CDCl₃, 300MHz) 1.5(18H, s), 1.4- |
| | CH═CHCO- | | | | 54.09; H, 6.44; | | 1.8(2H, m), 1.8-2.0(2H, m), 2.0-2.2(1H, |
| | (3- | | | | N, 13.05. | | br m), 3.4-4.0(5H, m), 7.6(1H, d), |
| | hydroxypiperidino) | | | | Calcd for | | 7.75(1H, d), 7.65(1H, d), 8.1(1H, d) |
| | | | | | C₂₄H₃₄ClN₅O₆ + | | |
| | | | | | 0.5 H₂O: C, | | |
| | | | | | 54.08; H, 6.62; | | |
| | | | | | N, 13.14 | | |
| 43 | E- | H | Cl | 166-7 | Found: C, | 554, 556 (MH) | (CDCl₃, 400MHz) 1.5(18H, s), 2.95 & |
| | CH═CHCON | | | | 59.07; H, 6.32; | | 3.2(3H, both s), 4.7 & 4.85(2H, both s), |
| | (Me) | | | | N, 12.69. | | 7.2-7.3(5H, m), 7.7-7.9(2H, m), 8.15- |
| | CH₂Ph | | | | Calcd for | | 8.2(2H, m), 10.1(1H, br s) |
| | | | | | C₂₇H₃₄ClN₅O₅ + | | |
| | | | | | 0.25 H₂O: C, | | |
| | | | | | 59.11; H, 6.34; | | |
| | | | | | N, 12.76 | | |
| 44 | E- | H | Cl | — | — | 510, 512 (MH) | (CDCl₃, 300MHz) 1.5(18H, s), 3.6- |
| | CH═CHCOmorpholino | | | | | | 3.9(8H, m), 7.65(1H, d), 7.7(1H, s), |
| | | | | | | | 8.1(1H, d), 8.2(1H, s), 10.4(1H, br s), |
| | | | | | | | 12.9(1H, br s) |
| 45 | E- | H | Cl | — | — | 490, 492 (MH) | (CDCl₃, 400MHz) 1.55(18H, s), |
| | CH═CHSO₂NHMe | | | | | | 2.85(3H, d), 4.3(1H, q), 7.45(1H, d), |
| | | | | | | | 7.65(1H, d), 8.2(1H, d), 8.55(1H, d), |
| | | | | | | | 10.15(1H, br s), 12.7(1H, br s) |
| 46 | E- | H | Cl | 147-9 | Found: C, | 473 (MH) | (CDCl₃, 300MHz) 1.6(18H, s), 7.2- |
| | CH═CHPh | | | | 60.87; H, 6.16; | | 7.4(4H, m), 7.7(2H, d), 7.9(1H, s), 8.05- |
| | | | | | N, 11.85. | | 8.1(2H, m), 10.05(1H, br s), 12.4(1H, |
| | | | | | Calcd for | | br s) |
| | | | | | C₂₄H₂₉ClN₄O₄: | | |
| | | | | | C, 60.94; H, | | |
| | | | | | 6.18; N, 11.85 | | |
| 47 | E- | H | Cl | — | Found: C, | 503, 505 (MH) | (CDCl₃, 300MHz) 1.5(18H, s), |
| | CH═CH- | | | | 59.12; H, 6.13; | | 3.85(3H, s), 6.9(2H, d), 7.25(1H, d), |
| | (4- | | | | N, 10.97. | | 7.6(2H, d), 7.85(1H, d), 7.9(1H, d), |
| | MeOC₆H₄) | | | | Calcd for | | 8.1(1H, d), 10.0(1H, br s), 12.4(1H, br s) |
| | | | | | C₂₅H₃₁ClN₄O₅ + | | |
| | | | | | 0.25 H₂O: C, | | |
| | | | | | 59.17; H, 6.26; | | |
| | | | | | N, 11.04 | | |
| 48 | E- | H | Cl | — | — | 474, 476 (MH) | (CDCl₃, 300MHz) 1.5(18H, s), 7.1- |
| | CH═CH- | | | | | | 7.2(1H, m), 7.6-7.8(2H, m), 7.8-8.0(2H, |
| | (2- | | | | | | m), 8.15(1H, s), 8.25(1H, d), 8.5- |
| | pyridyl) | | | | | | 8.55(1H, m), 10.1(1H, br s), 12.5(1H, |
| | | | | | | | br s) |
| 49 | E- | H | Cl | 114-116 | Found: C, | 479,481 (MH) | (CDCl_{3,} 300MHz) 1.1-1.9(28H, m), |
| | CH═CH- | | | | 59.86; H, 7.30; | | 2.1-2.3(1H, m), 6.2 & 6.35(1H, both |
| | chexyl | | | | N, 11.52. | | dd), 7.15-7.3(1H, m), 7.7 & 7.75(1H, |
| | | | | | Calcd for | | both s), 8.1 & 8.2(1H, both s), 10.0(1H, |
| | | | | | C₂₄H₃₅ClN₄O₄: | | br s) |
| | | | | | C, 60.17; H, | | |
| | | | | | 7.37; N, 11.70 | | |
| 50 | E- | H | Cl | — | — | 517 (MH) | (CDCl₃, 400MHz) 1.55(18H, s), |
| | CH═CH- | | | | | | 6.0(2H, s), 7.8(1H, d), 7.15(1H, d), 7.2- |
| | (3,4- | | | | | | 7.25(1H, m), 7.3(1H, s), 7.9(1H, s), |
| | methylenedioxyphenyl) | | | | | | 7.95(1H, d), 8.1(1H, s), 10.0(1H, br s), |
| | | | | | | | 12.4(1H, br s) |
| 51 | E- | H | Cl | 173-5 | Found: C, | 498, 500 (MH) | (CDCl₃, 400MHz) 1.55-1.6(18H, m), |
| | CH═CH( | | | | 59.95; H, 5.92; | | 7.35(1H, d), 7.45(1H, dd), 7.5(1H, d), |
| | 3-CN—C₆H₄) | | | | N, 13.62. | | 7.8(1H, d), 7.9(1H, d), 8.05(1H, s), |
| | | | | | Calcd for | | 8.15(1H, d), 8.15(1H, d), 10.05(1H, br |
| | | | | | C₂₅H₂₈ClN₅O₄ + | | s), 12.4(1H, br s) |
| | | | | | 0.1 DIPE^{(f)} + 0.25 | | |
| | | | | | H₂O: C, | | |
| | | | | | 59.97; H, 5.88; | | |
| | | | | | N, 13.66 | | |
| 52 | C═CPh | H | Cl | 144-6 | Found: C, | 471, 473 (MH) | (CDCl₃, 300MHz) 1.5(18H, s), 7.3- |
| | | | | | 61.25; H, 5.80; | | 7.4(3H, m), 7.7-7.9(3H, m), 8.1-8.3(1H, |
| | | | | | N, 11.74. | | m), 12.0(1H, br s) |
| | | | | | Calcd for | | |
| | | | | | C₂₄H₂₇ClN₄O₄: | | |
| | | | | | C, 61.20; H, | | |
| | | | | | 5.78; H, 11.90 | | |
| 53 | OPh | H | Cl | — | — | 463, 465 (MH) | (CDCl₃, 400MHz) 1.5(18H, s), 7.1- |
| | | | | | | | 7.3(4H, m), 7.35-7.45(2H, m), 8.05(1H, |
| | | | | | | | br s), 11-11.5(2H, br m) |
| 54 | OCH₂Ph | H | Cl | — | Found: C, | 477, 479 (MH) | (CDCl₃, 400MHz) 1.5(18H, s), 5.15- |
| | | | | | 57.81; H, 6.18; | | 5.25(2H, m), 7.15-7.25(1H, m), 7.3- |
| | | | | | N, 11.61. | | 7.4(2H, m), 7.5-7.6(2H, m), 7.85-8.0(1H, |
| | | | | | Calcd for | | m), 11.85(1H, br s) |
| | | | | | C₂₃H₂₉ClN₄O₅: | | |
| | | | | | C, 57.92; H, | | |
| | | | | | 6.13; N, 11.75 | | |
| 55 | OCH₂CH₂OH | H | Cl | 128-130 | Found: C, | 431, 433 (MH) | (CDCl₃, 400MHz) 1.5(18H, s), 3.6- |
| | | | | | 49.97; H, 6.34; | | 4.2(4H, br m), 7.15(1H, s), 7.2-7.3(1H, br |
| | | | | | N, 12.71. | | s), 7.95(1H, s), 10.1(1H, br s) |
| | | | | | Calcd for | | |
| | | | | | C₁₈H₂₇ClN₄O₆: | | |
| | | | | | C, 50.17; H, | | |
| | | | | | 6.32; N, 13.00 | | |
| 56 | OCH₂CH₂OMe | H | Cl | 164 | Found: C, | 445, 447 (MH) | (CDCl₃, 300MHz) 1.5(18H, s), 3.5(3H, |
| | | | | | 50.54; H, 6.54; | | s), 3.8-3.9(2H, m), 4.15-4.25(2H, m), |
| | | | | | N, 12.12. | | 7.15(1H, s), 7.9(1H, br s), 11.85(1H, |
| | | | | | Calcd for | | br s) |
| | | | | | C₁₉H₂₉ClN₄O₆ + | | |
| | | | | | 0.25 H₂O: C, | | |
| | | | | | 50.77; H, 6.62; | | |
| | | | | | N, 12.47 | | |
| 57 | OCH₂CONCH₂Ph | H | Cl | 178-180 | Found: C, | 534, 536 (MH) | (CDCl₃, 300MHz) 1.4(9H, s), 1.55(9H, |
| | | | | | 55.94; H, 5.99; | | s), 4.6-4.7(4H, m), 7.1-7.3(6H, m), |
| | | | | | N, 13.01. | | 7.95-8.0(1H, s), 8.75-8.85(1H, m), |
| | | | | | Calcd for | | 10.1(1H, br s), 12.4(1H, br s) |
| | | | | | C₂₅H₃₂ClN₅O₆: | | |
| | | | | | C, 56.23; H, | | |
| | | | | | 6.04; N, 13.12 | | |
| 58 | OCH₂(3- | H | Cl | 137.5-138 | Found: C, | 535, 537 (MH) | (CDCl₃, 300MHz) 1.5(18H, s), 3.9(3H, |
| | CO₂Me— | | | | 56.06; H, 5.86; | | s), 5.15-5.3(2H, br m), 7.2(1H, s), |
| | C₆H₄) | | | | N, 10.33. | | 7.5(1H, t), 7.8-8.1(4H, m), 11.7(1H, br s) |
| | | | | | Calcd for | | |
| | | | | | C₂₃H₃₁ClN₄O₇: | | |
| | | | | | C, 56.13; H, | | |
| | | | | | 5.84; N, 10.47 | | |
| 59 | CH₂OPh | H | Cl | 103-106 | Found: C, | 477, 479 (MH) | (CDCl₃, 400MHz) 1.5(18H, s), |
| | | | | | 58.16; H, 6.32; | | 5.35(2H, s), 6.85-6.95(1H, m), 7.0(2H, |
| | | | | | N, 11.42. | | d), 7.15-7.25(2H, m), 7.8(1H, s), 8.1(1H, |
| | | | | | Calcd for | | s), 10.0(1H, br s), 11.9(1H, br s) |
| | | | | | C₂₃H₂₉ClN₄O₅: | | |
| | | | | | C, 57.92; H, | | |
| | | | | | 6.13; N, 11.75 | | |
| 60^{(d)} | Cl | Cl | Cl | — | Found: C, | 439, 441, 443 | (CDCl₃, 300MHz) 1.5(18H, s), 8.2(1H, |
| | | | | | 44.41; H, 4.98; | (MH) | br s) |
| | | | | | N, 12.18. | | |
| | | | | | Calcd for | | |
| | | | | | C₁₆H₂₁Cl₃N₄O₄ + | | |
| | | | | | 1/3 EtOAc: | | |
| | | | | | C, 44.32; H, | | |
| | | | | | 5.06; N, 12.02 | | |
| 61^{(e)} | Cl | Me | Cl | 106-8 | Found: C, | 419, 421 (MH) | (CDCl₃, 300MHz) 1.5(18H, s), 2.5(3H, |
| | | | | | 48.72, H, 5.77, | | s), 7.2(1H, s), 8.1(1H, br s), 8.3(1H, br |
| | | | | | N; 13.33. | | s) |
| | | | | | Calcd for | | |
| | | | | | C₁₇H₂₄Cl₂N₄O₄: | | |
| | | | | | C, 48.69; H, | | |
| | | | | | 5.77; N, 16.36 | | |
| 62 | Cl | CH₂OPh | Cl | — | — | 511, 513, 515 | (CDCl₃, 300MHz) 1.5(18H, s), |
| | | | | | | (MH) | 5.25(2H, s), 6.9-7.0(3H, m), 7.2-7.35(2H, |
| | | | | | | | m), 8.2-8.4(1H, m), 11.7(2H, br s) |
| 63 | Cl | CH₂NMeCH₂Ph | Cl | 136-7 | Found: C, | 538, 540, 542 | (CDCl₃, 300MHz) 1.5(18H, s), |
| | | | | | 55.74; H, 6.10; | (MH) | 3.15(3H, s), 3.6(2H, s), 3.8 & 3.9(2H, |
| | | | | | N, 12.95. | | both s), 7.2-7.4(6H, m), 8.15 & 8.3(1H, |
| | | | | | Calcd for | | both s), 11.75(1H, br s) |
| | | | | | C₂₅H₃₃Cl₂N₅O₄: | | |
| | | | | | C, 55.76; H, | | |
| | | | | | 6.18; N, 13.01 | | |
| 64 | Cl | CH₂N(CH₂)₄ | Cl | 142-4 | Found: C, | 488, 490, 492 | (CDCl₃, 400MHz) 1.4-2.4(24H, br m), |
| | | | | | 49.96; H, 6.30; | (MH) | 2.4-3.0(2H, br m), 3.6-4.7(2H, br m), |
| | | | | | N, 13.76. | | 8.2-8.4(1H, br m), 10.2-13(2H, br m) |
| | | | | | Calcd for | | |
| | | | | | C₂₁H₃₁Cl₂N₅O₄ + 1 | | |
| | | | | | H₂O: C, | | |
| | | | | | 49.80; H, 6.57; | | |
| | | | | | N, 13.83 | | |

^{(a)}C. Li, L. S. Rittmann, A. S. Tsiftsoglou, K. K. Bhargava, A. C. Sartorelli, J. Med. Chem., 1978, 21, 874
^{(b)}C. W Murtiashaw, R. Breitenbach, S. W. Goldstein, S. L. Pezzullo, G. J. Quallich, R. Sarges, J. Org. Chem., 1992, 57, 1930
^{(c)}G. Mattern, Helv. Chim. Acta. 1977, 60, 2062
^{(d)}K. S. Gudmundsson, J. M. Hinkley, M. S. Brieger, J. C. Drach, L. B. Townsend; Syn. Comm., 1997, 27, 861
^{(e)}T. J. Kress, L. L. Moore, S. M. Costantino, J. Org. Chem., 1976, 41, 93.
^{(f)}DIPE = diisopropylether.

PCS10322 Compounds

As indicated above, suitable inhibitor compounds (agents) for use in the present invention are disclosed in GB patent application No. 9912961 (incorporated herein by reference), U.S. patent application No. 60/169578 (incorporated herein by reference) and PCT patent application No. PCT/IB00/00667 (incorporated herein by reference). It is to be understood that if the following teachings refer to further statements of inventions and preferred aspects then those statements and preferred aspects have to be read in conjunction with the aforementioned statements and preferred aspects—viz pharmaceutical compositions either comprising an iUPA and/or an iMMP and a growth factor (as well as the uses thereof) or comprising an iUPA and an iMMP and an optional growth factor (as well as the uses thereof).

The PCS10322 compounds are substituted α-aminosulphonyl-acetohydroxamic acids which are inhibitors of zinc-dependent metalloprotease enzymes. In particular, the compounds are inhibitors of certain members of the matrix metalloprotease (MMP) family.

According to Aspect A, the PCS10322 compounds have the general formula (I): 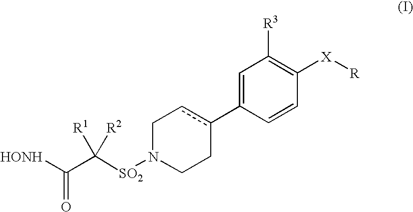
and pharmaceutically-acceptable salts thereof, and solvates thereof,
wherein the dotted line represents an optional bond,
X is a monocyclic aromatic linker moiety selected from phenylene, pyridinylene, pyrazolylene, thiazolylene, thienylene, furylene, pyrimidinylene, pyrazinylene, pyridazinylene, pyrrolylene, oxazolylene, isoxazolylene, oxadiazolylene, thiadiazolylene, imidazolylene, triazolylene, or tetrazolylene;
R is H, C₁₄ alkyl optionally substituted by C₁₋₄ alkoxy, NR⁴R⁵ or OH, or R is C₁₋₄ alkoxy optionally substituted by 1 or 2 substituents selected from (C₁₋₄ alkyl optionally substituted by OH), C₁₋₄ alkoxy, OH and NR⁴R⁵;
R¹ and R² are each independently H, C₁₋₆ alkyl optionally substituted by OH or C₁₋₄ alkoxy; or C₂₋₆ alkenyl;
or R¹ and R² are taken together, with the C atom to which they are attached, to form a 3- to 7-membered ring optionally incorporating a hetero-moiety selected from O, S, SO, SO₂ and NR⁶, and which 3- to 7-membered ring is optionally substituted by one or more OH;
R³ is H, halo, methyl, or methoxy;
R⁴ and R⁵ are each independently H or C, to C₆ alkyl optionally substituted by OH, C₁ to C₄ alkoxy or aryl,
or R⁴ and R⁵ can be taken together with the N atom to which they are attached , to form a 3- to 7-membered ring, optionally incorporating a further hetero- moiety selected from O, S, SO₂ and NR⁷; and
R⁶ and R⁷ are each independently H or C₁ to C₄ alkyl.

According to a further aspect of the invention (“B”), there is provided a compound of formula (I): 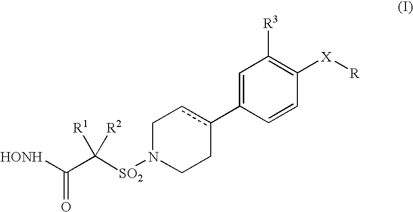
and pharmaceutically-acceptable salts thereof, and solvates thereof,
wherein
the dotted line represents an optional bond;
X is a monocyclic aromatic linker moiety selected from pyrazolylene, thiazolylene, pyrazinylene, pyridazinylene, pyrrolylene, oxazolylene, isoxazolylene, oxadiazolylene, thiadiazolylene, imidazolylene, triazolylene, or tetrazolylene;
R is H, C₁₋₄ alkyl optionally substituted by C₁₋₄ alkoxy or NR⁴R⁵ or OH, or C₁₋₄ alkoxy optionally substituted by 1 or 2 substituents selected from (C₁₋₄ alkyl optionally substituted by OH), C₁₋₄ alkoxy, OH and NR⁴R⁵;
R¹ and R² are each independently H, C₁₋₆ alkyl optionally substituted by OH or C₁₋₄ alkoxy, or C₂₋₆ alkenyl;
or R¹ and R² are taken, together with the C atom to which they are attached, to form a 3- to 7-membered ring optionally incorporating a hetero-moiety selected from O, S, SO, SO₂ and NR⁶, and which 3- to 7-membered ring is optionally substituted by one or more OH;
R³ is H, halo, methyl, or methoxy;
R⁴ and R⁵ are each independently H or C, to C₆ alkyl optionally substituted by OH, C₁ to C₄ alkoxy or aryl,
or R⁴ and R⁵ can be taken together with the N atom to which they are attached , to form a 3- to 7-membered ring, optionally incorporating a further hetero-moiety selected from O, S, SO₂ and NR⁷; and
R⁶ and R⁷ are each independently H or C₁ to C₄ alkyl.

According to a further aspect of the invention (“C”) there is provided a compound of formula (I): 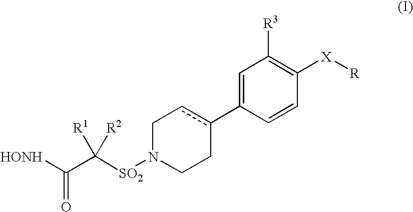
and pharmaceutically-acceptable salts thereof, and solvates thereof,
wherein
the dotted line represents an optional bond;
X is a monocyclic aromatic linker moiety selected from phenylene, pyridinylene, pyrazolylene, thiazolylene, thienylene, furylene, pyrimidinylene, pyrazinylene, pyridazinylene, pyrrolylene, oxazolylene, isoxazolylene, oxadiazolylene, thiadiazolylene, imidazolylene, triazolylene, or tetrazolylene;
R is C₁₋₄ alkyl substituted by NR⁴R⁵, C₁₋₄ alkoxy substituted by NR⁴R⁵, or C₁₋₄ alkoxy substituted by 2 substituents selected from (C₁₋₄ alkyl optionally substituted by OH), C₁₋₄ alkoxy, OH and NR⁴R⁵;
R¹ and R² are each independently H, C₁₋₆ alkyl optionally substituted by OH or C₁₋₄ alkoxy, or C₂₋₆ alkenyl;
or R¹ and R² are taken together, with the C atom to which they are attached, to form a 3- to 7-membered ring optionally incorporating a hetero-moiety selected from O, S, SO, SO₂ and NR⁶, and which 3- to 7-membered ring is optionally substituted by one or more OH;
R³ is H, halo, methyl, or methoxy;
R⁴ and R⁵ are each independently H or C₁ to C₆ alkyl optionally substituted by OH, C₁ to C₄ alkoxy or aryl,
or R⁴ and R⁵ can be taken together with the N atom to which they are attached, to form a 3- to 7-membered ring, optionally incorporating a further hetero-moiety selected from O, S, SO₂ and NR⁷; and
R⁶ and R⁷ are each independently H or C¹ to C₄ alkyl.

According to a further aspect of the invention (“D”) there is provided a compound of formula (I): 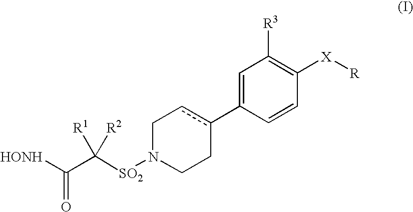
and pharmaceutically-acceptable salts thereof, and solvates thereof,
wherein
the dotted line represents an optional bond,
X is a monocyclic aromatic linker moiety selected from phenylene, pyridinylene, pyrazolylene, thiazolylene, thienylene, furylene, pyrimidinylene, pyrazinylene, pyridazinylene, pyrrolylene, oxazolylene, isoxazolylene, oxadiazolylene, thiadiazolylene, imidazolylene, triazolylene, or tetrazolylene;
R is H, C₁₋₄ alkyl optionally substituted by C₁₋₄ alkoxy, NR⁴R⁵ or OH, or C₁₋₄ alkoxy optionally substituted by 1 or 2 substituents selected from (C₁₋₄ alkyl optionally substituted by OH), C₁₋₄ alkoxy, OH and NR⁴R⁵;
R¹ and R² are each independently C₁₋₆ alkyl substituted by OH;
or R¹ and R² are taken together, with the C atom to which they are attached, to form a 3- to 7-membered ring optionally incorporating a hetero-moiety selected from O, S, SO, SO₂ and NR⁶, and which 3- to 7-membered ring is substituted by one or more OH;
R³ is H, halo, methyl, or methoxy;
R⁴ and R⁵ are each independently H or C₁ to C₆ alkyl optionally substituted by OH, C₁ to C₄ alkoxy or aryl,
or R⁴ and R⁵ can be taken together with the N atom to which they are attached, to form a 3- to 7-membered ring, optionally incorporating a further hetero-moiety selected from O, S, SO₂ and NR⁷; and
R⁶ and R⁷ are each independently H or C₁ to C₄ alkyl.

In all the above definitions A, B, C and D, unless otherwise indicated, alkyl, alkenyl, alkoxy, etc. groups having three or more carbon atoms may be straight chain or branched chain.

For aspects C and D of the invention, X is preferably phenylene, pyridinylene, pyrazolylene or thiazolylene.

For aspects C and D of the invention, X is more preferably 1,3-phenylene, 2,6-pyridinylene, 1,3-pyrazolylene or 2,5-thiazolylene.

For aspect B of the invention X is preferably pyrazolylene or thiazolylene.

For aspect B of the invention X is more preferably 1,3-pyrazolylene or 2,5-thiazolylene.

For aspects B and D of the invention R is preferably H, methoxy, O(CH₂)₂OH, O(CH₂)₂OCH₃, O(CH₂)₂N(CH₃)₂, O(CH₂)₂NHCH₃, O(CH₂)₂NH₂, CH₂NHCH₃, morpholinomethyl, 2-morpholinoethoxy, 2R-2,3-dihydroxy-1-propyloxy, 2S-2,3-dihydroxy-1-propyloxy or 1,3-dihydroxy-2-propyloxy.

For aspects B and D of the invention R is most preferably O(CH₂)₂OH or O(CH₂)₂NH₂.

For aspect C of the invention R is preferably O(CH₂)₂N(CH₃)₂, O(CH₂)₂NHCH₃, O(CH₂)₂NH₂, CH₂NHCH₃, morpholinomethyl, 2-morpholinoethoxy, 2R-2,3-dihydroxy-1-propyloxy, 2S-2,3-dihydroxy-1-propyloxy or 1,3-dihydroxy-2-propyloxy.

For aspect C of the invention R is most preferably O(CH₂)₂NH₂.

For aspects B and C of the invention preferably R¹ and R² are each independently C₁₋₆ alkyl optionally substituted by OH,
or R¹ and R² are taken together, with the C atom to which they are attached, to form a 3- to 7-membered ring optionally incorporating a hetero-moiety selected from O, S, SO, S₂ and NR⁶, and which 3- to 7-membered ring is optionally substituted by one or more OH.

For aspects B and C of the invention more preferably R¹ and R² are each CH₃,
or R¹ and R² are taken together, with the C atom to which they are attached, to form a tetrahydropyran-4-ylidene, piperidin-4-ylidene, 1-methylpiperidin-4-ylidene, or 3,4-dihydroxycyclopentylidene moiety.

For aspects B and C of the invention, yet more preferably R¹ and R² are taken together, with the C atom to which they are attached, to form a tetrahydropyran-4-ylidene, cis-3,4-dihydroxycyclopentylidene, trans-3,4-dihydroxycyclopentylidene or piperidin-4-ylidene moiety.

For aspects B and C of the invention, most preferably R¹ and R² are taken together, with the C atom to which they are attached, to form a tetrahydropyran-4-ylidene, piperidin-4-ylidene, or cis-3,4-dihydroxycyclopentylidene where the hydroxy substituents have a cis-relationship to the hydroxamate moiety.

For aspect D of the invention, R¹ and R² are preferably taken together, with the C atom to which they are attached, to form a 3,4-dihydroxycyclopentylidene moiety.

For aspect D of the invention, most preferably R¹ and R² are taken together, with the C atom to which they are attached, to form a cis-3,4-dihydroxycyclopentylidene group where the hydroxy substituents have a cis-relationship to the hydroxamate moiety.

For aspects A, B, C and D of the invention R³ is preferably methyl.

A preferred group of substances are those selected from the compounds of the Examples and the pharmaceutically acceptable salts and solvates thereof, especially the compounds of Examples 3, 6 and 14 below, and salts and solvates thereof.

In the synthetic methods below, unless otherwise specified, the substituents are as defined above with reference to the compounds of formula (I) as defined above with respect to aspects A, B, C and D.

A compound of formula (I) may be prepared directly from a corresponding acid or acid derivative of formula (II): 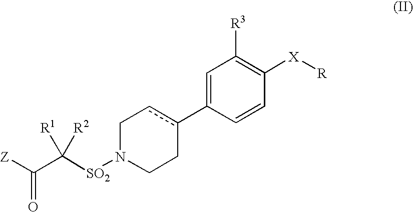
where Z is chloro, bromo, iodo, C₁₋₃ alkyloxy or HO.

When prepared directly from the ester of formula (II), where Z is C₁₋₃ alkyloxy, the reaction may be carried out by treatment of the ester with hydroxylamine, preferably up to a 3-fold excess of hydroxylamine, in a suitable solvent at from about room temperature to about 85° C. The hydroxylamine is conveniently generated in situ from a suitable salt such as its hydrochloride salt by conducting the reaction in the presence of a suitable base such as an alkali metal carbonate or bicarbonate, e.g. potassium carbonate. Preferably the solvent is a mixture of methanol and tetrahydrofuran and the reaction is temperature is from about 65 to 70° C.

Alternatively, the ester (II, where Z is C₁₋₃ alkyloxy) may be converted by conventional hydrolysis to the corresponding carboxylic acid (II, Z is HO) which is then transformed to the required hydroxaric acid of formula (I).

Preferably the hydrolysis of the ester is effected under basic conditions using about 2- to 6-fold excess of an alkali metal hydroxide in aqueous solution, optionally in the presence of a co-solvent, at from about room temperature to about 85° C. Typically the co-solvent is a mixture of methanol and tetrahydrofuran or a mixture of methanol and 1,4-dioxan and the reaction temperature is from about 40 to about 70° C.

The subsequent coupling step may be achieved using conventional amide-bond forming techniques, e.g. via the acyl halide derivative (II, Z is Cl, I or Br) and hydroxylamine hydrochloride in the presence of an excess of a tertiary amine such as triethylamine or pyridine to act as acid-scavenger, optionally in the presence of a catalyst such as 4-dimethylaminopyridine, in a suitable solvent such as dichloromethane, at from about 0° C. to about room temperature. For convenience, pyridine may also be used as the solvent. Such acyl halide substrates are available from the corresponding acid via conventional methods.

In particular, any one of a host of amino acid coupling variations may be used. For example, the acid of formula (II) wherein Z is HO may be activated using a carbodiimide such as 1,3-dicyclohexylcarbodiimide or ]-(3-dimethylaminopropyl)-3-ethylcarbodiimide hydrochloride (often referred to as “water-soluble carbodiimide” or “WSCDI”) optionally in the presence of 1-hydroxybenzotriazole or 1-hydroxy-7-aza-1H-1,2,3-benzotriazole (HOAt) and/or a catalyst such as 4-dimethylaminopyridine, or by using HOAt or a halotrisaminophosphonium salt such as bromotris(pyrrolidino)-phosphonium hexafluorophosphate. Either type of coupling is conducted in a suitable solvent such as dichloromethane, N-methylpyrrolidine (NMP)or dimethylformamide (DMF), optionally in the presence of pyridine or a tertiary amine such as N-methylmorpholine or N-ethyldiisopropylamine (for example when either the hydroxylamine or the activating reagent is presented in the form of an acid addition salt), at from about 0° C. to about room temperature. Typically, from 1.1 to 2.0 molecular equivalents of the activating reagent and from 1.0 to 4.0 molecular equivalents of any tertiary amine present are employed.

Preferred reagents for mediating the coupling reaction are HOAt, WSCDI and O-(7-azabenzotriazol-1-yl)-1,1,3,3-tetramethyluronium hexafluorophosphate (HATU).

Preferably a solution of the acid (II, Z is HO) and N-ethyldiisopropylamine in a suitable solvent such as anhydrous dimethylformamide or anhydrous 1-methylpyrrolidin-2-one, under nitrogen, is treated with up to a 1.5-fold excess of HATU at about room temperature followed, after about 15 to 30 minutes, with up to about a 3-fold excess of hydroxylamine hydrochloride and up to about a 4-fold excess of N-ethyldiisopropylamine, optionally in the same solvent, at the same temperature.

More preferably the acid (II, Z is HO) is reacted with a carbodiimide, HOBt and hydroxylamine hydrochloride in pyridine in a suitable co-solvent such as dichloromethane.

An ester of formula (II, Z is C₁₋₃ alkyloxy) may be prepared from an appropriate amine of formula (III) by sulphonylation with an appropriate compound of formula (IV), wherein R¹⁰ is C₁₋₃ alkyloxy and Z¹ is a leaving group such as Br, I or Cl.

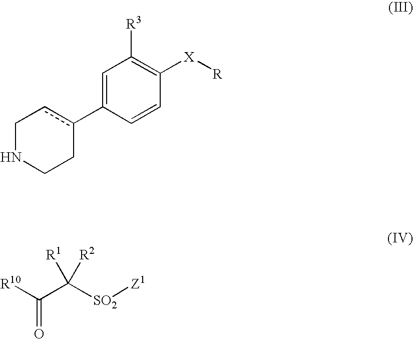

Preferably, Z¹ is chloro.

The reaction may be effected in the presence of an appropriate base in a suitable solvent at from about 0° C. to about room temperature. For example, when both R¹ and R² are hydrogen, an appropriate base is 1,8-diazabicyclo[5.4.0]undec-7-ene and a suitable solvent is dichloromethane. Alternatively, the base can be sodium imidazolide. An alternative method is to make a N-trialkylsilyl dervative of (III), and mix with (IV) at room temperature in tetrahydrofuran (THF) in the presence of a catalytic amount of benzenesulphonic acid (BSA).

Certain esters of formula (II, Z is C₁₋₃ alkyloxy) wherein at least one of R¹ and R² is other than hydrogen may be conveniently obtained from the α-carbanion of an ester of formula (II) wherein at least one of R¹ and R² is hydrogen by conventional C-alkylation procedures using an alkylating agent of formula (VA) or (VB):
R¹Z¹ or R²Z¹ (VA)
Z²(CH₂)_{q}Z³ (VB),
where the (CH₂)_{q} moiety of (VB) optionally incorporates a hetero- moiety selected from O, S, SO, SO₂ and NR⁶, and is optionally substituted by one or more optionally protected OH, and which NR⁶ group may be optionally protected, wherein R¹ and R² are not hydrogen, Z² and Z³ may be the same or different and are suitable leaving groups such as chloro, bromo, iodo, C₁-C₄ alkanesulphonyloxy, trifluoromethanesulphonyloxy or arylsulphonyloxy (e.g. benzenesulphonyloxy or p-toluenesulphonyloxy), and q is 3, 4, 5, 6 or 7. Other conditions are outlined below—sections vii) and x).

Preferably, Z² and Z³ are selected from bromo, iodo and p-toluenesulphonyloxy.

The carbanion may be generated using an appropriate base in a suitable solvent, optionally in the presence of a phase transfer catalyst (PTC). Typical base-solvent combinations may be selected from lithium, sodium or potassium hydride, lithium, sodium or potassium bis(trimethylsilyl)amide, lithium diisopropylamide and butyllithium, potassium carbonate, sodium or potassium t-butoxide, together with toluene, ether, DMSO, 1,2-dimethoxyethane, tetrahydrofuran, 1,4-dioxan, dimethylformamide, N,N-dimethylacetamide, 1-methylpyrrolidin-2-one and any mixture thereof

Preferably the base is sodium hydride and the solvent is dimethylformamide, optionally with tetrahydrofuran as co-solvent, or 1-methylpyrrolidin-2-one. For monoalkylation up to about a 10% excess of base is employed whilst, for dialkylation, from about 2 to about 3 molar equivalents are generally appropriate.

Typically, the carbanion is generated at about room temperature, under nitrogen, and subsequently treated with the required alkylating agent at the same temperature.

Clearly, when dialkylation is required and R¹ and R² are different, the substituents may be introduced in tandem in a “one-pot reaction” or in separate steps.

An amine of formula (III) may be obtained by standard chemical procedures.

Other amines of formula (III), when neither commercially available nor subsequently described, can be obtained either by analogy with the processes described in the Preparations section below or by conventional synthetic procedures, in accordance with standard textbooks on organic chemistry or literature precedent, from readily accessible starting materials using appropriate reagents and reaction conditions.

Another way of making compounds of formula (II) where ZCO is an ester moiety, is via the reaction sequence

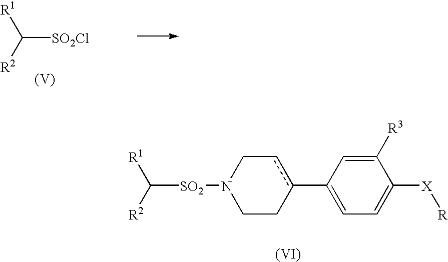

The appropriate sulphonyl chloride (V) is reacted with compound (III—see above) optionally in the presence of a base and in a suitable solvent. The resulting sulphonamide (VI) is reacted with a suitable base such as n-butyllithium, sodium hydride or potassium t-butoxide in a suitable anhydrous non-protic solvent to generate the carbanion α to the sulphonamide moiety, which is then reacted with for example dimethyl carbonate or methyl chloroformate, in suitable conditions, either of which reagent would give the compound (II) where Z is methoxy.

Compounds of formula (I) where R contains a free NH, NH₂ and/or OH group (apart from on the hydroxamic acid moiety) may conveniently be prepared from a corresponding N— or O-protected species (VII below). As such, compounds of formula (VII) where R^{p} is a O— and/or N-protected version of a corresponding compound of the formula (I), are included in the scope of this invention, with regard to aspects A, B, C and D of the invention and the specific compounds of formula (I) mentioned herein, such as those mentioned in the Preparations, as appropriate, below. Suitable protection/deprotection regimes are well known in the art, such as those mentioned in “Protective Groups in Organic Synthesis” by T W Greene and P G M Wuts, John Wiley & Sons Inc (1999).

Suitable OH-protecting groups and regimes include the ethers such as t-butyloxy, tri(C₁₋₄)silyloxy, etc., and esters such as carbonates, sulphonates, C₁₋₄ acylates, etc. mentioned by Greene and Wuts, ibid. chapter 2. Suitable NH-protecting groups and regimes can be found in Greene and Wuts, ibid. chapter 7, and include amides such as “Boc”, amines such as benzyl, etc.

Compounds of formula (VII) may be made by methods described herein and/or by variation of methods described herein which the skilled man will appreciate are routine variations.

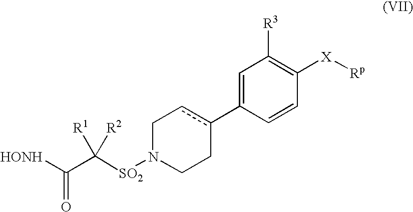

An example of a suitable OH-protecting group is the trimethylsilyl (TMS) group and the protection, reaction, deprotection sequence can be summarised by steps a) to c) below:
a) ClSiMe₃ (1.1 equiv per OH), WSCDI (I. I to 1.2 equiv), HOBT or HOAT (I to 1.1 equiv),
b) NH₂OH.HCl (3 equiv) in DMF/pyridine or CH₂Cl₂/pyridine (3/1 to 1/1) at rt for between 4 and 20 hours.
c) TMS group removed by acid work-up.

Another example of a suitable OH-protecting group is the t-butyl (^{t}Bu) group which can be carried through the synthetic process and removed in the last step of the process. An example of the route is outlined in the scheme below (in relation to the synthesis of the compound of Example 3—via compounds of the Preparations mentioned below).

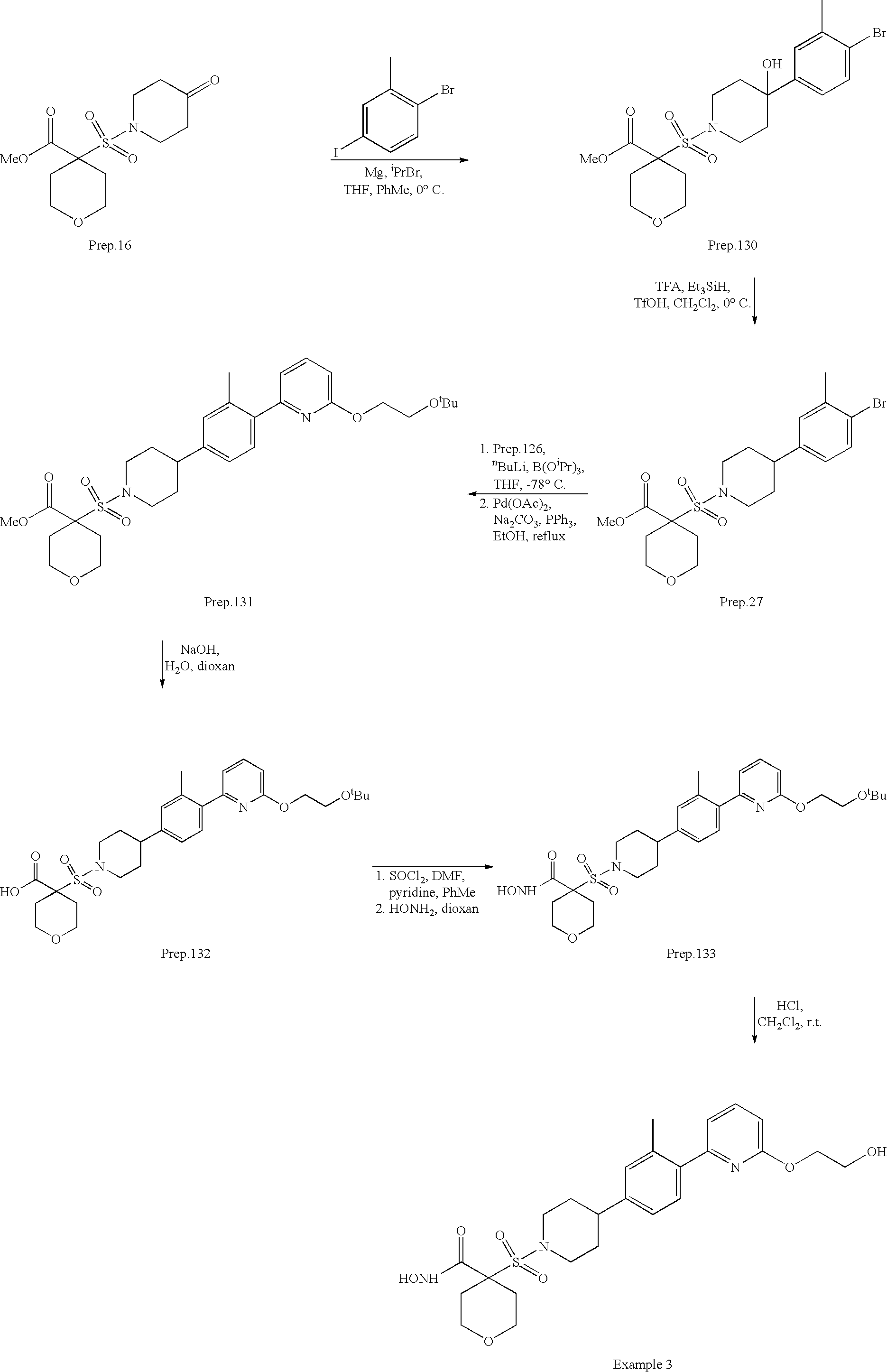

An example of a suitable NH-protecting group is the t-butoxycarbonyl (Boc) group. This group can be introduced in standard ways, such as those delineated in the Examples and Preparations section below. After the hydroxamic acid unit has been introduced, the Boc group can be removed for example by treatment of the N-Boc compound in methanol or dichloromethane saturated with HCl gas, at room temperature for 2 to 4 hours.

Compounds of formula (I) where R¹ and/or R², either independently or together, contain a free NH, NH₂ and/or OH group (apart from on the hydroxamic acid moiety) may conveniently be prepared from a corresponding N— and/or O-protected species (XII below). As such, compounds of formula (XII) where R^{1p} and/or R^{2p} is a O— and/or N-protected version of a corresponding compound of the formula (I), are included in the scope of this invention, with regard to aspects A, B, C and D of the invention and the specific compounds of formula (I) mentioned herein, such as those compounds of formula (XII) mentioned in the Preparations, as appropriate, below. Suitable protection/deprotection regimes are well known in the art, such as those mentioned in “Protective Groups in Organic Synthesis” by T W Greene and P G M Wuts, John Wiley & Sons Inc (1999).

Suitable OH-protecting groups and regimes include the ethers such as t-butyloxy, tri(C₁₋₄)silyloxy, etc., and esters such as carbonates, sulphonates, Con acylates, etc. mentioned by Greene and Wuts, ibid. chapter 2. Suitable NH-protecting groups and regimes can be found in Greene and Wuts, ibid. chapter 7, and include amides such as “Boc”, amines such as benzyl, etc.

Compounds of formula (XII) may be made by methods described herein and/or by variation of 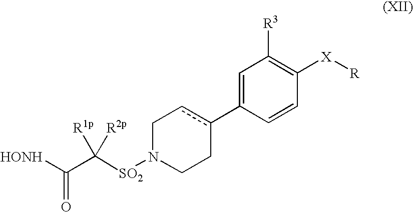
methods described herein which the skilled man will appreciate are routine variations.

An example of a suitable OH-protecting group is the trimethylsilyl (TMS) group and the protection, reaction, deprotection sequence can be summarised by steps a) to c) below:
a) ClSiMe₃(1.1 equiv per OH), WSCDI (1.1 to 1.2 equiv), HOBT or HOAT (1 to 1.1 equiv),
b) NH₂OH.HCl (3 equiv) in DMF/pyridine or CH₂Cl₂/pyridine (3/1 to 1/1) at rt for between 4 and 20 hours.
c) TMS group removed by acid work-up.

Another example of a suitable OH-protecting group is the t-butyl (^{t}Bu) group which can be carried through the synthetic process and removed in the last step of the process. An example of the route is outlined in the scheme below (in relation to the synthesis of the compound of Example 3—via compounds of the Preparations mentioned below).

An example of a suitable NH-protecting group is the t-butoxycarbonyl (Boc) group. This group can be introduced in standard ways, such as those delineated in the Examples and Preparations section below. After the hydroxamic acid unit has been introduced, the Boc group can be removed for example by treatment of the N-Boc compound in methanol or dichloromethane saturated with HCl gas, at room temperature for 2 to 4 hours.

An extension of the above is where the compound of formula (I) contains a free, OH, NH and/or NH₂ group in R¹, R² and R (e.g. some Examples below). In thos case a suitable precursor could be the compound of formula (XIII) below: 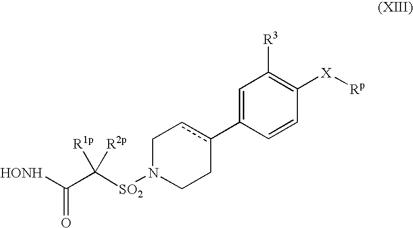
where the substituents are as previously defined

Compounds of formula (I) and appropriate intermediates thereto where R¹ and R² are taken together as 3,4-dihydroxycyclopentylidene can be made via the corresponding intermediacy of a corresponding cyclopent-3-enylidene moiety, viz.:

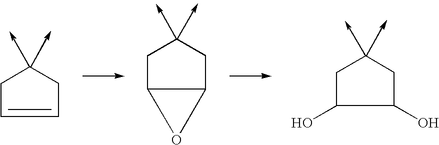

Cyclopentylidene intermediates can be epoxidised to give the corresponding epoxide using standard methods. The epoxide can be reacted in a number of different methods to give the diol product. By suitable choice of reagents, conditions etc., the skilled chemist can make diols with any desired stereochemistry, using well-known methods.

As such, compounds of the formula (VIII) and (IX) below are included in the scope of the invention, with regard to aspects A, B, C and D and also with respect to intermediates to appropriate individual compounds of formula (I) mentioned herein.

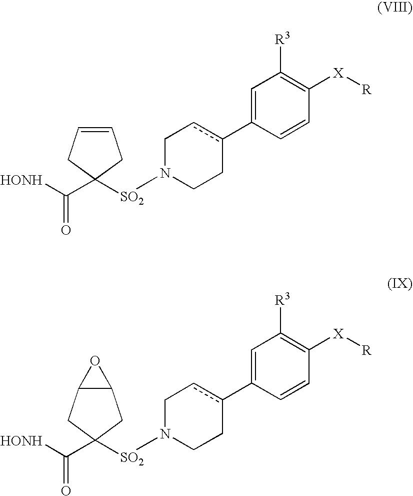

Also included in the invention are intermediates of formula (X) and (XI, where R^{p} is defined as above for compounds of formula (VII) wherein P and P¹ represent standard OH and 1,2-diol protecting groups mentioned in Greene and Wuts, ibid., chapter 2. P and P¹ are preferably taken together and form an acetonide moiety.

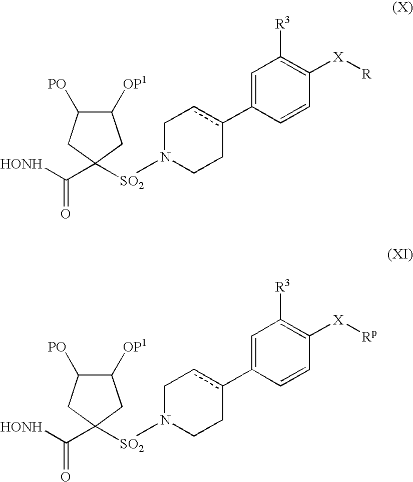

Certain specific compounds of formulae (VIII), (IX), (X) and (XI) are mentioned in the Preparations below.

Preferably the compound is selected from:
N-hydroxy 2-[(4-{4-[6-(2-hydroxyethoxy)pyridin-2-yl]-3-methylphenyl}piperidin-1-yl)sulphonyl]-2-methylpropanamide;
N-hydroxy 2-{[4-(4-{6-[2-(methoxy)ethoxylpyridin-2-yl}-3-methylphenyl)piperidin-1-yl]sulphonyl}-2-methylpropanamide;
N-hydroxy 4-{[4-(4-{6-[2-hydroxyethoxy]pyridin-2-yl}-3-methylphenyl)piperidin-1-yl]sulphonyl}tetrahydro-2H-pyran-4-carboxamide;
N-hydroxy 4-{[4-(4-{6-[(2S)-2,3-dihydroxy-1-propoxy]pyridin-2-yl}-3-methylphenyl)piperidin-1-yl]sulphonyl}tetrahydro-2H-pyran-4-carboxamide;
N-hydroxy 4-{[4-(4-{6-[(2R)-2,3-dihydroxy-1-propoxy]pyridin-2-yl}-3-methylphenyl)piperidin-1-yl]sulphonyl}tetrahydro-2H-pyran-4-carboxamide;
N-hydroxy 4-{[4-(4-{6-[2-hydroxyethoxy]pyridin-2-yl}-3-methylphenyl)piperidin-1-yl]sulphonyl}-piperidine-4-carboxamide dihydrochloride;
N-hydroxy 4-{[4-(4-{6-[2-hydroxyethoxy]pyridin-2-yl}-3-methylphenyl)piperidin-1-yl]sulphonyl}-1-methyl-piperidine-4-carboxamide;
N-hydroxy 2-[4-(4-{3-[(2S)-2,3-dihydroxy-1-propoxy]phenyl}-3-methylphenyl)-piperidin-1-ylsulphonyl]-2-methylpropanamide;
N-hydroxy 4-{4-[4-(3-[(2R)-2,3-dihydroxy-1-propoxy]phenyl)-3-methylphenyl]-piperidin-1-ylsulphonyl}-tetrahydro-(2H)-pyran-4-carboxamide;
N-hydroxy 4-{4-[4-(3-{(2S)-2-hydroxy-2-hydroxymethyl}ethoxyphenyl)-3-methylphenyl]-piperidin-1-ylsulphonyl}-tetrahydro-2H-pyran-4-carboxamide;
N-hydroxy 4-{4-[4-(3-{1,3-dihydroxy-2-propoxyphenyl)-3-methylphenyl]-piperidin-1-ylsulphonyl}-tetrahydro-2H-pyran-4-carboxamide;
N-hydroxy 2-{[4-(4-{3-[2-(methylamino)ethoxy]phenyl}-3-methylphenyl)-piperidin-1-yl]sulphonyl}-2-methylpropanamide hydrochloride;
N-hydroxy 2-[4-(4-{3-(2-aminoethoxy)phenyl}-3-methylphenyl)-piperidin-1-ylsulphonyl]-2-methylpropanamide hydrochloride;
N-hydroxy 4-{[4-(-4-{6-[2-aminoethoxy]pyridin-2-yl}-3-methylphenyl)piperidin-1-yl]sulphonyl}tetrahydro-2H-pyran-4-carboxamide hydrochloride;
N-hydroxy 2-[4-(4-{3-(2-N,N-dimethylaminoethoxy)phenyl}-3-methylphenyl)-piperidin-1-ylsulphonyl]-2-methylpropanamide;
N-hydroxy 4-{[4-(4-{3-(methyl)aminomethyl}-3-methylphenyl)piperidin-1-yl]sulphonyl}tetrahydro-2H-pyran-4-carboxamide hydrochloride;
N-hydroxy 4-{[4-(3-methyl-4-{3-[4-morpholinylmethyl]}phenyl)piperidin-1-yl]sulphonyl}tetrahydro-2H-pyran-4-carboxamide;
N-hydroxy 2-({4-[4-(3-methoxy-1H-pyrazol-1-yl)-3-methylphenyl]piperidin-1-yl}sulphonyl)-2-methylpropanamide;
N-hydroxy 2-[(4-{4-[3-(2-hydroxyethoxy)-1H-pyrazol-1-yl]-3-methylphenyl}piperidin-1-yl)sulphonyl]-2-methylpropanamide;
N-hydroxy 2-methyl-2-({4-[3-methyl-4-(1,3-thiazol-2-yl)phenyl]piperidin-1-yl}sulphonyl)propanamide;
(1α,3α,4α)-N,3,4-trihydroxy-1-[(4-{4-[6-(2-hydroxyethoxy)pyridin-2-yl]-3-methylphenyl}piperidin-1-yl)sulfonyl]cyclopentanecarboxamide;
(1α,3α,4α)-1-({4-[4-(6-ethoxypyridin-2-yl)-3-methylphenyl]piperidin-1-yl}sulfonyl)-N,3,4-trihydroxycyclopentanecarboxamide;
(1α,3β,4β)-1-({4-[4-(6-ethoxypyridin-2-yl)-3-methylphenyl]piperidin-1-yl}sulfonyl)-N,3,4-trihydroxycyclopentanecarboxamide;
(1α,3α,4α)-N,3,4-trihydroxy-1-{4-[4-(3-methoxyphenyl)-3-methylphenyl]piperidin-1-ylsulfonyl}cyclopentanecarboxamide; and
(1α,3β,4β)-N,3,4-trihydroxy-1-{4-[4-(3-methoxyphenyl)-3-methylphenyl]piperidin-1-ylsulfonyl}cyclopentanecarboxamide,
methyl 4-(4-oxo-piperidin-1-ylsulphonyl)tetrahydro-2H-pyran-4-carboxylate;
methyl 4-{[4-(4-bromo-3-methylphenyl)-4-hydroxy-1-piperidin-1-yl]sulfonyl}tetrahydro-2H-pyran-4-carboxylate;
methyl 4-{[4-(4-{6-[2-(tert-butoxy)ethoxy]pyridin-2-yl}-3-methylphenyl)piperidin-1yl]sulfonyl}tetrahydro-2H-pyran-4-carboxylate;
4-{[4-(4-{6-[2-tert-butoxyethoxy]pyridin-2-yl}-3-methylphenyl)piperidin-1yl]sulfonyl}-tetrahydro-2H-pyran-4-carboxylic acid; and
N-hydroxy-4-[(4-{4-[6-(2-tert-butoxyethoxy)pyridin-2-yl]-3-methylphenyl)piperidin-1-yl)sulfonyl]tetrahydro-2H-pyran-4-carboxamide,
N-hydroxy 1-(tert-butoxycarbonyl)-4-{[4-(4-{6-[2-hydroxyethoxy]pyridin-2-yl}-3-methylphenyl)piperidin-1-yl]sulphonyl}-piperidine-4-carboxamide;
1-(tert-butoxycarbonyl)-4-[4-(4-{6-[2-hydroxyethoxy]pyridin-2-yl}-3-methylphenyl)piperidin-1-ylsulphonyl]-piperidine-4-carboxylic acid;
methyl 1-(tert-butoxycarbonyl)-4-{[4-(4-{6-[2-hydroxyethoxy]pyridin-2-yl}-3-methylphenyl)piperidin-1-yl)sulphonyl}-4-piperidinecarboxylate;
methyl 4-{[4-(4-{6-[2-hydroxyethoxy]pyridin-2-yl}-3-methylphenyl)piperidin-1-yl]sulphonyl}-piperidine-4-carboxylate;
methyl 1-benzyl-4-{[4-(4-{6-[2-benzyloxyethoxy]pyridin-2-yl}-3-methylphenyl)piperidin-1-yl]sulphonyl}-piperidin4-carboxylate;
methyl 1-benzyl-4-[4-(4-bromo-3-methylphenyl)piperidin-1-ylsulphonyl]-4-piperidinecarboxylate; and
methyl 2-[4-(4-bromo-3-methylphenyl)piperidin-1-ylsulphonyl]acetate,
N-hydroxy 4-[4-(4-{3-(2-[(N-tert-butoxycarbonyl)amino]ethoxy)phenyl}-3-methylphenyl)-piperidin-1-ylsulphonyl]-tetrahydro-2H-pyran-4-carboxamide;
Preparation 84;
methyl 4-[4-(4-{3-(2-[(tert-butoxycarbonyl)amino]ethoxy)phenyl}-3-methylphenyl)-piperidin-1-ylsulphonyl]-tetrahydro-2H-pyran-4-carboxylate;
methyl 4-[4-(4-{3-(2-aminoethoxy)phenyl}-3-methylphenyl)-piperidin-1-ylsulphonyl]-tetrahydro-2H-pyran-4-carboxylate;
Preparation 61;
methyl 4-[4-(4-{3-(2-oxoethoxy)phenyl}-3-methylphenyl)-piperidin-1-ylsulphonyl]-tetrahydro-2H-pyran-4-carboxylate; and
methyl 4-[4-(4-{3-(2,2-diethoxyethoxy)phenyl}-3-methylphenyl)-piperidin-1-ylsulphonyl]-tetrahydro-2H-pyran-4-carboxylate,
4-[4-(4-{6-[2-hydroxyethoxy]pyridin-2-yl}-3-methylphenyl)piperidin-1-ylsulphonyl]tetrahydro-2H-pyran-4-carboxylic acid;
methyl 4-{[4-(4-{6-[2-hydroxyethoxy]pyridin-2-yl}-3-methylphenyl)piperidin-1-yl]sulphonyl}tetrahydro-2H-pyran-4-carboxylate;
methyl 4-[4-(4-{6-[2-benzyloxy]ethoxypyridin-2-yl}-3-methylphenyl)-1,2,3,6-tetrahydropyridin-1-ylsulphonyl]tetrahydro-2H-pyran-4-carboxylate; and
methyl 4-[4-(4-bromo-3-methylphenyl)-1,2,3,6-tetrahydropyridin-1-ylsulphonyl]tetrahydro-2H-pyran-4-carboxylate,
N-Hydroxy 4-{[4-(4-{6-[2-hydroxyethoxy]pyridin-2-yl}-3-methylphenyl)piperidin-1-yl]sulphonyl}tetrahydro-2H-pyran-4-carboxamide,
N-Hydroxy 4-{[4-(4-{6-[2-hydroxyethoxy]pyridin-2-yl}-3-methylphenyl)piperidin-1-yl]sulphonyl}-piperidine-4-carboxamide,
N-Hydroxy 4-{[4-(-4-{6-[2-aminoethoxy]pyridin-2-yl}-3-methylphenyl)piperidin-1-yl]sulphonyl}tetrahydro-2H-pyran-4-carboxamide,
and pharmaceutically acceptable salts thereof, and solvates thereof.

Moreover, persons skilled in the art will be aware of variations of, and alternatives to, those processes described herein, including in the Examples and Preparations sections, which allow the compounds defined by formula (I) to be obtained, such as carrying out certain bond-forming or functional group interconversion reactions in different sequences.

Examples of the preparation of a number of intermediates and final compounds are outlined in the following synthetic schemes, where the abbreviations used are standard and well-known to the person skilled in the art. Routine variation of these routes can give all the required compounds of the invention.
Route 1 (Pyridyl Alcohols)

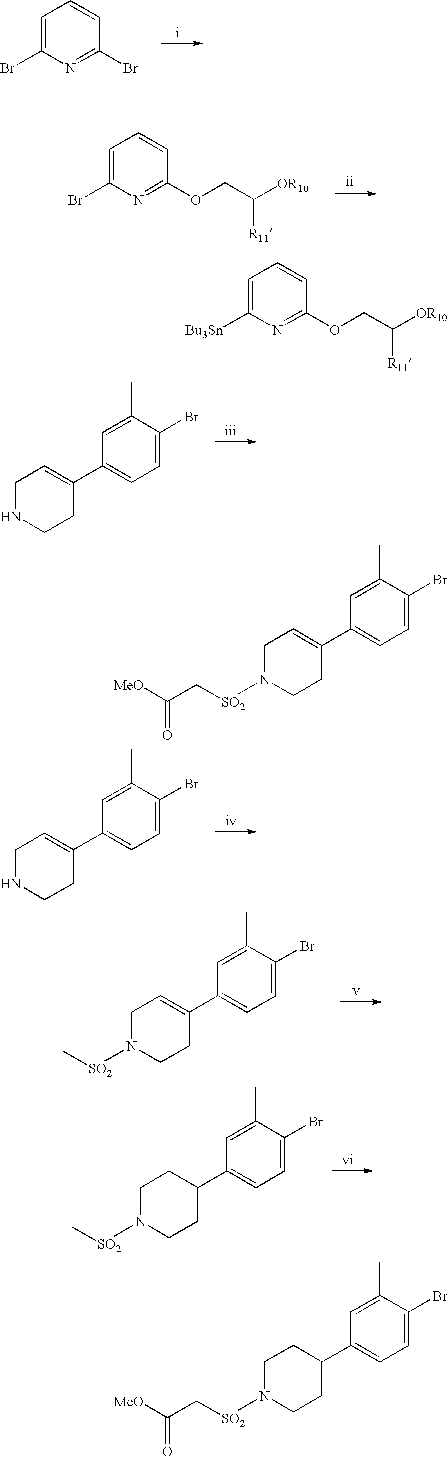

i=NaH (1.1 equiv), HOCH₂CHR11′OR10 (1 equiv) in toluene, reflux for 2 to 5 hours

ii=n-BuLi (1.1 equiv), Bu₃SnCl (1.1 equiv), THF, −70° C. to room temperature.
Or, Pd(PPh₃)₄ (0.01 to 0.05 equiv), [SnMe₃]₂ (1.1 equiv), dioxan, reflux for 2 to 5 hrs.

iii=BSA (0.5 equiv), MeCO₂CH₂SO₂Cl (1.2 equiv), THF, rt for 18 hours.

iv=MeSO₂Cl (1.2 equiv), Et₃N (1.4 equiv), CH₂Cl₂, rt, for an hour.

v=Et₃SiH (3 equiv), CF₃SO₃H (0.1 equiv), TFA:CH₂Cl₂ (1:1), rt, for 1-24 hrs.

vi=NaH (2 equiv), Me₂CO₃ (4 equiv), toluene, reflux for 2 hours.

R10-alcohol protecting group—e.g. benzyl or dioxalane (for diols)

R11′-H or a protected alcohol

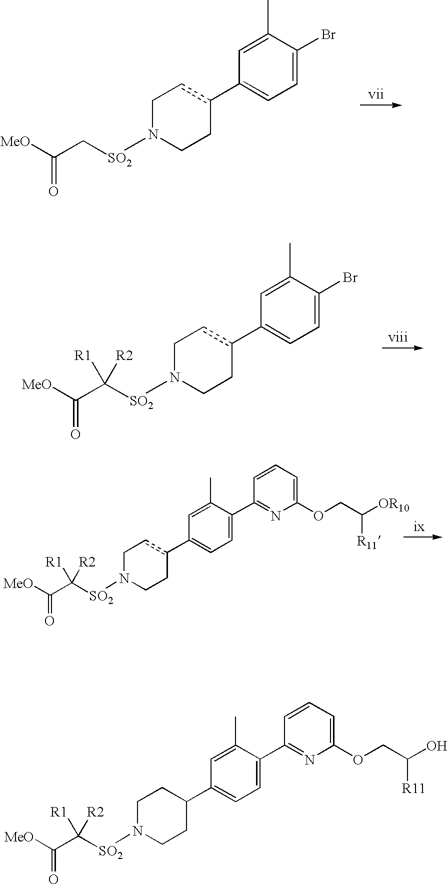

vii=(VB), (1.3 equiv), K₂CO₃ (3 equiv), DMSO, rt, 18-24 hours,
or KOtBu (2.5 equiv), (VA) or (VB) (excess), in THF, rt for 72 hours.

viii=Stille coupling-Pd(PPh₃)₄ (0.05 equiv), stannane (1.5 equiv), toluene, reflux for 4 to 20 hours
OR PdCl₂(PPh₃)₂ (0.05 equiv), stannane (1.1 equiv), THF, reflux for 17 hours.

ix=NH₄⁺HCO₃⁻ (excess) Pd(OH)₂/C, AcOH, MeOH, reflux for 20 hours,
OR 10% Pd/C, in MeOH or EtOH, 3.3 atmospheres, room temperature, for 6 to 17 hours,—both methods also deprotect any benzyl group. (2N HCl, dioxan (3:1), rt, 75 mins at rt—deprotects the dioxalane)
OR Pd(OH)₂/C, NH₄⁺HCO₃⁻ (excess), in MeOH:dioxan (2.5:1), 60° C. for 2 hours.

R11=H or deprotected alcohol

Similarly
when R1R2 when taken together, are a piperidine group: 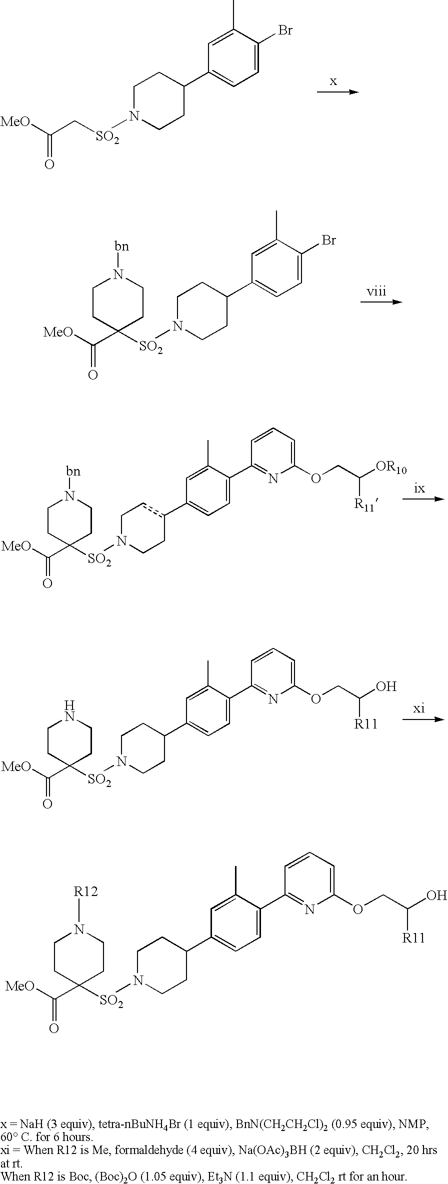
   Route 2 (Phenyl Alcohols)

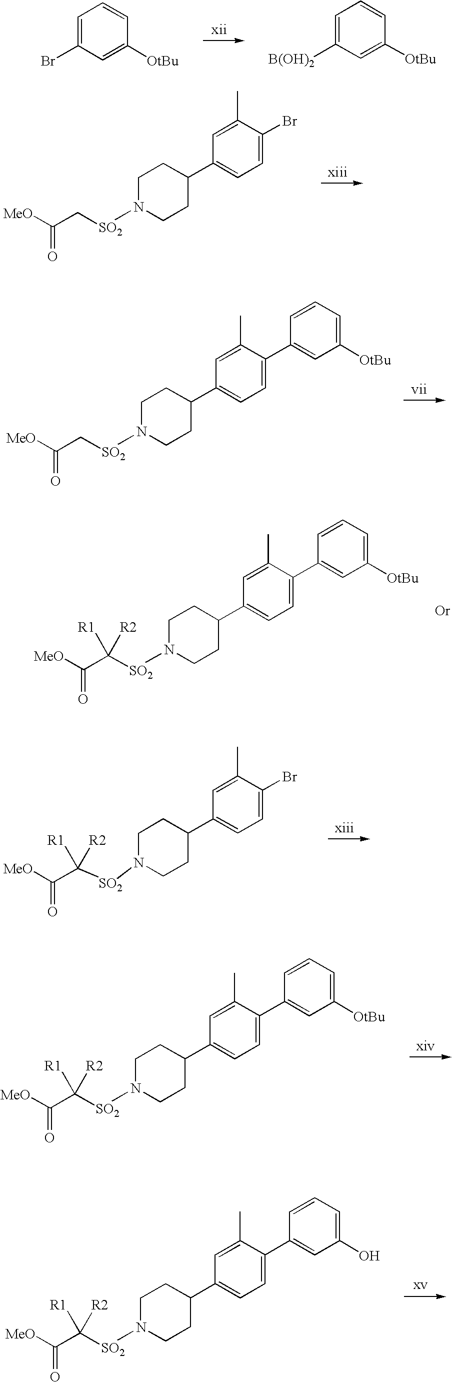

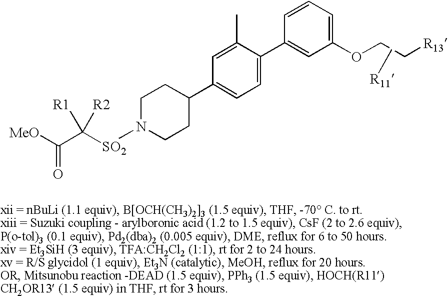

R11 is H or optionally protected alcohol
and R13′ is optionally protected alcohol

For preparation 50 to 51, requires Bn deprotection using the conditions described in ix.
Alternative Route 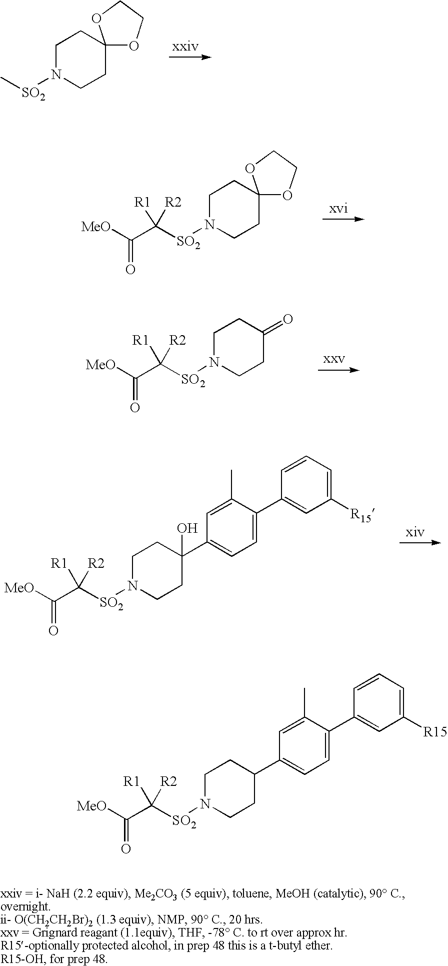
Route 3 (Phenyl Aminoalcohols)

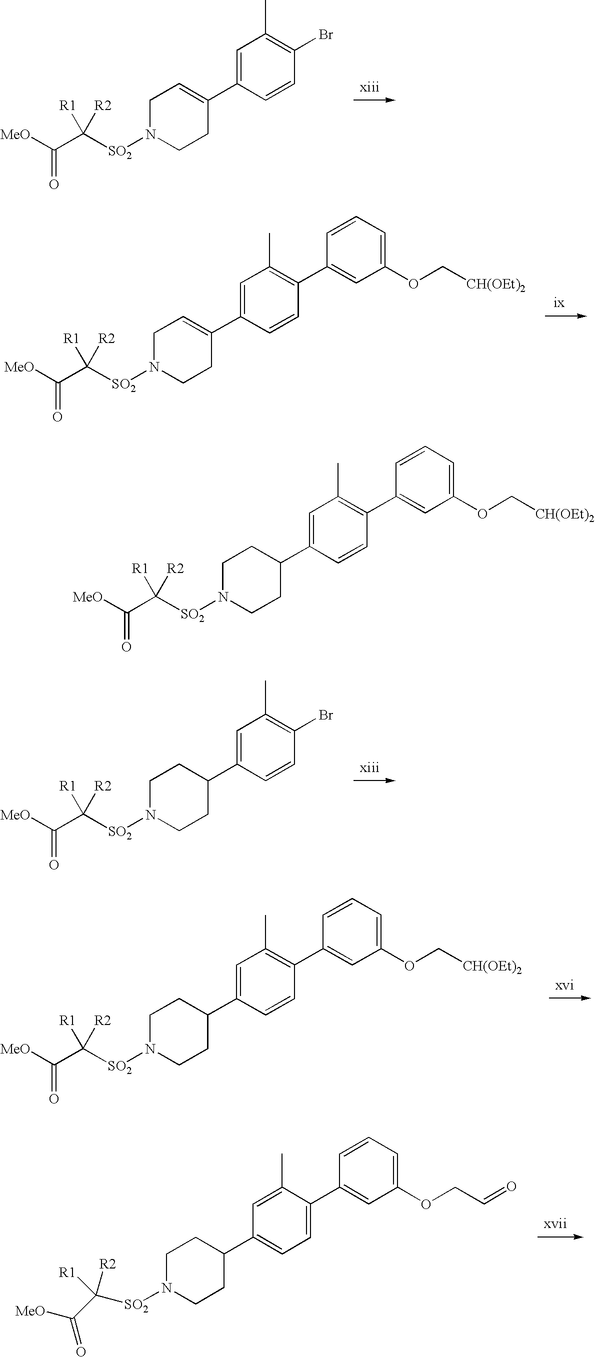

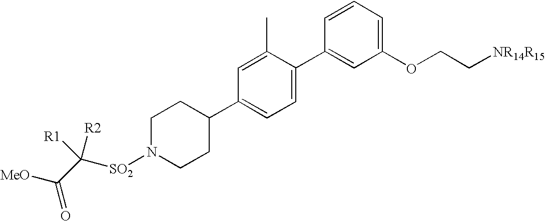

When R15 is a protecting group, eg. benzyl, deprotection, followed by protection using an alternative group eg Boc, can be used as shown below: 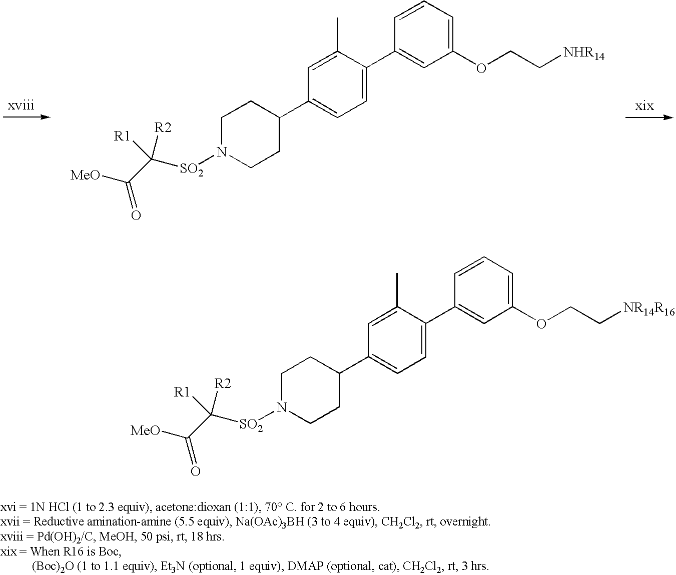
Route 4 (Aminoalkyl Phenyls) 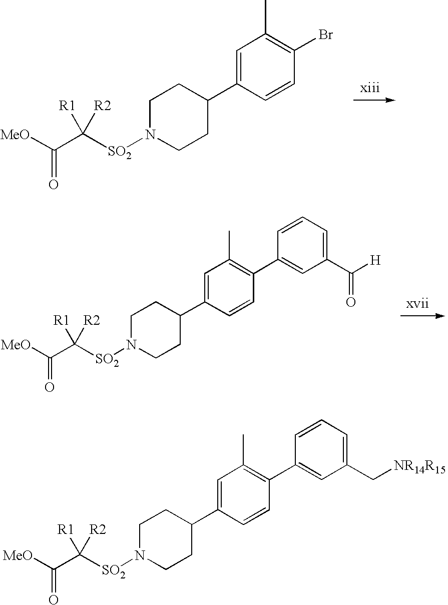
Route 5 (Heterocycles) 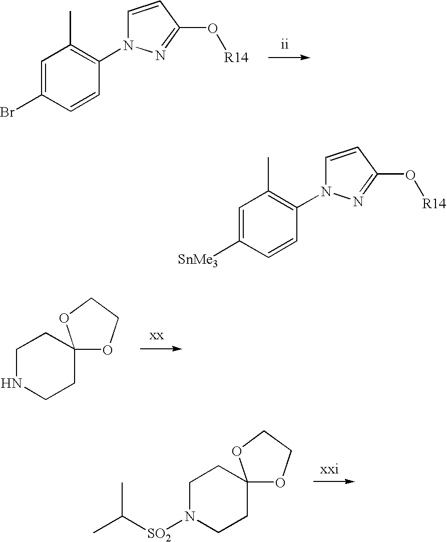 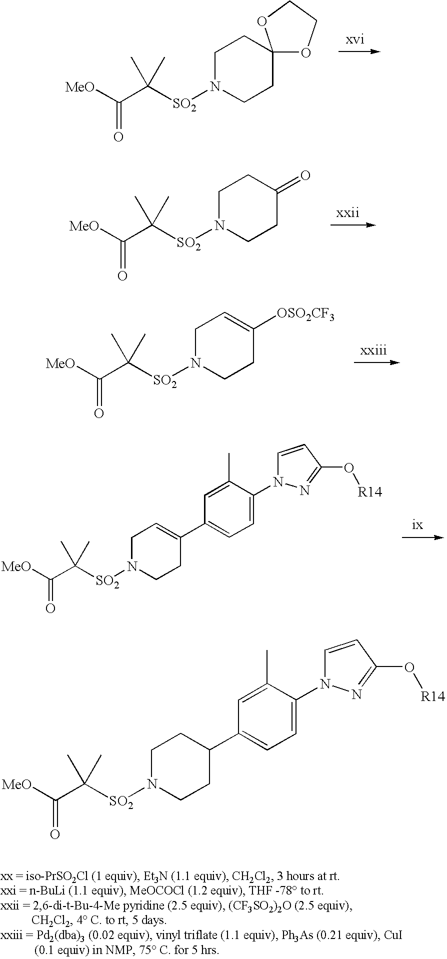
Thiazoles 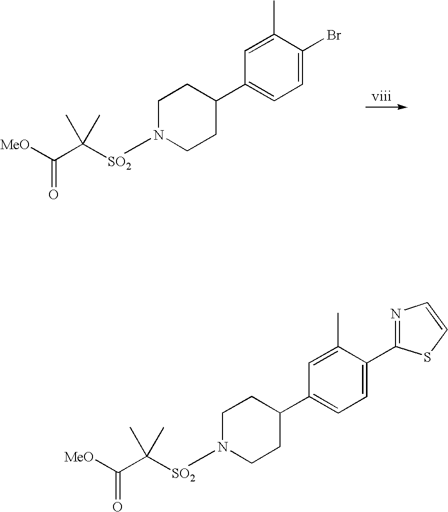
Route 6 (Cyclopentanediols)

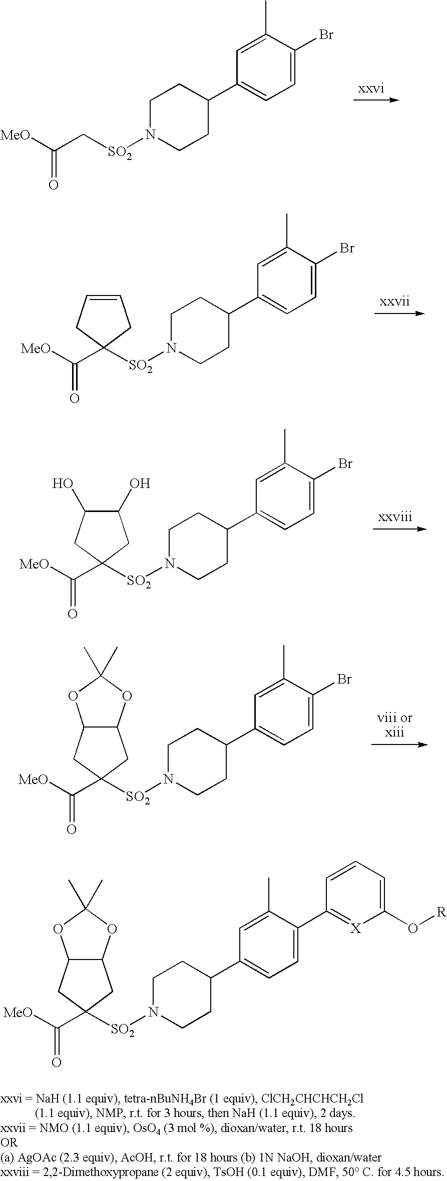

### Examples and Preparations

Room temperature (rt) means 20 to 25° C. Flash chromatography refers to column chromatography on silica gel (Kieselgel 60, 230-400 mesh). Melting points are uncorrected. ¹H Nuclear magnetic resonance (NMR) spectra were recorded using a Bruker AC300, a Varian Unity Inova-300 or a Varian Unity Inova-400 spectrometer and were in all cases consistent with the proposed structures. Characteristic chemical shifts are given in parts-per-million downfield from tetramethylsilane using conventional abbreviations for designation of major peaks: e.g. s, singlet; d, doublet; t, triplet; q, quartet; m, multiplet; br, broad. Mass spectra were recorded using a Finnigan Mat. TSQ 7000 or a Fisons Intruments Trio 1000 mass spectrometer. LRMS means low resolution mass spectrum and the calculated and observed ions quoted refer to the isotopic composition of lowest mass. Hexane refers to a mixture of hexanes (hplc grade) b.p. 65-70° C. Ether refers to diethyl ether. Acetic acid refers to glacial acetic acid. 1-Hydroxy-7-aza-1H-1,2,3-benzotriazole (HOAt), N-[(dimethylamino)-1H-1,2,3-triazolo[4,5-b]pyridin-1-ylmethylene]-N-methylmethaninium hexafluorophosphate N-oxide (HATU) and 7-azabenzotriazol-1-yloxytris(pyrrolidino)phosphonium hexafluorophosphate (PyAOP) were purchased from PerSeptive Biosystems U.K. Ltd. “Me” is methyl, “Bu” is butyl, “Bn” is benzyl. Other abbreviations and terms are used in conjunction with standard chemical practice.

### Example 1

### N-Hydroxy 2-[(4-{4-[6-(2-hydroxyethoxy)pyridin-2-yl]-3-methylphenyl}piperidin-1-yl)sulphonyl]-2-methylpropanamide

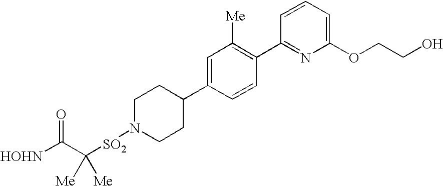

N,N-Dimethylformamide (10 ml) was added to a solution of the acid from preparation 70 (430 mg, 0.93 mmol) in pyridine (5 ml), followed by chlorotrimethylsilane (130 μl, 1.03 mmol) and the solution stirred for 1½ hours. 1-(3-Dimethylaminopropyl)-3-ethylcarbodiimide hydrochloride (215 mg, 1.1 mmol) and 1-hydroxybenzotriazole hydrate (130 mg, 0.93 mmol) were added, and the reaction stirred for a further 2 hours. Hydroxylamine hydrochloride (195 mg, 2.8 mmol) was then added, and the reaction stirred at room temperature overnight. The reaction mixture was acidified to pH 1 using 2N hydrochloric acid, stirred for an hour, and then the pH re-adjusted to pH 4. Water (50 ml) was added, the resulting precipitate filtered, washed with water and dried under vacuum. This solid was purified by column chromatography on silica gel using dichloromethane:methanol:0.88 ammonia (90:10:1) as eluant to afford the title compound as a white solid, (220 mg, 49%).

mp 137-140° C.

¹H nmr (DMSO-d₆, 300 MHz) δ: 1.50 (s, 6H), 1.61 (m, 2H), 1.80 (m, 2H), 2.36 (s, 3H), 2.68 (m, 1H), 3.05 (m, 2H), 3.72 (m, 4H), 4.25 (t, 2H), 4.79 (t, 1H), 6.76 (d, 1H), 7.05 (d, 1H), 7.17 (m, 2H), 7.35 (d, 1H), 7.76 (dd, 1H), 9.00 (s, 1H), 10.55 (s, 1H).

### Example 2

### N-Hydroxy 2-}[4-(4-{6-[2-(methoxy)ethoxy]pyridin-2-yl}-3-methylphenyl)piperidin-1-yl]sulphonyl}-2-methylpropanamide

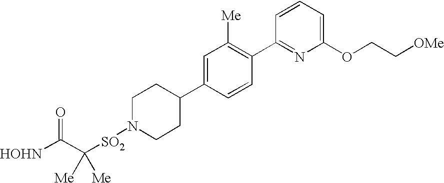

O-(7-Azabenzotriazol-1-yl)-N,N,N′N′-tetramethyluronium hexafluorophosphate (425 mg, 0.95 mmol) and N-ethyldiisopropylamine (150 μl, 0.70 mmol) were added to a solution of the acid from preparation 71 (300 mg, 0.63 mmol) in N,N-dimethylformamide (10 ml), and the solution stirred at room temperature for 30 minutes. Hydroxylamine hydrochloride (158 mg, 1.9 mmol) and additional N-ethyldiisopropylamine (410 μl, 1.9 mmol) were added, and the reacton stirred at room temperature overnight. The reaction mixture was diluted with water (20 ml), and pH 7 buffer solution (20 ml), and then extracted with ethyl acetate (3×30 ml). The combined organic extracts were washed with brine (3×), water (2×), then dried (MgSO₄), filtered and evaporated in vacuo. The residue was triturated with di-isopropyl ether to afford the title compound as an off-white solid, (220 mg, 71%).

mp 134-138° C.

¹H nmr (DMSO-d₆, 300 MHz) δ: 1.48 (s, 6H), 1.61 (m 2H), 1.80 (m, 2H), 2.36 (s, 3H), 2.66 (m, 1H), 3.05 (m, 2H), 3.28 (s, 3H), 3.62 (t, 2H), 3.78 (m, 2H), 4.38 (t, 2H), 6.78 (d, 1H), 7.06 (d, 1H), 7.16 (m, 2H), 7.35 (d, 1H), 7.76 (m, 1H).

Anal. Found: C, 59.65; H, 7.12; N, 7.69. C₂₄H₃₃N₃O₆S;0.2i-Pr₂O requires C, 59.59; H, 7.04; N, 8.04%.

### Example 3

### N-Hydroxy 4-{[4-(4-{6-[2-hydroxyethoxy]pyridin-2-yl-3-methylphenyl)piperidin-1-yl]sulphonyl}tetrahydro-2H-pyran-4-carboxamide

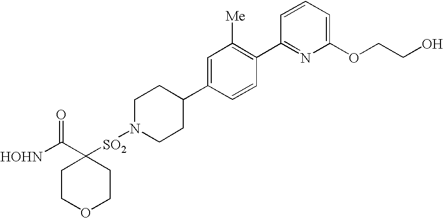

Chlorotrimethylsilane (2.1 ml, 16.46 mmol) was added to a solution of the acid from preparation 72 (7.55 g, 14.96 mmol) in N,N-dimethylformamide (150 ml), and pyridine (150 ml), and the solution stirred at room temperature under a nitrogen atmosphere for 1 hour. 1-(3-Dimethylaminopropyl)-3-ethylcarbodiimide hydrochloride (3.44 g, 17.95 mmol) and 1-hydroxy-7-azabenzotriazole (2.04 g, 14.96 mmol) were added, and stirring was continued for a further 45 minutes. Hydroxylamine hydrochloride (3.12 g, 44.8 mmol) was then added and the reaction stirred at room temperature for 72 hours. The reaction mixture was acidified to pH 2 using hydrochloric acid, stirred for 30 minutes, and the pH then re-adjusted to pH 4 using 1N sodium hydroxide solution. The mixture was extracted with ethyl acetate (3×), the combined organic extracts washed with brine, dried (MgSO₄), filtered and evaporated in vacuo. The residue was purified by column chromatography on silica gel using ethyl acetate as eluant, and recrystallised from methanol/ethyl acetate to afford the title compound as a white solid, (3.75 g, 48%).

mp 193-194° C.

¹H nmr (DMSO-d₆, 400 MHz) δ: 1.61 (m, 2H), 1.79 (m, 2H), 1.92 (m, 2H), 2.36 (m 5H), 2.62 (m, 1H), 3.01 (m, 2H), 3.19 (m, 2H), 3.70 (m, 4H), 3.82 (m, 2H), 4.25 (t, 2H), 4.75 (br, t, 1H), 6.70 (d, 1H), 7.01 (d, 1H), 7.12 (m, 2H), 7.30 (d, 1H), 7.62 (dd, 1H), 9.10 (s, 1H), 10.94 (s, 1H).

LRMS: m/z 520 (M+1)⁺

Anal. Found: C, 57.73; H, 6.39; N, 7.99. C₂₅H₃₃N₃O₇S requires C, 57.79; H, 6.40; N, 8.09%.

Alternative route: Hydrogen chloride gas was bubbled through a solution of the tert-butyl ether from preparation 133 (3.0 g, 5.22 mmol) in anhydrous trifluoroacetic acid (30 ml) and dichloromethane (30 ml) for 10 minutes, then stirred at room temperature overnight. Nitrogen gas was bubbled through the reaction mixture for 1 hour and then 5N NaOH solution until the solution was pH6. The resulting precipitate was cooled to 0° C., filtered and washed with cold water. The resulting solid was dissolved in hot ethyl acetate (500 ml) and the organic layer was washed with water (3×250 ml) and brine (250 ml) and then dried (Na₂SO₄), filtered and concentrated in vacuo. On cooling to 0° C. overnight a solid formed and was filtered, washed with cold ethyl acetate and dried. The title compound was obtained as a beige solid (1.6 g, 60%).

### Example 4

### N-Hydroxy 4-{[4-(4-{6-[(2S)-2,3-dihydroxy--propoxy]pyridin-2-yl}-3-methylphenyl)piperidin-1-yl]sulphonyl}tetrahydro-2H-pyran-4-carboxamide

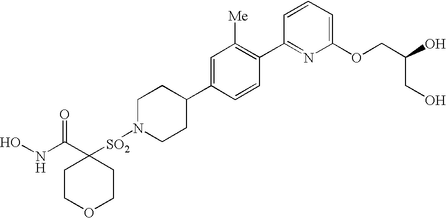

Chlorotrimethylsilane (168 μl, 1.32 mmol) was added to a solution of the acid from preparation 73 (318 mg, 0.60 mmol) in dichloromethane (6 ml), and pyridine (2 ml), and the solution stirred at room temperature under a nitrogen atmosphere for 1 hour. 1-(3-Dimethylaminopropyl)-3-ethylcarbodiimide hydrochloride (138 mg, 0.72 mmol) and 1-hydroxy-7-azabenzotriazole (90 mg, 0.66 mmol) were added, and stirring was continued for a further hour. Hydroxylamine hydrochloride (124 mg, 1.80 mmol) was added and the reaction stirred at room temperature for 2 hours. The reaction mixture was evaporated in vacuo, the residue dissolved in methanol, the solution acidified to pH 1 using hydrochloric acid (2M), then stirred for 10 minutes. The solution was diluted with water, the pH adjusted to 6, and the resulting precipitate filtered and dried. The solid was purified by column chromatography on silica gel using dichloromethane:methanol (90:10) as eluant, and recrystallised from methanol/di-isopropyl ether to give the title compound as a white solid, (200 mg, 60%). ¹H nmr (DMSO-d₆, 400 MHz) δ: 1.61 (m, 2H), 1.79 (m, 2H), 1.92 (m, 2H), 2.36 (m, 5H), 2.63 (m, 1H), 3.03 (m, 2H), 3.08-3.31 (m, 3H), 3.40 (m, 2H), 3.68-3.89 (m, 4H), 4.15 (m, 1H), 4.25 (m, 1H), 4.56 (br, s, 1H), 4.80 (br, s, 1H), 6.75 (d, 1H), 7.04 (d, 1H), 7.14 (m, 2H), 7.34 (d, 1H), 7.75 (m, 1H), (s, 1H), 10.96(s, 1H).

LRMS: m/z 550 (M+1)⁺

Anal. Found: C, 50.70; H, 6.00; N, 6.93. C₂₆H₃₅N₃O₈S;0.6H₂O requires C, 50.97; H, 6.21; N, 6.86%.

### Example 5

### N-Hydroxy 4-{[4-(4-{6-[(2R)-2,3-dihydroxy-1-propoxy]pyridin-2-yl}-3-methylphenyl)piperidin-1-yl]sulphonyl}tetrahydro-2H-pyran-4-carboxamide

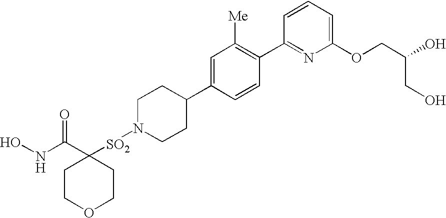

The title compound was prepared from the acid from preparation 74, following the procedure described in example 4. The crude product was purified by crystallisation from ethyl acetate to give an off-white solid (180 mg, 58%).

mp 125-130° C.

¹H nmr (DMSO-d₆, 400 MHz) δ: 1.60 (m, 2H), 1.78 (m, 2H), 1.90 (m, 2H), 2.36 (m, 5H), 2.64 (m, 1H), 3.02 (m, 2H), 3.20 (m, 2H), 3.40 (m, 2H), 3.72 (m, 2H), 3.78 (m, 1H), 3.83 (m, 2H), 4.14 (m, 1H), 4.24 (m, 1H), 4.55 (dd, 1H), 4.80 (d, 1H), 6.75 (d, 1H), 7.03 (d, 1H), 7.15 (m, 2H), 7.32 (d 1H), 7.75 (m, 1H), 9.14 (s, 1H), 10.95 (s, 1H).

LRMS: m/z 572 (M+23)⁺

Anal. Found: C, 55.32; H, 6.57; N, 7.28. C₂₆H₃₅N₃O₈S;H₂O requires C, 55.02; H, 6.57; N, 7.40%.

### Example 6

### N-Hydroxy 4-{[4-(4-{6-[2-hydroxyethoxy]pyridin-2-yl}-3-methylphenyl)piperidin-1-yl]sulphonyl}-piperidine-4-carboxamide dihydrochloride

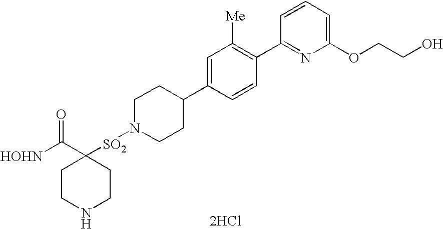

Hydrogen chloride gas was bubbled through an ice-cold solution of the hydroxamic acid from preparation 87 (135 mg, 0.22 mmol) in methanol (20 ml), and the solution was stirred at room temperature. The reaction mixture was evaporated in vacuo, and the residue azeotroped with methanol. The solid was recrystallised from methanol/ether to afford the title compound as a white solid, (88 mg, 64%).

¹H nmr (DMSO-d₆, 400 MHz) δ: 1.63 (m, 2H), 1.80 (m, 2H), 2.07 (m, 2H), 2.35 (s, 3H), 2.56-2.72 (m, 5H), 2.08 (m, 2H), 2.38 (m, 2H), 3.72 (m, 4H), 4.24 (t, 2H), 4.44-4.67 (br, s, 2H), 6.76 (d, 1H), 7.04 (d, 1H), 7.17 (m, 2H), 7.34 (d, 1H), 7.75 (m, 1H), 8.97 (m, 1H), 9.18 (m, 1H).

LRMS: m/z 519 (M+1)⁺

### Example 7

### N-Hydroxy 4-{[4-(4-{6-[2-hydroxyethoxy]pyridin-2-yl}-3-methylphenyl)piperidin-1-yl]sulphonyl}-1-methyl-piperidine-4-carboxamide

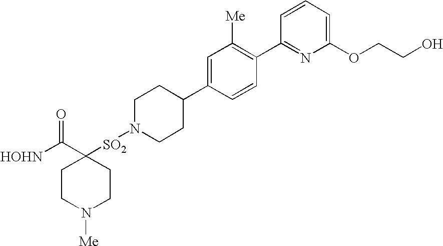

The title compound was prepared from the acid from preparation 75 and hydroxylamine hydrochloride following a similar procedure to that described in example 1. The reaction mixture was acidified to pH 2 using hydrochloric acid, this mixture stirred for 45 minutes, then basified to pH 8 using sodium hydroxide solution (2N). This solution was extracted with ethyl acetate (3×), the combined organic extracts washed with water, then brine, dried (Na₂SO₄), filtered and evaporated in vacuo. The residue was dried at 60° C., under vacuum to afford the title compound (39 mg, 8%).

¹H nmr (DMSO-d₆, 400 MHz) δ: 1.60 (m, 2H), 1.78 (m, 4H), 1.86 (m, 2H), 2.8 (s, 3H), 2.35 (s, 3H), 2.40 (m, 2H), 2.59-2.75 (m, 3H), 3.01 (m, 2H), 3.68 (m, 4H), 4.25 (t, 2H), 4.75 (t, 2H), 6.75 (d, 1H), 7.03 (d, 1H), 7.15 (m, 2H), 7.32 (d, 1H), 7.74 (m, 1H), 9.06 (br, s, 1H), 10.88 (br, s, 1H).

LRMS: m/z 533 (M+1)⁺

Anal. Found: C, 57.91; H, 6.82; N, 10.24. C₂₆H₃₆N₄O₆S;0.3H₂O requires C, 58.04; H, 6.86; N, 10.41%.

### Example 8

### N-Hydroxy 2-[4-(4-{3-[(2S)-2,3-dihydroxy-1-propoxy]phenyl}-3-methylphenyl)piperidin-1-ylsulphonyl]-2-methylpropanamide

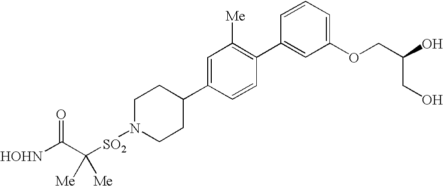

The title compound was prepared from the acid from preparation 77, following a similar procedure to that described in example 3. The crude product was recrystallised from methanol/di-isopropyl ether, to give the desired product (75 mg, 24%) as a white solid. The mother liquors were evaporated in vacuo, and purified by column chromatography on silica gel using an elution gradient of dichloromethane:methanol (98:2 to 95:5) to give an additional (38 mg, 12%) of the desired product.

mp 152-154° C.

¹H nmr (DMSO-d₆, 400MHz) δ: 1.44 (s, 6H), 1.60 (m, 2H), 1.78 (m, 2H), 2.18 (s, 3H), 2.61 (m, 1H), 3.02 (m, 2H), 3.39 (m, 2H), 3.71 (m, 3H), 3.82 (m, 1H), 3.98 (m, 1H), 4.56 (m, 1H), 4.82 (m, 1H), 6.82 (m, 3H), 7.08 (m, 2H), 7.12 (s, 1H), 7.26 (m, 1H), 8.94 (s, 1H), 10.69 (s, 1H).

LRMS: m/z 529 (M+23)⁺

Anal. Found: C, 58.10; H, 6.70; N, 5.09. C₂₅H₃₄N₂O₇S;0.5MeOH requires C, 58.60; H, 6.94; N, 5.36%.

### Example 9

### N-Hydroxy 4-{4-[4-(3-[(2R)-2,3-dihydroxy-1-propoxy]phenyl)-3-methylphenyl]-piperidin-1-ylsulphonyl}-tetrahydro-(2H)-pyran4-carboxamide

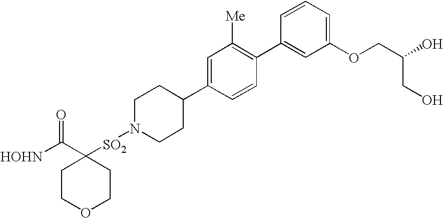

Chlorotrimethylsilane (45 μl, 0.37 mmol) was added to a solution of the acid from preparation 79 (90 mg, 0.17 mmol) in dichloromethane (2 ml), and pyridine (1 ml), and the solution stirred at room temperature under a nitrogen atmosphere for 1 hour. 1-(3-Dimethylaminopropyl)3-ethylcarbodiimide hydrochloride (40 mg, 0.21 mmol) and 1-hydroxy-7-azabenzotriazole (26 mg, 0.19 mmol) were added, and stirring was continued for a further hour. Hydroxylamine hydrochloride (36 mg, 0.51 mmol) was then added and the reaction stirred at room temperature for a further 2 hours. The reaction mixture was diluted with methanol (5 ml), acidified to pH 1 using hydrochloric acid, and the mixture stirred vigorously for an hour. The mixture was extracted with dichloromethane (3×30 ml), the combined organic extracts dried (Na₂SO₄), filtered and evaporated. The residue was purified by column chromatography on silica gel using dichloromethane:methanol (90:10) as eluant to afford the title compound as an off-white solid, (40 mg, 43%).

mp 141-145° C. ¹H nmr (DMSO-d₆, 400 MHz) δ: 1.60 (m, 2H), 1.78 (m, 2H), 1.90 (m, 2H), 2.20 (s, 3H), 2.38 (m, 2H), 2.62 (m, 1H), 3.03 (m, 2H), 3.20 (m, 2H), 3.42 (m, 2H), 3.66-3.90 (m, 6H), 4.01 (m, 1H), 4.60 (m, 1H), 4.90 (m, 1H), 6.84 (m, 3H), 7.14 (m, 3H), 7.30 (m, 1H), 9.18 (s, 1H), 10.98 (1H, s).

LRMS: m/z 571 (M+23)⁺

Anal. Found: C, 59.22; H, 6.80; N, 5.11. C₂₇H₃₆N₂O₈S requires C, 59.11; H, 6.61; N, 5.11%.

### Example 10

### N-Hydroxy 4-{4-[4-(3-{(2S)-2-hydroxy-2-hydroxymethyl}ethoxyphenyl)-3-methylphenyl]-piperidin-1-ylsulphonyl}-tetrahydro-2H-pyran-4-carboxamide

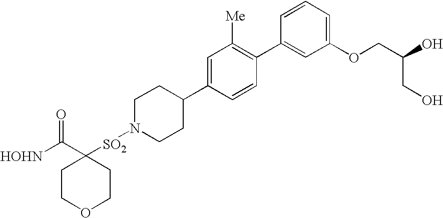

The title compound was prepared, from the acid from preparation 80, following a similar procedure to that described in example 9. The crude product was triturated with methanol/di-isopropyl ether, and the resulting precipitate filtered and dried to afford the title compound as a buff-coloured solid, (158 mg, 45%).

mp 132-134° C.

¹H nmr (DMSO-d₆, 400 MHz) δ: 1.60 (m, 2H), 1.78 (m, 2H), 1.90 (m, 2H), 2.20 (s, 3H), 2.38 (m, 2H), 2.62 (m, 1H), 3.02 (m, 2H), 3.20 (m, 2H), 3.42 (dd, 2H), 3.68-3.90 (m, 6H), 4.00 (m, 1H), 4.60 (t, 1H), 4.97 (d, 1H), 6.81 (m, 2H), 6.90 (m, 1H), 7.08 (s, 2H), 7.15 (s, 1H), 7.29 (dd, 1H), 9.14 (s, 1H), 10.98 (s, 1H).

### Example 11

### N-Hydroxy 4-{4-[4-(3-{1,3-dihydroxy-2-propoxyphenyl)-3-methylphenyl]-piperidin-1-ylsulphonyl}-tetrahydro-2H-pyran-4-carboxamide

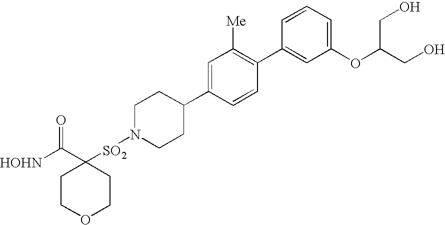

The title compound was obtained (25%) as a white solid, from the acid from preparation 78 and hydroxylamine hydrochloride, using a similar procedure to that described in example 9.

¹H nmr (DMSO-d₆, 400 MHz) δ: 1.60 (m, 2H), 1.79 (m, 2H), 1.90 (m, 2H), 2.20 (s, 3H), 2.39 (m, 2H), 2.62 (m, 1H), 3.02 (m, 2H), 3.20 (m, 2H), 3.57 (m, 4H), 3.70 (m, 2H), 3.84 (m, 2H), 4.24 (m, 1H), 4.78 (m, 2H), 6.82 (d, 1H), 6.90 (m, 2H), 7.14 (m, 3H), 7.28 (m, 1H), 9.18 (br, s, 1H).

LRMS: m/z 570 (M+23)⁺

Anal. Found: C, 56.98; H, 6.65; N, 5.15. C₂₇H₃₆N₂O₈S;H₂O requires C, 57.22; H, 6.76; N, 4.94%.

### Example 12

### N-Hydroxy 2-{[4-(4-{3-[2-(methylamino)ethoxy]phenyl}-3-methylphenyl)-piperidin-1-yl]sulphonyl}-2-methylpropanamide hydrochloride

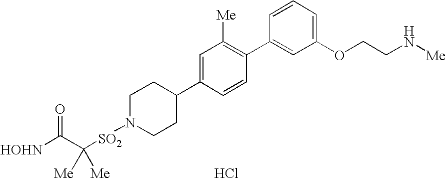

Dichloromethane saturated with hydrogen chloride (12 ml) was added to a solution of the hydroxamic acid from preparation 88 (120 mg, 0.2 mmol) in dichloromethane (1 ml), and the reaction stirred at room temperature for 4 hours. The resulting precipitate was filtered, then washed with, dichloromethane, ether, then dried under vacuum at 60° C., to afford the title compound as a solid, (90 mg, 85%).

mp 180-184° C.

¹H nmr (DMSO-d₆, 400 MHz) δ: 1.44 (s, 6H), 1.60 (m, 2H), 1.78 (m, 2H), 2.18 (s, 3H), 2.59 (m, 3H), 3.02 (m, 2H), 3.28 (m, 2H), 3.72 (m, 2H), 4.23 (t, 2H), 6.90 (m, 3H), 7.08 (s, 2H), 7.16 (s, 1H), 7.34 (m, 1H), 8.83 (br s, 2H), 10.80 (s, 1H).

LRMS: m/z490 (M+1)⁺

Anal. Found: C, 54.25; H, 6.93; N, 7.44. C₂₅H₃₅N₃O₅S;HCl;H₂O;0.1CH₂Cl₂ requires C, 54.56; H, 6.97; N, 7.60%.

### Example 13

### N-Hydroxy 2-[4-(4-{3-(2-aminoethoxy)phenyl}-3-methylphenyl)-piperidin-1-ylsulphonyl]-2-methylpropanamide hydrochloride

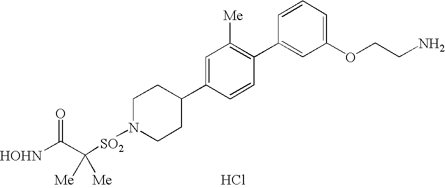

The title compound was obtained as a solid (76%), from the hydroxamic acid from preparation 89, following the procedure described in example 12.

mp 204-206° C.

¹H nmr (DMSO-d₆, 400 MHz) δ: 1.48 (s, 6H), 1.60 (m, 2H), 1.80 (m, 2H), 2.20 (s, 3H), 2.64 (m, 2H), 3.06 (m, 2H), 3.20 (t, 2H), 3.75 (m, 2H), 4.20 (t, 2H), 6.94 (m, 3H), 7.12 (s, 2H), 7.18 (s, 1H), 7.38 (m, 2H), 8.01 (br s, 1H), 8.99 (s, 1H).

LRMS: m/z 476 (M+1)⁺

Anal. Found: C, 55.21; H, 6.74; N, 7.83. C₂₄H₃₃N₃O₅S;HCl;0.5H₂O requires C, 55.32; H, 6.77; N, 8.06%.

### Example 14

### N-Hydroxy 4-{[4-(-4-{6-[2-aminoethoxy]pyridin-2-yl}-3-methylphenyl)piperidin-1-yl]sulphonyl}tetrahydro-2H-pyran-4-carboxamide hydrochloride

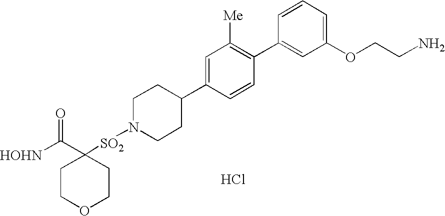

A saturated solution of hydrogen chloride in dichloromethane (250 ml) was added to a solution of the hydroxamic acid from preparation 90 (4.5 g, 7.28 mmol) in dichloromethane (30 ml), and the reaction stirred at room temperature for 3½ hours. The mixture was cooled in an ice-bath, the resulting precipitate filtered off, and washed with dichloromethane, then ether. The solid was then dried under vacuum at 70° C. to afford the title compound (3.1 g, 77%).

mp 208-210° C.

¹H nmr (DMSO-d₆, 400 MHz) δ: 1.60 (m, 2H), 1.78 (m, 2H), 1.90 (m, 2H), 2.19 (s, 3H), 2.38 (m, 2H), 2.62 (m, 1H), 3.02 (m, 2H), 3.19 (m, 6H), 3.70 (m, 2H), 3.83 (m, 2H), 4.18 (t, 2H), 6.92 (m, 2H), 7.06 (s, 2H), 7.17 (s, 1H), 7.35 (m, 1H), 9.12 (s, 1H).

LRMS: m/z 518 (M+1)⁺

### Example 15

### N-Hydroxy 2-[4-(4-{3-(2-N,N-dimethylaminoethoxy)phenyl}-3-methylphenyl)-piperidin-1-ylsulphonyl]-2-methylpropanamide

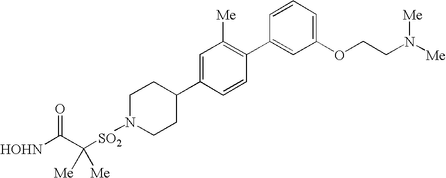

1-(3-Dimethylaminopropyl)-3-ethylcarbodiimide hydrochloride (130mg, 0.68mmol) and 1-hydroxy-7-azabenzotriazole (80 mg, 0.59 mmol) were added to a solution of the acid from preparation 83 (270 mg, 0.55 mmol) in pyridine (6 ml) and dichloromethane (6 ml) under a nitrogen atmosphere, and the suspension stirred for 30 minutes. N,N-dimethylformamide (5 ml), was added, and the reaction warmed to 50° C. to obtain a solution. Hydroxylamine hydrochloride (115 mg, 1.65 mmol) was added and the reaction stirred at room temperature for 18 hours. The reaction mixture was partitioned between ethyl acetate (100 ml) and pH 7 buffer solution (30 ml), and the phases separated. The organic layer was washed with water (2×30 ml), brine (30 ml), dried (Na₂SO₄), filtered and evaporated in vacuo. The residue was azeotroped with toluene (3×), and ethyl acetate (2×), and dried under vacuum at 60° C., to afford the title compound as a solid, (180 mg, 65%).

¹H nmr (DMSO-d₆, 400 MHz) δ: 1.48 (s, 6H), 1.60 (m, 2H), 1.78 (m, 2H), 2.19 (s, 9H), 2.60 (m, 3H), 3.03 (m, 2H), 3.76 (m, 2H), 4.05 (t, 2H), 6.80 (m, 2H), 6.86 (m, 1H), 7.06 (m, 2H), 7.12 (s, 1H), 7.28 (m, 1H).

LRMS: m/z 504 (M+1)⁺

Anal. Found: C, 60.43; H, 7.50; N, 8.08. C₂₆H₃₇N₃O₅S;0.75H₂O requires C, 60.38; H, 7.50; N, 8.12%

### Example 16

### N-Hydroxy 4-{([4-(4-{3-(methyl)aminomethyl}-3-methylphenyl)piperidin-1-yl]sulphonyl}tetrahydro-2H-pyran-4-carboxamide hydrochloride

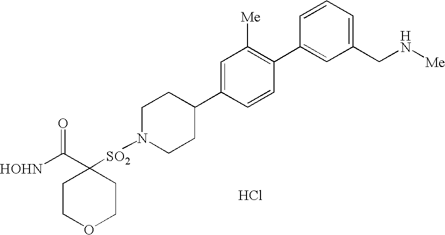

A solution of dichloromethane saturated with hydrogen chloride (20 ml) was added to a solution of the hydroxamic acid from preparation 91 (347 mg, 0.58 mmol) in dichloromethane (10 ml), and the solution stirred at room temperature for 4 hours. The reaction mixture was concentrated in vacuo, and the residue triturated with hot methanol/di-isopropyl ether to give the title compound as a white solid, (202 mg, 64%).

mp 213-214° C.

¹H nmr (DMSO-d₆, 400 MHz) δ: 1.60 (m, 2H), 1.78 (m, 2H), 1.97 (m, 2H), 2.20 (s, 3H), 2.38 (m, 2H), 2.46 (s, 3H), 2.62 (m, 1H), 3.01 (m, 2H), 3.18 (m, 2H), 3.70 (m, 2H), 3.82 (m, 2H), 4.12 (s, 2H), 7.10 (m, 3H), 7.35 (s, 1H), 7.43 (m, 3H), 9.10 (br, s, 1H), 10.92 (s, 1H).

LRMS: m/z 502 (M+1)⁺

Anal. Found: C, 57.16; H, 6.72; N, 7.64. C₂₆H₃₅N₃O₅S;HCl;0.5H₂O reqires C, 57.08; H, 6.82; N, 7.68%.

### Example 17

### N-Hydroxy 4-{([4-(3-methyl-4-{3-[4-morpholinylmethyl]}phenyl)piperidin-1-yl]sulphonyl}tetrahydro-2H-pyran-4-carboxamide

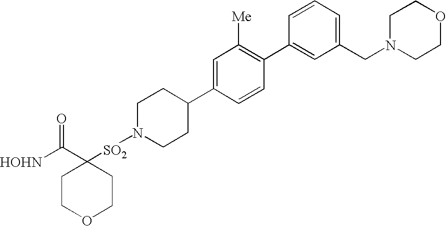

1-(3-Dimethylaminopropyl)-3-ethylcarbodiimide hydrochloride (265 mg, 1.38 mmol) and 1-hydroxy-7-azabenzotriazole (157 mg, 1.15 mmol) were added to a solution of the acid from preparation 86 (625 mg, 1.15 mmol) in pyridine (6 ml) and N,N-dimethylformamide (6 ml) under a nitrogen atmosphere, and the suspension stirred for 1 hour. Hydroxylamine hydrochloride (210 mg, 3.45 mmol) was added and the reaction stirred at room temperature for 18 hours. The reaction mixture was partitioned between ethyl acetate and pH 7 buffer solution, the phases separated, and the aqueous layer extracted with ethyl acetate. The combined organic solutions were washed with water, brine, then dried (MgSO₄), filtered and concentrated in vacuo. The crude product was purified by column chromatography on silica gel using dichloromethane:methanol (95:5) as eluant, and recrystallised from ethyl acetate to give the desired product as a white solid, (398 mg, 62%).

mp 177-179° C.

¹H nmr (DMSO-d₆, 400 MHz) δ: 1.60 (m, 2H), 1.78 (m, 2H), 1.88 (m, 2H), 2.17 (s, 3H), 2.36 (m, 6H), 2.60 (m, 1H), 3.00 (m, 2H), 3.19 (m, 2H), 3.46 (s, 2H), 3.53 (m, 4H), 3.70 (m, 2H), 3.81 (m, 2H), 7.06 (m, 7H), 9.10 (s, 1H), 10.92 (s, 1H).

LRMS: m/z 558 (M+1)⁺

Anal. Found: C, 62.15; H, 7.01; N, 7.40. C₂₉H₃₉N₃O₆S requires C, 62.46; H, 7.05; N, 7.53%.

### Example 18

### N-Hydroxy 2-({4-[4-(3-methoxy-1H-pyrazol-1-yl)-3-methylphenyl]piperidin-1-yl}sulphonyl)-2-methylpropanamide

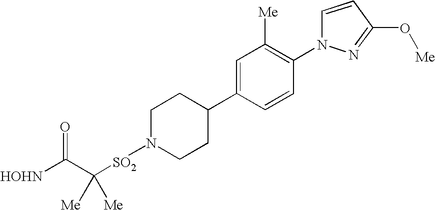

1-(3-Dimethylaminopropyl)-3-ethylcarbodiimide hydrochloride (129 mg, 0.67 mmol) and 1-hydroxy-7-azabenzotriazole (76 mg, 0.56 mmol) were added to a solution of the acid from preparation 103 (235 mg, 0.56 mmol) in pyridine (1.5 ml) and dichloromethane (3 ml) under a nitrogen atmosphere, and the suspension stirred for 30 minutes. Hydroxylamine hydrochloride (78 mg, 1.12 mmol) was added and the reaction stirred at room temperature for 18 hours. The reaction mixture was poured into ethyl acetate (100 ml), washed with pH 7 buffer solution (2×50 ml) then dried (MgSO₄), filtered and evaporated in vacuo. The residual white solid was recrystallised from hot ethyl acetate, to afford the title compound as a white solid, (156 mg, 64%).

mp 172-173° C.

¹H nmr (CD₃OD, 400 MHz) δ: 1.58 (s, 6H), 1.74 (m, 2H), 1.82 (m, 2H), 2.20 (s, 3H), 2.70 (m, 1H), 3.09 (m, 2H), 3.87 (m, 5H), 5.84 (s, 1H), 7.16 (m, 1H), 7.20 (m, 2H), 7.48 (s, 1H).

Anal. Found: C, 55.04; H, 6.42; N, 12.77. C₂₀H₂₈N₄O₅S requires C, 55.03; H, 6.47; N, 12.83%

### Example 19

### N-Hydroxy 2-[(4-{4-[3-(2-hydroxyethoxy)-1H-pyrazol-1-yl]-3-methylphenyl}piperidin-1-yl)sulphonyl]-2-methylpropanamide

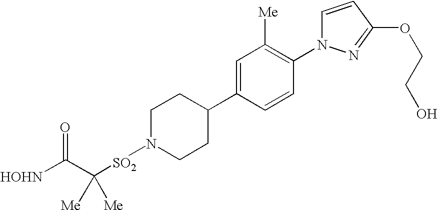

Pyridine (6 ml) was added to a suspension of the acid from preparation 104 (325 mg, 0.72 mmol) in dichloromethane (6 ml), and the solution purged with nitrogen. Chlorotrimethylsilane (858 mg, 0.79 mmol) was added, the solution stirred for an hour, then 1-hydroxy-7-azabenzotriazole (98 mg, 0.72 mmol) was added, followed by 1-(3-dimethylaminopropyl)-3-ethylcarbodiimide hydrochloride (166.8 mg, 0.87 mmol), and the solution was stirred for a further hour. Hydroxylamine hydrochloride (150 mg, 2.16 mmol) was then added and the reaction stirred at room temperature for 17 hours. The reaction was partitioned between ethyl acetate and pH 7 buffer solution, and the pH of the mixture carefully adjusted to 3 using hydrochloric acid (2N). The layers were separated, the organic phase dried (MgSO₄), filtered and evaporated in vacuo, and the residue triturated with ether. The resulting white solid was filtered, then dissolved in a solution of acetic acid (10 ml), water (10 ml), and methanol (10 ml), and this mixture stirred at room temperature for 45 minutes. The solution was poured into pH 7 buffer (300 ml), extracted with ethyl acetate (3×100 ml), and the combined organic extracts dried (MgSO₄), filtered and concentrated in vacuo. The residue was azeotroped with toluene and ethyl acetate, and triturated several times with ether to give the title compound as a white solid, (141 mg, 42%).

¹H nmr (DMSO-d₆, 400 MHz) δ: 1.43 (s, 6H), 1.59 (m, 2H), 1.77 (m, 2H), 2.19 (s, 3H), 2.62 (m, 1H), 3.00 (m, 2H), 3.66 (m, 4H), 4.05 (t, 2H), 4.72 (br, t, 1H), 5.84 (s, 1H), 7.15 (m, 1H), 7.19 (m, 2H), 7.72 (s, 1H), 8.90 (s, 1H), 10.66 (s, 1H).

Anal. Fond: C, 53.85; H, 6.49; N, 11.86. C₂₁H₃₀N₄O₆S requires C, 54.06; H, 6.48; N, 12.01%

### Example 20

### N-Hydroxy 2-methyl-2-({4-[3-methyl-4-(1,3-thiazol-2-yl)phenyl]piperidin-1-yl}sulphonyl)propanamide

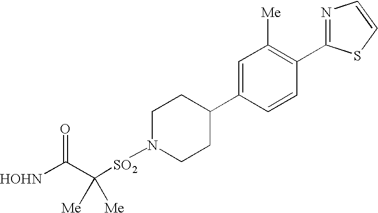

The title compound was prepared from the acid from preparation 105, following the procedure described in example 18. The crude product was crystallised from a minimum volume of methanol to give the desired product as a white solid, (58 mg, 35%).

mp 199-201° C.

¹H nmr (DMSO-d₆, 400 MHz) δ: 1.45 (s, 6H), 1.60 (m, 2H), 2.44 (s, 3H), 2.65 (m, 1H), 3.01 (m, 2H), 3.14 (s, 2H), 3.72 (m, 2H), 7.18 (d, 1H), 7.20 (s, 1H), 7.61 (d, 1H), 7.75 (s, 1H), 7.90 (s, 1H), 8.82 (br, s, 1H), 10.60 (s, 1H).

Anal. Found: C, 53.51; H, 5.92; N, 9.75. C₁₉H₂₅N₃O₄S₂ requires C, 53.88; H, 5.95; N, 9.92%.

### Example 21

### (1α,3α,4α)-N,3,4-trihydroxy-1-[(4-{4-[6-(2-hydroxyethoxy)pyridin-2-yl]-3-methylphenyl}piperidin-1-yl)sulfonyl]cyclopentanecarboxamide

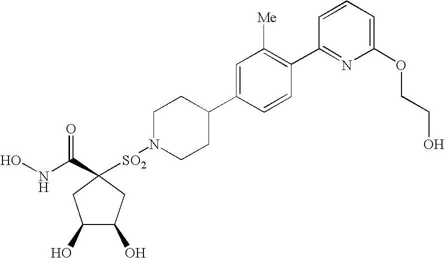

Hydrogen chloride gas was bubbled through a solution of the tert-butyl ether from preparation 121 (260 mg, 0.412 mmol) in trifluoroacetic acid (10 ml) and dichloromethane (10 ml) for 5 minutes, and the reaction was stirred for 5½ hours at ambient temperature. The reaction mixture was evaporated in vacuo and the resulting oil azeotroped with toluene (×2) before partitioning between ethyl acetate (50 ml) and pH7 phosphate buffer solution (40 ml). The organic layer was separated and the aqueous layer was extracted with ethyl acetate (2×50 ml). The combined organic extracts were dried (Na₂SO₄), filtered and evaporated in vacuo. The resulting solid, which contained some of the starting compound, was resubmitted to the reaction conditions. After 5 hours at ambient temperature nitrogen gas was bubbled through the reaction mixture for 15 minutes. The reaction mixture was then evaporated in vacuo and the resulting oil azeotroped with toluene (×2) before partitioning between ethyl acetate (50 ml) and pH7 phosphate buffer solution (40 ml). The organic layer was separated and the aqueous layer extracted with ethyl acetate (2×50 ml). The combined organic extracts were dried (Na₂SO₄), filtered and evaporated in vacuo. The resulting solid was purified by column chromatography on silica gel using dichloromethane/methanol (98:2 to 93:7) as eluant. The title compound was isolated as a white solid (30 mg, 15%).

¹H nmr (DMSO-d₆, 400 MHz) δ: 1.59 (m, 2H), 1.76 (m, 2H), 2.22 (m, 2H), 2.32 (s, 3H), 2.39 (m, 2H), 2.60 (m, 1H), 2.99 (t, 2H), 3.64 (m, 4H), 3.90 (s, 2H), 4.23 (m, 2H), 4.54 (s, 2H), 4.75 (t, 1H), 6.72 (d, 1H), 7.03 (d, 1H), 7.15 (m, 2H), 7.31 (d, 1H), 7.73 (t, 1H), 8.95 (s, 1H), 10.69 (s, 1H).

LRMS: m/z 536 (M+1)⁺.

mp 215-218° C.

Anal. Found: C, 49.73; H, 5.67; N, 6.45. C₂₅H₃₃N₃O₈S;TFA, 0.5MeOH requires C, 49.62; H, 5.45; N, 6.31%.

### Example 22

### (1α,3α,4α)-1-({4-[4-(6-ethoxypyridin-2-yl)-3-methylphenyl]piperidin-1-yl}sulfonyl)-N,3,4-trihydroxycyclopentanecarboxamide

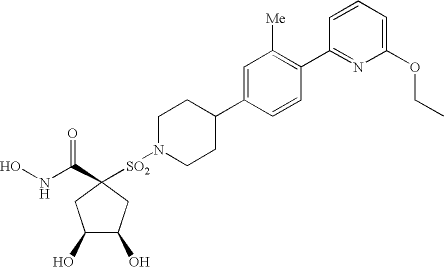

2N Hydrochloric acid (2 ml) was added to a solution of the dioxolane from preparation 122 in dioxan (2 ml) and tetrahydrofuran (2 ml) and the reaction mixture was stirred at ambient temperature for 18 hours. The reaction mixture was evaporated in vacuo and the resulting solid partitioned between pH7 phosphate buffer solution (20 ml) and ethyl acetate (20 ml). The aqueous layer was extracted with ethyl acetate (2×20 ml) and the combined organic extracts were dried (Na₂SO₄) filtered and concentrated in vacuo. The resulting solid was recrystalised from ethyl acetate to afford the title compound as a white solid (95 mg, 70%).

¹H nmr (DMSO-d₆, 400 MHz) δ: 1.25 (t, 3H), 1.58 (m, 2H), 1.76 (m, 2H), 2.22 (m, 2H), 2.35 (s, 3H), 2.38 (m, 2H), 2.60 (m, 1H), 2.99 (t, 2H), 3.66 (d, 2H), 3.85 (s, 2H), 4.25 (q, 2H), 4.61 (s, 2H), 6.71 (d, 1H), 7.03 (d, 1H), 7.12 (m, 2H), 7.31 (d, 1H), 7.72 (t, 1H), 9.00 (s, 1H), 10.78 (s, 1H).

LRMS: m/z 520 (M+1)⁺.

mp 204-205° C.

Anal. Found: C, 57.42; H, 6.36; N, 7.98. C₂₅H₃₃N₃O₇S; 0.25 H₂O requires C, 57.29; H, 6.44; N, 8.02%

### Example 23

### (1α,3β,4β)-1-({4-[4-(6-ethoxypyridin-2-yl)-3-methylphenyl]piperidin-1-yl)sulfonyl)-N,3,4-trihydroxycyclopentanecarboxamide

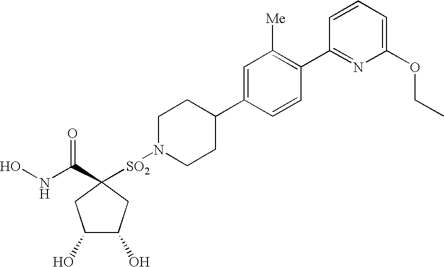

The title compound was prepared from the dioxolane from preparation 123 in a similar procedure to that described in example 22. This afforded the title compound as a white solid (50 mg, 55%).

¹H nmr (DMSO-d₆, 400 MHz) δ: 1.27 (t, 3H), 1.62 (m, 2H), 1.78 (m, 2H), 2.09 (m, 2H), 2.35 (s, 3H), 2.61 (m, 1H), 2.74 (m, 2H), 3.01 (t, 2H), 3.69 (m, 4H), 4.29 (q, 2H), 4.49 (s, 2H), 6.69 (d, 1H), 7.02 (d, 1H), 7.12 (m, 2H), 7.31 (d, 1H), 7.73 (t, 1H), 8.92 (s, 1H), 10.71 (s, 1H).

LRMS: m/z 520 (M+1)⁺.

mp 196-197° C.

Anal. Found: C, 56.83; H, 6.32; N, 7.83. C₂₅H₃₃N₃O₇S; 0.5 H₂O requires C, 56.80; H, 6.48; N, 7.95%.

### Example 24

### (1α,3α,4α)-N,3,4-trihydroxy-1-{4-[4-(3-methoxyphenyl)-3-methylphenyl]piperidin-1-ylsulfonyl}cyclopentanecarboxamide

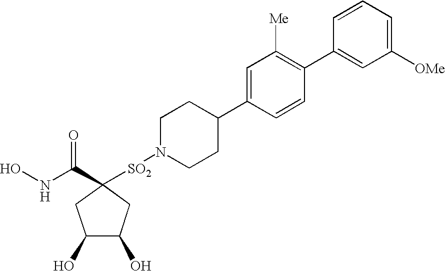

2N Hydrochloric acid (2 ml) was added to a solution of the dioxolane from preparation 124 in dioxan (3 ml) and tetrahydrofuran (2 ml) and the reaction mixture was stirred at ambient temperature for 4 hours. The reaction mixture was evaporated in vacuo and the resulting solid was partitioned between water (20 ml) and ethyl acetate (20 ml). The aqueous layer was extracted with ethyl acetate (2×20 ml) and the combined organic extracts were dried (Na₂SO₄), filtered and concentrated in vacuo. The resulting solid was recrystalised from ethyl acetate to afford the title compound as a white solid (60 mg, 46%).

¹H nmr (DMSO-d₆, 400 MHz) δ: 1.58 (m, 2H), 1.76 (m, 2H), 2.19 (s, 3H), 2.24 (m, 2H), 2.38 (m, 2H), 2.60 (m, 1H), 2.99 (t, 2H), 3.71 (m, 5H), 3.79 (s, 2H), 4.54 (s, 2H), 6.82 (m, 3H), 7.11 (m, 3H), 7.32 (t, 1H), 8.97 (s, 1H), 10.70 (s, 1H).

LRMS: m/z 527 (M+23)⁺.

mp 201-202° C.

Anal. Found: C, 58.85; H, 6.36; N, 5.51. C₂₅H₃₂N₂O₇S; 0.25 H₂O requires C, 58.98; H, 6.43; N, 5.50%.

### Example 25

### (1α,3β,4β)-N,3,4-trihydroxy-1-{4-[4-(3-methoxyphenyl)-3-methylphenyl]piperidin-1-ylsulfonyl}cyclopentanecarboxamide

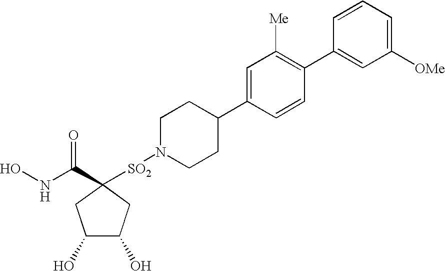

The title compound was prepared from the dioxolane from preparation 125 in a similar procedure to that described in example 24. This afforded the title compound as a white solid (55 mg, 50%).

¹H nmr (DMSO-d₆, 400 MHz) δ: 1.59 (m, 2H), 1.76 (m, 2H), 2.17 (m, 2H), 2.19 (s, 3H), 2.60 (m, 1H), 2.71 (m, 2H), 2.99 (t, 2H), 3.70 (m, 7H), 4.61 (s, 2H), 6.82 (m, 3H), 7.12 (m, 3H), 7.32 (t, 1H), 9.00 (s, 1H), 10.82 (s, 1H).

LRMS: m/z 503 (M−1)⁻.

mp 188-189° C.

Anal. Found: C, 58.97; H, 6.50; N, 5.49. C₂₅H₃₂N₂O₇S; 0.25 H₂O requires C, 58.98; H, 6.43; N, 5.50%.

Preparation 1

### 2-[2-(Benzyloxy)ethoxy]-6-bromopyridine

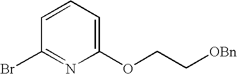

Sodium hydride (900 mg, 60% dispersion in mineral oil, 22.5 mmol) was added portionwise to an ice-cold solution of 2-(benzyloxy)ethanol (3.0 g, 20.0 mmol) in toluene (100 ml), and the solution stirred for 30 minutes. 2,6-Dibromopyridine (4.75 g, 20.0 mmol) was added, and the reaction heated under reflux for 2 hours. The cooled mixture was diluted with water (100 ml), and extracted with ethyl acetate (3×100 ml). The combined organic extracts were dried (MgSO₄), filtered and evaporated in vacuo to give the title compound as a yellow oil, (quantitative).

¹H nmr (CDCl₃, 300 MHz) δ: 3.82 (t, 2H), 4.52 (t, 2H), 4.62 (s, 2H), 6.75 (d, 1H), 7.05 (d, 1H), 7.22-7.46 (m, 6H).

### Preparation 2

### 2-Bromo-6-{[(4R)-2,2-dimethyl-1,3-dioxolan-4-yl]methoxy}pyridine

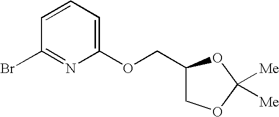

Sodium hydride (1.62 g, 60% dispersion in mineral oil, 40.5 mmol) was added portionwise to an ice-cooled solution of (R)-(−)-1,2-O-isopropylideneglycerol (4.86 g, 36.8 mmol) in toluene (100 ml), and once addition was complete, the solution was allowed to warm to room temperature and stirred for 30 minutes. 2,6-Dibromopyridine (8.72 g, 36.8 mmol) was added, and the reaction heated under reflux for 5 hours. The cooled mixture was diluted with water, the layers separated, and the aqueous phase extracted with ether. The combined organic extracts were dried (MgSO₄), filtered and evaporated in vacuo to afford the title compound as a yellow oil (quantitative).

¹H nmr (CDCl₃, 300 MHz) δ: 1.39 (s, 3H), 1.45 (s, 3H), 3.83 (dd, 1H), 4.16 (dd, 1H), 4.37 (m, 2H), 4.46 (m, 1H), 6.75 (d, 1H), 7.06 (d, 1H), 7.40 (dd, 1H).

### Preparation 3

### 2-Bromo-6-{[(4S)-2,2-dimethyl-1,3-dioxolan-4-yl]methoxy}pyridine

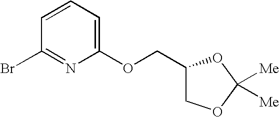

The title compound was obtained as a yellow oil (quantitative), from (S)-(−)-1,2-O-isopropylideneglycerol and 2,6-dibromopyridine, following the procedure described in preparation 2.

¹H nmr (CDCl₃, 300 MHz) δ: 1.40 (s, 3H), 1.45 (s, 3H), 3.83 (dd, 1H), 4.16 (dd, 1H), 4.37 (m, 2H), 4.48 (m, 1H), 6.76 (d, 1H), 7.06 (d, 1H), 7.41 (m/dd, 1H).

### Preparation 4

### 2-[2-(Benzyloxy)ethoxy]-6-(tributylstannyl)pyridine

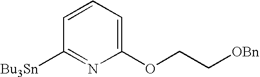

n-Butyllithium (13.8 ml, 1.6M solution in hexanes, 22.0 mmol) was added dropwise to a cooled (−78° C.) solution of the bromide from preparation 1 (20.0 mmol) in anydrous THF (100 ml), so as to maintain the internal temperature <−70° C., and the solution stirred for 20 minutes. Tri-n-butyltin chloride (6.0 ml, 22.0 mmol) was added slowly to maintain the temperature <−70° C., and the reaction then allowed to warm to room temperature over 1 hour. The reaction was diluted with water, the mixture extracted with Et₂O (2×100 ml), and the combined organic extracts dried (MgSO₄), filtered and evaporated in vacuo. The residue was purified by column chromatography on silica gel using pentane:Et₂O (98:2) as eluant, to afford the title compound as a colourless oil, (7.0 g, 67%).

¹H nmr (CDCl₃, 300 MHz) δ: 0.88 (t, 9H), 1.06 (m, 6H), 1.35 (m, 6H), 1.58 (m, 6H), 3.83 (t, 2H), 4.56 (t, 2H), 4.62 (s, 2H), 6.61 (d, 1H), 6.99 (d, 1H), 7.24-7.40 (m, 6H).

Preparation 5

### 2-{[(4R)-2,2-Dimethyl-1,3-dioxolan-4-yl]methoxy}-6-(tributylstannyl)pyridine

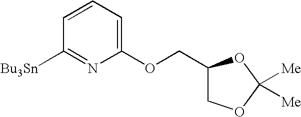

The title compound was prepared as an oil (quantitative) from the bromide of preparation 2, using a similar procedure to that described in preparation 4.

¹H nmr (CDCl₃, 300 MHz) δ: 0.88 (t, 9H), 1.06 (t, 6H), 1.25-1.40 (m, 9H), 1.45 (s, 3H), 1.50-1.70 (m, 6H), 3.83 (dd, 1H), 4.15 (dd, 1H), 4.40 (m, 2H), 4.52 (m, 1H), 6.60 (d, 1H), 7.00 (d, 1H), 7.40 (dd, 1H).

Preparation 6

2-{[(4S)-2,2-Dimethyl-1,3-dioxolan-4-yl]methoxy}-6-(tributylstannyl)pyridine

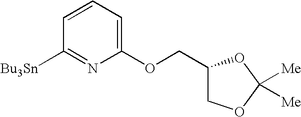

The title compound was obtained as a colourless oil (71%), from the bromide from preparation 3, following a similar procedure to that described in preparation 5.

¹H nmr (CDCl₃, 300 MHz) δ: 0.89 (t, 9H), 1.07 (t, 6H), 1.35 (m, 6H), 1.40 (s, 3H), 1.48 (s, 3H), 1.58 (m, 6H), 3.83 (dd, 1H), 4.16 (dd, 1H), 4.40 (m, 2H), 4.52 (m, 1H), 6.60 (d, 1H), 7.00 (d, 1H), 7.40 (dd, 1H).

### Preparation 7

### 3-Bromo-1-(tert-butoxy)benzene

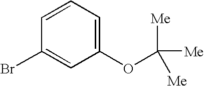

Condensed isobutylene (100 ml) was added via a dry ice/acetone cold finger, to dichloromethane (70 ml) at −30° C., followed by a solution of 3-bromophenol (21.5 g, 125 mmol) in dichloromethane (30 ml). Trifluoromethanesulphonic acid (1.5 g, 10.0 mmol) was added dropwise, the reaction cooled to −75° C., and stirred for 2 hours. Triethylamine (1.4 ml, 10.00 mmol) was then added, the solution allowed to warm to room temperature and then concentrated in vacuo to remove the isobutylene. The remaining solution was washed with water, dried (Na₂SO₄), filtered and evaporated to give the desired product as a pale yellow oil, (33 g, slightly impure).

¹H nmr (CDCl₃, 400 MHz) δ 1.37 (s, 9H), 6.89 (d, 1H), 7.04-7.20 (m, 3H).

Preparation 8

### 3-(tert-Butoxy)-phenylboronic acid

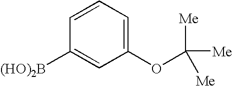

n-Butyllithium (40 ml, 2.5M in hexanes, 100 mmol) was added dropwise to a cooled (−78° C.) solution of the bromide from preparation 7 (23.9 g, 90 mmol) in tetrahydrofuran (300 ml), so as to maintain the temperature below −70° C. The resulting solution was stirred for I hour, and triisopropyl borate (30.6 ml, 135 mmol) was added dropwise over 10 minutes. The reaction was allowed to warm to room temperature, diluted with ether (150 ml) then extracted with sodium hydroxide solution (1N). The combined aqueous layers were washed with ether and then re-acidified to pH 2 using hydrochloric acid (2N). This aqueous mixture was extracted with dichloromethane (3×200 ml), the combined organic extracts dried (Na₂SO₄), filtered and concentrated in vacuo. The resulting white solid was stirred vigorously in pentane, filtered (twice) then dried under vacuum to give the title compound as a white solid, (13.1 g, 75%).

¹H nmr (CDCl₃, 400 MHz) δ 1.39 (s, 9H), 7.19 (m, 1H), 7.37 (m, 1H), 7.79 (m, 1H), 7.88 (m, 1H).

Preparation 9

### 1-Bromo-3-(2,2-diethoxyethoxy)benzene

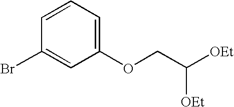

A mixture of potassium carbonate (1.5 g, 10.9 mmol), 3-bromophenol (1.73 g, 10.0 mmol) and bromoacetaldehyde diethyl acetal (1.5 ml, 9.67 mmol) in dimethylsulphoxide (10 ml) was heated at 160° C. for 1½ hours. The cooled reaction was partitioned between water (50 ml) and ethyl acetate (100 ml), and the phases separated. The aqueous layer was extracted with ethyl acetate (50 ml), the combined organic solutions washed consecutively with 1N sodium hydroxide solution, water (2×), brine and then dried (Na₂SO₄), filtered and evaporated in vacuo. The residue was purified by medium pressure column chromatography on silica gel using an elution gradient of ether:pentane (0:100 to 5:95) to afford the title compound (2.01 g, 72%).

¹H nmr (CDCl₃, 400 MHz) δ: 1.22 (t, 6H), 3.60 (m, 2H), 3.75 (m, 2H), 3.97 (d, 2H), 4.80 (t, 1H), 6.82 (d, 1H), 7.07 (m, 3H).

Preparation 10

### 3-(2,2-Diethoxyethoxy)phenylboronic acid

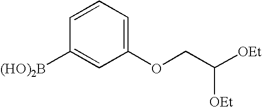

n-Butyllithium (18.5 ml, 2.5M in hexanes, 46.25 mmol) was added dropwise to a cooled (−78° C.) solution of the bromide from preparation 9 (11.4 g, 39.6 mmol) in anhydrous tetrahydrofuran (100 ml), so as to maintain the internal temperature <−70° C. This solution was stirred for 1 hour, then triisopropyl borate (1.13 g, 6.0 mmol) added slowly, and the reaction allowed to warm to room temperature over 3 hours. The mixture was cooled in an ice-bath, acidified to pH 4 using 2N hydrochloric acid, and quickly extracted with ethyl acetate (2×500 ml). The combined organic extracts were washed with water and brine, dried (Na₂SO₄), filtered and evaporated in vacuo. The residual oil was purified by medium pressure column chromatography on silica gel using an elution gradient of ether:pentane (0:100 to 50:50) to afford the title compound (8.24 g, 82%).

¹H nmr (DMSO-d₆, 400 MHz) δ: 1.14 (t, 6H), 3.58 (m, 2H), 3.66 (m, 2H), 3.94 (d, 2H), 4.80 (t, 1H), 6.98 (m, 1H), 7.22 (m, 1H), 7.37 (m, 2H), 8.00 (s, 2H).

Preparation 11

### 1-Methylsulphonyl-piperidin-4-one ethylene ketal

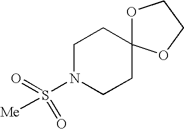

Methanesulphonyl chloride (24.8 g, 0.217 mol) was added dropwise to a solution of 4-piperidone ethylene ketal (28.2 g, 0.197 mol) and triethylamine (30.2 ml, 0.217 mol) in ether (280 ml), and the reaction stirred at room temperature for 3 hours. The mixture was washed consecutively with water (2×), hydrochloric acid (1N), and saturated sodium bicarbonate solution, dried (MgSO₄), filtered and evaporated in vacuo. The residue was triturated with hexane, filtered and dried to give the desired product as an off-white solid (41.6 g, 95%).

mp 107-109° C.

¹H nmr (CDCl₃, 400 MHz) δ: 1.78 (m, 4H), 2.75 (s, 3H), 3.32 (m, 4H), 3.92 (s, 4H).

Anal. Found: C, 43.23; H, 6.85; N, 6.23. C₈H₁₅NO₄S requires C, 43.42; H, 6.83; N, 6.33%.

Preparation 12

### 1-Isopropylsulphonyl-piperidin-4-one ethylene ketal

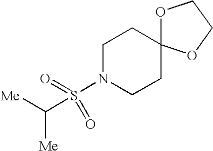

Isopropylsulphonyl chloride (5.6 ml, 50 mmol) was added dropwise to an ice-cooled solution of 4-piperidone ethylene ketal (6.4 ml, 50 mmol) and triethylamine (7.7 ml, 55 mmol) in dichloromethane (100 ml), and the reaction stirred at room temperature for 3 hours. The mixture was washed with water (2×), dried (MgSO₄), filtered and evaporated in vacuo. The residue was crystallised from ether/pentane to afford the title compound as a solid, (10.55 g, 85%).

mp 66-67° C.

¹H nmr (CDCl₃, 400 MHz) δ: 1.34 (d, 6H), 1.77 (m, 4H), 3.18 (m, 1H), 3.43 (m, 4H), 3.98 (s, 4H),

Anal. Found: C, 48.19; H, 7.74; N, 5.50. C₁₀H₁₉NO₄S requires C, 48.15; H, 7.75; N, 5.56%.

Preparation 13

### Methyl 2-(1,4-dioxa-8-azaspiro[4.5]dec-8-ylsulphonyl)acetate

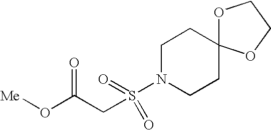

Potassium tert-butoxide (24.6 g, 219 mmol) was added portionwise to a solution of the ethylene ketal from preparation 11 (32.3 g, 146 mmol) and dimethyl carbonate (61 ml, 730 mmol) in tetrahydrofuan (200 ml), and once addition was complete, the reaction was stirred at room temperature overnight under a nitrogen atmosphere. The reaction was poured into a mixture of hydrochloric acid (1N) and ether and the layers separated. The aqueous layer was extracted with ethyl acetate, the combined organic solutions washed with brine, dried (MgSO₄), filtered and evaporated in vacuo. The residue was suspended in di-isopropyl ether, the mixture heated to reflux, cooled, and filtered, to afford the title compound as a solid, (26.7 g, 65%).

¹H nmr (CDCl₃, 400 MHz) δ: 1.77 (m, 4H), 3.42 (m, 4H), 3.78 (s, 3H), 3.92 (s, 2H), 3.95 (s, 4H),

Anal. Found: C, 42.69; H, 6.16; N, 4.93. C₁₀H₁₇NO₆S requires C, 43.00; H, 6.14; N, 5.02%.

Preparation 14

### Methyl 2-(1,4-dioxa-8-azaspiro[4.5]dec-8-ylsulphonyl)-2-methylpropanoate

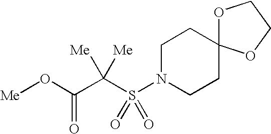

N-Butyl lithium (28 ml, 1.6M in hexanes, 44.1 mmol) was added dropwise to a cooled (−78° C.) solution of the sulphonamide from preparation 12 (10 g, 40.1 mmol) in tetrahydrofuran (100 ml), so as to maintain a temperature below −45° C. Once addition was complete the solution was allowed to warm to 0° C., and then recooled to −78° C. Methyl chloroformate (3.7 ml, 48.1 mmol) was added dropwise so as to maintain the temperature below −45° C., the reaction stirred for 30 minutes, then allowed to warm to room temperature. The reaction mixture was partitioned between ethyl acetate and water, and the layers separated. The organic phase was washed with water, dried (MgSO₄), filtered and evaporated in vacuo. The crude product was triturated with ether to give the title compound as a solid, (9.88 g, 80%).

¹H nmr (CDCl₃, 400 MHz) δ: 1.60 (s, 6H), 1.76 (m, 4H), 3.48 (m, 4H), 3.79 (s, 3H), 3.98 (s, 4H).

Anal. Found: C, 46.80; H, 6.87; N, 4.49. C₁₂H₂₁NO₆S requires C, 46.89; H, 6.89; N, 4.56%.

Preparation 15

### Methyl 4-(1,4-dioxa-8-azaspiro[4.5]dec-8-ylsulphonyl)tetrahydro-2H-pyran-4-carboxylate

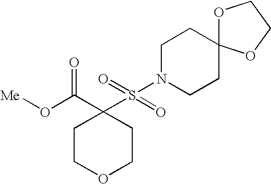

Sodium hydride (880 mg, 60% dispersion in mineral oil, 22 mmol) was added to a solution of the sulphonamide from preparation 11 (2.21 g, 10 mmol) and dimethyl carbonate (4.2 ml, 50 mmol) in dry toluene (40 ml), and the mixture heated at 90° C. for 90 minutes. Tlc analysis showed starting material present, so methanol (20 ?l) was added, and the reaction stirred at 90° C. overnight. 1-Methyl-2-pyrrolidinone (10 ml) and bis(2-bromoethyl)ether (1.63 ml, 13 mmol) were added, and the reaction stirred for a further 20 hours at 90° C., and at room temperature for 3 days. The reaction mixture was partititoned between 1N citric acid solution and ether, and the layers separated. The organic phase was washed with water, dried (MgSO₄), filtered and evaporated in vacuo. The residue was triturated with ether to give the title compound as a white solid, (1.05 g, 30%).

Alternative Method

Potassium tert-butoxide (220 ml, 1M in tetrahydrofuran, 220 mmol) was added dropwise to a solution of the acetate from preparation 13 (27.9 g, 100 mmol) and bis(2-bromoethyl)ether (16.3 ml, 130 mmol) in tetrahydrofuran (200 ml) and 1-methyl-2-pyrrolidinone (20 ml), and the reaction stirred at room temperature overnight. Tlc analysis showed starting material remaining, so tetrabutylammonium iodide (3.7 g, 10 mmol) and sodium hydride (2.0 g, 60% dispersion in mineral oil, 50 mmol) were added, and the reaction stirred for a further 72 hours. Additional 1-methyl-2-pyrrolidinone (100 ml), sodium hydride (4.0 g, 60% dispersion in mineral oil, 100 mmol) and bis(2-bromoethyl)ether (12.6 ml, 100 mmol) were added, and the reaction continued for a further 24 hours. The reaction was poured into a mixture of ether and 10% citric acid solution, and the layers separated. The aqueous phase was extracted with ether, the combined organic solutions washed with water, dried (MgSO₄), filtered and evaporated in vacuo.The residue was suspended in ether, the mixture heated to reflux, cooled and the resulting precipitate filtered, washed with ether and dried to give the title compound, (7.2 g, 21%).

¹H nmr (CDCl₃, 400 MHz) δ: 1.70 (m, 4H), 2.16 (m, 2H), 2.35 (m, 2H), 3.24 (m, 2H), 3.41 (m, 4H), 3.80 (s, 3H), 3.94 (m, 6H).

LRMS: m/z 372 (M+23)⁺

Preparation 16

### Methyl 4-(4-oxo-piperidin-1-ylsulphonyl)tetrahydro-2H-pyran-4-carboxylate

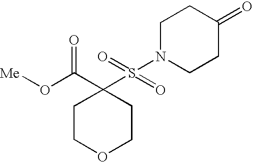

Hydrochloric acid (20 ml, 1N) was added to a solution of the ethylene ketal from preparation 15 (7.1 g, 20.3 mmol) in acetone (20 ml) and 1,4-dioxan (20 ml), and the reaction stirred at 60° C. for 6 hours, and then left at room temperature overnight. The reaction was neutralised by adding sodium bicarbonate portionwise, and this mixture concentrated in vacuo. The residue was diluted with water, then extracted with ethyl acetate (3×). The combined organic extracts were dried (MgSO₄), filtered and evaporated in vacuo.The crude product was triturated with ether/di-isopropyl ether, to give the desired product as a solid (4.1 g, 66%).

mp 158-160° C.

¹H nmr (CDCl₃, 400 MHz) δ: 2.18 (m, 2H), 2.38 (m, 2H), 2.48 (m, 4H), 3.26 (m, 2H), 3.60 (br, m, 4H), 3.82 (s, 3H), 3.98 (m, 2H).

Anal. Found: C, 47.14; H, 6.28; N, 4.54. C₁₂H₁₉NO₆S requires C, 47.20; H, 6.27; N, 4.59%.

Preparation 17

### Methyl 2-methyl-2-(4-oxo-piperidin-1-ylsulphonyl)propanoate

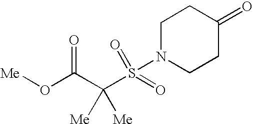

The title compound was obtained as a solid (98%) after trituration with pentane from the ethylene ketal from preparation 14, following a similar method to that described in preparation 16.

¹H nmr (CDCl₃, 400 MHz) δ: 1.67 (s, 6H), 2.57 (m, 4H), 3.68 (m, 4H), 3.80 (s, 3H).

Anal. Found: C, 45.51; H, 6.52; N, 5.14. C₁₀H₁₇NO₅S requires C, 45.61; H, 6.51; N, 5.32%.

Preparation 18

### tert-Butyl 4-[4-(4-bromo-3-methylphenyl)-4-hydroxypiperidine-1-carboxylate

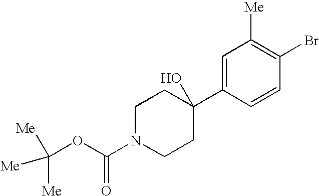

A 2.5M solution of n-butyl lithium in hexane (38 ml, 94 mmol) was added over about 10 minutes to a stirred mixture of 2-bromo-5-iodo-toluene (28 g, 94 mmol) in anhydrous ether (500 ml) under nitrogen, at about −75° C. After a further 15 minutes, a solution of t-butyl 4-oxopiperidine-1-carboxylate (17 g, 85 mmol) in anhydrous tetrahydrofuran (50 ml) was added at such a rate that the reaction temperature was maintained below −60° C.

The reaction mixture was stirred at about −75° C. for 1 hour, and allowed to warm to 0° C. and quenched with aqueous ammonium chloride solution. The organic phase was separated, washed with water, dried (MgSO₄), filtered and evaporated in vacuo. The residue was dissolved in pentane and cooled to 0° C. to crystallise the title compound, which was collected by filtration as a colourless solid (20.1 g, 64%).

m.p. 102-103° C.

¹H nmr (CDCl₃) δ: 1.48 (s, 9H), 1.51 (s, 1H), 1.70 (d, 2H), 1.96 (m, 2H), 2.40 (s, 3H), 3.22 (t, 2H), 4.02 (m, 2H), 7.15 (dd, 1H), 7.36 (d, 1H), 7.50 (d, 1H).

LRMS: m/z 369/371 (M+1)⁺

Anal. Found: C, 55.14; H, 6.58; N, 3.76. C₁₇H₂₄BrNO₃ requires C, 55.14; H, 6.53; N, 3.78%.

Preparation 19

### 4-(4-Bromo-3-methylphenyl)-1,2,3,6-tetrahydropyridine

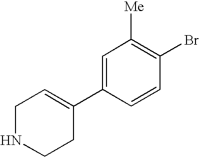

Trifluoroacetic acid (100 ml) was added to a stirred solution of the bromide from preparation 18 (20 g, 54 mmol) in dichloromethane (100 ml) at room temperature. After a further 18 hours, the reaction mixture was evaporated in vacuo and the residue basified with 2M aqueous sodium hydroxide solution to pH>12. The resulting mixture was extracted with ether, the combined extracts washed with water, dried (MgSO₄), filtered and evaporated under reduced pressure to yield the title compound as a low melting solid, (13.6 g, 100%).

¹H nmr (CDCl₃) δ: 1.60 (br, s, 1H), 2.40 (m, 5H), 3.10 (t, 2H),3.52 (m, 2H), 6.10 (br, s, 1H), 7.05 (dd, 1H), 7.22 (d, 1H), 7.46 (d, 1H).

LRMS: m/z 251/253 (M+1)⁺.

Alternative Method

Para-toluenesulphonic acid (10.27 g, 54 mmol) was added to a stirred solution of the bromide from preparation 18 (10 g, 27 mmol) in toluene (130 ml) at room temperature. The gelatinous mixture was heated to reflux in a Dean-Stark apparatus for 90 minutes, and then cooled to room temperature which resulted in a thick white precipitate. The mixture was basified with 2M sodium hydroxide solution, and extracted with ethyl acetate (3×), then the combined extracts were washed with water, dried (MgSO₄) and evaporated under reduced pressure to yield the title as a low melting solid, (6.8 g, 100%).

Preparation 20

### 4-(4-Bromo-3-methylphenyl)-1-methylsulphonyl-1,2,3,6-tetrahydropyridine

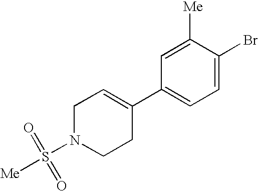

Methanesulphonyl chloride (17.5 ml, 227 mmol) was added dropwise to an ice-cooled solution of triethylamine (34.4 ml, 247 mmol) and the amine from preparation 19 (51.8 g, 206 mmol) in dichloromethane (400 ml), and the reaction then stirred at room temperature for 1 hour. Tlc analysis showed starting material remaining, so additional methanesulphonyl chloride (1.75 ml, 22.7 mmol) and triethylamine (5 ml, 35.9 mmol) were added, and stirring continued for a further hour. The reaction was diluted with hydrochloric acid (200 ml, 2N) and water (300 ml), and the phases separated. The aqueous layer was extracted with dichloromethane (2×250 ml) the combined organic extracts washed with brine (200 ml), dried (MgSO₄), filtered and concentrated in vacuo. The residual solid was triturated with isopropyl ether, filtered and dried to afford the title compound as a pale yellow solid, (65.1 g, 96%).

¹H nmr (CDCl₃, 300 MHz) δ: 2.40 (s, 3H), 2.62 (m, 2H), 2.85 (s, 3H), 3.54 (m, 2H), 3.95 (m, 2H), 6.04 (m, 1H), 7.04 (dd, 1H), 7.21 (m, 1H), 7.50 (d, 1H).

LRMS m/z 347, 349 (M+18)⁺

Preparation 21

### Methyl 2-[4-(4-bromo-3-methylphenyl)-1,2,3,6-tetrahydropyridin-1-ylsulphonyl]acetate

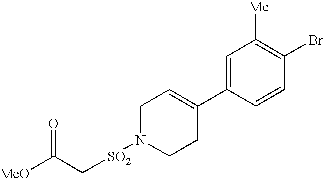

N,O-Bis(trimethylsilyl)acetamide (0.9 ml, 4.0 mmol) was added to a stirred solution of the amine from preparation 19 (2.0 g, 7.9 mmol) in anhydrous tetrahydrofuran (40 ml), under nitrogen, at room temperature. A solution of methyl chlorosulphonylacetate (1.64 g, 9.5 mmol) in anhydrous tetrahydrofuran (15 ml) was added and the reaction mixture stirred at room temperature for 18 hours. The resulting mixture was evaporated in vacuo, and partitioned between ethyl acetate and aqueous sodium bicarbonate solution. The organic layer was separated and washed with water, dried (MgSO₄), filtered and evaporated in vacuo. The residue was purified by column chromatography on silica gel, using dichloromethane as eluant, followed by crystallisation from diisopropyl ether, to give the title compound as a colourless solid, (1.65 g, 55%).

m.p. 110-112° C.

¹H nmr (CDCl₃) δ: 2.40 (s, 3H), 2.60 (m, 2H), 3.60 (t, 2H), 3.80 (s, 3H), 4.01 (s, 2H), 4.07 (m, 2H), 6.02 (br, s,1H), 7.02 (dd, 1H), 7.21 (d, 1H), 7.50 (d, 1H).

LRMS: m/z 404/406 (M+18)⁺

Anal. Found: C, 46.32; H, 4.62; N, 3.55. C₁₅H₁₈BrNO₄S requires C, 46.40; H, 4.67; N, 3.61%

Preparation 22

### Methyl 2-[4-(4-bromo-3-methylphenyl)-1,2,3,6-tetrahydropyridin-1-ylsulphonyl]-2-methyl-propanoate

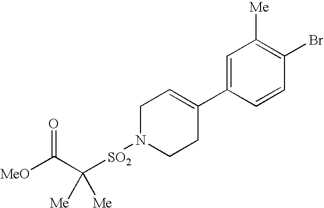

Iodomethane (2 ml, 32.1 mmol) was added to a stirred mixture of the acetate from preparation 21 (5 g, 12.9 mmol) and potassium carbonate (5.4 g, 39.1 mmol), in anhydrous dimethylsulfoxide (50 ml), under nitrogen, at room temperature. After 24 hours the reaction mixture was partitioned between ether and water, separated, and the organic layer was washed with water, dried (MgSO₄), filtered and evaporated in vacuo. The residue was purified by flash chromatography, using diethyl ether:pentane (40:60 to 100:0) as eluant, followed by crystallisation from diisopropyl ether, to give the title compound as a colourless solid, (4.7 g, 87%).

m.p. 100-101° C.

¹H nmr (CDCl₃) δ: 1.67 (s, 6H), 2.40 (s, 3H), 2.58 (m, 2H), 3.60 (t, 2H), 3.80 (s, 3H), 4.08 (m, 2H), 6.00 (br, s, 1H), 7.03 (dd, 1H), 7.21 (d, 1H), 7.49 (d, 1H).

Anal. Found: C, 49.00; H, 5.33; N, 3.28. C₁₇H₂₂BrNO₄S requires C, 49.04; H, 5.33; N, 3.36%.

Preparation 23

### Methyl 4-[4-(4-bromo-3-methylphenyl)-1,2,3,6-tetrahydropyridin-1-ylsulphonyl]tetrahydro-2H-pyran-4-carboxylate

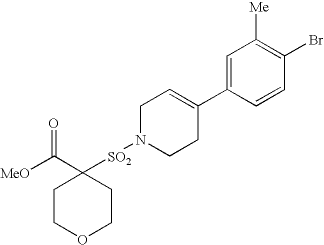

Bis-2-iodoethyl ether (3.9 g, 12.0 mmol) was added to a stirred mixture of the acetate from preparation 21 (3.6 g, 9.3 mmol) and potassium carbonate (3.8 g, 27.8 mmol), in anhydrous dimethylsulfoxide (50 ml), under nitrogen, at room temperature. After 18 hours the reaction mixture was partitioned between diethyl ether and water, separated, and the organic layer was washed with water, dried (MgSO₄), filtered and evaporated in vacuo. The residue was purified by flash chromatography, using a mixture of dichloromethane and methanol (99:1) as eluant, followed by crystallisation from diisopropyl ether, to give the title compound as a colourless solid, (3.43 g, 80%).

m.p. 128-130° C.

¹H nmr (CDCl₃) δ: 2.23 (m, 2H), 2.40 (s, 3H), 2.42 (m, 2H), 2.58 (m, 2H), 3.30 (m, 2H), 3.58 (m, 2H), 3.87 (s, 3H), 4.00-4.10 (m, 4H), 6.00 (br, s, 1H), 7.02 (dd, 1H), 7.21 (d, 1H), 7.49 (d, 1H),

LRMS: m/z 477 (M+18)⁺

Anal. Found: C, 49.92; H, 5.40; N, 2.90. C₁₉H₂₄BrNO₅S requires C, 49.78; H, 5.28; N, 3.06%.

Preparation 24

### 4-(4-Bromo-3-methylphenyl)-1-(methylsulphonyl)piperidine

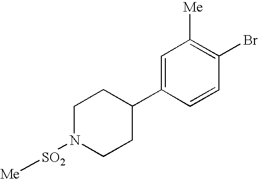

Triethylsilane (47.2 ml, 296 mmol), followed by trifluoromethanesulphonic acid (1.73 ml, 19.7 mmol) were added to a solution of the sulphonamide from preparation 20 (65.0 g, 197 mmol) in dichloromethane (300 ml) and trifluoroacetic acid (300 ml), and the reaction stirred at room temperature for an hour. Tlc analysis showed starting material remaining, so additional triethylsilane (75.2 ml, 471 mmol) and trifluoromethanesulphonic acid (0.86 ml, 9.8mmol) were added and the reaction stirred for a further 20 hours at room temperature. The reaction was concentrated in vacuo, the residue poured into saturated aqueous potassium carbonate solution, and the mixture extracted with dichloromethane (3×650 ml). The combined organic extracts were washed with brine (500 ml), dried (MgSO₄), filtered and concentrated in vacuo. The crude product was triturated with hot methanol/hexane, filtered and dried to give the title compound (52.43 g, 80%) as a buff-coloured solid.

¹H nmr (CDCl₃, 400 MHz) δ: 1.78 (m, 2H), 1.90 (m, 2H), 2.37 (s, 3H), 2.52 (m, 1H), 2.77 (m, 5H), 3.94 (m, 2H), 6.83 (m, 1H), 7.02 (s, 1H), 7.42 (m, 1H).

LRMS: m/z 354, 356 (M+23)⁺

Preparation 25

### Methyl 2-[4-(4-bromo-3-methylphenyl)piperidin-1-ylsulphonyl]acetate

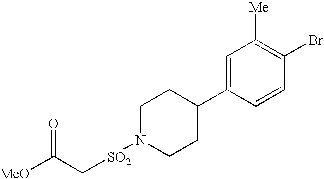

Sodium hydride (12.2 g, 60% dispersion in mineral oil, 305 mmol) was added to a solution of the sulphonamide from preparation 24 (50.61 g, 152 mmol) and dimethylcarbonate (63.8 ml, 760 mmol) in toluene (600 ml), and the reaction heated under reflux for 1½ hours. The reaction was partitioned between ethyl acetate (1000 ml), and cooled hydrochloric acid (600 ml, 1N), and the layers separated. The aqueous layer was extracted with ethyl acetate (500 ml), the combined organic extracts washed with brine (3×300 ml), dried (MgSO₄), filtered and concentrated in vacuo. The residue was triturated with hexane, and the solid filtered. This was re-crystallised from di-isopropyl ether and dried in vacuo to give the title compound as buff-coloured crystals, (40.9 g, 69%).

¹H nmr (CDCl₃, 400 MHz) δ: 1.77 (m, 2H), 1.84 (m, 2H), 2.37 (s, 3H), 2.58 (m, 1H), 2.97 (m, 2H), 3.80 (s, 3H), 3.96 (m, 4H), 6.84 (m, 1H), 7.02 (s, 1H), 7.42 (d, 1H).

LRMS m/z 412, 414 (M+23)⁺

Preparation 26

### Methyl 2-[4-(4-bromo-3-methylphenyl)piperidin-1-ylsulphonyl]-2-methyl-propanoate

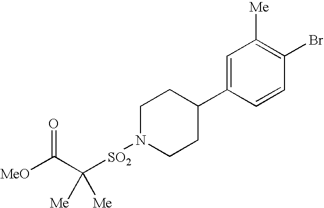

Triethylsilane (1.43 ml, 9.0 mmol) followed by trifluoromethanesulphonic acid (0.02 ml, 0.3 mmol) were added to a solution of the 1,2,3,6-tetrahydropyridine from preparation 22 (1.25 g, 3.0 mmol) and trifluoroacetic acid (15 ml) in dichloromethane (15 ml), and the reaction was stirred for an hour at room temperature. The reaction mixture was concentrated in vacuo, the residue diluted with dichloromethane (25 ml), then partitioned between ethyl acetate (150 ml) and saturated sodium bicarbonate solution (150 ml), and the layers separated. The aqueous phase was extracted with ethyl acetate (2×35 ml), the combined organic solutions dried (MgSO₄), filtered and evaporated in vacuo. The residual solid was triturated with di-isopropyl ether to give the title compound as a white solid, (963 mg, 77%).

mp 103-106° C.

¹H nmr (DMSO-d₆, 400 MHz) δ: 1.52 (m, 8H), 1.77 (m, 2H), 2.28 (s, 3H), 2.63 (m, 1H), 3.0 (m, 2H), 3.70 (m, 5H), 6.98 (dd, 1H), 7.20 (s, 1H), 7.42 (dd, 1H).

Anal. Found: C, 48.42; H, 5.74; N, 3.27. C₁₇H₂₄BrNSO₄ requires C, 48.81; H, 5.78 N%, 3.35%.

Preparation 27

### Methyl 4-[4-(4-bromo-3-methylphenyl)piperidin-1-ylsulphonyl]tetrahydro-2H-pyran-4-carboxylate

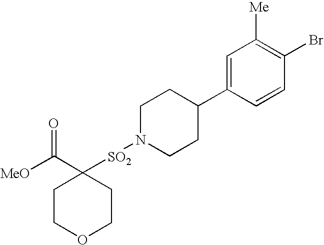

Sodium hydride (60% dispersion in mineral oil, 1.16 g, 29.0 mmol) was added to a stirred solution of the acetate from preparation 25 (10.14 g, 26.0 mmol) in N-methyl pyrrolidinone (60 ml) at ambient temperature under nitrogen. After 45 minutes, bis-2-bromoethyl ether (4.26 ml, 33.8 mmol) was added to the stirred mixture, and after a further 150 minutes an additional portion of sodium hydride (60% dispersion in mineral oil; 1.16 g, 29 mmol) was added, and the mixture left stirring for 18 hours. The solvent was removed under reduced pressure, and the residues was partitioned between ethyl acetate and water. The organic layer was collected, washed with brine, dried (MgSO₄), and evaporated under reduced pressure. The residue was crystallised from ethyl acetate and diisopropyl ether to give the title compound as a colourless solid (7.34 g, 61%). The filtrate was evaporated and purified by flash chromatography eluting with dichloromethane, and crystallisation from ethyl acetate and diisopropyl ether to give an additional batch of the title compound as a colourless solid (1.86 g, 15%). A small sample was recrystallised from ethyl acetate for further characterisation.

m.p. 162-163° C.

¹Hnmr (CDCl₃) δ: 1.65-1.83 (m, 4H), 2.20 (m, 2H), 2.38 (s, 3H), 2.40 (m, 2H), 2.57 (m, 1H), 3.00 (m, 2H), 3.29 (m, 2H), 3.85 (s, 3H), 3.87-4.00 (m, 4H), 6.83 (d, 1H), 7.02 (s, 1H), 7.41 (d, 1H).

LRMS: m/z 460/462 (M+1)⁺.

Anal. Found: C,49.49; H,5.68; N,2.93. C₁₉H₂₆BrNO₅S requires C,49.57; H,5.69; N,3.04%.

Alternative Route: Triethylsilane (50 ml, 0.30 mol) was added dropwise over 2 min to a solution of the carbinol from preparation 130 (60 g, 0.12 mol) in dichloromethane (150 ml) and trifluoroacetic acid (150 ml), at 0° C., under nitrogen. Triflic acid (0.53 ml, 6.0 mmol) was added dropwise over 10 min and the resulting mixture was stirred at 0° C. for 4 h. Dichloromethane (300 ml) and demineralised water (300 ml) were added and the aqueous phase was separated. The organic phase was washed with water (200 ml), saturated sodium bicarbonate solution (2×200 ml) and demineralised water (200 ml) and then concentrated in vacuo to a colourless solid. The solid was slurried in hot ethyl acetate (300 ml) for 20 min and the mixture was cooled to 0° C. and then filtered. The residue was dried in vacuo to leave the title compound as a colourless solid (53 g, 92%).

Preparation 28

### Methyl 1-benzyl-4-[4-(4-bromo-3-methylphenyl)piperidin-1-ylsulphonyl]-4-piperidinecarboxylate

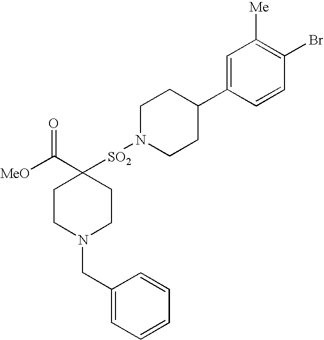

The acetate from preparation 25 (4.17 g, 10.7 mmol) was added portionwise to a suspension of sodium hydride (994 mg, 60% dispersion in mineral oil, 33.1 mmol) in 1-methyl-2-pyrrolidinone (40 ml), and the resulting solution stirred for an hour. Tetra-butyl ammonium bromide (3.44 g, 10.7 mmol) and N-benzyl-bis-(2-chloroethyl)amine (2.73 g, 10.1 mmol) were added portionwise, and once addition was complete, the reaction was stirred at 60° C. for 6 hours. The cooled reaction was partitioned between water and ethyl acetate, the layers separated, and the aqueous phase extracted with ethyl acetate. The combined organic extracts were washed with water, dried (Na₂SO₄), filtered and concentrated in vacuo. The crude product was purified by column chromatography on silica get twice, using an elution gradient of dichloromethane:ether (100:0 to 90:10) to afford the title compound (3.04 g, 52%).

¹H nmr (CDCl₃, 400 MHz) δ: 1.63-1.81 (m, 4H), 1.88 (m, 2H), 2.16 (m, 2H), 2.36 (s, 3H), 2.42 (m, 2H), 2.55 (m, 1H), 2.88 (m, 2H), 2.98 (m, 2H), 3.40 (s, 2H), 3.82 (m, 5H), 6.83 (d, 1H), 7.00 (s, 1H), 7.22 (m, 5H), 7.40 (d, 1H).

LRMS m/z 549, 551 (M+1)⁺

Preparation 29

### Methyl 2-methyl-2-{4-(trifluoromethanesulphonyloxy]-1,2,3,6-tetrahydropyridin-1-ylsulphonyl}propanoate

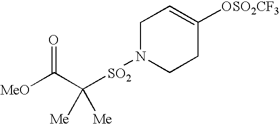

2,6-Di-tert-butyl-4-methylpyridine (3.7 g, 18 mmol) was added to a solution of the ketone from preparation 17 (3.8 g, 14.5 mmol) in dichloromethane (50 ml), and the solution then cooled to 4° C. Trifluoromethane sulphonic anhydride (2.95 ml, 17.5 mmol) was added dropwise, and the reaction then stirred at room temperature for 17 hours. Tlc analysis showed starting material remaining, so additional 2,6-di-tert-butyl-4-methylpyridine (3.7 g, 18 mmol) and trifluoromethane sulphonic anhydride (2.7 ml, 16 mmol) were added portionwise to the stirred reaction over the following 4 days. The mixture was then filtered, the filtrate concentrated in vacuo, and the residue triturated with ether. The resulting solid was filtered off, and the filtrate evaporated in vacuo. This crude product was purified by column chromatography on silica gel using an elution gradient of hexane:ethyl acetate (91:9 to 50:50) to afford the title compound (4.25 g, 74%) as a white solid.

¹H nmr (CDCl₃, 400 MHz) δ: 1.64 (s, 6H), 2.56 (m, 2H), 3.60 (m, 2H), 3.79 (s, 3H), 4.06 (m, 2H), 5.80 (m, 1H).

Anal. Found: C, 33.62; H, 4.03; N, 3.43. C₁₁H₁₆F₃NO₇S₂ requires C, 33.42; H, 4.08; N, 3.54%.

Preparation 30

### Methyl 2-[4-(4-{3-formylphenyl}-3-methylphenyl)-piperidin-1-ylsulphonyl]tetrahydro-2H-pyran-4-carboxylate

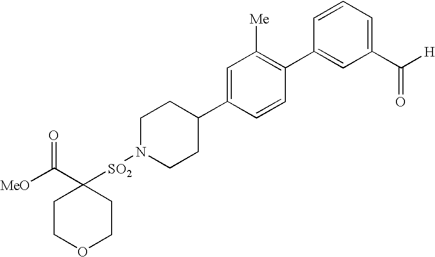

A mixture of the bromide from preparation 27 (4.02 g, 8.73 mmol), 3-formylphenylboronic acid (1.83 g, 11.56 mmol), cesium fluoride (3.46 g, 22.8 mmol), tris(dibenzylideneacetone)palladium (0) (430 mg, 0.47 mmol) and tri(o-tolyl)phosphine (284 mg, 0.93 mmol) in 1,2-dimethoxyethane (70 ml) was heated under reflux for 6 hours. The cooled reaction was diluted with water and the mixture extracted with ethyl acetate (3×). The combined organic extracts were washed with brine, dried (MgSO₄), filtered and concentrated under reduced pressure. The residue was purified by column chromatography on silica gel using an elution gradient of ethyl acetate:hexane (25:75 to 40:60), and triturated with di-isopropyl ether to give the title compound as a solid, (2.69 g, 63%).

¹H nmr (CDCl₃, 400 MHz) δ: 1.75-1.95 (m, 4H), 2.20 (m, 5H), 2.40 (m, 2H), 2.62 (m, 1H), 3.03 (m, 2H), 3.30 (m, 2H), 3.82-4.02 (m, 7H), 7.07 (m, 2H), 7.16 (m, 1H), 7.56 (m, 2H), 7.81 (m, 2H), 10.02 (s, 1H).

LRMS: m/z 508 (M+23)⁺

Preparation 31

### Methyl 2-[4-(4-{6-[2-benzyloxy]ethoxypyridin-2-yl}-3-methylphenyl)-1,2,3,6-tetrahydropyridin-1-ylsulphonyl]-2-methyl-propanoate

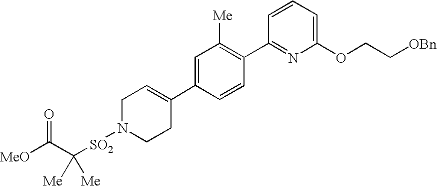

A mixture of the stannane from preparation 4 (2.8 g, 5.4 mmol) and the bromide from preparation 22 (1.5 g, 3.62 mmol), and tetrakis(triphenylphosphine)palladium (0) (205 mg, 0.18 mmol) in toluene (35 ml) was heated under reflux overnight. The cooled mixture was evaporated in vacuo and the residue purified by column chromatography on silica gel using pentane:ethyl acetate (75:25) as eluant, to afford the title compound as a colourless oil, (1.7 g, 83%).

¹H nmr (CDCl₃, 300 MHz) δ: 1.69 (s, 6H), 2.42 (s, 3H), 2.64 (m, 2H), 3.62 (t, 2H), 3.82 (m, 5H), 4.14 (m, 2H), 4.56 (t, 2H), 4.62 (s, 2H), 6.06 (s, 1H), 6.77 (d, 1H), 7.0 (d, 1H), 7.22-7.42 (m, 8H), 7.62 (m, 1H).

LRMS: m/z 565 (M+1)⁺

Preparation 32

### Methyl 4-[4-(4-{6-[2-benzyloxy]ethoxypyridin-2-yl}-3-methylphenyl)-1,2,3,6-tetrahydropyridin-1-ylsulphonyl]tetrahydro-2H-pyran-4-carboxylate

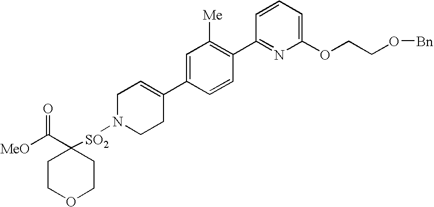

A mixture of the stannane from preparation 4 (1.74 g, 3.36 mmol) and the bromide from preparation 23 (1.1 g, 2.4 mmol) and tetrakis(triphenylphosphine)palladium (0) (138 mg, 0.14 mmol) in toluene (16 ml) was heated under reflux for 4 hours. The cooled reaction was diluted with water, and the mixture extracted with ether (3×). The combined organic extracts were washed with brine, dried (MgSO₄), filtered through Arbocel® and evaporated in vacuo. The residual yellow oil was purified by column chromatography on silica gel using an elution gradient of pentane:ether (50:50 to 25:75) to afford the title compound as a pale yellow oil, (1.18 g, 81%).

¹H nmr (CDCl₃, 400 MHz) δ: 2.22 (m, 2H), 2.42 (m, 5H), 2.62 (m, 2H), 3.34 (m, 2H), 3.60 (m, 2H), 3.82 (t, 2H), 3.88 (s, 3H), 4.01 (m, 2H), 4.09 (m, 2H), 4.55 (t, 2H), 4.61 (s, 2H), 6.05 (m, 1H), 6.76 (d, 1H), 6.99 (d, 1H), 7.21-7.41 (m, 78H), 7.61 (m, 1H).

LRMS: m/z 607 (M+1)⁺

Preparation 33

### Methyl 1-benzyl-4-{[4-(4-{6-[2-benzyloxyethoxy)pyridin-2-yl}-3-methylphenyl)piperidin-1--yl]sulphonyl}-piperidin-4-carboxylate

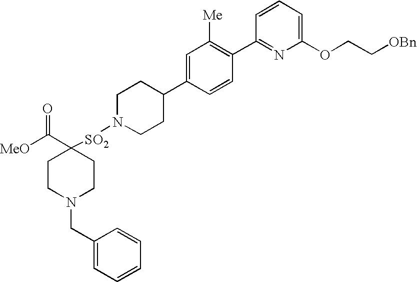

The stannane from preparation 4 (4.05 g, 7.8 mmol), followed by tris(triphenylphosphine)palladium (0) (410 mg, 0.35 mmol) were added to a solution of the bromide from preparation 28 (3.91 g, 7.1 mmol) in toluene (50 ml), and the reaction de-gassed, then heated under a nitrogen atmosphere reflux for 7 hours. Aqueous potassium fluoride solution (20 ml, 25%) was added to the cooled reaction, the mixture stirred at room temperature for 20 minutes, then filtered through Arbocel®. The filtrate was diluted with ethyl acetate, washed with brine, dried (Na₂SO₄), filtered and evaporated in vacuo. The residue was purified by column chromatography on silica gel twice, using an elution gradient of ethyl acetate:hexane (40:60 to 60:40) to give the desired product as a yellow crystalline solid, (2.77 g, 56%).

¹H nmr (CDCl₃, 400 MHz) δ: 1.74-1.95 (m, 6H), 2.17 (m, 2H), 2.37 (s, 3H), 2.44 (m, 2H), 2.60 (m, 1H), 2.88 (m, 2H), 3.00 (m, 2H), 3.40 (s, 2H), 3.80 (m, 5H), 3.88 (m, 2H), 4.52 (t, 2H), 4.59 (s, 2H), 6.70 (d, 1H), 6.95 (d, 1H), 7.03 (m, 2H), 7.18-7.37 (m, 11H), 7.58 (m, 1H).

LRMS: m/z 699 (M+1)⁺

Preparation 34

### Methyl 2-[4-(4-{3-[2,2-diethoxyethoxy]phenyl}-3-methylphenyl)-1,2,3,6-tetrahydropyridin-1-ylsulphonyl]-2-methyl-propanoate

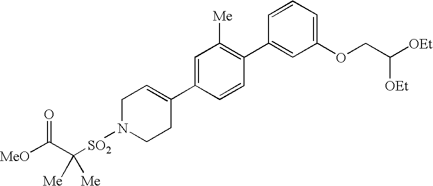

A mixture of cesium fluoride (1.81 g, 11.92 mmol), tri-o-tolyl phosphine (180 mg, 0.59 mmol), tris(dibenzylideneacetone)dipalladium (0) (280 mg, 0.31 mmol) and the boronic acid from preparation 10 (1.83 g, 7.2 mmol) and the bromide from preparation 22 (2.5 g, 6.0 mmol) in anhydrous 1,2-dimethoxyethane (60 ml), was heated under reflux for 5½ h. The cooled reaction mixture was partitioned between water and ethyl acetate, and this mixture filtered through Arbocel®. The filtrate was separated, the organic phase washed with water, then brine, dried (Na₂SO₄), filtered and evaporated in vacuo. The residual green oil was purified by medium pressure column chromatography on silica gel using an elution gradient of pentane:ethyl acetate (100:0 to 85:15) to afford the title compound, (3.04 g, 93%).

¹H nmr (CDCl₃, 300 MHz) δ: 1.24 (t, 6H), 1.69 (s, 6H), 2.28 (s, 3H), 2.64 (m, 2H), 3.62 (m, 4H), 3.80 (m, 5H), 4.04 (d, 2H), 4.12 (m, 2H), 4.84 (t, 1H), 6.06 (m, 1H), 6.92 (m, 3H), 7.14-7.38 (m, 4H).

LRMS: m/z 563 (M+18)⁺

Preparation 35

### Methyl 2-[(4-{4-[6-(2-hydroxyethoxy)pyridin-2-yl]-3-methylphenyl}-piperidin-1-yl)sulphonyl]-2-methyl-propanoate

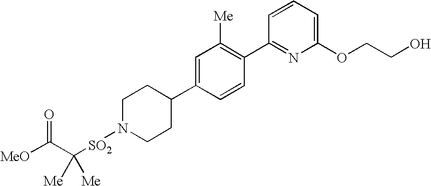

A mixture of the benzyl ether from preparation 31 (1.7 g, 3.0 mmol), ammonium formate (3.0 g, 50.0 mmol), palladium hydroxide on carbon (500 mg) and acetic acid (10 ml) in methanol (30 ml) was heated under reflux overnight. Additional ammonium formate (1.5 g, 25.0 mmol) and palladium hydroxide on carbon (1.5 g) were added and the reaction heated under reflux for a further 72 hours. The cooled mixture was filtered through Arbocel®, and the filter pad washed well with ethyl acetate. The combined filtrates were neutralised using saturated sodium bicarbonate solution, the phases separated, and the aqueous layer extracted with ethyl acetate (2×100 ml). The combined organic extracts were dried (MgSO₄), filtered and evaporated in vacuo to give the title compound as a colourless solid, (1.2 g, 84%).

mp 108-111° C.

¹H nmr (CDCl₃, 300 MHz) δ: 1.64 (s, 6H), 1.78-1.94 (m, 4H), 2.40 (s, 3H), 2.65 (m, 1H), 3.07 (m, 2H), 3.82 (s, 3H), 3.97 (m, 4H), 4.50 (t, 2H), 6.7 (d, 1H), 7.00 (d, 1H), 7.10 (m, 2H), 7.38 (d, 1H), 7.65 (m, 1H).

LRMS: m/z 477 (M+1)⁺

Preparation 36

### Methyl 4-{[4-(4-{6-[2-hydroxyethoxy]pyridin-2-yl}-3-methylphenyl)piperidin-1-yl]sulphonyl)tetrahydro-2H-pyran-4-carboxylate

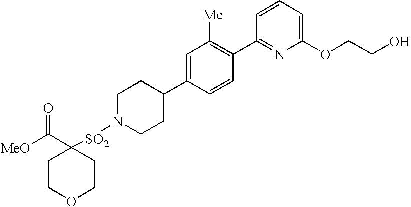

The title compound was prepared from the benzyl ether from preparation 32 in 93% yield, following a similar procedure to that described in preparation 35.

¹H nmr (CDCl₃, 300 MHz) δ: 1.70-1.95 (m, 4H), 2.22 (m, 2H), 2.40 (m, 5H), 2.64 (m, 1H), 3.06 (m, 2H), 3.34 (m, 2H), 3.92 (m, 7H), 4.00 (m, 2H), 4.50 (t, 2H), 6.78 (d, 1H), 7.00 (d, 1H), 7.10 (m, 2H), 7.38 (d, 1H), 7.65 (m, 1H).

LRMS: m/z 519 (M+1)⁺

Preparation 37

### Methyl 4-({4-[4-(6-{[(4R)-2,2-dimethyl-1,3-dioxolan-4-yl]methoxypyridin-2-yl)-3-methylphenyl]piperidin-1-yl}sulphonyl)tetrahydro-2H-pyran-4-carboxylate

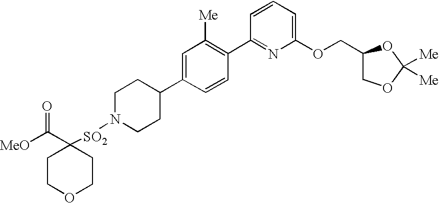

A mixture of the stannane from preparation 5 (2.0 g, 4.97 mmol) and the bromide from preparation 27 (1.76 g, 3.82 mmol) and tetrakis(triphenylphosphine)palladium (0) (242 mg, 0.21 mmol) in toluene (50 ml) was heated under reflux for 7 hours. The cooled mixture was concentrated under reduced pressure and the residue purified by column chromatography on silica gel twice, using an elution gradient of ether:pentane (66:34 to 34:66) to give the title compound as a white solid, (1.29 g, 57%).

¹H nmr (CDCl₃, 300 MHz) δ: 1.40 (s, 3H), 1.46 (s, 3H), 1.77-1.95 (m, 4H), 2.21 (m, 2H), 2.40 (m, 5H), 2.64 (m, 1H), 3.04 (m, 2H), 3.34 (m, 2H), 3.81-4.04 (m, 8H), 4.15 (dd, 1H), 4.40 (m, 2H), 4.50 (m, 1H), 6.75 (d, 1H), 7.00 (d, 1H), 7.09 (m, 2H), 7.38 (d, 1H), 7.62 (m, 1H).

LRMS: m/z 611 (M+23)⁺

Preparation 38

### Methyl 4-({4-[4-(6-{[(4S)-2,2-dimethyl-1,3-dioxolan-4-yl]methoxy}pyridin-2-yl)-3-methylphenyl]piperidin-1-yl}sulphonyl)tetrahydro-2H-pyran-4-carboxylate

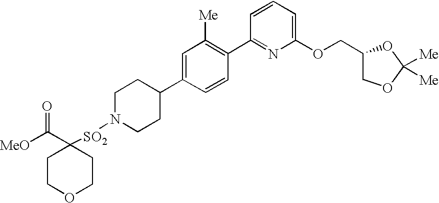

The title compound was obtained as a white solid (65%), after recrystallisation from methanol, from the stannane from preparation 6 and the bromide from preparation 27, following a similar procedure to that described in preparation 37.

¹H nmr (CDCl₃, 300 MHz) δ: 1.40 (s, 3H), 1.46 (s, 3H), 1.78-1.95 (m, 4H), 2.21 (m, 2H), 2.42 (m, 5H), 2.65 (m, 1H), 3.08 (m, 2H), 3.35 (m, 2H), 3.81-4.05 (m, 8H), 4.14 (dd, 1H), 4.40 (m, 2H), 4.50 (m, 1H), 6.76 (d, 1H), 6.99 (d, 1H), 7.08 (m, 2H), 7.38 (d, 1H), 7.62 (m, 1H).

LRMS: m/z 589 (M+1)⁺

Preparation 39

### Methyl 4-{[4-(4-{6-[(2S)-2,3-dihydroxy-1-propoxy]pyridin-2-yl}-3-methylphenyl)piperidin-1-yl]sulphonyl}tetrahydro-2H-pyran-4-carboxylate

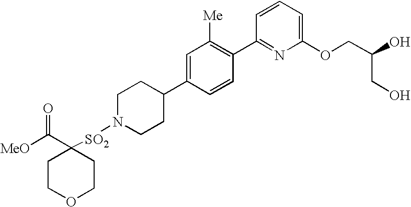

A solution of the dioxolane from preparation 37 (799 mg, 1.36 mmol) in 1,4-dioxan (10 ml) was added to an ice-cooled solution of hydrochloric acid (30 ml, 2N), and the reaction stirred for 75 minutes. The solution was poured into saturated sodium bicarbonate solution (200 ml), and the resulting precipitate filtered and dried. The solid was recrystallised from ethy acetate/di-isopropyl ether, to afford the desired product as a white powder, (642 mg, 86%).

¹H nmr (CDCl₃, 300 MHz) δ: 1.70-2.42 (m, 12H), 2.64 (m, 1H), 3.04 (m, 2H), 3.34 (m, 2H), 3.63 (m, 6H), 3.84-4.19 (m, 5H), 4.50 (m, 2H), 6.77 (d, 1H), 7.00 (d, 1H), 7.09 (m, 2H), 7.09 (m, 2H), 7.35 (d, 1H), 7.68 (m, 1H).

Preparation 40

### Methyl 4-{[4-(-4-{6-[(2R)-2,3-dihydroxy-1-propoxy]pyridin-2-yl}-3-methylphenyl)piperidin-1-yl]sulphonyl}tetrahydro-2H-pyran-4-carboxylate

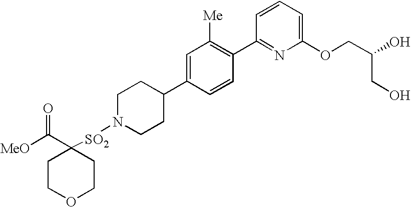

The title compound was obtained as a white crystalline solid (86%), from the dioxolane from preparation 38, following the procedure described in preparation 39.

¹H nmr (CDCl₃, 400 MHz) δ: 1.76-1.92 (m, 4H), 2.21 (m, 2H), 2.40 (m, 5H), 2.50 (t, 1H), 2.64 (m, 1H), 3.06 (m, 2H), 3.34 (m, 2H), 3.64 (m, 2H), 3.72 (m, 5H), 4.00 (m, 3H), 4.12 (d, 1H), 4.50 (m, 2H), 6.78 (d, 1H), 7.01 (d, 1H), 7.10 (m, 2H), 7.36 (d, 1H), 7.68 (m, 1H).

LRMS: m/z 571 (M+23)⁺

Preparation 41

### Methyl 4-{[4-(4-{6-[2-hydroxyethoxy]pyridin-2-yl}-3-methylphenyl)piperidin-1-yl]sulphonyl}-piperidine-4-carboxylate

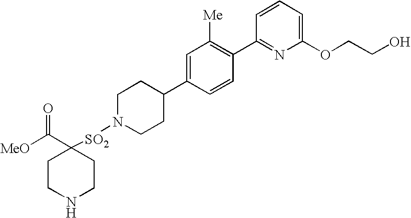

A mixture of the benzyl piperidine from preparation 33 (3.32 g, 4.76 mmol), ammonium formate (3.0 g, 47.6 mmol) and palladium hydroxide on carbon (3.32 g) in a solution of acetic acid:methanol:tetrahydrofuran (2:2:1, 30 ml) was heated under reflux for 2 hours. The cooled reaction was filtered through Arbocel®, washing through with tetrahydrofuran, and the filtrate concentrated in vacuo. The residue was partitoned between water and ethyl acetate, and the layers separated. The organic phase was dried (Na₂SO₄), filtered and evaporated in vacuo. The crude product was purified by column chromatography on silica gel using an elution gradient of dichloromethane:methanol (90:10 to 85:15) to afford the title compound, (1.28 g, 52%).

¹H nmr (CDCl₃, 400 MHz) δ: 1.73-1.88 (m, 4H), 2.00 (m, 2H), 2.38 (s, 3H), 2.42-2.64 (m, 5H), 3.02 (m, 2H), 3.16 (m, 2H), 3.85 (m, 7H), 4.46 (t, 2H), 6.73 (d, 1H), 6.98 (d, 1H), 7.05 (m, 2H), 7.34 (d, 1H), 7.60 (m, 1H).

LRMS: m/z 518 (M+1)⁺

Preparation 42

### Methyl 4-{[4-(4-{6-[2-hydroxyethoxy]pyridin-2-yl}-3-methylphenyl)piperidin-1-methylpiperidine-4-carboxylate

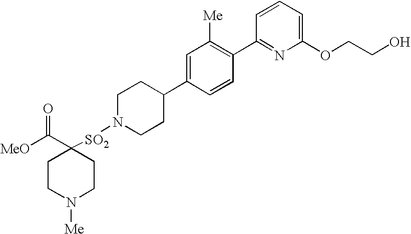

Formaldehyde (0.49 ml, 37 wt. % in water, 4.9 mmol) was added to a solution of the piperidine from preparation 41 (634 mg, 1.22 mmol) in dichloromethane (30 ml), and the solution was stirred vigorously at room temperature for 30 minutes. Sodium triacetoxyborohydride (519 mg, 2.45 mmol) was added and the reaction was stirred at room temperature for 20 hours. The reaction was washed with water, dried (Na₂SO₄), filtered and evaporated in vacuo. The crude product was purified by column chromatography on silica gel using dichloromethane:methanol (95:5) as eluant to give the title compound (559 mg, 86%).

¹H nmr (CDCl₃, 400 MHz) δ: 1.76-1.95 (m, 6H), 2.20 (m, 5H), 2.38 (s, 3H), 2.50 (m, 2H), 2.62 (m, 1H), 2.90 (m, 2H), 3.03 (m, 2H), 3.84 (s, 3H), 3.94 (m, 4H), 4.48 (m, 2H), 6.76 (d, 1H), 6.99 (d, 1H), 7.06 (m, 2H), 7.35 (d, 1H), 7.63 (m, 1H).

LRMS: m/z 554 (M+23)⁺

Preparation 43

### Methyl 1-(tert-butoxycarbonyl)-4-{[4-(4-{6-[2-hydroxyethoxy]pyridin-2-yl}-3-methylphenyl)piperidin-1-yl]sulphonyl}-4-piperidinecarboxylate

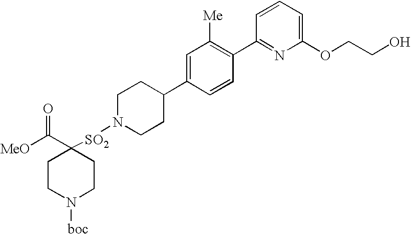

Triethylamine (175 μl, 1.26 mmol) was added to a solution of the amine from preparation 41 (594 mg, 1.15 mmol) in dichloromethane (100 ml), followed by portionwise addition of di-tert-butyl dicarbonate (262 mg, 1.20 mmol). The reaction mixture was stirred at room temperature for an hour, then concentrated in vacuo to a volume of 20 ml. The solution was diluted with ether (150 ml), washed with hydrochloric acid (0.5N), brine, then dried (MgSO₄), filtered and evaporated in vacuo. The residue was purified by column chromatography on silica gel using dichloromethane:methanol (95:5) as eluant to give the title compound (653 mg, 92%) as a white foam.

¹H nmr (CDCl₃, 400 MHz) δ: 1.42 (s, 9H), 1.75-1.90 (m, 4H), 2.01 (m, 2H), 2.38 (s, 3H), 2.45 (m, 2H), 2.63 (m, 3H), 3.02 (m, 2H), 3.50 (m, 1H), 3.87 (m, 7H), 4.17 (m, 2H), 4.46 (m, 2H), 6.75 (m, 1H), 6.98 (m, 1H), 7.05 (m, 2B), 7.35 (m, 1H), 7.62 (m, 1H).

LRMS: m/z 640 (M+23)⁺

Preparation 44

### Methyl 2-[4-(4-{3-tert-butoxyphenyl}-3-methylphenyl)-piperidin-1-ylsulphonyl]acetate

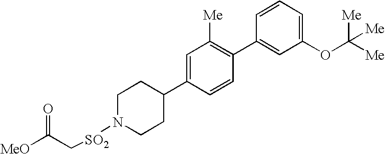

Nitrogen was bubbled through a mixture of cesium fluoride (3.71 g, 24.44 mmol), tri-o-tolyl phosphine (34 mg, 0.11 mmol), tris(dibenzylideneacetone)dipalladium (0) (50 mg, 0.05 mmol) the bromide from preparation 25 (4.27 g, 11.0 mmol) and the boronic acid from preparation 8 (3.2 g, 16.5 mmol) in anhydrous 1,2-dimethoxyethane (40 ml). The reaction was then heated at 90° C. under a nitrogen atmosphere for 50 hours. The cooled reaction mixture was diluted with ethyl acetate, the mixture washed with water (3×), dried (MgSO₄), filtered and concentrated in vacuo. The residue was purified by column chromatography on silica gel using an elution gradient of hexane:ethyl acetate (95:5 to 50:50) to give the title compound as an oil, that crystallised on standing, (3.15 g, 62%).

¹H nmr (CDCl₃, 400 MHz) δ: 1.36 (s, 9H), 1.83 (m, 2H), 1.97 (m, 2H), 2.22 (s, 3H), 2.62 (m, 1H), 2.98 (m, 2H), 3.80 (s, 3H), 3.98 (m, 4H), 6.94 (m, 3H), 7.04 (m, 2H), 7.17 (d, 1H), 7.23 (m, 1H),

LRMS: m/z 582 (M+23)⁺

Preparation 45

### Methyl 2-[4-(4-(3-tert-butoxyphenyl)-3-methylphenyl)-piperidin-1-ylsulphonyl]-2-methyl-propanoate

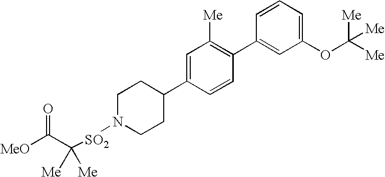

Potassium tert-butoxide (13.63 ml, 1M in tetrahydrofuran, 13.63 mmol) was added dropwise to a solution of the acetate from preparation 44 (2.5 g, 5.45 mmol) and methyl iodide (3.4 ml, 54.5 mmol) in tetrahydrofuran, and once addition was complete, the reaction was stirred at room temperature for 72 hours. The mixture was partitioned between ethyl acetate and water and the layers separated. The organic phase was dried (MgSO₄), filtered and evaporated in vacuo, to give the crude title compound, which was used without further purification (3.1 g).

¹H nmr (CDCl₃, 400 MHz) δ: 1.36 (s, 9H), 1.63 (s, 6H), 1.77-1.94 (m, 4H), 2.22 (s, 3H), 2.63 (m, 1H), 3.05 (m, 2H), 3.80 (s, 3H), 3.95 (m, 2H), 6.90-7.10 (m, 5H), 7.18 (m, 1H), 7.24 (m, 1H).

LRMS: m/z 488 (M+1)⁺

Preparation 46

### Methyl 4-[4-(4-{3-tert-butoxyphenyl}-3-methylphenyl)-piperidin-1-ylsulphonyl]-tetrahydro-2H-pyran-4-carboxylate

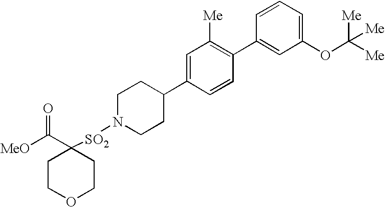

Nitrogen was bubbled through a mixture of cesium fluoride (2.19 g, 14.43 mmol), tri-o-tolyl phosphine (20 mg, 0.065 mmol), tris(dibenzylideneacetone)dipalladium (0) (30 mg, 0.032 mmol) and the bromide from preparation 27 (2.9 g, 6.5 mmol) and the boronic acid from preparation 8 (1.78 g, 9.75 mmol) in anhydrous 1,2-dimethoxyethane (40 ml). The reaction was then heated under reflux under a nitrogen atmosphere for 24 hours. The cooled reaction was partitioned between ethyl acetate and water, the organic phase dried (MgSO₄), filtered and concentrated in vacuo. The residue was triturated with diisopropyl ether, the solid filtered and dried under vacuum, to give the desired product as a cream-coloured solid, (2.0 g, 58%). The filtrate was concentrated in vacuo and the residual oil purified by column chromatography on silica gel using an elution gradient of hexane:dichloromethane:methanol (50:50:0 to 0:100:0 to 0:99:1) to provide an additional (630 mg, 18%) of the title compound. ¹H nmr (CDCl₃, 400 MHz) δ: 1.37 (s, 9H), 1.76-1.92 (m, 4H), 2.20 (m, 5H), 2.40 (m, 2H), 2.60 (m, 1H), 3.02 (m, 2H), 3.29 (m, 2H), 3.86 (m, 5H), 3.98 (m, 2H), 6.94 (m, 3H), 7.02 (m, 2H), 7.14 (m, 1H), 7.22 (m, 1H).

LRMS: m/z 552 (M+23)⁺

Preparation 47

### Methyl 2-[4-(4-{3-hydroxyphenyl}-3-methylphenyl)-piperidin-1-ylsulphonyl]-2-methyl-propanoate

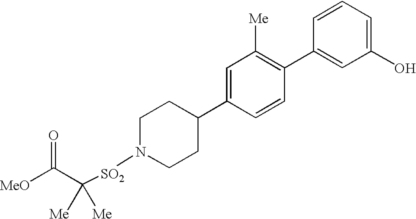

Trifluoroacetic acid (25 ml) was added to a solution of the tert-butoxy ether from preparation 45 (4.8 g, 9.80 mmol) in dichloromethane (50 ml), and the solution stirred for 4 hours. The reaction mixture was concentrated in vacuo, and the residue purified by column chromatography on silica gel, twice using an elution gradient of dichloromethane methanol (10:0 to 95:5) to give the desired product (536 mg, 13%). ¹H nmr (CDCl₃, 400 MHz) δ: 1.62 (s, 6H), 1.76-1.92 (m, 4H), 2.22 (s, 3H), 2.62 (m, 1H), 3.04 (m, 2H), 3.78 (s, 3H), 3.95 (m, 2H), 6.78 (m, 2H), 6.83 (m, 1H), 7.03 (m, 2H), 7.15 (m, 1H), 7.21 (m, 1H).

LRMS: m/z 454 (M+23)⁺

Anal. Found: C, 63.70; H, 6.70; N, 3.20. C₂₃H₂₉NO₅S requires C, 64.01; H, 6.77; N, 3.25%

Preparation 48

### Methyl 4-[4-(4-{3-hydroxyphenyl}-3-methylphenyl)-piperidin-1-ylsulphonyl]-tetrahydro-2H-pyran-4-carboxylate

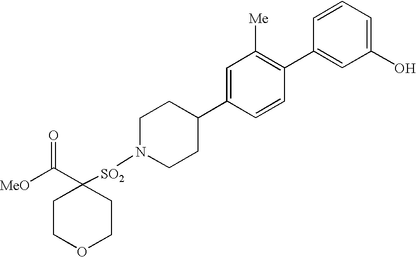

Triethylsilane (2 ml, 13.05 mmol), followed by trifluoroacetic acid (5 ml) were added to an ice-cooled solution of the tert-butyl ether from preparation 46 (2.3 g, 4.35 mmol) in dichloromethane (5 ml) and the reaction stirred for 2 hours. The mixture was concentrated in vacuo, and the residue azeotroped with toluene. The resulting foam was triturated with di-isopropyl ether, filtered and dried to afford the title compound as a solid, (1.94 g, 94%).

Alternative Method

Palladium (II) acetate (300 mg, 1.34 mmol) and triphenylphosphine (708 mg, 2.70 mmol) were suspended in acetone (90 ml), and sonicated for 2 minutes. The suspension was then added to a mixture of 5-bromo-2-iodotoluene (7.9 g, 27 mmol), and the boronic acid from preparation 8 (5.7 g, 29.4 mmol) in aqueous sodium carbonate (42 ml, 2N). The reaction mixture was heated under reflux for 2 hours, then cooled and diluted with water (300 ml). This mixture was extracted with ether (2×250 ml), the combined organic extracts dried (MgSO₄), filtered and evaporated in vacuo. The residue was purified by column chromatography on silica gel using hexane:ether (99:1) as eluant to give 3-(4-bromo-2-methylphenyl)phenyl tert-butyl ether, 7.9 g.

A solution of this intermediate ether (480 mg, 1.5 mmol) in tetrahydrofuran (2 ml), followed by a crystal of iodine, were added to magnesium (45 mg, 1.8 mmol), and the mixture was heated under reflux for 2 hours. The solution was diluted with tetrahydrofuran (3 ml), cooled to −78° C., and a solution of the ketone from preparation 16 (425 mg, 1.4 mmol) in tetrahydrofuran (15 ml) added dropwise.The reacton mixture was stirred at −78° C. for 30 minutes, then allowed to warm to room temperature. Aqueous ammonium chloride was added, the mixture extracted with ethyl acetate (2×50 ml) and the combined organic extracts were dried (MgSO₄), filtered and evaporated in vacuo. The residue was purified by column chromatography on silica gel using pentane:ethyl acetate (50:50) to afford methyl 4-[4-(4-{3-tert-butoxyphenyl}-3-methylphenyl)-4-hydroxypiperidin-1-ylsulphonyl]-tetrahydro-2H-pyran-4-carboxylate as a clear oil, 280 mg.

Triethylsilane (0.5 ml, 3.14 mmol), followed by trifluoroacetic acid (5 ml) were added to a solution of this intermediate (350 mg, 0.64 mmol) in dichloromethane (5 ml), and the reaction stirred at room temperature overnight. The reaction mixture was concentrated in vacuo, the residue azeotroped with toluene and the resulting solid dried under vacuum to afford the title compound, (300 mg).

¹H nmr (CDCl₃, 400 MHz) δ: 1.74-1.90 (m, 4H), 2.20 (m, 5H), 2.40 (m, 2H), 2.62 (m, 1H), 3.02 (m, 2H), 3.29 (m, 2H), 3.87 (m, 5H), 3.98 (m, 2H), 6.77 (m, 2H), 6.83 (d, 1H), 7.02 (m, 2H), 7.15 (d, 1H), 7.21 (m, 1H).

Preparation 49

### Methyl 2-[4-(4-{3-[(2S)-2,3-dihydroxypropoxy]phenyl}-3-methylphenyl)-piperidin-1-ylsulphonyl]-2-

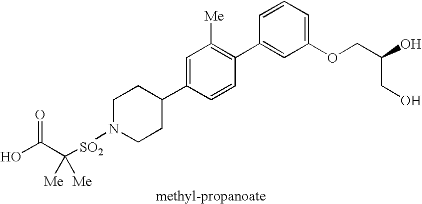

A mixture of the alcohol from preparation 47 (800 mg, 1.86 mmol), S-glycidol (0.12 ml, 1.86 mmol), and triethylamine (10 μl, 0.09 mmol) in methanol (10 ml) was heated under reflux overnight. Tlc analysis showed starting material remaining, so the mixture was concentrated to low volume, and heated under reflux for a further 4 hours. The cooled reaction was evaporated in vacuo and the residue purified by column chromatography on silica gel using an elution gradient of hexane:ethyl acetate (91:9 to 50:50). The desired product was obtained as an oil, that gave a white foam on drying under vacuum, (391 mg, 42%).

¹H nmr (DMSO-d₆, 400 MHz) δ: 1.50 (s, 6H), 1.58 (m, 2H), 1.80 (m, 2H), 2.18 (s, 3H), 2.67 (m, 1H), 3.02 (m, 2H), 3.40 (m, 2H), 3.74 (m, 6H), 3.83 (m, 1H), 3.98 (m, 1H), 4.55 (m, 1H), 4.80 (m, 1H), 6.80 (m, 2H), 6.84 (m, 1H), 7.05 (m, 3H), 7.26 (m, 1H).

LRMS: m/z 528 (M+23)⁺

Preparation 50

### Methyl 4-[4-(4-{3-[1,3-dibenzyloxy-2-propoxy]phenyl)-3-methylphenyl)-piperidin-1-ylsulphonyl]-tetrahydro-2H-pyran-4-carboxylate

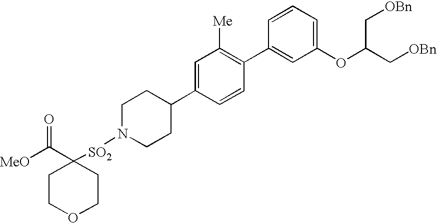

A mixture of the alcohol from preparation 48 (300 mg, 0.63 mmol), diethyl azodicarboxylate (150 μl, 0.95 mmol), triphenylphosphine (250 mg, 0.95 mmol), and 1,3-dibenzyloxy-2-propanol (260 mg, 0.95 mmol) in tetrahydrofuran (6 ml), was stirred at room temperature for 3 hours. Tlc analysis showed some starting material remaining, so additional 1,3-dibenzyloxy-2-propanol (80 mg, 0.3 mmol), triphenyl phosphine (80 mg, 0.3 mmol) and diethyl azodicarboxylate (80 mg, 0.32 mmol) were added, and stirring was continued for an hour. The mixture was evaporated in vacuo, and the residue purified by column chromatography on silica gel using pentane:ethyl acetate (66:34) as eluant to give the title compound as a colourless oil, (400 mg, 87%).

¹H nmr (CDCl₃, 400 MHz) δ: 1.75-1.94 (m, 4H), 2.20 (m, 5H), 2.40 (m, 2H), 2.62 (m, 1H), 3.04 (m, 2H), 3.30 (m, 2H), 3.75 (m, 4H), 3.89 (m, 5H), 3.99 (m, 2H), 4.57 (m, 5H), 6.89 (m, 3H), 7.02 (m, 2H), 7.14 (d, 1H), 7.24 (m, 11H).

Preparation 51

### Methyl 4-[4-(4-{3-[1,3-dihydroxy-2-propoxy]phenyl}-3-methylphenyl)-piperidin-1-ylsulphonyl]-tetrahydro-2H-pyran-4-carboxylate

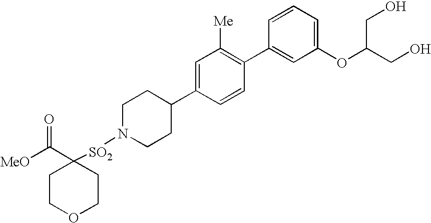

A mixture of the dibenzyl ether from preparation 50 (770 mg, 1.06 mmol), ammonium formate (1.4 g, 11.0 mmol) and palladium hydroxide on carbon (400 mg) in methanol (40 ml) was heated under reflux for 2 hours. Tlc analysis showed some starting material remaining, so additional palladium hydroxide (300 mg) was added, and the reaction was heated under reflux overnight. The cooled mixture was filtered through Arbocel®, and the filtrate evaporated in vacuo. The crude product was purified by column chromatography on silica gel using ethyl acetate:pentane (84:16) as eluant to afford the title compound as a white foam, (375 mg, 65%).

¹H nmr (CDCl₃, 400 MHz) δ: 1.76-1.94 (m, 6H), 2.20 (m, 5H), 2.40 (m, 2H), 2.62 (m, 1H), 3.04 (m, 2H), 3.29 (m, 2H), 3.90 (m, 10H), 3.99 (m, 2H), 6.94 (m, 3H), 7.03 (m, 2H), 7.16 (d, 1H), 7.30 (m, 1H).

Preparation 52

### Methyl 4-[4-(4-{3-[(2R)-2,3-dihydroxypropoxy]phenyl}-3-methylphenyl)-piperidin-1-ylsulphonyl]-tetrahydro-2H-pyran-4-carboxylate

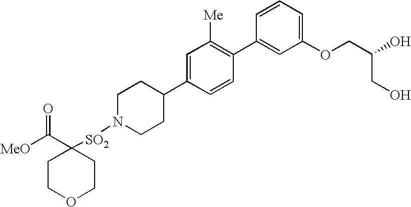

The title compound was obtained (17%) from the compound from preparation 48 and R-glycidol, following a similar procedure to that described in preparation 49.

¹H nmr (CDCl₃, 400 MHz) δ: 1.75-1.97 (m, 4H), 2.20 (m, 5H), 2.40 (m, 2H), 2.61 (m, 1H), 3.02 (m, 2H), 3.28 (m, 2H), 3.58-4.14 (m, 12H), 6.84 (m, 3H), 7.02 (m, 2H), 7.15 (m, 1H), 7.26 (m, 1H).

LRMS: m/z 570 (M+23)⁺

Preparation 53

### Methyl 4-[4-(4-{3-[(2S)-2,3-dihydroxypropoxy]phenyl}-3-methylphenyl)-piperidin-1-ylsulphonyl]-tetrahydro-2H-pyran-4-carboxylate

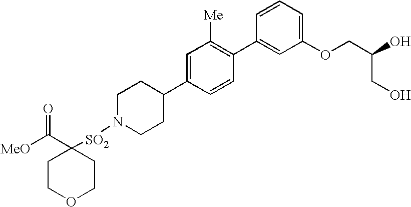

The title compound was obtained as a white solid (52%) after recrystallisation from di-isopropylether, from the alcohol of preparation 48 and S-glycidol, following a similar procedure to that described in preparation 49.

¹H nmr (DMSO-d₆, 300 MHz) δ: 1.50-1.66 (m, 2H), 1.81 (m, 2H), 1.99 (m, 2H), 2.19-2.34 (m, 5H), 2.70 (m, 1H), 3.06 (m, 2H), 3.20 (m, 2H), 3.43 (m, 2H), 3.70-3.98 (m, 9H), 4.00 (dd, 1H), 4.60 (t, 1H), 4.90 (d, 1H), 6.80-6.95 (m, 3H), 7.15 (m, 3H), 7.31 (m, 1H).

LRMS: m/z 570 (M+23)⁺

Preparation 54

### Methyl 2-[4-(4-{3-(2,2-diethoxyethoxy)phenyl}-3-methylphenyl)-piperidin-1-ylsulphonyl]-2-methylpropanoate

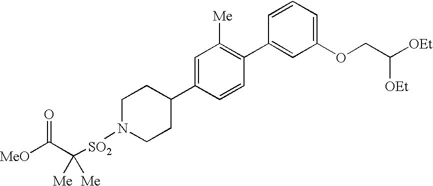

20% Palladium hydroxide on carbon (250 mg) was added to a solution of the 1,2,3,6-tetrahydropyridine from preparation 34 (3.0 g, 5.5 mmol) and ammonium formate (1.04 g, 16.5 mmol) in methanol (70 ml) and 1,4-dioxan (28 ml), and the reaction was stirred at 60° C. for 2 hours. Additional ammonium formate (1.0 g, 15.8 mmol) and palladium hydroxide on carbon (250 mg) were added and stirring was continued for a further 2 hours. The mixture was cooled, filtered through Arbocel®, and the filter pad washed well with methanol. The combined filtrates were evaporated in vacuo and the residue partitioned between water and ether. The layers were separated, the organic phase washed with water, brine, dried (MgSO₄), filtered and evaporated in vacuo to give the title compound as a colourless oil, (2.8 g, 93%).

¹H nmr (CDCl₃, 300 MHz) δ: 1.22 (t, 6H), 1.68 (s, 6H), 1.78-1.96 (m, 4H), 2.25 (s, 3H), 2.64 (m, 1H), 3.08 (m, 2H), 3.60-3.82 (m, 7H), 3.94-4.05 (m, 4H), 4.84 (t, 1H), 6.90 (m, 3H), 7.09 (m, 2H), 7.18 (d, 1H), 7.29 (d, 1H).

Anal. Found: C, 63.43; H, 7.75; N, 2.46. C₂₉H₄₁NO₇S requires C, 63.60; H, 7.55; N, 2.56%.

Preparation 55

### Methyl 4-[4-(4-{3-(2,2-diethoxyethoxy)phenyl-3-methylphenyl)-piperidin-1-ylsulphonyl]-tetrahydro-2H-pyran-4-carboxylate

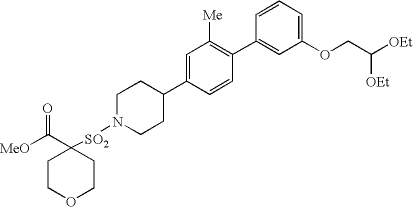

A mixture of cesium fluoride (4.3 g, 28.3 mmol), tri-o-tolyl phosphine (352 mg, 1.15 mmol), tris(dibenzylideneacetone)dipalladium (0) (535 mg, 0.59 mmol) and the boronic acid from preparation 10 (3.89 g, 14.95 mmol) and bromide from preparation 27 (5.0 g, 10.86 mmol) in anhydrous 1,2-dimethoxyethane (70 ml), was heated under reflux for 4½ h. The cooled reaction mixture was concentrated in vacuo to half its volume, then partitioned between water and ethyl acetate. The layers were separated, the aqueous phase extracted with ethyl acetate (3×), and the combined organic U solutions filtered through Arbocel®. The filtrate was washed with brine, dried (Na₂SO₄), filtered and evaporated in vacuo. The residual green oil was purified twice, by column chromatography on silica gel using an elution gradient of dichloromethane:methanol (100:0 to 97:3), then triturated with diisopropyl ether, to afford the title compound as a white solid, (2.38 g, 37%).

¹H nmr (CDCl₃, 400 MHz) δ: 1.20 (t, 6H), 1.76-1.94 (m, 4H), 2.20 (m, 5H), 2.40 (m, 2H), 2.61 (m, 1H), 3.02 (m, 2H), 3.31 (m, 2H), 3.61 (m, 2H), 3.74 (m, 2H), 3.90 (m, 5H), 4.00 (m, 3H), 4.80 (m, 1H), 6.85 (m, 3H), 7.03 (m, 2H), 7.16 (d, 1H), 7.24 (m, 2H).

LRMS: m/z 612 (M+23)⁺

Preparation 56

### Methyl 2-methyl-2-[4-(4-{3-(2-oxoethoxy)phenyl}-3-methylphenyl)-piperidin-1-ylsulphonyl]propanoate

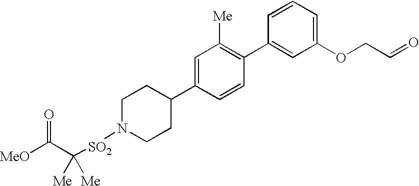

Hydrochloric acid (19 ml, 1N, 19 mmol) was added to a solution of the diethyl ketal from preparation 54 (4.43 g, 8.1 mmol) in acetone (19 ml) and 1,4-dioxan (22 ml), and the reaction stirred at 70° C. for 2 hours. The cooled mixture was neutralised using sodium bicarbonate, concentrated in vacuo, and the residue partitioned between ether and water. The layers were separated, and the organic phase was washed with water, brine, then dried (Na₂SO₄), filtered and evaporated in vacuo. The residue was azeotroped with ethyl acetate, to afford the title compound (quantitative).

¹H nmr (CDCl₃, 300 MHz) δ: 1.67 (s, 6H), 1.78-1.96 (m, 4H), 2.26 (s, 3H), 2.66 (m, 1H), 3.09 (m, 2H), 3.82 (s, 3H), 3.98 (m, 2H), 4.60 (s, 2H), 6.86 (m, 2H), 6.98 (d, 1H), 7.09 (m, 2H), 7.17 (d, 1H), 7.35 (m, 1H), 9.90 (s, 1H).

LRMS: m/z491 (M+18)⁺

Preparation 57

### Methyl 4-[4-(4-3-(2-oxoethoxy)phenyl)-3-methylphenyl)-piperidin-1-ylsulphonyl]-tetrahydro-2H-pyran-4-carboxylate

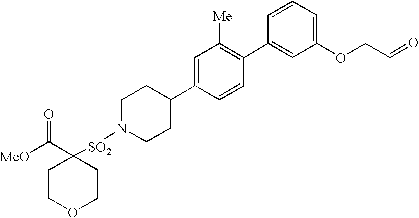

The title compound was obtained as a white foam (quantitative), from the diethyl ketal from preparation 55, following the procedure described in preparation 56.

¹H nmr (CDCl₃, 400 MHz) δ: 1.77-1.93 (m, 4H), 2.21 (m, 5H), 2.40 (d, 2H), 2.62 (m, 2H), 3.02 (m, 2H), 3.30 (m, 2H), 3.88 (m, 5H), 3.99 (m, 2H), 4.57 (s, 2H), 6.83 (m, 2H), 6.94 (d, 1H), 7.02 (m, 2H), 7.15 (d, 1H), 7.30 (m, 1H), 9.83 (s, 1H).

Anal. Found: C, 61.79; H, 6.66; N, 2.46. C₂₇H₃₃NO₇S;0.25CH₃CO₂C₂H₅;0.4H₂O requires C, 61.72; H, 6.62; N, 2.57%.

Preparation 58

### Methyl 2-methyl-2-[4-(4-{3-(2-methylaminoethoxy)phenyl}-3-methylphenyl)-piperidin-1-ylsulphonyl]propanoate

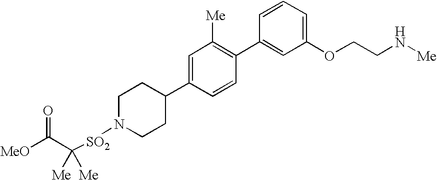

Sodium triacetoxyborohydride (1.5 g, 7.08 mmol) was added portionwise over 1 hour to a solution of the aldehyde from preparation 56 (1.0 g, 2.1 mmol) and methylamine (5.8 ml, 2N in tetrahydrofuran, 11.6 mmol) in dichloromethane (50 ml), and once addition was complete, the reaction was stirred at room temperature overnight. The reaction was partitioned between ethyl acetate and saturated sodium bicarbonate solution, and the layers separated, The organic phase was washed with water, brine, dried (Na₂SO₄), filtered and evaporated in vacuo to give a colourless oil. This was purified by medium pressure column chromatography on silica gel using an elution gradient of dichloromethane:methanol (100:0 to 90:10) to afford the title compound as a foam, (650 mg, 63%).

¹H nmr (CDCl₃, 400 MHz) δ: 1.62 (s, 6H), 1.76-1.90 (m, 4H), 2.22 (s, 3H), 2.56 (s, 3H), 2.61 (m, 1H), 3.04 (m, 4H), 3.78 (s, 3H), 3.95 (m, 2H), 4.12 (t, 2H), 6.83 (m, 3H), 7.03 (m, 2H), 7.14 (d, 1H), 7.24 (m, 1H). Anal. Found: C, 58.39; H, 6.90; N, 4.97. C₂₆H₃₆N₂O₅S;0.75CH₂Cl₂ requires C, 58.17; H, 6.84; N, 5.07%.

Preparations 59 to 63

The compounds of the general formula: 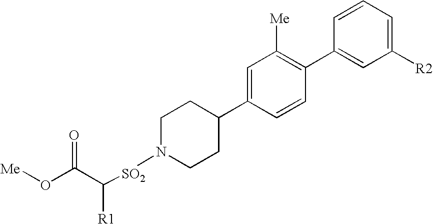
were prepared from the corresponding aldehydes and amines, following similar procedures to those described in preparation 58.
Preparation 64

### Methyl 2-[4-(4-{3-(2-[(N-tert-butoxycarbonyl)(N-methyl)amino]ethoxy)phenyl}-3-methylphenyl)-piperidin-1-ylsulphonyl]-2-methyl-propanoate

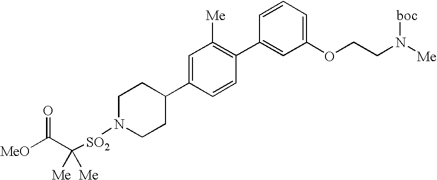

A mixture of the compound from preparation 58 (640 mg, 1.31 mmol), triethylamine (180 μl, 1.30 mol), di-tert-butyl dicarbonate (290 mg, 1.33 mmol) and 4-dimethylaminopyridine (catalytic) in dichloromethane (10 ml) was stirred at room temperature for 3 hours. The reaction mixture was diluted with dichloromethane (50 ml), and washed with water, brine, dried (Na₂SO₄), filtered and evaporated in vacuo. The residual oil was purified by medium pressure column chromatography on silica gel using an elution gradient of pentane:dichloromethane:methanol (100:0:0 to 0:99.5:0.5) to afford the title compound as a gum, (590 mg, 77%).

¹H nmr (CDCl₃, 400 MHz) δ: 1.42 (s, 9H), 1.62 (s, 6H), 1.77-1.90 (m, 4H), 2.22 (s, 3H), 2.63 (m, 1H), 2.97 (s, 3H), 3.03 (m, 2H), 3.58 (m, 2H), 3.78 (s, 3H), 3.95 (m, 2H), 4.08 (m, 2H), 6.82 (m, 3H), 7.04 (m, 2H), 7.16 (d, 1H), 7.25 (m, 1H). LRMS: m/z 611 (M+23)⁺

Anal. Found: C, 60.51; H, 7.19; N, 4.47. C₃₁H₄₄N₂O₇S;0.4CH₂Cl₂ requires C, 60.56; H, 7.25; N, 4.50%.

| PrepNo. | Aldehyde | R1 | R2 | Data |
|---|---|---|---|---|
| 59 | 56 | (Me)₂ | 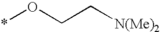 | mp 83-85° C. ¹H nmr (CDCl₃, 400 MHz) δ: 1.62 (s, 6H), 1.78-1.94 (m, 4H), 2.22 (s, 3H), 2.30 (s, 6H), 2.60 (m, 1H), 2.70 t, 2H), 3.02 (m, 2H), 3.79 (s, 3H), 3.96 (m, 2H), 4.06 (t, 2H), 6.83 (m, 3H), 7.02 (m, 2H), 7.15 (d, 1H), 7.22 (m, 1H). LRMS : m/z 503 (M+1)^{+Anal. Found: C, 63.82; H, 7.52; N, 5.45. C}₂₇H₃₈N₂O₅S; 0.1 CH₂Cl₂ requires C, 63.68; |
| #H, 7.53; N, 5.48%. | | | | |
| 60 | 56 | (Me)₂ | 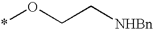 | ¹H nmr (CDCl₃, 400 MHz) δ: 1.66 (s, 6H), 1.59-1.95 (m, 4H), 2.24 (s, 3H), 2.65 (m, 1H), 3.05 (m, 4H), 3.80 (s, 3H), 3.96 (m, 2H), 4.12 (t, 2H), 4.42 (d, 2H), 5.70 (br, s, 1H), 6.85 (m, 3H), 7.07 (m, 2H), 7.17 (d, 1H), 7.24-7.38 (m, 6H). LRMS : m/z 565 (M+1)⁺ |
| 61 | 57 | 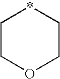 | 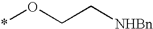 | ¹H nmr (CDCl₃, 400 MHz) ?: 1.75-1.92 (m, 4H), 2.20 (m, 5H), 2.40 (d, 2H), 2.62 (m, 1H), 3.00 (m, 4H), 3.28 (m, 2H), 3.88 (m, 5H), 3.99 (m, 2H), 4.09 (m, 2H), 4.40 (m, 2H), 5.60 (br s, 1H), 6.82 (m, 3H), 7.02 (m, 2H), 7.16 (d, 1H), 7.19-7.35 (m, 6H). LRMS : m/z 607 (M+1)⁺ |
| 62¹ | 30 | 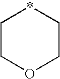 | 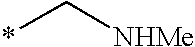 | mp 119-120 ° C. ¹H nmr (CDCl₃, 400 MHz) δ: 1.50 (s, br, 1H), 1.75-1.92 (m, 4H), 2.20 (m, 5H), 2.40 (m, 5H), 2.61 (m, 1H), 3.02 (m, 2H), 3.30 (m, 2H), 3.75-401 (m, 9H), 7.01 (m, 2H), 7.16 (m, 2H), 7.24 (m, 3H). LRMS : m/z 501 (M+1)⁺ |
| 63² | 30 | 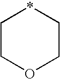 | 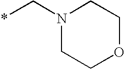 | ¹H nmr (CDCl₃, 400 MHz) δ: 1.75-1.94 (m, 4H), 2.20 (m, 5H), 2.40 (m, 6H), 2.61 (m, 1H), 3.02 (t, 2H), 3.30 (t, 2H), 3.50 (s, 2H), 3.66 (m, 4H), 3.87 (m, 7H), 7.02 (m, 2H), 7.16 (m, 2H), 7.26 (m, 3H). LRMS : m/z 557 (M+1)⁺ |

¹ = purified by crystallisation from ethyl acetate/dichloromethane/di-isopropyl ether.
² = purified by column chromatography on silica gel using ethyl acetate:pentane (75:25) as eluant, and recrystallised from ethyl acetate.

Preparation 65

### Methyl 2-[4-(4-{3-(2-aminoethoxy)phenyl}-3-methylphenyl)-piperidin-1-ylsulphonyl]-2-methyl-propanoate

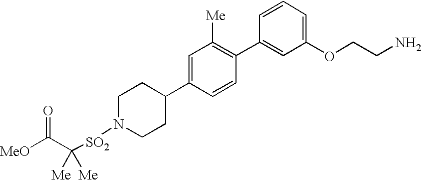

A mixture of the amine from preparation 60 (1.2 g, 2.122 mmol) and 20% palladium hydroxide on carbon (250 mg) in methanol (75 ml), was hydrogenated at 50 psi and room temperature for 18 hours. The reaction mixture was filtered through Arbocel®, and the filter pad washed well with methanol. The combined filtrates were evaporated in vacuo to give an oil. This was purified by medium pressure column chromatography on silica gel using an elution gradient of dichloromethane:methanol (100:0 to 90:10) to afford the title compound (610 mg, 60%).

¹H nmr (CDCl₃, 300 MHz) δ: 1.66 (s, 6H), 1.78-1.97 (m, 4H), 2.28 (s, 3H), 2.66 (m, 1H), 3.10 (m, 4H), 3.82 (s, 3H), 3.99 (m, 4H), 6.88 (m, 3H), 7.10 (m, 2H), 7.19 (d, 1H), 7.30 (m, 1H).

LRMS: m/z475 (M+1)⁺

Anal. Found: C, 61.26; H, 7.09; N, 5.63. C₂₅H₃₄N₂O₅S;0.25dichloromethane requires C, 61.16; H, 7.01; N, 5.65%.

Preparation 66

### Methyl 4-[4-(4-{3-(2-aminoethoxy)phenyl)-3-methylphenyl)-piperidin-1-ylsulphonyl]-tetrahydro-2H-pyran4-carboxylate

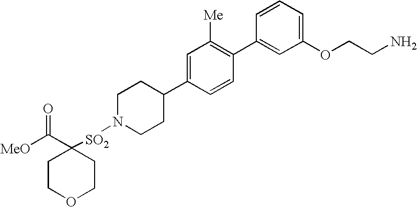

The title compound was obtained as a solid (65%) from the compound from preparation 61, following the procedure described in preparation 65.

¹H nmr (CDCl₃, 400 MHz) δ: 1.76-1.92 (m, 4H), 2.20 (m, 5H), 2.40 (m, 2H), 2.62 (m, 1H), 3.04 (m, 4H), 3.30 (m, 2H), 3.88 (m, 5H), 3.98 (m, 4H), 6.82 (m, 3H), 7.03 (m, 2H), 7.16 (d, 1H), 7.22 (m, 1H).

LRMS: m/z 517 (M+1)⁺

Anal. Found: C, 62.30; H, 6.98; N, 5.40. C₂₇H₃₆N₂O₆S;0.05CH₂Cl₂ requires C, 62.37; H, 6.99; N, 5.38%.

Preparation 67

### Methyl 2-[4-(4-{3-(2-[(tert-butoxycarbonyl)amino]ethoxy)phenyl)-3-methylphenyl)-piperidin-1-ylsulphonyl]-2-methyl-propanoate

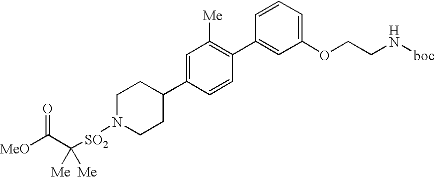

The title compound was obtained as a white foam (69%) from the amine from preparation 65, following a similar procedure to that described in preparation 64.

¹H nmr (CDCl₃, 300 MHz) δ: 1.44 (s, 9H), 1.65 (s, 6H), 1.78-1.95 (m, 4H), 2.25 (s, 3H), 2.64 (m, 1H), 3.08 (m, 2H), 3.55 (m, 2H), 3.81 (s, 3H), 3.97 (m, 2H), 4.04 (t, 2H), 4.99 (br s, 1H), 6.80-6.94 (m, 3H), 7.08 (m, 2H), 7.18 (d, 1H), 7.32 (m, 1H).

LRMS: m/z 597 (M+23)⁺

Anal. Found: C, 62.49; H, 7.46; N, 4.78. C₃₀R₄₂N₂O₇S requires C, 62.69; H, 7.37; N, 4.87%

Preparation 68

### Methyl 4-[4-(4-{3-(2-[(tert-butoxycarbonyl)amino]ethoxy)phenyl}-3-methylphenyl)-piperidin-1-ylsulphonyl]-tetrahydro-2H-pyran-4-carboxylate

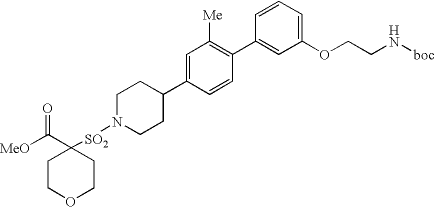

Di-tert-butyl dicarbonate (300 mg, 1.37 mmol) was added to a solution of the amine from preparation 66 (650 mg, 1.26 mmol) in dichloromethane (10 ml), and the reaction stirred at room temperature for 18 hours. The reaction was diluted with dichloromethane (50 ml), then washed with water (2×), brine, then dried (Na₂SO₄), filtered and evaporated in vacuo. The residue was purified by medium pressure column chromatography on silica gel using an elution gradient of dichloromethane:methanol (99.5:0.5 to 99:1) to afford the title compound as a white foam, (710 mg, 91%).

¹H nmr (CDCl₃, 400 MHz) δ: 1.40 (s, 9H), 1.78-1.92 (m, 4H), 2.20 (m, 5H), 2.40 (d, 2H), 2.61 (m, 1H), 3.02 (m, 2H), 3.30 (m, 2H), 3.50 (m, 2H), 3.88 (m, 5H), 4.00 (m, 4H), 4.86 (br s, 1H), 6.82 (m, 3H), 7.02 (m, 2H), 7.15 (d, 1H), 7.05 (m, 1H).

LRMS: m/z 639 (M+23)⁺

Anal. Found: C, 62.15; H, 7.20; N, 4.47. C₃₂H₄₄N₂O₈S requires C, 62.32; H, 7.19; N, 4.54%.

Preparation 69

### Methyl 4-[4-(4-{3-([N-tert-butoxycarbonyl-N-methylamino]methyl)phenyl)-3-methylphenyl)-piperidin-1-ylsulphonyl]-tetrahydro-2H-pyran-4-carboxylate

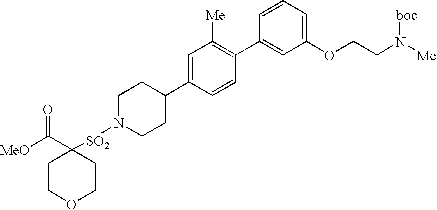

The title compound was prepared from the amine from preparation 62, using a similar procedure to that described in preparation 64. The crude product was purified by column chromatography on silica gel using an elution gradient of ethyl acetate:pentane (25:75 to 50:50) and triturated with di-isopropyl ether to give the title compound as a white solid, (714 mg, 65%).

mp 122-123° C.

¹H nmr (CDCl₃, 400 MHz) δ: 1.42 (s, 9H), 1.75-1.92 (m, 4H), 2.20 (m, 5H), 2.40 (m, 2H), 2.61 (m, 1H), 2.82 (s, 3H), 3.03 (m, 2H), 3.30 (m, 2H), 3.85 (m, 5H), 3.99 (m, 2H), 4.42 (s, 2H), 7.03 (m, 2H). 7.17 (m, 4H), 7.35 (m, 1H).

LRMS: m/z 623 (M+23)⁺

Anal. Found: C, 63.92; H, 7.36; N, 4.57. C₃₂H₄₄N₂O₇S requires C, 63.98; H, 7.38; N, 4.66%

Preparation 70

### 2-[(4-{4-[6-(2-Hydroxyethoxy)pyridin-2-yl]-3-methylphenyl}-piperidin-1-ylsulphonyl]-2-methylpropanoic acid

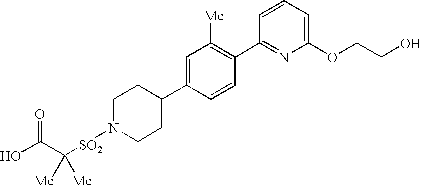

A mixture of the methyl ester from preparation 35 (4.1 g, 8.6 mmol) and aqueous sodium hydroxide (17 ml, 1N, 17.0 mmol) in methanol (50 ml), was heated under reflux for 30 minutes, then cooled. The reaction was concentrated in vacuo, the residue dissolved in water (200 ml), and the solution acidified to pH 4. The resulting precipitate was filtered off, washed with water, dried under vacuum, and recrystallised from ethyl acetate, to afford the title compound as a white solid, (3.15 g, 79%).

¹H nmr (DMSO-d₆, 300 MHz) δ: 1.42-1.70 (m, 8H), 1.80 (m, 2H), 2.37 (s, 3H), 2.70 (t, 1H), 3.06 (m, H), 3.68 (m, 2H), 3.80 (m, 2H), 4.25 (t, 2H), 4.80 (br, s, 1H), 6.77 (d, 1H), 7.06 (d, 1H), 7.17 (m, 2H), 7.35 (d, 1H), 7.77 (m, 1H), 13.38 (br, s, 1H).

Anal. Found: C, 58.35; H, 6.38; N, 5.83. C₂₃H₃₀N₂O₆S;0.5H₂O requires C, 58.85; H, 6.62; N, 5.94%.

Preparation 71

### 2-(4-{4-[6-(2-Methoxyethoxy)pyridin-2-yl]-3-methylphenyl}-piperidin-1-ylsulphonyl)-2-methylpropanoic acid

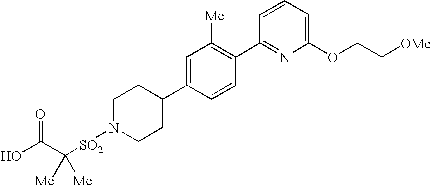

Sodium hydride (60 mg, 60% dispersion in mineral oil, 1.5 mmol) was added to a solution of the methyl ester from preparation 35 (300 mg, 0.63 mmol) in tetrahydrofuran (10 ml), and the solution stirred for 15 minutes. Methyl iodide (200 μl, 3.3 mmol) was added and the reaction heated under reflux for 45 minutes. Aqueous sodium hydroxide solution (2 ml, 1N, 2.0 mmol) and methanol (5 ml) were then added, and the mixture heated under refux for a further 30 minutes. The reaction mixture was cooled to room temperature, diluted with water (20 ml), and acidified to pH 4. This solution was extracted with dichloromethane (3×30 ml), the combined organic extracts dried (Na₂SO₄), filtered and evaporated in vacuo to afford the title compound as a pale yellow foam, (quantitative).

mp 142-146° C.

¹H nmr (CDCl₃, 300 MHz) δ: 1.68 (s, 6H), 1.78-1.96 (m, 4H), 2.41 (s, 3H), 2.66 (m, 1H), 3.09 (m, 2H), 3.43 (s, 3H), 3.78 (t, 2H), 4.00 (m, 2H), 4.52 (t, 2H), 6.78 (d, 1H), 6.98 (d, 1H), 7.08 (m, 2H), 7.38 (d, 1H), 7.61 (d, 1H).

LRMS: m/z 433 (M-CO₂)⁺

Preparation 72

### 4-[4-(4-{6-[2-Hydroxyethoxy]pyridin-2-yl}-3-methylphenyl)piperidin-1-ylsulphonyl]tetrahydro-2H-pyran4-carboxylic acid

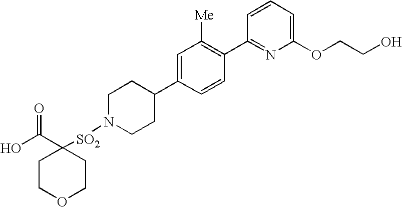

Aqueous sodium hydroxide (5.56 ml, 1N, 5.56 mmol) was added to a solution of the methyl ester from preparation 36 (720 mg, 1.39 mmol) in methanol (20 ml), and the reaction heated under reflux for 3 hours, and stirred for a further 18 hours, at room temperature. The mixture was concentrated in vacuo to remove the methanol, and the solution acidified to pH 4 using acetic acid solution. This was extracted with ethyl acetate (3×), the combined organic extracts washed with brine, dried (MgSO₄), filtered and evaporated in vacuo. The residual solid was recrystallised from ethyl acetate/di-isopropyl ether to afford the title compound as a solid, (517 mg, 74%).

¹H nmr (DMSO-d₆, 300 MHz) δ: 1.62 (m, 2H), 1.82 (m, 2H), 1.98 (m, 2H), 2.24 (m, 2H), 2.36 (s, 3H), 2.74 (m, 1H), 3.09 (t, 2H), 3.22 (m, 2H), 3.64-3.82 (m, 4H), 3.94 (dd, 2H), 4.28 (t, 2H), 4.80 (br s, 1H), 6.78 (d, 1H), 7.06 (d, 1H), 7.16 (m, 2H), 7.36 (d, 1H), 7.78 (m, 1H), 13.82 (br s, 1H).

LRMS: m/z 527(M+18)⁺

Preparation 73

### 4-[4-(4-{6-[(2S)-2,3-dihydroxy-1-propoxy]pyridin-2-yl}-3-methylphenyl)piperidin-1-ylsulphonyl]-tetrahydro-2H-pyran-4-carboxylic acid

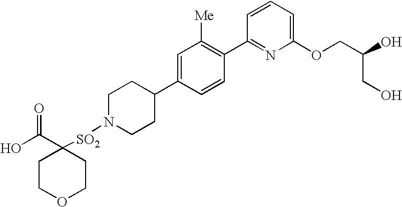

Aqueous sodium hydroxide (3.5 ml, 1M, 3.5 mmol) was added to a solution of the methyl ester from preparation 39 (640 mg, 1.17 mmol) in methanol (15 ml) and 1,4-dioxan (15 ml), and the reaction heated under reflux for 2 hours. Tlc analysis showed starting material remaining, so additonal sodium hydroxide (2 ml, 1M, 2 mmol) was added and the reaction heated under reflux for a further 3 hours. The cooled reaction mixture was concentrated under reduced pressure, the residue dissolved in water, and the pH adjusted to 4 using hydrochloric acid (2N). The resulting precipitate was filtered and dried, and the filtrate extracted with dichloromethane (2×). The combined organic extracts were dried (MgSO₄), filtered and evaporated in vacuo, and the product combined with the filtered solid. This was recrystallised from dichloromethane/ethyl acetate twice, to yield the title compound as a white solid, (579 mg, 92%).

¹H nmr (DMSO-d₆, 400 MHz) δ: 1.60 (m, 2H), 1.80 (m, 2H), 1.92 (m, 2H), 2.23 (d, 2H), 2.34 (s, 3H), 2.66 (m, 1H), 3.08 (m, 2H), 3.17-3.42 (m, 3H), 3.78 (m, 3H), 3.88 (m, 2H), 4.14 (dd, 1H), 4.26 (dd, 1H), 4.60 (br, s, 1H), 4.85 (br, s, 1H), 6.76 (d, 1H), 7.04 (d, 1H), 7.15 (m, 2H), 7.34 (m, 2H), 7.74 (dd, 1H).

LRMS: m/z 557 (M+23)⁺

Preparation 74

### 4-[4-(4-{6-[(2R)-2,3-dihydroxy-1-propoxy]pyridin-2-yl)-3-methylphenyl)piperidin-1-ylsulphonyl]-tetrahydro-2H-pyran-4-carboxylic acid

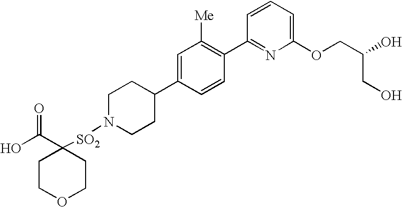

The title compound was obtained as a white solid (87%) from the methyl ester of preparation 40, following a similar procedure to that described in preparation 73.

¹H nmr (DMSO-d₆, 300 MHz) δ: 1.61 (m, 2H), 1.80 (m, 2H), 1.96 (m, 2H), 2.24 (m, 2H), 2.36 (s, 3H), 2.70 (m, 1H), 3.06 (m, 2H), 3.14-3.44 (m, 4H), 3.78 (m, 3H), 3.93 (m, 2H), 4.14 (m, 1H), 4.26 (m, 1H), 4.59 (m, 1H), 4.84 (m, 1H), 6.76 (d, 1H), 7.06 (d, 1H), 7.15 (m, 2H), 7.35 (d, 1H), 7.76 (m, 1H), 13.80 (br, s, 1H).

LRMS: m/z 557 (M+23)⁺

Preparation 75

### 4-[4-(4-{6-[2-Hydroxyethoxy]pyridin-2-yl}-3-methylphenyl)piperidin-1-ylsulphonyl-1-methylpiperidine-4-carboxylic acid

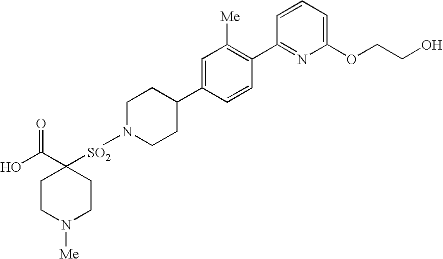

A mixture of the methyl ester from preparation 42 (200 mg, 0.38 mmol) and aqueous sodium hydroxide (1.5 ml, 1N, 1.5 mmol) in methanol (8 ml) and 1,4-dioxan (8 ml) was heated under reflux overnight. The cooled reaction was concentrated in vacuo, the residue acidified to pH 4 using acetic acid, and extraction with ethyl acetate attempted. A precipitate formed in the organic layer, that was filtered off, and combined with the residual solid in the separating funnel, to provide the desired compound as a white powder, (quantitative).

LRMS: m/z 518 (M+1)⁺

Preparation 76

### 1-(tert-Butoxycarbonyl)-4-[4-(4-{6-[2-hydroxyethoxy]pyridin-2-yl}-3-methylphenyl)piperidin-2-ylsulphonyl]-piperidine-4-carboxylic acid

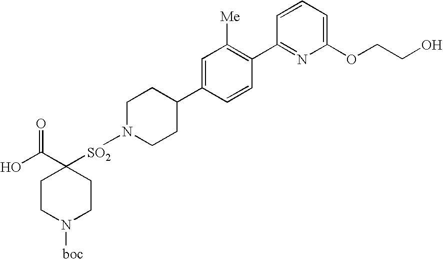

The title compound was obtained as a white solid (87%), from the methyl ester from preparation 43, following a similar procedure to that described in preparation 75.

mp 148-149° C.

¹H nmr (CDCl₃, 300 MHz) δ: 1.42 (s, 9H), 1.80 (m, 4H), 2.00 (m, 2H), 2.36 (s, 3H), 2.41 (m, 2H), 2.58-2.79 (m, 4H), 3.02 (m, 4H), 3.92 (m, 5H), 4.44 (m, 2H), 6.76 (m, 1H), 6.99 (m, 1H), 7.07 (m, 2H), 7.34 (m, 1H), 7.65 (m, 1H).

Preparation 77

### 2-[4-(4-{3-[(2S)-2,3-Dihydroxy-1-propoxy]phenyl}-3-methylphenyl)-piperidin-1-ylsulphonyl]-2-methyl-propanoic acid

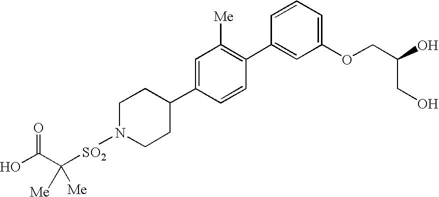

Aqueous sodium hydroxide (1.55 ml, 1M, 1.55 mmol) was added to a solution of the methyl ester from preparation 49 (391 mg, 0.77 mmol) in methanol (5 ml), and the reaction stirred at room temperature overnight. The mixture was partitioned between ethyl acetate and hydrochloric acid (2N), and the phases separated. The organic layer was dried (MgSO₄), filtered and concentrated in vacuo. The residual solid was triturated with di-isopropyl ether, filtered and dried under vacuum, to give the title compound as a white solid, (320 mg, 85%).

¹H nmr (DMSO-d₆, 400 MHz) δ: 1.48 (s, 6H), 1.59 (m, 2H), 1.79 (m, 2H), 2.18 (s, 3H), 2.64 (m, 1H), 3.04 (m, 2H), 3.40 (m, 2H), 3.78 (m, 3H), 3.82 (m, 1H), 3.98 (m, 1H), 4.57 (br, s, 1H), 4.82 (br, s, 1H), 6.80 (m, 2H), 6.85 (m, 1H), 7.05 (m, 2H), 7.12 (m, 1H), 7.27 (m, 1H), 13.25 (br, s, 1H).

Anal. Found: C, 60.77; H, 6.89; N, 2.78. C₂₅H₃₃NO₇S requires C, 61.08; H, 6.77; N, 2.85%.

Preparation 78

### 4-[4-(4-{3-[2,3-dihydroxy-2-propoxy]phenyl}-3-methylphenyl)-piperidin-1-ylsulphonyl]-tetrahydro-2H-pyran-4-carboxylic acid

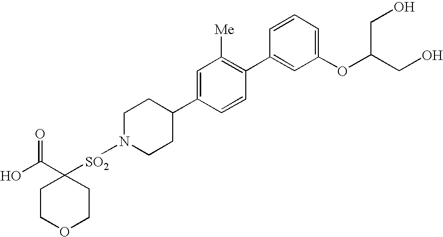

A mixture of the methyl ester from preparation 51 (370 mg, 0.68 mmol), aqueous sodium hydroxide (3 ml, 1M, 3 mmol) in methanol (5 ml) and 1,4-dioxan (5 ml), was heated under reflux for 6 hours. The cooled reaction was concentrated in vacuo, and then diluted with water. This aqueous solution was acidified to pH 2 using hydrochloric acid (2N), and the resulting precipitate filtered, washed with water and dried under vacuum, to give the desired product (270 mg, 74%).

¹H nmr (DMSO-d₆, 400 MHz) δ: 1.60 (m, 2H), 1.79 (m, 2H), 1.95 (m, 2H), 2.19 (m, 5H), 2.63 (m, 1H), 3.02 (m, 4H), 3.56 (m, 4H), 3.76 (m, 2H), 3.88 (m, 2H), 4.22 (m, 1H), 4.68 (m, 2H), 6.78-6.95 (m, 3H), 7.08 (m, 3H), 7.25 (m, 1H).

Preparation 79

### 4-[4-(4-{3-[(2R)-2,3-Dihydroxy-1-propoxy]phenyl}-3-methylphenyl)-piperidin-1-ylsulphonyl]-tetrahydro-(2H)-pyran-4-carboxylic acid

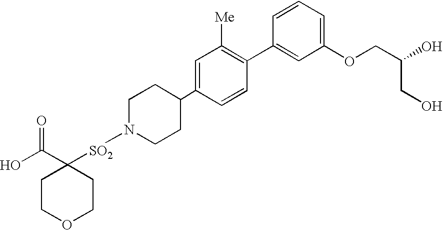

A mixture of the methyl ester from preparation 52 (110 mg, 0.20 mmol), aqueous sodium hydroxide (1 ml, 1M, 1 mmol) in methanol (5 ml) and 1,4-dioxan (5 ml) was heated under reflux for 2 hours. The cooled reaction was evaporated in vacuo, the residue dissolved in water and acidified to pH 1 using hydrochloric acid (1N). The resulting precipitate was filtered, the solid washed with water, and dried under vacuum to give the title compound (91 mg, 85%) as a white solid.

¹H nmr (DMSO-d₆, 400 MHz) δ: 1.60 (m, 2H), 1.80 (m, 2H), 1.94 (m, 2H), 2.20 (m, 5H), 2.65 (m, 1H), 3.05 (m, 2H), 3.18-3.48 (m, 4H), 3.77 (m, 3H), 3.88 (m, 3H), 4.00 (m, 1H), 6.81 (m, 2H), 6.89 (m, 1H), 7.10 (m, 3H), 7.30 (m, 1H).

LRMS: m/z 556 (M+23)⁺

Preparation 80

### 4-[4-(4-{3-[(2S)-2,3-Dihydroxy-1-propoxy]phenyl}-3-methylphenyl)-piperidin-1-ylsulphonyl]-tetrahydro-(2H)-pyran-4-carboxylic acid

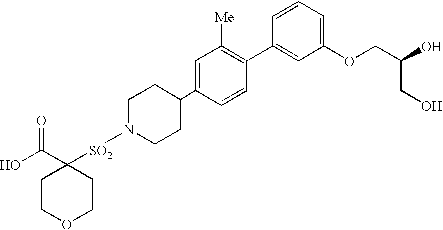

The title compound was obtained as a solid (66%) from the methyl ester from preparation 53, following the procedure described in preparation 79.

¹H nmr (DMSO-d₆, 400 MHz) δ: 1.60 (m, 2H), 1.80 (m, 2H), 1.96 (m, 2H), 2.22 (m, 5H), 2.68 (m, 1H), 3.06 (m, 2H), 3.21 (m, 2H), 3.42 (d, 2H), 3.78 (m, 3H), 3.90 (m, 3H), 4.00 (m, 1H), 6.81 (m, 2H), 6.90 (d, 1H), 7.12 (m, 3H), 7.31 (dd, 1H).

Preparation 81

### 2-[4-(4-{3-(2-[N-tert-Butoxycarbonyl-N-methylamino]ethoxy)phenyl-3-methylphenyl)-piperidin-1-ylsulphonyl]-2-methylpropanoic acid

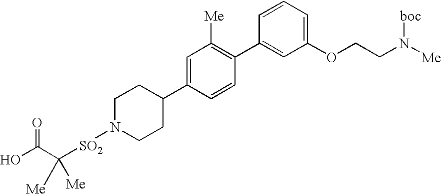

A mixture of the methyl ester from preparation 64 (540 mg, 0.92 mmol), and aqueous sodium hydroxide (6 ml, 1N, 6.0 mmol) in 1,4-dioxan (2.3 ml) and methanol (6 ml) was heated under reflux for 3½ h. The cooled mixture was concentrated in vacuo to remove the organic solvents, and the residual aqueous solution was acidified to pH 4 using acetic acid. This was extracted with ethyl acetate (2×), the combined organic extracts washed with water, brine, dried (Na₂SO₄), filtered and evaporated in vacuo. The residue was azeotroped with toluene, then ethyl acetate, and finally dichloromethane, to afford the title compound as a white foam, (520 mg, 98%).

¹H nmr (CDCl₃, 400 MHz) δ: 1.41 (s, 9H), 1.64 (s, 6H), 1.78-1.94 (m, 4H), 2.22 (s, 3H), 2.63 (m, 1H), 2.97 (s, 3H), 3.06 (m, 2H), 3.59 (m, 2H), 3.98 (m, 2H), 4.08 (t, 2H), 6.83 (m, 3H), 7.04 (m, 2H), 7.16 (d, 1H),7.26 (m, 1H).

LRMS: m/z 597 (M+23)⁺

Anal. Found: C, 61.17; H, 7.27; N, 4.65. C₃₀H₄₂N₂O₇S;0.2CH₂Cl₂ requires C, 61.30; H, 7.22; N, 4.73%.

Preparations 82 to 86

The compounds of the general formula:

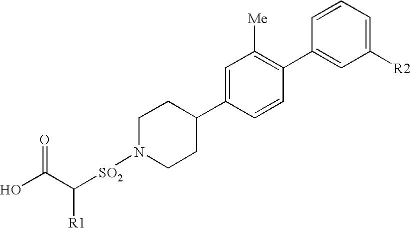

were prepared from the corresponding methyl esters, following similar procedures to those described in preparation 81.

| Prep | Starting | | | |
|---|---|---|---|---|
| No. | ester | R1 | R2 | Data |
| 82 | 67 | (Me)₂ | 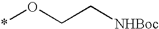 | ¹H nmr (DMSO-d₆, 300 MHz) δ: 1.36 (s, 9H), 1.50 (s, 6H), 1.62 (m, 2H), 1.81 (m, 2H), 2.20 (s, 3H), 2.68 (m, 1H), 3.06 (m, 2H), 3.28 (m, 4H), 3.80 (m, 2H), 3.98 (t, 2H), 6.80-6.99 (m, 3H), 7.14 (m, 2H), 7.30 (m, 1H). LRMS : m/z 583 (M+23)^{+Anal. Found: C, 58.94; H, 7.02; N, 4.64. C}₂₉H₄₀N₂O₇S;0.4CH₂Cl₂ requires C, 59.02; H, 6.94; N, 4.68%. |
| 83¹ | 59 | (Me)₂ | 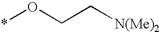 | mp 230-232° C. ¹H nmr (DMSO-d₆, 400 MHz) δ: 1.46 (s, 6H), 1.60 (m, 2H), 1.80 (m, 2H), 2.18 (s, 3H), 2.25 (s, 6H), 2.64 (m, 3H), 3.02 (m, 2H), 3.78 (m, 2H), 4.06 (t, 2H), 6.80 (m, 2H), 6.86 (d, 1H), 7.08 (m, 2H), 7.28 (dd, 1H). Anal. Found: C, 62.70; H, 7.37; N, 5.53. C₂₆H₃₆N₂O₅S;0.5H₂O requires C, 61.32; H, 7.05; N, 4.61%. |
| 84 | 68 | 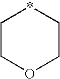 | 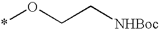 | mp 194-196° C. ¹H nmr (CDCl₃, 400 MHz) δ: 1.42 (s, 9H), 1.75-1.92 (m, 4H), 2.22 (m, 5H), 2.38 (d, 2H), 2.61 (m, 1H), 3.06 (m, 2H), 3.40 (m, 2H), 3.50 (m, 2H), 3.98 (m, 6H), 6.82 (m, 3H), 7.02 (m, 2H), 7.14 (d, 1H), 7.23 (m, 1H). Anal. Found: C, 61.20; H, 7.05; N, 4.60. C₃₁H₄₂N₂O₈S;0.25H₂O requires C, 61.32; H, 7.05; N, 4.61%. |
| 85² | 69 | 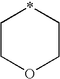 | 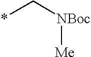 | mp 196-197° C. ¹H nmr (DMSO-d₆, 400 MHz) δ: 1.38 (S, 9H), 1.60 (m, 2H), 1.80 (m, 2H), 1.95 (m, 2H), 2.19 (s, 3H), 2.20 (m, 2H), 2.64 (m, 1H), 2.76 (s, 3H), 3.02 (t, 2H), 3.18 (m/t, 2H), 3.77 (m, 2H), 3.86 (m, 2H), 4.38 (s, 2H), 7.12 (m, 6H), 7.37 (m, 1H). LRMS : m/z 609 (M+23)⁺ |
| 86³ | 63 | 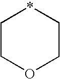 | 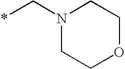 | ¹H nmr (DMSO-d₆, 400 MHz) δ: 1.59 (m, 2H); 1.80 (m, 2H), 1.90 (m, 2H), 2.20 (m, 6H), 2.62-2.79 (m, 4H), 3.00-3.22 (m, 6H), 3.65 (m, 4H), 3.76 (m, 2H), 3.88 (m, 2H), 7.12 (m, 4H), 7.25 (m, 1H), 7.39 (m, 2H) LRMS : m/z 543 (M+1)⁺ |

¹ = isolated by filtration from aqueous acetic acid solution.
² = recrystallised from ethyl acetate/methanol
³ = triturated with di-isopropyl ether

Preparation 87

### N-Hydroxy 1-(tert-butoxycarbonyl)-4-{[4-(4-{6-[2-hydroxyethoxy]pyridin-2-yl}-3-methylphenyl)piperidin-1-yl]sulphonyl}-piperidine-4-carboxamide

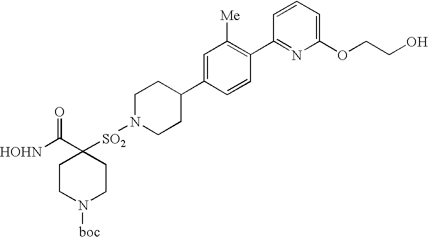

Chlorotrimethylsilane (70 μl, 0.55 mmol) was added to a solution of the acid from preparation 76 (300 mg, 0.50 mmol) in dichloromethane (4 ml), and pyridine (2 ml), and the solution stirred at room temperature under a nitrogen atmosphere for 1 hour. 1-(3-Dimethylaminopropyl)-3-ethylcarbodiimide hydrochloride (115 mg, 0.60 mmol) and 1-hydroxy-7-azabenzotriazole (75 mg, 0.55 mmol) were added, and stirring was continued for a further hour. Hydroxylamine hydrochloride (104 mg, 150 mmol) was added and the reaction stirred at room temperature overnight. The reaction mixture was diluted with water, the solution acidified to pH 1 using hydrochloric acid (2M), then extracted with ethyl acetate. The combined organic solutions were washed with brine, dried (MgSO₄), filtered and evaporated in vacuo. The residue was triturated with ethyl acetate, the resulting precipitate filtered and the filtrate evaporated in vacuo. The residue was recrystallised from ethyl acetate to afford the title compound (148 mg, 48%) as a white solid.

mp 180-181° C.

¹H nmr (DMSO-d₆, 400 MHz) δ: 1.39 (s, 9H), 1.55-1.81 (m, 6H), 2.36 (s, 3H), 2.42 (m, 2H), 2.62 (m, 3H), 3.03 (m, 2H), 3.70 (m, 4H), 3.95 (m, 2H), 4.24 (t, 2H), 4.78 (br, 1H ), 6.75 (d, 1H), 7.04 (d, 1H), 7.15 (m, 2H), 7.34 (d, 1H), 7.75 (m, 1H), 9.16 (s, 1H), 11.00 (s, 1H).

LRMS: m/z 617 (M−1)⁺

Preparation 88

### N-Hydroxy 2-[4-(4-{3-(2-[(N-tert-butoxycarbony-N-methyl)amino]ethoxy)phenyl}-3-methylphenyl)-piperidin-1-ylsulphonyl]-2-methylpropanamide

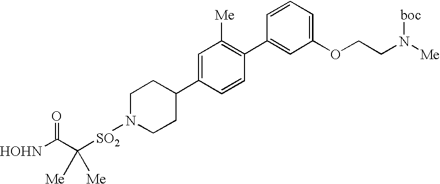

O-(7-Azabenzotriazol-1-yl)-N,N,N′N′-tetramethyluronium hexafluorophosphate (540 mg, 1.42 mmol) was added to a solution of the acid from preparation 81 (520 mg, 0.90 mmol) and N-ethyldiisopropylamine (193 μl, 1.12 mmol) in N-methylpyrrolidinone (10 ml), and the reaction stirred at room temperature under a nitrogen atmosphere for 40 minutes. Hydroxylamine hydrochloride (210 mg, 3.02 mmol) and additional N-ethyldiisopropylamine (730 μl, 4.23 mmol) were added, and the reaction stirred at room temperature overnight. The mixture was partitioned between ethyl acetate and pH 7 buffer solution, and the layers separated. The organic phase was washed consecutively with water, brine, then dried (NaSO₄), filtered and evaporated in vacuo. The crude product was purified by medium pressure column chromatography on silica gel using an elution gradient of dichloromethane:methanol (99.5:0.5 to 98:2 to 80:20) to afford the title compound, (180 mg, 34%).

¹H nmr (CDCl₃, 400 MHz) δ: 1.40 (s, 9H), 1.63 (s, 6H), 1.78 (m, 2H), 1.86 (m, 2H), 2.22 (s, 3H), 2.61 (m, 1H), 2.97 (s, 3H), 3.03 (m, 2H), 3.58 (m, 2H), 3.94 (m, 2H), 4.08 (m, 2H), 6.60 (s, 1H), 6.64 (m, 2H), 7.02 (m, 2H), 7.17 (d, 1H), 7.26 (dd, 1H), 8.99 (s, 1H), 10.75 (s, 1H).

Anal. Found: C, 60.96; H, 7.33; N, 7.11. C₃₀H₄₃N₃O₇S requires C, 61.10; H, 7.35; N, 7.12%.

Preparation 89

### N-Hydroxy 2-[4-(4-{3-(2-[(tert-butoxycarbonyl)amino]ethoxy)phenyl}-3-methylphenyl)-piperidin-1-ylsulphonyl]-2-methylpropionamide

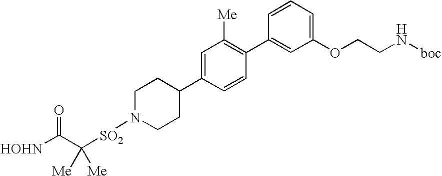

The title compound was obtained (49%) from the acid from preparation 82, following a similar procedure to that described in preparation 88.

¹H nmr (DMSO-d₆, 400 MHz) δ: 1.37 (s, 9H), 1.48 (s, 6H), 1.60 (m, 2H), 1.79 (m, 2H), 2.20 (s, 3H), 2.64 (m, 1H), 3.04 (m, 2H), 3.28 (m, 2H), 3.75 (m, 2H), 3.98 (t, 2H), 6.80-6.98 (m, 4H), 7.10 (s, 2H), 7.15 (s, 1H), 7.30 (dd, 1H), 8.99 (s, 1H), 10.55 (s, 1H).

LRMS: m/z 598 (M+23)⁺

Anal. Found: C, 59.25; H, 7.09; N, 7.38. C₂₉H₄₁N₃O₇S;0.1CH₂Cl₂ requires C, 59.83; H, 7.11; N, 7.19%

Preparation 90

### N-Hydroxy 4-[4-(4-{3-(2-[(N-tert-butoxycarbonyl)amino]ethoxy)phenyl}-3-methylphenyl)-piperidin-1-ylsulphonyl)-tetrahydro-2H-pyran-4-carboxamide

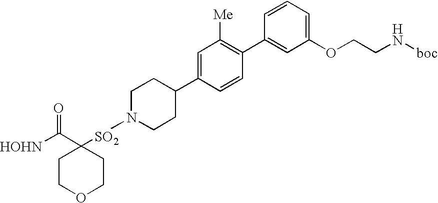

1-(3-Dimethylaminopropyl)-3-ethylcarbodiimide hydrochloride (260 mg, 1.36 mmol) and 1-hydroxy-7-azabenzotriazole (150 mg, 1.1 mmol) were added to a solution of the acid from preparation 84 (620 mg, 1.03 mmol) in pyridine (2 ml) and dichloromethane (6 ml), and the mixture stirred at room temperature for 30 minutes. Hydroxylamine hydrochloride (155 mg, 2.25 mmol) was added and the reaction stirred at room temperature for 18 h. The reaction mixture was partitioned between ethyl acetate and pH 7 buffer solution, and the layers separated. The aqueous phase was extracted with ethyl acetate, the combined organic solutions washed again with pH 7 buffer solution, then brine, dried (Na₂SO₄), filtered and evaporated in vacuo. The residue was azeotroped with toluene, and then purified by medium pressure column chromatography on silica gel using an elution gradient of dichloromethane:methanol (100:0 to 90:10). The product was recrystallised from ethyl acetate/pentane to afford the title compound as a solid, (340 mg, 53%).

mp 181-182° C.

¹H nmr (DMSO-d₆, 400 MHz) δ: 1.35 (s, 9H), 1.60 (m, 2H), 1.78 (m, 2H), 1.90 (m, 2H), 2.19 (s, 3H), 2.28 (m, 2H), 2.61 (m, 1H), 3.02 (m, 2H), 3.20 (m, 2H), 3.22 (m, 2H), 3.70 (m, 2H), 3.84 (m, 2H), 3.98 (t, 2H), 6.79-6.95 (m, 4H), 7.08 (s, 2H), 7.15 (s, 1H), 7.28 (m, 1H), 9.10(s, 1H), 10.93 (s, 1H).

LRMS: m/z 640 (M+23)⁺

Anal. Found: C, 60.27; H, 7.04; N, 6.63. C₃₁H₄₃N₃O₈S requires C, 60.27; H, 7.02; N, 6.88%.

Preparation 91

### N-Hydroxy 4-[4-(4-{3-(N-tert-butoxycarbonyl-N-methyl)aminomethyl)phenyl}-3-methylphenyl)-piperidin-1-ylsulphonyl]-tetrahydro-2H-pyran-4-carboxamide

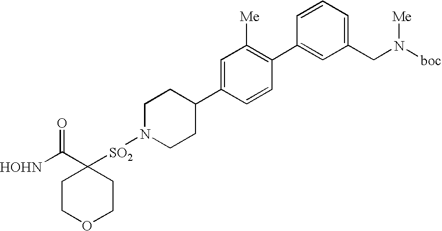

1-(3-Dimethylaminopropyl)-3-ethylcarbodiimide hydrochloride (216 mg, 1.12 mmol) and 1-hydroxy-7-azabenzotriazole (128 mg, 0.94 mmol) were added to a solution of the acid from preparation 85 (550 mg, 0.94 mmol) in pyridine (2 ml) and N,N dimethylformamide (6 ml), and the mixture stirred at room temperature for 1 hour. Hydroxylamine hydrochloride (195 mg, 2.82 mmol) was added and the reaction stirred at room temperature overnight. The reaction mixture was partitioned between ethyl acetate and pH 7 buffer solution, and the layers separated. The aqueous phase was extracted with ethyl acetate (×2), the combined organic solutions washed with 2N hydrochloric acid, dried (MgSO₄), filtered and evaporated in vacuo. The residue was crystallised from methanol/ethyl acetate to afford the title compound as a solid, (393 mg, 70%).

¹H nmr (DMSO-d₆, 400 MHz) δ: 1.36 (s, 9H), 1.59 (m, 2H), 1.78 (m, 2H), 1.88 (m, 2H), 2.18 (s, 3H), 2.27 (m, 2H), 2.61 (m, 1H), 2.76 (s, 3H), 3.00 (m, 2H), 3.18 (m, 2H), 3.68 (m, 3.82 (m, 2H), 4.38 (s, 2H), 7.09 (m, 3H), 7.18 (m, 3H), 7.38 (m, 1H), 9.10 (s, 1H), 10.92 (s, 1H).

LRMS: m/z 624 (M+1)⁺

Preparation 92

### 1-(4-Bromo-2-methylphenyl)-1H-pyrazol-3-ol

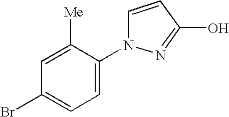

Potassium tert-butoxide (20 ml, 1M in tert-butanol, 20.0 mmol) was added to 1-(4-bromo-2-methylphenyl)hydrazine (J.Chem.Soc. 109; 1916; 582)(2.01 g; 10.0 mmol) to give a dark brown suspension. Ethyl propiolate (1.02 ml, 10 mmol) was then added dropwise over 10 minutes, with cooling, and once addition was complete, the reaction was heated under reflux for 4 hours. The reaction was diluted with water (200 ml) and this mixture washed with dichloromethane (2×50 ml). The aqueous phase was acidified using hydrochloric acid (2N), extracted with dichloromethane (5×100 ml), these combined organic extracts dried (MgSO₄), filtered and evaporated in vacuo. The crude product was purified by column chromatography on silica gel using dichloromethane:methanol (98:2) as eluant, and triturated with ether/di-isopropyl ether to give the title compound (615 mg, 24%) as a solid.

mp 208-210° C.

¹H nmr (DMSO-d₆, 400 MHz) δ: 2.26 (s, 3H), 5.75 (s, 1H), 7.22 (d, 1H), 7.44 (d, 1H), 7.57 (s, 1H), 7.74 (s, 1H), 10.00 (s, 1H).

LRMS: m/z 253, 255 (M+1)⁺

Anal.Found: C, 47.31; H, 3.52; N, 10.99. C₁₀H₉BrN₂O requires C, 47.46; H, 3.58; N, 11.07%.

Preparation 93

### 1-(4-Bromo-2-methylphenyl)-3-methoxy-1H-pyrazole

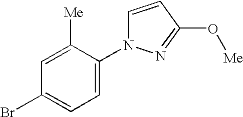

A mixture of the pyrazole from preparation 92 (1.52 g, 6.0 mmol), potassium carbonate (828 mg, 6.0 mmol), and dimethylsulphate (624 ml, 6.6 mmol) in 1-methyl-2-pyrrolidinone (15 ml) was heated at 90° C. for 5 hours. Tlc analysis showed starting material remaining, so additional potassium carbonate (828 mg, 6.0 mmol) and dimethylsulphoxide (624?l, 6.6 mmol) were added, and stirring continued at 90° C. for a further 18 hours. The cooled reaction was poured into water (200 ml), and the mixture extracted with ethyl acetate (3×100 ml). The combined organic extracts were washed with brine (3×100 ml), dried (MgSO₄), filtered and concentrated in vacuo. The residue was purified by column chromatography on silica gel using dichloromethane as the eluant, to give the desired product as a pale yellow oil, (970 mg, 61%).

¹H nmr (CDCl₃, 400 MHz) δ: 2.30 (s, 3H), 3.95 (s, 3H), 5.30 (s, 1H), 5.85 (s) 1H), 7.19 (d, 1H), 7.38 (m, 1H), 7.43 (s, 1H).

LRMS: m/z 267, 269 (M+1)⁺

Preparation 94

### 1-(4-Bromo-2-methylphenyl)-3-(2-hydroxyethoxy)-1H-pyrazole

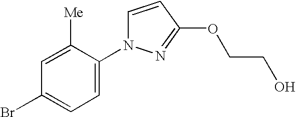

2-Bromoethanol (1.55 ml, 21.8 mmol) was added to a mixture of the alcohol from preparation 92 (2.76 g, 10.9 mmol) and potassium carbonate (3.01 g, 21.8 mmol) in N,N-dimethylformamide (50 ml), and the reaction stirred at 80° C. for 5 hours. The cooled mixture was concentrated in vacuo, the residue suspended in ethyl acetate (250 ml), and the mixture washed with water (5×50 ml). The organic phase was dried (MgSO₄), filtered and evaporated in vacuo. The crude product was purified by column chromatography on silica gel using dichloromethane:ether (80:20) as eluant and crystallised from di-isopropyl ether to give the desired product as buff-coloured crystals, (1.61 g, 50%).

mp 104-105° C.

¹H nmr (CDCl₃, 400 MHz) δ: 2.24 (s, 3H), 2.58 (br, s, 1H), 3.92 (m, 2H), 4.36 (t, 2H), 5.84 (d, 1H), 7.15 (d, 1H), 7.35 (m, 2H), 7.40 (s, 1H).

Anal. Found: C, 48.38; H, 4.30; N, 9.34. C₁₂H₁₃BrN₂O₂ requires C, 48.50; H, 4.41; N, 9.43%.

Preparation 95

### 3-(2-Benzyloxyethoxy)-1-(4-bromo-2-methylphenyl)-1H-pyrazole

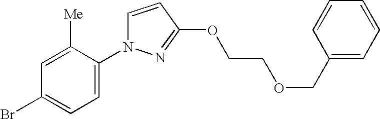

A solution of the alcohol from preparation 94 (1.55 g, 5.2 mmol) in tetrahydrofuran (12 ml) was added to a suspension of sodium hydride (229 mg, 60% dispersion in mineral oil, 5.73 mmol) in tetrahydrofuran (10 ml), and the resulting mixture stirred for 2 minutes under a nitrogen atmosphere. Benzyl bromide (681 μl, 5.73 mmol) was then added and the reaction heated under reflux for 16 hours. The cooled reaction mixture was poured into brine (70 ml) and extracted with ethyl acetate (3×50 ml). The combined organic solutions were dried (MgSO₄), filtered and concentrated in vacuo to give a yellow oil. The crude product was purified by column chromatography on silica gel using an elution gradient of hexane:ethyl acetate (90:10 to 80:20) to give the title compound as a colourless oil, (1.93 g, 96%).

¹H nmr (CDCl₃, 400 MHz) δ: 2.24 (s, 3H), 3.80 (t, 2H), 4.38 (t, 2H), 4.60 (s, 2H), 5.66 (s, 1H), 7.12 (d, 1H), 7.21 (m, 2H), 7.32 (m, 5H), 7.40 (s, 1H).

LRMS: m/z 409, 411 (M+23)⁺

Preparation 96

### 3-Methoxy-1-[(2-methyl4-trimethylstannyl)phenyl]-1H-pyrazole

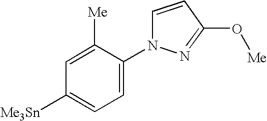

Tetrakis(triphenylphosphine)palladium (0) (30 mg, 0.026 mmol) was added to a solution of the bromide from preparation 93 (659 mg, 2.47 mmol), and hexamethylditin (889 mg, 2.71 mmol) in 1,4-dioxan (8 ml), and nitrogen bubbled through the resulting mixture. The reaction was heated under reflux for 4½ hours, then tlc analysis showed starting material remaining. Additional tetrakis(triphenylphosphine)palladium (0) (48 mg) was added and the reaction heated under reflux for a further 16 hours. 50% Aqueous potassium fluoride solution (5 ml) was added to the cooled reaction, the mixture stirred for 15 minutes, then filtered through Arbocel®, washing through with ether. The filtrate was washed with brine (30 ml), dried (MgSO₄), filtered and evaporated in vacuo. The crude product was purified by column chromatography on silica gel using pentane:ether (90:10) as eluant to give the title compound as a pale yellow oil, (598 mg, 69%).

¹H nmr (CDCl₃, 400 MHz) δ: 0.27 (s, 9H), 2.26 (s 3H), 3.92 (s, 3H), 5.80 (s, 1H), 7.21 (m, 2H), 7.35 (m, 2H).

Preparation 97

### 3-(2-Benzyloxyethoxy)-1-[2-methyl-4-(trimethylstannyl)phenyl]-1H-pyrazole

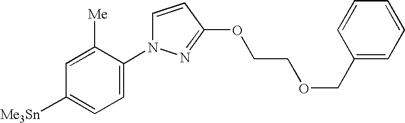

Tetrakis(triphenylphosphine)palladium (0) (286 mg, 0.25 mmol) was added to a solution of the bromide from preparation 95 (1.92 g, 4.96 mmol), and hexamethylditin (1.78 g, 5.45 mmol) in 1,4-dioxan (18 ml), and nitrogen bubbled through the resulting mixture. The reaction was heated under reflux for 2 hours, then cooled. Potassium fluoride solution (5 ml, 50%) was added, the mixture stirred for 30 minutes, and filtered though Arbocel®, washing through well with ethyl acetate (150 ml). The filtrate was washed with brine (2×30 ml), dried (MgSO₄), filtered and evaporated in vacuo. The residue was purified by column chromatography on silica gel using hexane:ether (84:16) to afford the desired product as a crystalline solid, (1.87 g, 80%).

mp 50-52° C.

¹H nmr (CDCl₃, 400 MHz) δ: 0.28 (s, 9H), 2.24 (s, 3H), 3.80 (t, 2H), 4.40 (t, 2H), 4.60 (s, 2H), 5.82 (s, 1H), 7.22 (m, 3H), 7.33 (m, 6H).

Anal. Found: C, 56.21; H. 5.97; N, 5.95. C₂₂H₂₈N₂O₂Sn requires C, 56.08; H, 5.99; N, 5.95%.

Preparation 98

### Methyl 2-{4-[4-(3-methoxy-1H-pyrazol-1-yl}-3-methylphenyl)-1,2,3,6-tetrahydropyridin-1-ylsulphonyl}-2-methyl-propanoate

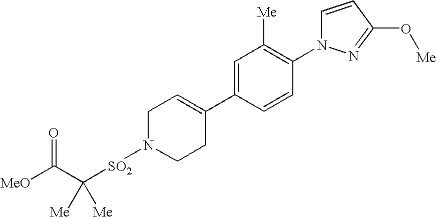

Tris(dibenzylideneacetone)dipalladium(0) (30.7 mg, 0.034 mmol) was added to a solution of the vinyl triflate from preparation 29 (727 mg, 1.84 mmol), the stannane from preparation 96 (590 mg, 1.68 mmol), and triphenylarsine (104 mg, 0.36 mmol) in 1-methyl-2-pyrrolidinone (4 ml), and the solution stirred under a nitrogen atmosphere. Copper (I) iodide (16 mg, 0.17 mmol) was added, the solution de-gassed, and the reaction then stirred at 60° C. for 30 minutes, and at 75° C. for a further 4½ hours. Potassium fluoride solution (3 ml, 50%) was added to the cooled reaction, stirring continued for 15 minutes, and the mixture filtered through Arbocel®, washing through with ethyl acetate (150 ml). The filtrate was washed with water (30 ml), brine (30 ml), dried (MgSO₄), filtered and evaporated in vacuo. The residual orange foam was purified by column chromatography on silica gel using pentane:ether (50:50) to afford the title compound as a pale yellow gum, (588 mg, 81%).

¹H nmr (CDCl₃, 400 MHz) δ: 1.63 (s, 6H), 2.30 (s, 3H), 2.59 (m, 2H), 3.60 (t, 2H), 3.79 (s, 3H), 3.94 (s, 3H), 4.08 (m, 2H), 5.81 (d, 1H), 6.00 (m, 1H), 7.21 (m, 3H), 7.36 (s, 1H).

LRMS: m/z 434 (M+1)⁺

Preparation 99

### Methyl 2-{4-[4-(3-{2-benzyloxyethoxy}-1H-pyrazol-1-yl}-3-methylphenyl]-1,2,3,6-tetrahydropyridin-1-ylsulphonyl}-2-methyl-propanoate

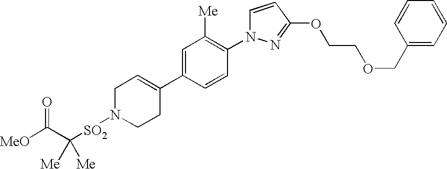

The title compound was obtained as a yellow oil (75%) from the triflate from preparation 29 and the stannane of preparation 97, using a similar method to that described in preparation 98.

¹H nmr (CDCl₃, 400 MHz) δ: 1.64 (s, 6H), 2.27 (s, 3H), 2.58 (m, 2H), 3.59 (m, 2H), 3.78 (s, 3H), 3.80 (t, 2H), 4.09 (m, 2H), 4.39 (t, 2H), 4.60 (s, 2H), 5.85 (s, 1H), 6.00 (m, 1H), 7.21 (m, 4H), 7.34 (m, 5H).

LRMS: m/z 576 (M+23)⁺

Preparation 100

### Methyl 2-{4-[4-(3-methoxy-1H-pyrazol-1-yl}-3-methylphenyl]piperidin-1-ylsulphonyl}-2-methylpropanoate

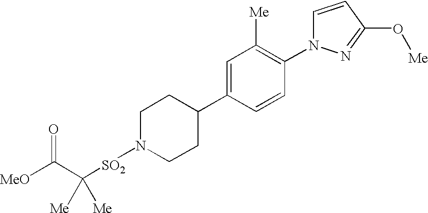

10% Palladium on charcoal (60 mg) was added to a solution of the 1,2,3,6-tetrahydropyridine from preparation 98 (580 mg, 1.38 mmol) in methanol (20 ml), and the mixture hydrogenated at 50 psi and room temperature for 6 hours. Tlc analysis showed starting material remaining, so additional 10% palladium on charcoal (50 mg) was added, and the mixture hydrogenated for a further 18 hours. The reaction mixture was filtered through Arbocel®, the filtrate suspended in dichloromethane (50 ml), re-filtered through Arbocel®, and the filtrate evaporated in vacuo, to give the desired product as a colourless solid, (365 mg, 61%).

mp 109-110° C.

¹H nmr (CDCl₃, 400 MHz) δ: 1.61 (s, 6H), 1.75-1.86 (m, 4H), 2.25 (s, 3H), 2.62 (m, 1H), 3.02 (m, 2H), 3.79 (s, 3H), 3.94 (m, 5H), 5.80 (d, 1H), 7.06 (m, 2H), 7.21 (m, 2H).

LRMS: m/z 458 (M+23)⁺

Preparation 101

### Methyl 2-{4-[4-(3-{2-hydroxyethoxy}-1H-pyrazol-1-yl}-3-methylphenyl]piperidin-1-ylsulphonyl}-2-methylpropanoate

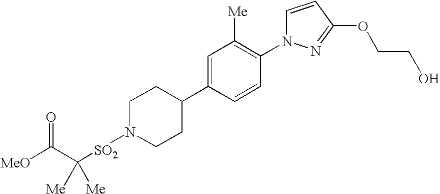

A mixture of the benzyl ether from preparation 99 (790 mg, 1.42 mmol) and 10% palladium on charcoal (160 mg) in ethanol (35 ml) was hydrogenated at 50 psi and room temperature for 17 hours. Tlc analysis showed starting material remaining, so acetic acid (2 ml), and additional 10% palladium on charcoal (80 mg) were added, and the reaction continued for a further 48 hours, with additional 10% palladium on charcoal (160 mg) added portionwise. The reaction mixture was filtered through Arbocel®, washing through with ethanol, and the filtrate concentrated in vacuo. The residue was partitioned between ethyl acetate (100 ml) and saturated sodium bicarbonate solution (100 ml), the layers separated and the organic phase dried (MgSO₄), filtered and evaporated in vacuo to give the title compound as a colourless oil, (630 mg, 95%).

¹H nmr (DMSO-d₆, 400 MHz) δ: 1.46-1.62 (m, 8H), 1.80 (m, 2H), 2.19 (s, 3H), 2.71 (m, 1H), 3.02 (m, 2H), 3.10 (m, 2H), 3.62-3.79 (m, 5H), 4.10 (m, 2H), 4.60 (m, 1H), 5.84 (s, 1H), 7.12 (m, 1H), 7.19 (m, 2H), 7.69 (s, 1H).

LRMS: m/z 488 (M+23)⁺

### Preparation 102

### Methyl 2-methyl-2-{4-[3-methyl-4-(1,3-thiazol-2-yl)phenyl]piperidin-1-ylsulphonyl}-propanoate

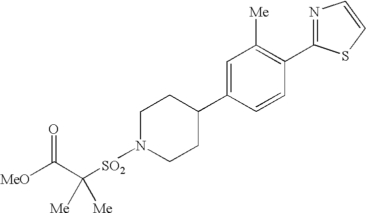

Bis(triphenylphosphine)palladium (II) chloride (49 mg, 0.07 mmol) was added to a solution of the bromide from preparation 26 (577 mg, 1.38 mmol) and 2-(trimethylstannyl)-1,3-thiazole (Synthesis, 1986, 757) (372 mg, 1.5 mmol) in tetrahydrofuran (3.5 ml), and the resulting mixture was de-gassed, and placed under an argon atmosphere. The reaction was heated under reflux for 17 hours. Tlc analysis showed starting material remaining, so additional 2-(trimethylstannyl)-1,3-thiazole (173 mg, 0.8 mmol) and bis(triphenylphosphine)palladium (II) chloride (49 mg, 0.07 mmol) were added, the mixture was de-gassed, and then heated under reflux for a further 17 hours. The cooled mixture was concentrated in vacuo, and the residue purified by column chromatography on silica gel using an elution gradient of hexane:ethyl acetate (91:9 to 66:34). The product was re-purified by column chromatography on silica gel using ether as eluant to give the title compound as a buff-coloured solid, (240 mg, 40%).

mp 111-114° C.

¹H nmr (DMSO-d₆, 400 MHz) δ: 1.52 (s, 6H), 1.58 (m, 2H), 1.81 (m, 2H), 2.45 (s, 3H), 2.74 (m, 1H) 3.04 (m, 2H), 3.74 (m, 5H), 7.18 (d, 1H), 7.21 (s, 1H), 7.62 (d, 1H), 7.78 (d, 1H), 7.92 (d, 1H),

LRMS: m/z 445 (M+23)⁺

Anal. Found: C, 56.64; H, 6.19; N, 6.55. C₂₀H₂₆N₂S₂O₄ requires C, 56.85; H, 6.20; N, 6.63%.

Preparation 103

### 2-{4-[4-(3-Methoxy-1H-pyrazol-1-yl}-3-methylphenyl]piperidin-1-ylsulphonyl}-2-methylpropanoic acid

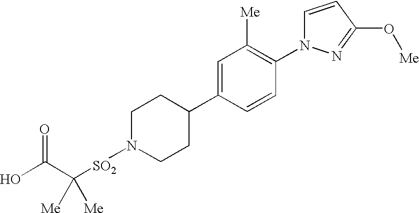

A mixture of the methyl ester from preparation 100 (355 mg, 0.82 mmol), and aqueous sodium hydroxide (5.9 ml, 1M, 5.9 mmol) in methanol (5 ml) and 1,4-dioxan (5 ml) was heated under reflux for 2 hours. The cooled reaction was diluted with water and acidified to pH 3 using hydrochloric acid (2N). The resulting precipitate was filtered off, washed with water, and dried under vacuum at 75° C. to give the title compound as a white powder, (281 mg, 82%).

¹H nmr (CDCl₃, 400 MHz) δ: 1.63 (s, 6H), 1.70-1.90 (m, 4H), 2.24 (s, 3H), 2.62 (m, 1H), 3.04 (m, 2H), 3.90 (s, 3H), 3.98 (m, 2H), 5.80 (s, 1H), 7.04 (m, 3H), 7.32 (m, 1H).

Anal. Found: C, 56.78; H, 6.40; N, 9.71. C₂₀H₂₇N₃O₅S requires C, 56.99; H, 6.46; N, 9.97%

Preparation 104

### 2-{4-[4-(3-{2-Hydroxyethoxy}-1H-pyrazol-1-yl}-3-methylphenyl]piperidin-1-ylsulphonyl}-2-methylpropanoic acid

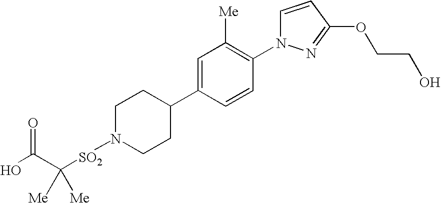

A mixture of the methyl ester from preparation 101(520 mg, 1.2 mmol), and aqueous sodium hydroxide (3.6 ml, 1M, 3.6 mmol) in 1,4-dioxan (5 ml) was heated under reflux for 2½ hours. The cooled reaction was partitioned between water (100 ml) and ethyl acetate (100 ml), acidified to pH 2 using hydrochloric acid (2N), and the phases separated. The aqueous layer was extracted with ethyl acetate (2×35 ml), the combined organic solutions dried (MgSO₄), filtered and concentrated in vacuo. The residue was triturated with ether twice, to afford the title compound as a white solid, (338 mg, 62%).

¹H nmr (DMSO-d₆, 300 MHz) δ: 1.47 (s, 6H), 1.59 (m, 2H), 1.79 (m, 2H), 2.19 (s, 3H), 2.70 (m, 1H), 3.02 (m, 2H), 3.64 (m, 2H), 3.79 (m, 2H), 4.09 (t, 2H), 4.62 (m, 1H), 5.84 (s, 1H), 7.12 (m, 1H), 7.18 (m, 2H), 7.69 (s, 1H), 13.1 (br, s, 1H).

LRMS: m/z 474 (M+23)⁺

Preparation 105

### 2-Methyl-2-{4-[3-methyl-4-(1,3-thiazol-2-yl)phenyl]piperidin-1-ylsulphonyl}-propanoic acid

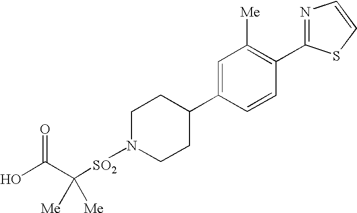

The title compound was obtained as a white solid (92%) from the methyl ester of preparation 102, following a similar procedure to that described in preparation 104.

¹H nmr (DMSO-d₆, 400 MHz) δ: 1.47 (s, 6H), 1.60 (m, 2H), 1.80 (m, 2H), 2.45 (s, 3H), 2.70 (m, 1H), 3.03 (m, 2H), 3.78 (m, 2H), 7.18 (d, 1H), 7.21 (s, 1H), 7.63 (d, 1H), 7.78 (s, 1H), 7.92 (s, 1H), 13.37 (br, s, 1H).

Anal. Found: C, 55.28; H, 5.90; N, 6.70. C₁₉H₂₄N₂O₄S₂ requires C, 55.86; H, 5.92; N, 6.86%.

Preparation 106

### Methyl 1-{[4-(4-bromo-3-methylphenyl)piperidin-1-yl]sulfonyl}-3-cyclopentene-1-carboxylate

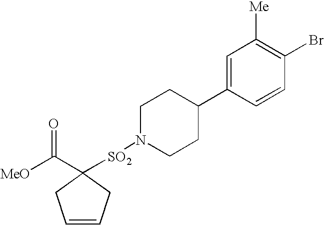

A suspension of sodium hydride (1.1 g, 60% dispersion in mineral oil, 28 mmol) was cooled to 0° C. in anhydrous N-methyl pyrrolidinone (30 ml) under nitrogen. A solution of the ester from preparation 25 (10 g, 26 mmol) in N-methyl pyrrolidinone (70 ml) was added dropwise with stirring and the reaction mixture allowed to warm to ambient temperature over 50 minutes. 1,4-dichlorobut-2-ene (3.0 ml, 28 mmol) and tetrabutylammonium bromide (8.3 g, 26 mmol) were added to the reaction mixture and after a further 3 hours an additional portion of sodium hydride (1.1 g, 60% dispersion in mineral oil, 28 mmol) was added. The mixture was stirred for a further 2 days. The reaction mixture was partitioned between ethyl acetate (300 ml) and water (300 ml) and the layers separated. The aqueous layer was extracted with ethyl acetate (300 ml) and the combined organic extracts were dried (Na₂SO₄), filtered and concentrated in vacuo. The residue was purified by flash chromatography eluting with dichloromethane to give the title compound as a white solid (7.4 g, 65%).

¹H nmr (DMSO-d₆, 400 MHz) δ: 1.45 (m, 2H), 1.75 (m, 2H), 2.28 (s, 3H), 2.64 (m, 1H), 2.95 (m, 4H), 3.14 (d, 2H), 3.75 (s, 3H), 3.78 (s, 2H), 5.63 (s, 2H), 6.98 (d, 1H), 7.21 (s, 1H), 7.43 (d, 1H).

LRMS: m/z 464/466 (M+23)⁺.

Preparation 107

### Methyl(1α,3α,4α)-1-{[4-(4-bromo-3-methylphenyl)piperidin-1-yl]sulfonyl}-3,4-dihydroxycyclopentanecarboxylate

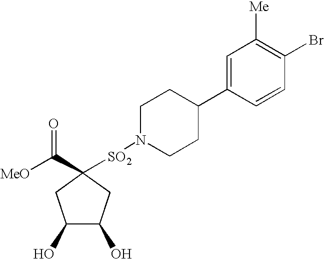

N-methylmorpholine N-oxide (580 mg, 4.97 mmol) and osmium tetroxide (2.5 weight % in tert-butanol, 1.1 ml, 0.136 mmol) were added to a solution of the cyclopentene from preparation 106 (2.0 g, 4.52 mmol) in dioxan (20 ml), water (0.1 ml), and the solution stirred at room temperature for 18 hours. The reaction mixture was partitioned between ethyl acetate (200 ml) and water (300 ml) and the layers separated. The aqueous layer was extracted with ethyl acetate (2×200 ml), and the combined organic extracts were dried (Na₂SO₄), filtered and concentrated in vacuo. The residue was purified by column chromatography on silica gel using dichloromethane/methanol (100:0 to 97:3) as eluant to afford the title compound as a white solid (1.2 g, 56%).

¹H nmr (DMSO-d₆, 400 MHz) δ: 1.47 (m, 2H), 1.77 (m, 2H), 2.28 (m, 5H), 2.42 (s, 2H), 2.63 (m, 1H), 2.91 (m, 2H), 3.75 (m, 5H), 3.85 (s, 2H), 4.62 (s, 2H), 6.98 (d, 1H), 7.21 (s, 1H), 7.43 (d, 1H).

LRMS: m/z 498/500 (M+23)⁺.

Preparation 108

### Methyl(1α,3β,4β)-1-{[4-(4-bromo-3-methylphenyl)piperidin-1-yl]sulfonyl}-3,4-dihydroxycyclopentanecarboxylate

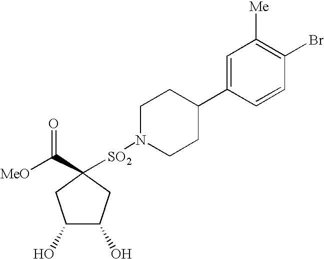

Silver acetate (2.1 g, 12.46 mmol) and iodine (1.5 g, 5.81 mmol) were added to a solution of the cyclopentene from preparation 106 (2.45 g, 5.54 mmol) in glacial acetic acid (125 ml) and the mixture was stirred at ambient temperature for 1 hour. Wet acetic acid (2.5 ml of a 1:25 water/glacial acetic acid mixture) was then added and the reaction was heated to 95° C. for 3 hours and then stirred at ambient temperature for 18 hours. Sodium chloride was added to the mixture and the resulting precipitate was filtered through arbocel® and then washed with toluene. The resulting filtrate was concentrated in vacuo, azeotroped with toluene to give a solid which was triturated with diisopropyl ether. This solid was further purified by flash chromatography eluting with dichloromethane to give the intermediate monoacetate compound as a beige solid (1.35 g, 50%). 1N sodium hydroxide (4 ml) was added to a solution of the monoacetate intermediate in dioxan/methanol (12 ml/8 ml) and the reaction was stirred at ambient temperature for 1 hour. The solvent was removed under reduced pressure, and the residue was partitioned between ethyl acetate (50 ml) and water (75 ml), and the layers separated. The aqueous layer was extracted with ethyl acetate (2×50 ml), and the combined organic extracts were dried (Na₂SO₄), filtered and concentrated in vacuo to give the title compound as a white solid (875 mg, 70%).

¹H nmr (DMSO-d₆, 400 MHz) δ: 1.55 (m, 2H), 1.87 (m, 2H), 2.18 (m, 2H), 2.30 (s, 3H), 2.63 (m, 3H), 2.98 (t, 2H), 3.72 (m, 7H), 4.68 (s, 2H), 6.98 (d, 1H), 7.22 (s, 1H), 7.43 (d, 1H).

LRMS: m/z 498/500(M+23)⁺.

Preparation 109

### Methyl(3aα,5α,6aα)-5-{[4-(4-bromo-3-methylphenyl)piperidin-1-yl]sulfonyl}-2,2-dimethyltetrahydro-3aH-cyclopenta[d][1,3]dioxole-5-carboxylate

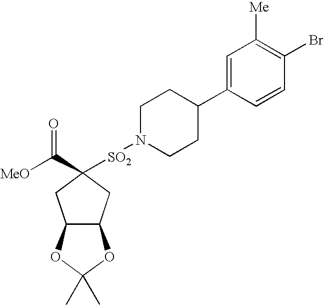

2,2-Dimethoxypropane (0.74 ml, 6 mmol) and p-toluenesulfonic acid (60 mg, 0.3 mmol) were added to a solution of the diol from preparation 107 (1.43 g, 3 mmol) in anhydrous dimethylformamide (10 ml) under nitrogen. The reaction was warmed to 50° C. for 4.5 hours. The mixture was diluted with ethyl acetate (50 ml) and water (40 ml) and the layers separated. The aqueous layer was extracted with ethyl acetate (2×50 ml), and the combined organic extracts were dried (Na₂SO₄), filtered and concentrated in vacuo. The resulting solid was recrystalised from ethyl acetate/di-isopropyl ether to give the title compound as a white solid (1.05 g, 70%).

¹H nmr (DMSO-₆, 400 MHz) δ: 1.17 (s, 3H), 1.20 (s, 3H), 1.47 (m, 2H), 1.77 (m, 2H), 2.23 (m, 2H), 2.32 (s, 3H), 2.65 (m, 3H), 2.95 (t, 2H), 3.72 (m, 5H), 4.64 (s, 2H), 6.98 (d, 1H), 7.21 (s, 1H), 7.43 (d, 1H).

LRMS: m/z 538/540 (M+23)⁺.

Preparation 110

### Methyl(3aβ,5α,6aβ)-5-{[4-(4-bromo-3-methylphenyl)piperidin-1-yl]sulfonyl}-2,2-dimethyltetrahydro-3aH-cyclopenta[d][1,3]dioxole-5-carboxylate

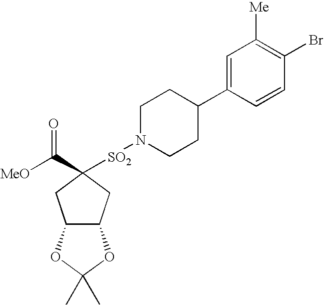

The title compound was prepared from the diol from preparation 108 in a similar procedure to that described in preparation 109. The title compound was isolated as a pale yellow solid (1.3 g, 75%).

¹H nmr (DMSO-d₆, 400 MHz) δ: 1.11 (s, 3H), 1.42 (s, 3H), 1.57 (m, 2H), 1.78 (m, 2H), 2.18 (m, 2H), 2.30 (s, 3H), 2.62 (m, 1H), 2.78 (m, 2H), 2.98 (t, 2H), 3.72 (m, 5H), 4.58 (m, 2H), 6.98 (d, 1H), 7.22 (s, 1H), 7.43 (d, 1H).

LRMS: m/z 538/540(M+23)⁺.

Preparation 111

### Methyl(3aα,5α,6aα)-5-{[4-(4-{6-[2-(tert-butoxy)ethoxy]pyridin-2-yl}-3-methylphenyl)piperidin-1-yl]sulfonyl}-2,2-dimethyltetrahydro-3aH-cyclopenta[d][1,3]dioxole-5-carboxylate

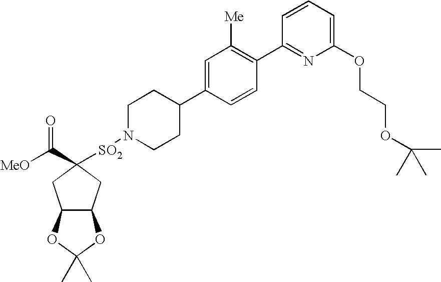

A mixture of the stannane from preparation 127 (2.3 g, 4.78 mmol) and the aryl bromide from preparation 109 (1.9 g, 3.68 mmol), and tetrakis(triphenylphosphine)palladium (0) (213 mg, 0.18 mmol) in toluene (25 ml) was refluxed under nitrogen for 10 hours, then stirred at ambient temperature for 7 hours. The mixture was evaporated in vacuo and to the resulting oil was added ethyl acetate (30 ml) and aqueous potassium fluoride solution (20 ml) and stirred rapidly for 10 minutes. The resulting precipitate was filtered off on arbocel® washing with ethyl acetate. The filtrate was allowed to separate, and the aqueous layer extracted with ethyl acetate (30 ml). The combined organic extracts were dried (Na₂SO₄), filtered and concentrated in vacuo. The residue was purified by column chromatography on silica gel using pentane:ethyl acetate (98:2 to 60:40) as eluant. The resulting solid was recrystalised from ethyl acetate to afford the title compound as a white solid, (1.4 g, 60%).

¹H nmr (DMSO-d₆, 400 MHz) δ: 1.13 (s, 9H), 1.17 (s, 3H), 1.20 (s, 3H), 1.57 (m, 2H), 1.80 (m, 2H), 2.23 (m, 2H), 2.32 (s, 3H), 2.69 (m, 3H), 2.95 (t, 2H), 3.60 (m, 2H), 3.72 (m, 5H), 4.29 (m, 2H), 4.68 (s, 2H), 6.73 (d, 1H), 7.03 (d, 1H) 7.15 (m, 2H), 7.31 (d, 1H), 7.75 (t, 1H).

LRMS: m/z 654 (M+23)⁺.

Preparation 112

### Methyl(3aα,5α,6aα)-5-({4-[4-(6-ethoxypyridin-2-yl)-3-methylphenyl]piperidin-1-yl}sulfonyl)-2,2-dimethyltetrahydro-3aH-cyclopenta[d][1,3]dioxole-5-carboxylate

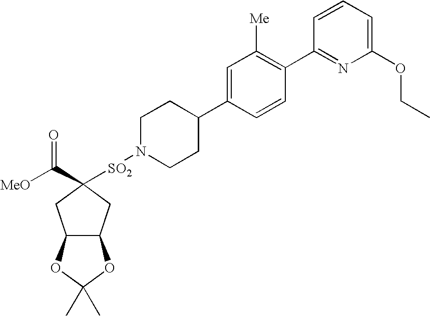

The title compound was prepared from the aryl bromide from preparation 109 and the stannane from preparation 129 in a similar procedure to that described in preparation 111. The title compound was isolated as a white solid (1.1 g, 50%).

¹H nmr (DMSO-d₆, 400 MHz) δ: 1.15 (s, 3H), 1.19 (s, 3H), 1.25 (t, 3H), 1.57 (m, 2H), 1.80 (m, 2H), 2.23 (m, 2H), 2.35 (s, 3H), 2.65 (m, 3H), 2.95 (t, 2H), 3.65 (m, 2H), 3.72 (m, 3H), 4.28 (q, 2H), 4.66 (d, 2H), 6.68 (d, 1H), 7.03 (d, 1H), 7.15 (m, 2H), 7.33 (d, 1H), 7.72 (t, 1H).

LRMS: m/z 581 (M+23)⁺.

Preparation 113

### Methyl(3aβ,5α,6aβ)-5-({4-[4-(6-ethoxypyridin-2-yl)-3-methylphenyl]piperidin-1yl}sulfonyl)-2,2-dimethyltetrahydro-3aH-cyclopenta[d][1,3]dioxole-5-carboxylate

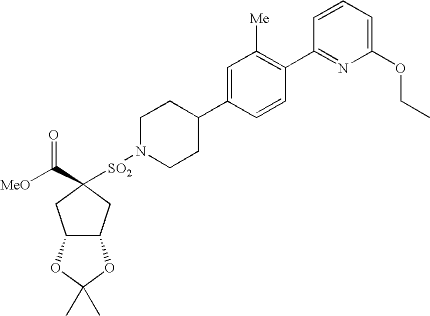

The title compound was prepared from the aryl bromide from preparation 110 and the stannane from preparation 129 in a similar procedure to that described in preparation 111. The title compound was isolated as a white foam (413 mg, 60%).

¹H nmr (DMSO-d₆, 400 MHz) δ: 1.21 (s, 3H), 1.28 (t, 3H), 1.42 (s, 3H), 1.57 (m, 2H), 1.80 (m, 2H), 2.18 (m, 2H), 2.35 (s, 3H), 2.65 (m, 1H), 2.80 (m, 2H), 3.00 (t, 2H), 3.75 (m, 2H), 3.77 (s, 3H), 4.28 (q, 2H), 4.56 (m, 2H), 6.68 (d, 1H), 7.03 (d, 1H), 7.15 (m, 2H), 7.35 (d, 1H), 7.72 (t, 1H).

LRMS: m/z 559 (M+1)⁺.

Preparation 114

### Methyl(3aα,5α,6aα)-5-{4-[4-(3-methoxyphenyl)-3-methylphenyl]piperidin-1-ylsulfonyl}-2,2-dimethyltetrahydro-3aH-cyclopenta[d][1,3]dioxole-5-carboxylate

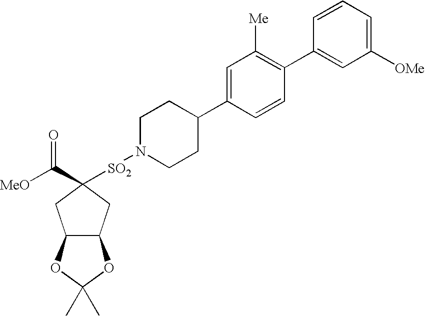

A mixture of the aryl bromide from preparation 109 (1.03, 1.99 mmol), 3-methoxyphenylboronic acid (364 mg, 2.40 mmol), cesium fluoride (606 mg, 4.00mmol), tris(dibenzylideneacetone)dipalladium (0) (91 mg, 0.1 mmol) and tri(o-tolyl)phosphine (61 mg, 0.2 mmol) in 1,2-dimethoxyethane (25 ml) was heated under reflux under nitrogen for 9 hours. The cooled reaction was diluted with water and ethyl acetate, filtered through arbocel®, which was washed with water and ethyl acetate. The organic layer was separated, and washed with brine, dried (Na₂SO₄), filtered and concentrated in vacuo. The residue was purified by column chromatography on silica gel using pentane:ethyl acetate (95:5 to 60:40) as eluant. The title compound was obtained as a white solid (630 mg, 60%).

¹H nmr (DMSO-d₆, 400 MHz) δ: 1.15 (s, 3H), 1.18 (s, 3H), 1.57 (m, 2H), 1.79 (m, 2H), 2.18 (m, 5H), 2.65 (m, 3H), 2.95 (t, 2H), 3.65 (m, 8H), 4.64 (m, 2H), 6.82 (m, 3H), 7.10 (m, 3H), 7.29 (m, 1H).

LRMS: m/z 566 (M+23)⁺.

Preparation 115

### Methyl(3aβ,5α,6aβ)-5-{4-[4-(3-methoxyphenyl)-3-methylphenyl]piperidin-1-ylsulfonyl}-2,2-dimethyltetrahydro-3aH-cyclopenta[d][1,3]dioxole-5-carboxylate

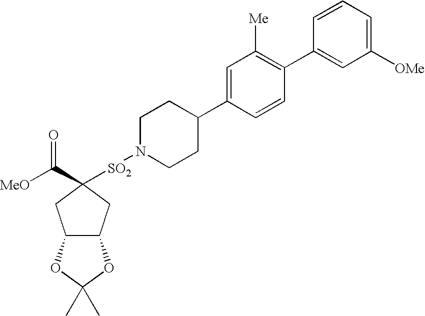

The title compound was prepared from the aryl bromide from preparation 110 in a similar procedure to that described in preparation 114 and was isolated as a white foam (310 mg, 45%).

¹H nmr (DMSO-d₆, 400 MHz) δ: 1.20 (s, 3H), 1.40 (s, 3H), 1.57 (m, 2H), 1.80 (m, 2H), 2.18 (m, 5H), 2.67 (m, 1H), 2.81 (m, 2H), 2.95 (t, 2H), 3.75 (m, 8H), 4.57 (m, 2H), 6.82 (m, 3H), 7.10 (m, 3H), 7.29 (m, 1H).

LRMS: m/z 566 (M+23)⁺.

Preparation 116

### (3aα,5α,6aα)-5-{[4-(4-(6-[2-(tert-butoxy)ethoxy]pyridin-2-yl}-3-methylphenyl)piperidin-1-yl]sulfonyl}-2,2-dimethyltetrahydro-3aH-cyclopenta[d][1,3]dioxole-5-carboxylic acid

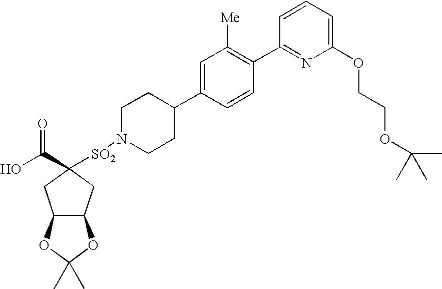

A mixture of the methyl ester from preparation 111 (1.4 g, 2.22 mmol) and aqueous sodium hydroxide (5.5 ml, 2N, 11.1 mmol) in methanol (7 ml) and dioxan (7 ml) was heated under reflux for 1 hour, then allowed to cool. The reaction was concentrated in vacuo, the residue dissolved in water (20 ml), and the solution acidified to pH 4 with glacial acetic acid. The aqueous was extracted with ethyl acetate (2×50 ml) and the collected organic layers dried (Na₂SO₄), filtered and concentrated in vacuo. The resulting oily solid was azeotroped with toluene then triturated with cold ethyl acetate to afford the title compound as a white solid (1.0 g, 75%).

¹H nmr (DMSO-d₆, 400 MHz) δ: 1.13 (s, 9H), 1.16 (s, 3H), 1.28 (s, 3H), 1.57 (m, 2H), 1.75 (m, 2H), 2.26 (m, 5H), 2.59 (m, 3H), 3.05 (t, 2H), 3.60 (m, 2H), 3.72 (d, 2H), 4.28 (m, 2H), 4.58 (m, 2H), 6.73 (d, 1H), 7.03 (d, 1H), 7.15 (m, 2H), 7.31 (d, 1H), 7.75 (t, 1H) 12.9 (s, 1H).

LRMS: m/z 617 (M+1)⁺.

Preparation 117

### (3aα,5α,6aα)-5-({4-[4-(6-ethoxypyridin-2-yl)-3-methylphenyl]piperidin-1-yl}sulfonyl)-2,2-dimethyltetrahydro-3aH-cyclopenta[a][1,3]dioxole-5-carboxylic acid

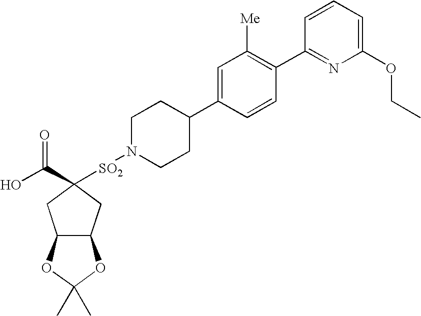

A mixture of the methyl ester from preparation 112 (780 mg, 1.40 mmol) and aqueous sodium hydroxide (3.5 ml, 2N, 6.98 mmol) were dissolved in methanol (5 ml) and dioxan (5 ml) and were heated under reflux for 1.5 hour, then allowed to cool. The reaction was concentrated in vacuo, the residue dissolved in water (20 ml), and the solution acidified to pH 4 with glacial acetic acid. The resulting mixture was extracted with ethyl acetate (2×50 ml) and the collected organic layers dried (Na₂SO₄), filtered and concentrated in vacuo. This afforded the title compound as a white solid (240 mg, 85%).

¹H nmr (DMSO-d₆, 400 MHz) δ: 0.93 (s, 3H), 1.14 (m, 6H), 1.41 (m, 2H), 1.58 (m, 2H), 2.01 (m, 2H), 2.13 (s, 3H), 2.43 (m, 3H), 2.78 (m, 2H), 3.50 (m, 2H), 4.08 (m, 2H), 4.43 (m, 2H), 6.48 (m, 1H), 6.80 (d, 1H), 6.91 (m, 2H), 7.10 (m, 1H), 7.51 (m, 1H) 13.10 (s, 1H).

LRMS: m/z 545 (M+1)⁺.

Preparation 118

### (3aβ,5α,6aβ)-5-({4-[4-(6-ethoxypyridin-2-yl)-3-methylphenyl]piperidin-1-yl}sulfonyl)-2,2-dimethyltetrahydro-3aH-cyclopenta[d][1,3]dioxole-5-carboxylic acid

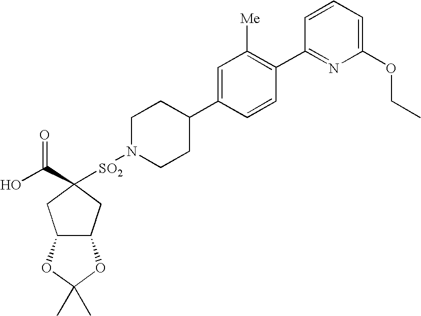

The title compound was prepared from the methyl ester from preparation 113 in a similar procedure to that described in preparation 117 and was isolated as a white foam (250 mg, 65%).

¹H nmr (DMSO-d₆, 400 MHz) δ: 1.21 (s, 3H), 1.28 (t, 3H), 1.42 (s, 3H), 1.61 (m, 2H), 1.80 (d, 2H), 2.18 (m, 2H), 2.35 (s, 3H), 2.65 (m, 1H), 2.80 (m, 2H), 3.00 (t, 2H), 3.78 (d, 2H), 4.28 (q, 2H), 4.56 (m, 2H), 6.68 (d, 1H), 7.01 (d, 1H), 7.15 (m, 2H), 7.35 (d, 1H), 7.72 (t, 1H), 13.65 (s, 1H).

LRMS: m/z 545 (M+1)⁺.

Preparation 119

### (3aα,5α,6aα)-5-{4-[4-(3-methoxyphenyl)-3-methylphenyl]piperidin-1-ylsulfonyl}-2,2-dimethyltetrahydro-3aH-cyclopenta[d][1,3]dioxole-5-carboxylic acid

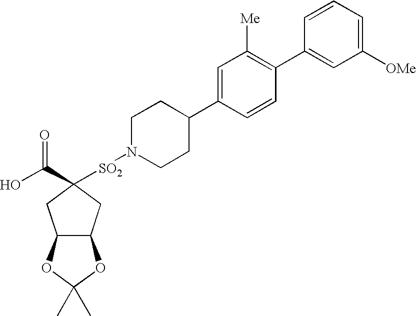

A mixture of the methyl ester from preparation 114 (630 mg, 1.16 mmol) and aqueous sodium hydroxide (3.0 ml, 2N, 5.80 mmol) were dissolved in methanol (5 ml) and dioxan (5 ml) and heated under reflux for 1 hour, then allowed to cool. The reaction was concentrated in vacuo, the residue dissolved in water (20 ml), and the solution acidified to pH 1 with 2N hydrochloric acid. The resulting mixture was extracted with ethyl acetate (2×50 ml) and the collected organic layers dried (Na₂SO₄), filtered and concentrated in vacuo. This afforded the title compound as a white solid (500 mg, 83%).

¹H nmr (DMSO-d₆, 400 MHz) δ: 1.13 (s, 3H), 1.22 (s, 3H), 1.58 (m, 2H), 1.79 (m, 2H), 2.18 (m, 5H), 2.62 (m, 3H), 2.97 (t, 2H), 3.71 (m, 5H), 4.64 (m, 2H), 6.82 (m, 3H), 7.06 (m, 2H), 7.14 (s, 1H), 7.29 (t, 1H).

LRMS: m/z 528 (M−1)⁻.

Preparation 120

### (3aβ,5α,6aβ)-5-{4-[4-(3-methoxyphenyl)-3-methylphenyl]piperidin-1-ylsulfonyl}-2,2-dimethyltetrahydro-3aH-cyclopenta[d][1,3]dioxole-5-carboxylic acid

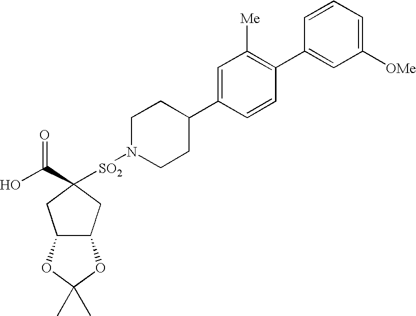

The title compound was prepared from the methyl ester from preparation 115 in a similar procedure to that described in preparation 119 and was isolated as a white foam (250 mg, 85%).

¹H nmr (DMSO-d₆, 400 MHz) δ: 1.20 (s, 3H), 1.40 (s, 3H), 1.58 (m, 2H), 1.80 (m, 2H), 2.15 (m, 2H), 2.18 (s, 3H), 2.65 (m, 1H), 2.78 (m, 2H), 2.99 (t, 2H), 3.77 (m, 5H), 4.56 (m, 2H), 6.82 (m, 3H), 7.10 (m, 3H), 7.29 (t, 1H), 13.78 (s, 1H).

LRMS: m/z 528 (M−1)⁻.

Preparation 121

### (3aα,5α,6aα)-N-hydroxy-5-{[4-(4-{6-[2-(tert-butoxy)ethoxy]pyridin-2-yl}-3-methylphenyl)piperidin-1-yl]sulfonyl}-2,2-dimethyltetrahydro-3aH-cyclopenta[d][1,3]dioxole-5-carboxamide

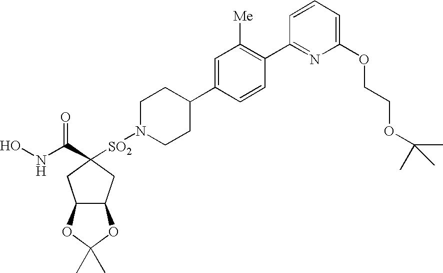

1-(3-Dimethylaminopropyl)-3-ethylcarbodiimide hydrochloride (190 mg, 0.973 mmol) and 1-hydroxy-7-azabenzotriazole (121 mg, 0.892 mmol) were added to a solution of the acid from preparation 116 (500 mg, 0.811 mmol) in N,N-dimethylformamide (6 ml) and pyridine (3 ml) and the reaction was stirred under nitrogen for 50 minutes. Hydroxylamine hydrochloride (170 mg, 2.43 mmol) was then added, and the reaction stirred at room temperature overnight. The reaction was diluted with ethyl acetate (50 ml) and washed with pH 7 phosphate buffer solution (30 ml). The aqueous layer was extracted with ethyl acetate (2×50 ml) and the combined organic extracts were washed with brine, then water, dried (Na₂SO₄), filtered and concentrated in vacuo. The resulting solid was recrystallised from ethyl acetate to afford the title compound as a white solid (260 mg, 50%).

¹H nmr (DMSO-d₆, 400 MHz) δ: 1.15 (s, 9H), 1.16 (s, 3H), 1.20 (s, 3H), 1.59 (m, 2H), 1.75 (m, 2H), 2.17 (m, 2H), 2.31 (s, 3H), 2.59 (m, 1H), 2.66 (d, 2H), 2.99 (t, 2H), 3.59 (m, 2H), 3.64 (d, 2H), 4.28 (m, 2H), 4.62 (m, 2H), 6.72 (d, 1H), 7.03 (d, 1H), 7.15 (m, 2H), 7.29 (d, 1H), 7.70 (t, 1H), 8.85 (s, 1H), 10.82 (s, 1H).

LRMS: m/z 632 (M+1)⁺.

Preparation 122

### (3aα,5α,6aα)-N-hydroxy-5-({4-[4-(6-ethoxypyridin-2-yl)-3-methylphenyl]piperidin-1-yl}sulfonyl)-2,2-dimethyltetrahydro-3aH-cyclopenta[d][1,3]dioxole-5-carboxamide

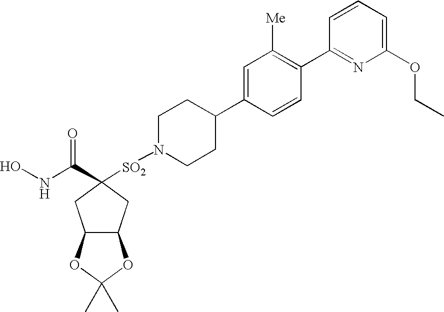

The title compound was prepared from the acid from preparation 117 in a similar procedure to that described in preparation 121, and was isolated as a white solid (150 mg, 60%).

¹H nmr (DMSO-d₆, 400 MHz) δ: 1.13 (s, 3H), 1.21 (s, 3H), 1.25 (t, 3H), 1.61 (m, 2H), 1.76 (m, 2H), 2.18 (m, 2H), 2.32 (s, 3H), 2.60 (m, 1H), 2.77 (d, 2H), 2.99 (t, 2H), 3.63 (d, 2H), 4.25 (q, 2H), 4.63 (m, 2H), 6.68 (d, 1H), 7.02 (d, 1H), 7.14 (m, 2H), 7.30 (d, 1H), 7.71 (t, 1H), 8.86 (s, 1H), 10.82 (s, 1H).

LRMS: m/z 560 (M+1)⁺.

Preparation 123

### (3aβ,5α,6aβ)-N-hydroxy-5-({4-[4-(6-ethoxy-pyridin-2-yl)-3-methylphenyl]piperidin-1-yl}sulfonyl)-2,2-dimethyltetrahydro-3aH-cyclopenta[d][1,3]dioxole-5-carboxamide

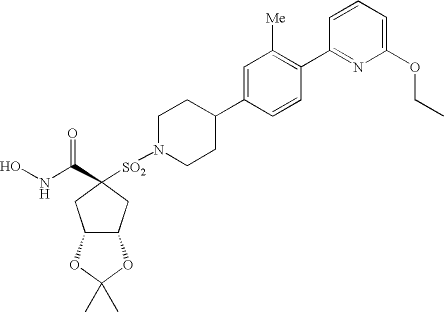

The title compound was prepared from the acid from preparation 118 in a similar procedure to that described in preparation 121. The title compound was isolated after column chromatography (using dichloromethane/methanol 99:1 as eluant) as a white solid (107 mg, 45%).

¹H nmr (DMSO-d₆, 400 MHz) δ: 1.20 (s, 3H), 1.28 (t, 3H), 1.40 (s, 3H), 1.61 (m, 2H), 1.80 (d, 2H), 2.05 (m, 2H), 2.30 (s, 3H), 2.62 (m, 1H), 2.97 (m, 4H), 3.70 (d, 2H), 4.28 (q, 2H), 4.45 (m, 2H), 6.68 (d, 1H), 7.01 (d, 1H), 7.15 (m, 2H), 7.32 (d, 1H), 7.72 (t, 1H), 9.00 (s, 1H), 10.39 (s, 1H).

LRMS: m/z 560 (M+1)⁺.

Preparation 124

### (3aα,5α,6aα)-N-hydroxy-5-{4-[4-(3-methoxyphenyl)-3-methylphenyl]piperidin-1-ylsulfonyl)-2,2-dimethyltetrahydro-3aH-cyclopenta[d][1,3]dioxole-5-carboxamide

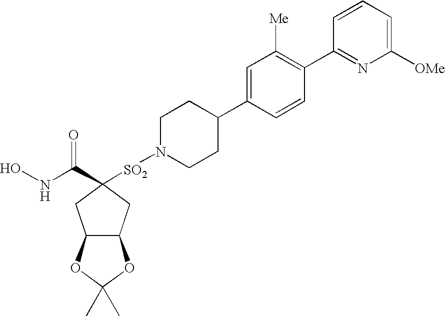

The title compound was prepared from the acid from preparation 119 in a similar procedure to that described in preparation 121, and was isolated as a white solid (110 mg, 43%).

¹H nmr (DMSO-d₆, 400 MHz) δ: 1.13 (s, 3H), 1.22 (s, 3H), 1.58 (m, 2H), 1.77 (m, 2H), 2.18 (m, 5H), 2.58 (m, 1H), 2.75 (d, 2H), 2.98 (t, 2H), 3.65 (d, 2H), 3.75 (s, 3H), 4.63 (m, 2H), 6.82 (m, 3H), 7.08 (s, 2H), 7.15 (s, 1H), 7.28 (t, 1H), 8.85 (s, 1H), 10.82 (s, 1H).

Preparation 125

### (3aβ,5α,6aβ)-N-hydroxy-5-{4-[4-(3-methoxyphenyl)-3-methylphenyl]piperidin-1-ylsulfonyl}-2,2-dimethyltetrahydro-3aH-cyclopenta[d][1,3]dioxole-5-carboxamide

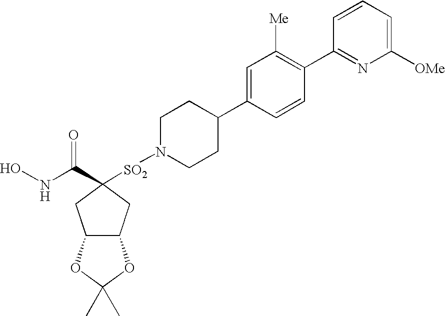

The title compound was prepared from the acid from preparation 120 in a similar procedure to that described in preparation 121. The title compound was isolated after column chromatography (using dichloromethane/methanol 98:2 as eluant) as a white solid (130 mg, 50%).

¹H nmr (DMSO-d₆, 400 MHz) δ: 1.20 (s, 3H), 1.40 (s, 3H), 1.58 (m, 2H), 1.78 (m, 2H), 2.05 (m, 2H), 2.18 (s, 3H), 2.60 (m, 1H), 2.95 (m, 4H), 3.67 (m, 2H), 3.74 (s, 3H), 4.42 (m, 2H), 6.82 (m, 3H), 7.08 (s, 2H), 7.13 (s, 1H), 7.29 (t, 1H), 9.09 (s, 1H), 10.49 (s, 1H).

LRMS: m/z 543 (M−1)⁻.
Preparation 126

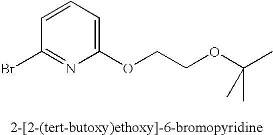

Sodium hydride (6.8 g, 60% dispersion in mineral oil, 0.169 mol) was added portionwise to an ice-cold solution of 2-(tert-butoxy)ethanol (20.0 g, 0.169 mol) in toluene (500 ml) under nitrogen, and the solution stirred for 30 minutes whilst warming to ambient temperature. 2,6-Dibromopyridine (40.0, 0.169 mol) was added, and the reaction heated under reflux for 3 hours. The mixture was allowed to cool to ambient temperature and was diluted with water (1000 ml), and extracted with ethyl acetate (2×400 ml). The combined organic extracts were dried (Na₂SO₄), filtered and evaporated in vacuo to give the title compound as a yellow oil (quantitative).

¹H nmr (CDCl₃, 400 MHz) δ: 1.21 (s, 9H), 3.67 (t, 2H), 4.40 (t, 2H), 6.68 (d, 1H), 7.05 (d, 1H), 7.38 (t, 1H).

LRMS: m/z 296/298 (M+23)⁺.

Preparation 127

### 2-[2-(tert-butoxy)ethoxy]-6-(tributylstannyl)pyridine

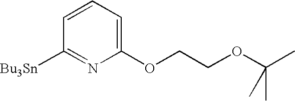

n-Butyllithium (71 ml, 2.5M solution in hexanes, 0.177 mol) was added dropwise to a cooled (−78° C.) solution of the bromide from preparation 126 (46.3 g, 0.169 mol) in anhydrous THF (1000 ml) under nitrogen, so as to maintain the internal temperature <−70° C., and the solution stirred for 10 minutes. Tri-n-butyltin chloride (48 ml, 0.177 mol) was added slowly to maintain the internal temperature <−70° C., and the reaction was then allowed to warm to room temperature over 1 hour. The reaction was diluted with water (1000 ml), the mixture extracted with Et₂O (2×1000 ml), and the combined organic extracts dried (Na₂SO₄), filtered and evaporated in vacuo. The residue was purified by column chromatography on silica gel using pentane:Et₂O (100:1 to 98:2) as eluant, to afford the title compound as a colourless oil, (45.5 g, 55%).

¹H nmr (CDCl₃, 400 MHz) δ: 0.86 (t, 9H), 1.04 (m, 6H), 1.21 (s, 9H), 1.35 (m, 6H), 1.58 (m, 6H), 3.69 (t, 2H), 4.43 (t, 2H), 6.58 (d, 1H), 6.97 (m, 1H), 7.37 (m, 1H).

LRMS: m/z 506/508 (M+23)⁺.

Preparation 128

### 2-bromo-6-ethoxypyridine

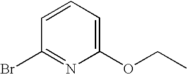

Sodium ethoxide (1.5 g, 63 mmol sodium, in ethanol (30 ml)) was added to 2,6-dibromopyridine (15 g, 63 mmol) in toluene (150 ml) at ambient temperature under nitrogen, and the reaction heated under reflux for 5 hours. The cooled mixture was diluted with water (100 ml), and extracted with ethyl acetate (2×100 ml). The combined organic extracts were dried (Na₂SO₄), filtered and evaporated in vacuo. The residue was purified by column chromatography on silica gel using pentane/ethyl acetate (100:0 to 95:5) as eluant to give the title compound as a yellow oil, (quantitative).

¹H nmr (CDCl₃, 400 MHz) δ: 1.37 (t, 3H), 4.35 (q, 2H), 6.62 (d, 1H), 7.01 (d, 1H), 7.38 (t, 1H).

LRMS: m/z 202/204 (M+1)⁺.

Preparation 129

### 2-ethoxy-6-(tributylstannyl)pyridine

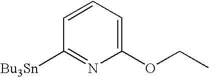

The title compound was prepared from the bromide from preparation 128 in a similar procedure to that described in preparation 127, and was isolated as a colourless oil (1.3 g, 6%).

¹H nmr (CDCl₃, 400 MHz) δ: 0.86 (t, 9H), 1.04 (m, 6H), 1.36 (m, 9H), 1.57 (m, 6H), 4.38 (q, 2H), 6.52 (d, 1H), 6.95 (m, 1H), 7.37 (m, 1H).

LRMS: m/z 434/436 (M+23)⁺.

Preparation 130

### Methyl 4-{(4-(4-bromo-3-methylphenyl)-4-hydroxy-1-piperidin-1-yl]sulfonyl}tetrahydro-2H-pyran-4-carboxylate

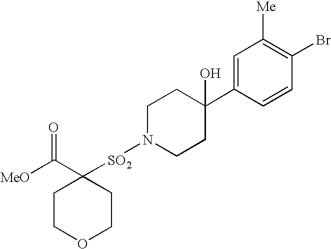

Iso-propylbromide (20 ml, 0.21 mol) was added dropwise over 1 h to a stirred mixture of magnesium (4.7 g, 0.19 mol) in THF (50 ml) and toluene (50 ml), under nitrogen. The mixture was stirred at room temperature for 1 hour and then cooled to 0° C. A solution of 2-bromo-5-iodotoluene (57 g, 0.19 mol) in toluene (50 ml) was added dropwise over 30 min, between 0 and 5° C., and the mixture was stirred at 0° C. for 30 min. The mixture was then added dropwise over 45 min to a stirred suspension the ketone from preparation 16 (50 g, 0.16 mol) in toluene (250 ml), between 0 and 5° C., under nitrogen. The resulting mixture was stirred at 0° C. for 1 hour and then citric acid solution (10%, 400 ml) and ethyl acetate (200 ml) were added. The organic phase was separated and the aqueous phase was re-extracted with ethyl acetate (2×200 ml). The combined organic phases were washed with water (200 ml) and concentrated in vacuo to a solid which was purified by re-crystallisation from toluene (500 ml) to give the title compound as a colourless solid (66 g, 84%).

¹H nmr (CDCl₃, 300 MHz) δ: 1.70-1.77 (m, 2H), 2.02-2.26 (m, 4H), 2.38-2.42 (m, 5H), 3.30 (t, 2H), 3.45 (t, 2H), 3.67-3.75 (m, 2H), 3.88 (s, 3H), 3.99 (dd, 2H), 7.14 (dd, 1H), 7.31 (d, 1H), 7.50 (d, 1H).

Preparation 131

### Methyl 4-{[4-(4-{6-[2-(tert-butoxy)ethoxy]pyridin-2-yl}-3-methylphenyl)piperidin-1-yl]sulfonyl}tetrahydro-2H-pyran-4-carboxylate

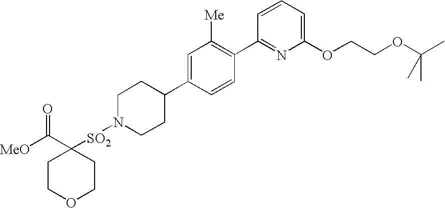

A solution of n-butyllithium in hexanes (2.5M, 3.1 ml, 7.7 mmol) was added dropwise over 5 min to a solution of the bromopyridine from preparation 126 (2.0 g, 7.3 mmol) in THF (20 ml) at −78° C., under nitrogen. The mixture was stirred at −78° C. for 10 min and then tri-iso-propylborate (1.9 ml, 8.0 mmol) was added dropwise over 10 min. The mixture was stirred at −78° C. for 10 min and then allowed to warm to room temperature over 1 hour. The aryl bromide from preparation 27 (2.7 g, 5.8 mmol), palladium acetate (82 mg, 0.36 mmol), triphenylphosphine (191 mg, 0.73 mmol), ethanol (20 ml) and aqueous sodium carbonate (2M, 20 ml) were added and the mixture was heated to reflux for 4 hours, under nitrogen, and then cooled. Ethyl acetate (50 ml) and demineralised water (50 ml) were added and the organic phase was separated. The aqueous phase was re-extracted with ethyl acetate (2×30 ml) and the combined organic phases were washed with demineralised water (50 ml) and then concentrated in vacuo to a solid. Purification by re-crystallisation from methanol (30 ml) gave the title compound as a colourless solid (2.0 g, 60%).

¹H nmr (CD₃OD, 300MHz) δ: 1.12 (s, 9H), 1.50-1.69 (m, 2H), 1.72-1.88 (m, 2H), 1.91-2.05 (m, 2H), 2.24-2.30 (m, 2H), 2.34 (m, 3H), 2.65-2.78 (m, 1H), 3.00-3.23 (m, 4H), 3.61 (t, 2H), 3.70-3.78 (m, 2H), 3.80 (s, 3H), 3.87-3.95 (m, 2H), 4.30 (t, 2H), 6.74 (d, 1H), 7.05 (d, 1H), 7.10-7.17 (m, 2H), 7.33 (d, 1H), 7.73 (t, 1H).

LCMS: m/z 575 (M+H)⁺

Preparation 132

### 4-{[4-4-{6-[2-tert-butoxyethoxy]pyridin-2-yl}-3-methylphenyl)piperidin-1-yl]sulfonyl}-tetrahydro-2H-pyran-4-carboxylic acid

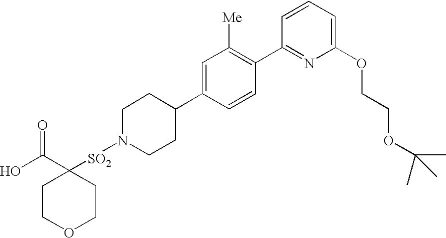

A mixture of the methyl ester from preparation 131 (9.1 g, 16.0 mmol) and aqueous sodium hydroxide (80 ml, 1N, 80.0 mmol) in dioxan (250 ml) were heated under reflux for 2 hours. Methanol (100 ml) and aqueous sodium hydroxide (40 ml, 1N, 40.0 mmol) were added and the mixture refluxed for a further 2 hours, then allowed to cool to ambient temperature. The reaction was concentrated in vacuo, the residue dissolved in water (200 ml), and the solution acidified to pH 4 with glacial acetic acid. The aqueous layer was extracted with ethyl acetate (2×200 ml) and the combined organic extracts were washed with brine (200 ml), then water (2×200 ml), dried (Na₂SO₄), filtered and concentrated in vacuo. The resulting oily solid was azeotroped with toluene then triturated with cold di-isopropyl ether to afford the title compound as a pale yellow solid (7.66 g, 85%).

¹H nmr (DMSO-d₆, 400 MHz) δ: 1.13 (s, 9H), 1.61 (m, 2H), 1.79 (m, 2H), 1.95 (m, 2H), 2.22 (d, 2H), 2.32 (s, 3H), 2.66 (m, 1H), 3.05 (t, 2H), 3.20 (t, 2H), 3.60 (t, 2H), 3.76 (d, 2H), 3.88 (m, 2H), 4.28 (t, 2H), 6.73 (d, 1H), 7.03 (d, 1H), 7.12 (m, 2H), 7.31 (d, 1H), 7.75 (t, 1H), 13.77 (s, 1H).

LRMS: m/z 583 (M+23)⁺.

Preparation 133

### N-Hydroxy-4-[(4-{4-[6-(2-tert-butoxyethoxy)pyridin-2-yl]-3-methylphenyl}piperidin-1-yl)sulfonyl]tetrahydro-2H-pyran-4-carboxamide

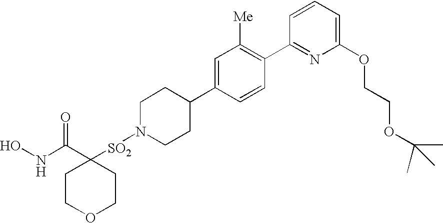

1-(3-Dimethylaminopropyl)-3-ethylcarbodiimide hydrochloride (3.15 g, 16.0 mmol) and 1-hydroxy-7-azabenzotriazole (2.05 g, 15.0 mmol) were added to a solution of the acid from preparation 132 (7.66 g, 14 mmol) in anhydrous dichloromethane (80 ml) and pyridine (80 ml) and the reaction was stirred under nitrogen for 1 hour. Hydroxylamine hydrochloride (2.85 g, 41.0 mmol) was then added, and the reaction stirred at room temperature overnight. The reaction was diluted with dichloromethane (200 ml) and washed with pH 7 phosphate buffer solution (200 ml). The aqueous layer was extracted with dichloromethane (2×200 ml) and the combined organic extracts were washed with dilute aqueous acetic acid (150 ml), brine (150 ml), then water (150 ml), dried (Na₂SO₄), filtered and concentrated in vacuo. The resulting solid was azeotroped with toluene and then recrystallised from ethyl acetate and di-isopropyl ether to afford the title compound as a white solid (6.3 g, 75%).

¹H nmr (DMSO-d₆, 400 MHz) δ: 1.13 (s, 9H), 1.61 (m, 2H), 1.78 (m, 2H), 1.91 (m, 2H), 2.37 (m, 5H), 2.62 (m, 1H), 3.05 (t, 2H), 3.20 (t, 2H), 3.60 (t, 2H), 3.73 (d, 2H), 3.83 (m, 2H), 4.28 (t, 6.73 (d, 1H), 7.03 (d, 1H), 7.12 (m, 2H), 7.31 (d, 1H), 7.72 (t, 1H), 9.05 (s, 1H), 10.90 (s, 1H).

LRMS: m/z 598 (M+23)⁺.

Compound Formulae

These are shown on the following page. 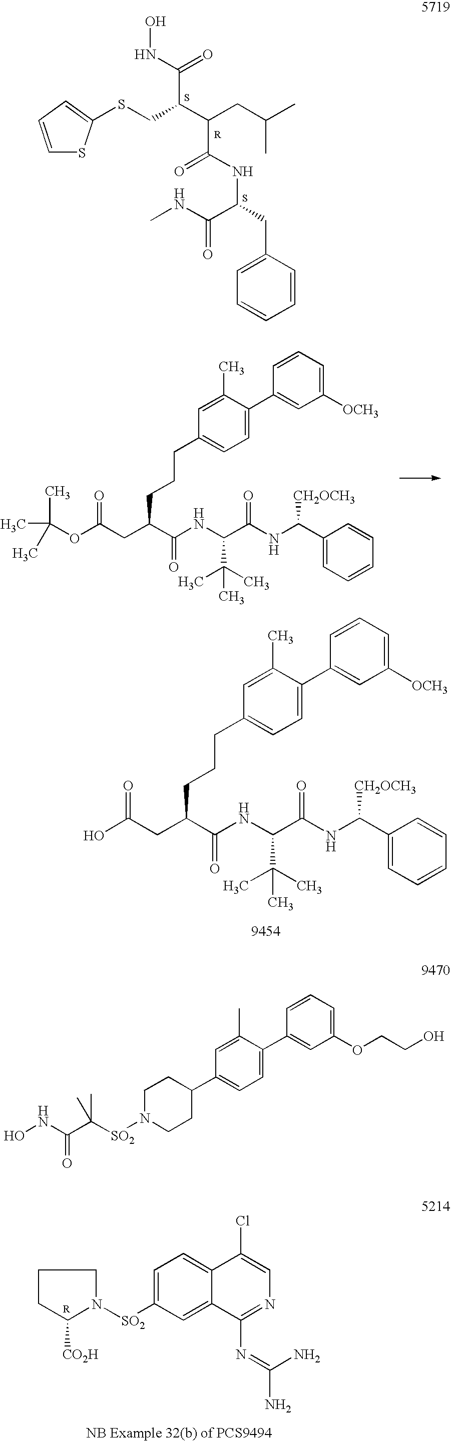
Summary

Combinations of growth factor(s) and/or I:uPA(s) and/or I:MMP(s) are effective at damaged tissue, such as wound, healing.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

References

References for CTGF Section

1. Bradham, D. M.; Igarashi, A.; Potter, R. L.; Grotendorst, G. R.: Connective tissue growth factor: a cysteine-rich mitogen secreted by human vascular endothelial cells is related to the SRC-induced immediate early gene product CEF-10. J. Cell Biol. 114: 1285-1294, 1991.

2. Kim, H.-S.; Nagalla, S. R.; Oh, Y.; Wilson, E.; Roberts, C. T., Jr.; Rosenfeld, R. G.: Identification of a family of low-affinity insulin-like growth factor binding proteins (IGFBPs): characterization of connective tissue growth factor as a member of the IGFBP superfamily. Proc. Nat. Acad. Sci. 94: 12981-12986, 1997.

3. Martinerie, C.; Viegas-Pequignot, E.; Guenard, I.; Dutrillaux, B.; Nguyen, V. C.; Bernheim, A.; Perbal, B.: Physical mapping of human loci homologous to the chicken nov proto-oncogene. Oncogene 7: 2529-2534, 1992.

References for KGF Section

Kelley, M. J.; Pech, M.; Seuanez, H. N.; Rubin, J. S.; O'Brien, S. J.; Aaronson, S. A.: Emergence of the keratinocyte growth factor multigene family during the great ape radiation. Proc. Nat. Acad. Sci. 89: 9287-9291, 1992.

Mattei, M.-G.; deLapeyriere, O.; Bresnick, J.; Dickson, C.; Birnbaum, D.; Mason, I.: Mouse FGF7 (fibroblast growth factor 7) and FGF8 (fibroblast growth factor 8) genes map to chromosomes 2 and 19 respectively. Mammalian Genome 6: 196-197, 1995.

Rubin, J. S.; Osada, H.; Finch, P. W.; Taylor, W. G.; Rudikoff, S.; Aaronson, S. A.: Purification and characterization of a newly identified growth factor specific for epithelial cells. Proc. Nat. Acad. Sci. 86: 802-806, 1989.

Werner, S.; Smola, H.; Liao, X.; Longaker, M. T.; Krieg, T.; Hofschneider, P. H.; Williams, L. T.: The function of KGF in morphogenesis of epithelium and reepithelialization of wounds. Science 266: 819-822, 1994.

Zimonjic, D. B.; Kelley, M. J.; Rubin, J. S.; Aaronson, S. A.; Popescu, N. C.: Fluorescence in situ hybridization analysis of keratinocyte growth factor gene amplification and dispersion in evolution of great apes and humans. Proc. Nat. Acad. Sci. 94: 11461-11465, 1997.

References for TGF Section

1. Bernasconi, P.; Torchiana, E.; Confalonieri, P.; Brugnoni, R.; Barresi, R.; Mora, M.; Conelio, F.; Morandi, L.; Mantegazza, R.: Expression of transforming growth factor-β-1 in dystrophic patient muscles correlates with fibrosis: pathogenetic role of a fibrogenic cytokine. J. Clin. Invest. 96: 1137-1144, 1995.

2. Blanchette, F.; Day, R; Dong, W.; Laprise, M.-H.; Dubois, C. M.: TGF-β-1 regulates gene expression of its own converting enzyme furin. J. Clin. Invest. 99: 1974-1983, 1997.

3. Border, W. A.; Noble, N. A.: Transforming growth factor β in tissue fibrosis. New Eng. J. Med. 331: 1286-1292, 1994.

4. Border, W. A.; Noble, N. A.: Fibrosis linked to TGF-β in yet another disease. (Editorial) J. Clin. Invest. 96: 655-656, 1995.

5. Clouthier, D. E.; Cornerford, S. A.; Hammer, R. E.: Hepatic fibrosis, glomerulosclerosis, and lipodystrophy-like PEPCK-TGF-β-1 transgenic mice. J. Clin. Invest. 100: 2697-2713, 1997.

6. Crawford, S. E.; Stellmach, V.; Murphy-Ullrich, J. E.; Ribeiro, S. M. F.; Lawler, J.; Hynes, R. O.; Boivin, G. P.; Bouck, N.: Thrombospondin-1 is a major activator of TGF-β-1 in vivo. Cell 93: 1159-1170, 1998.

7. Derynck, R.; Jarrett, J. A.; Chen, E. Y.; Eaton, D. H.; Bell, J. R.; Assoian, R. K.; Roberts, A. B.; Sporn, M. B.; Goeddel, D. V.: Human transforming growth factor-5 complementary DNA sequence and expression in normal and transformed cells. Nature 316: 701-705, 1985.

8. Dickinson, M. E.; Kobrin, M. S.; Silan, C. M.; Kingsley, D. M.; Justice, M. J.; Miller, D. A.; Ceci, J. D.; Lock, L. F.; Lee, A.; Buchberg, A. M.; Siracusa, L. D.; Lyons, K. M.; Derynck, R.; Hogan, B. L. M.; Copeland, N. G.; Jenkins, N. A.: Chromosomal localization of seven members of the murine TGF-β superfamily suggests close linkage to several morphogenetic mutant loci. Genomics 6: 505-520, 1990.

9. Dubois, C. M.; Laprise, M.-H.; Blanchette, F.; Gentry, L. E.; Leduc, R.: Processing of transforming growth factor β-1 precursor by human furin convertase. J. Biol. Chem. 270: 10618-10624, 1995.

10. Fujii, D.; Brissenden, J. E.; Derynck, R.; Francke, U.: Transforming growth factor β gene maps to human chromosome 19 long arm and to mouse chromosome 7. Somat. Cell Molec. Genet. 12: 281-288, 1986.

11. Fujii, D. M.; Brissenden, J. E.; Derynck, R.; Francke, U.: Transforming growth factor β gene (TGFβ) maps to human chromosome 19. (Abstract) Cytogenet. Cell Genet. 40: 632, 1985.

12. Grainger, D. J.; Heathcote, K.; Chiano, M.; Snieder, H.; Kemp, P. R.; Metcalfe, J. C.; Carter, N. D.; Spector, T. D.: Genetic control of the circulating concentration of transforming growth factor type β-1. Hum. Molec. Genet. 8: 93-97, 1999.

13. Heldin, C.-H.; Miyazono, K.; ten Dijke, P.: TGF-β signalling from cell membrane to nucleus through SMAD proteins. Nature 390: 465-471, 1997.

14. Marquardt, H.; Lioubin, M. N.; Ikeda, T.: Complete amino acid sequence of human transforming growth factor type β-2. J. Biol. Chem. 262: 12127-12131, 1987.

15. Roberts, A. B.; Sporn, M. B.; Assoian, R. K.; Smith, J. M.; Roche, N. S.; Wakefield, L. M.; Heine, U. I.; Liotta, L. A.; Falanga, V.; Kehrl, J. H.; Fauci, A. S.: Transforming growth factor type β: rapid induction of fibrosis and angiogenesis in vivo and stimulation of collagen formation in vitro. Proc. Nat. Acad. Sci. 83: 4167-4171, 1986.

16. Shenkar, R.; Coulson, W. F.; Abraham, E.: Hemorrhage and resuscitation induce alterations in cytokine expression and the development of acute lung injury. Am. J. Resp. Cell. Mol. Biol. 10: 290-297, 1994.

17. Sporn, M. B.; Roberts, A. B.; Wakefield, L. M.; Assoian, R. K.: Transforming growth factors-β: biological function and chemical structure. Science 233: 532-534, 1986.

18. Stroschein, S. L.; Wang, W.; Zhou, S.; Zhou, Q.; Luo, K.: Negative feedback regulation of TGF-β signaling by the SnoN oncoprotein. Science 286: 771-774, 1999.

References for CSF Section

1. Cantrell, M. A.; Anderson, D.; Cerretti, D. P.; Price, V.; McKereghan, K.; Tushinski, R. J.; Mochizuki, D. Y.; Larsen, A.; Grabstein, K.; Gillis, S.; Cosman, D.: Cloning, sequence, and expression of a human granulocyte/macrophage colony-stimulating factor. Proc. Nat. Acad. Sci. 82: 6250-6254, 1985.

2. Frolova, E. I.; Dolganov, G. M.; Mazo, I. A.; Smirnov, D. V.; Copeland, P.; Stewart, C.; O'Brien, S. J.; Dean, M.: Linkage mapping of the human CSF2 and IL3 genes. Proc. Nat. Acad. Sci. 88: 4821-4824, 1991.

3. Grabstein, K. H.; Urdal, D. L.; Tushinski, R. J.; Mochizuki, D. Y.; Price, V. L.; Cantrell, M. A.; Gillis, S.; Conlon, P. J.: Induction of macrophage tumoricidal activity by granulocyte-macrophage colony-stimulating factor. Science 232: 506-508, 1986.

4. Huebner, K.; Isobe, M.; Croce, C. M.; Golde, D. W.; Kaufman, S. E.; Gasson, J. C.: The human gene encoding GM-CSF is at 5q21-q32, the chromosome region deleted in the 5q-anomaly. Science 230: 1282-1285, 1985.

5. Le Beau, M. M.; Espinosa, R., III; Neuman, W. L.; Stock, W.; Roulston, D.; Larson, R. A.; Keinanen, M.; Westbrook, C. A.: Cytogenetic and molecular delineation of the smallest commonly deleted region of chromosome 5 in malignant myeloid diseases. Proc. Nat. Acad. Sci. 90: 5484-5488, 1993.

6. Le Beau, M. M.; Westbrook, C. A.; Diaz, M. O.; Larson, R. A.; Rowley, J. D.; Gasson, J. C.; Golde, D. W.; Sherr, C. J.: Evidence for the involvement of GM-CSF and FMS in the deletion (5q) in myeloid disorders. Science 231: 984-987, 1986.

7. LeVine, A. M.; Reed, J. A.; Kurak, K. E.; Cianciolo, E.; Whitsett, J. A.: GM-CSF-deficient mice are susceptible to pulmonary group B streptococcal infection. J. Clin. Invest. 103: 563-569, 1999.

8. Metcalf, D.: The molecular biology and functions of the granulocyte-macrophage colony-stimulating factors. Blood 67: 257-267, 1986.

9. Pettenati, M. J.; Le Beau, M. M.; Lemons, R. S.; Shima, E. A.; Kawasaki, E. S.; Larson, R. A.; Sherr, C. J.; Diaz, M. O.; Rowley, J. D.: Assignment of CSF-1 to 5q33.1: evidence for clustering of genes regulating hematopoiesis and for their involvement in the deletion of the long arm of chromosome 5 in myeloid disorders. Proc. Nat. Acad. Sci. 84: 2970-2974, 1987.

10. Sieff, C. A.; Emerson, S. G.; Donahue, R. E.; Nathan, D. G.; Wang, E. A.; Wong, G. G.; Clark, S. C.: Human recombinant granulocyte-macrophage colony-stimulating factor: a multilineage hematopoietin. Science 230: 1171-1173, 1985.

11. Thangavelu, M.; Neuman, W. L.; Espinosa R., III; Nakamura, Y.; Westbrook, C. A.; Le Beau, M. M.: A physical and genetic linkage map of the distal long arm of human chromosome 5. Cytogenet. Cell Genet. 59: 27-30, 1992.

12. Wong, G. G.; Witek, J. S.; Temple, P. A.; Wilkens, K. M.; Leary, A. C.; Luxenberg, D. P.; Jones, S. S.; Brown, E. L.; Kay, R. M.; Orr, E. C.; Shoemaker, C.; Golde, D. W.; Kaufman, R. J.; Hewick, R. M.; Wang, E. A.; Clark, S. C.: Human GM-CSF: molecular cloning of the complementary DNA and purification of the natural and recombinant proteins. Science 228: 810-815, 1985.

13. Yang, Y.-C.; Kovacic, S.; Kriz, R.; Wolf, S.; Clark, S. C.; Wellems, T. E.; Nienhuis, A.; Epstein, N.: The human genes for GM-CSF and IL3 are closely linked in tandem on chromosome 5. Blood 71: 958-961, 1988.

References for EGF Section

1. Brissenden, J. E.; Ullrich, A.; Francke, U.: Chromosomal mapping of loci for insulin-like growth factors I and II and for epidermal growth factor in man. Am. J. Hum. Genet. 36: 133S only, 1984.

2. Brissenden, J. E.; Ullrich, A.; Francke, U.: Human chromosomal mapping of genes for insulin-like growth factors I and II and epidermal growth factor. Nature 310: 781-784, 1984.

3. Carpenter, G.; Cohen, S.: Epidermal growth factor. Ann. Rev. Biochem. 48: 193-216, 1979.

4. Cohen, S.: Isolation of a mouse submaxillary gland protein accelerating incisor eruption and eyelid opening in the new-born animal. J. Biol. Chem. 237: 1555-1562, 1962.

5. Gray, A.; Dull, T. J.; Ullrich, A.: Nucleotide sequence of epidermal growth factor cDNA predicts a 128,000-molecular weight protein precursor. Nature 303: 722-725, 1983.

6. Morton, C. C.; Byers, M. G.; Nakai, H.; Bell, G. I.; Shows, T. B.: Human genes for insulin-like growth factors I and II and epidermal growth factor are located on 12q22-q24.1, 11p15, and 4q25-q27, respectively. Cytogenet. Cell Genet. 41: 245-249, 1986.

7. Sassone-Corsi, P.; Mizzen, C. A.; Cheung, P.; Crosjo, C.; Monaco, L.; Jacquot, S.; Hanauer, A.; Allis, C. D.: Requirement of Rsk-2 for epidermal growth factor-activated phosphorylation of histone H3. Science 285: 886-891, 1999.

9. Smith, J.; Cook, E.; Fotheringham, I.; Pheby, S.; Derbyshire, R.; Eaton, M. A. W.; Doel, M.; Lilley, D. M. J.; Pardon, J. F.; Patel, T.; Lewis, H.; Bell, L. D.: Chemical synthesis and cloning of a gene for human β-urogastrone. Nucleic Acids Res. 10: 4467-4482, 1982.

10. Sudhof, T. C.; Russell, D. W.; Goldstein, J. L.; Brown, M. S.; Sanchez-Pescador, R; Bell, G. I.: Cassette of eight exons shared by genes for LDL receptor and EGF precursor. Science 228: 893-895, 1985.

11. Tsutsumi, O.; Kurachi, H.; Oka, T.: A physiological role of epidermal growth factor in male reproductive function. Science 233: 975-977, 1986.

12. Urdea, M. S.; Merryweather, J. P.; Mullenbach, G. T.; Coit, D.; Heberlein, U.; Valenzuela, P.; Barr, P. J.: Chemical synthesis of a gene for human epidermal growth factor urogastrone and its expression in yeast. Proc. Nat. Acad. Sci. 80: 7461-7465, 1983.

13. Zabel, B. U.; Eddy, R. L.; Lalley, P. A.; Scott, J.; Bell, G. I.; Shows, T. B.: Chromosomal locations of the human and mouse genes for precursors of epidermal growth factor and the β subunit of nerve growth factor. Proc. Nat. Acad. Sci. 82: 469-473, 1985.

References for VEGF Section

1. Carmellet, P.; Ferreira, V.; Breler, G.; Pollefeyt, S.; Kleckens, L.; Gertsenstein, M.; Fahrig, M.; Vandenhoeck, A.; Harpal, K.; Eberhardt, C.; Declercq, C.; Pawling, J.; Moons, L.; Collen, D.; Risau, W.; Nagy, A.: Abnormal blood vessel development and lethality in embryos lacking a single VEGF allele. Nature 380: 435-439, 1996.

2. Ferrara, N.; Carver-Moore, K.; Chen, H.; Dowd, M.; Lu, L.; O'Shea, K. S.; Powell-Braxton, L.; Hillan, K. J.; Moore, M. W.: Heterozygous embryonic lethality induced by targeted inactivation of the VEGF gene. Nature 380: 439-442, 1996.

3. Folkman, J.: Angiogenesis in cancer, vascular, rheumatoid and other disease. Nature Med. 1: 27-31, 1995.

4. Fukumura, D.; Xavier, R.; Sugiura, T.; Chen, Y.; Park, E.-C.; Lu, N.; Selig, M.; Nielsen, G.; Taksir, T.; Jain, R. K.; Seed, B.: Tumor induction of VEGF promoter activity in stromal cells Cell 94: 715-725, 1998.

5. Gerber, H.-P.; Vu, T. H.; Ryan, A. M.; Kowalski, J.; Werb, Z.; Ferrara, N.: VEGF couples hypertrophic cartilage remodeling, ossification and angiogenesis during endochondral bone formation. Nature Med. 5: 623-628, 1999.

6. Holash, J.; Maisonpierre, P. C.; Compton, D.; Boland, P.; Alexander, C. R.; Zagzag, D.; Yancopoulos, G. D.; Wiegand, S. J.: Vessel cooption, regression, and growth in tumors mediated by angiopoietins and VEGF. Science 284: 1994-1998, 1999.

7. Mattei, M.-G.; Borg, J.-P.; Rosnet, O.; Marme, D.; Birnbaum, D.: Assignment of vascular endothelial growth factor (VEGF) and placenta growth factor (PIGF) genes to human chromosome 6p12-p21 and 14q24-q31 regions, respectively. Genomics 32: 168-169, 1996.

8. Millauer, B.; Shawver, L. K.; Plate, K. H.; Risau, W.; Ullrich, A.: Glioblastoma growth inhibited in vivo by a dominant-negative Flk-1 mutant. Nature 367: 576-579, 1994.

9. Soker, S.; Takashima, S.; Miao, H. Q.; Neufeld, G.; Klagsbrun, M.: Neuropilin-1 is expressed by endothelial and tumor cells as an isoform-specific receptor for vascular endothelial growth factor. Cell 92: 735-745, 1998.

10. Springer, M. L.; Chen, A. S.; Kraft, P. E.; Bednarski, M.; Blau, H. M.: VEGF gene delivery to muscle: potential role for vasculogenesis in adults. Molec. Cell 2: 549-558, 1998.

11. Tischer, E.; Mitchell, R.; Hartman, T.; Silva, M.; Gospodarowicz, D.; Fiddes, J. C.; Abraham, J. A.: The human gene for vascular endothelial growth factor: multiple protein forms are encoded through alternative exon splicing. J. Biol. Chem. 266: 11947-11954, 1991.

12. Wei, M.-H.; Popescu, N. C.; Lerman, M. I.; Merrill, M. J.; Zimonjic, D. B.: Localization of the human vascular endothelial growth factor gene, VEGF, at chromosome 6p12. Hum. Genet. 97: 794-797, 1996.

References for Urokinase Section

1. Lijnen, H. R.; Van Hoef, B.; Nelles, L.; Holmes, W. E.; Collen, D.: Enzymatic properties of single-chain and two-chain forms of a lys(158)-to-glu(158) mutant of urokinase-type plasminogen activator. Europ. J. Biochem. 172: 185-188, 1988.

2. Nagai, M.; Hiramatsu, R.; Kaneda, T.; Hayasuke, N.; Arimura, H.; Nishida, M.; Suyama, T.: Molecular cloning of cDNA coding for human preprourokinase. Gene 36: 183-188, 1985.

3. Nelles, L.; Lijnen, H. R.; Collen, D.; Holmes, W. E.: Characterization of recombinant human single chain urokinase-type plasminogen activator mutants produced by site-specific mutagenesis of lysine 158. J. Biol. Chem. 262: 5682-5689, 1987.

4. Rajput, B.; Degen, S. F.; Reich, E.; Waller, E. K.; Axelrod, J.; Eddy, R. L.; Shows, T. B.: Chromosomal locations of human tissue plasminogen activator and urokinase genes. Science 230: 672-674, 1985.

5. Rajput, B.; Marshall, A.; Killary, A. M.; Lalley, P. A.; Naylor, S. L.; Belin, D.; Rickles, R. J.; Strickland, S.: Chromosomal assignments of genes for tissue plasminogen activator and urokinase in mouse. Somat. Cell Molec. Genet. 13: 581-586, 1987.

6. Riccio, A.; Grimaldi, G.; Verde, P.; Sebastio, G.; Boast, S.; Blasi, F.: The human urokinase-plasminogen activator gene and its promoter. Nucleic Acids Res. 13: 1759-2771, 1985.

7. Salerno, G.; Verde, P.; Nolli, M. L.; Corti, A.; Szots, H.; Meo, T.; Johnson, J.; Bullock, S.; Cassani, G.; Blasi, F.: Monoclonal antibodies to human urokinase identify the single-chain pro-urokinase precursor. Proc. Nat. Acad. Sci. 81: 110-114, 1984.

8. Tripputi, P.; Blasi, F.; Verde, P.; Cannizzaro, L. A.; Emanuel, B. S.; Croce, C. M.: Human urokinase gene is located on the long arm of chromosome 10. Proc. Nat. Acad. Sci. 82: 4448-4452, 1985.

References for MMP1 Section

1. Bauer, E. A.; Silverman, N.; Busiek, D. F.; Kronberger, A.; Deuel, T. F.: Diminished response of Werner's syndrome fibroblasts to growth factors PDGF and FGF. Science 234: 1240-1243, 1986.

2. Brinckerhoff, C. E.; Ruby, P. L.; Austin, S. D.; Fini, M. E.; White, H. D.: Molecular cloning of human synovial cell collagenase and selection of a single gene from genomic DNA. J. Clin. Invest. 79: 542-546, 1987.

3. Church, R. L.; Bauer, E. A.; Eisen, A. Z.: Human skin collagenase: assignment of the structural gene to chromosome 11 in both normal and recessive dystrophic epidermolysis bullosa cells using human-mouse somatic cell hybrids. Collagen Rel. Res. 3: 115-124, 1983.

4. Gerhard, D. S.; Jones, C.; Bauer, E. A.; Eisen, A. Z.; Goldberg, G. I.: Human collagenase gene is localized to 11q. (Abstract) Cytogenet. Cell Genet. 46: 619 only, 1987.

5. Goldberg, G. I.; Wilhelm, S. M.; Kronberger, A.; Bauer, E. A.; Grant, G. A.; Eisen, A. Z.: Human fibroblast collagenase: complete primary structure and homology to an oncogene transformation-induced rat protein. J. Biol. Chem. 261: 6600-6605, 1986.

6. Nagase, H.; Barrett, A. J.; Woessner, J. F., Jr.: Nomenclature and glossary of the matrix metalloproteinases. Matrix Suppl. 1: 421-424, 1992.

7. Pendas, A. M.; Santamaria, I.; Alvarez, M. V.; Pritchard, M.; Lopez-Otin, C.: Fine physical mapping of the human matrix metalloproteinase genes clustered on chromosome 11q22.3. Genomics 37: 266-269, 1996.

References for MMP2 Section

1. Becker-Follmann, J.; Gaa, A.; Bausch, E.; Natt, E.; Scherer, G.; von Deimling, O.: High-resolution mapping of a linkage group on mouse chromosome 8 conserved on human chromosome 16Q. Mammalian Genome 8: 172-177, 1997.

2. Brooks, P. C.; Silletti, S.; von Schalscha, T. L.; Friedlander, M.; Cheresh, D. A.: Disruption of angiogenesis by PEX, a noncatalytic metalloproteinase fragment with integrin binding activity. Cell 92: 391-400, 1998.

3. Chen, L. Z.; Harris, P. C.; Apostolou, S.; Baker, E.; Holman, K.; Lane, S. A.; Nancarrow, J. K.; Whitmore, S. A.; Stallings, R. L.; Hildebrand, C. E.; Richards, R. I.; Sutherland, G. R.; Callen, D. F.: A refined physical map of the long arm of human chromosome 16. Genomics 10: 308-312, 1991.

4. Collier, I. E.; Bruns, G. A. P.; Goldberg, G. I.; Gerhard, D. S.: On the structure and chromosome location of the 72- and 92-kDa human type IV collagenase genes. Genomics 9: 429-434, 1991.

5. Devarajan, P.; Johnston, J. J.; Ginsberg, S. S.; Van Wart, H. E.; Berliner, N.: Structure and expression of neutrophil gelatinase cDNA: identity with type IV collagenase from HT1080 cells. J. Biol. Chem. 267: 25228-25232, 1992.

6. Fan, Y.-S.; Eddy, R. L.; Huhtala, P.; Byers, M. G.; Haley, L. L.; Henry, W. M.; Tryggvason, K.; Shows, T. B.: Collagenase type IV (CLG4) is mapped to human chromosome 16q21. (Abstract) Cytogenet. Cell Genet. 51: 996, 1989.

7. Huhtala, P.; Chow, L. T.; Tryggvason, K.: Structure of the human type IV collagenase gene. J. Biol. Chem. 265: 11077-11082, 1990.

8. Huhtala, P.; Eddy, R. L.; Fan, Y. S.; Byers, M. G.; Shows, T. B.; Tryggvason, K.: Completion of the primary structure of the human type IV collagenase preproenzyme and assignment of the gene (CLG4) to the q21 region of chromosome 16. Genomics 6: 554-559, 1990.

9. Irwin, J. C.; Kirk, D.; Gwatkin, R. B. L.; Navre, M.; Cannon, P.; Giudice, L. C.: Human endometrial matrix metalloproteinase-2, a putative menstrual proteinase: hormonal regulation in cultured stromal cells and messenger RNA expression during the menstrual cycle. J. Clin. Invest. 97: 438-447, 1996.

10. Morgunova, E.; Tuuttila, A.; Bergmann, U.; Isupov, M.; Lindqvist, Y.; Schneider, G.; Tryggvason, K.: Structure of human pro-matrix metalloproteinase-2: activation mechanism revealed. Science 284: 1667-1670, 1999.

11. Nagase, H.; Barrett, A. J.; Woessner, J. F., Jr.: Nomenclature and glossary of the matrix metalloproteinases. Matrix Suppl. 1: 421-424, 1992.

References for MMP3 Section

1. Formstone, C. J.; Byrd, P. J.; Ambrose, H. J.; Riley, J. H.; Hernandez, D.; McConville, C. M.; Taylor, A. M. R.: The order and orientation of a cluster of metalloproteinase genes, stromelysin 2, collagenase, and stromelysin, together with D11S385, on chromosome 11q22-q23. Genomics 16: 289-291, 1993.

2. Gatti, R. A.; Sanal, O.; Wei, S.; Charmley, P.; Concannon, P.; Foroud, T.; Reynolds, J.; Lange, K.: Fine mapping the ataxia-telangiectasia locus within the chromosome 11q22-23 region. (Abstract) Am. J. Hum. Genet. 45: A140 only, 1989.

3. Imai, K.; Yokohama, Y.; Nakanishi, I.; et al.: Matrix metalloproteinase 7 (matrilysin) from human rectal carcinoma cells: activation of the precursor, interaction with other matrix metalloproteinases and enzymic properties. J. Biol. Chem. 270: 6691-6697, 1995.

4. Kerr, L. D.; Holt, J. T.; Matrisian, L. M.: Growth factors regulate transin gene expression by c-fos-dependent and c-fos-independent pathways. Science 242: 1424-1427, 1988.

5. Koklitis, P. A.; Murphy, G.; Sutton, C.; Angal, S.: Purification of recombinant human prostromelysin: studies on heat activation to give high-Mr and low-Mr active forms, and a comparison of recombinant with natural stromelysin activities. Biochem. J. 276: 217-221, 1991.

6. Lu, P. C.-S.; Ye, H.; Maeda, M.; Azar, D. T.: Immunolocalization and gene expression of matrilysin during corneal wound healing. Invest. Ophthal. Vis. Sci. 40: 20-27, 1999.

7. Matthews, B. W.; Weaver, L. H.; Kester, W. R.: The conformation of thermolysin. J. Biol. Chem. 249: 8030-8044, 1974.

8. Pendas, A. M.; Santamaria. I.; Alvarez, M. V.; Pritchard, M.; Lopez-Otin, C.: Fine physical mapping of the human matrix metalloproteinase genes clustered on chromosome 11q22.3. Genomics 37: 266-269, 1996.

9. Quinones, S.; Saus, J.; Otani, Y.; Harris, E. D., Jr.; Kurkinen, M.: Transcriptional regulation of human stromelysin. J. Biol. Chem. 264: 8339-8344, 1989.

10. Richardson, P. D.; Davies, M. J.; Born, G. V. R.: Influence of plaque configuration and stress distribution on fissuring of coronary atherosclerotic plaques. Lancet 2: 941-944, 1989.

11. Saarialho-Kere, U. K.; Chang, E. S.; Welgus, H. G.; Parks, W. C.: Distinct localization of collagenase and tissue inhibitor of metalloproteinases: expression in wound healing associated with ulcerative pyogenic granuloma. J. Clin. Invest. 90: 1952-1957, 1992.

12. Saarialho-Kere, U. K.; Pentland, A. P.; Birkedal-Hansen, H.; Parks, W. C.; Welgus, H. G.: Distinct populations of basal keratinocytes express stromelysin-1 and stromelysin-2 in chronic wounds. J. Clin. Invest. 94: 79-88, 1994.

13. Saus, J.; Quinones, S.; Otani, Y.; Nagase, H.; Harris, E. D., Jr.; Kurkinen, M.: The complete primary structure of human matrix metalloproteinase-3: identity with stromelysin. J. Biol. Chem. 263: 6742-6745, 1988.

14. Sellers, A.; Murphy, G.: Collagenolytic enzymes and their naturally occurring inhibitors. Int. Rev. Connect. Tissue Res. 9: 151-190, 1981.

15. Spurr, N. K.; Gough, A. C.; Gosden, J.; Rout, D.; Porteous, D. J.; van Heyningen, V.; Docherty, A. J. P.: Restriction fragment length polymorphism analysis and assignment of the metalloproteinases stromelysin and collagenase to the long arm of chromosome 11. Genomics 2: 119-127, 1988.

16. Sternlicht, M. D.; Lochter, A.; Sympson, C. J.; Huey, B.; Rougier, J.-P.; Gray, J. W.; Pinkel, D.; Bissell, M. J.; Werb, Z.: The stromal proteinase MMP3/stromelysin-1 promotes mammary carcinogenesis. Cell 98: 137-146, 1999.

17. Whitham, S. E.; Murphy, G.; Angel, P.; Rahmsdorf, H. J.; Smith, B. J.; Lyons, A.; Harris, T. J.; Reynolds, J. J.; Herrlich, P.; Docherty, A. J.: Comparison of human stromelysin and collagenase by cloning and sequence analysis. Biochem. J. 240: 913-916, 1986.

18. Wilhelm, S. M.; Collier, I. E.; Kronberger, A.; Eisen, A. Z.; Marmer, B. L.; Grant, G. A.; Bauer, E. A.; Goldberg, G. I.: Human skin fibroblast stromelysin: structure, glycosylation, substrate specificity, and differential expression in normal and tumorigenic cells. Proc. Nat. Acad. Sci. 84: 6725-6729, 1987.

19. Ye, S.; Eriksson, P.; Hamsten, A.; Kurkinen, M.; Humphries, S. E.; Henney, A. M.: Progression of coronary atherosclerosis is associated with a common genetic variant of the human stromelysin-1 promoter which results in reduced gene expression. J. Biol. Chem. 271: 13055-13060, 1996.

References for MMP9 Section

1. Collier, I. E.; Bruns, G. A. P.; Goldberg, G. I.; Gerhard, D. S.: On the structure and chromosome location of the 72- and 92-kDa human type IV collagenase genes. Genomics 9: 429-434, 1991.

2. Huhtala, P.; Tuuttila, A.; Chow, L. T.; Lohi, J.; Keski-Oja, J.; Tryggvason, K.: Complete structure of the human gene for 92-kDa type IV collagenase: divergent regulation of expression for the 92- and 72-kilodalton enzyme genes in HT-1080 cells. J. Biol. Chem. 266: 16485-16490, 1991.

3. Linn, R.; DuPont, B. R.; Knight, C. B.; Plaetke, R.; Leach, R. I.: Reassignment of the 92-kDa type IV collagenase gene (CLG4B) to human chromosome 20. Cytogent. Cell Genet. 72: 159-161, 1996.

4. Nagase, H.; Barrett, A. J.; Woessner, J. F., Jr.: Nomenclature and glossary of the matrix metalloproteinases. Matrix Suppl. 1: 421-424, 1992.

5. St Jean, P. L.; Zhang, X. C.; Halt, B. K.; Lamlum, H.; Webster, M. W.; Steed, D. L.; Henney, A. M.; Ferrell, R. E.: Characterization of a dinucleotide repeat in the 92 kDa type IV collagenase gene (CLG4B), localization of CLG4B to chromosome 20 and the role of CLG4B in aortic aneurysmal disease. Ann. Hum. Genet. 59: 17-24, 1995.

6. Vu, T. H.; Shipley, J. M.; Bergers, G.; Berger, J. E.; Helms, J. A.; Hanahan, D.; Shapiro, S. D.; Senior, R. M.; Werb, Z.: MMP-9/gelatinase B is a key regulator of growth plate angiogenesis and apoptosis of hypertrophic chondrocytes. Cell 93: 411-422, 1998.

References for MMP13 Section

1. Freije, J. M. P.; Diez-Itza, I.; Balbin, M.; Sanchez, L. M.; Blasco, R.; Tolivia, J.; Lopez-Otin, C.: Molecular cloning and expression of collagenase-3, a novel human matrix metalloproteinase produced by breast carcinomas. J. Biol. Chem 269: 16766-16773, 1994.

2. Mitchell, P. G.; Magna, H. A.; Reeves, L. M.; Lopresti-Morrow, L. L.; Yocum, S. A.; Rosner, P. J.; Geoghegan, K. F.; Hambor, I. E.: Cloning, expression, and type II collagenolytic activity of matrix metalloproteinase-13 from human osteoarthritic cartilage. J. Clin. Invest. 97: 761-768, 1996.

3. Pendas, A. M.; Balbin, M.; Llano, E.; Jimenez, M. G.; Lopez-Otin, C.: Structural analysis and promoter characterization of the human collagenase-3 gene (MMP13). Genomics 40: 222-233, 1997.

4. Pendas, A. M.; Matilla, T.; Estivill, X.; Lopez-Otin, C.: The human collagenase-3 (CLG3) gene is located on chromosome 11q22.3 clustered to other members of the matrix metalloproteinase gene family. Genomics 26: 615-618, 1995.

5. Pendas, A. M.; Santamaria, I.; Alvarez, M. V.; Pritchard, M.; Lopez-Otin, C.: Fine physical mapping of the human matrix metalloproteinase genes clustered on chromosome 11q22.3. Genomics 37: 266-269, 1996.

6. Reboul, P.; Pelletier, J.-P.; Tardif, G.; Cloutier, J.-M.; Martel-Pelletier, J. The new collagenase, collagenase-3, is expressed and synthesized by human chondrocytes but not by synoviocytes: a role in osteoarthritis. J. Clin. Invest. 97: 2011-2019, 1996.

References for MMP14 Section

1. Holmbeck, K.; Bianco, P.; Caterina, J.; Yamada, S.; Kromer, M.; Kuznetsov, S. A.; Mankani, M.; Robey, P. G.; Poole, A. R.; Pidoux, I.; Ward, J. M.; Birkedal-Hansen, H.: MT1-MMP-deficient mice develop dwarfism, osteopenia, arthritis, and connective tissue disease due to inadequate collagen turnover. Cell 99: 81-92, 1999.

2. Mignon, C.; Okada, A.; Mattei, M. G.; Basset, P.: Assignment of the human membrane-type matrix metalloproteinase (MMP14) gene to 14q 11-q12 by in situ hybridization. Genomics 28: 360-361, 1995.

3. Sato, H.; Takino, T.; Okada, Y.; Cao, J.; Shinagawa, A.; Yamamoto, E.; Seiki, M.: A matrix metalloproteinase expressed on the surface of invasive tumor cells. Nature 370: 61-65, 1994.

4. Takino, T.; Sato, H.; Yamamoto, E.; Seiki, M.: Cloning of a human gene potentially encoding a novel matrix metalloproteinase having a C-terminal transmembrane domain. Gene 155: 293-298, 1995.

Sequences

A series of sequences are presented after the Abstract presented below. For the avoidance of doubt, these sequences are part of the description.

1. A pharmaceutical comprising a composition which comprises:
(a) a growth factor; and
(b) an inhibitor agent; and optionally
(c) a pharmaceutically acceptable carrier, diluent or excipient;
wherein the inhibitor agent can inhibit the action of at least one specific adverse protein (e.g. a specific protease) that is upregulated in a damaged tissue, such as a wound environment.

2. A pharmaceutical according to claim 1 wherein said growth factor is selected from one or more of Chrysalin, VEGF, EGF, PDGF, FGF, CTGF, KGF, TGF, CSF, or active variants, homologues, derivatives or fragments thereof.

3. A pharmaceutical according to claim 2 wherein said growth factor is selected from one or more of Chrysalin, VEGF, EGF, PDGF, FGF, CTGF-like, KGF-2, TGF-β, GM-CSF, or active variants, homologues, derivatives or fragments thereof.

4. A pharmaceutical according to any one of claims 1 to 3 wherein said growth factor is at least PDGF, or an active variant, homologue, derivative or fragment thereof.

5. A pharmaceutical according to any one of claims 1 to 4 wherein said inhibitor agent is an I:uPA and/or an I:MMP.

6. A pharmaceutical according to any one of claims 1 to 5 wherein said damaged tissue is a wound, preferably a chronic wound.

7. A pharmaceutical according to any one of claims 1 to 6 wherein said damaged tissue is a dermal ulcer.

8. A composition as defiled in any one of claims 1 to 7 for use in medicine.

9. Use of a composition as defined in any one of claims 1 to 7 in the manufacture of a pharmaceutical to treat chronic damaged tissue, such as chronic damaged wounds.

10. Use of a composition as defined in any one of claims 1 to 7 in the manufacture of a pharmaceutical to treat chronic dermal ulcers.

11. A method of therapy, said method comprising administering to a subject a composition as defined in any one of claims 1 to 7 and in an amount to treat damaged tissue, such as a wound.

12. A method according to claim 11 wherein said damaged tissue is a wound, preferably a chronic wound.

13. A method according to claim 11 or 12 wherein said damaged tissue is a dermal ulcer.

14. A process for preparing a composition as defined in any one of claims 1 to 7; said process comprising the steps of:
(i) performing an assay to identify one or more agents that are capable of acting as an inhibitor agent as defined in any one of claims 1 to 7;
(ii) admixing one or more of said agent(s) with a growth factor and optionally a pharmaceutically acceptable carrier, diluent or excipient.

15. A process according to claim 14 wherein said process also includes the subsequent step of:
(iii) administering said composition to a subject in need of same.

16. A process for preparing a pharmaceutical for use in treating damaged tissue, such as a wound; the process comprising forming a composition by admixing (a) a growth factor with (b) an inhibitor agent; and (c) optionally also admixing with a pharmaceutically acceptable carrier, diluent or excipient; wherein the inhibitor agent can inhibit the action of at least one specific adverse protein (e.g. a specific protease) that is upregulated in a damaged tissue, such as a wound, environment.

17. A pack comprising at least two compartments; wherein first of said compartments houses a growth factor; and wherein second of said compartments houses an inhibitor agent, wherein the inhibitor agent can inhibit the action of at least one specific adverse protein (e.g. a specific protease) that is upregulated in a damaged tissue, such as a wound, environment.

18. Use of a growth factor as defined in any one of claims 1 to 7 in the manufacture of a pharmaceutical to treat a subject that is being treated with an inhibitor agent as defined in any one of claims 1 to 7.

19. Use of an inhibitor agent as defined in any one of claims 1 to 7 in the manufacture of a pharmaceutical to treat a subject that is being treated with a growth factor as defined in any one of claims 1 to 7.

20. A method of therapy, said method comprising administering to a subject a composition as defined in any one of claims 1 to 7 and in an amount to treat (e.g. heal) damaged tissue, such as a wound; wherein all or some (preferably all) of said growth factor as defined in any one of claims 1 to 7 is administered topically and wherein all or some (preferably all) of said inhibitor agent as defined in any one of claims 1 to 7 as administered topically.

21. Use of a composition as defined in any one of claims 1 to 7 in the manufacture of a pharmaceutical to treat chronic damaged tissue, such as chronic damaged wounds; wherein all or some (preferably all) of said growth factor as defined in any one of claims 1 to 7 is administered topically and wherein all or some (preferably all) of said inhibitor agent as defined in any one of claims 1 to 7 as administered topically.

22. Use of a growth factor as defined in any one of claims 1 to 7 in the manufacture of a pharmaceutical to treat a subject that is being treated with an inhibitor agent as defined in any one of claims 1 to 7; wherein all or some (preferably all) of said growth factor as defined in any one of claims 1 to 7 is administered topically and wherein all or some (preferably all) of said inhibitor agent as defined in any one of claims 1 to 7 as administered topically.

23. A pharmaceutical comprising:
(a) a growth factor;
(b) an i:UPA and/or an iMMP; and optionally
(c) a pharmaceutically acceptable carrier, diluent or excipient;
wherein the iUPA and/or the iMMP can inhibit the action of at least one specific adverse protein (e.g. a specific protease) that is upregulated in a damaged tissue, such as a wound, environment.

24. Use of a pharmaceutical composition according to claim 8 to treat damaged tissue, such as wound.

25. A pharmaceutical composition comprising:
(i) an i:UPA
(ii) an iMMP; and optionally
(iii) a pharmaceutically acceptable carrier, diluent or excipient;
wherein the iUPA and/or the iMMP can inhibit the action of at least one specific adverse protein (e.g. a specific protease) that is upregulated in a damaged tissue, such as a wound, environment.

26. A pharmaceutical composition according to claim 25 wherein the composition also comprises a growth factor.

27. A pharmaceutical composition according to claim 26 wherein said growth factor is an exogeneous growth factor.

28. The invention according to any one of the preceding claims wherein the inhibitor is at least an i:UPA.

29. The invention according to any one of the preceding claims wherein the inhibitor is at least an i:MMP; wherein said MMP is MMP 3 and/or MMP 13.
